SEQUENCE LISTING (4 sequences)

SEQ ID NO: 1 - DNA, 34 bp, Artificial
  primer
ggggctcgaggcagggatggagaccaaaggctac

SEQ ID NO: 2 - DNA, 30 bp, Artificial
  primer
ggatacccatcacttcttctagacgacagg

SEQ ID NO: 3 - DNA, 30 bp, Artificial
  primer
agtgggtattaaacaagaaccagatgacgg

SEQ ID NO: 4 - DNA, 34 bp, Artificial
  primer
gggaggtaccttctgggcagcgggcagtcacttc

(19) United States
(12) Patent Application Publication
(10) Pub. No.: US 2009/0253687 A1
(43) Pub. Date: Oct. 8, 2009

(54) Fused Heterocyclic Compounds and Their Use as Mineralocorticoid Receptor Ligands

(76) Inventors: Shoji Fukumoto, Osaka (JP); Nobuyuki Matsunaga, Osaka (JP); Taiichi Ohra, Osaka (JP); Norio Ohyabu, Osaka (JP); Tomoaki Hasui, Osaka (JP); Takashi Motoyaji, Osaka (JP); Christopher Stephen Siedem, Boulder, CO (US); Tony Pisal Tang, Boulder, CO (US); Lisa A. Demeese, Boulder, CO (US); Cassandra Gauthier, Grand Junction, CO (US)

Correspondence Address: WENDEROTH, LIND & PONACK, L.L.P., 1030 15th Street, N.W.,, Suite 400 East, Washington, DC, 20005-1503, US

(21) Appl. No.: 12/087,148

(22) PCT Filed: Dec. 27, 2006
(86) PCT No.: PCT/JP2006/326367

§ 371 Date: Dec. 17, 2008

Related U.S. Application Data
(60) Provisional application No. 60/754,416, filed on Dec. 28, 2005.
(60) Provisional application No. 60/818,803, filed on Jul. 6, 2006.

(51) Int. Cl. A61K 31/542; C07D 285/16; C07D 265/36; A61K 31/538; C07D 279/16; A61K 31/5415; C07D 513/02; A61P 9/00

(52) U.S. Cl. 514/222.8; 544/10; 544/105; 514/230.5; 544/51; 514/224.2; 544/73; 544/48

(57) ABSTRACT

The present invention relates to wherein each symbol is as defined in the specification. The compound has a superior mineral corticoidreceptorantagonistic action and is useful as an agent for the prophylaxis or treatment of hypertension, cardiac failure and the like, a compound having a fused heterocycle, or a prodrug thereof, or a salt thereof; and an agent for the prophylaxis or treatment of hypertension, cardiac failure and the like.

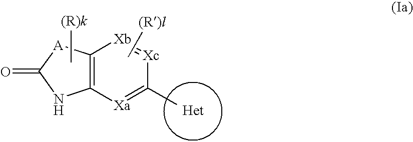

This application is based on application Nos. 60/754,416 and 60/818,803 filed in USA, the contents of which are incorporated hereinto by reference.

### TECHNICAL FIELD

The present invention relates to a compound having a fused heterocycle, which is useful as an agent for the prophylaxis or treatment of hypertension, cardiac failure and the like, a prodrug thereof or a salt thereof; an agent containing same, which is used for the prophylaxis or treatment of hypertension, cardiac failure and the like; and the like.

### BACKGROUND ART

Aldosterone is a final product of renin-angiotensin-aldosterone system (RAAS), which binds to a mineralocorticoid receptor (MR; aldosterone receptor). Since it expresses actions to adjust water and electrolyte, microvessel contraction, ischemia, induction of inflammation of blood vessel, promotion of tissue fibrosis and the like, it is suggested that excess production or secretion of aldosterone is involved in the diseases such as hypertension, congestive heart failure, arteriosclerosis, cerebral infarction, acute coronary diseases, nephropathy and the like. It has been reported that hypertension is developed in primary aldosteronism with increased secretion of aldosterone from the adrenal gland, and the complications in the cardiac or blood vessel system and kidney are observed at high frequency (see Journal of Clinical Endocrinology and Metabolism, 2003, vol. 88, p. 2364-2372). In addition, spironolactone and eplerenone having a steroid structure, which are used clinically, show a hypotensive action in patients with hypertension. In a large-scale clinical test, RALES (Randomized Aldactone Evaluation Study), it has been reported that spironolactone decreases the death rate of patients with severe cardiac failure (see New England Journal of Medicine, 1999, vol. 341, p. 709-717) and, in EPHESUS (Eplerenone Post-AMI Heart Failure Efficacy and Survival Study), it has been reported that eplerenone decreases the death rate and cardiovascular incidents in patients with cardiac infarction suffering from the complication of the decreased left ventricle function and cardiac failure (see New England Journal of Medicine, 2003, vol. 48, p. 1309-1321), and the usefulness of mineralocorticoid receptor antagonists in the treatment of hypertension and cardiac failure is being established.

As the mineralocorticoid receptor antagonist, compounds having a steroid structure such as canrenone and the like have been reported besides the above-mentioned spironolactone and eplerenone, and, as compounds having a non-steroidal skeleton, naphthalene derivative (see Biochemical Pharmacology, 1974, vol. 23, p. 1493), benzodiazepine derivative (see U.S. Pat. No. 4,251,443), indole derivative (see U.S. Pat. No. 4,179,503) and the like have been reported.

In addition, compounds having a non-steroidal skeleton, which interact with steroid hormone receptors including a mineralocorticoid receptor as a site of action, are disclosed in U.S. Pat. No. 6,964,973, WO03/078394, WO04/052847, WO05/066153, WO05/066161, WO05/087740, WO05/092854, WO05/097118, J. Comb. Chem., vol. 7, page 567-573 (2005) and the like. However, a compound having a structure as in the present invention is not disclosed.

Compounds having a fused heterocycle which does not interact with a steroid hormone receptor as a site of action are disclosed, for example, in WO01/062756, WO03/042207, WO03/042211, WO03/097639, WO04/050659, WO04/072033, WO04/111036 and the like as a series of compounds having an ALK5 receptor antagonistic action. In addition, compounds having a hypotensive action, an anti-inflammatory action, and the like are disclosed in DE-A-2837161, EP-A-122494, EP-A-132817, DE-A-3536030, WO87/03201, EP-A-272914, U.S. Pat. No. 4,721,784, EP-A-326307, EP-A-509845, Heterocyclic Communications, vol. 9, p. 51-56 (2003), WO98/07720, WO05/007652, Chimia, vol. 51 (11), p. 715-719 (2003), EP-A-385850, WO96/01254 and the like.

### DISCLOSURE OF THE INVENTION

As a result of the intensive studies of the compounds having a mineralocorticoid receptor antagonistic action, the present inventors have surprisingly found compounds represented by the following formulas (Ia′) and (I′) (particularly, compounds represented by the formulas (Ia) and (I)), a salt thereof or a prodrug thereof has a superior mineralocorticoid receptor antagonistic action, which resulted in the completion of the present invention.

Accordingly, the present invention provides the following.
[1] A compound of the formula (Ia):

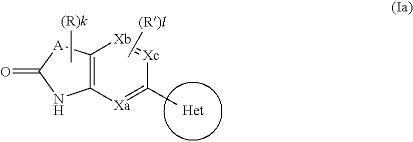

wherein
A is a group represented by the formula:

—X₁ [symbol] X₂ [symbol] X₃—
wherein
X₁ and X₂ are the same or different and each is a chemical bond, CH₂, CH, O, NH, N, S, SO or SO₂;
X₃ is CH₂, CH, O, NH, N, S, SO or SO₂; and
[symbol] is a single bond or a double bond;
provided that
when
—X₁ [symbol] X₂— is —X₁═X₂—,
then
—X₂ [symbol] X₃— should be —X₂—X₃—;
   R and R′ are the same or different and each is an optionally substituted aliphatic hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group or an acyl group, or two R optionally form a spiro ring together with a carbon atom they are bonded to;
   k is an integer of 0 to 4;
   l is an integer of 0 to 3;

### Xₐ is CH or N;

### X_{b} is CH or N;

### X_{c} is CH or N; and

a group represented by the formula:

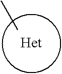

is a heterocyclic group represented by the formula:

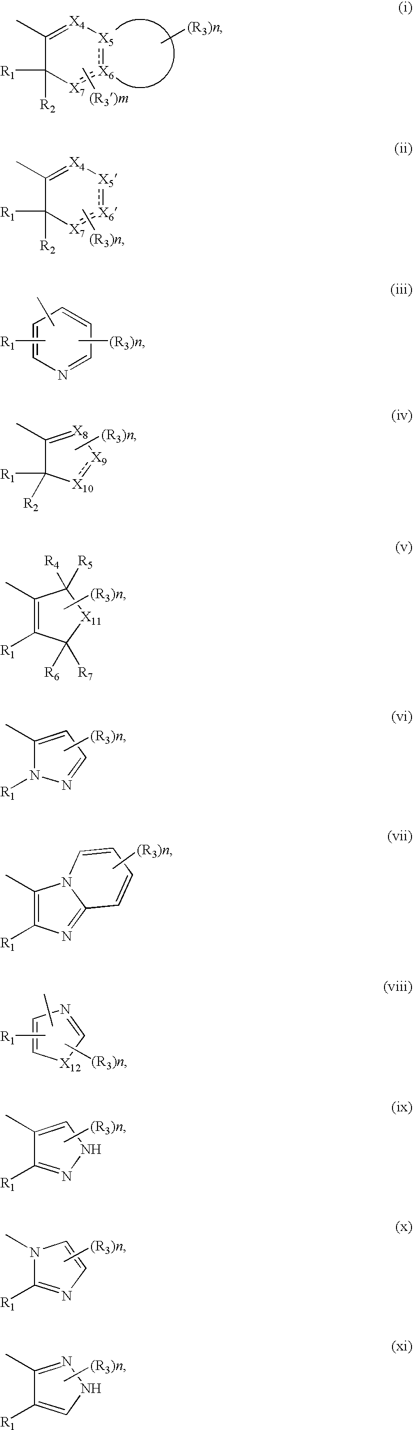

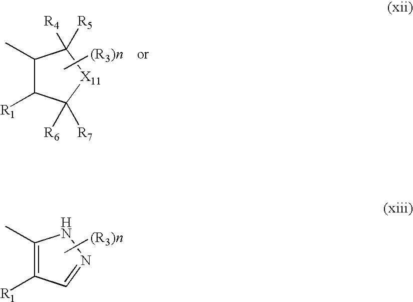

wherein
the formula:

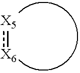

which partially constitutes the fused ring in the heterocyclic group represented by the formula (i), is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group, or
two R₃ optionally form, together with two adjacent atoms they are bonded to, a 3- to 7-membered ring which optionally contains, as a ring-constituting member, one or more members selected from O, N, S, SO and SO₂;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₆ and R₇ in combination optionally form an oxo group;
provided that at least one of a pair of R₄ and R₅ and a pair of R₆ and R₇ should form an oxo group;
m and n are the same or different and each is an integer of 0 to 4;
X₄ is CH or N;
X₅ and X₆ are the same or different and each is CH, C or N;
X₅′ and X₆′ are the same or different and each is CH₂, CH, NH, N, O, S, SO or SO₂;
X₇ is CH₂, CH, NH, N, O, S, SO or SO₂;
X₈ is CH or N;
X₉ is CH₂, CH, NH, N, O, S, SO or SO₂;
X₁₀ is CH₂, CH, NH, N, O, S, SO or SO₂;
X₁₁ is NH, O, S, SO or SO₂;
X₁₂ is O or S; and
[symbol] is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇;
   with the proviso that
   1) when the group represented by the formula:

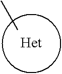

is a heterocyclic group represented by the formula:

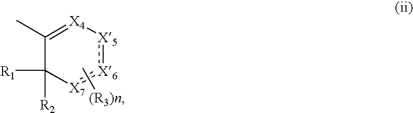

then at least one of R₁ and R₂ should be an optionally substituted aryl group or an optionally substituted heteroaryl group,
2) when the group represented by the formula:

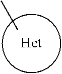

is a heterocyclic group represented by the formula:

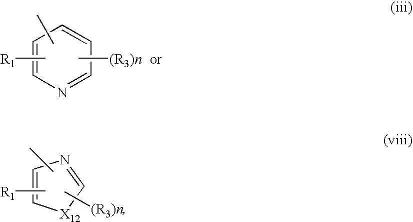

then the carbon atom to which the group represented by the formula:

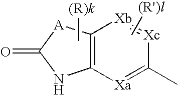

is bonded and the carbon atom to which R₁ is bonded should be adjacent to each other, and R₁ should be an optionally substituted aryl group or an optionally substituted heteroaryl group,
3) when the group represented by the formula:
—X₁ [symbol] X₂ [symbol] X₃— is —CH₂—O—, and the group represented by the formula:

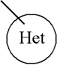

is a heterocyclic group represented by the formula:

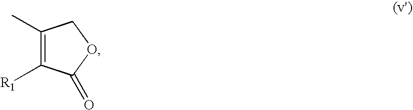

then R₁ should not be phenyl, 4-methoxyphenyl, 3,4-dimethoxyphenyl and 4-chlorophenyl,
4) when the group represented by the formula:
—X₁ [symbol] X₂ [symbol] X₃— is —CH₂—O—, and the group represented by the formula:

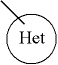

is a heterocyclic group represented by the formula:

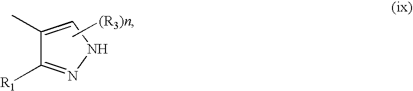

then R₁ should not be an optionally substituted 2-pyridyl,
5) when the group represented by the formula:
—X₁ [symbol] X₂ [symbol] X₃— is —CH₂—O—, and the group represented by the formula:

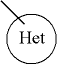

is a heterocyclic group represented by the formula:

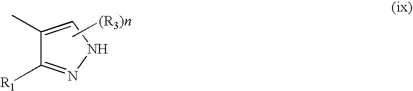

wherein R₁ is an optionally substituted phenyl, then —NH— group in the pyrazole ring as illustrated above should be substituted by R₃,

6) when the group represented by the formula:
—X₁ [symbol] X₂ [symbol] X₃— is —O—, —CH₂—O—, —CH₂—S— or —CH═CH—, and the group represented by the formula:

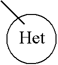

is a heterocyclic group represented by the formula:

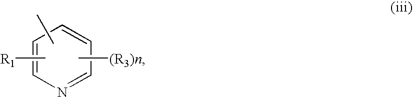

then R₁ should not be a halogen atom and trifluoromethyl,
7) when the group represented by the formula:
—X₁ [symbol] X₂ [symbol] X₃— is —NH— or —CH₂—NH—, and the group represented by the formula:

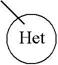

is a heterocyclic group represented by the formula:

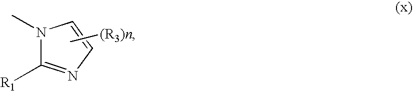

then R₁ should not be an alkyl group,
8) when the group represented by the formula:
—X₁ [symbol] X₂ [symbol] X₃— is —CH₂—O—, and the group represented by the formula:

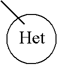

is a heterocyclic group represented by the formula:

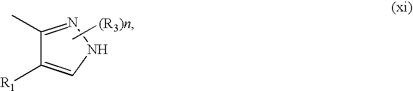

then R₁ should be an optionally substituted aryl group or an optionally substituted heteroaryl group,
9) when the group represented by the formula:
—X₁ [symbol] X₂ [symbol] X₃— is —S— or —CH₂—O—, and the group represented by the formula:

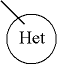

is a heterocyclic group represented by the formula:

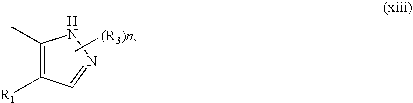

then R₁ should not be a halogen atom, and
10) when the group represented by the formula:

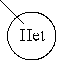

is a heterocyclic group represented by the formula:

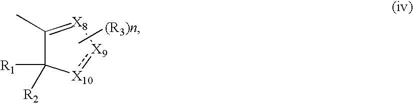

then at least one of R₁ and R₂ should be an optionally substituted aryl group or an optionally substituted heteroaryl group,
or a salt thereof [hereinafter sometimes to be abbreviated as compound (Ia)].
[2] A compound of the formula (I):

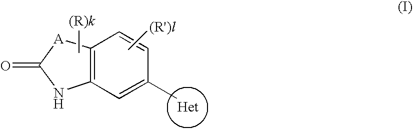

wherein
A is a group represented by the formula:

—X₁ [symbol] X₂ [symbol] X₃—
wherein
X₁ and X₂ are the same or different and each is a chemical bond, CH₂, CH, O, NH, N, S, SO or SO₂;
X₃ is CH₂, CH, O, NH, N, S, SO or SO₂; and
[symbol] is a single bond or a double bond;
provided that
when
—X₁ [symbol] X₂— is —X₁═X₂—,
then
—X₂ [symbol] X₃— should be —X₂—X₃—;
   R and R′ are the same or different and each is an optionally substituted aliphatic hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group or an acyl group, or two R optionally form a spiro ring together with a carbon atom they are bonded to;
   k is an integer of 0 to 4;
   l is an integer of 0 to 3; and
   a group represented by the formula:

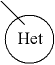

is a heterocyclic group represented by the formula:

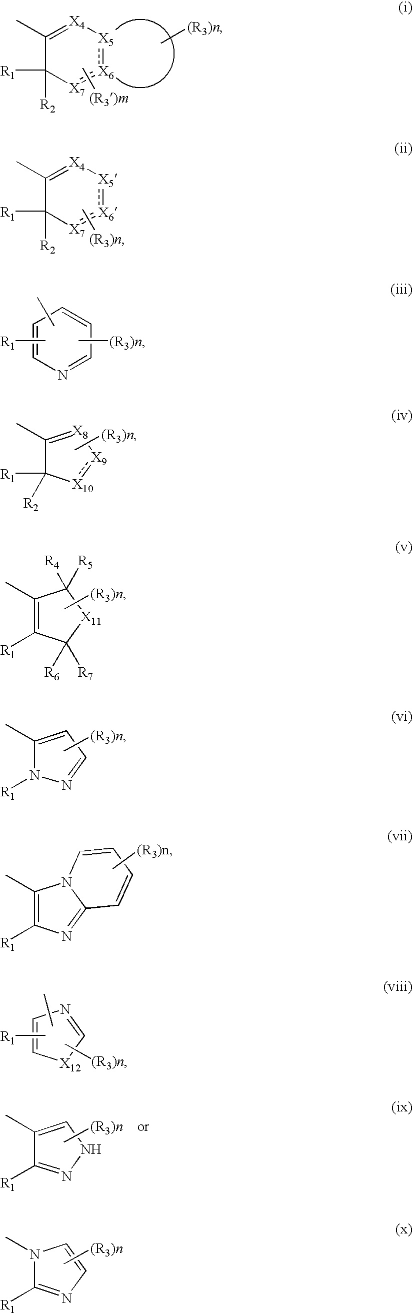

wherein
the formula:

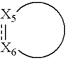

which partially constitutes the fused ring in the heterocyclic group represented by the formula (i), is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group, or
two R₃ optionally form, together with two adjacent atoms they are bonded to, a 3- to 7-membered ring which optionally contains, as a ring-constituting member, one or more members selected from O, N, S, SO and SO₂;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₆ and R₇ in combination optionally form an oxo group;
provided that at least one of a pair of R₄ and R₅ and a pair of R₆ and R₇ should form an oxo group;
m and n are the same or different and each is an integer of 0 to 4;
X₄ is CH or N;
X₅ and X₆ are the same or different and each is CH, C or N;
X₅′ and X₆′ are the same or different and each is CH₂, CH, NH, N, O, S, SO or SO₂;
X₇ is CH₂, CH, NH, N, O, S, SO or SO₂;
X₈ is CH or N;
X₉ is CH₂, CH, NH, N, O, S, SO or SO₂;
X₁₀ is CH₂, CH, NH, N, O, S, SO or SO₂;
X₁₁ is NH, O, S, SO or SO₂;
X₁₂ is O or S; and
[symbol] is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇;
   with the proviso that
   1) when the group represented by the formula:

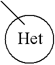

is a heterocyclic group represented by the formula:

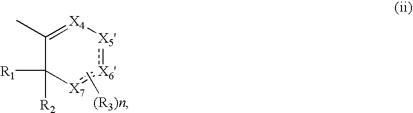

then at least one of R₁ and R₂ should be an optionally substituted aryl group or an optionally substituted, heteroaryl group,
2) when the group represented by the formula:

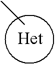

is a heterocyclic group represented by the formula:

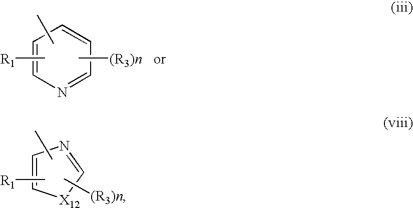

then the carbon atom to which the group represented by the formula:

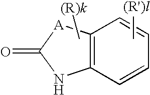

is bonded and the carbon atom to which R₁ is bonded should be adjacent to each other, and R₁ should be an optionally substituted aryl group or an optionally substituted heteroaryl group,
3) when the group represented by the formula:
—X₁ [symbol] X₂ [symbol] X₃— is —CH₂—O—, and the group represented by the formula:

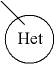

is a heterocyclic group represented by the formula:

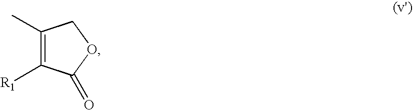

then R₁ should not be phenyl, 4-methoxyphenyl, 3,4-dimethoxyphenyl and 4-chlorophenyl,
4) when the group represented by the formula:
—X₁ [symbol] X₂ [symbol] X₃— is —CH₂—O—, and the group represented by the formula:

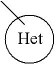

is a heterocyclic group represented by the formula:

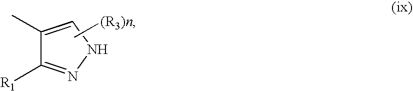

then R₁ should not be an optionally substituted 2-pyridyl,
5) when the group represented by the formula:
—X₁ [symbol] X₂ [symbol] X₃— is —CH₂—O—, and the group represented by the formula:

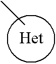

is a heterocyclic group represented by the formula:

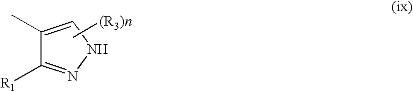

wherein R₁ is an optionally substituted phenyl, then —NH— group in the pyrazole ring as illustrated above should be substituted by R₃,

6) when the group represented by the formula:
—X₁ [symbol] X₂ [symbol] X₃— is —O—, —CH₂—O—, —CH₂—S— or —CH═CH—, and the group represented by the formula:

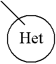

is a heterocyclic group represented by the formula:

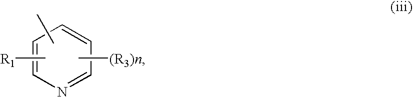

then R₁ should not be a halogen atom and trifluoromethyl,
7) when the group represented by the formula:
—X₁ [symbol] X₂ [symbol] X₃— is —NH— or —CH₂—NH—, and the group represented by the formula:

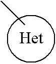

is a heterocyclic group represented by the formula:

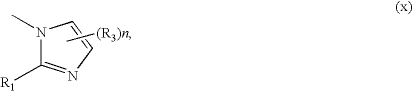

then R₁ should not be an alkyl group, and
8) when the group represented by the formula:

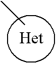

is a heterocyclic group represented by the formula:

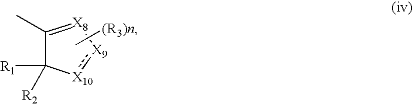

then at least one of R₁ and R₂ should be an optionally substituted aryl group or an optionally substituted heteroaryl group,
or a salt thereof [hereinafter sometimes to be abbreviated as compound (I)].
[3] The compound of the aforementioned [1], wherein none or one of X₁, X₂ and X₃ is a hetero atom, or a salt thereof.
[4] The compound of the aforementioned [1], wherein
   A is a group represented by the formula:

—X₁ [symbol] X₂ [symbol] X₃—
wherein
X₁ is a chemical bond or CH₂;
X₂ is a chemical bond, CH₂, CH, O, NH, N, S, SO or SO₂; and
X₃ is CH₂, CH, O, NH, N, S, SO or SO₂;
   or a salt thereof.
[5] The compound of the aforementioned [1], excluding a compound wherein consecutive three or more of X₄, X₅, X₆ and X₇ or consecutive three or more of X₄, X₅′, X₆′ and X₇ are hetero atoms, or a salt thereof.
[6] The compound of the aforementioned [1], wherein the group represented by the formula:

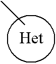

is a heterocyclic group represented by the formula:

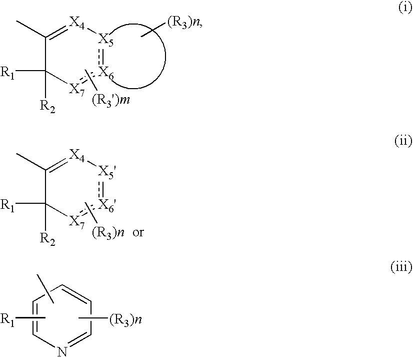

wherein R₁, R₂, R₃, R₃′, m, n, X₄, X₅, X₅′, X₆, X₆′ and X₇ are each as defined in the aforementioned [1],
   or a salt thereof.
[7] The compound of the aforementioned [1], wherein the group represented by the formula:

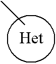

is a heterocyclic group represented by the formula:

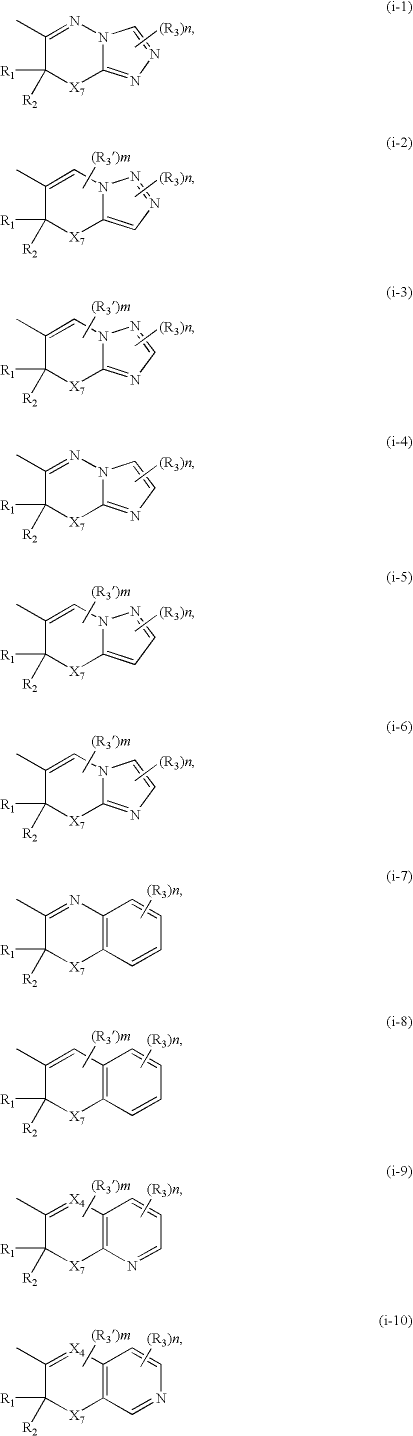

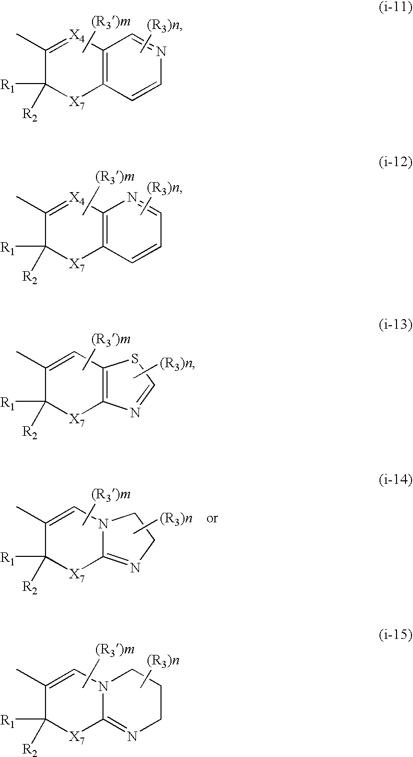

wherein
R₁, R₂, n and X₄ are each as defined in the aforementioned [1];
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;
X₇ is O, S, SO or SO₂; and
m is an integer of 0 to 1,
   or a salt thereof.
[8] The compound of the aforementioned [1], wherein the group represented by the formula:

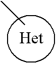

is a heterocyclic group represented by the formula:

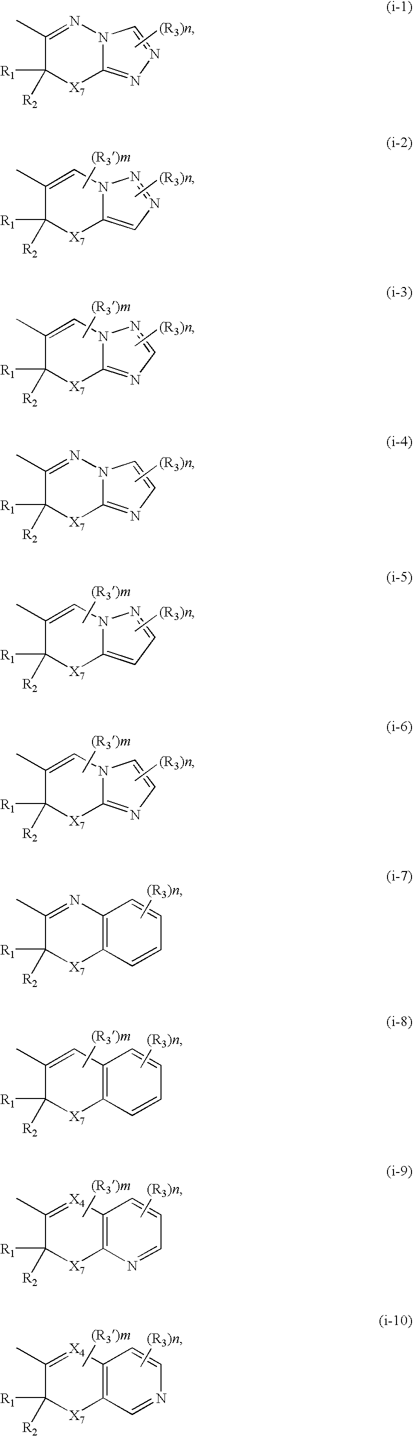

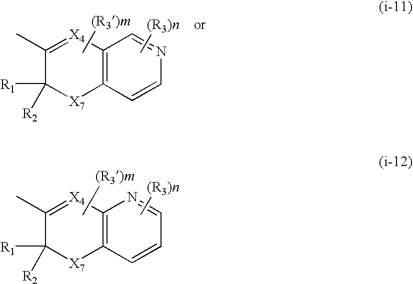

wherein
R₁, R₂, n and X₄ are each as defined in the aforementioned [1];
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;
X₇ is O, S, SO or SO₂; and
m is an integer of 0 to 1,
   or a salt thereof.
[9] The compound of the aforementioned [1], wherein the group represented by the formula:

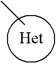

is a heterocyclic group represented by the formula:

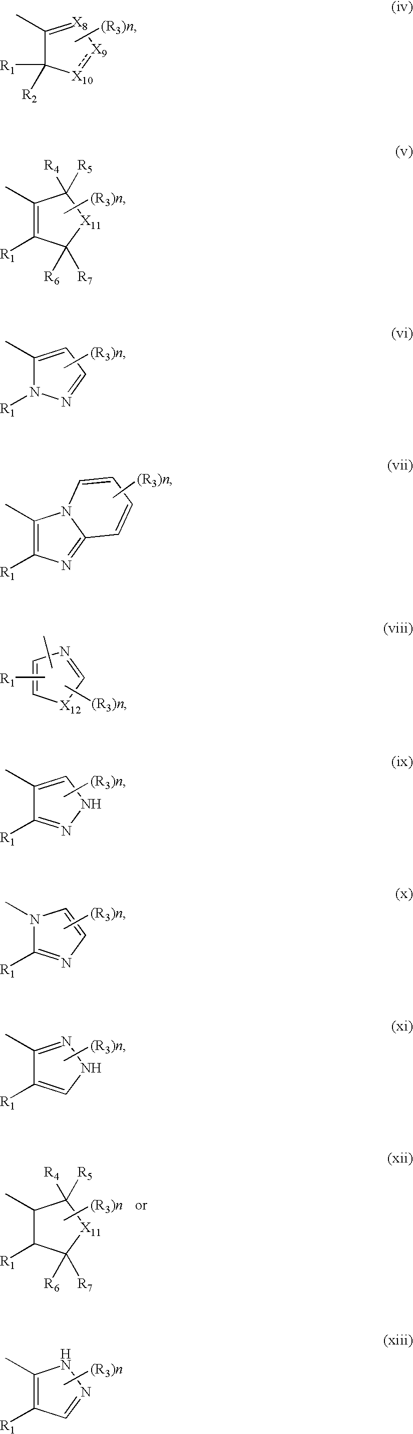

wherein R₁, R₂, R₃, R₄, R₅, R₆, R₇, n, X₈, X₉, X₁₀, X₁₁ and X₁₂ are each as defined in the aforementioned [1],
   or a salt thereof.
[10] The compound of the aforementioned [1], wherein the group represented by the formula:

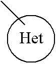

is a heterocyclic group represented by the formula:

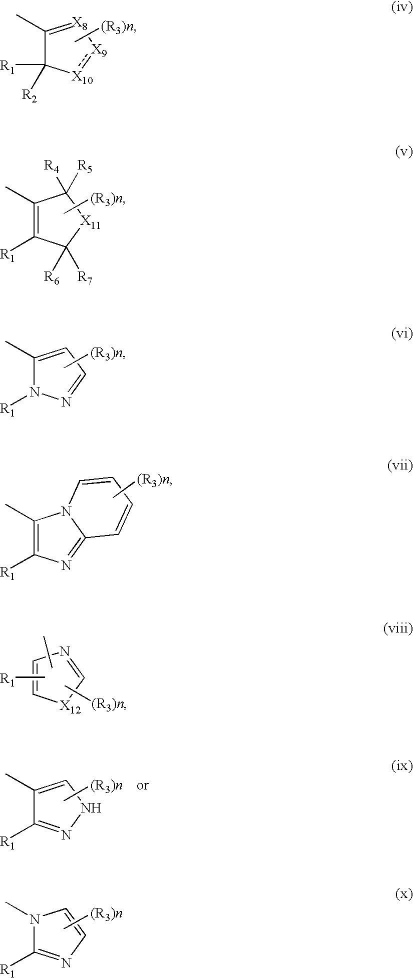

wherein R₁, R₂, R₃, R₄, R₅, R₆, R₇, n, X₈, X₉, X₁₀, X₁₁ and X₁₂ are each as defined in the aforementioned [1],
   or a salt thereof.
[11] The compound of the aforementioned [1], wherein the group represented by the formula:

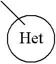

is a heterocyclic group represented by the formula:

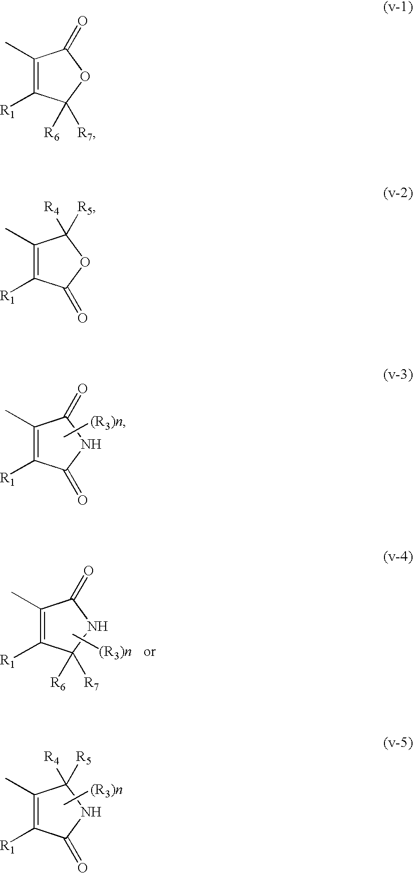

wherein
R₁, R₄, R₅, R₆ and R₇ are as defined in the aforementioned [1];
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group; and
n is an integer of 0 to 1,
   or a salt thereof.
[12] The compound of the aforementioned [1], wherein the group represented by the formula:

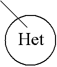

is a heterocyclic group represented by the formula:

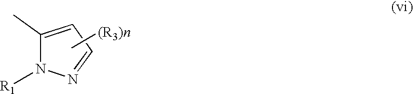

wherein
R₁ is as defined in the aforementioned [1];
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group; and
n is an integer of 0 to 2,
   or a salt thereof.
[13] The compound of the aforementioned [1], wherein the group represented by the formula:

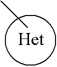

is a heterocyclic group represented by the formula:

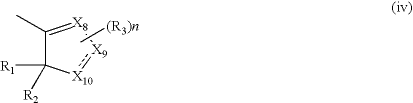

wherein
R₁, R₂, n, X₈, X₉ and X₁₀ are each as defined in the aforementioned [1]; and
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group,
   or a salt thereof.
[14] The compound of the aforementioned [1], wherein the group represented by the formula:

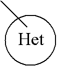

is a heterocyclic group represented by the formula:

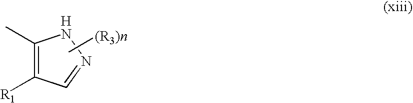

wherein
R₁ is as defined in the aforementioned [1];
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group; and
n is an integer of 0 to 2,
   or a salt thereof.
[15] The compound of the aforementioned [1], wherein the group represented by the formula:

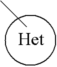

is a heterocyclic group represented by the formula:

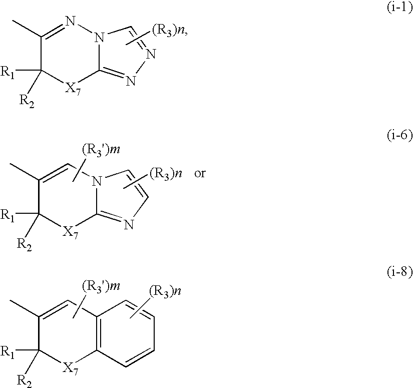

wherein
R₁, R₂ and n are each as defined in the aforementioned [1];
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group;
X₇ is O, S, SO or SO₂; and
m is an integer of 0 to 1,
   or a salt thereof.
[16] The compound of the aforementioned [1], wherein when one of R₁ and R₂ is a hydrogen atom, then the other should not be a hydrogen atom,
   or a salt thereof.
[17] 6-(7-phenyl-7H-[1,2,4]triazolo[3,4-b][1,3,4]thiadiazin-6-yl)-2H-1,4-benzoxazin-3(4H)-one,
6-[2-(4-fluorophenyl)-2H-thiochromen-3-yl]-2H-1,4-benzoxazin-3(4H)-one,
3-(3-oxo-3,4-dihydro-2H-1,4-benzoxazin-6-yl)-2-phenyl-2H-thiochromene-7-carbonitrile,
6-(2-amino-6-phenyl-6H-1,3-thiazin-5-yl)-2H-1,4-benzoxazin-3(4H)-one,
6-[7-(2-chlorophenyl)-7H-[1,2,4]triazolo[3,4-b][1,3,4]thiadiazin-6-yl]-2H-1,4-benzoxazin-3(4H)-one,
8-fluoro-6-[7-(4-fluorophenyl)-7H-[1,2,4]triazolo[3,4-b][1,3,4]thiadiazin-6-yl]-2H-1,4-benzoxazin-3(4H)-one,
6-[7-(4-fluorophenyl)-7H-[1,2,4]triazolo[3,4-b][1,3,4]thiadiazin-6-yl]-8-methyl-2H-1,4-benzoxazin-3(4H)-one,
8-chloro-6-[7-(4-fluorophenyl)-7H-imidazo[2,1-b][1,3]thiazin-6-yl]-2H-1,4-benzoxazin-3(4H)-one,
3-(4-fluorophenyl)-4-(3-oxo-3,4-dihydro-2H-benzo[b][1,4]oxazin-6-yl)-1-phenyl-1H-pyrrole-2,5-dione,
6-(1-o-tolyl-3-(trifluoromethyl)-1H-pyrazol-5-yl)-2H-benzo[b][1,4]oxazin-3(4H)-one,
6-(1-(4-fluoro-2-methylphenyl)-3-(trifluoromethyl)-1H-pyrazol-5-yl)-2H-benzo[b][1,4]oxazin-3(4H)-one,
8-fluoro-6-(1-(4-fluoro-2-methylphenyl)-3-(trifluoromethyl)-1H-pyrazol-5-yl)-2H-benzo[b][1,4]oxazin-3(4H)-one,
6-(1-(4-fluoro-2-methylphenyl)-3-(trifluoromethyl)-1H-pyrazol-5-yl)-8-methyl-2H-benzo[b][1,4]oxazin-3(4H)-one,
6-(1,3-dimethyl-4-phenyl-1H-pyrazol-5-yl)-2H-benzo[b][1,4]oxazin-3(4H)-one,
6-(1-(4-chloro-2-methylphenyl)-3-(trifluoromethyl)-1H-pyrazol-5-yl)-2H-benzo[b][1,4]oxazin-3(4H)-one,
6-(1-(2,5-dimethylphenyl)-3-(trifluoromethyl)-1H-pyrazol-5-yl)-2H-benzo[b][1,4]oxazin-3(4H)-one,
6-[1-(4-fluoro-2-methylphenyl)-3-(trifluoromethyl)-1H-pyrazol-5-yl]-8-methyl-2H-pyrido[3,2-b][1,4]oxazin-3(4H)-one, or
6-(3-(1,1-difluoroethyl)-1-(4-fluoro-2-methylphenyl)-1H-pyrazol-5-yl)-8-methyl-2H-benzo[b][1,4]oxazin-3(4H)-one,
   or a salt thereof.
[18] A prodrug of a compound of the aforementioned [1] or a salt thereof.
[19] A pharmaceutical composition comprising a compound of the aforementioned [1] or a pharmaceutically acceptable salt thereof or a prodrug thereof, in admixture with a pharmaceutically acceptable carrier.
[20] A method for inhibiting the mineralocorticoid receptor activity in a mammal, comprising administering an effective amount of a compound of the aforementioned [1] or a pharmaceutically acceptable salt thereof or a prodrug thereof to said mammal.
[21] A method for preventing or treating a disease or condition mediated by the mineralocorticoid receptor activation in a mammal, comprising administering an effective amount of a compound of the aforementioned [1] or a pharmaceutically acceptable salt thereof or a prodrug thereof to said mammal.
[22] A method for inhibiting the mineralocorticoid receptor activity in a mammal, comprising administering an effective amount of a compound of formula (Ia′):

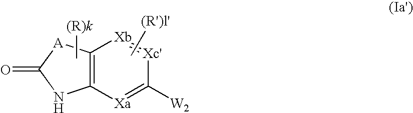

wherein

### X_{c}′ is C—W₁ or N;

W₁ and W₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
l′ is an integer of 0 to 2; and
A, R, R′ Xₐ, X_{b} and k are each as defined in the aforementioned [1];
with the proviso that
1) at least one of W₁ and W₂ should be an optionally substituted cyclic group,
2) when W₂ is a hydrogen atom, then W₁ should not be an optionally substituted phenyl, and
3) at least one of Xₐ, X_{b} and X_{c}′ should be N, a pharmaceutically acceptable salt thereof [hereinafter sometimes to be abbreviated as compound (Ia′)] or a prodrug thereof to said mammal.
[23] A method for inhibiting the mineralocorticoid receptor activity in a mammal, comprising administering an effective amount of a compound of formula (I′):

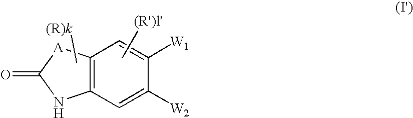

wherein
W₁ and W₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
l′ is an integer of 0 to 2; and
A, R, R′ and k are each as defined in the aforementioned [2];
with the proviso that
1) at least one of W₁ and W₂ should be an optionally substituted cyclic group, and
2) when W₂ is a hydrogen atom, then W₁ should not be an optionally substituted phenyl,
a pharmaceutically acceptable salt thereof [hereinafter sometimes to be abbreviated as compound (I′)] or a prodrug thereof to said mammal.

Each symbol in the formulas (Ia), (I), (Ia′) and (I′) is described in detail in the following.

In the present specification, the term “lower” means 1 to 6 carbon atoms, preferably 1 to 4 carbon atoms.

As the “halogen atom” for R, R′, R₁, R₂, R₃, R₃′, R₄, R₅, R₆, R₇, W₁ or W₂, for example, fluorine, chlorine, bromine and iodine can be mentioned.

As the “aliphatic hydrocarbon group” of the “optionally substituted aliphatic hydrocarbon group” for R or R′, an aliphatic chain hydrocarbon group and an alicyclic hydrocarbon group (non-aromatic cyclic hydrocarbon group) can be mentioned.

As the “aliphatic chain hydrocarbon group” exemplified for the “aliphatic hydrocarbon group”, for example, a linear or branched chain aliphatic hydrocarbon group such as an alkyl group, an alkenyl group, an alkynyl group and the like can be mentioned.

As used herein, the “alkyl group” may be linear or branched and, for example, a C₁₋₁₀ alkyl group (preferably a C₁₋₆ alkyl group etc.) such as methyl, ethyl, n-propyl, isopropyl, n-butyl, isobutyl, sec-butyl, tert-butyl, n-pentyl, isopentyl, neopentyl, 1-methylpropyl, n-hexyl, isohexyl, 1,1-dimethylbutyl, 2,2-dimethylbutyl, 3,3-dimethylbutyl, 3,3-dimethylpropyl, 2-ethylbutyl, n-heptyl, 1-methylheptyl, 1-ethylhexyl, n-octyl, 1-methylheptyl, nonyl and the like, and the like can be mentioned.

The “alkenyl group” may be linear or branched and, for example, a C₂₋₁₀ alkenyl group (preferably a C₂₋₆ alkenyl group etc.) such as vinyl, allyl, isopropenyl, 2-methylallyl, 1-propenyl, 2-methyl-1-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl, 5-hexenyl and the like, and the like can be mentioned.

The “alkynyl group” may be linear or branched and, for example, a C₂₋₁₀ alkynyl group (preferably a C₂₋₆ alkynyl group etc.) such as ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like, and the like can be mentioned.

As the “alicyclic hydrocarbon group” exemplified for the “aliphatic hydrocarbon group”, for example, a saturated or unsaturated alicyclic hydrocarbon group such as a cycloalkyl group, a cycloalkenyl group, a cycloalkadienyl group and the like can be mentioned.

As used herein, as the “cycloalkyl group”, for example, a C₃₋₁₀ cycloalkyl group (preferably a C₃₋₆ cycloalkyl group etc.) such as cyclopropyl, cyclobutyl, cyclopentyl, cyclohexyl, cycloheptyl, cyclooctyl, cyclononyl, cyclodecyl and the like, and the like can be mentioned.

As the “cycloalkenyl group”, for example, a C₃₋₁₀ cycloalkenyl group (preferably a C₃₋₆ cycloalkenyl group etc.) such as 2-cyclopenten-1-yl, 3-cyclopenten-1-yl, 2-cyclohexen-1-yl, 3-cyclohexen-1-yl, 1-cyclobuten-1-yl, 1-cyclopenten-1-yl, 1-cyclohexen-1-yl, 1-cyclohepten-1-yl and the like, and the like can be mentioned.

As the “cycloalkadienyl group”, for example, a C₄₋₁₀ cycloalkadienyl group (preferably a C₄₋₆ cycloalkadienyl group etc.) such as 2,4-cyclopentadien-1-yl, 2,4-cyclohexadien-1-yl, 2,5-cyclohexadien-1-yl and the like, and the like can be mentioned.

As examples of the “aliphatic hydrocarbon group”, a bi- or tri-cyclic hydrocarbon group derived from a fused ring wherein same or different, two or three rings (preferably two or more kinds of rings) selected from a ring corresponding to the aforementioned alicyclic hydrocarbon group and a ring corresponding to the C₆₋₁₄ aryl group (those exemplified for the below-mentioned “cyclic group” of the “optionally substituted cyclic group” for R₁, R₂, R₃, R₃′, R₄, R₅, R₆, R₇, W₁ or W₂ can be mentioned) are condensed, such as 1,2-dihydronaphthyl, 1,2,3,4-tetrahydronaphthyl, indanyl, indenyl, dihydrobenzocycloheptenyl, fluorenyl and the like, can also be mentioned. In addition, a crosslinked hydrocarbon group such as adamantyl and the like can also be mentioned.

The “aliphatic hydrocarbon group” of the “optionally substituted aliphatic hydrocarbon group” for R or R′ optionally has 1 to 5 (preferably 1 to 3, more preferably 1 or 2) substituents at substitutable positions. When the number of the substituents is not less than 2, respective substituents may be the same or different.

As such substituents, for example,

(i) a nitro group;
(ii) a hydroxy group, an oxo group;
(iii) a cyano group;
(iv) a carbamoyl group;
(v) a mono- or di-C₁₋₆ alkyl-carbamoyl group (e.g., N-methylcarbamoyl, N-ethylcarbamoyl, N,N-dimethylcarbamoyl, N,N-diethylcarbamoyl etc.; the C₁₋₆ alkyl is optionally substituted by a halogen atom, a hydroxy group, a C₁₋₆ alkoxy group and the like), a mono- or di-C₂₋₆ alkenyl-carbamoyl group (e.g., N-allylcarbamoyl etc.; the C₂₋₆ alkenyl is optionally substituted by a halogen atom, a hydroxy group, a C₁₋₆ alkoxy group and the like), a mono- or di-C₆₋₁₂ aryl-carbamoyl group (e.g., mono- or di-phenylcarbamoyl etc.), a mono- or di-aralkyl-carbamoyl group (e.g., a mono- or di-C₇₋₁₀ aralkyl-carbamoyl such as mono- or di-benzylcarbamoyl, mono- or di-phenethylcarbamoyl etc.), a C₁₋₆ alkoxy-carbonyl-carbamoyl group, a C₁₋₆ alkylsulfonyl-carbamoyl group, a C₁₋₆ alkoxy-carbamoyl group, an amino-carbamoyl group, a mono- or di-C₁₋₆ alkylamino-carbamoyl group, a mono- or di-C₆₋₁₂ arylamino-carbamoyl group (e.g., mono- or di-phenylamino-carbamoyl etc.);
(vi) a carboxyl group;
(vii) a C₁₋₆ alkoxy-carbonyl group (e.g., methoxycarbonyl, ethoxycarbonyl, propoxycarbonyl, isopropoxycarbonyl etc.);
(viii) a sulfo group;
(ix) a halogen atom (e.g., fluorine, chlorine, bromine, iodine);
(x) an optionally halogenated C₁₋₆ alkoxy group (e.g., methoxy, ethoxy, propoxy, isopropoxy, trifluoromethoxy etc.), a C₁₋₆ alkoxy group optionally substituted by a hydroxy group and the like, a C₁₋₆ alkoxy group optionally substituted by a carboxyl group and the like, a C₁₋₆ alkoxy group optionally substituted by a C₁₋₆ alkoxy-carbonyl group and the like, a C₁₋₆ alkoxy-C₁₋₆ alkoxy group, a C₁₋₆ alkoxy-C₁₋₆ alkoxy-C₁₋₆ alkoxy group;
(xi) a C₆₋₁₂ aryloxy group, a C₆₋₁₂ aryloxy-C₁₋₆ alkyl group, a C₆₋₁₂ aryl-C₁₋₆ alkoxy group, a C₆₋₁₂ aryloxy-C₁₋₆ alkoxy group, a C₁₋₆ alkyl-carbonyloxy group, a carbamoyloxy group, a mono- or di-C₁₋₆ alkyl-carbamoyloxy group;
(xii) a C₆₋₁₂ aryl group optionally substituted by substituent(s) selected from a halogen atom, a hydroxy group, an optionally halogenated C₁₋₆ alkyl group, an optionally halogenated C₁₋₆ alkoxy group, a C₁₋₆ alkyl group optionally substituted by a hydroxy group and the like, a heterocyclic group (e.g., piperazinyl etc.) optionally substituted by a C₁₋₆ alkyl group and the like, a mono or di-C₁₋₆ alkylamino group, a C₁₋₆ alkanoylamino group and a cyano group;
(xiii) an optionally halogenated C₆₋₁₂ aryl-C₁₋₆ alkyl group, an optionally halogenated. C₆₋₁₂ aryl-C₂₋₆ alkenyl group, an optionally halogenated C₆₋₁₂ aryloxy group (e.g., o-, m- or p-chlorophenoxy, o-, m- or p-bromophenoxy etc.), a pyridyloxy group, a C₃₋₁₀ cycloalkyl-C₁₋₆ alkoxy group, a C₃₋₁₀ cycloalkyl-C₁₋₆ alkyl group;
(xiv) a C₃₋₁₀ cycloalkyl group optionally substituted by a hydroxy group and the like, a bi-cyclic hydrocarbon group (e.g., indanyl etc.) derived from a fused ring wherein a C₃₋₁₀ cycloalkane and a benzene ring are condensed, a crosslinked hydrocarbon group (e.g., adamantyl etc.);
(xv) an optionally halogenated C₁₋₆ alkyl group, an optionally halogenated C₂₋₆ alkenyl group, an optionally halogenated C₁₋₆ alkylthio group (e.g., methylthio, ethylthio, n-propylthio, isopropylthio, n-butylthio etc.), a C₁₋₆ alkyl group optionally substituted by a hydroxy group and the like, a C₁₋₆ alkylthio group optionally substituted by a hydroxy group and the like;
(xvi) a mercapto group, a thioxo group;
(xvii) a benzyloxy group or a benzylthio group, each of which is optionally substituted by substituent(s) selected from a halogen atom, a carboxyl group and a C₁₋₆ alkoxy-carbonyl group;
(xviii) an optionally halogenated C₆₋₁₂ arylthio group, a pyridylthio group, a C₆₋₁₂ arylthio-C₁₋₆ alkyl group, a pyridylthio-C₁₋₆ alkyl group;
(xix) an optionally halogenated C₁₋₆ alkylsulfinyl group (e.g., methylsulfinyl, ethylsulfinyl etc.), a C₆₋₁₂ arylsulfinyl group, a C₆₋₁₂ arylsulfinyl-C₁₋₆ alkyl group;
(xx) an optionally halogenated C₁₋₆ alkylsulfonyl group (e.g., methylsulfonyl, ethylsulfonyl etc.), a C₆₋₁₂ arylsulfonyl group, a C₆₋₁₂ arylsulfonyl-C₁₋₆ alkyl group, a C₆₋₁₂ aryl-C₁₋₆ alkylsulfonyl group;
(xxi) a sulfamoyl group, a mono- or di-C₁₋₁₆ alkylsulfamoyl group (e.g., methylaminosulfonyl, ethylaminosulfonyl, N,N-dimethylaminosulfonyl, N,N-diethylaminosulfonyl etc.; the C₁₋₆ alkyl is optionally substituted by a halogen atom, a hydroxy group, a C₁₋₆ alkoxy group and the like);
(xxii) an amino group, a C₁₋₄ acyl-amino group [for example, a C₁₋₆ alkanoylamino group (e.g., formylamino, acetylamino, trifluoroacetylamino, propionylamino, pivaloylamino etc.), a benzoylamino group, a C₁₋₆ alkylsulfonylamino group (e.g., methanesulfonylamino, trifluoromethanesulfonylamino etc.), a C₆₋₁₄ arylsulfonylamino group (e.g., benzenesulfonylamino, toluenesulfonylamino etc.) etc.; the C₁₋₁₄ acyl is optionally substituted by a halogen atom, a hydroxy group, a carboxyl group and the like], a benzyloxycarbonylamino group, an optionally halogenated C₁₋₆ alkoxy-carbonylamino group, a carbamoylamino group, a mono, or di-C₁₋₆ alkyl-carbamoylamino group;
(xxiii) a mono- or di-C₁₋₆ alkylamino group (e.g., methylamino, ethylamino, dimethylamino, diethylamino, diisopropylamino etc.; the C₁₋₆ alkyl is optionally substituted by a halogen atom, a hydroxy group, a C₁₋₆ alkoxy group and the like), a C₆₋₁₂ arylamino group, a C₆₋₁₂ aryl-C₁₋₆ alkyl-amino group;
(xxiv) a 4- to 6-membered cyclylamino group (e.g., 1-azetidinyl, 1-pyrrolidinyl, piperidino, morpholino, thiomorpholino, 1-piperazinyl, 1,2,3,4-tetrahydroquinolin-1-yl, 1,2,3,4-tetrahydroisoquinolin-2-yl etc.; the cyclylamino group is optionally substituted by a C₁₋₆ alkyl group and the like), a 4- to 6-membered cyclylamino-carbonyl group (e.g., 1-azetidinylcarbonyl, 1-pyrrolidinylcarbonyl, piperidinocarbonyl, morpholinocarbonyl, thiomorpholinocarbonyl, 1-piperazinylcarbonyl, 1,2,3,4-tetrahydroquinolin-1-ylcarbonyl, 1,2,3,4-tetrahydroisoquinolin-2-ylcarbonyl etc.), a 4- to 6-membered cyclylamino-carbonyloxy group (e.g., 1-pyrrolidinylcarbonyloxy, piperidinocarbonyloxy, morpholinocarbonyloxy, thiomorpholinocarbonyloxy, 1-piperazinylcarbonyloxy, 1,2,3,4-tetrahydroquinolin-1-ylcarbonyloxy, 1,2,3,4-tetrahydroisoquinolin-2-ylcarbonyloxy etc.), a 4- to 6-membered cyclylamino-carbonylamino group (e.g., 1-pyrrolidinylcarbonylamino, piperidinocarbonylamino, morpholinocarbonylamino, thiomorpholinocarbonylamino, 1-piperazinylcarbonylamino, 1,2,3,4-tetrahydroquinolin-1-ylcarbonylamino, 1,2,3,4-tetrahydroisoquinolin-2-ylcarbonylamino etc.), a 4- to 6-membered cyclylamino-sulfonyl group (e.g., 1-pyrrolidinylsulfonyl, piperidinosulfonyl, morpholinosulfonyl, thiomorpholinosulfonyl, 1-piperazinylsulfonyl, 1,2,3,4-tetrahydroquinolin-1-ylsulfonyl, 1,2,3,4-tetrahydroisoquinolin-2-ylsulfonyl etc.), a 4- to 6-membered cyclylamino-C₁₋₆ alkyl group;
(xxv) a C₁₋₆ acyl group optionally substituted by substituent(s) selected from a halogen atom, a carboxyl group and a C₁₋₆ alkoxy-carbonyl group (e.g., a optionally halogenated C₁₋₆ alkanoyl group such as formyl, acetyl etc.), a benzoyl group optionally substituted by substituent(s) selected from a halogen atom, a carboxyl group and a C₁₋₆ alkoxy-carbonyl group;
(xxvi) a benzoyl group optionally substituted by a halogen atom and the like;
(xxvii) a 5- to 10-membered heterocyclic group (e.g., 2- or 3-thienyl, 2- or 3-furyl, 2- or 3-pyrrolyl, 3-, 4- or 5-pyrazolyl, 2-, 4- or 5-thiazolyl, 3-, 4- or 5-isothiazolyl, 2-, 4- or 5-oxazolyl, 1,2,3- or 1,2,4-triazolyl, 1H- or 2H-tetrazolyl, 2-, 3- or 4-pyridyl, 2-, 4- or 5-pyrimidyl, 3- or 4-pyridazinyl, pyrazinyl, tetrahydrofuranyl, quinolyl, isoquinolyl, indolyl, dihydrobenzoxazinyl, benzodioxoly, a 5- or 6-membered cyclylamino group included in the “4- to 6-membered cyclylamino group” recited in the aforementioned (xxiv) and the like can be mentioned; the heterocyclic group is optionally substituted by a C₁₋₆ alkyl group (the C₁₋₆ alkyl group is optionally substituted by a hydroxy group and the like), a C₁₋₆ alkoxy-carbonyl group, a C₁₋₆ alkylthio group, a C₁₋₆ alkoxy group, an amino group and the like);
(xxviii) a 5- to 10-membered heterocyclyl-carbonyl group (e.g., 2- or 3-thienylcarbonyl, 2- or 3-furylcarbonyl, 3-, 4- or 5-pyrazolylcarbonyl, 2-, 4- or 5-thiazolylcarbonyl, 3-, 4- or 5-isothiazolylcarbonyl, 2-, 4- or 5-oxazolylcarbonyl, 1,2,3- or 1,2,4-triazolylcarbonyl, 1H- or 2H-tetrazolylcarbonyl, 2-, 3- or 4-pyridylcarbonyl, 2-, 4- or 5-pyrimidylcarbonyl, 3- or 4-pyridazinylcarbonyl, quinolylcarbonyl, isoquinolylcarbonyl, indolylcarbonyl etc.; the heterocyclic group is optionally substituted by a C₁₋₆ alkyl group and the like);
(xxix) a hydroxyimino group, a C₁₋₆ alkoxyimino group;
(xxx) an optionally halogenated linear or branched C₁₋₄ alkylenedioxy group (e.g., methylenedioxy, ethylenedioxy, propylenedioxy, tetrafluoroethylenedioxy etc.); and the like can be mentioned.

The “C₁₋₆ alkyl” exemplified for the substituents which the aforementioned “aliphatic hydrocarbon group” optionally has, may be linear or branched and, for example, methyl, ethyl, n-propyl, isopropyl, n-butyl, isobutyl, sec-butyl, tert-butyl, n-pentyl, isopentyl, neopentyl, 1-methylpropyl, n-hexyl, isohexyl, 1,1-dimethylbutyl, 2,2-dimethylbutyl, 3,3-dimethylbutyl, 3,3-dimethylpropyl, 2-ethylbutyl and the like can be mentioned.

The “C₂₋₆ alkenyl” exemplified for the substituents which the aforementioned “aliphatic hydrocarbon group” optionally has, may be linear or branched and, for example, vinyl, allyl, isopropenyl, 2-methylallyl, 1-propenyl, 2-methyl-1-propenyl, 1-butenyl, 2-butenyl, 3-butenyl, 2-ethyl-1-butenyl, 2-methyl-2-butenyl, 3-methyl-2-butenyl, 1-pentenyl, 2-pentenyl, 3-pentenyl, 4-pentenyl, 4-methyl-3-pentenyl, 1-hexenyl and the like can be mentioned.

As the “C₃₋₁₀ cycloalkyl” exemplified for the substituents which the aforementioned “aliphatic hydrocarbon group” optionally has, those exemplified for the aforementioned “aliphatic hydrocarbon group” can be mentioned.

As the “C₆₋₁₂ aryl” exemplified for the substituents which the aforementioned “aliphatic hydrocarbon group” optionally has, for example, phenyl, 1-naphthyl, 2-naphthyl, 2-biphenylyl, 3-biphenylyl, 4-biphenylyl and the like can be mentioned.

As the “C₁₋₆ alkoxy” exemplified for the substituents which the aforementioned “aliphatic hydrocarbon group” optionally has, for example, methoxy, ethoxy, propoxy, isopropoxy, butoxy, isobutoxy, tert-butoxy, pentyloxy, hexyloxy and the like can be mentioned.

As the “C₇₋₁₀ aralkyl” exemplified for the substituents which the aforementioned “aliphatic hydrocarbon group” optionally has, for example, benzyl, 1-phenylethyl, 2-phenylethyl, 3-phenylpropyl, 4-phenylbutyl and the like (preferably a phenyl-C₁₋₄ alkyl group etc.) can be mentioned.

As the “C₁₋₆ alkanoyl” exemplified for the substituents which the aforementioned “aliphatic hydrocarbon group” optionally has, for example, formyl, acetyl, propionyl, butyryl, pivaloyl and the like can be mentioned.

As the “aliphatic chain hydrocarbon group” of the “optionally substituted aliphatic chain hydrocarbon group” for R₁, R₂, R₃, R₃′, R₄, R₅, R₆, R₇, W₁ or W₂, those exemplified for the aforementioned “aliphatic hydrocarbon group” of the “optionally substituted aliphatic hydrocarbon group” for R or R′ can be mentioned.

The “aliphatic chain hydrocarbon group” of the “optionally substituted aliphatic chain hydrocarbon group” for R₁, R₂, R₃, R₃′, R₄, R₅, R₆, R₇, W₁ or W₂ optionally has 1 to 5 (preferably 1 to 3, more preferably 1 or 2) substituents at substitutable positions. When the number of the substituents is not less than 2, respective substituents may be the same or different.

As such substituents, for example, those similar to the substituents which the aforementioned “aliphatic hydrocarbon group” of the “optionally substituted aliphatic hydrocarbon group” for R or R′ optionally has, and the like can be mentioned.

As the “cyclic group” of the “optionally substituted cyclic group” for R₁, R₂, R₃, R₃′, R₄, R₅, R₆, R₇, W₁ or W₂, for example, an aromatic group, a non-aromatic cyclic group and the like can be mentioned.

As the “aromatic group” exemplified for the “cyclic group”, for example, an aromatic hydrocarbon group, an aromatic heterocyclic group and the like can be mentioned.

As used herein, as the “aromatic hydrocarbon group”, for example, a C₆₋₁₄ aryl group (preferably a C₆₋₁₂ aryl group) such as phenyl, 1-naphthyl, 2-naphthyl, 2-biphenylyl, 3-biphenylyl, 4-biphenylyl, 1-anthracenyl, 1-phenanthrenyl, 1-acenaphthylenyl and the like, and the like can be mentioned.

As the “aromatic heterocyclic group”, for example, a 3- to 8-membered (preferably 4- to 7-membered, more preferably 5- or 6-membered) monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group can be mentioned. As the fused aromatic heterocyclic group, for example, a group derived from a fused ring wherein a ring corresponding to the 3- to 8-membered monocyclic aromatic heterocyclic group, and 1 or 2 rings selected from a 5- or 6-membered aromatic heterocycle containing 1 or 2 nitrogen atoms, a 5-membered aromatic heterocycle containing one sulfur atom and a benzene ring are condensed, and the like can be mentioned.

As preferable examples of the “aromatic heterocyclic group”,

a monocyclic aromatic heterocyclic group such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 3-isothiazolyl, 4-isothiazolyl, 5-isothiazolyl), oxazolyl (e.g., 2-oxazolyl, 4-oxazolyl, 5-oxazolyl), isoxazolyl (e.g., 3-isoxazolyl, 4-isoxazolyl, 5-isoxazolyl), oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl); triazinyl (e.g., 1,2,4-triazin-1-yl, 1,2,4-triazin-3-yl) and the like;
a fused aromatic heterocyclic group such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuranyl (e.g., 2-benzofuranyl, 3-benzofuranyl), benzothienyl (e.g., 2-benzothienyl, 3-benzothienyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like;
and the like can be mentioned.

As the “non-aromatic cyclic group” exemplified for the “cyclic group”, for example, a non-aromatic cyclic hydrocarbon group, a non-aromatic heterocyclic group and the like can be mentioned.

As used herein, as the “non-aromatic cyclic hydrocarbon group”, for example, a cycloalkyl group, a cycloalkenyl group and a cycloalkadienyl group, each of which is optionally condensed with a benzene ring, and the like can be mentioned. As the “cycloalkyl group”, “cycloalkenyl group” and “cycloalkadienyl group”, those exemplified for the aforementioned “aliphatic hydrocarbon group” the “optionally substituted aliphatic hydrocarbon group” for R or R′ can be mentioned.

As the “non-aromatic heterocyclic group”, for example, a 3- to 8-membered (preferably 4- to 7-membered, more preferably 5- or 6-membered) monocyclic non-aromatic heterocyclic group containing, as a ring-constituting atom besides carbon atoms, 1 to 4 heteroatoms selected from an oxygen atom, a sulfur atom and a nitrogen atom, and a fused non-aromatic heterocyclic group can be mentioned. As the fused non-aromatic heterocyclic group, for example, a group derived from a fused ring wherein a ring corresponding to the 3- to 8-membered monocyclic non-aromatic heterocyclic group, and 1 or 2 heterocyclic rings selected from a 5- or 6-membered heterocyclic ring containing 1 or 2 nitrogen atoms, a 5-membered ring containing one sulfur atom and a benzene ring are condensed, and the like can be mentioned.

As preferable examples of “non-aromatic heterocyclic group”,

a monocyclic non-aromatic heterocyclic group such as aziridinyl (e.g., 1-aziridinyl, 2-aziridinyl), azetidinyl (e.g., 1-azetidinyl, 2-azetidinyl, 3-azetidinyl), pyrrolidinyl (e.g., 1-pyrrolidinyl, 2-pyrrolidinyl, 3-pyrrolidinyl), piperidinyl (e.g., piperidino, 2-piperidinyl, 3-piperidinyl, 4-piperidinyl), morpholinyl (e.g., morpholino), thiomorpholinyl (e.g., thiomorpholino), piperazinyl (e.g., 1-piperazinyl, 2-piperazinyl, 3-piperazinyl), hexamethyleniminyl (e.g., hexamethylenimin-1-yl), oxazolidinyl (e.g., oxazblidin-2-yl), thiazolidinyl (e.g., thiazolidin-2-yl), imidazolidinyl (e.g., imidazolidin-2-yl, imidazolidin-3-yl), oxazolinyl (e.g., oxazolin-2-yl), thiazolinyl (e.g., thiazolin-2-yl), imidazolinyl (e.g., imidazolin-2-yl, imidazolin-3-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), 2-thioxo-1,3-oxazolidin-5-yl, pyranyl (e.g., 4-pyranyl), tetrahydropyranyl (e.g., 2-tetrahydropyranyl, 3-tetrahydropyranyl, 4-tetrahydropyranyl), thiopyranyl (e.g., 4-thiopyranyl), tetrahydrothiopyranyl (e.g., 2-tetrahydrothiopyranyl, 3-tetrahydrothiopyranyl, 4-tetrahydrothiopyranyl), 1-oxidetetrahydrothiopyranyl (e.g., 1-oxidetetrahydrothiopyran-4-yl), 1,1-dioxidetetrahydrothiopyranyl (e.g., 1,1-dioxidetetrahydrothiopyran-4-yl), tetrahydrofuryl (e.g., tetrahydrofuran-3-yl, tetrahydrofuran-2-yl), pyrazolidinyl (e.g., pyrazolidin-1-yl, pyrazolidin-3-yl), pyrazolinyl (e.g., pyrazolin-1-yl), tetrahydropyrimidinyl (e.g., tetrahydropyrimidin-1-yl), dihydrotriazolyl (e.g., 2,3-dihydro-1H-1,2,3-triazol-1-yl), tetrahydrotriazolyl (e.g., 2,3,4,5-tetrahydro-1H-1,2,3-triazol-1-yl) and the like; a fused non-aromatic heterocyclic group such as dihydroindolyl (e.g., 2,3-dihydro-1H-indol-1-yl), dihydroisoindolyl (e.g., 1,3-dihydro-2H-isoindol-2-yl), dihydrobenzofuranyl (e.g., 2,3-dihydrobenzofuran-5-yl), dihydrobenzodioxynyl (e.g., 2,3-dihydro-1,4-benzodioxynyl), dihydrobenzodioxepinyl (e.g., 3,4-dihydro-2H-1,5-benzodioxepinyl), tetrahydrobenzofuranyl (e.g., 4,5,6,7-tetrahydrobenzofuran-3-yl), chromenyl (e.g., 4H-chromen-2-yl, 2H-chromen-3-yl), dihydroquinolinyl (e.g., 1,2-dihydroquinolin-4-yl), tetrahydroquinolinyl (e.g., 1,2,3,4-tetrahydroquinolin-4-yl), dihydroisoquinolinyl (e.g., 1,2-dihydroisoquinolin-4-yl), tetrahydroisoquinolinyl (e.g., 1,2,3,4-tetrahydroisoquinolin-4-yl), dihydrophthalazinyl (e.g., 1,4-dihydrophthalazin-4-yl) and the like;
and the like can be mentioned.

The “cyclic group” of the “optionally substituted cyclic group” for R₁, R₂, R₃, R₃′, R₄, R₅, R₆, R₇, W₁ or W₂ optionally has 1 to 5 (preferably 1 to 3, more preferably 1 or 2) substituents at substitutable positions. When the number of the substituents is not less than 2, respective substituents may be the same or different.

As such substituents, for example, those similar to the substituents which the aforementioned “aliphatic hydrocarbon group” of the “optionally substituted aliphatic hydrocarbon group” for R or R′ optionally has, and the like can be mentioned.

As the “optionally substituted carbamoyl group” for R, R′, R₁, R₂, R₃, R₃′, R₄, R₅, R₆, R₇, W₁ or W₂, a unsubstituted carbamoyl group, a N-mono-substituted carbamoyl group and a N,N-di-substituted carbamoyl group can be mentioned.

As the substituent of the “N-mono-substituted carbamoyl group”, for example, an optionally substituted hydrocarbon group, an optionally substituted heterocyclic group and the like can be mentioned.

As the “hydrocarbon group” of the “optionally substituted hydrocarbon group” exemplified as the substituent of the “N-mono-substituted carbamoyl group”, for example, an aliphatic hydrocarbon group, an aryl group (an aromatic hydrocarbon group) and the like can be mentioned.

As used herein, as the “aliphatic hydrocarbon group”, those similar to the aforementioned “aliphatic hydrocarbon group” of the “optionally substituted aliphatic hydrocarbon group” for R or R′ can be mentioned.

As the “aryl group (aromatic hydrocarbon group)”, those exemplified for the aforementioned “cyclic group” of the “optionally substituted cyclic group” for R₁, R₂, R₃, R₃′, R₄, R₅, R₆, R₇, W₁ or W₂ can be mentioned.

The “hydrocarbon group” of the “optionally substituted hydrocarbon group” exemplified as the substituent of the “N-mono-substituted carbamoyl group” optionally has 1 to 5 (preferably 1 to 3, more preferably 1 or 2) substituents at substitutable positions. When the number of the substituents is not less than 2, respective substituents may be the same or different.

As such substituents, for example, those similar to the substituents which the aforementioned “aliphatic hydrocarbon group” of the “optionally substituted aliphatic hydrocarbon group” for R or R′ optionally has, and the like can be mentioned.

As the “heterocyclic group” of the “optionally substituted heterocyclic group” exemplified as the substituent of the “N-mono-substituted carbamoyl group”, for example, an aromatic heterocyclic group, a non-aromatic heterocyclic group and the like can be mentioned.

As used herein, as the “aromatic heterocyclic group” and “non-aromatic heterocyclic group”, those exemplified for the aforementioned “cyclic group” of the “optionally substituted cyclic group” for R₁, R₂, R₃, R₃′, R₄, R₅, R₆, R₇, W₁ or W₂ can be mentioned.

The “heterocyclic group” of the “optionally substituted heterocyclic group” exemplified as the substituent of the “N-mono-substituted carbamoyl group” optionally has 1 to 5 (preferably 1 to 3, more preferably 1 or 2) substituents at substitutable positions. When the number of the substituents is not less than 2, respective substituents may be the same or different.

As such substituents, for example, those similar to the substituents which the aforementioned “aliphatic hydrocarbon group” of the “optionally substituted aliphatic hydrocarbon group” for R or R′ optionally has, and the like can be mentioned.

The “N,N-di-substituted carbamoyl group” means a carbamoyl group having two substituents at the nitrogen atom. As examples of one of the two substituents, those similar to the substituents for the above-mentioned “N-mono-substituted carbamoyl group” can be mentioned, and as examples of the other substituent, for example, a C₁₋₆ alkyl group (e.g., methyl, ethyl, propyl, isopropyl, butyl, tert-butyl, pentyl, hexyl etc.), a C₃₋₆ cycloalkyl group (e.g., cyclopropyl, cyclobutyl, cyclopentyl, cyclohexyl etc.), a C₇₋₁₀ aralkyl group (e.g., benzyl, phenethyl, phenylpropyl, phenylbutyl etc., preferably a phenyl-C₁₋₄ alkyl group etc.) and the like can be mentioned. The two substituents in combination may form a cyclylamino group together with the nitrogen atom. As the cyclylamino-carbonyl group in this case, for example, a 3- to 8-membered (preferably 5- or 6-membered) cyclylamino-carbonyl group such as 1-azetidinylcarbonyl, 1-pyrrolidinylcarbonyl, piperidinocarbonyl, morpholinocarbonyl, thiomorpholinocarbonyl (the sulfur atom is optionally oxidized), 1-piperazinylcarbonyl, 1-piperazinylcarbonyl optionally having, at the 4-position, a C₁₋₆ alkyl group (e.g., methyl, ethyl, propyl, isopropyl, butyl, tert-butyl, pentyl, hexyl etc.), a C₇₋₁₀ aralkyl group (e.g., benzyl, phenethyl, phenylpropyl, phenylbutyl etc., preferably a phenyl-C₁₋₄ alkyl group etc.), a C₆₋₁₀ aryl group (e.g., phenyl, 1-naphthyl, 2-naphthyl etc.) and the like, and the like can be mentioned.

As the “optionally esterified carboxyl group” for R, R′, R₁, R₂, R₃, R₃′, R₄, R₅, R₆, R₇, W₁ or W₂, a free carboxyl group, a lower alkoxycarbonyl group, an aryloxycarbonyl group, an aralkyloxycarbonyl group and the like can be mentioned.

As the “lower alkoxycarbonyl group”, for example, a C₁₋₆ alkoxy-carbonyl group such as methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, isopentyloxycarbonyl, neopentyloxycarbonyl and the like, and the like can be mentioned. Of these, a C₁₋₃ alkoxy-carbonyl group such as methoxycarbonyl, ethoxycarbonyl, propoxycarbonyl and the like, and the like are preferable.

As the “aryloxycarbonyl group”, for example, a C₆₋₁₄ aryl-oxycarbonyl group such as phenoxycarbonyl, 1-naphthoxycarbonyl, 2-naphthoxycarbonyl and the like, and the like can be mentioned. Of these, a C₆₋₁₂ aryl-oxycarbonyl group and the like are preferable.

As the “aralkyloxycarbonyl group”, for example, a C₇₋₁₄ aralkyl-oxycarbonyl group such as benzyloxycarbonyl, phenethyloxycarbonyl and the like, and the like can be mentioned. Of these, a C₆₋₁₀ aryl-C₁₋₄ alkoxy-carbonyl group and the like are preferable.

The “lower alkoxycarbonyl group”, “aryloxycarbonyl group” and “aralkyloxycarbonyl group” optionally have 1 to 5 (preferably 1 to 3, more preferably 1 or 2) substituents at substitutable positions. When the number of the substituents is not less than 2, respective substituents may be the same or different.

As such substituents, for example, those similar to the substituents which the aforementioned “aliphatic hydrocarbon group” of the “optionally substituted aliphatic hydrocarbon group” for R or R′ optionally has, and the like can be mentioned.

As the “acyl group” for R, R′, R₁, R₂, R₃, R₃′, R₄, R₅, R₆, R₇, W₁ or W₂, an acyl group derived from carboxylic acid, an acyl group derived from sulfinic acid, an acyl group derived from sulfonic acid, an acyl group derived from phosphonic acid and the like can be mentioned.

As the “acyl group derived from carboxylic acid”, a group wherein carbonyl (—C(O)—) is bonded to a hydrogen atom, an optionally substituted hydrocarbon group (e.g., those similar to the aforementioned “optionally substituted hydrocarbon group” exemplified as the substituent of the “optionally substituted carbamoyl group” for R, R′, R₁, R₂, R₃, R₃′, R₄, R₅, R₆, R₇, W₁ or W₂, and the like) or an optionally substituted heterocyclic group (e.g., those similar to the aforementioned “optionally substituted heterocyclic group” exemplified as the substituent of the “optionally substituted carbamoyl group” for R, R′, R₁, R₂, R₃, R₃′, R₄, R₅, R₆, R₇, W₁ or W₂, and the like) can be mentioned. As preferable examples, formyl, an optionally substituted alkylcarbonyl group (as the alkylcarbonyl group, for example, a C₁₋₁₀ alkyl-carbonyl group such as acetyl, propionyl, butyryl, isobutyryl, valeryl, isovaleryl, pivaloyl, hexanoyl and the like, and the like can be mentioned), an optionally substituted cycloalkylcarbonyl group (as the cycloalkylcarbonyl group, for example, a C₃₋₁₀ cycloalkyl-carbonyl group such as cyclobutanecarbonyl, cyclopentanecarbonyl, cyclohexanecarbonyl and the like, and the like can be mentioned), an optionally substituted arylcarbonyl group (as the arylcarbonyl group, for example, a C₆₋₁₄ aryl-carbonyl group such as benzoyl, naphthoyl and the like, and the like can be mentioned), an optionally substituted aromatic heterocyclyl-carbonyl group (as the aromatic heterocyclyl-carbonyl group, for example, a 5- or 6-membered aromatic heterocyclyl-carbonyl group such as pyridylcarbonyl, pyrazolylcarbonyl, imidazolylcarbonyl, oxazolylcarbonyl, isoxazolylcarbonyl, thiazolylcarbonyl and the like, and the like can be mentioned) and the like can be mentioned. Of these, a C₁₋₆ alkyl-carbonyl group, a C₃₋₆ cycloalkyl-carbonyl group, a C₆₋₁₂ aryl-carbonyl group and the like are preferable.

As the “acyl group derived from sulfinic acid”, a group wherein sulfinyl (—S(O)—) is bonded to a hydrogen atom, an optionally substituted hydrocarbon group (e.g., those similar to the aforementioned “optionally substituted hydrocarbon group” exemplified as the substituent of the “optionally substituted carbamoyl group” for R, R′, R₁, R₂, R₃, R₃′, R₄, R₅, R₆, R₇, W₁ or W₂, and the like) or an optionally substituted heterocyclic group (e.g., those similar to the aforementioned “optionally substituted heterocyclic group” exemplified as the substituent of the “optionally substituted carbamoyl group” for R, R′, R₁, R₂, R₃, R₃′, R₄, R₅, R₆, R₇, W₁ or W₂, and the like) can be mentioned. As preferable examples, an optionally substituted alkylsulfinyl group (as the alkylsulfinyl group, for example, a C₁₋₁₀ alkylsulfinyl group such as methanesulfinyl, ethanesulfinyl, propanesulfinyl and the like, and the like can be mentioned), an optionally substituted cycloalkylsulfinyl group (as the cycloalkylsulfinyl group, for example, a C₃₋₁₀ cycloalkylsulfinyl group such as cyclopropanesulfinyl, cyclopentanesulfinyl, cyclohexanesulfinyl and the like, and the like can be mentioned), an optionally substituted arylsulfinyl group (as the arylsulfinyl group, for example, a C₆₋₁₄ arylsulfinyl group such as benzenesulfinyl, naphthalenesulfinyl and the like, and the like can be mentioned), an optionally substituted aromatic heterocyclyl-sulfinyl group (as the aromatic heterocyclyl-sulfinyl group, for example, a 5- or 6-membered aromatic heterocyclyl-sulfinyl group such as pyridylsulfinyl, pyrazolylsulfinyl, imidazolylsulfinyl, oxazolylsulfinyl, isoxazolylsulfinyl, thiazolylsulfinyl and the like, and the like can be mentioned) and the like can be mentioned. Of these, a C₁₋₆ alkylsulfinyl group, a C₃₋₆ cycloalkylsulfinyl group, a C₆₋₁₂ arylsulfinyl group and the like are preferable.

As the “acyl group derived from sulfonic acid”, a group wherein sulfonyl (—S(O)₂—) is bonded to a hydrogen atom, an optionally substituted hydrocarbon group (e.g., those similar to the aforementioned “optionally substituted hydrocarbon group” exemplified as the substituent of the “optionally substituted carbamoyl group” for R, R′, R₁, R₂, R₃, R₃′, R₄, R₅, R₆, R₇, W₁ or W₂, and the like) or an optionally substituted heterocyclic group (e.g., those similar to the aforementioned “optionally substituted heterocyclic group” exemplified as the substituent of the “optionally substituted carbamoyl group” for R, R′, R₁, R₂, R₃, R₃′, R₄, R₅, R₆, R₇, W₁ or W₂, and the like) can be mentioned. As preferable examples, an optionally substituted alkylsulfonyl group (as the alkylsulfonyl group, for example, a C₁₋₁₀ alkylsulfonyl group such as methanesulfonyl, ethanesulfonyl, propanesulfonyl and the like, and the like can be mentioned), an optionally substituted cycloalkylsulfonyl group (as the cycloalkylsulfonyl group, for example, a C₃₋₁₀ cycloalkylsulfonyl group such as cyclopropanesulfonyl, cyclopentanesulfonyl, cyclohexanesulfonyl and the like, and the like can be mentioned), an optionally substituted arylsulfonyl group (as the arylsulfonyl group, for example, a C₆₋₁₄ arylsulfonyl group such as benzenesulfonyl, naphthalenesulfonyl and the like, and the like can be mentioned), an optionally substituted aromatic heterocyclyl-sulfonyl group (as the aromatic heterocyclyl-sulfonyl group, for example, a 5- or 6-membered aromatic heterocyclyl-sulfonyl group such as pyridylsulfonyl, pyrazolylsulfonyl, imidazolylsulfonyl, oxazolylsulfonyl, isoxazolylsulfonyl, thiazolylsulfonyl and the like, and the like can be mentioned) and the like can be mentioned. Of these, a C₁₋₆ alkylsulfonyl group, a C₃₋₆ cycloalkylsulfonyl group, a C₆₋₁₂ arylsulfonyl group and the like are preferable.

As the “acyl group derived from phosphonic acid”, for example, a mono- or di-C₁₋₆ alkylphosphono group optionally forming a ring, such as dimethylphosphono, diethylphosphono, diisopropylphosphono, dibutylphosphono, 2-oxido-1,3,2-dioxaphosphinan-2-yl and the like, and the like can be mentioned.

The “hydroxy group”, “mercapto group” and “amino group” of the “optionally substituted hydroxy group”, “optionally substituted mercapto group” and “optionally substituted amino group” for R, R′, R₁, R₂, R₃, R₃′, R₄, R₅, R₆, R₇, W₁ or W₂, and the “imino group (═NH)” of the “optionally substituted imino group” for R₃ or R₃′ optionally have substituent(s) at substitutable positions. When the number of the substituents is not less than 2, respective substituents may be the same or different.

As such substituents, for example,

(i) an optionally substituted hydrocarbon group (e.g., those similar to the aforementioned “optionally substituted hydrocarbon group” exemplified as the substituent of the “optionally substituted carbamoyl group” for R, R′, R₁, R₂, R₃, R₃′, R₄, R₅, R₆, R₇, W₁ or W₂, and the like);
(ii) an acyl group (e.g., those similar to the aforementioned “acyl group” for R, R′, R₁, R₂, R₃, R₃′, R₄, R₅, R₆, R₇, W₁ or W₂, and the like);
(iii) an optionally esterified carboxyl group (e.g., those similar to the aforementioned “optionally esterified carboxyl group” for R, R′, R₁, R₂, R₃, R₃′, R₄, R₅, R₆, R₇, W₁ or W₂, and the like);
(iv) an optionally substituted carbamoyl group (e.g., those similar to the aforementioned “optionally substituted carbamoyl group” for R, R′, R₁, R₂, R₃, R₃′, R₄, R₅, R₆, R₇, W₁ or W₂, and the like);
(v) an optionally substituted heterocyclic group (e.g., those similar to the aforementioned “optionally substituted heterocyclic group” exemplified as the substituent of the “optionally substituted carbamoyl group” for R, R′, R₁, R₂, R₃, R₃′, R₄, R₅, R₆, R₇, W₁ or W₂, and the like);
and the like can be mentioned.

As preferable examples of the “optionally substituted hydroxy group”, an optionally substituted alkoxy group (as the alkoxy group, for example, a C₁₋₁₀ alkoxy group such as methoxy, ethoxy, propoxy and the like, and the like can be mentioned), an optionally substituted cycloalkoxy group (as the cycloalkoxy group, for example, a C₃₋₁₀ cycloalkoxy group such as cyclopropoxy, cyclopentyloxy, cyclohexyloxy and the like, and the like can be mentioned), an optionally substituted aryloxy group (as the aryloxy group, for example, a C₆₋₁₄ aryloxy group such as phenyloxy, naphthyloxy and the like, and the like can be mentioned), an optionally substituted aromatic heterocyclyl-oxy group (as the aromatic heterocyclyl-oxy group, for example, a 5- or 6-membered aromatic heterocyclyl-oxy group such as pyridyloxy, pyrazolyloxy, imidazolyloxy, oxazolyloxy, isoxazolyloxy, thiazolyloxy and the like, and the like can be mentioned) and the like can be mentioned. Of these, a C₁₋₆ alkyloxy group, a C₃₋₆ cycloalkyloxy group, a C₆₋₁₂ aryloxy group and the like are preferable.

As preferable examples of the “optionally substituted mercapto group”, an optionally substituted alkylthio group (as the alkylthio group, for example, a C₁₋₁₀ alkylthio group such as methylthio, ethylthio, propylthio and the like, and the like can be mentioned), an optionally substituted cycloalkylthio group (as the cycloalkylthio group, for example, a C₃₋₁₀ cycloalkylthio group such as cyclopropylthio, cyclopentylthio, cyclohexylthio and the like, and the like can be mentioned), an optionally substituted arylthio group (as the arylthio group, for example, a C₆₋₁₄ arylthio group such as phenylthio, naphthylthio and the like, and the like can be mentioned), an optionally substituted aromatic heterocyclyl-thio group (as the aromatic heterocyclyl-thio group, for example, a 5- or 6-membered aromatic heterocyclyl-thio group such as pyridylthio, pyrazolylthio, imidazolylthio, oxazolylthio, isoxazolylthio, thiazolylthio and the like, and the like can be mentioned) and the like can be mentioned. Of these, a C₁₋₆ alkylthio group, a C₃₋₆ cycloalkylthio group, a C₆₋₁₂ arylthio group and the like are preferable.

As preferable examples of the “optionally substituted amino group”, an optionally substituted mono or di-alkylamino group (as the mono or di-alkylamino group, for example, a mono or di-C₁₋₁₀ alkylamino group such as methylamino, ethylamino, propylamino, dimethylamino, diethylamino, dipropylamino and the like, and the like can be mentioned), an optionally substituted mono or di-cycloalkylamino group (as the mono or di-cycloalkylamino group, for example, a mono or di-C₃₋₁₀ cycloalkylamino group such as cyclopropylamino, cyclopentylamino, cyclohexylamino, dicyclopropylamino, dicyclopentylamino, dicyclohexylamino and the like, and the like can be mentioned), an optionally substituted mono or di-arylamino group (as the mono or di-arylamino group, for example, a mono or di-C₆₋₁₄ arylamino group such as phenylamino, naphthylamino, diphenylamino, dinaphthylamino and the like, and the like can be mentioned), an optionally substituted mono or di-aromatic heterocyclyl-amino group (as the mono or di-aromatic heterocyclyl-amino group, for example, a mono or di-5- or 6-membered aromatic heterocyclyl-amino group such as mono or di-pyridylamino, mono or di-pyrazolylamino, mono or di-imidazolylamino, mono or di-oxazolylamino, mono or di-isoxazolylamino, mono or di-thiazolylamino and the like, and the like can be mentioned) and the like can be mentioned. Of these, a mono or di-C₁₋₆ alkylamino group, a mono or di-C₃₋₆ cycloalkylamino group, a mono or di-C₆₋₁₂ arylamino group and the like are preferable.

As preferable examples of the “optionally substituted imino group”, an optionally substituted alkylimino group (as the alkylimino group, for example, a C₁₋₁₀ alkylimino group such as methylimino, ethylimino, propylimino and the like, and the like can be mentioned), an optionally substituted cycloalkylimino group (as the cycloalkylimino group, for example, a C₃₋₁₀ cycloalkylimino group such as cyclopropylimino, cyclopentylimino, cyclohexylimino and the like, and the like can be mentioned), an optionally substituted arylimino group (as the arylimino group, for example, a C₆₋₁₄ arylimino group such as phenylimino, naphthylimino and the like, and the like can be mentioned), an optionally substituted aromatic heterocyclyl-imino group (as the aromatic heterocyclyl-imino group, for example, a 5- or 6-membered aromatic heterocyclyl-imino group such as pyridylimino, pyrazolylimino, imidazolylimino, oxazolylimino, isoxazolylimino, thiazolylimino and the like, and the like can be mentioned) and the like can be mentioned. Of these, a C₁₋₆ alkylimino group, a C₃₋₆ cycloalkylimino group, a C₆₋₁₂ arylimino group and the like are preferable.

In addition, the “amino group” of the “optionally substituted amino group” and the “imino group” of the “optionally substituted imino group” are optionally substituted by an optionally substituted imidoyl group (e.g., a C₁₋₆ alkylimidoyl group (e.g., formylimidoyl, acetylimidoyl etc.), a C₁₋₆ alkoxyimidoyl group, a C₁₋₆ alkylthioimidoyl group, an amidino group etc.), an amino group optionally substituted by 1 or 2 C₁₋₆ alkyl groups and the like.

When the “amino group” of the “optionally substituted amino group” is substituted by two substituents, the two substituents may be the same or different.

In addition, the two substituents of the “optionally substituted amino group” in combination may form, together with the nitrogen atom, a cyclylamino group. As the cyclylamino group in this case, for example, a 3- to 8-membered (preferably 5- or 6-membered) cyclylamino group such as 1-azetidinyl, 1-pyrrolidinyl, piperidino, thiomorpholino, morpholino, 1-piperazinyl, and 1-piperazinyl, 1-pyrrolyl and 1-imidazolyl, each optionally having, at the 4-position, a C₁₋₆ alkyl group (e.g., methyl, ethyl, propyl, isopropyl, butyl, tert-butyl, pentyl, hexyl etc.), a C₇₋₁₀ aralkyl group (e.g., benzyl, phenethyl, phenylpropyl, phenylbutyl etc., preferably phenyl-C₁₋₄ alkyl group etc.), a C₆₋₁₀ aryl group (e.g., phenyl, 1-naphthyl, 2-naphthyl etc.) and the like, and the like can be mentioned.

With regard to the “oxo group” for R₃ or R₃′ and the “imino group” of the “optionally substituted imino group” for R₃ or R₃′, two R₃ bonded to a single carbon atom form an oxo group in combination, and two R₃′ bonded to a single carbon atom form an imino group in combination.

As the “spiro ring” formed by two R together with the carbon atom they are bonded to, a ring wherein a non-aromatic ring and

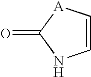

are bonded to commonly have a single carbon atom can be mentioned.

As the “non-aromatic ring”, an alicyclic hydrocarbon, a non-aromatic heterocycle and the like can be mentioned.

As the “alicyclic hydrocarbon” exemplified for the “non-aromatic ring”, a ring corresponding to the aforementioned “alicyclic hydrocarbon group” exemplified for the “aliphatic hydrocarbon group” of the “optionally substituted aliphatic hydrocarbon group” for R or R′ can be mentioned. Specifically, a C₃₋₁₀ cycloalkane, a C₃₋₁₀ cycloalkene, a C₄₋₁₀ cycloalkadiene and the like can be mentioned.

As the “non-aromatic heterocycle” exemplified for the “non-aromatic ring”, a ring corresponding to the aforementioned “non-aromatic heterocyclic group” exemplified for the “cyclic group” of the “optionally substituted cyclic group” for R₁, R₂, R₃, R₃′, R₄, R₅, R₆, R₇, W₁ or W₂ can be mentioned. Specifically, aziridine, azetidine, pyrrolidine, piperidine, morpholine, thiomorpholine, piperazine, hexamethylenimine, oxazolidine, thiazolidine, imidazolidine, oxazoline, thiazoline, imidazoline, dioxole, dioxolane, dihydrooxadiazole, pyran, tetrahydropyran, thiopyran, tetrahydrothiopyran, tetrahydrofuran, pyrazolidine, pyrazoline, tetrahydropyrimidine, dihydrotriazole, tetrahydrotriazole

and the like can be mentioned.

The group represented by the formula:

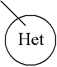

is a heterocyclic group represented by the formula:

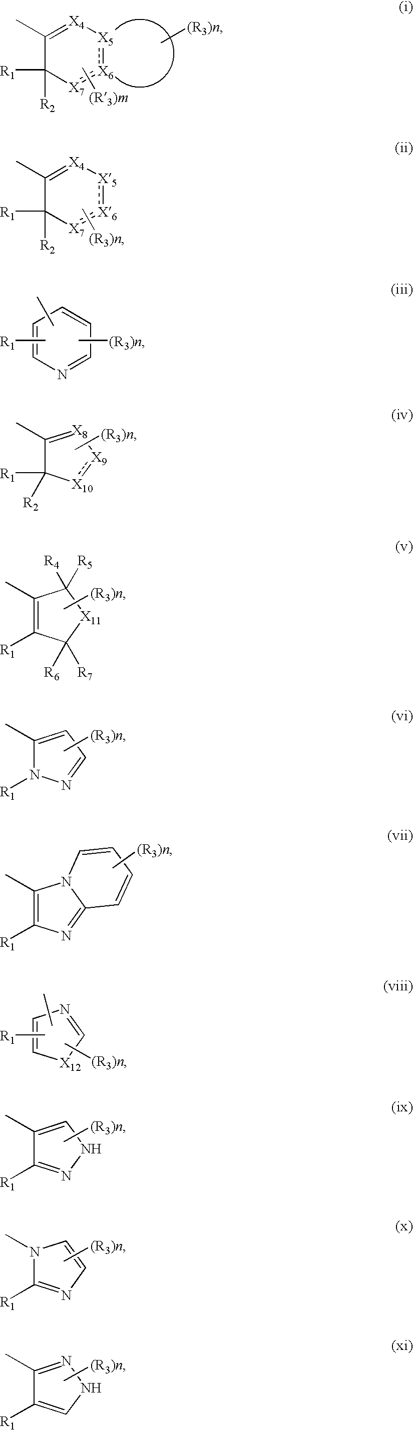

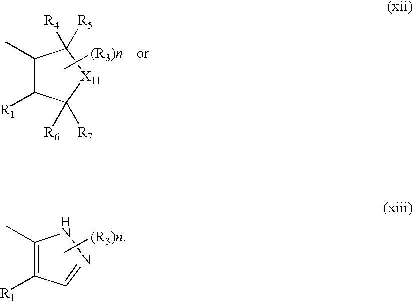

The above-mentioned heterocyclic groups represented by the formulas (i)-(xiii) should contain at least one member selected from N, NH, O, S, SO and SO₂.

In the above-mentioned heterocyclic groups represented by the formulas (i)-(xiii), when the ring constituting member is CH₂, CH or NH, these member are optionally substituted by R₃ or R₃′.

As the “5- to 7-membered ring” of the “5- to 7-membered ring which optionally contains, as a ring-constituting member, one or more members selected from O, N, S, SO and SO₂” represented by the formula:

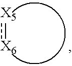

which partially constitutes the fused ring in the heterocyclic group represented by the formula (i):

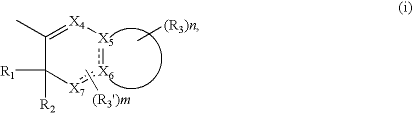

a ring corresponding to a 5- to 7-membered cyclic group included in the aforementioned “cyclic group” of the “optionally substituted cyclic group” for R₁, R₂, R₃, R₃′, R₄, R₅, R₆, R₇, W₁ or W₂ can be mentioned. For example, benzene, a C₅₋₇ cycloalkane, a C₅₋₇ cycloalkene, a C₅₋₇ cycloalkadiene, a 5- to 7-membered aromatic heterocycle [for example, furan, thiophene, pyridine, pyrimidine, pyridazine, pyrazine, pyrrole, imidazole, pyrazole, thiazole, isothiazole, oxazole, isoxazole, oxadiazole, thiadiazole, triazole, tetrazole, triazine etc.], a 5- to 7-membered non-aromatic heterocycle [for example, pyrrolidine, piperidine, morpholine, thiomorpholine, piperazine, hexamethylenimine, oxazolidine, thiazolidine, imidazolidine, oxazoline, thiazoline, imidazoline, dioxole, dioxolane, dihydrooxadiazole, pyran, tetrahydropyran, thiopyran, tetrahydrothiopyran, tetrahydrofuran, pyrazolidine, pyrazoline, tetrahydropyrimidine, dihydrotriazole, tetrahydrotriazole etc.] and the like can be mentioned.

As the “3- to 7-membered ring” of the “3- to 7-membered ring which optionally contains, as a ring-constituting member, one or more members selected from O, N, S, SO and SO₂”, formed by two R₃ together with two adjacent atoms they are bonded to, a ring corresponding to a 3- to 7-membered cyclic group included in the aforementioned “cyclic group” of the “optionally substituted cyclic group” for R₁, R₂, R₃, R₃′, R₄, R₅, R₆, R₇, W₁ or W₂ can be mentioned. For example, benzene, a C₃₋₇ cycloalkane, a C₃₋₇ cycloalkene, a C₄₋₇ cycloalkadiene, a 3- to 7-membered aromatic heterocycle [for example, furan, thiophene, pyridine, pyrimidine, pyridazine, pyrazine, pyrrole, imidazole, pyrazole, thiazole, isothiazole, oxazole, isoxazole, oxadiazole, thiadiazole, triazole, tetrazole, triazine etc.], a 3- to 7-membered non-aromatic heterocycle [for example, aziridine, azetidine, pyrrolidine, piperidine, morpholine, thiomorpholine, piperazine, hexamethylenimine, oxazolidine, thiazolidine, imidazolidine, oxazoline, thiazoline, imidazoline, dioxole, dioxolane, dihydrooxadiazole, pyran, tetrahydropyran, thiopyran, tetrahydrothiopyran, tetrahydrofuran, pyrazolidine, pyrazoline, tetrahydropyrimidine, dihydrotriazole, tetrahydrotriazole etc.] and the like can be mentioned.

R₄ and R₅ in combination optionally form an oxo group, and R₆ and R₇ in combination optionally form an oxo group, provided that at least one of a pair of R₄ and R₅ and a pair of R₆ and R₇ should form an oxo group.

[symbol] is a single bond or a double bond,

provided that
when

### —X₁X₂— is —X₁═X₂—,

then
—X₂ [symbol] X₃— should be —X₂—X₃—,
when

### X₅X₆ is X₅═X₆,

then
X₆ [symbol] X₇ should be X₆—X₇, and
when

### X₅′X₆′ is X₅′═X₆′,

then
X₆′ [symbol] X₇ should be X₆′—X₇.

In compound (Ia),

1) when the group represented by the formula:

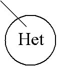

is a heterocyclic group represented by the formula:

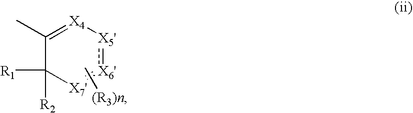

then at least one of R₁ and R₂ should be an optionally substituted aryl group or an optionally substituted heteroaryl group;
2) when the group represented by the formula:

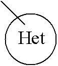

is a heterocyclic group represented by the formula:

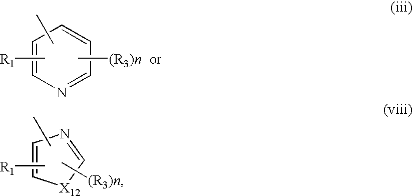

then the carbon atom to which the group represented by the formula:

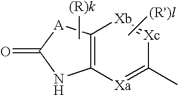

is bonded and the carbon atom to which R₁ is bonded should be adjacent to each other, and R₁ should be an optionally substituted aryl group or an optionally substituted heteroaryl group;
3) when the group represented by the formula:
—X₁ [symbol] X₂ [symbol] X₃— is —CH₂—O—, and the group represented by the formula:

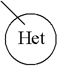

is a heterocyclic group represented by the formula:

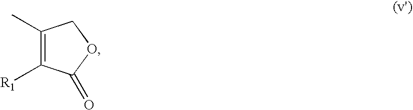

then R₁ should not be phenyl, 4-methoxyphenyl, 3,4-dimethoxyphenyl and 4-chlorophenyl;
4) when the group represented by the formula:
—X₁ [symbol] X₂ [symbol] X₃— is —CH₂—O—, and the group represented by the formula:

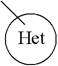

is a heterocyclic group represented by the formula:

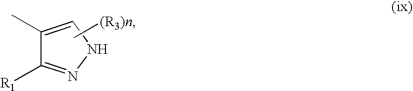

then R₁ should not be an optionally substituted 2-pyridyl;
5) when the group represented by the formula:
—X₁ [symbol] X₂ [symbol] X₃— is —CH₂—O—, and the group represented by the formula:

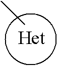

is a heterocyclic group represented by the formula:

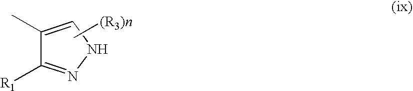

wherein R₁ is an optionally substituted phenyl, then —NH— group in the pyrazole ring as illustrated above should be substituted by R₃,

[provided that in

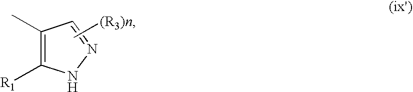

—NH— group in the pyrazole ring as illustrated above may or may not be substituted by R₃];
6) when the group represented by the formula:
—X₁ [symbol] X₂ [symbol] X₃— is —O—, —CH₂—O—, —CH₂—S— or —CH═CH—, and the group represented by the formula:

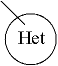

is a heterocyclic group represented by the formula:

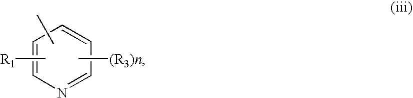

then R₁ should not be a halogen atom and trifluoromethyl;
7) when the group represented by the formula:
—X₁ [symbol] X₂ [symbol] X₃— is —NH— or —CH₂—NH—, and the group represented by the formula:

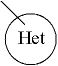

is a heterocyclic group represented by the formula:

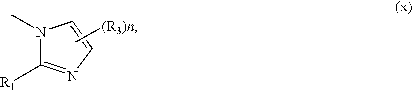

then R₁ should not be an alkyl group;
8) when the group represented by the formula:
—X₁ [symbol] X₂ [symbol] X₃— is —CH₂—O—, and the group represented by the formula:

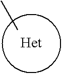

is a heterocyclic group represented by the formula:

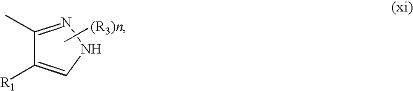

then R₁ should be an optionally substituted aryl group or an optionally substituted heteroaryl group;
9) when the group represented by the formula:
—X₁ [symbol] X₂ [symbol] X₃— is —S— or —CH₂—O—, and the group represented by the formula:

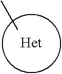

is a heterocyclic group represented by the formula:

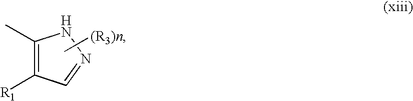

then R₁ should not be a halogen atom; and
10) when the group represented by the formula:

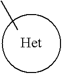

is a heterocyclic group represented by the formula:

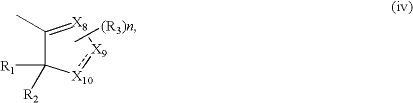

then at least one of R₁ and R₂ should be an optionally substituted aryl group or an optionally substituted heteroaryl group.

In compound (I),

1) when the group represented by the formula:

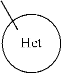

is a heterocyclic group represented by the formula:

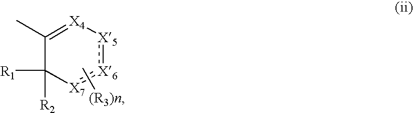

then at least one of R₁ and R₂ should be an optionally substituted aryl group or an optionally substituted heteroaryl group;
2) when the group represented by the formula:

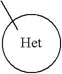

is a heterocyclic group represented by the formula:

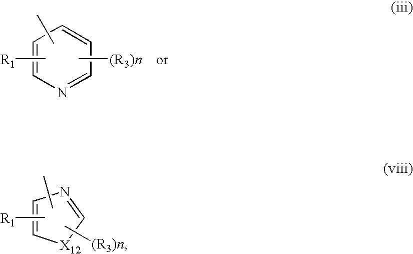

then the carbon atom to which the group represented by the formula:

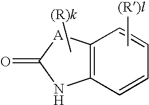

is bonded and the carbon atom to which R₁ is bonded should be adjacent to each other, and R₁ should be an optionally substituted aryl group or an optionally substituted heteroaryl group;
3) when the group represented by the formula:
—X₁ [symbol] X₂ [symbol] X₃— is —CH₂—O—, and the group represented by the formula:

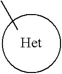

is a heterocyclic group represented by the formula:

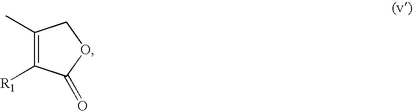

then R₁ should not be phenyl, 47-methoxyphenyl, 3,4-dimethoxyphenyl and 4-chlorophenyl;
4) when the group represented by the formula:
—X₁ [symbol] X₂ [symbol] X₃— is —CH₂—O—, and the group represented by the formula:

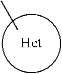

is a heterocyclic group represented by the formula:

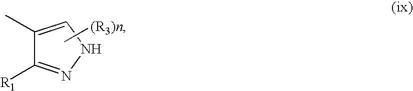

then R₁ should not be an optionally substituted 2-pyridyl;
5) when the group represented by the formula:
—X₁ [symbol] X₂ [symbol] X₃— is —CH₂—O—, and the group represented by the formula:

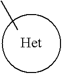

is a heterocyclic group represented by the formula:

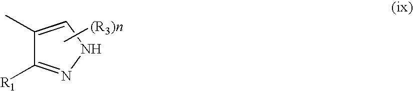

wherein R₁ is an optionally substituted phenyl, then —NH— group in the pyrazole ring as illustrated above should be substituted by R₃,

[provided that in

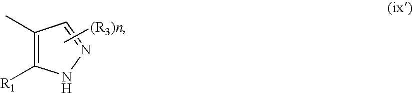

—NH— group in the pyrazole ring as illustrated above may or may not be substituted by R₃];
6) when the group represented by the formula:
—X₁ [symbol] X₂ [symbol] X₃— is —O— —CH₂—O—, —CH₂—S— or —CH═CH—, and the group represented by the formula:

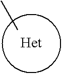

is a heterocyclic group represented by the formula:

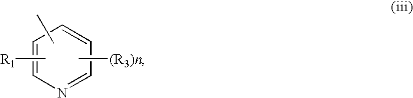

then R₁ should not be a halogen atom and trifluoromethyl;
7) when the group represented by the formula:
—X₁ [symbol] X₂ [symbol] X₃— is —NH— or —CH₂—NH—, and the group represented by the formula:

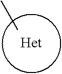

is a heterocyclic group represented by the formula:

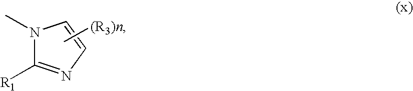

then R₁ should not be an alkyl group; and
8) when the group represented by the formula:

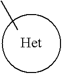

is a heterocyclic group represented by the formula:

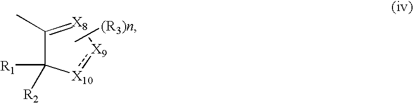

then at least one of R₁ and R₂ should be an optionally substituted aryl group or an optionally substituted heteroaryl group.

In compounds (Ia′) and (I′),

1) at least one of W₁ and W₂ should be an optionally substituted cyclic group; and
2) when W₂ is a hydrogen atom, then W₁ should not be an optionally substituted phenyl.

In compound (Ia′), moreover,

3) at least one of Xₐ, X_{b} and X_{c}′ should be N.

Preferable examples of each group are as follows.

In A, preferably, X₁ is a chemical bond or CH₂ (particularly a chemical bond), X₂ is a chemical bond, CH₂, CH, O, NH, N, S, SO or SO₂, and X₃ is CH₂, CH, O, NH, N, S, SO or SO₂.

More preferably, X₁ is a chemical bond or CH₂ (particularly a chemical bond), X₂ is a chemical bond, CH₂, CH or O, and X₃ is CH₂, CH, O, NH or S.

Furthermore preferably, X₁ is a chemical bond, X₂ is a chemical bond or CH₂, and X₃ is CH₂, O or S.

Still more preferably, X₁ is a chemical bond, X₂ is a chemical bond or CH₂, and X₃ is O or S.

Particularly preferably, X₁ is a chemical bond, X₂ is CH₂, and X₃ is O.

None, one or two of X₁, X₂ and X₃ is preferably a hetero atom, and more preferably, none or one of X₁, X₂ and X₃ is a hetero atom.

—X₁ [symbol] X₂ [symbol] X₃— is preferably —CH₂—O—, —NH—, —CH₂—CH₂—O—, —CH═CH—, —O—CH₂—, —CH₂—S—, —O— or —CH₂—, more preferably —CH₂—O—, —CH₂—S—, —O— or —CH₂—, furthermore preferably —CH₂—O—, —CH₂—S— or —O—, particularly preferably —CH₂—O—.

A group represented by the formula:

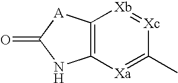

is preferably a group represented by the formula:

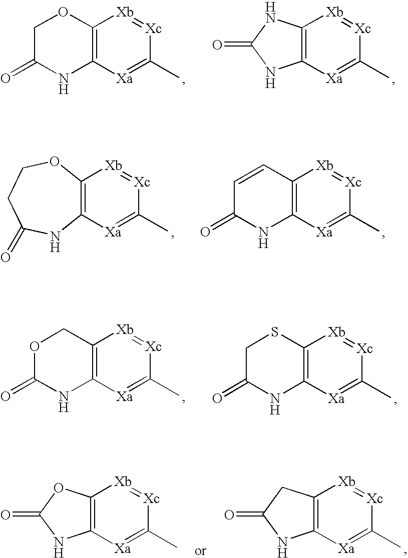

more preferably a group represented by the formula:

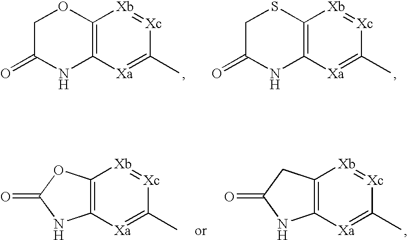

furthermore preferably a group represented by the formula:

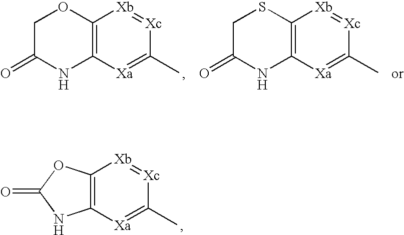

particularly preferably a group represented by the formula:

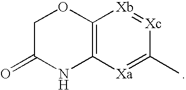

R is preferably an optionally substituted aliphatic hydrocarbon group, an optionally substituted hydroxy group, a halogen atom or a cyano group, or two R optionally form a spiro ring together with a carbon atom they are bonded to.

k is preferably an integer of 0 to 2, more preferably 0.

R′ is preferably an optionally substituted aliphatic hydrocarbon group, an optionally substituted hydroxy group, a halogen atom, a cyano group, an optionally substituted amino group or a nitro group, more preferably an optionally substituted C₁₋₆ alkyl group, a C₁₋₆ alkoxy group, a halogen atom, an amino group or a nitro group, furthermore preferably,

(1) a halogen atom (preferably fluorine atom, chlorine atom, bromine atom);
(2) a C₁₋₆ alkyl group (preferably methyl) optionally substituted by 1 to 3 hydroxy groups;
(3) a C₁₋₆ alkoxy group (preferably methoxy);
(4) an amino group; or
(5) a nitro group,
particularly preferably,
(1) a halogen atom (preferably fluorine atom, chlorine atom); or
(2) a C₁₋₆ alkyl group (preferably methyl).

l is preferably an integer of 0 to 2, more preferably 0 or 1, particularly preferably 0.

Xₐ is preferably CH.
X_{b} is preferably CH.
X_{c} is preferably CH.

Preferable embodiment of the group represented by formula:

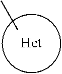

is a heterocyclic group represented by formula:

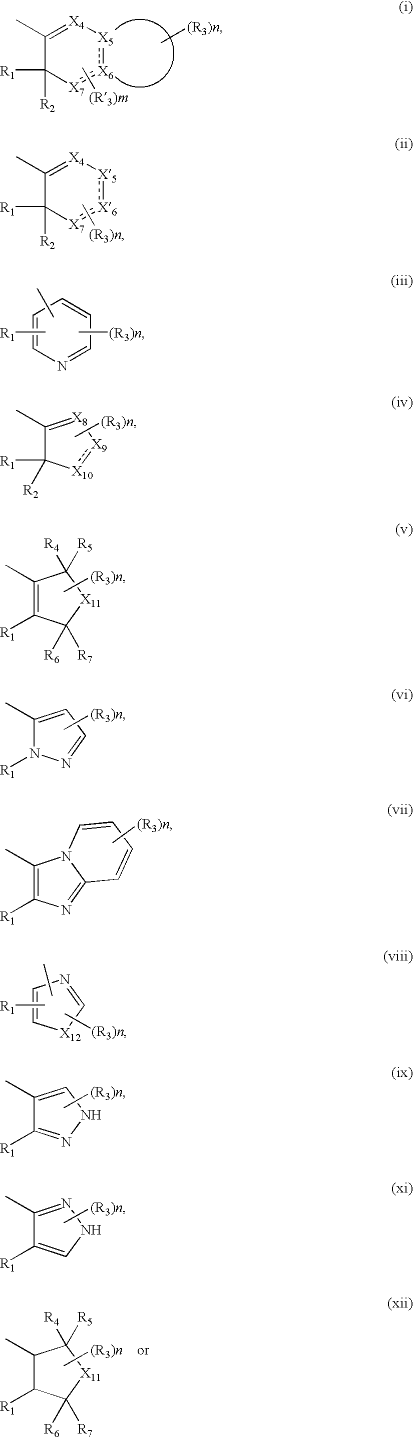

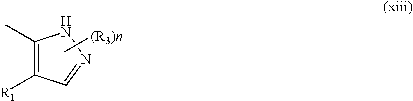

wherein each symbol is as defined above.

Another preferable embodiment is a heterocyclic group represented by formula:

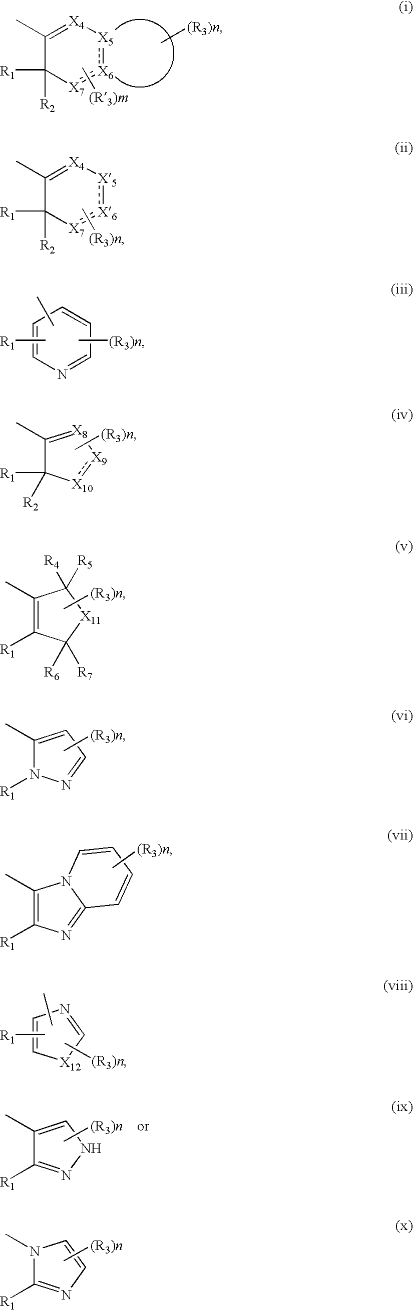

wherein each symbol is as defined above.

Another preferable embodiment is a heterocyclic group represented by formula:

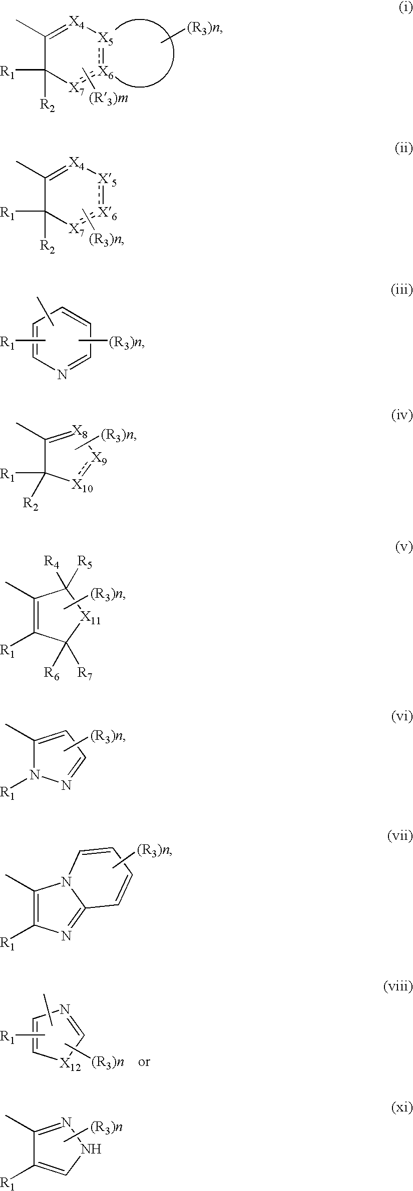

wherein each symbol is as defined above.

More preferable embodiment is a heterocyclic group represented by formula:

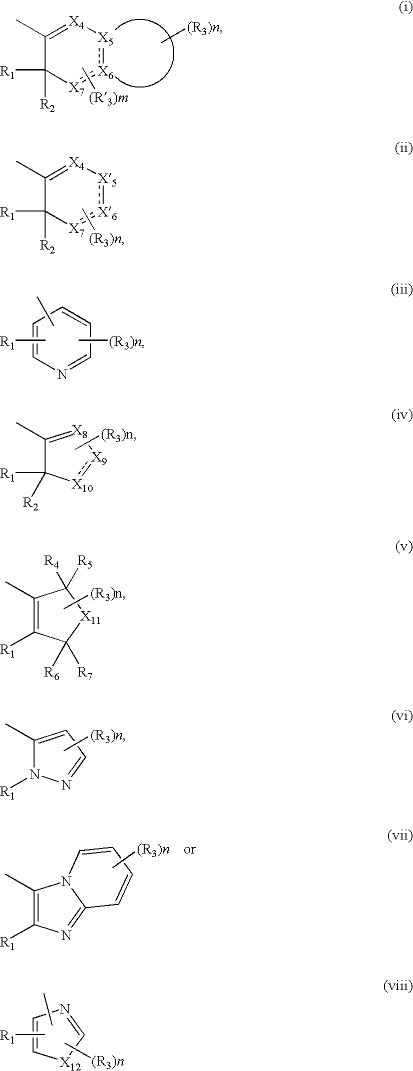

wherein each symbol is as defined above.

Particularly preferable embodiment is a heterocyclic group represented by formula:

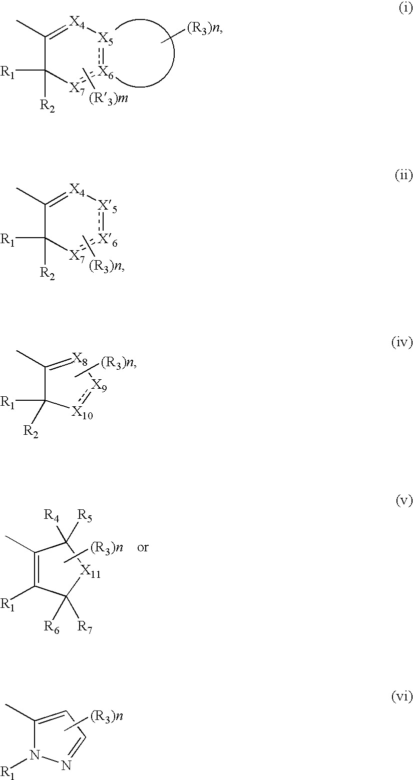

wherein each symbol is as defined above.

Another particularly preferable embodiment is a heterocyclic group represented by formula:

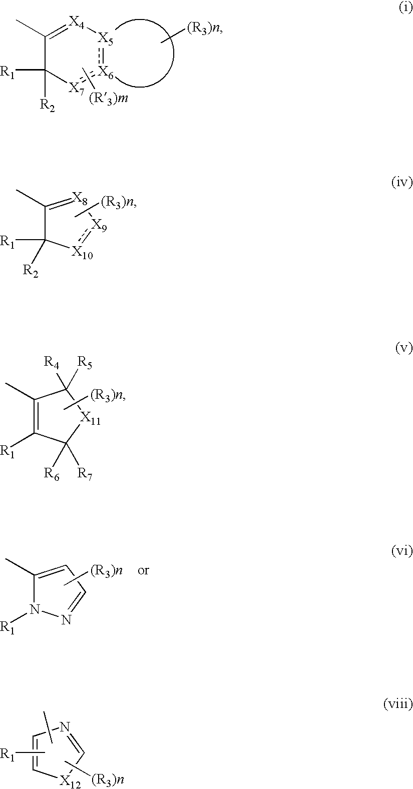

wherein each symbol is as defined above.

Each symbol in the above-mentioned formulas (i)-(xiii) is preferably as follows.

The “5- to 7-membered ring which optionally contains, as a ring-constituting member, one or more members selected from O, N, S, SO and SO₂” represented by the formula:

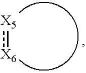

which partially constitutes the fused ring in the heterocyclic group represented by the formula (i):

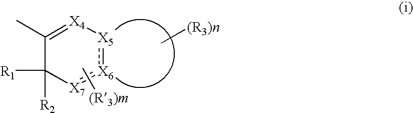

is preferably a 5- to 7-membered ring which optionally contains, as a ring-constituting member, 1 to 3 members selected from O, N, S, SO and SO₂, more preferably a 5- or 6-membered ring which optionally contains, as a ring-constituting member, 1 to 3 members selected from O, N, S, SO and SO₂.

As preferable specific examples of the heterocyclic group represented by the formula:

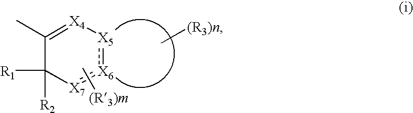

heterocyclic groups represented by the formulas:

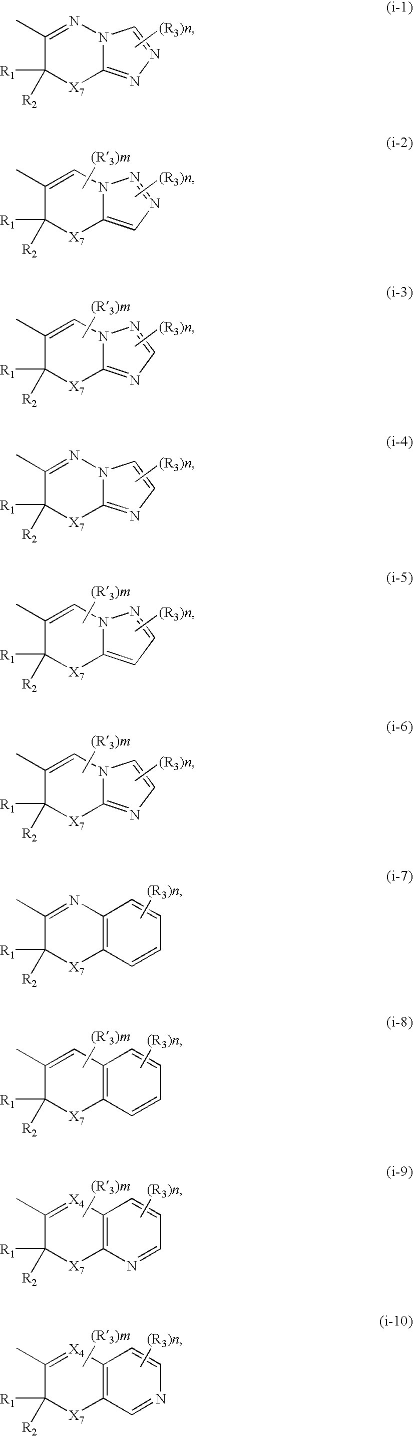

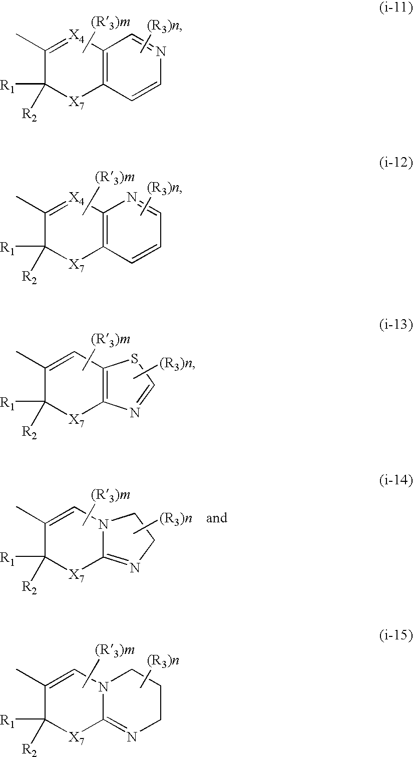

wherein each symbol is as defined above,
can be mentioned. Of these, heterocyclic groups represented by the formulas:

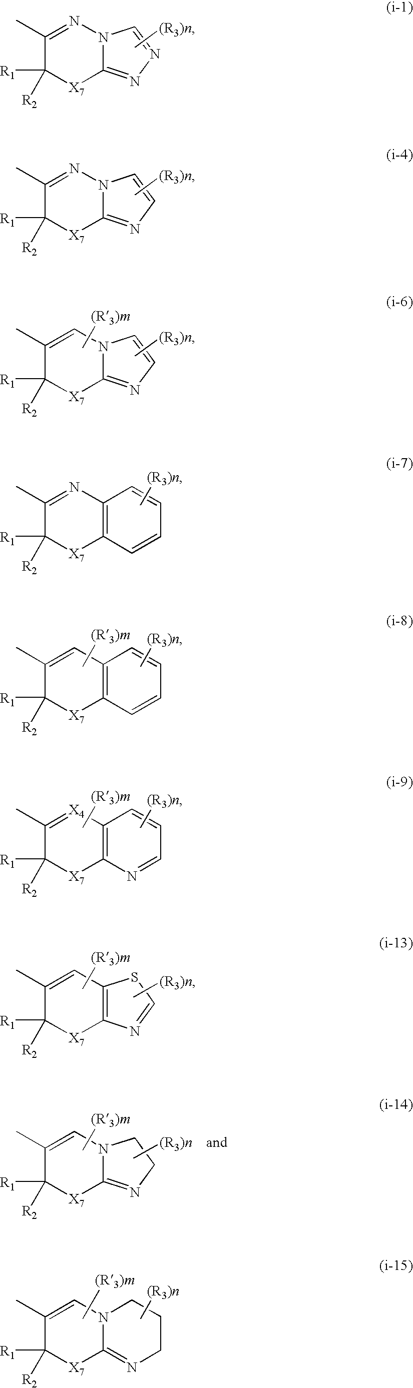

wherein each symbol is as defined above, are preferable.

Other preferable specific examples are heterocyclic groups represented by the formulas:

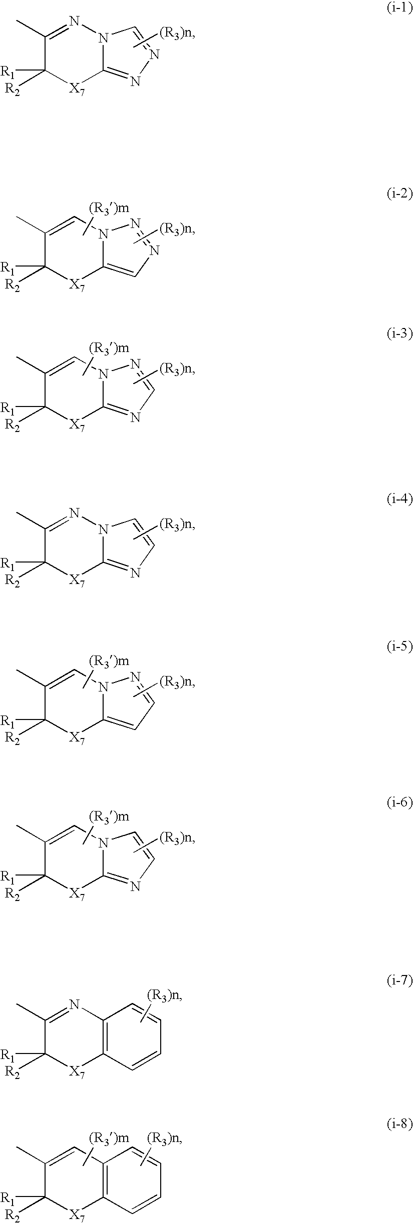

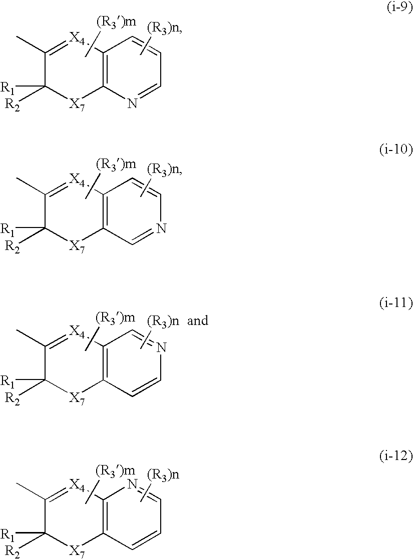

wherein each symbol is as defined above, can be mentioned. Of these, heterocyclic groups represented by the formulas:

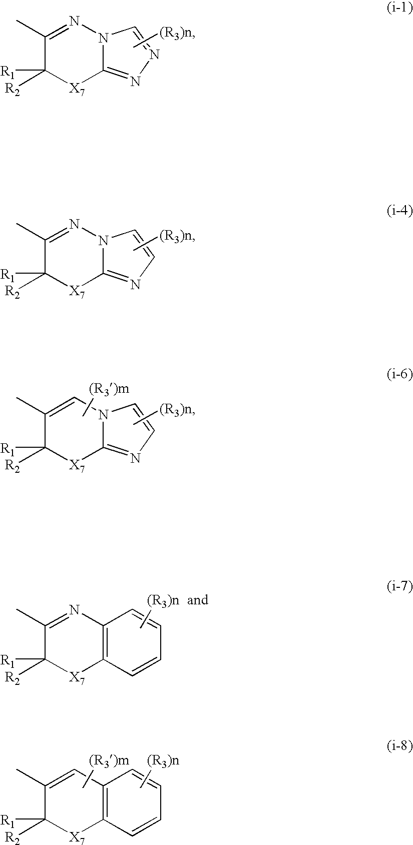

wherein each symbol is as defined above, are preferable.

Particularly, heterocyclic groups represented by the formulas:

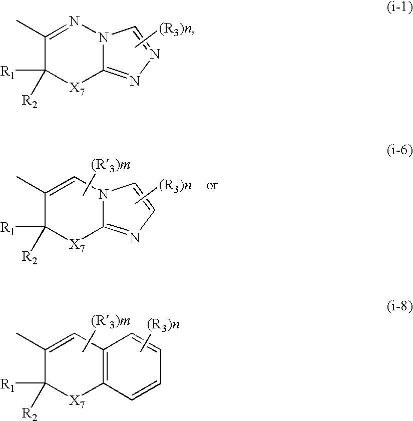

wherein each symbol is as defined above, are preferable.

R₁ and R₂ are the same or different and each is preferably a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, more preferably a hydrogen atom, an optionally substituted aliphatic chain hydrocarbon group, an optionally substituted hydroxy group or an optionally substituted cyclic group, furthermore preferably a hydrogen atom, an optionally substituted C₁₋₆ alkyl group, an optionally substituted C₁₋₆ alkoxy group, an optionally substituted C₆₋₁₄ aryl group or an optionally substituted aromatic heterocyclic group, still more preferably,

(1) a hydrogen atom;
(2) an C₁₋₆ alkyl group (preferably methyl, ethyl, propyl) optionally substituted by 1 to 3 C₆₋₁₂ aryl groups (preferably phenyl) optionally substituted by 1 to 3 C₁₋₆ alkyl groups (preferably methyl);
(3) a C₁₋₆ alkoxy group (preferably methoxy);
(4) a C₆₋₁₄ aryl group (preferably phenyl, naphthyl) optionally substituted by 1 to 3 substituents selected from

(a) a halogen atom (preferably fluorine atom, chlorine atom, bromine atom),

(b) a C₁₋₆ alkyl group (preferably methyl, ethyl, propyl, isopropyl) optionally substituted by 1 to 3 halogen atoms (preferably fluorine atom),

(c) a C₁₋₆ alkoxy group (preferably methoxy) optionally substituted by 1 to 3 halogen atoms (preferably fluorine atom),

(d) a hydroxy group,

(e) a cyano group,

(f) an amino group, and

(g) a nitro group; or

(5) an aromatic heterocyclic group (preferably pyridyl, thiazolyl, thienyl) optionally substituted by 1 to 3 C₁₋₆ alkoxy-carbonyl groups (preferably methoxycarbonyl), particularly preferably,
(1) a hydrogen atom;
(2) a C₁₋₆ alkyl group (preferably methyl, ethyl, propyl) optionally substituted by 1 to 3 C₆₋₁₂ aryl groups (preferably phenyl);
(3) a C₁₋₆ alkoxy group (preferably methoxy);
(4) a C₆₋₁₄ aryl group (preferably phenyl, naphthyl) optionally substituted by 1 to 3 substituents selected from

(a) a halogen atom (preferably fluorine atom, chlorine atom),

(b) a C₁₋₆ alkyl group (preferably methyl, ethyl, propyl, isopropyl) optionally substituted by 1 to 3 halogen atoms (preferably fluorine atom),

(c) a C₁₋₆ alkoxy group (preferably methoxy) optionally substituted by 1 to 3 halogen atoms (preferably fluorine atom), and

(d) a nitro group; or

(5) an aromatic heterocyclic group (preferably pyridyl).

When one of R₁ and R₂ is a hydrogen atom, then the other is preferably other than a hydrogen atom,

R₃ and R₃′ are the same or different and each is preferably an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group, more preferably an optionally substituted aliphatic chain hydrocarbon group, an optionally substituted hydroxy group, an optionally substituted amino group, an optionally esterified carboxyl group, an optionally substituted carbamoyl group, a halogen atom, a cyano group, an optionally substituted imino group, an oxo group, an acyl group or an optionally substituted cyclic group, furthermore preferably an optionally substituted C₁₋₆ alkyl group, an optionally substituted C₂₋₆ alkenyl group, an optionally substituted C₁₋₆ alkoxy group, an optionally substituted C₆₋₁₄ aryl group, an optionally substituted C₁₋₆ alkyl-carbonyl group, an optionally substituted C₁₋₆ alkoxy-carbonyl group, an optionally substituted C₆₋₁₄ aryl-carbonyl group, an optionally substituted C₁₋₆ alkylsulfonyl group, an optionally substituted aromatic heterocyclic group, a halogen atom, a carboxyl group, a cyano group, an optionally substituted amino group, an optionally substituted carbamoyl group, an optionally substituted imino group or an oxo group, still more preferably,

(1) a C₁₋₆ alkyl group (preferably methyl, ethyl, propyl, isopropyl, butyl) optionally substituted by 1 to 5 substituents selected from

(a) a halogen atom (preferably fluorine atom),

(b) a hydroxy group,

(c) a C₁₋₆ alkoxy group (preferably ethoxy),

(d) a C₁₋₆ alkoxy-carbonyl group (preferably methoxycarbonyl),

(e) a C₁₋₆ alkyl-carbonyloxy group (preferably acetyloxy), and

(f) a C₆₋₁₂ aryl group (preferably phenyl);

(2) a C₂₋₆ alkenyl group (preferably vinyl) optionally substituted by 1 to 3 C₁₋₆ alkoxy-carbonyl groups (preferably methoxycarbonyl);
(3) a C₁₋₆ alkoxy group (preferably methoxy, ethoxy) optionally substituted by 1 to 3 hydroxy groups;
(4) a C₆₋₁₄ aryl group (preferably phenyl) optionally substituted by 1 to 3 halogen atoms (preferably fluorine atom, chlorine atom);
(5) a C₁₋₆ alkyl-carbonyl group (preferably acetyl) optionally substituted by 1 to 3 halogen atoms (preferably fluorine atom);
(6) a C₁₋₆ alkoxy-carbonyl group (preferably methoxycarbonyl, ethoxycarbonyl);
(7) a C₆₋₁₄ aryl-carbonyl group (preferably benzoyl);
(8) a C₁₋₆ alkylsulfonyl group (preferably methylsulfonyl);
(9) an aromatic heterocyclic group (preferably pyridyl);
(10) a halogen atom (preferably fluorine atom, chlorine atom, bromine atom, iodine atom);
(11) a hydroxy group;
(12) a carboxyl group;
(13) a cyano group;
(14) an amino group optionally substituted by 1 or 2 C₁₋₁₀ alkyl groups (preferably methyl, ethyl, propyl, tert-butyl, heptyl) optionally substituted by 1 to 3 substituents selected from

(a) a hydroxy group,

(b) a C₁₋₆ alkoxy group (preferably methoxy),

(c) an amino group optionally substituted by 1 or 2 C₁₋₆ alkyl groups (preferably methyl), and

(d) a C₆₋₁₂ aryloxy group (preferably phenoxy);

(15) a carbamoyl group optionally substituted by 1 or 2 C₁₋₆ alkyl groups (preferably methyl);
(16) an imino group; or
(17) an oxo group,
particularly preferably,
(1) a C₁₋₆ alkyl group (preferably methyl, ethyl, propyl, butyl) optionally substituted by 1 to 5 substituents selected from

(a) a halogen atom (preferably fluorine atom),

(b) a C₁₋₆ alkoxy group (preferably ethoxy), and

(c) a C₆₋₁₂ aryl group (preferably phenyl);

(2) a C₁₋₆ alkoxy group (preferably methoxy);
(3) a C₆₋₁₄ aryl group (preferably phenyl) optionally substituted by 1 to 3 halogen atoms (preferably fluorine atom, chlorine atom);
(4) a C₁₋₆ alkyl-carbonyl group (preferably acetyl) optionally substituted by 1 to 3 halogen atoms (preferably fluorine atom);
(5) a C₁₋₆ alkoxy-carbonyl group (preferably ethoxycarbonyl);
(6) a C₆₋₁₄ aryl-carbonyl group (preferably benzoyl);
(7) a C₁₋₆ alkylsulfonyl group (preferably methylsulfonyl);
(8) an aromatic heterocyclic group (preferably pyridyl);
(9) a halogen atom (preferably chlorine atom, bromine atom, iodine atom);
(10) a cyano group;
(11) an amino group optionally substituted by 1 or 2 C₁₋₆ alkyl groups (preferably methyl); or
(12) a carbamoyl group optionally substituted by 1 or 2 C₁₋₆ alkyl groups (preferably methyl).

R₄ and R₅ are the same or different and each is preferably a hydrogen atom or an optionally substituted aliphatic chain hydrocarbon group, or R₄ and R₅ in combination optionally form an oxo group, and more preferably R₄ and R₅ are hydrogen atoms, or R₄ and R₅ in combination optionally form an oxo group.

R₆ and R₇ are hydrogen atoms, or R₆ and R₇ in combination optionally form an oxo group.

However, at least one of a pair of R₄ and R₅ and a pair of R₆ and R₇ should form an oxo group;

X₄ is preferably CH or N.

X₅ and X₆ are the same or different and each is preferably CH, C or N, more preferably C or N, and particularly preferably, X₅ is N or C and X₆ is C.

X₅′ and X₆′ are the same or different and each is preferably CH₂, CH, NH or N, more preferably, X₅′ is N, NH or CH and X₆′ is N, CH or CH₂, and particularly, preferably X₅′ is N and X₆′ is CH.

X₇ is preferably CH, O, S, SO or SO₂, more preferably O, S, SO or SO₂.

X₈ is preferably CH or N, more preferably N.

X₉ is preferably CH₂, CH, NH, N or O, more preferably NH.

X₁₀ is preferably CH₂, CH, NH or N, more preferably CH₂.

X₁₁ is preferably NH or O.

X₁₂ is preferably O or S, more preferably S.

X₅ [symbol] X₆ is X₅—X₆ or X₅═X₆.

X₆ [symbol] X₇ is preferably X₆—X₇.

However, when X₅ [symbol] X₆ is X₅═X₆, then X₆ [symbol] X₇ should be X₆—X₇.

X₅ [symbol] X₆′ is X₅′—X₆′ or X₅′═X₆′.

X₆′ [symbol] X₇ is preferably X₆′—X₇.

However, when X₅′ [symbol] X₆′ is X₅′═X₆′, then X₆′ [symbol] X₇ should be X₆′—X₇.

X₉ [symbol] X₁₀ is preferably X₉—X₁₀.

m is preferably 0.

n is preferably an integer of 0 to 2, more preferably 0 or 1.

However, when the group represented by the formula:

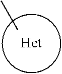

is a heterocyclic group represented by the formula:

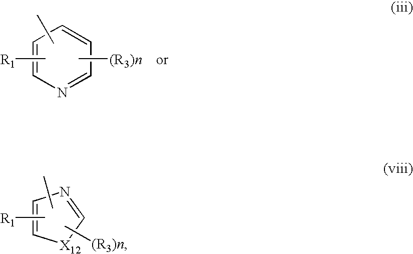

then the carbon atom to which the group represented by the formula:

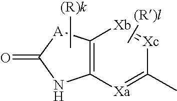

is bonded and the carbon atom to which R₁ is bonded should be adjacent to each other.

At least one of X₄, X₅, X₆ and X₇ is a hetero atom, and preferably, a compound wherein consecutive three or more of X₄, X₅, X₆ and X₇ are hetero atoms is excluded. At least one of X₄, X₅′, X₆′ and X₇ is a hetero atom, and preferably, a compound wherein consecutive three or more of X₄, X₅′, X₆′ and X₇ are hetero atoms is excluded.

W₁ and W₂ are the same or different and each is preferably a hydrogen atom, an optionally substituted cyclic group, an optionally substituted C₁₋₁₀ alkyl group, an optionally substituted hydroxy group or a halogen atom.

More preferably,

W₁ is a hydrogen atom, an optionally substituted C₁₋₁₀ alkyl group, an optionally substituted hydroxy group or a halogen atom; and
W₂ is an optionally substituted heterocyclic group.

Particularly preferably,

W₁ is a hydrogen atom; and
W₂ is an optionally substituted heterocyclic group.

In the present invention, of compounds (Ia′) and (I′), a compound wherein W₁ is a hydrogen atom, and W₂ is an optionally substituted heterocyclic group, i.e., compounds (Ia) and (I), are preferable. Of these compounds (Ia) and (I), the following compound and the like are preferable.

### [Compound A-a]

In compound (Ia), a compound wherein

A is a group represented by the formula:

—X₁ [symbol] X₂ [symbol] X₃—;
wherein
X₁ is a chemical bond or CH₂ (particularly a chemical bond);
X₂ is a chemical bond, CH₂, CH, O, NH, N, S, SO or SO₂;
X₃ is CH₂, CH, O, NH, N, S, SO or SO₂; and
[symbol] is a single bond or a double bond;
provided that
when
X₁ [symbol] X₂— is X₁═X₂—,
then
—X₂ [symbol] X₃— should be —X₂—X₃—;
   R and R′ are the same or different and each is an optionally substituted aliphatic hydrocarbon group, an optionally substituted hydroxy group, an optionally substituted amino group, an optionally esterified carboxyl group, an optionally substituted carbamoyl group, a halogen atom, a nitro group, cyano group, an optionally substituted mercapto group or an acyl group, or two R optionally form a Spiro ring together with a carbon atom they are bonded to;
   k is an integer of 0 to 4;
   l is an integer of 0 to 3;

### Xₐ is CH or N;

### X_{b} is CH or N;

### X_{c} is CH or N; and

a group represented by the formula:

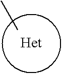

is a heterocyclic group represented by the formula:

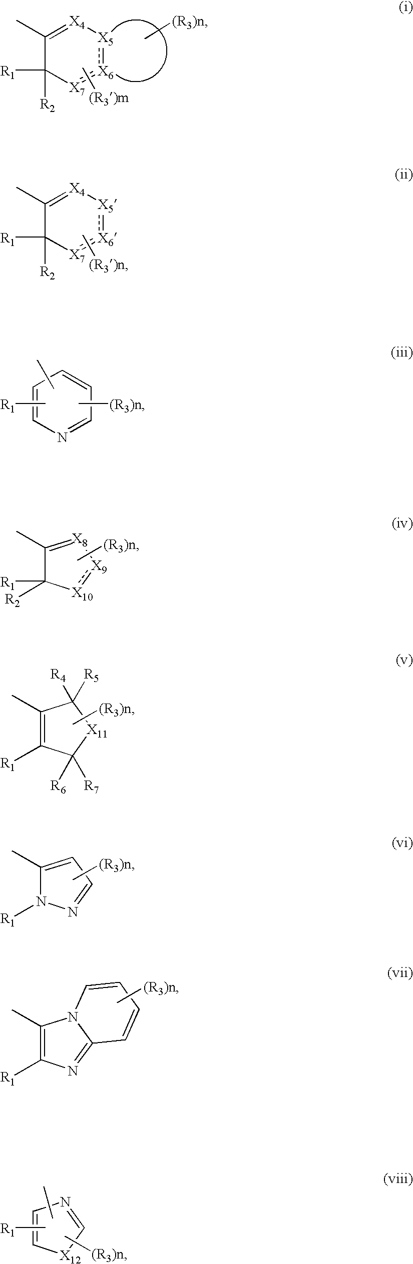

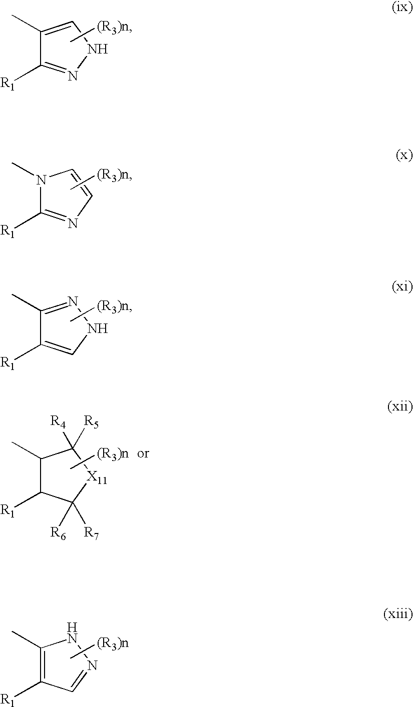

wherein
the formula:

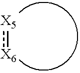

which partially constitutes the fused ring in the heterocyclic group represented by the formula (i):

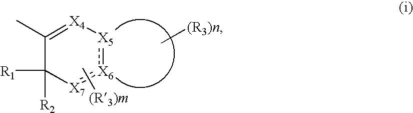

is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group, or
two R₃ optionally form, together with two adjacent atoms they are bonded to, a 3- to 7-membered ring which optionally contains, as a ring-constituting member, one or more members selected from O, N, S, SO and SO₂;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₆ and R₇ in combination optionally form an oxo group;
provided that at least one of a pair of R₄ and R₅ and a pair of R₆ and R₇ should form an oxo group;
X₄ is CH or N;
X₅ and X₆ are the same or different and each is CH, C or N;
X₅′ and X₆′ are the same or different and each is CH₂, CH, NH, N, O, S, SO or SO₂;
X₇ is CH₂, CH, NH, N, O, S, SO or SO₂;
X₈ is CH or N;
X₉ is CH₂, CH, NH, N, O, S, SO or SO₂;
X₁₀ is CH₂, CH, NH, N, O, S, SO or SO₂;
X₁₁ is NH, O, S, SO or SO₂;
X₁₂ is O or S; and
[symbol] is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇; and
m and n are the same or different and each is an integer of 0 to 4.

### [Compound B-a]

In compound (Ia), a compound wherein

A is a group represented by the formula:

—X₁ [symbol] X₂ [symbol] X₃—;
wherein
X₁ is a chemical bond or CH₂ (particularly a chemical bond);
X₂ is a chemical bond, CH₂, CH or O;
X₃ is CH₂, CH, O, NH or S; and
[symbol] is a single bond or a double bond;
provided that
when
—X₁ [symbol] X₂— is —X₁═X₂,
then
—X₂ [symbol] X₃— should be —X₂—X₃—;
   R and R′ are the same or different and each is an optionally substituted aliphatic hydrocarbon group, an optionally substituted hydroxy group, an optionally substituted amino group, a halogen atom, a nitro group or a cyano group, or two R optionally form a spiro ring together with a carbon atom they are bonded to;
   k is an integer of 0 to 4;
   l is an integer of 0 to 3;

### Xₐ is CH or N;

### X_{b} is CH or N;

### X_{c} is CH or N; and

a group represented by the formula:

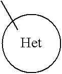

is a heterocyclic group represented by the formula:

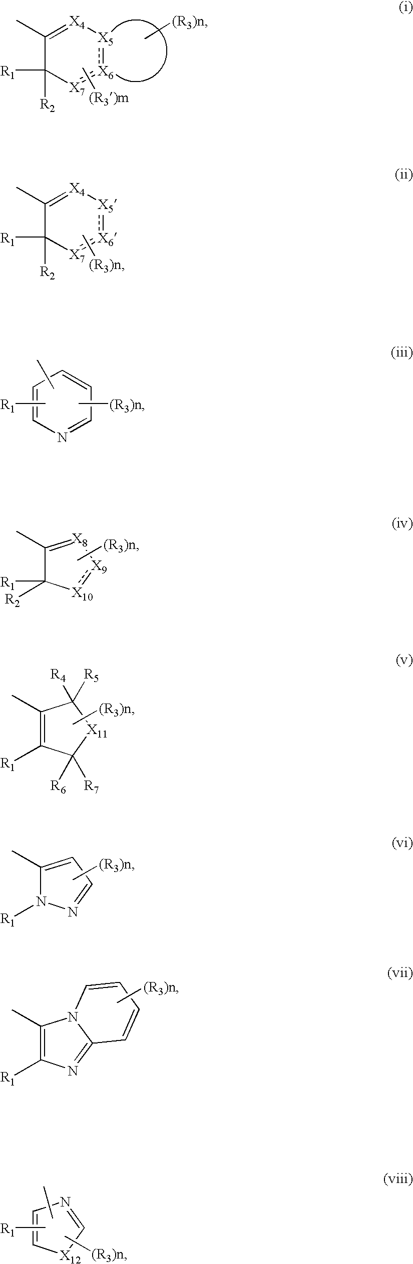

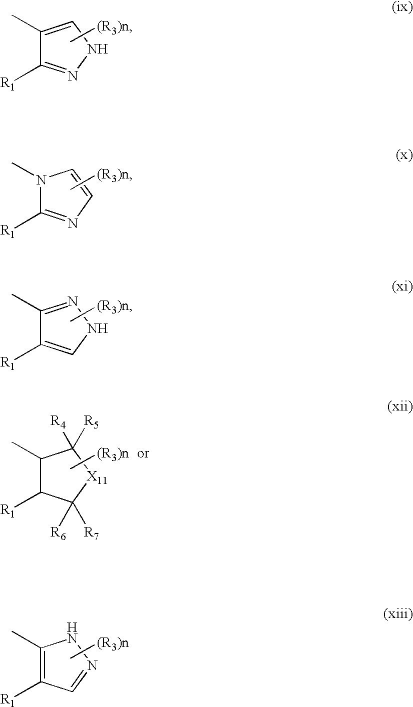

wherein
the formula:

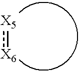

which partially constitutes the fused ring in the heterocyclic group represented by the formula (i):

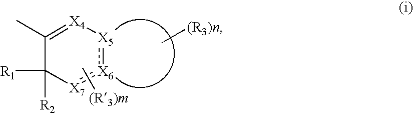

is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an Optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group;
R₄ and R₅ are the same or different and each is a hydrogen atom or an optionally substituted aliphatic chain hydrocarbon group, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group;
X₄ is CH or N;
X₅ and X₆ are the same or different and each is CH, C or N;
X₅′ and X₆′ are the same or different and each is CH₂, CH, NH or N;
X₇ is CH, O, S, SO or SO₂;
X₈ is CH or N;
X₉ is CH₂, CH, NH, N or O;
X₁₀ is CH₂, CH, NH or N;
X₁₁ is NH or O;
X₁₂ is S;
[symbol] is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇; and
m and n are the same or different and each is an integer of 0 to 4.

### [Compound A].

In compound (I), a compound wherein

A is a group represented by the formula:

—X₁ [symbol] X₂ [symbol] X₃—
wherein
X₁ is a chemical bond or CH₂ (particularly a chemical bond);
X₂ is a chemical bond, CH₂, CH, O, NH, N, S, SO or SO₂;
X₃ is CH₂, CH, O, NH, N, S, SO or SO₂; and
[symbol] is a single bond or a double bond;
provided that
when
—X₁ [symbol] X₂— is —X₁═X₂—,
then
—X₂ [symbol] X₃— should be —X₂—X₃—;
   R and R′ are the same or different and each is an optionally substituted aliphatic hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group or an acyl group, or two R optionally form a spiro ring together with a carbon atom they are bonded to;
   k is an integer of 0 to 4;
   l is an integer of 0 to 3; and
   a group represented by the formula:

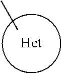

is a heterocyclic group represented by the formula:

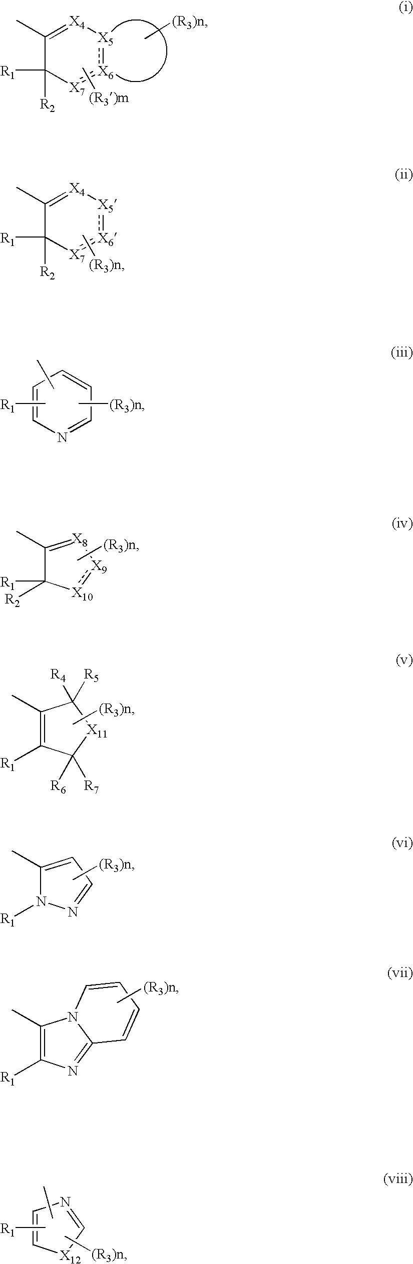

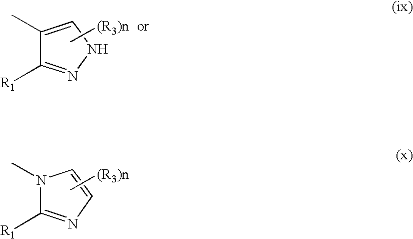

wherein
the formula:

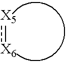

which partially constitutes the fused ring in the heterocyclic group represented by the formula (i):

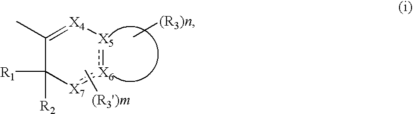

is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group, or
two R₃ optionally form, together with two adjacent atoms they are bonded to, a 3- to 7-membered ring which optionally contains, as a ring-constituting member, one or more members selected from O, N, S, SO and SO₂;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₆ and R₇ in combination optionally form an oxo group;
provided that at least one of a pair of R₄ and R₅ and a pair of R₆ and R₇ should form an oxo group;
X₄ is CH or N;
X₅ and X₆ are the same or different and each is CH, C or N;
X₅′ and X₆′ are the same or different and each is CH₂, CH, NH, N, O, S, SO or SO₂;
X₇ is CH₂, CH, NH, N, O, S, SO or SO₂;
X₈ is CH or N;
X₉ is CH₂, CH, NH, N, O, S, SO or SO₂;
X₁₀ is CH₂, CH, NH, N, O, S, SO or SO₂;
X₁₁ is NH, O, S, SO or SO₂;
X₁₂ is O or S;
[symbol] is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇; and
m and n are the same or different and each is an integer of 0 to 4.

### [Compound B].

In compound (I), a compound wherein

A is a group represented by the formula:

—X₁ [symbol] X₂ [symbol] X₃—
wherein
X₁ is a chemical bond or CH₂ (particularly a chemical bond);
X₂ is a chemical bond, CH₂, CH or O;
X₃ is CH₂, CH, O, NH or S; and
[symbol] is a single bond or a double bond;
provided that
when
—X₁ [symbol] X₂— is —X₁═X₂—,
then
—X₂ [symbol] X₃— should be —X₂—X₃—;
   R and R′ are the same or different and each is an optionally substituted aliphatic hydrocarbon group, an optionally substituted hydroxy group, a halogen atom or a cyano group, or two R optionally form a Spiro ring together with a carbon atom they are bonded to;
   k is an integer of 0 to 4;
   l is an integer of 0 to 3; and
   a group represented by the formula:

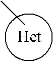

is a heterocyclic group represented by the formula:

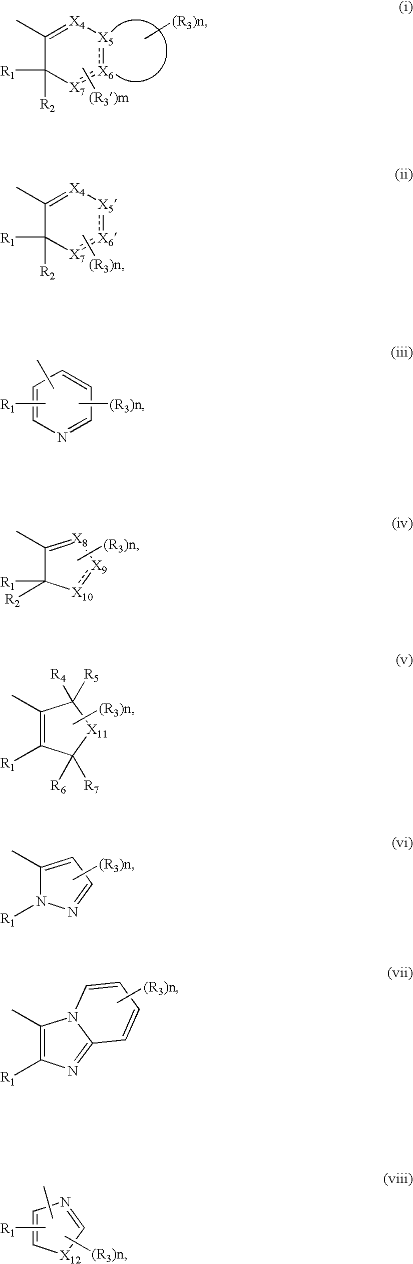

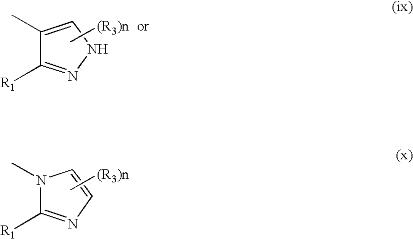

wherein
the formula:

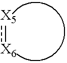

which partially constitutes the fused ring in the heterocyclic group represented by the formula (i):

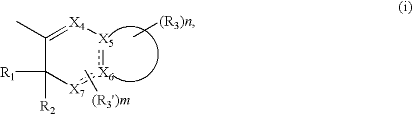

is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;
R₄ and R₅ are the same or different and each is a hydrogen atom or an optionally substituted aliphatic chain hydrocarbon group, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group;
X₄ is CH or N;
X₅ and X₆ are the same or different and each is CH, C or N;
X₅′ and X₆′ are the same or different and each is CH₂, CH, NH or N;
X₇ is O, S, SO or SO₂;
X₈ is CH or N;
X₉ is CH₂, CH, NH, N or O;
X₁₀ is CH₂, CH, NH or N;
X₁₁ is NH or O;
X₁₂ is S;
[symbol] is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇; and
m and n are the same or different and each is an integer of 0 to 4.

### [Compound C-a]

In compound (Ia),

### A is —CH₂—O—, —CH₂—S—, —O— or —CH₂—;

### R′ is

(1) a halogen atom (preferably fluorine atom, chlorine atom, bromine atom);
(2) a C₁₋₆ alkyl group (preferably methyl) optionally substituted by 1 to 3 hydroxy groups;
(3) a C₁₋₆ alkoxy group (preferably methoxy);
(4) an amino group; or
(5) a nitro group;
k is 0;
l is 0 or 1;
Xₐ is CH or N;
X_{b} is CH or N;
X_{c} is CH or N; and
   a group represented by the formula:

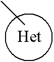

is a heterocyclic group represented by the formula:

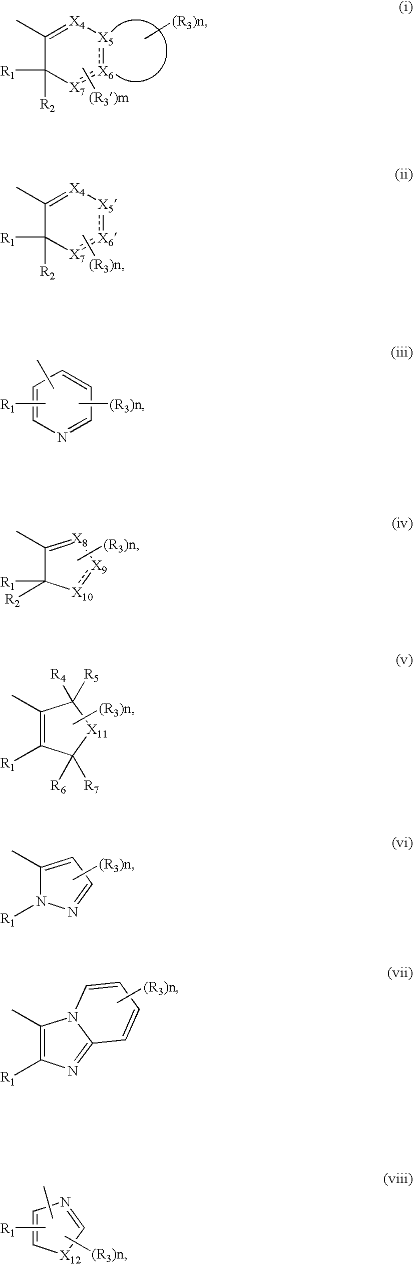

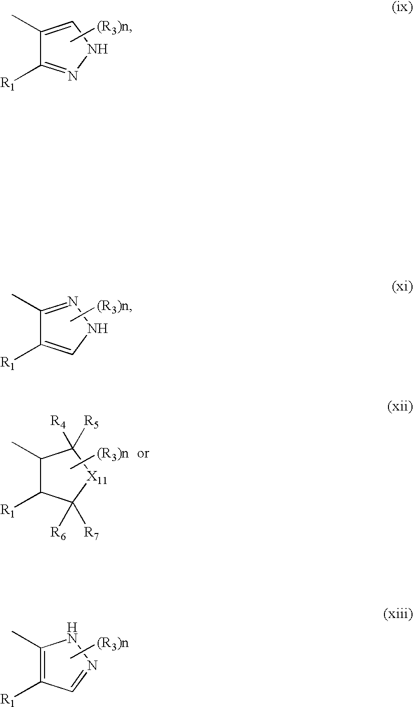

wherein
the formula:

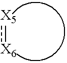

which partially constitutes the fused ring in the heterocyclic group represented by the formula (i):

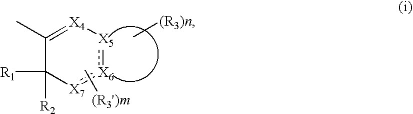

is a 5- to 7-membered ring which optionally contains, as a ring-constituting member, 1 to 3 members selected from O, N, S, SO and SO₂;
R₁ and R₂ are the same or different and each is
(1) a hydrogen atom;
(2) an C₁₋₆ alkyl group (preferably methyl, ethyl, propyl) optionally substituted by 1 to 3 C₆₋₁₂ aryl groups (preferably phenyl) optionally substituted by 1 to 3 C₁₋₆ alkyl groups (preferably methyl);
(3) a C₁₋₆ alkoxy group (preferably methoxy);
(4) a C₆₋₁₄ aryl group (preferably phenyl, naphthyl) optionally substituted by 1 to 3 substituents selected from
   (a) a halogen atom (preferably fluorine atom, chlorine atom, bromine atom),
   (b) a C₁₋₆ alkyl group (preferably methyl, ethyl, propyl, isopropyl) optionally substituted by 1 to 3 halogen atoms (preferably fluorine atom),
   (c) a C₁₋₆ alkoxy group (preferably methoxy) optionally substituted by 1 to 3 halogen atoms (preferably fluorine atom),
   (d) a hydroxy group,
   (e) a cyano group,
   (f) an amino group, and
   (g) a nitro group; or
(5) an aromatic heterocyclic group (preferably pyridyl, thiazolyl, thienyl) optionally substituted by 1 to 3 C₁₋₆ alkoxy-carbonyl groups (preferably methoxycarbonyl);
R₃ is
(1) a C₁₋₆ alkyl group (preferably methyl, ethyl, propyl, isopropyl, butyl) optionally substituted by 1 to 5 substituents selected from
   (a) a halogen atom (preferably fluorine atom),
   (b) a hydroxy group,
   (c) a C₁₋₆ alkoxy group (preferably ethoxy),
   (d) a C₁₋₆ alkoxy-carbonyl group (preferably methoxycarbonyl),
   (e) a C₁₋₆ alkyl-carbonyloxy group (preferably acetyloxy), and
   (f) a C₆₋₁₂ aryl group (preferably phenyl);
(2) a C₂₋₆ alkenyl group (preferably vinyl) optionally substituted by 1 to 3 C₁₋₆ alkoxy-carbonyl groups (preferably methoxycarbonyl);
(3) a C₁₋₆ alkoxy group (preferably methoxy, ethoxy) optionally substituted by 1 to 3 hydroxy groups;
(4) a C₆₋₁₄ aryl group (preferably phenyl) optionally substituted by 1 to 3 halogen atoms (preferably fluorine atom, chlorine atom);
(5) a C₁₋₆ alkyl-carbonyl group (preferably acetyl) optionally substituted by 1 to 3 halogen atoms (preferably fluorine atom);
(6) a C₁₋₆ alkoxy-carbonyl group (preferably methoxycarbonyl, ethoxycarbonyl);
(7) a C₆₋₁₄ aryl-carbonyl group (preferably benzoyl);
(8) a C₁₋₆ alkylsulfonyl group (preferably methylsulfonyl);
(9) an aromatic heterocyclic group (preferably pyridyl);
(10) a halogen atom (preferably fluorine atom, chlorine atom, bromine atom, iodine atom);
(11) a hydroxy group;
(12) a carboxyl group;
(13) a cyano group;
(14) an amino group optionally substituted by 1 or 2 C₁₋₁₀ alkyl groups (preferably methyl, ethyl, propyl, tert-butyl, heptyl) optionally substituted by 1 to 3 substituents selected from
   (a) a hydroxy group,
   (b) a C₁₋₆ alkoxy group (preferably methoxy),
   (c) an amino group optionally substituted by 1 or 2 C₁₋₆ alkyl groups (preferably methyl), and
   (d) a C₆₋₁₂ aryloxy group (preferably phenoxy);
(15) a carbamoyl group optionally substituted by 1 or 2 C₁₋₆ alkyl groups (preferably methyl);
(16) an imino group; or
(17) an oxo group;
R₄ and R₅ are hydrogen atoms, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group;
X₄ is N or CH;
X₅ is N or C;

X₆ is C;
X₅′ is N, NH or CH;
X₆′ is N, CH or CH₂;
X₇ is CH, O, S, SO or SO₂;
X₈ is N;
X₉ is NH;
X₁₀ is CH₂;
X₁₁ is O or NH;
X₁₂ is S;
X₅ [symbol] X₆ is X₅—X₆ or X₅═X₆;
X₆ [symbol] X₇ is X₆—X₇;
X₅′ [symbol] X₆′ is X₅′—X₆′ or X₅′═X₆′;
X₆′ [symbol] X₇ is X₆′—X₇;
X₉ [symbol] X₁₀ is X₉—X₁₀;
m is 0; and
n is 0, 1 or 2.

### [Compound C]

In compound (I), a compound wherein

### A is —CH₂—O—, —CH₂—S—, —O— or —CH₂—;

### R′ is

(1) a halogen atom (preferably fluorine atom, chlorine atom, bromine atom);
(2) a C₁₋₆ alkyl group (preferably methyl) optionally substituted by 1 to 3 hydroxy groups;
(3) a C₁₋₆ alkoxy group (preferably methoxy);
(4) an amino group; or
(5) a nitro group;
k is 0;
l is 0 or 1; and
a group represented by the formula:

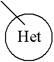

is a heterocyclic group represented by the formula:

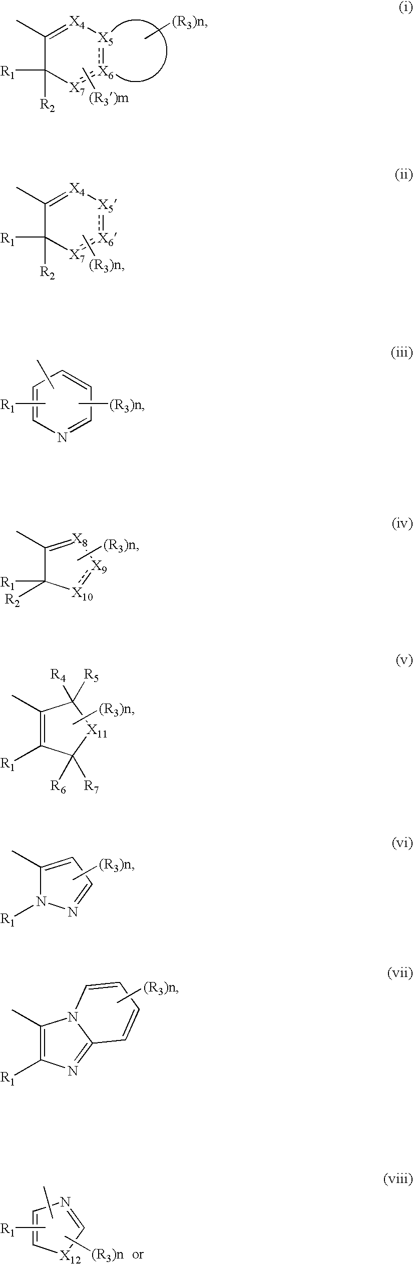

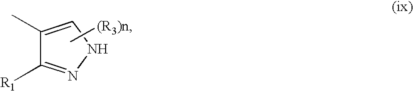

wherein
the formula:

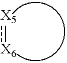

which partially constitutes the fused ring in the heterocyclic group represented by the formula (i):

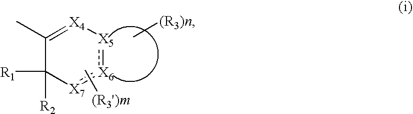

is a 5- to 7-membered ring which optionally contains, as a ring-constituting member, 1 to 3 members selected from O, N, S, SO and SO₂;
R₁ and R₂ are the same or different and each is
(1) a hydrogen atom;
(2) an C₁₋₆ alkyl group (preferably methyl, ethyl, propyl) optionally substituted by 1 to 3 C₆₋₁₂ aryl groups (preferably phenyl) optionally substituted by 1 to 3 C₁₋₆ alkyl groups (preferably methyl);
(3) a C₁₋₆ alkoxy group (preferably methoxy);
(4) a C₆₋₁₄ aryl group (preferably phenyl, naphthyl) optionally substituted by 1 to 3 substituents selected from
   (a) a halogen atom (preferably fluorine atom, chlorine atom, bromine atom),
   (b) a C₁₋₆ alkyl group (preferably methyl, ethyl, propyl, isopropyl) optionally substituted by 1 to 3 halogen atoms (preferably fluorine atom),
   (c) a C₁₋₆ alkoxy group (preferably methoxy) optionally substituted by 1 to 3 halogen atoms (preferably fluorine atom),
   (d) a hydroxy group,
   (e) a cyano group,
   (f) an amino group, and
   (g) a nitro group; or
(5) an aromatic heterocyclic group (preferably pyridyl, thiazolyl, thienyl) optionally substituted by 1 to 3 C₁₋₆ alkoxy-carbonyl groups (preferably methoxycarbonyl);
R₃ is
(1) a C₁₋₆ alkyl group (preferably methyl, ethyl, propyl, isopropyl, butyl) optionally substituted by 1 to 5 substituents selected from
   (a) a halogen atom (preferably fluorine atom),
   (b) a hydroxy group,
   (c) a C₁₋₆ alkoxy group (preferably ethoxy),
   (d) a C₁₋₆ alkoxy-carbonyl group (preferably methoxycarbonyl),
   (e) a C₁₋₆ alkyl-carbonyloxy group (preferably acetyloxy), and
   (f) a C₆₋₁₂ aryl group (preferably phenyl);
(2) a C₂₋₆ alkenyl group (preferably vinyl) optionally substituted by 1 to 3 C₁₋₆ alkoxy-carbonyl groups (preferably methoxycarbonyl);
(3) a C₁₋₆ alkoxy group (preferably methoxy, ethoxy) optionally substituted by 1 to 3 hydroxy groups;
(4) a C₆₋₁₄ aryl group (preferably phenyl) optionally substituted by 1 to 3 halogen atoms (preferably fluorine atom, chlorine atom);
(5) a C₁₋₆ alkyl-carbonyl group (preferably acetyl) optionally substituted by 1 to 3 halogen atoms (preferably fluorine atom);
(6) a C₁₋₆ alkoxy-carbonyl group (preferably methoxycarbonyl, ethoxycarbonyl);
(7) a C₆₋₁₄ aryl-carbonyl group (preferably benzoyl);
(8) a C₁₋₆ alkylsulfonyl group (preferably methylsulfonyl);
(9) an aromatic heterocyclic group (preferably pyridyl);
(10) a halogen atom (preferably fluorine atom, chlorine atom, bromine atom, iodine atom);
(11) a hydroxy group;
(12) a carboxyl group;
(13) a cyano group;
(14) an amino group optionally substituted by 1 or 2 C₁₋₁₀ alkyl groups (preferably methyl, ethyl, propyl, tert-butyl, heptyl) optionally substituted by 1 to 3 substituents selected from
   (a) a hydroxy group,
   (b) a C₁₋₆ alkoxy group (preferably methoxy),
   (c) an amino group optionally substituted by 1 or 2 C₁₋₆ alkyl groups (preferably methyl), and
   (d) a C₆₋₁₂ aryloxy group (preferably phenoxy);
(15) a carbamoyl group optionally substituted by 1 or 2 C₁₋₆ alkyl groups (preferably methyl); or
(16) an oxo group;
R₄ and R₅ are hydrogen atoms, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group;
X₄ is N or CH;
X₅ is N or C;

X₆ is C;
X₅′ is N, NH or CH;
X₆′ is N, CH or CH₂;
X₇ is CH, O, S, SO or SO₂;
X₈ is N;
X₉ is NH;
X₁₀ is CH₂;
X₁₁ is O or NH;
X₁₂ is S;
X₅ [symbol] X₆ is X₅—X₆ or X₅═X₆;
X₆ [symbol] X₇ is X₆—X₇;
X₅′ [symbol] X₆′ is X₅′—X₆′ or X₅′═X₆′;
X₆′ [symbol] X₇ is X₆′—X₇;
X₉ [symbol] X₁₀ is X₉—X₁₀;
m is 0; and
n is 0, 1 or 2.

### [Compound D]

In compound (I), a compound wherein

### A is —CH₂—O—, —O— or —CH₂—S—;

### R′ is

(1) a halogen atom (preferably fluorine atom, chlorine atom); or
(2) a C₁₋₆ alkyl group (preferably methyl);
k is 0;
l is 0 or 1; and
a group represented by the formula:

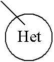

is a heterocyclic group represented by the formula:

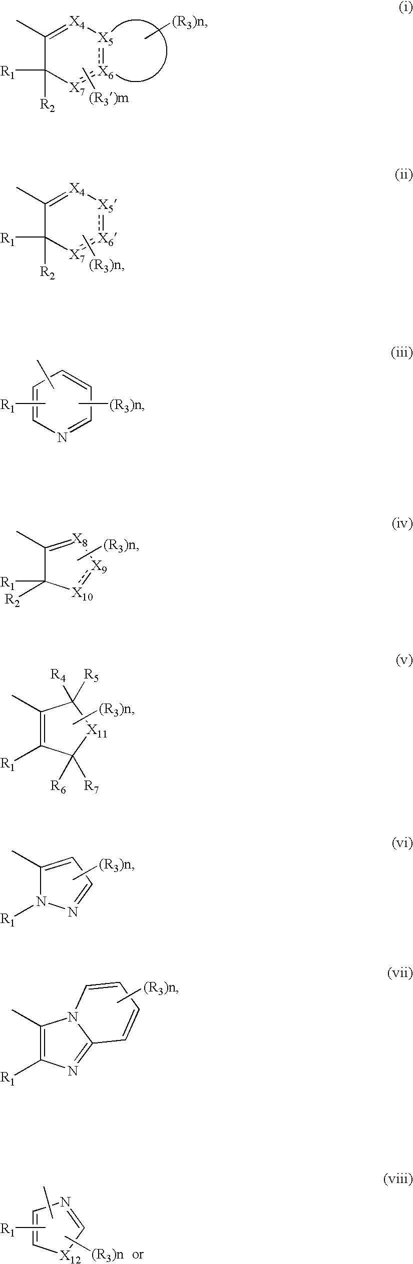

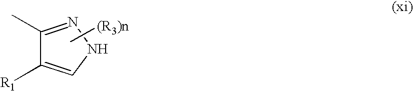

wherein
the formula:

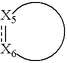

which partially constitutes the fused ring in the heterocyclic group represented by the formula (i):

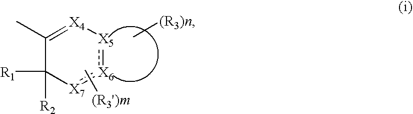

is a 5- to 7-membered ring which optionally contains, as a ring-constituting member, 1 to 3 members selected from O, N, S, SO and SO₂;
R₁ and R₂ are the same or different and each is
(1) a hydrogen atom;
(2) a C₁₋₆ alkyl group (preferably methyl, ethyl, propyl) optionally substituted by 1 to 3 C₆₋₁₂ aryl groups (preferably phenyl);
(3) a C₁₋₆ alkoxy group (preferably methoxy);
(4) a C₆₋₁₄ aryl group (preferably phenyl, naphthyl) optionally substituted by 1 to 3 substituents selected from
   (a) a halogen atom (preferably fluorine atom, chlorine atom),
   (b) a C₁₋₆ alkyl group (preferably methyl, ethyl, propyl, isopropyl) optionally substituted by 1 to 3 halogen atoms (preferably fluorine atom),
   (c) a C₁₋₆ alkoxy group (preferably methoxy) optionally substituted by 1 to 3 halogen atoms (preferably fluorine atom), and
   (d) a nitro group; or
(5) an aromatic heterocyclic group (preferably pyridyl);
R₃ is
(1) a C₁₋₆ alkyl group (preferably methyl, ethyl, propyl, butyl) optionally substituted by 1 to 5 substituents selected from
   (a) a halogen atom (preferably fluorine atom),
   (b) a C₁₋₆ alkoxy group (preferably ethoxy), and
   (c) a C₆₋₁₂ aryl group (preferably phenyl);
(2) a C₁₋₆ alkoxy group (preferably methoxy);
(3) a C₆₋₁₄ aryl group (preferably phenyl) optionally substituted by 1 to 3 halogen atoms (preferably fluorine atom, chlorine atom);
(4) a C₁₋₆ alkyl-carbonyl group (preferably acetyl) optionally substituted by 1 to 3 halogen atoms (preferably fluorine atom);
(5) a C₁₋₆ alkoxy-carbonyl group (preferably ethoxycarbonyl);
(6) a C₆₋₁₄ aryl-carbonyl group (preferably benzoyl);
(7) a C₁₋₆ alkylsulfonyl group (preferably methylsulfonyl);
(8) an aromatic heterocyclic group (preferably pyridyl);
(9) a halogen atom (preferably chlorine atom, bromine atom, iodine atom);
(10) a cyano group;
(11) an amino group optionally substituted by 1 or 2 C₁₋₆ alkyl groups (preferably methyl); or
(12) a carbamoyl group optionally substituted by 1 or 2 C₁₋₆ alkyl groups (preferably methyl);
R₄ and R₅ are hydrogen atoms, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group;
X₄ is N or CH;
X₅ is N or C;
X₆ is C;
X₅′ is N;
X₆′ is CH;
X₇ is O, S, SO or SO₂;
X₈ is N;
X₉ is NH;
X₁₀ is CH₂;
X₁₁ is O or NH;
X₁₂ is S;
X₅ [symbol] X₆ is X₅—X₆ or X₅═X₆;
X₆ [symbol] X₇ is X₆—X₇;
X₅′ [symbol] X₆′ is X₅′—X₆′ or X₅′═X₆′;
X₆′ [symbol] X₇ is X₆′—X₇;
X₉ [symbol] X₁₀ is X₉—X₁₀;
m is 0; and
n is 0, 1 or 2.

Of compounds (Ia) and (I), specifically, the following compound;
6-(7-phenyl-7H-[1,2,4]triazolo[3,4-b][1,3,4]thiadiazin-6-yl)-2H-1,4-benzoxazin-3(4H)-one (Example 3),
6-[2-(4-fluorophenyl)-2H-thiochromen-3-yl]-2H-1,4-benzoxazin-3(4H)-one (Example 158),
3-(3-oxo-3,4-dihydro-2H-1,4-benzoxazin-6-yl)-2-phenyl-2H-thiochromene-7-carbonitrile (Example 130),
6-(2-amino-6-phenyl-6H-1,3-thiazin-5-yl)-2H-1,4-benzoxazin-3(4H)-one (Example 24),
6-[7-(2-chlorophenyl)-7H-[1,2,4]triazolo[3,4-b][1,3,4]thiadiazin-6-yl]-2H-1,4-benzoxazin-3(4H)-one (Example 34),
8-fluoro-6-[7-(4-fluorophenyl)-7H-[1,2,4]triazolo[3,4-b][1,3,4]thiadiazin-6-yl]-2H-1,4-benzoxazin-3(4H)-one (Example 195),
6-[7-(4-fluorophenyl)-7H-[1,2,4]triazolo[3,4-b][1,3,4]thiadiazin-6-yl]-8-methyl-2H-1,4-benzoxazin-3(4H)-one (Example 185),
8-chloro-6-[7-(4-fluorophenyl)-7H-imidazo[2,1-b][1,3]thiazin-6-yl]-2H-1,4-benzoxazin-3(4H)-one (Example 118),
3-(4-fluorophenyl)-4-(3-oxo-3,4-dihydro-2H-benzo[b][1,4]oxazin-6-yl)-1-phenyl-1H-pyrrole-2,5-dione (Example 65),
6-(1-o-tolyl-3-(trifluoromethyl)-1H-pyrazol-5-yl)-2H-benzo[b][1,4]oxazin-3(4H)-one (Example 82),
6-(1-(4-fluoro-2-methylphenyl)-3-(trifluoromethyl)-1H-pyrazol-5-yl)-2H-benzo[b][1,4]oxazin-3(4H)-one (Example 107),
8-fluoro-6-(1-(4-fluoro-2-methylphenyl)-3-(trifluoromethyl)-1H-pyrazol-5-yl)-2H-benzo[b][1,4]oxazin-3(4H)-one (Example 108),
6-(1-(4-fluoro-2-methylphenyl)-3-(trifluoromethyl)-1H-pyrazol-5-yl)-8-methyl-2H-benzo[b][1,4]oxazin-3(4H)-one (Example 110),
6-(1,3-dimethyl-4-phenyl-1H-pyrazol-5-yl)-2H-benzo[b][1,4]oxazin-3(4H)-one (Example 288),
6-(1-(4-chloro-2-methylphenyl)-3-(trifluoromethyl)-1H-pyrazol-5-yl)-2H-benzo[b][1,4]oxazin-3(4H)-one (Example 86),
6-(1-(2,5-dimethylphenyl)-3-(trifluoromethyl)-1H-pyrazol-5-yl)-2H-benzo[b][1,4]oxazin-3(4H)-one (Example 93),
6-[1-(4-fluoro-2-methylphenyl)-3-(trifluoromethyl)-1H-pyrazol-5-yl]-8-methyl-2H-pyrido[3,2-b][1,4]oxazin-3(4H)-one (Example 255), and
6-(3-(1,1-difluoroethyl)-1-(4-fluoro-2-methylphenyl)-1H-pyrazol-5-yl)-8-methyl-2H-benzo[b][1,4]oxazin-3(4H)-one (Example 310),
   and salts thereof are more preferable.

Each type is explained in the following.

### Type (i)

As preferable examples of the heterocyclic group of type (i), heterocyclic groups represented by the formulas:

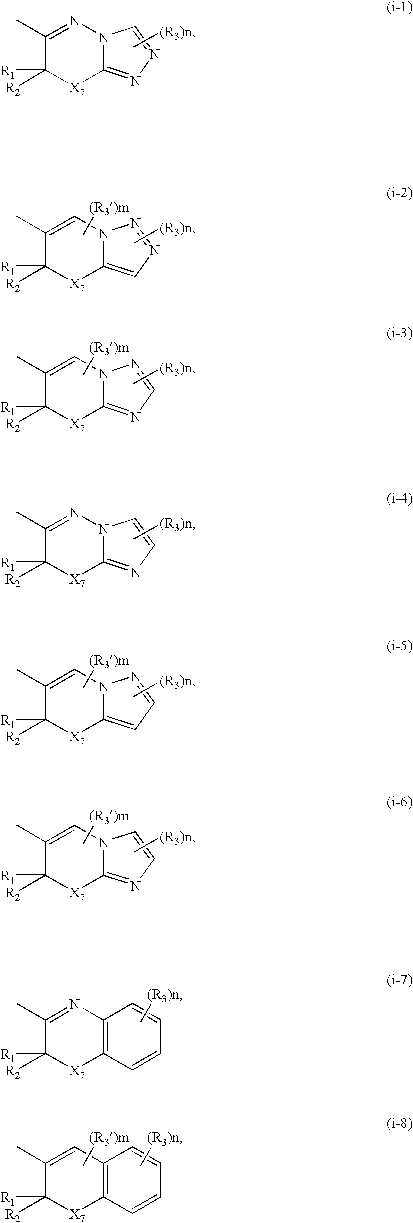

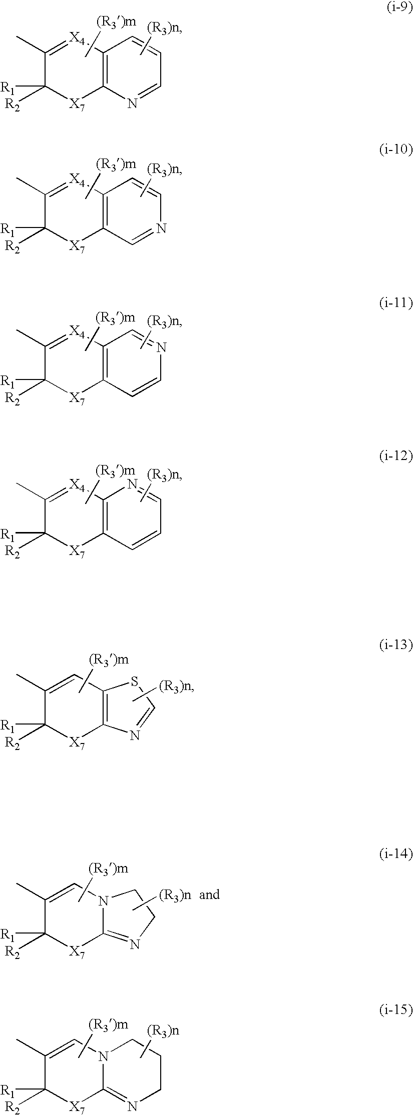

wherein each symbol is as defined above, can be mentioned. Of these, heterocyclic groups represented by the formulas:

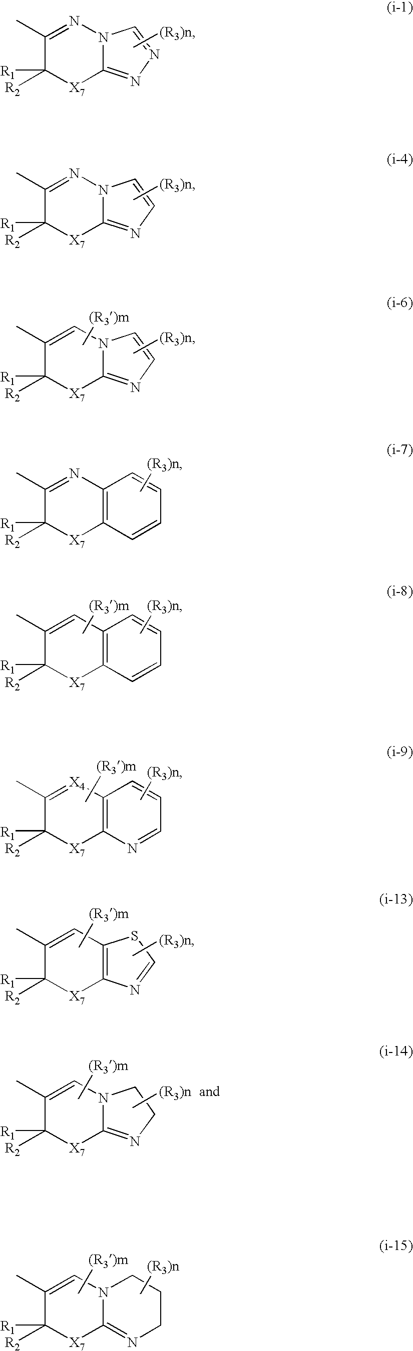

wherein each symbol is as defined above, are preferable.

As other preferable examples, heterocyclic groups represented by the formulas:

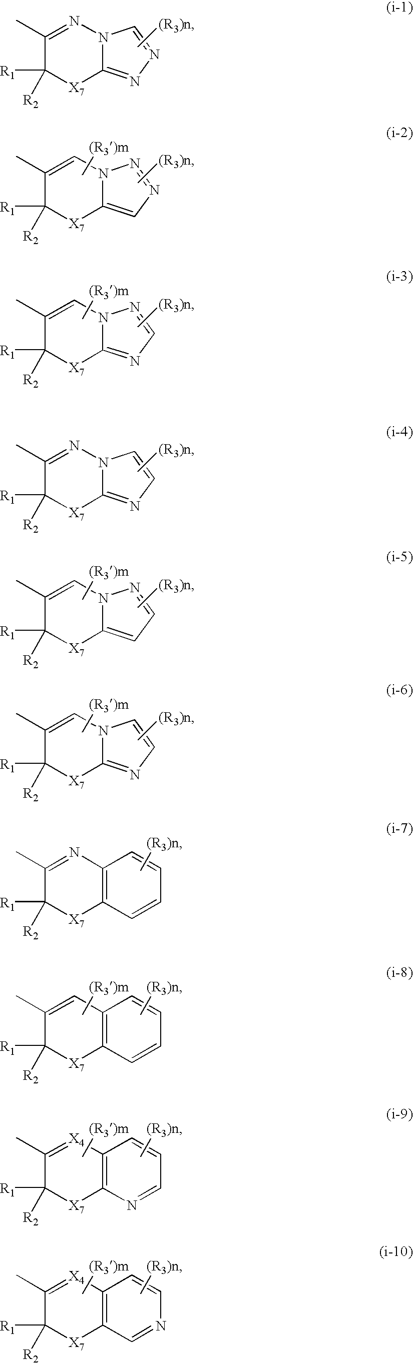

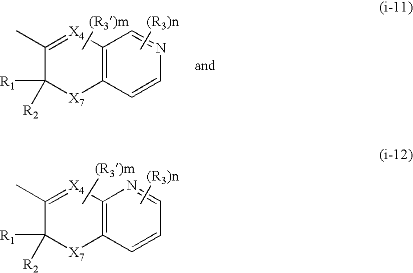

wherein each symbol is as defined above, can be mentioned. Of these, heterocyclic groups represented by the formulas:

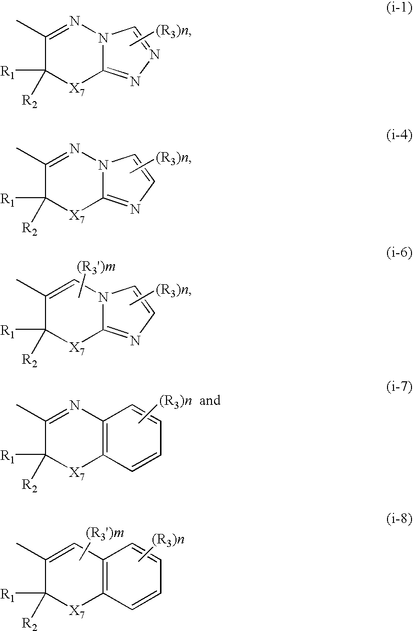

wherein each symbol is as defined above, are preferable.

Particularly, heterocyclic groups represented by the formulas:

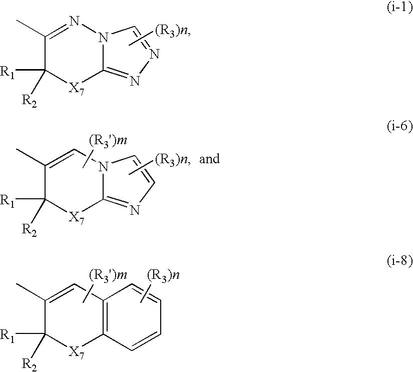

wherein each symbol is as defined above, are preferable.

In the above-mentioned formulas (i-1)-(i-15), preferably,

R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;

### X₄ is CH or N;

### X₇ is O, S, SO or SO₂;

m is an integer of 0 to 3; and
n is an integer of 0 to 4.

More preferably,

R₁ and R₂ are the same or different and each is a hydrogen atom, an optionally substituted aliphatic chain hydrocarbon group, an optionally substituted hydroxy group or an optionally substituted cyclic group;

R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a cyano group, an acyl group or an optionally substituted cyclic group;

### X₄ is CH or N;

### X₇ is O, S, SO or SO₂;

m is 0; and
n is 0 or 1.

Furthermore preferably,

R₁ and R₂, are the same or different and each is
(1) a hydrogen atom;
(2) a C₁₋₆ alkyl group (preferably methyl, propyl) optionally substituted by 1 to 3 C₆₋₁₂ aryl groups (preferably phenyl);
(3) a C₁₋₆ alkoxy group (preferably methoxy);
(4) a C₆₋₁₄ aryl group (preferably phenyl) optionally substituted by 1 to 3 substituents selected from

(a) a halogen atom (preferably fluorine atom, chlorine atom, bromine atom),

(b) a C₁₋₆ alkyl group (preferably methyl),

(c) a cyano group,

(d) an amino group, and

(e) a nitro group; or

(5) an aromatic heterocyclic group (preferably pyridyl, thiazolyl);

### R₃ is

(1) a C₁₋₆ alkyl group (preferably methyl, ethyl, propyl, isopropyl) optionally substituted by 1 to 3 substituents selected from

(a) a halogen atom (preferably fluorine atom),

(b) a hydroxy group,

(c) a C₁₋₆ alkoxy group (preferably ethoxy), and

(d) a C₁₋₆ alkyl-carbonyloxy group (preferably acetyloxy);

(2) a C₁₋₆ alkoxy group (preferably methoxy, ethoxy) optionally substituted by 1 to 3 hydroxy groups;
(3) a C₁₋₆ alkoxy-carbonyl group (preferably methoxycarbonyl);
(4) a C₁₋₆ alkylsulfonyl group (preferably methylsulfonyl);
(5) an aromatic heterocyclic group (preferably pyridyl);
(6) a halogen atom (preferably fluorine atom, chlorine atom, iodine atom);
(7) a hydroxy group;
(8) a carboxyl group;
(9) a cyano group;
(10) an amino group optionally substituted by 1 or 2 C₁₋₁₀ alkyl groups (preferably ethyl, propyl, tert-butyl, heptyl) optionally substituted by 1 to 3 substituents selected from

(a) a hydroxy group,

(b) a C₁₋₆ alkoxy group (preferably methoxy),

(c) an amino group optionally substituted by 1 or 2 C₁₋₆ alkyl groups (preferably methyl), and

(d) a C₆₋₁₂ aryloxy group (preferably phenoxy); or

(11) carbamoyl group optionally substituted by 1 or 2 C₁₋₆ alkyl groups (preferably methyl);

### X₄ is N or CH;

### X₇ is O, S, SO or SO₂;

m is 0; and
n is 0 or 1;
and in the above-mentioned embodiment, preferably,

### A is —CH₂—O—, —O— or —CH₂—S—;

### R′ is

(1) a C₁₋₆ alkyl group (preferably methyl); or
(2) a halogen atom (preferably fluorine atom, chlorine atom);
k is 0;
l is 0 or 1;
Xₐ is CH or N;
X_{b} is CH or N; and

### X_{c} is CH.

Particularly preferably,

R₁ and R₂ are the same or different and each is
(1) a hydrogen atom;
(2) a C₁₋₆ alkyl group (preferably methyl, propyl) optionally substituted by 1 to 3 C₆₋₁₂ aryl groups (preferably phenyl);
(3) a C₁₋₆ alkoxy group (preferably methoxy);
(4) a C₆₋₁₄ aryl group (preferably phenyl) optionally substituted by 1 to 3 halogen atoms (preferably fluorine atom, chlorine atom); or
(5) an aromatic heterocyclic group (preferably pyridyl);

### R₃ is

(1) a C₁₋₆ alkyl group (preferably methyl, propyl) optionally substituted by 1 to 3 substituents selected from

(a) a halogen atom (preferably fluorine atom), and

(b) a C₁₋₆ alkoxy group (preferably ethoxy);

(2) a C₁₋₆ alkoxy group (preferably methoxy);
(3) a halogen atom (preferably chlorine atom, iodine atom); or
(4) a cyano group;

### X₄ is N or CH;

### X₇ is O, S, SO or SO₂;

m is 0; and
n is 0 or 1;
and in the above-mentioned embodiment, preferably,

### A is —CH₂—O— or —O—;

R′ is a halogen atom (preferably fluorine atom, chlorine atom);
k is 0;
l is 0 or 1;

### Xₐ is CH;

### X_{b} is CH; and

### X_{c} is CH.

### Type (ii)

In type (ii), preferably,

R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group;

### X₄ is CH or N;

X₅′ and X₆′ are the same or different and each is CH₂, CH, NH or N;

### X₇ is CH₂, O, S, SO or SO₂;

[symbol] is a single bond or a double bond;
provided that
when X₅′ [symbol] X₆′ is X₅′═X₆′, then X₆′ [symbol] X₇ should be X₆′—X₇;
n is an integer of 0 to 4; and
at least one of R₁ and R₂ should be an optionally substituted aryl group or an optionally substituted heteroaryl group.

More preferably,

R₁ and R₂ are the same or different and each is a hydrogen atom or an optionally substituted cyclic group;
R₃ is an optionally substituted aliphatic chain hydrocarbon group, an optionally substituted amino group, an oxo group, an optionally substituted imino group, an acyl group or an optionally substituted cyclic group;

### X₄ is CH or N;

X₅′ and X₆′ are the same or different and each is CH₂, CH, NH or N;

### X₇ is CH₂ or S;

[symbol] is a single bond or a double bond;
provided that
when X₅′ [symbol] X₆′ is X₅′═X₆′, then X₆′ [symbol] X₇ should be X₆′—X₇;
n is an integer of 0 to 2; and
at least one of R₁ and R₂ should be an optionally substituted aryl group or an optionally substituted heteroaryl group.

Furthermore preferably,

R₁ and R₂ are the same or different and each is
(1) a hydrogen atom; or
(2) a C₆₋₁₄ aryl group (preferably phenyl) optionally substituted by 1 to 3 halogen atoms (preferably fluorine atom);

### R₃ is

(1) a C₁₋₆ alkyl group (preferably methyl, ethyl) optionally substituted by 1 to 3 substituents selected from

(a) a C₁₋₆ alkoxy-carbonyl group (preferably methoxycarbonyl), and

(b) a hydroxy group;

(2) a C₆₋₁₄ aryl group (preferably phenyl);
(3) a C₁₋₆ alkyl-carbonyl group (preferably acetyl);
(4) a C₁₋₆ alkylsulfonyl group (preferably methylsulfonyl);
(5) an amino group optionally substituted by 1 or 2 C₁₋₆ alkyl groups (preferably methyl, ethyl);
(6) an imino group; or
(7) an oxo group;

### X₄ is N or CH;

### X₅′ is N, NH or CH;

### X₆′ is N, CH or CH₂;

### X₇ is CH or S;

### X₅′X₆′ is X₅′—X₆′ or X₅′═X₆′;

### X₆′X₇ is X₆′—X₇;

n is 0, 1 or 2; and
at least one of R₁ and R₂ should be an optionally substituted aryl group;
and in the above-mentioned embodiment, preferably,

### A is —CH₂—O—;

R′ is a C₁₋₆ alkyl group (preferably methyl);
k is 0;
l is 0 or 1;

### Xₐ is CH;

### X_{b} is CH; and

### X_{c} is CH.

Particularly preferably,

R₁ and R₂ are the same or different and each is
(1) a hydrogen atom; or
(2) a C₆₋₁₄ aryl group (preferably phenyl) optionally substituted by 1 to 3 halogen atoms (preferably fluorine atom);
R₃ is an amino group;

### X₄ is CH;

### X₅′ is N;

### X₆′ is CH;

### X₇ is S;

n is 1; and
at least one of R₁ and R₂ should be an optionally substituted aryl group;
and in the above-mentioned embodiment, preferably,

### A is —CH₂—O—;

k is 0;
l is 0;

### Xₐ is CH;

### X_{b} is CH; and

### X_{c} is CH.

Type (iii)

In type (iii), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
n is an integer of 0 to 3;
the carbon atom to which the group represented by the formula:

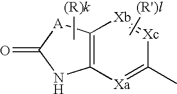

is bonded and the carbon atom to which R₁ is bonded should be adjacent to each other; and
when the group represented by the formula:
—X₁ [symbol] X₂ [symbol] X₃— is —O—, —CH₂—O—, —CH₂—S— or —CH═CH—, then R₁ should not be a halogen atom and trifluoromethyl.

More preferably,

R₁ is an optionally substituted cyclic group;
n is 0; and
the carbon atom to which the group represented by the formula:

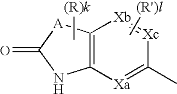

is bonded and the carbon atom to which R₁ is bonded should be adjacent to each other.

Furthermore preferably,

R₁ is C₆₋₁₄ aryl group (preferably phenyl);
n is 0; and
the carbon atom to which the group represented by the formula:

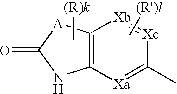

is bonded and the carbon atom to which R₁ is bonded should be adjacent to each other; and in the above-mentioned embodiment, preferably,

### A is —CH₂—O—;

k is 0;
l is 0;

### Xₐ is CH;

### X_{b} is CH; and

### X_{c} is CH.

### Type (iv)

In type (iv), preferably,

R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;

### X₈ is CH or N;

### X₉ is CH₂, CH, NH, N or O;

### X₁₀ is CH₂, CH, NH or N;

n is an integer of 0 to 4; and
at least one of R₁ and R₂ should be an optionally substituted aryl group or an optionally substituted aromatic heterocyclic group.

More preferably,

R₁ and R₂ are the same or different and each is a hydrogen atom or an optionally substituted cyclic group;
R₃ is an optionally substituted aliphatic chain hydrocarbon group, an acyl group or an optionally substituted cyclic group;

### X₈ is N;

### X₉ is NH;

### X₁₀ is CH₂;

n is 0 or 1; and
at least one of R₁ and R₂ should be an optionally substituted aryl group or an optionally substituted aromatic heterocyclic group.

Furthermore preferably,

R₁ and R₂ are the same or different and each is
(1) a hydrogen atom; or
(2) a C₆₋₁₄ aryl group (preferably phenyl) optionally substituted by 1 to 3 halogen atoms (preferably fluorine atom);

### R₃ is

(1) a C₁₋₆ alkyl group (preferably methyl, ethyl, butyl) optionally substituted by 1 to 3 substituents selected from

(a) a halogen atom (preferably fluorine atom),

(b) a hydroxy group, and

(c) a C₆₋₁₂ aryl group (preferably phenyl);

(2) a C₆₋₁₄ aryl group (preferably phenyl) optionally substituted by 1 to 3 halogen atoms (preferably fluorine atom, chlorine atom);
(3) a C₁₋₆ alkyl-carbonyl group (preferably acetyl) optionally substituted by 1 to 3 halogen atoms (preferably fluorine atom);
(4) a C₆₋₁₄ aryl-carbonyl group (preferably benzoyl); or
(5) a C₁₋₆ alkylsulfonyl group (preferably methylsulfonyl);

### X₈ is N;

### X₉ is NH;

### X₁₀ is CH₂;

n is 0 or 1; and
at least one of R₁ and R₂ should be an optionally substituted aryl group; and in the above-mentioned embodiment, preferably,

### A is CH₂—O—;

k is 0;
l is 0;

### Xₐ is CH;

### X_{b} is CH; and

### X_{c} is CH.

Particularly preferably,

R₁ and R₂ are the same or different and each is
(1) a hydrogen atom; or
(2) a C₆₋₁₄ aryl group (preferably phenyl) optionally substituted by 1 to 3 halogen atoms (preferably fluorine atom);

### R₃ is

(1) a C₁₋₆ alkyl group (preferably methyl, ethyl, butyl) optionally substituted by 1 to 3 substituents selected from

(a) a halogen atom (preferably fluorine atom), and

(b) a C₆₋₁₂ aryl group (preferably phenyl);

(2) a C₆₋₁₄ aryl group (preferably phenyl) optionally substituted by 1 to 3 halogen atoms (preferably fluorine atom, chlorine atom);
(3) a C₁₋₆ alkyl-carbonyl group (preferably acetyl) optionally substituted by 1 to 3 halogen atoms (preferably fluorine atom);
(4) a C₆₋₁₄ aryl-carbonyl group (preferably benzoyl); or
(5) a C₁₋₆ alkylsulfonyl group (preferably methylsulfonyl);

### X₈ is N;

### X₉ is NH;

### X₁₀ is CH₂;

n is 0 or 1; and
at least one of R₁ and R₂ should be an optionally substituted aryl group;
and in the above-mentioned embodiment, preferably,

### A is CH₂—O—;

k is 0;
l is 0;

### Xₐ is CH;

### X_{b} is CH; and

### X_{c} is CH.

### Type (v)

As preferable examples of the heterocyclic group of type (v), heterocyclic groups represented by the formulas:

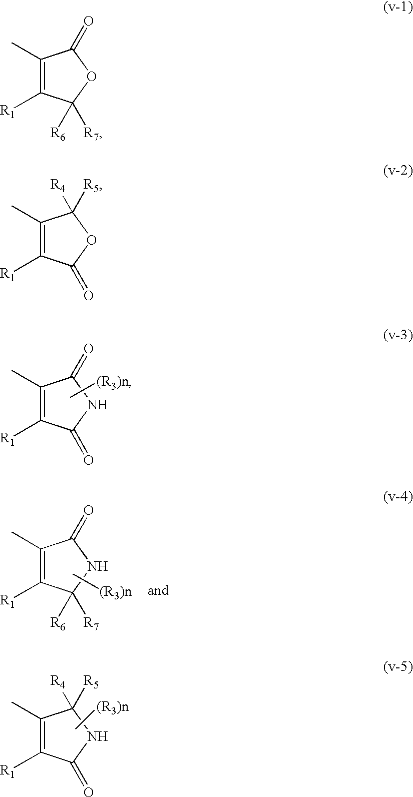

wherein each symbol is as defined above, can be mentioned. Of these,

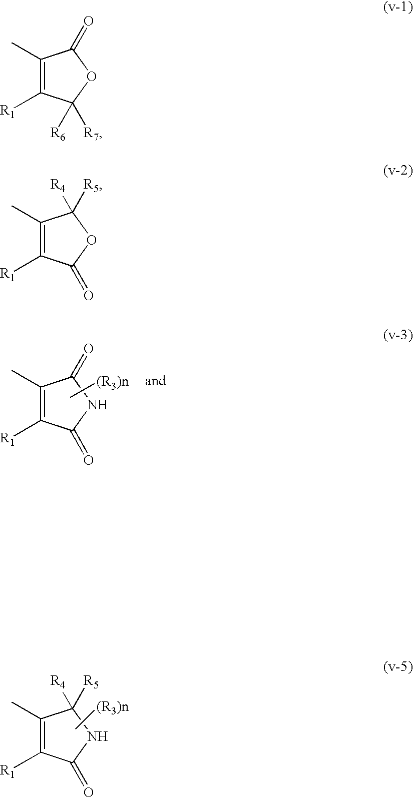

wherein each symbol is as defined above, are preferable.

In the above-mentioned formulas (v-1)-(v-5), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, an optionally substituted mercapto group, an acyl group or an optionally substituted cyclic group;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or R₆ and R₇ in combination optionally form an oxo group;
provided that at least one of a pair of R₄ and R₅ and a pair of R₆ and R₇ should form an oxo group;
n is 0 or 1; and
when the group represented by the formula:
—X₁ [symbol] X₂ [symbol] X₃— is —CH₂—O—, and the heterocyclic group represented by the formula:

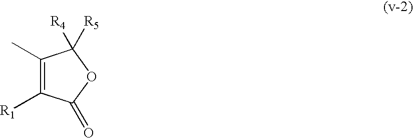

is a heterocyclic group represented by the formula:

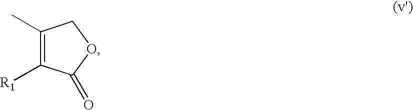

then R₁ should not be phenyl, 4-methoxyphenyl, 3,4-dimethoxyphenyl and 4-chlorophenyl.

More preferably,

R₁ is an optionally substituted cyclic group;
R₃ is an optionally substituted aliphatic chain hydrocarbon group or an optionally substituted cyclic group;
R₄ and R₅ are hydrogen atoms, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group;
n is 0 or 1; and
when the group represented by the formula:
—X₁ [symbol] X₂ [symbol] X₃— is —CH₂—O—, and the heterocyclic group represented by the formula:

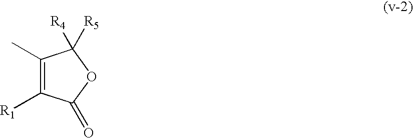

is a heterocyclic group represented by the formula:

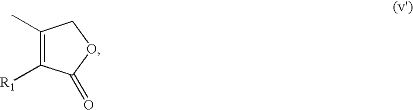

then R₁ should not be phenyl, 4-methoxyphenyl, 3,4-dimethoxyphenyl and 4-chlorophenyl.

Furthermore preferably,

R₁ is a C₆₋₁₄ aryl group (preferably phenyl) optionally substituted by 1 to 3 halogen atoms (preferably fluorine atom, chlorine atom);

### R₃ is

(1) a C₁₋₆ alkyl group (preferably ethyl) by optionally substituted 1 to 3 halogen atoms (preferably fluorine atom);
(2) a C₆₋₁₄ aryl group (preferably phenyl) by optionally substituted by 1 to 3 halogen atoms (preferably fluorine atom); or
(3) an aromatic heterocyclic group (preferably pyridyl);
R₄ and R₅ are hydrogen atoms, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group; and
n is 0 or 1;
and in the above-mentioned embodiment, preferably,

### A is —CH₂—O— or —CH₂—S—;

k is 0;
l is 0;

### Xₐ is CH;

### X_{b} is CH; and

### X_{c} is CH,

provided that when the group represented by the formula:
—X₁ [symbol] X₂ [symbol] X₃— is —CH₂—O—, and the heterocyclic group represented by the formula:

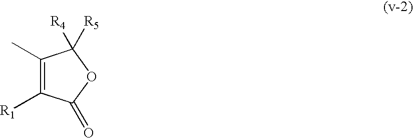

is a heterocyclic group represented by the formula:

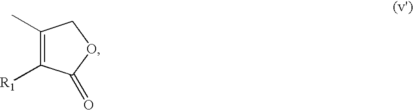

then R₁ should not be phenyl and 4-chlorophenyl.

Particularly preferably,

R₁ is a C₆₋₁₄ aryl group (preferably phenyl) optionally substituted by 1 to 3 halogen atoms (preferably fluorine atom, chlorine atom);

### R₃ is

(1) a C₆₋₁₄ aryl group (preferably phenyl) optionally substituted by 1 to 3 halogen atoms (preferably fluorine atom); or
(2) an aromatic heterocyclic group (preferably pyridyl);
R₄ and R₅ are hydrogen atoms, or R₄ and R₅ in combination optionally form an oxo group;
R₆ and R₇ are hydrogen atoms, or R₆ and R₇ in combination optionally form an oxo group;
provided that at least one of a pair of R₄ and R₅ and a pair of R₆ and R₇ should form an oxo group; and
n is 0 or 1;
and in the above-mentioned embodiment, preferably,

### A is —CH₂—O— or —CH₂—S—;

k is 0;
l is 0;

### Xₐ is CH;

### X_{b} is CH; and

### X_{c} is CH,

provided that when the group represented by the formula:
—X₁ [symbol] X₂ [symbol] X₃— is —CH₂—O—, and the heterocyclic group represented by the formula:

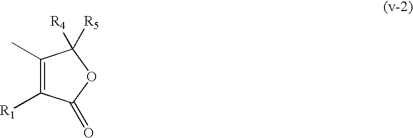

is a heterocyclic group represented by the formula:

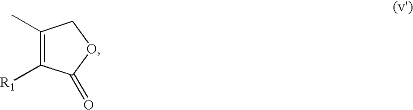

then R₁ should not be phenyl and 4-chlorophenyl.

### Type (vi)

In type (vi), preferably,

R₁ is an optionally substituted aliphatic chain hydrocarbon group, an optionally substituted amino group, an optionally esterified carboxyl group, an optionally substituted carbamoyl group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group; and
n is an integer of 0 to 2.

More preferably

R₁ is an optionally substituted aliphatic chain hydrocarbon group or an optionally substituted cyclic group;
R₃ is an optionally substituted aliphatic chain hydrocarbon group, an optionally substituted amino group, an optionally esterified carboxyl group, an optionally substituted carbamoyl group, a halogen atom, cyano group or an acyl group; and
n is an integer of 0 to 2.

Furthermore preferably,

### R₁ is

(1) a C₁₋₆ alkyl group (preferably methyl, ethyl) optionally substituted by 1 to 3 C₆₋₁₂ aryl groups (preferably phenyl) optionally substituted by 1 to 3 C₁₋₆ alkyl groups (preferably methyl);
(2) a C₆₋₁₄ aryl group (preferably phenyl, naphthyl) optionally substituted by 1 to 3 substituents selected from

(a) a halogen atom (preferably fluorine atom, chlorine atom, bromine atom),

(b) a C₁₋₆ alkyl group (preferably methyl, ethyl, propyl, isopropyl) optionally substituted by 1 to 3 halogen atoms (preferably fluorine atom),

(c) a C₁₋₆ alkoxy group (preferably methoxy) optionally substituted by 1 to 3 halogen atoms (preferably fluorine atom),

(d) a hydroxy group,

(e) a cyano group, and

(f) a nitro group; or

(3) an aromatic heterocyclic group (preferably pyridyl, thienyl) optionally substituted by 1 to 3 C₁₋₆ alkoxy-carbonyl groups (preferably methoxycarbonyl);

### R₃ is

(1) a C₁₋₆ alkyl group (preferably methyl, ethyl, propyl) optionally substituted by 1 to 5 substituents selected from

(a) a halogen atom (preferably fluorine atom),

(b) a hydroxy group, and

(c) a C₁₋₆ alkoxy-carbonyl group (preferably methoxycarbonyl);

(2) a C₂₋₆ alkenyl group (preferably vinyl) optionally substituted by 1 to 3 C₁₋₆ alkoxy-carbonyl groups (preferably methoxycarbonyl);
(3) a C₁₋₆ alkyl-carbonyl group (preferably acetyl);
(4) a C₁₋₆ alkoxy-carbonyl group (preferably ethoxycarbonyl);
(5) a halogen atom (preferably fluorine atom, chlorine atom, bromine atom, iodine atom);
(6) a cyano group;
(7) a amino group; or
(8) a carbamoyl group optionally substituted by 1 or 2 C₁₋₆ alkyl groups (preferably methyl); and
n is an integer of 0 to 2;
and in the above-mentioned embodiment, preferably,

### A is —CH₂—O—, —O—, —CH₂—S— or —CH₂—;

### R′ is

(1) a halogen atom (preferably fluorine atom, chlorine atom, bromine atom);
(2) a C₁₋₆ alkyl group (preferably methyl) optionally substituted by 1 to 3 hydroxy groups;
(3) a C₁₋₆ alkoxy group (preferably methoxy);
(4) an amino group; or
(5) a nitro group;
k is 0;
l is 0 or 1;

### Xₐ is CH or N;

### X_{b} is CH or N; and

### X_{c} is CH or N.

Particularly preferably,

### R₁ is

(1) a C₁₋₆ alkyl group (preferably methyl, ethyl) optionally substituted by 1 to 3 C₆₋₁₂ aryl groups (preferably phenyl);
(2) a C₆₋₁₄ aryl group (preferably phenyl, naphthyl) optionally substituted by 1 to 3 substituents selected from

(a) a halogen atom (preferably fluorine atom, chlorine atom),

(b) a C₁₋₆ alkyl group (preferably methyl, ethyl, propyl, isopropyl) optionally substituted by 1 to 3 halogen atoms (preferably fluorine atom),

(c) a C₁₋₆ alkoxy group (preferably methoxy) optionally substituted by 1 to 3 halogen atoms (preferably fluorine atom), and

(d) a nitro group; or

(3) an aromatic heterocyclic group (preferably pyridyl);

### R₃ is

(1) a C₁₋₆ alkyl group (preferably methyl, ethyl) optionally substituted by 1 to 5 halogen atoms (preferably fluorine atom);
(2) a C₁₋₆ alkoxy-carbonyl group (preferably ethoxycarbonyl);
(3) a halogen atom (preferably bromine atom);
(4) a cyano group; or
(5) a carbamoyl group optionally substituted by one or two C₁₋₆ alkyl groups (preferably methyl); and
n is 1 or 2;
and in the above-mentioned embodiment, preferably,

### A is —CH₂—O—;

### R′ is

(1) a halogen atom (preferably fluorine atom, chlorine atom); or
(2) a C₁₋₆ alkyl group (preferably methyl);
k is 0;
l is 0 or 1;

### Xₐ is CH;

### X_{b} is CH; and

### X_{c} is CH.

Type (vii)

In type (vii), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group; and
n is an integer of 0 to 4.

More preferably,

R₁ is an optionally substituted cyclic group; and
n is 0.

Furthermore preferably,

R₁ is a C₆₋₁₄ aryl group (preferably phenyl); and
n is 0;
and in the above-mentioned embodiment, preferably,

### A is —CH₂—O—;

k is 0;
l is 0;

### Xₐ is CH;

### X_{b} is CH; and

### X_{c} is CH.

Type (viii)

In type (viii), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;

### X₁₂ is O or S;

n is 0 or 1; and
the carbon atom to which the group represented by the formula:

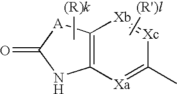

is bonded and the carbon atom to which R₁ is bonded should be adjacent to each other.

More preferably,

R₁ is an optionally substituted cyclic group;
R₃ is an optionally substituted aliphatic chain hydrocarbon group or an optionally substituted amino group;

### X₁₂ is S;

n is 1; and
the carbon atom to which the group represented by the formula:

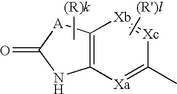

is bonded and the carbon atom to which R₁ is bonded should be adjacent to each other.

Furthermore preferably,

R₁ is a C₆₋₁₄ aryl group (preferably phenyl);

### R₃ is

(1) a C₁₋₆ alkyl group (preferably methyl); or
(2) an amino group optionally substituted by 1 or 2 C₁₋₆ alkyl groups (preferably methyl);

### X₁₂ is S;

n is 1; and
the carbon atom to which the group represented by the formula:

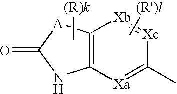

is bonded and the carbon atom to which R₁ is bonded should be adjacent to each other;
and in the above-mentioned embodiment, preferably,

### A is —CH₂—O—;

k is 0;
l is 0;

### Xₐ is CH;

### X_{b} is CH; and

### X_{c} is CH.

### Type (ix)

In type (ix), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
n is an integer of 0 to 2;
when the group represented by the formula:
—X₁ [symbol] X₂ [symbol] X₃— is —CH₂—O—, then R₁ should not be an optionally substituted 2-pyridyl; and
when the group represented by the formula:
—X₁ [symbol] X₂ [symbol] X₃— is —CH₂—O—, and R₁ is an optionally substituted phenyl, then NH— group in the pyrazole ring of the heterocyclic group represented by the formula:

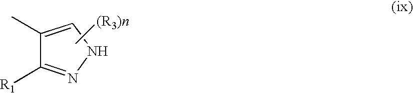

should be substituted by R₃.

More preferably,

R₁ is an optionally substituted cyclic group;
R₃ is an optionally substituted aliphatic chain hydrocarbon group;
n is 1;
when the group represented by the formula:
—X₁ [symbol] X₂ [symbol] X₃— is —CH₂—O—, then R₁ should not be an optionally substituted 2-pyridyl; and
when the group represented by the formula:
—X₁ [symbol] X₂ [symbol] X₃— is —CH₂—O—, and R₁ is an optionally substituted phenyl, then NH— group in the pyrazole ring of the heterocyclic group represented by the formula:

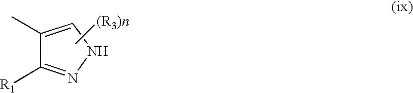

should be substituted by R₃.

Furthermore preferably,

R₁ is a C₆₋₁₄ aryl group (preferably phenyl) optionally substituted by 1 to 3 substituents selected from

(a) a halogen atom (preferably fluorine atom), and

(b) a C₁₋₆ alkyl group (preferably methyl);

R₃ is a C₁₋₆ alkyl group (preferably methyl); and
n is 1;
and in the above-mentioned embodiment, preferably,

### A is —CH₂—O—;

k is 0;
l is 0;

### Xₐ is CH; and

### X_{b} is CH;

### X_{c} is CH,

provided that when the group represented by the formula:
—X₁ [symbol] X₂ [symbol] X₃— is —CH₂—O—, and R₁ is an optionally substituted phenyl, then NH— group in the pyrazole ring of the heterocyclic group represented by the formula:

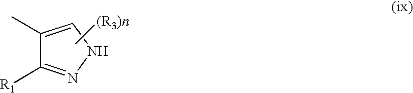

should be substituted by R₃.

### Type (x)

In type (x), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
n is an integer of 0 to 2; and
when the group represented by the formula:
—X₁ [symbol] X₂ [symbol] X₃— is —NH— or —CH₂—NH—, then R₁ should not be an alkyl group.

### Type (xi)

In type (xi), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
n is 0 or 1; and
when the group represented by the formula:
—X₁ [symbol] X₂ [symbol] X₃— is —CH₂—O—, then R₁ should be an optionally substituted aryl group or an optionally substituted heteroaryl group.

More preferably,

R₁ is an optionally substituted cyclic group;
R₃ is an optionally substituted aliphatic chain hydrocarbon group;
n is 0 or 1; and
when the group represented by the formula:
—X₁ [symbol] X₂ [symbol] X₃— is —CH₂—O—, then R₁ should be an optionally substituted aryl group or an optionally substituted heteroaryl group.

Furthermore preferably,

R₁ is a C₆₋₁₄ aryl group (preferably phenyl);
R₃ is a C₁₋₆ alkyl group (preferably ethyl) optionally substituted by 1 to 3 halogen atoms (preferably fluorine atom); and
n is 0 or 1;
and in the above-mentioned embodiment, preferably,

### A is —CH₂—O—;

k is 0;
l is 0;

### Xₐ is CH;

### X_{b} is CH; and

### X_{c} is CH.

Type (xii)

In type (xii), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, an optionally substituted mercapto group, an acyl group or an optionally substituted cyclic group;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or R₄ and
R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or R₆ and R₇ in combination optionally form an oxo group;
provided that at least one of a pair of R₄ and R₅ and a pair of R₆ and R₇ should form an oxo group;

### X₁₁ is O or S; and

n is 0 or 1.

More preferably,

R₁ is an optionally substituted cyclic group;
R₄ and R₅ in combination optionally form an oxo group;
R₆ and R₇ are hydrogen atoms;

### X₁₁ is O; and

n is 0.

Furthermore preferably,

R₁ is a C₆₋₁₄ aryl group (preferably phenyl);
R₄ and R₅ in combination optionally form an oxo group;
R₆ and R₇ are hydrogen atoms;

### X₁₁ is O; and

n is 0;
and in the above-mentioned embodiment, preferably,
k is 0;
l is 0;

### A is —CH₂—O—;

### Xₐ is CH;

### X_{b} is CH; and

### X_{c} is CH.

Type (xiii)

In type (xiii), preferably,

R₁ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
n is 1 or 2; and
when the group represented by the formula:
—X₁ [symbol] X₂ [symbol] X₃— is —S— or —CH₂—O—, then R₁ should not be a halogen atom.

More preferably

R₁ is an optionally substituted cyclic group;
R₃ is an optionally substituted aliphatic chain hydrocarbon group;
n is 1 or 2; and
when the group represented by the formula:
—X₁ [symbol] X₂ [symbol] X₃— is —S— or —CH₂—O—, then R₁ should not be a halogen atom.

Furthermore preferably,

R₁ is a C₆₋₁₄ aryl group (preferably phenyl) optionally substituted by 1 to 3 substituents selected from

(a) a halogen atom (preferably fluorine atom, chlorine atom),

(b) a C₁₋₆ alkyl group (preferably methyl) optionally substituted by 1 to 3 halogen atoms (preferably fluorine atom),

(c) a hydroxy group, and

(d) a C₁₋₆ alkoxy group (preferably methoxy);

R₃ is a C₁₋₆ alkyl group (preferably methyl, ethyl, isopropyl) optionally substituted by 1 to 3 halogen atoms (preferably fluorine atom); and
n is 1 or 2;
and in the above-mentioned embodiment, preferably,

### A is —CH₂—O—;

R′ is a halogen atom (preferably fluorine atom, chlorine atom);
k is 0;
l is 0 or 1;

### Xₐ is CH or N;

### X_{b} is CH; and

### X_{c} is CH.

As the salts of compound (I), compound (Ia), compound (I′) and compound (Ia′) (hereinafter, these are also collectively referred to as compound (I)), for example, metal salts, ammonium salts, salts with organic base, salts with inorganic acid, salts with organic acid, salts with basic or acidic amino acid and the like can be mentioned. As preferable examples of the metal salt, for example, alkali metal salts such as sodium salt, potassium salt and the like; alkaline earth metal salts such as calcium salt, magnesium salt, barium salt and the like; aluminum salt and the like can be mentioned. As preferable examples of the salts with organic base, for example, salts with trimethylamine, triethylamine, pyridine, picoline, 2,6-lutidine, ethanolamine, diethanolamine, triethanolamine, tromethamine[tris(hydroxymethyl)methylamine], t-butylamine, cyclohexylamine, dicyclohexylamine, N,N′-dibenzylethylenediamine and the like can be mentioned. As preferable examples of salts with inorganic acid, for example, salts with hydrochloric acid, hydrobromic acid, nitric acid, sulfuric acid, phosphoric acid and the like can be mentioned. As preferable examples of the salts with organic acid, for example, salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like can be mentioned. As preferable examples of the salts with basic amino acid, for example, salts with arginine, lysine, ornithine and the like can be mentioned. As preferable examples of the salts with acidic amino acid, for example, salts with aspartic acid, glutamic acid and the like can be mentioned.

Of these, pharmaceutically acceptable salts are preferable. When a compound contains an acidic functional group, for example, inorganic salts such as alkali metal salts (e.g., sodium salt, potassium salt etc.), alkaline earth metal salts (e.g., calcium salt, magnesium salt, barium salt etc.) and the like, ammonium salt and the like can be mentioned. And when a compound contains a basic functional group, for example, salts with inorganic acid such as hydrochloric acid, hydrobromic acid, nitric acid, sulfuric acid, phosphoric acid and the like, and salts with organic acid such as acetic acid, phthalic acid, fumaric acid, oxalic acid, tartaric acid, maleic acid, citric acid, succinic acid, methanesulfonic acid, benzenesulfonic acid, p-toluenesulfonic acid and the like can be mentioned.

The production methods of compound (I) are shown in the following.

Compounds (Ia′) and (I′) [particularly, compounds (Ia) and (I)] can be produced by a method known per se (e.g., the method described in Katritzky, A. R, COMPREHENSIVE HETEROCYCLIC CHEMISTRY, PERGAMON PRESS, 1984, vol. 3, pp. 1014-1037, vol. 5, p 273-290 and the like) or a method analogous thereto. In addition, Compounds (Ia′) and (I′) [particularly, compounds (Ia) and (I)] can be produced, for example, by the method shown in the following. Each compound described in the following Reaction scheme may form a salt as long as it does not inhibit the reaction, and as such salt, salts similar to the salts of compound (I) can be mentioned.

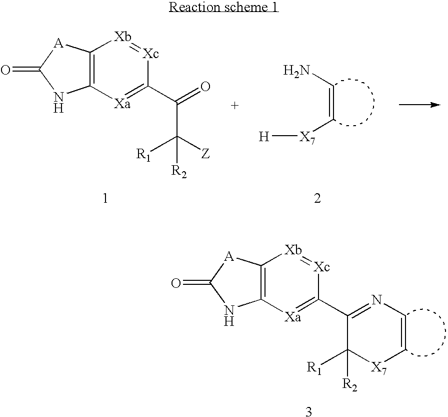

wherein Z is a leaving group, and other symbols are as defined above.

Compound (3) can be produced by subjecting compound (1) and compound (2) to a fused cyclization reaction.

The fused cyclization reaction can be carried out without solvent or in an inert solvent.

As the solvent, for example, toluene, benzene, xylene, methanol, ethanol, propanol, isopropanol, n-butanol, acetone, chloroform, dichloromethane, 1,2-dichloroethane, tetrahydrofuran, diethyl ether, acetonitrile, hexane, ethyl acetate, dimethylformamide, dimethyl sulfoxide, pyridine, water and the like, and a mixed solvent thereof can be mentioned.

Compound (2) is used in a proportion of generally about 1/3-5 mol per 1 mol of compound (I).

The reaction temperature is generally about 0° C. to 200° C., and the reaction time is generally about 1 hr to about 50 hr.

Where necessary, a base such as pyridine, 4-dimethylaminopyridine, triethylamine, potassium carbonate, sodium acetate, sodium hydride, sodium methoxide, lithiumdiisopropylamide and the like can be used to carry out the reaction smoothly.

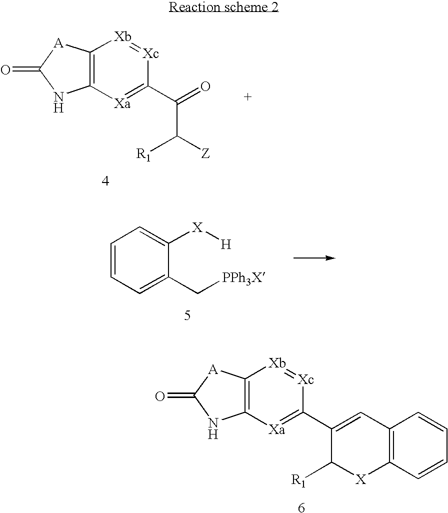

wherein X′ is a chlorine atom, a bromine atom or an iodine atom, X is O or S, and other symbols are as defined above.

Compound (6) can be produced by subjecting compound (4) and compound (5) to a fused cyclization reaction.

The fused cyclization reaction can be carried out in an inert solvent in the presence of a base.

As the solvent, for example, toluene, benzene, xylene, methanol, ethanol, propanol, isopropanol, n-butanol, acetone, chloroform, dichloromethane, 1,2-dichloroethane, tetrahydrofuran, diethyl ether, hexane, ethyl acetate, dimethylformamide, dimethyl sulfoxide, pyridine, water and the like, and a mixed solvent thereof can be mentioned.

As the base, for example, sodium methoxide, tert-butoxy potassium, pyridine, 4-dimethylaminopyridine, triethylamine, potassium carbonate, sodium acetate and the like can be mentioned.

Compound (5) is used in a proportion of generally about 1/3-5 mol per 1 mol of compound (4).

The base is used in a proportion of generally about 1/5-5 mol per 1 mol of compound (4).

The reaction temperature is generally about 0° C. to 200° C., and the reaction time is generally about 1 hr to about 50 hr.

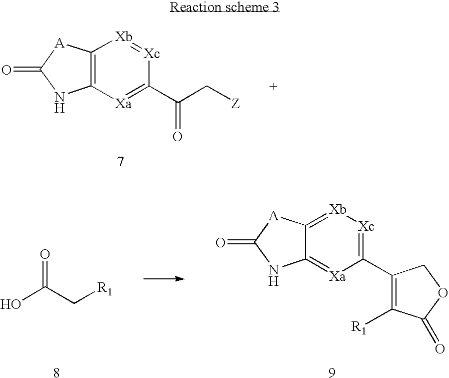

wherein each symbol is as defined above.

Compound (9) can be produced by reacting compound (7) with compound (8).

The condensation reaction can be carried out without solvent or in an inert solvent in the presence of a base.

As the solvent, for example, tetrahydrofuran, diethyl ether, dimethoxyethane, acetonitrile, hexane, toluene, benzene, dichloromethane, chloroform, 1,2-dichloroethane, ethyl acetate, methanol, ethanol, dimethylformamide, dimethyl sulfoxide, pyridine and the like, and a mixed solvent thereof can be mentioned.

As the base, for example, pyridine, 4-dimethylaminopyridine, triethylamine, DBU, potassium carbonate, cesium carbonate and the like can be mentioned.

Compound (8) is used in a proportion of generally about 1/2-2 mol per 1 mol of compound (7).

The base is used in a proportion of generally about 1/3-10 mol per 1 mol of compound (7).

The reaction temperature is generally about 0° C. to 130° C., and the reaction time is generally about 30 min to about 50 hr.

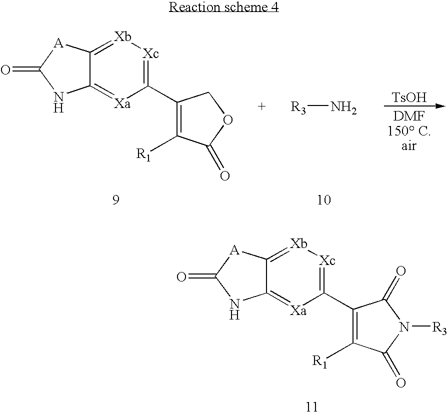

wherein each symbol is as defined above.

Compound (11) can be produced by reacting compound (9) with compound (10).

The condensation reaction can be carried out without solvent or in an inert solvent in the presence of oxygen.

As the solvent, for example, tetrahydrofuran, diethyl ether, dimethoxyethane, acetonitrile, hexane, toluene, benzene, dichloromethane, chloroform, 1,2-dichloroethane, ethyl acetate, methanol, ethanol, dimethylformamide, dimethyl sulfoxide and the like, and a mixed solvent thereof can be mentioned.

The reaction temperature is generally about 0° C. to 200° C., and the reaction time is generally about 30 min to about 50 hr.

Compound (10) is used in a proportion of generally about 1-5 mol per 1 mol of compound (9).

Where necessary, p-toluenesulfonic acid and the like can be used to carry out the reaction smoothly.

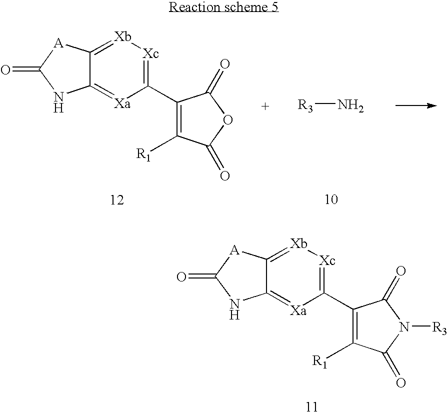

wherein each symbol is as defined above.

Compound (11) can be produced by reacting compound (12) with compound (10).

The condensation reaction can be carried out without solvent or in an inert solvent.

As the solvent, for example, tetrahydrofuran, diethyl ether, dimethoxyethane, acetonitrile, hexane, toluene, benzene, dichloromethane, chloroform, 1,2-dichloroethane, ethyl acetate, methanol, ethanol, dimethylformamide, dimethyl sulfoxide, acetic acid and the like, and a mixed solvent thereof can be mentioned.

The reaction temperature is generally about 0° C. to 200° C., and the reaction time is generally about 30 min to about 50 hr.

Compound (10) is used in a proportion of generally about 1-5 mol per 1 mol of compound (12).

Where necessary, a base such as sodium methoxide, tert-butoxy potassium, pyridine, 4-dimethylaminopyridine, triethylamine, potassium carbonate, sodium acetate and the like can be used to carry out the reaction smoothly.

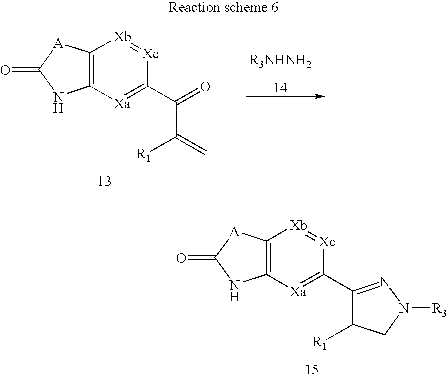

wherein each symbol is as defined above.

Compound (15) can be produced by reacting compound (13) with compound (14).

The condensation reaction can be carried out without solvent or in an inert solvent.

As the solvent, for example, tetrahydrofuran, diethyl ether, dimethoxyethane, hexane, toluene, benzene, dichloromethane, chloroform, 1,2-dichloroethane, ethyl acetate, methanol, ethanol, dimethylformamide, dimethyl sulfoxide, pyridine and the like, and a mixed solvent thereof can be mentioned.

The reaction temperature is generally about 0° C. to 200° C., and the reaction time is generally about 5 min to about 10 hr. Compound (14) is used in a proportion of generally about 1-5 mol per 1 mol of compound (13).

Where necessary, a base such as pyridine, 4-dimethylaminopyridine, triethylamine, sodium hydride, potassium carbonate, sodium hydroxide and the like can be used to carry out the reaction smoothly.

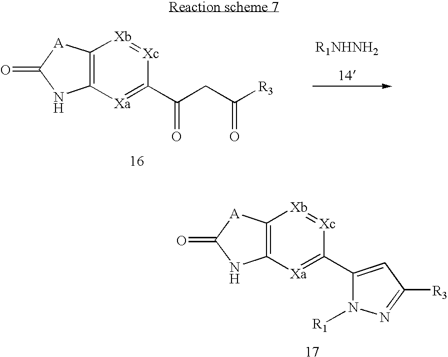

wherein each symbol is as defined above.

Compound (17) can be produced by reacting compound (16) with compound (14′).

The condensation reaction can be carried out without solvent or in an inert solvent.

As the solvent, for example, tetrahydrofuran, diethyl ether, dimethoxyethane, acetone, methanol, ethanol, hexane, toluene, benzene, dichloromethane, dimethylformamide, dimethyl sulfoxide and the like, and a mixed solvent thereof can be mentioned.

The reaction temperature is generally about 0° C. to 130° C., and the reaction time is generally about 5 min to about 50 hr.

Compound (14′) is used in a proportion of generally about 1-5 mol per 1 mol of compound (16).

Where necessary, a base such as lithium hydride, sodium hydride, sodium methoxide, sodium ethoxide, potassium t-butoxide, potassium carbonate, triethylamine and the like can be used to carry out the reaction smoothly.

Of compounds (2) used as starting materials in Reaction scheme 1, compound (21) wherein the ring moiety is imidazole can be produced by the following method.

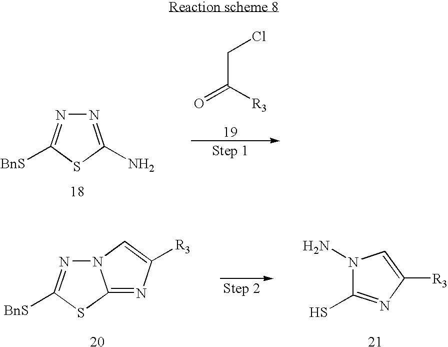

wherein each symbol is as defined above.

Compound (21) can be produced by reacting compound (18) with compound (19) to give compound (20) and subjecting compound (20) to a ring-opening reaction.

### (Step 1)

The fused cyclization reaction can be carried out without solvent or in an inert solvent.

As the solvent, for example, tetrahydrofuran, diethyl ether, dimethoxyethane, acetone, methanol, ethanol, propanol, hexane, toluene, benzene, pyridine, dichloromethane, dimethylformamide, dimethyl sulfoxide and the like, and a mixed solvent thereof can be mentioned.

Compound (19) is used in a proportion of generally about 1-5 mol per 1 mol of compound (18).

The reaction temperature is generally about 0° C. to 150° C., and the reaction time is generally about 1 hr to about 50 hr.

Where necessary, a base such as sodium hydride, sodium ethoxide, potassium carbonate, triethylamine and the like can be used to carry out the reaction smoothly.

### (Step 2)

Compound (21) can be produced by subjecting compound (20) to a ring-opening reaction.

The ring-opening reaction can be carried out by a method known per se. For example, when hydrazine is used, the reaction can be carried out without solvent or in an inert solvent.

As the solvent, for example, tetrahydrofuran, dimethoxyethane, methanol, ethanol, propanol, hexane, toluene, benzene, pyridine, dichloromethane, dimethylformamide, dimethyl sulfoxide and the like, and a mixed solvent thereof can be mentioned.

Hydrazine is used in a proportion of generally about 1-10 mol per 1 mol of compound (20).

The reaction temperature is generally about 0° C. to 150° C., and the reaction time is generally about 5 hr to about 100 hr.

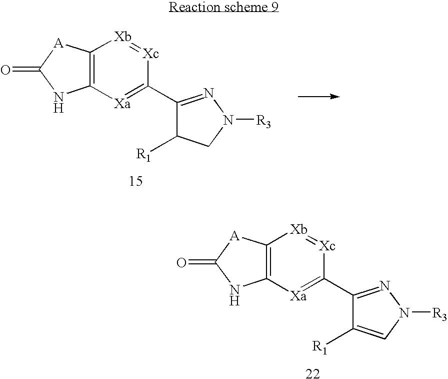

wherein each symbol is as defined above.

Compound (22) can be produced by oxidizing compound (15).

The oxidization reaction can be carried out without solvent or in an inert solvent.

As the solvent, for example, tetrahydrofuran, diethyl ether, dimethoxyethane, acetone, methanol, ethanol, propanol, hexane, toluene, benzene, pyridine, dichloromethane, dimethylformamide, dimethyl sulfoxide and the like, and a mixed solvent thereof can be mentioned.

The reaction temperature is generally about 0° C. to 200° C., and the reaction time is generally about 5 min to about 50 hr.

Where necessary, an oxidant such as 2,3-dichloro-5,6-dicyano-1,4-benzoquinone, chloranil, manganese dioxide, oxygen and the like can be used to carry out the reaction smoothly.

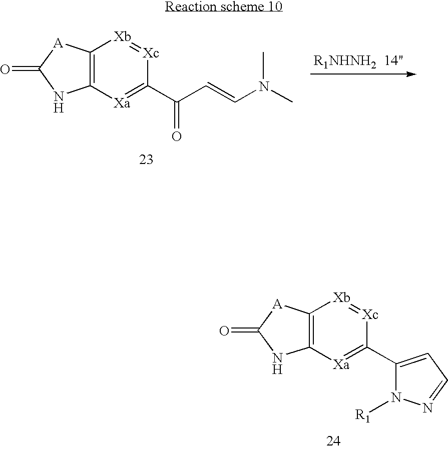

wherein each symbol is as defined above.

Compound (24) can be produced by reacting compound (23) with compound (14″).

The condensation reaction can be carried out without solvent or in an inert solvent.

As the solvent, for example, tetrahydrofuran, diethyl ether, dimethoxyethane, acetone, methanol, ethanol, hexane, toluene, benzene, dichloromethane, dimethylformamide, dimethyl sulfoxide and the like, and a mixed solvent thereof can be mentioned.

The reaction temperature is generally about 0° C. to 200° C., and the reaction time is generally about 5 min to about 50 hr.

Compound (14″) is used in a proportion of generally about 1-5 mol per 1 mol of compound (23).

Where necessary, a base such as lithium hydride, sodium hydride, sodium methoxide, sodium ethoxide, potassium t-butoxide, potassium carbonate, sodium acetate, triethylamine and the like can be used to carry out the reaction smoothly.

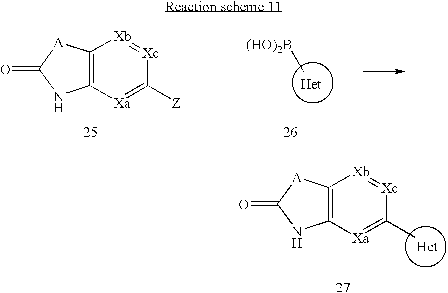

wherein each symbol is as defined above.

Compound (27) can be produced by reacting compound (25) with compound (26) in the presence of a catalyst.

The condensation reaction can be carried out without solvent or in an inert solvent.

As the solvent, for example, tetrahydrofuran, diethyl ether, dimethoxyethane, acetone, methanol, ethanol, hexane, toluene, benzene, dichloromethane, dimethylformamide, dimethyl sulfoxide, water and the like, and a mixed solvent thereof can be mentioned.

The reaction temperature is generally about 0° C. to 200° C., and the reaction time is generally about 5 min to about 50 hr.

Compound (26) is used in a proportion of generally about 0.3-5 mol per 1 mol of compound (25).

As the catalyst, for example, tetrakistriphenylphosphinepalladium, dichloro-((bis-diphenylphosphino)ferrocenyl)palladium and the like can be mentioned. The catalyst is used in a proportion of generally about 0.005-1 mol per 1 mol of compound (25).

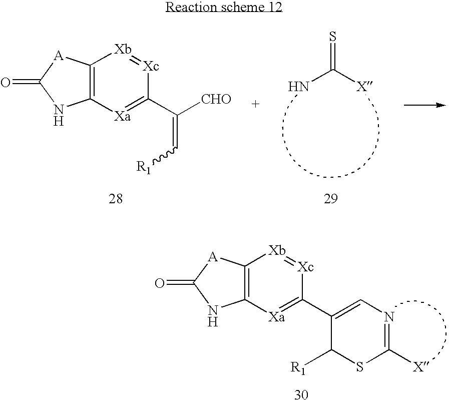

wherein X″ is a nitrogen atom, an oxygen atom or a carbon atom, and other symbols are as defined above.

Compound (30) can be produced by subjecting compound (28) and compound (29) to a fused cyclization reaction.

The fused condensation reaction can be carried out without solvent or in an inert solvent.

As the solvent, toluene, benzene, xylene, methanol, ethanol, propanol, isopropanol, n-butanol, acetone, chloroform, dichloromethane, 1,2-dichloroethane, tetrahydrofuran, 1,4-dioxane, 1,2-dimethoxyethane, diethyl ether, acetonitrile, hexane, ethyl acetate, dimethylformamide, N-methylpyrrolidone, dimethyl sulfoxide, water and the like, and a mixed solvent thereof can be mentioned.

Compound (29) is used in a proportion of generally about 1-5 mol per 1 mol of compound (28).

The reaction temperature is generally about 0° C. to 200° C., and the reaction time is generally about 1 hr to about 50 hr.

Where necessary, an acid such as hydrochloric acid, nitric acid, sulfuric acid, p-toluenesulfonic acid, acetic acid, trifluoroacetic acid and the like can be used to carry out the reaction smoothly.

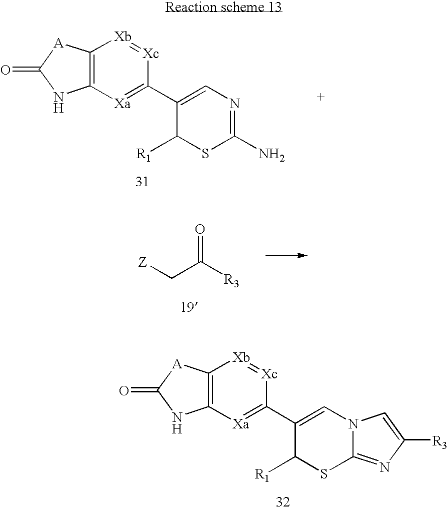

wherein each symbol is as defined above.

Compound (32) can be produced by subjecting compound (31) and compound (19′) to a fused cyclization reaction.

The fused condensation reaction can be carried out without solvent or in an inert solvent.

As the solvent, toluene, benzene, xylene, methanol, ethanol, propanol, isopropanol, n-butanol, acetone, chloroform, dichloromethane, 1,2-dichloroethane, tetrahydrofuran, 1,4-dioxane, 1,2-dimethoxyethane, diethyl ether, acetonitrile, hexane, ethyl acetate, dimethylformamide, N-methylpyrrolidone, dimethyl sulfoxide, water and the like, and a mixed solvent thereof can be mentioned.

Compound (19′) is used in a proportion of generally about 1-5 mol per 1 mol of compound (31).

The reaction temperature is generally about 0° C. to 200° C., and the reaction time is generally about 1 hr to about 50 hr.

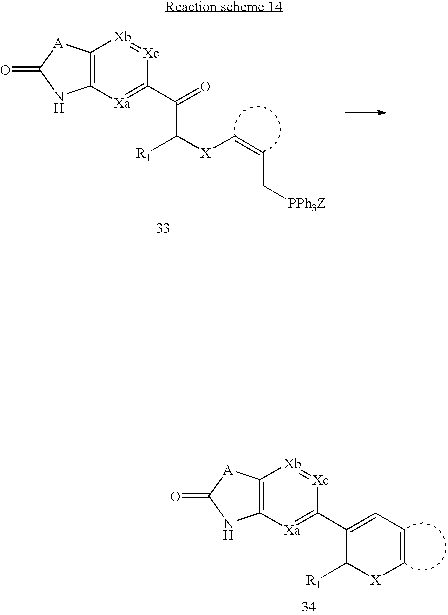

wherein the symbols are as defined above.

Compound (34) can be produced by subjecting compound (33) to a cyclization reaction.

The cyclization reaction can be carried out in an inert solvent in the presence of a base.

As the solvent, for example, toluene, benzene, xylene, methanol, ethanol, propanol, isopropanol, n-butanol, acetone, chloroform, dichloromethane, 1,2-dichloroethane, tetrahydrofuran, diethyl ether, hexane, ethyl acetate, dimethylformamide, dimethyl sulfoxide, pyridine, acetonitrile, water and the like, and a mixed solvent thereof can be mentioned.

As the base, for example, sodium methoxide, tert-butoxy potassium, n-butyllithium, pyridine, 4-dimethylaminopyridine, triethylamine, potassium carbonate, sodium acetate and the like can be mentioned.

The base is used in a proportion of generally about 1/5-5 mol per 1 mol of compound (33).

The reaction temperature is generally about 0° C. to 200° C., and the reaction time is generally about 1 hr to about 50 hr.

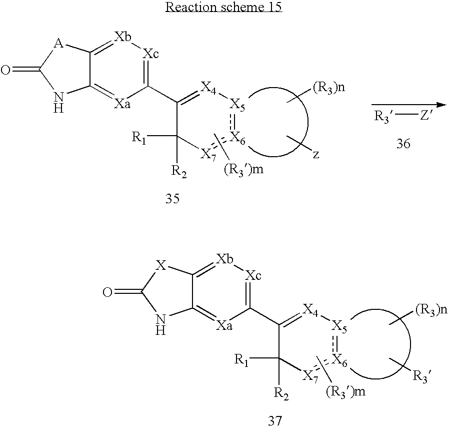

wherein Z′ is a leaving group, R₃′ is an optionally substituted amino group, an optionally substituted hydroxyl group, an optionally esterified carboxyl group, an optionally substituted aryl group, an optionally substituted chain hydrocarbon group, an optionally substituted cyclic hydrocarbon group, an optionally substituted acyl group, an optionally substituted sulfonyl group, an optionally substituted sulfinyl group, an optionally substituted mercapto group or a cyano group, and other symbols are as defined above.

Compound (37) can be produced by reacting compound (35) with compound (36).

The reaction can be carried out without solvent or in an inert solvent according to a conventional method in the presence of a metal complex having a suitable ligand and a base.

As the solvent, for example, dimethyl sulfoxide, dimethylformamide, tetrahydrofuran, toluene, benzene, xylene, chloroform, dichloromethane, 1,2-dichloroethane, diethyl ether, acetonitrile, hexane, ethyl acetate, pyridine, acetone and the like, and a mixed solvent thereof can be mentioned.

As compound (36), for example, optionally substituted alkylamines, optionally substituted alcohols, optionally substituted aryl boronic acids, optionally substituted aryl boronates, optionally substituted hydrocarbon boronic acids, optionally substituted hydrocarbon boronates, optionally substituted aryl tin compounds, optionally substituted sulfinic acids sodium salt, optionally substituted vinyl ether compounds, zinc cyanide and the like can be mentioned.

Compound (36) is used in a proportion of generally about 1-100 mol per 1 mol of compound (35).

As the metal complex having a ligand, for example, palladium, cobalt, copper and the like can be mentioned as a metal, and as the ligand, 1,1′-bis(diphenylphosphino)ferrocene, 2,2′-bis(diphenylphosphino)-1,1′-binaphthyl, triphenylphosphine, tri-tert-butylphosphine, 1, 2-bis(diphenylphosphino)ethane, 1,3-bis(diphenylphosphino)propane, proline and the like can be mentioned.

As the base, for example, potassium carbonate, cesium carbonate, potassium phosphate, sodium hydroxide, sodium tert-butoxide, triethylamine and the like can be mentioned.

The reaction temperature is generally about 0° C. to 200° C., and the reaction time is generally about 1 hr to about 50 hr.

In addition, a compound containing carbon oxide introduced therein can also be produced by carrying out this reaction under a carbon oxide atmosphere.

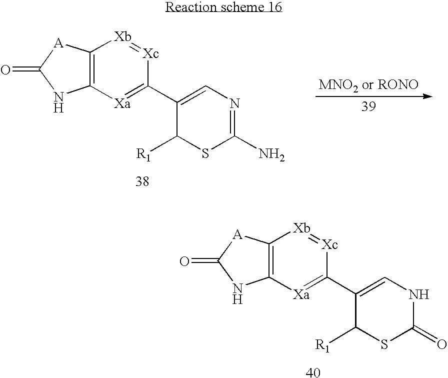

wherein M is a metal, R is an alkyl group, and other symbols are as defined above.

Compound (40) can be produced by reacting compound (38) with a nitrite salt or a nitrite ester (39).

This reaction can be carried out without solvent, or in an inert solvent.

As the nitrite salt, sodium nitrite, potassium nitrite and the like can be mentioned. As the nitrite ester, ethyl nitrite, n-butyl nitrite, iso-butyl nitrite, tert-butyl nitrite, 3-methylbutyl nitrite and the like can be mentioned.

As the solvent, for example, water, acetic acid, trifluoroacetic acid, sulfuric acid, tetrahydrofuran, diethyl ether, dimethoxyethane, acetone, methanol, ethanol, propanol, hexane, toluene, benzene, dichloromethane, dimethylformamide, dimethyl sulfoxide and the like, and a mixed solvent thereof can be mentioned.

Compound (39) is used in a proportion of generally about 1/2-10 mol per 1 mol of compound (38).

The reaction temperature is generally about 0° C. to 150° C., and the reaction time is generally about 0.5 hr to about 50 hr.

The compounds obtained in respective steps of the above-mentioned Reaction schemes can be used for the next reaction directly as the reaction mixture or as a crude product. In addition, it can also be isolated from the reaction mixture according to a conventional method, and can be easily purified by a separation means such as recrystallization, distillation, chromatography and the like.

Compound (I) may be used as a prodrug. A prodrug of compound (I) means a compound which is converted to compound (I) with a reaction due to an enzyme, an gastric acid, etc. under the physiological condition in the living body, that is, a compound which is converted to compound (I) with oxidation, reduction, hydrolysis, etc. according to an enzyme; a compound which is converted to compound (I) by hydrolysis etc. due to gastric acid, etc.

A prodrug of compound (I) may be a compound obtained by subjecting an amino group in compound (I) to an acylation, alkylation or phosphorylation (e.g., a compound obtained by subjecting an amino group in compound (I) to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation or tert-butylation, etc.); a compound obtained by subjecting a hydroxy group in compound (I) to an acylation, alkylation, phosphorylation or boration (e.g., a compound obtained by subjecting an hydroxy group in compound (I) to an acetylation, palmitoylation, propanoylation, pivaloylation, succinylation, fumarylation, alanylation or dimethylaminomethylcarbonylation); a compound obtained by subjecting a carboxyl group in compound (I) to an esterification or amidation (e.g., a compound obtained by subjecting a carboxyl group in compound (I) to an ethyl esterification, phenyl esterification, carboxymethyl esterification, dimethylaminomethyl esterification, pivaloyloxymethyl esterification, ethoxycarbonyloxyethyl esterification, phthalidyl esterification, (5-methyl-2-oxo-1,3-dioxolen-4-yl)methyl esterification, cyclohexyloxycarbonylethyl esterification or methylamidation) and the like. Any of these compounds can be produced from compound (I) by a method known per se.

A prodrug for compound (I) may also be one which is converted into compound (I) under a physiological condition, such as those described in IYAKUHIN no KAIHATSU (Development of Pharmaceuticals), Vol. 7, Design of Molecules, p. 163-198, Published by HIROKAWA SHOTEN (1990).

When compound (I) has an isomer such as optical isomer, steric isomer, positional isomer, rotational isomer and the like, any isomers and a mixture thereof are encompassed in compound (I). For example, when compound (I) has an optical isomer, an optical isomer resolved from a racemate is also encompassed in compound (I). Such isomer can be obtained as a single product by a synthesis method or a separation method (concentration, solvent extraction, column chromatography, recrystallization etc.) known per se.

Compound (I) may be a crystal, and both a single crystal and crystal mixtures are encompassed in compound (I). Crystals can be produced by crystallization according to crystallization methods known per se.

Compound (I) may be a solvate (e.g., hydrate etc.) or a non-solvate, both of which are encompassed in compound (I).

A compound labeled with an isotope (e.g., ³H, ¹⁴C, ³⁵S, ¹²⁵I and the like) and the like is also encompassed in compound (I).

The mineralocorticoid receptor antagonist of the present invention has high selectivity to steroid receptor, and selectively acts on a mineralocorticoid receptor. Therefore, it shows a weak action relating to other steroid receptors such as sex hormone action and the like, and low toxicity (e.g., more superior as a pharmaceutical agent from the aspects of acute toxicity, chronic toxicity, genetic toxicity, reproductive toxicity, cardiotoxicity, drug interaction, carcinogenicity and the like), and is useful for the prophylaxis or treatment of a disease developed or whose onset is promoted by the presence of aldosterone, or a factor induced by the presence of aldosterone, and the like in an animal, particularly mammal (e.g., human, monkey, cat, swine, horse, bovine, mouse, rat, guinea pig, dog, rabbit etc.). As such disease, systemic disease, for example, essential hypertension, primary aldosteronism, fluid accumulation type hypertension, low renin essential hypertension, malignant hypertension, renovascular hypertension, high renin hypertension, pseudo-aldosteronism, abnormal circadian variation of blood pressure, sleep apnea syndrome, cardiac failure, acute cardiac failure, chronic cardiac failure, cardiomyopathy, congestive heart failure, cardiac hypertrophy, angina pectoris, myocarditis, arrhythmia, fast pulse, cardiac infarction, asymptomatic cerebrovascular accident, transient cerebral ischemic attack, RIND, cerebral apoplexy, cerebrovascular dementia, hypertensive encephalopathy, cerebral infarction, brain edema, cerebral circulatory disturbance, recurrence and sequelae of cerebrovascular disorder (e.g., neural symptoms, mental symptoms, subjective symptoms, disorders of daily living activities etc.), ischemic peripheral circulation disorder, intermittent claudication, cardiac muscle ischemia, venous insufficiency, progress of cardiac failure after cardiac infraction, diabetic nephropathy, end stage renal failure, renal diseases (e.g., nephritis, glomerulonephritis, IgA nephropathy, progressive nephropathy, glomerulosclerosis, renal failure, thrombotic microangiopathy, complications of dialysis, organ damage including renal damage caused by irradiation etc.), arteriosclerosis including atherosclerosis (e.g., aneurysm, coronary arteriosclerosis, cerebral arteriosclerosis, peripheral arteriosclerosis etc.), vascular hypertrophy, vascular hypertrophy or occlusion and organ damage after intervention (e.g., percutaneous transluminal coronary angioplasty, stenting, coronary angioscopy, intravascular ultrasound, coronary infusion thrombolysis therapy etc.), blood vessel reocclusion or restenosis after bypass surgery, polycythemia, hypertension, organ or damage vascular hypertrophy after transplantation, rejection after transplantation, ophthalmic diseases (e.g., glaucoma, ocular hypertension disease etc.), thrombosis, multiple organ failure, endothelial dysfunction, hypertensive tinnitus, other circulatory diseases (e.g., deep-vein thrombosis, obstructive peripheral circulation disorder, obstructive arteriosclerosis, thromboangiitis obliterans, ischemic cerebral circulatory disturbance, Raynaud's disease, Buerger's disease etc.), metabolic syndrome, diabetes, diabetic complications (e.g., diabetic retinopathy, diabetic nephropathy, diabetic neuropathy etc.), metabolic or nutrient disturbance (e.g., obesity, diabetes, hyperlipidemia, hypercholesterolemia, hyperuricemia, hypokalemia, hypernatremia etc.), neurodegenerative disease (e.g., Alzheimer's disease, Parkinson's syndrome, amyotropic lateral sclerosis retinitis, AIDS encephalopathy etc.), central nerve disorders (e.g., disorder such as cerebral hemorrhage and cerebral infarction and the like and sequelae or complications thereof, head trauma, spinal injury, brain edema, disorders of sensory function, abnormality of sensory function, autonomic nervous system dysfunction, abnormality of autonomic nervous system function, multiple sclerosis etc.), dementia, memory disorders, disturbance of consciousness, amnesia, anxiety, tension, anxious mental state, mental diseases (e.g., depression, epilepsy, alcoholism etc.), inflammatory disease (e.g., arthritis such as chronic articular rheumatism, osteoarthritis, rheumatoid myelitis, periostitis and the like; inflammation after surgery or trauma; regression of puffiness; pharyngitis; cystitis; pneumonia; atopic dermatitis; inflammatory bowel disease such as Crohn's disease, ulcerative colitis and the like; meningitis; inflammatory ophthalmic diseases; inflammatory pulmonary disease such as pneumonia, silicosis, pulmonary sarcoidosis, pulmonary tuberculosis and the like), allergic disease (e.g., allergic rhinitis, conjunctivitis, gastrointestinal tract allergy, pollinosis, anaphylaxis etc.), chronic obliterative pulmonary diseases, interstitial pneumonia, carinii pneumonia, collagen disease (e.g., systemic lupus erythematosus, scleroderma, polyarteritis etc.), liver disease (e.g., hepatitis including chronic-, cirrhosis etc.), portal hypertension, gastrointestinal diseases (e.g., gastritis, gastric ulcer, gastric cancer, postgastrostomy disorder, dyspepsia, esophageal ulcer, pancreatitis, colonic polyp, cholelithiasis, hemorrhoids, variceal rupture of esophagus and stomach etc.), diseases of blood or hematopoietic organ (e.g., polycythemia, vascular purpura, autoimmune hemolytic anemia, disseminated intravascular coagulation syndrome, multiple myelopathy etc.), bone disease (e.g., bone fracture, bone refracture, osteoporosis, osteohalisteresis, Paget's disease of bone, rigid myelitis, chronic articular rheumatism, osteoarthrosis of knee and destruction of articular tissue of similar disease thereof etc.), solid tumor, tumor (e.g., malignant melanoma, malignant lymphoma, cancer of digestive organ (e.g., stomach, intestine etc.) etc.), cancer and cachexia therewith, metastasis of cancer, edema and ascites fluid associated with malignant tumor, endocrine diseases (e.g., Addison's disease, Cushing's syndrome, pheochromocytoma, primary aldosteronism etc.), Creutzfeldt-Jakob disease, diseases of urinary organ or male sex organ (e.g., cystitis, prostatomegaly, prostate cancer, sexually-transmitted diseases etc.), gynecologic diseases (e.g., climacteric disorder, gestational toxicosis, endometriosis, hysteromyoma, ovarian disease, mammary disease, sexually-transmitted diseases etc.), disease caused by environmental or occupational factor (e.g., radiation disorder, disorders caused by ultraviolet ray, infrared ray or laser beam, altitude sickness etc.), respiratory diseases (e.g., cold syndrome, pneumonia, asthma, pulmonary hypertension, pulmonary thrombosis or pulmonary embolus etc.), infections (e.g., virus infections such as cytomegalovirus, influenzavirus, herpesvirus and the like, rickettsial infections, bacterium infections etc.), toxemia (e.g., sepsis, septic shock, endotoxic shock, Gram-negative sepsis, toxic shock syndrome etc.), Otorhinolaryngological diseases (e.g., Meniere's syndrome, tinnitus, gustation disorder, dizziness, disequilibrium, dysphagia etc.), dermatic diseases (e.g., keloid, hemangioma, psoriasis etc.), dialysis hypotension, myasthenia gravis, chronic fatigue syndrome, renal edema, hepatic edema, idiopathic edema, trophedema and the like, can be mentioned. The mineralocorticoid receptor antagonist of the present invention shows a superior prophylactic or therapeutic effect on diseases for which a calcium antagonist fails to show sufficient efficacy.

As the mineralocorticoid receptor antagonist of the present invention, compound (I) or a prodrug thereof (hereinafter to be also referred to as the compound of the present invention) alone, or a pharmaceutical composition obtained by mixing with a pharmacologically acceptable carrier according to a conventional method (e.g., the method described in the Japan Pharmacopoeia etc.), such as tablets (including sugar-coated tablet, film-coated tablet), powder, granule, capsule, liquid, emulsion, suspension, injection, suppository, sustained-release preparation, plaster etc., which can be safely administered orally or parenterally (e.g., topical, rectal, intravenous administration etc.).

The content of the compound of the present invention in the pharmaceutical composition is about 0.01 to 11 wt %, preferably about 2 to 85 wt %, of the whole composition.

While the dose of the compound of the present invention varies depending on the subject of administration, administration route, disease and the like, for example, for administration of an oral preparation to an adult (body weight about 60 kg) as a therapeutic agent for cardiac failure, it is about 1 to 1000 mg, preferably about 3 to 300 mg, more preferably about 10 to 200 mg, in the amount of the compound of the present invention as an active ingredient, which can be administered once a day or in several portions a day.

The mineralocorticoid receptor antagonist of the present invention can be used in combination with a pharmaceutical agent such as an antihypertensive agent, a therapeutic agent for cardiac failure, a therapeutic agent for cardiac infarction, a therapeutic agent for diabetes, a therapeutic agent for diabetic complications, an antihyperlipidemic agent, an antiobesity agent, a diuretic agent, a chemotherapeutic agent, an immunotherapeutic agent and the like.

Examples of the antihypertensive agent include angiotensin converting enzyme inhibitors (e.g., captopril, enalapril, delapril etc.), angiotensin II antagonists (e.g., losartan, candesartan cilexetil, eprosartan, valsartan, telmisartan, irbesartan, tasosartan, olmesartan, medoxomil etc.), renin inhibitors (e.g., aliskiren etc.), calcium antagonists (e.g., manidipine, nifedipine, amlodipine, efonidipine, nicardipine, azelnidipine, cilnidipine, phelodipine etc.), β-blockers (e.g., carvedilol, propranolol, metoprolol, atenolol, carteolol etc.), α-blockers (doxazosin) and the like.

Examples of the therapeutic agents for diabetes include insulin preparations (e.g., animal insulin preparations extracted from the pancreas of bovine, swine; human insulin preparations synthesized by genetic engineering techniques using Escherichia coli or yeast, etc.), α-glucosidase inhibitor (e.g., voglibose, acarbose, miglitol, emiglitate etc.), biguanides (e.g., phenformin, metformin, buformin etc.), agents for potentiating insulin sensitivity (e.g., pioglitazone, rosiglitazone etc.), insulin secretagogues [e.g., sulfonylurea (e.g., tolbutamide, glibenclamide, gliclazide, chlorpropamide, tolazamide, acetohexamide, glyclopyramide, glimepiride, glipizide, glybuzole etc.), repaglinide, senaglinide, nateglinide, mitiglinide or calcium salt hydrate thereof, GLP-1 etc.], amylin agonist (e.g., pramlintide etc.), phosphotyrosine phosphatase inhibitor (e.g., vanadic acid etc.) and the like.

Examples of the therapeutic agent for diabetic complications include aldose reductase inhibitors (e.g., tolrestat, epalrestat, zenarestat, zopolrestat, minalrestat, fidarestat, SNK-860, CT-112 etc.), neurotrophic factors (e.g., NGF, NT-3, BDNF etc.), neurotrophic factor-production promoter, PKC inhibitors (e.g., LY-333531 etc.), AGE inhibitors (e.g., ALT946, pimagedine, pyratoxanthine, N-phenacylthiazolium bromide (ALT766), EXO-226 etc.), active oxygen scavengers (e.g., thioctic acid etc.), cerebral vasodilators (e.g., tiapuride, mexiletine etc.) and the like.

Examples of the antihyperlipidemia agent include statin compounds which are cholesterol synthesis inhibitors (e.g., pravastatin, simvastatin, lovastatin, atorvastatin, fluvastatin, cerivastatin, itavastatin or a salt thereof (e.g., sodium salt etc.) etc.), squalene synthase inhibitor or fibrate compounds having a triglyceride lowering action (e.g., bezafibrate, clofibrate, simfibrate, clinofibrate etc.) and the like.

Examples of the antiobesity agents include antiobesity agents acting on the central nervous system (e.g., dexfenfluramine, fenfluramine, phentermine, sibutramine, amfepramone, dexamphetamine, mazindol, phenylpropanolamine, clobenzorex, rimonabant etc.), pancreatic lipase inhibitors (e.g., orlistat etc.), β3 agonists (e.g., CL-316243, SR-58611-A, UL-TG-307, SB-226552, AJ-9677, BMS-196085, AZ-40140 etc.), peptidic anorexiants (e.g., leptin, CNTF (Ciliary Neurotropic Factor) etc.), cholecystokinin agonists (e.g., lintitript, FPL-15849 etc.) and the like.

Examples of the diuretic agent include, for example, xanthine derivatives (e.g., theobromine sodium salicylate, theobromine calcium salicylate etc.), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide etc.), carbonate dehydratase inhibitors (e.g., acetazolamide etc.), chlorobenzenesulfonamide preparations (e.g., chlorthalidone, mefruside, indapamide etc.), azosemide, isosorbide, ethacrynic acid, piretanide, bumetanide, furosemide, torasemide and the like.

Examples of the chemotherapeutic agent include alkylating agents (e.g., cyclophosphamide, ifosfamide etc.), metabolic antagonists (e.g., methotrexate, 5-fluorouracil etc.), antitumor antibiotics (e.g., mitomycin, adriamycin etc.), plant-derived antitumor agents (e.g., vincristine, vindesine, Taxol etc.), cisplatin, carboplatin, etoposide and the like. Particularly, 5-fluorouracil derivatives (e.g., Furtulon, Neo-Furtulon and the like) are preferable.

Examples of the immunotherapeutic agent include microorganism or bacterium-derived components (e.g., muramyl dipeptide derivative, Picibanil etc.), polysaccharides having an immunity enhancing activity (e.g., lentinan, schizophyllan, krestin etc.), cytokine obtained by genetic engineering (e.g., interferon, interleukin (IL) etc.), colony stimulating agents (e.g., granulocyte colony stimulating factor, erythropoietin etc.) and the like. Particularly, IL-1, IL-2, IL-12 and the like are preferable. Moreover, pharmaceutical agents whose cachexia-improving effect is observed in animal models or clinically, that is, cyclooxygenase inhibitors (e.g., indomethacin etc.) (Cancer Research, vol. 49, p. 5935-5939, 1989), progesterone derivatives (e.g., megestrol acetate etc.) (Journal of Clinical Oncology, vol. 12, p. 213-225, 1994), glucocorticoids (e.g., dexamethasone etc.), metoclopramide pharmaceuticals, tetrahydrocannabinol pharmaceuticals (same as those mentioned above), fat metabolism ameliorating agents (e.g., eicosapentanoic acid etc.) (British Journal of Cancer, vol. 68, p. 314-318, 1993), growth hormone, IGF-1, or antibodies to TNF-α, LIF, IL-6 or oncostatin M, which are cachexia-inducing factors, and the like can also be used in combination with the pharmaceutical agent of the present invention.

Moreover, pharmaceutical agents generally used for the treatment of cardiac failure, such as digitalis, catecholamine (e.g., dobutamin, dopamine, denopamine, zamoterol etc.), nitrate drugs (e.g., nitroglycerol etc.), hydralazine, PDE inhibitors (e.g., milrinone etc.), Ca sensitivity increasing agents (e.g., pimobendan etc.), thrombolytic agents (e.g., t-PA etc.), anticoagulants (e.g., heparin, warfarin etc.), anti-platelet agents (e.g., aspirin etc.), antiarrhythmic agents (e.g., amiodarone etc.), α-blockers (e.g., prazosin etc.), atrial diuretic peptide, NEP inhibitors (e.g., fasidotril etc.), endothelin antagonists (e.g., bosentan etc.), vasopressin antagonists (e.g., conivaptan etc.), matrix metalloprotease inhibitors

and the like can be mentioned.

The mineralocorticoid receptor antagonist of the present invention can also be used in combination with biological preparations (e.g., antibody, vaccine preparation etc.) when applying to the above-mentioned disease. In addition, it can also be applied for a combination therapy in combination with a gene therapy and the like. As the antibody and vaccine preparation, for example, vaccine preparations for angiotensin II, vaccine preparation for CETP, CETP antibody, TNF α-antibody, antibody to other cytokine, amyloid β vaccine preparation, diabetes type 1 vaccine (e.g., DIAPEP-277 of Peptor etc.) and the like, antibody to or vaccine preparation for cytokine, renin angiotensin enzyme and products thereof, antibody to or vaccine preparation for enzyme and protein involved in blood lipid metabolism, antibody to or vaccine relating to enzyme and protein involved in blood coagulation or fibrinolytic system, antibody to or vaccine preparation for protein involved in sugar metabolism and insulin resistance and the like can be mentioned. In addition, as methods for the gene therapy, for example, a treatment method using a gene relating to cytokine, rennin or angiotensin enzyme and a product thereof, a treatment method using a gene relating to the signal transduction system such as β receptor, adenyl cyclase and the like, a treatment method using a gene relating to GRK such as β ARKct, β arrestin and the like, a treatment method using a DNA decoy such as NFκB decoy and the like, a treatment method using antisense, a treatment method using a gene (e.g., gene relating to metabolism, excretion or absorption of cholesterol, triglyceride, HDL-cholesterol or blood phospholipid etc.) relating to enzyme or protein involved in blood lipid metabolism, a treatment method using a gene relating to enzyme or protein (e.g., growth factor such as HGF, VEGF and the like) involved in angiogenesis therapy for peripheral vessel obstruction and the like, a treatment method using a gene relating to protein involved in sugar metabolism or insulin resistance, antisense to cytokine such as TNF and the like, and the like can be mentioned. In addition, various organ regeneration methods such as cardiac regeneration, kidney regeneration, pancreas regeneration, revascularization and the like, a blood vessel and cardiac muscle neogenesis therapy utilizing transplantation of bone-marrow cell (e.g., myelomonocytic cells, myeloid stem cell), endothelial progenitor cells and other cells having a differentiation potential to muscle (e.g., embryonic stem cell, hematopoietic stem cell, myeloid stem cell, myoblast etc.) may be used in combination. When the agent of the present invention is used in combination with a combination drug, the agent of the present invention and the combination drug may be administered as separate pharmaceutical agents, or may be administered as a single pharmaceutical agent. For combined use as separate pharmaceutical agents, the time of administration of the agent of the present invention and that of the combination drug are not limited, and they may be administered simultaneously or in a staggered manner to the administration subject. Moreover, two or more kinds of combination drugs may be used in combination at an appropriate ratio.

The dose of the combination drug can be appropriately determined based on the dose of each drug employed clinically. In addition, the administration ratio of the agent of the present invention and the combination drug can be appropriately determined according to the administration subject, administration route, target disease, condition, combination, and the like.

The mineralocorticoid receptor antagonist of the present invention has a superior mineralocorticoid receptor antagonistic action, and is advantageously used for the prophylaxis or treatment of circulatory diseases such as hypertension, cardiac failure and the like.

### EXAMPLES

In the following Preparations and Examples, melting point, mass spectrum (MS) and nuclear magnetic resonance spectrum (NMR) were measured under the following conditions. melting point measurement tools: Yanagimoto micromelting point measuring apparatus, or Büchi melting point measuring apparatus type B-545 was used.

MS measurement tools: Waters Corporation ZMD, Waters Corporation ZQ2000 or Micromass Ltd., platform II, ionization method: Electron Spray Ionization (ESI) or Atmospheric Pressure Chemical Ionization (APCI). Unless specifically indicated, ESI was used.

NMR measurement tools: Varian Inc. Varian Gemini 200 (200 MHz), Varian Mercury-300 (300 MHz), Varian INOVA-400 (400 MHz) or Bruker BioSpin Corp. AVANCE 300. Chemical shifts are given in ppm with tetramethylsilane as the internal standard, and coupling constants (J) are given in hertz (Hz).

In Preparations and Examples, purification by preparative HPLC was performed under the following conditions.

Preparative HPLC tools: Waters Corporation, UV purification system
column: Develosil ODS-UG-10
solvent: Solution A; 0.1% trifluoroacetic acid-containing water
Solution B; 0.1% trifluoroacetic acid-containing acetonitrile
gradient: 10 min gradient, 5-100% gradient
Gradient cycle: 0.00 min (A/B=95/5), 1.00 min (A/B=95/5), 2.00 min (A/B=80/20), 5.00 min (A/B=5/95), 5.10 min (A/B=0/100), 7.00 min (A/B=100/0)
flow rate: 150 mL/min, detection method: UV 220 nm

The abbreviations in Reference Examples and Examples follow those generally used in the pertinent technical field and, for example, mean the following.

s: singlet

d: doublet

t: triplet

q: quartet

dd: double doublet

dt: double triplet

dq: double quartet

ddd: double double doublet

td: triple doublet

tt: triple triplet

m: multiplet

br: broad

brs: broad singlet

J: coupling constant

WSC: water-soluble carbodiimide

THF: tetrahydrofuran

DMF: dimethylformamide

DMSO: dimethyl sulfoxide

DBU: 1,8-diazabicyclo[5.4.0]undeca-7-en

EDCI: 1-ethyl-3-(3-dimethylaminopropyl)carbodiimide

HOBt: 1-hydroxybenzotriazole

IPE: diisopropyl ether

DMAP: 4-(dimethylamino)pyridine

DCM: dichloromethane

DCE: dichloroethane

IPA: isopropyl alcohol

TFA: trifluoroacetic acid

TEA: triethylamine

RP-HPLC: reverse phase high performance liquid chromatography

EtOAc: ethyl acetate

NBS: N-bromosuccinimide

NIS: N-iodosuccinimide

dppf: 1,1′-bis(diphenylphosphino)ferrocene

Pd₂dba₃: (tris(dibenzylideneacetone)dipalladium(0)

NCS: N-chlorosuccinimide

### Preparation 1

### 6-Isobutyryl-2H-1,4-benzoxazin-3(4H)-one

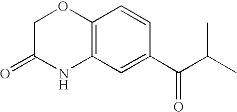

To a suspension of 2H-1,4-benzoxazin-3(4H)-one (10.0 g) in dichloroethane (120 ml) was added portionwise aluminum trichloride (20.0 g) in a water bath. Then isobutyryl chloride (8.4 ml) was added dropwise, and the mixture was stirred at room temperature for 12 hr and at 40° C. for 3 hr, cooled, and then poured into ice-water (200 ml). The resulting crystals were collected by filtration and washed with H₂O and then with dichloromethane. The organic layer of the filtrate was separated, dried over MgSO₄ and concentrated in vacuo. Residual crystals were washed with diisopropyl ether. Crystals were combined to give the title compound as colorless crystals (10.9 g).

¹H-NMR (300 MHz, CDCl₃) δ: 1.21 (6H, d, J=6.8 Hz), 3.49 (1H, sept, J=6.8 Hz), 4.70 (2H, s), 7.03 (1H, d, J=8.4 Hz), 7.50 (1H, d, J=2.2 Hz), 7.61 (1H, dd, J=8.4, 2.2 Hz), 8.16 (1H, br).

MS (ESI) 220 (M+1).

### Preparation 2

### 6-(2-Bromo-2-methylpropanoyl)-2H-1,4-benzoxazin-3(4H)-one

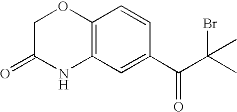

To a suspension of 6-isobutyryl-2H-1,4-benzoxazin-3(4H)-one (10.0 g) in acetic acid (100 ml) was added 25% hydrogen bromide in acetic acid (25 ml). Then pyridinium hydrobromide perbromide (15.32 g) was added portionwise. The mixture was stirred at room temperature for 2 hr and concentrated, and the residue was treated with ethyl acetate and water. The organic layer was separated, and the aqueous layer was further extracted with ethyl acetate. The organic layers were combined, dried over MgSO₄ and concentrated in vacuo. The solid was washed with H₂O and hexane and then dried to give the title compound as colorless crystals (12.0 g).

¹H-NMR (300 MHz, CDCl₃) δ: 2.03 (6H, s), 4.70 (2H, s), 7.00 (1H, d, J=8.7 Hz), 7.64 (1H, d, J=1.8 Hz), 7.96 (1H, dd, J=8.7, 2.4 Hz), 8.10 (1H, br).

MS (ESI) 298 (M+1).

### Example 1

### 6-(7,7-Dimethyl-7H-[1,2,4]triazolo[3,4-b][1,3,4]thiadiazin-6-yl)-2H-1,4-benzoxazin-3(4H)-one

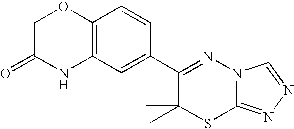

A suspension of 6-(2-bromo-2-methylpropanoyl)-2H-1,4-benzoxazin-3(4H)-one (1.0 g) and 4-amino-3-mercapto-4H-1,2,4-triazole (0.41 g) in ethanol (20 ml) and toluene (10 ml) was heated under reflux for 24 hr. The solvent was removed and then the residue was treated with ethyl acetate and saturated NaHCO₃. The organic layer was separated, dried over MgSO₄ and concentrated in vacuo. The residue was crystallized from ethyl acetate/methanol to give the title compound (370 mg).

¹H-NMR (300 MHz, CDCl₃) δ: 1.63 (6H, s), 4.70 (2H, s), 7.05 (1H, s), 7.08 (2H, m), 8.63 (1H, s), 9.35 (1H, br).

MS (ESI) 316 (M+1).

### Example 2

### 6-(3,7,7-Trimethyl-7H-[1,2,4]triazolo[3,4-b][1,3,4]thiadiazin-6-yl)-2H-1,4-benzoxazin-3(4H)-one

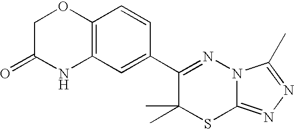

A suspension of 6-(2-bromo-2-methylpropanoyl)-2H-1,4-benzoxazin-3(4H)-one (1.0 g) and 4-amino-5-methyl-4H-1,2,4-triazole-3-thiol (0.46 g) in ethanol (20 ml) and toluene (10 ml) was heated under reflux for 24 hr. The solvent was removed and then the residue was treated with ethyl acetate and saturated NaHCO₃. The organic layer was separated, dried over MgSO₄ and concentrated in vacuo. The residue was crystallized from methanol to give the title compound (0.40 g).

mp. 249-250° C.

¹H-NMR (300 MHz, CDCl₃) δ: 1.61 (6H, s), 2.56 (3H, s), 4.69 (2H, s), 6.99 (1H, m), 7.07 (2H, s), 8.66 (1H, br).

### Preparation 3

### 2-(Benzylthio)imidazo[2,1-b][1,3,4]thiadiazole

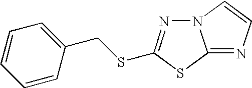

A mixture of 5-(benzylthio)-1,3,4-thiadiazol-2-amine (5.0 g) and 45% chloroacetaldehyde (3.9 g) in ethanol (20 mL) and toluene (10 ml) was refluxed for 12 hr. The solvent was removed in vacuo and then the residue was treated with ethyl acetate and saturated NaHCO₃. The organic layer was separated, dried over MgSO₄ and concentrated in vacuo. The residue was chromatographed on silica gel with hexane/ethyl acetate as an eluent to give the title compound (1.27 g).

¹H-NMR (300 MHz, CDCl₃) δ: 4.44 (2H, s), 7.30-7.40 (6H, m), 7.68 (1H, m).

### Preparation 4

### 2-(Benzylthio)-6-methylimidazo[2,1-b][1,3,4]thiadiazole

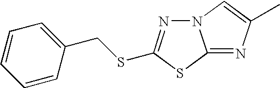

The title compound (3.00 g) was obtained from 5-(benzylthio)-1,3,4-thiadiazol-2-amine (10.0 g) and chloroacetone (3.9 ml) according to a method similar to the procedure for 2-(benzylthio)imidazo[2,1-b][1,3,4]thiadiazole.

¹H-NMR (300 MHz, CDCl₃) δ: 2.34 (3H, s), 4.40 (2H, s), 7.29-7.43 (5H, m), 7.34 (1H, s).

### Preparation 5

### 2-(Benzylthio)-6-(trifluoromethyl)imidazo[2,1-b][1,3,4]thiadiazole

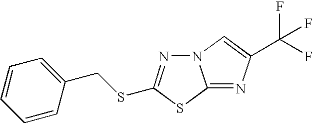

The title compound (0.80 g) was obtained from 5-(benzylthio)-1,3,4-thiadiazol-2-amine (6.0 g) and 3-bromo-1,1,1-trifluoroacetone (4.2 ml) according to a method similar to the procedure for 2-(benzylthio)imidazo[2,1-b][1,3,4]thiadiazole.

¹H-NMR (300 MHz, DMSO-d₆) δ: 4.60 (2H, s), 7.29-7.37 (3H, m), 7.44-7.47 (2H, m), 8.86 (1H, m).

MS (ESI) 316 (M+1).

### Preparation 6

### 2-(Benzylthio)-6-propylimidazo[2,1-b][1,3,4]thiadiazole

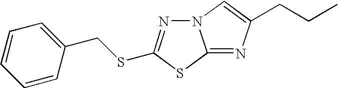

The title compound (4.2 g) was obtained from 5-(benzylthio)-1,3,4-thiadiazol-2-amine (8.4 g) and 1-chloropentan-2-one (6.0 g) according to a method similar to the procedure for 2-(benzylthio)imidazo[2,1-b][1,3,4]thiadiazole.

¹H-NMR (300 MHz, DMSO-d₆) δ: 0.90 (3H, t, J=7.3 Hz), 1.57-1.68 (2H, m), 2.94-2.57 (2H, m), 4.52 (2H, s), 7.30-7.46 (5H, m), 7.85 (1H, s). MS (ESI) 290 (M+1).

### Preparation 7

### 2-(Benzylthio)-6-(ethoxymethyl)imidazo[2,1-b][1,3,4]thiadiazole

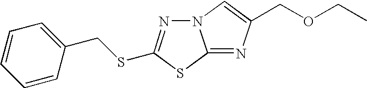

The title compound (1.2 g) was obtained from 5-(benzylthio)-1,3,4-thiadiazol-2-amine (10.4 g) and 1-ethoxy-3-chloroacetone (7.0 g) according to a method similar to the procedure for 2-(benzylthio)imidazo[2,1-b][1,3,4]thiadiazole.

¹H-NMR (300 MHz, DMSO-d₆) δ: 1.11 (3H, t, J=7.0 Hz), 3.48 (2H, q, J=7.0 Hz), 4.38 (2H, s), 4.55 (2H, s), 7.28-7.37 (3H, m), 7.43-7.46 (2H, m), 8.07 (1H, s).

MS (ESI) 306 (M+1).

### Preparation 8

### 1-Amino-1H-imidazole-2-thiol

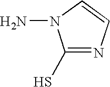

A solution of 2-(benzylthio)imidazo[2,1-b][1,3,4]thiadiazole (1.2 g) and hydrazine monohydrate (2.4 g) in ethanol (20 mL) was stirred under reflux for 50 hr. The solvent was removed in vacuo and then the residue was chromatographed on silica gel with hexane/ethyl acetate as an eluent to give the title compound (0.19 g).

¹H-NMR (300 MHz, DMSO-d₆) δ: 5.62 (2H, s), 6.80 (1H, m), 7.04 (1H, m), 12.06 (1H, br).

### Preparation 9

### 1-Amino-4-methyl-1H-imidazole-2-thiol

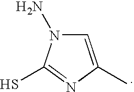

The title compound (0.42 g) was obtained from 2-(benzylthio)-6-methylimidazo[2,1-b][1,3,4]thiadiazole (3.0 g) and hydrazine monohydrate (5.7 g) according to a method similar to the procedure for 1-amino-1H-imidazole-2-thiol.

¹H-NMR (300 MHz, CDCl₃) δ: 1.99 (3H, s), 5.53 (2H, s), 6.72 (1H, s), 11.80 (1H, br).

### Preparation 10

### 1-Amino-4-(trifluoromethyl)-1H-imidazole-2-thiol

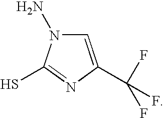

The title compound (0.17 g) was obtained from 2-(benzylthio)-6-(trifluoromethyl)imidazo[2,1-b][1,3,4]thiadiazole (0.80 g) and hydrazine monohydrate (1.3 g) according to a method similar to the procedure for 1-amino-1H-imidazole-2-thiol.

¹H-NMR (300 MHz, DMSO-d₆) δ: 5.76 (2H, s), 7.87 (1H, s), 13.5 (1H, br).

### Preparation 11

### 1-Amino-4-propyl-1H-imidazole-2-thiol

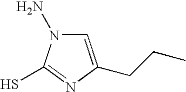

The title compound (0.95 g) was obtained from 2-(benzylthio)-6-propylimidazo[2,1-b][1,3,4]thiadiazole (4.0 g) and hydrazine monohydrate (6.9 g) according to a method similar to the procedure for 1-amino-1H-imidazole-2-thiol.

¹H-NMR (300 MHz, DMSO-d₆) δ: 0.86 (3H, t, J=7.5 Hz), 1.52 (2H, sept, J=7.5 Hz), 2.31 (2H, t, J=7.5 Hz), 5.55 (2H, s), 6.76 (1H, s), 11.96 (1H, br).

### Preparation 12

### 1-Amino-4-(ethoxymethyl)-1H-imidazole-2-thiol

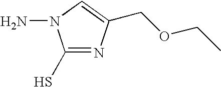

The title compound (0.35 g) was obtained from 2-(benzylthio)-6-(ethoxymethyl)imidazo[2,1-b][1,3,4]thiadiazole (1.2 g) and hydrazine monohydrate (2.0 g) according to a method similar to the procedure for 1-amino-1H-imidazole-2-thiol.

¹H-NMR (300 MHz, DMSO-d₆) δ: 1.09 (3H, t, J=7.0 Hz), 3.39 (2H, q, J=7.0 Hz), 4.17 (2H, s), 5.61 (2H, s), 7.04 (1H, s), 12.23 (1H, br).

### Example 3

### 6-(7-Phenyl-7H-[1,2,4]triazolo[3,4-b][1,3,4]thiadiazin-6-yl)-2H-1,4-benzoxazin-3(4H)-one

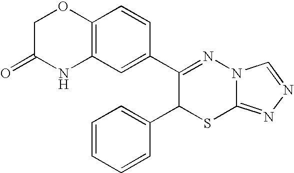

A suspension of 6-[bromo(phenyl)acetyl]-2H-1,4-benzoxazin-3(4H)-one (1.0 g) and 4-amino-4H-1,2,4-triazole-3-thiol (0.28 g) in ethanol (20 mL) and toluene (10 mL) was stirred under reflux for 24 hr. The solvent was removed and then the residue was treated with ethyl acetate and aqueous NaHCO₃. The organic layer was separated, dried over MgSO₄ and concentrated in vacuo. The residue was purified by crystallization from ethyl acetate/methanol to give the title compound (0.33 g).

¹H-NMR (300 MHz, CDCl₃) δ: 4.69 (2H, s), 5.48 (1H, s), 7.02 (1H, m), 7.13-7.16 (2H, m), 7.26-7.28 (3H, m), 7.41 (1H, m), 7.52 (1H, s), 8.67 (1H, s), 8.75 (1H, br).

MS (ESI) 364 (M+1).

### Example 4

### 6-(3-Methyl-7-phenyl-7H-[1,2,4]triazolo[3,4-b][1,3,4]thiadiazin-6-yl)-2H-1,4-benzoxazin-3(4H)-one

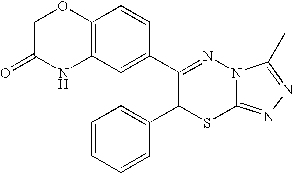

A suspension of 6-[bromo(phenyl)acetyl]-2H-1,4-benzoxazin-3(4H)-one (1.0 g) and 4-amino-5-methyl-4H-1,2,4-triazole-3-thiol (0.31 g) in ethanol (20 mL) and toluene (10 mL) was stirred under reflux for 24 hr. The solvent was removed and then the residue was treated with ethyl acetate and aqueous NaHCO₃. The organic layer was separated, dried over MgSO₄ and concentrated in vacuo. The residue was purified by crystallization from THF/methanol to give the title compound (0.06 g).

¹H-NMR (300 MHz, DMSO-d₆) δ: 2.59 (3H, s), 4.66 (2H, s), 6.28 (1H, s), 7.07 (1H, m), 7.13-7.18 (2H, m), 7.30-7.33 (3H, m), 7.50 (1H, m), 7.63 (1H, s), 10.9 (1H, br).

MS (ESI) 378 (M+1).

### Example 5

### 6-(2-Phenyl-2H-imidazo[2,1-b][1,3,4]thiadiazin-3-yl)-2H-1,4-benzoxazin-3(4H)-one

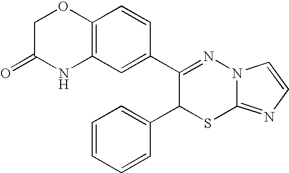

A solution of 6-[bromo(phenyl)acetyl]-2H-1,4-benzoxazin-3(4H)-one (0.5.7 g) and 1-amino-1H-imidazole-2-thiol (0.19 g) in ethanol (20 mL) and toluene (10 mL) was stirred under reflux for 12 hr. The solvent was removed and then the residue was treated with ethyl acetate and aqueous NaHCO₃. The organic layer was separated, dried over MgSO₄ and concentrated in vacuo. The residue was chromatographed on silica gel with ethyl acetate/hexane as an eluent to give the title compound (0.10 g).

¹H-NMR (300 MHz, DMSO-d₆) δ: 4.65 (2H, s), 6.14 (1H, s), 6.99-7.00 (1H, m), 7.04-7.07 (1H, m), 7.13-7.16 (2H, m), 7.27-7.34 (3H, m), 7.41-7.45 (1H, m), 7.57-7.58 (1H, m), 7.78 (1H, m), 10.92 (1H, br).

MS (ESI) 363 (M+1).

### Example 6

### 6-(7-Methyl-2-phenyl-2H-imidazo[2,1-b][1,3,4]thiadiazin-3-yl)-2H-1,4-benzoxazin-3(4H)-one

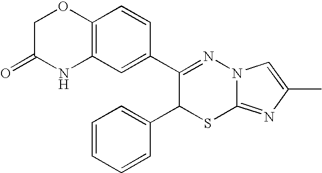

The title compound (0.25 g) was obtained from 6-[bromo(phenyl)acetyl]-2H-1,4-benzoxazin-3(4H)-one (0.50 g) and 1-amino-4-methyl-1H-imidazole-2-thiol (0.20 g) according to a method similar to the procedure for 6-(2-phenyl-2H-imidazo[2,1-b][1,3,4]thiadiazin-3-yl)-2H-1,4-benzoxazin-3(4H)-one.

¹H-NMR (300 MHz, DMSO-d₆) δ: 2.06 (3H, s), 4.63 (2H, s), 6.06 (1H, s), 7.01-7.04 (1H, m), 7.12-7.15 (2H, m), 7.25-7.30 (3H, m), 7.37-7.40 (1H, m), 7.45 (1H, s), 7.53-7.54 (1H, m), 10.9 (1H, br).

MS (ESI) 377 (M+1).

### Example 7

### 6-[2-Phenyl-7-(trifluoromethyl)-2H-imidazo[2,1-b][1,3,4]thiadiazin-3-yl]-2H-1,4-benzoxazin-3(4H)-one

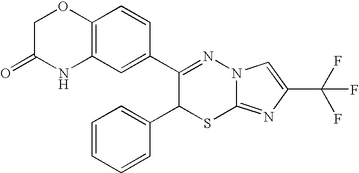

The title compound (0.09 g) was obtained from 6-[bromo(phenyl)acetyl]-2H-1,4-benzoxazin-3(4H)-one (0.32 g) and 1-amino-4-(trifluoromethyl)-1H-imidazole-2-thiol (0.17 g) according to a method similar to the procedure for 6-(2-phenyl-2H-imidazo[2,1-b][1,3,4]thiadiazin-3-yl)-2H-1,4-benzoxazin-3(4H)-one.

¹H-NMR (300 MHz, DMSO-d₆) δ: 4.67 (2H, s), 6.28 (1H, s), 7.07-7.10 (1H, m), 7.16-7.19 (2H, m), 7.30-7.37 (3H, m), 7.44-7.47 (1H, m), 7.56-7.57 (1H, m), 8.55-8.56 (1H, m), 10.97 (1H, br).

MS (ESI) 431 (M+1).

### Example 8

### 6-(2-Phenyl-7-propyl-2H-imidazo[2,1-b][1,3,4]thiadiazin-3-yl)-2H-1,4-benzoxazin-3(4H)-one

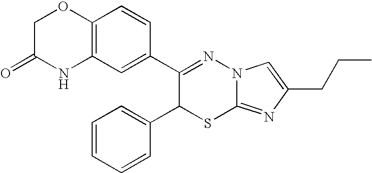

The title compound (0.28 g) was obtained from 6-[bromo(phenyl)acetyl]-2H-1,4-benzoxazin-3(4H)-one (0.50 g) and 1-amino-4-propyl-1H-imidazole-2-thiol (0.23 g) according to a method similar to the procedure for 6-(2-phenyl-2H-imidazo[2,1-b][1,3,4]thiadiazin-3-yl)-2H-1,4-benzoxazin-3(4H)-one.

¹H-NMR (300 MHz, DMSO-d₆) δ: 0.87 (3H, t, J=7.5 Hz), 1.56 (2H, sept, J=7.5 Hz), 2.39 (2H, t, J=7.5 Hz), 4.64 (2H, s), 6.08 (1H, s), 7.03-7.05 (1H, m), 7.13-7.15 (2H, m), 7.27-7.33 (3H, m), 7.38-7.42 (1H, m), 7.48 (1H, s), 7.54-7.55 (m, 1H), 10.9 (1H, br).

MS (ESI) 405 (M+1).

### Example 9

### 6-[7-(Ethoxymethyl)-2-phenyl-2H-imidazo[2,1-b][1,3,4]thiadiazin-3-yl]-2H-1,4-benzoxazin-3(4H)-one

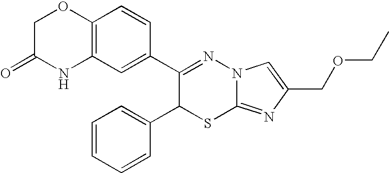

The title compound (0.09 g) was obtained from 6-[bromo(phenyl)acetyl]-2H-1,4-benzoxazin-3(4H)-one (0.40 g) and 1-amino-4-(ethoxymethyl)-1H-imidazole-2-thiol (0.20 g) according to a method similar to the procedure for 6-(2-phenyl-2H-imidazo[2,1-b][1,3,4]thiadiazin-3-yl)-2H-1,4-benzoxazin-3(4H)-one.

¹H-NMR (300 MHz, DMSO-d₆) δ: 1.09 (3H, t, J=7.0 Hz), 3.44 (2H, q, J=7.0 Hz), 4.24 (2H, s), 4.64 (2H, s), 6.12 (1H, s), 7.03-7.06 (1H, m), 7.14-7.17 (2H, m), 7.27-7.34 (3H, m), 7.40-7.43 (1H, m), 7.55-7.56 (1H, m), 7.71 (1H, s), 10.9 (1H, br).

MS (ESI) 421 (M+1).

### Example 10

### 6-(7-Methoxy-7-phenyl-7H-[1,2,4]triazolo[3,4-b][1,3,4]thiadiazin-6-yl)-2H-1,4-benzoxazin-3(4H)-one

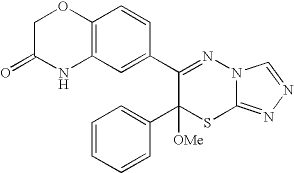

To a solution of 6-(2-phenyl-2H-triazolo[2,1-b][1,3,4]thiadiazin-3-yl)-2H-1,4-benzoxazin-3(4H)-one (0.18 g) in MeOH (10 mL) was added dropwise a solution of 3-chloroperbenzoic acid (0.12 g) in MeOH (2 mL) at 0° C. in an ice-bath. The reaction mixture was slowly warmed up to room temperature and stirred for 3 days. The solvent was removed in vacuo and the residue was treated with THF, aqueous Na₂CO₃ and aqueous NaHCO₃. The organic layer was separated, dried over MgSO₄ and concentrated. The residue was chromatographed on silica gel with hexane/ethyl acetate as an eluent and followed by recrystallization from ethyl acetate/hexane to give the title compound as crystals (0.07 g).

¹H-NMR (300 MHz, DMSO-d₆) δ: 3.33 (3H, s), 4.58 (2H, s), 6.82-6.85 (1H, m), 7.04-7.08 (1H, m), 7.20 (1H, m), 7.31-7.38 (3H, m), 7.48-7.52 (2H, m), 9.34 (1H, s), 10.8 (1H, br).

MS (ESI) 394 (M+1).

### Example 11

### 6-[4-(2,4-Dichlorophenyl)-5-oxo-2,5-dihydrofuran-3-yl]-2H-1,4-benzoxazin-3(4H)-one

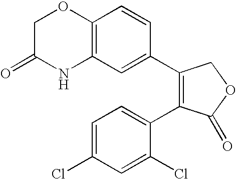

A suspension of 2,4-dichlorophenylacetic acid (2.0 g), 6-(chloroacetyl)-2H-1,4-benzoxazin-3(4H)-one (2.2 g) and triethylamine (2.2 g) in acetonitrile (40 ml) and DMF (10 ml) was stirred at 60° C. for 14 hr. The mixture was concentrated in vacuo. The residue was dissolved in EtOAc, and the solution was washed with water, dried and concentrated to give crystals (3.48 g). A mixture of the crystals (3.0 g), diisopropylamine (4.6 g) and DMF (50 ml) was stirred at 60° C. for 70 hr. The solvent was removed under reduced pressure. The residue was dissolved in EtOAc, washed with water, dried and concentrated to give crystals. Recrystallization from EtOAc-THF afforded the title compound as colorless crystals (1.5 g).

mp. 233-234° C.

¹H-NMR (300 MHz, DMSO-d₆) δ: 4.61 (2H, s), 5.46 (2H, d, J=52.4 Hz), 6.74 (1H, d, J=2.1 Hz), 6.92-7.03 (2H, m), 7.37 (1H, d, J=8.1 Hz), 7.55 (1H, dd, J=8.1, 2.1 Hz), 7.80 (1H, d, J=2.1 Hz), 10.86 (1H, s).

### Example 12

### 6-(2-Methyl-5-phenyl-1,3-thiazol-4-yl)-2H-1,4-benzoxazin-3(4H)-one

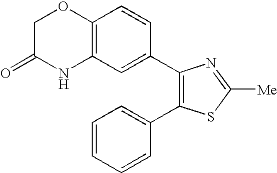

A mixture of 6-[bromo(phenyl)acetyl]-2H-1,4-benzoxazin-3(4H)-one (0.50 g) and thioacetamide (0.11 g) in EtOH was refluxed for 11 hr. The solvent was removed under reduced pressure. The residue was dissolved in EtOAc, and the solution was washed with water, dried and concentrated to give an amorphous solid. Column chromatography on silica gel, followed by washing with IPE gave the title compound as colorless crystals (0.11 g).

mp. 221.0-229.5° C.

¹H-NMR (300 MHz, CDCl₃) δ: 2.74 (3H, s), 4.61 (2H, s), 6.83 (1H, d, J=8.4 Hz), 7.00-7.07 (2H, m), 7.33 (5H, s), 7.63 (1H, brs).

### Example 13

### 6-[2-(Methylamino)-5-phenyl-1,3-thiazol-4-yl]-2H-1,4-benzoxazin-3(4H)-one

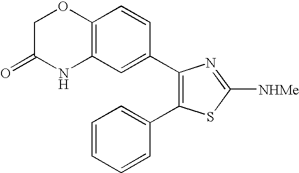

A mixture of 6-[bromo(phenyl)acetyl]-2H-1,4-benzoxazin-3(4H)-one (0.50 g) and N-methylthiourea (0.18 g) in EtOH was refluxed for 2.5 hr. The solvent was removed under reduced pressure. The residue was dissolved in CHCl₃, washed water, dried and concentrated to give an amorphous solid. Column chromatography on silica gel, followed by washing with IPE gave the title compound as colorless crystals (0.12 g).

mp. 236-248° C.

¹H-NMR (300 MHz, DMSO-d₆) δ: 2.90 (3H, d, J=4.8 Hz), 4.51 (2H, s), 6.73 (1H, d, J=8.3 Hz), 6.87 (1H, dd, J=8.3, 2.0 Hz), 7.13 (1H, d, J=2.0 Hz), 7.13-7.31 (5H, m), 7.43 (1H, m), 10.62 (1H, brs).

### Example 14

### 6-(2-Phenyl-2H-1,4-benzothiazin-3-yl)-2H-1,4-benzoxazin-3(4H)-one

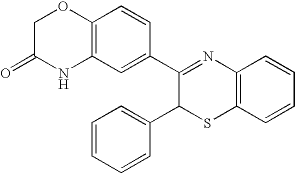

A suspension of 6-[bromo(phenyl)acetyl]-2H-1,4-benzoxazin-3(4H)-one (0.50 g) and 2-amionothiophenol (0.18 g) in EtOH was refluxed for 2.5 hr. The solvent was removed under reduced pressure. The residue was dissolved in CHCl₃, washed water, dried and concentrated to give an amorphous solid. Column chromatography on silica gel, followed by washing with IPE gave the title compound as colorless crystals (0.10 g).

mp. 215-217° C.

¹H-NMR (300 MHz, CDCl₃) δ: 4.67 (2H, s), 5.17 (1H, s), 6.98 (1H, d, J=8.4 Hz), 7.03-7.32 (8H, m), 7.44-7.57 (2H, m), 7.63 (1H, s), 7.98 (1H, brs).

### Example 15

### 6-(6-Chloro-2-phenyl-2H-1,4-benzothiazin-3-yl)-2H-1,4-benzoxazin-3(4H)-one

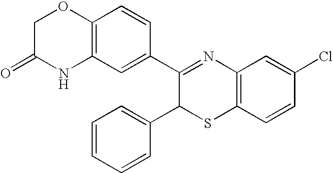

A suspension of 6-[bromo(phenyl)acetyl]-2H-1,4-benzoxazin-3(4H)-one (1.0 g) and 2-amino-4-chlorothiophenol (0.46 g) in EtOH was refluxed for 2.5 hr. The solvent was removed under reduced pressure. The residue was dissolved in CHCl₃, washed water, dried and concentrated to give an amorphous solid. Column chromatography on silica gel, followed by washing with IPE gave the title compound as colorless crystals (0.23 g).

mp. 234-236° C.

¹H-NMR (300 MHz, CDCl₃) δ: 4.68 (2H, s), 5.20 (1H, s), 6.99 (1H, d, J=8.4 Hz), 7.06 (1H, dd, J=8.4, 2.1 Hz), 7.10-7.26 (6H, m), 7.49 (1H, dd, J=8.4, 1.8 Hz), 7.55 (1H, d, J=2.4 Hz), 7.63 (1H, d, J=1.8 Hz), 7.87 (1H, brs).

### Example 16

### 6-(2-Phenylpyridin-3-yl)-2H-1,4-benzoxazin-3(4H)-one

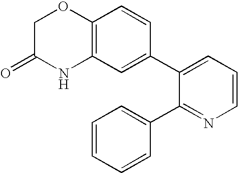

A mixture of 3-bromo-2-phenylpyridine (0.17 g), 6-(4,4,5,5-tetramethyl-1,3,2-dioxaborolan-2-yl)-2H-1,4-benzoxazin-3(4H)-one (0.2 g), tris(dibenzylideneacetone)dipalladium(0) (33.3 mg), 2-dicyclohexylphosphino-2′,4′,6′-triisopropylbiphenyl (34.7 mg) and tripotassium phosphate (0.46 g) in water (1 ml) and DMF (5 ml) was heated at 100° C. for 48 hr. The solvent was removed under reduced pressure. The residue was dissolved in EtOAc, and the solution was washed with aqueous NaHCO₃ and water, dried and concentrated. Column chromatography on silica gel gave crystals. Recrystallization from EtOAc-hexane afforded the title compound as colorless crystals (26 mg).

mp. 176-178° C.

¹H-NMR (300 MHz, CDCl₃) δ: 4.62 (2H, s), 6.55 (1H, d, J=2.4 Hz), 6.81 (1H, dd, J=2.1, 8.4 Hz), 6.90 (1H, d, J=8.1 Hz), 7.23-7.41 (6H, m), 7.68 (1H, dd, J=1.8, 7.8 Hz), 7.80 (1H, br), 8.69 (1H, dd, J=2, 1, 5.1 Hz).

### Example 17

### 6-(2H-1,4-Benzothiazin-3-yl)-2H-1,4-benzoxazin-3(4H)-one

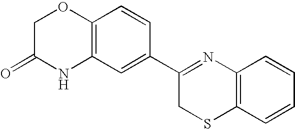

A suspension of 6-[bromoacetyl]-2H-1,4-benzoxazin-3(4H)-one (1.0 g) and 2-amionothiophenol (0.55 g) in EtOH was refluxed for 9 hr. After cooling, the precipitate was collected and washed with EtOH to give the title compound as crystals (1.2 g).

mp. 280-281° C.

¹H-NMR (300 MHz, DMSO-d₆) δ: 3.76 (2H, s), 4.67 (2H, s), 7.06 (1H, d, J=8.4 Hz), 7.10-7.29 (2H, m), 7.34-7.42 (2H, m), 7.65 (1H, d, J=8.1 Hz), 7.74 (1H, d, J=2.1 Hz), 10.86 (1H, s).

### Preparation 13

### 6-(Phenylacetyl)-2H-1,4-benzoxazin-3(4H)-one

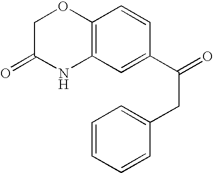

To a suspension of 2H-1,4-benzoxazin-3(4H)-one (70.0 g) in 1,2-dichloroethane (800 mL) was added powdered AlCl₃ (138 g), and the mixture was stirred at room temperature for 5 min to give a solution. The solution was cooled with an water-bath, and then phenylacetyl chloride (75.0 mL) was added dropwise over 0.5 hr. After the addition was completed, the bath was removed. The mixture was stirred at room temperature for 20 hr, poured onto crashed ice and extracted with THF. The extract was washed with brine and saturated aqueous NaHCO₃, dried and concentrated. The residue was suspended in ethyl acetate and collected by filtration. Recrystallization from THF/ethyl acetate gave the title compound (56.6 g).

¹H-NMR (300 MHz, CDCl₃) δ: 4.22 (2H, s), 4.69 (2H, s), 7.00 (1H, d, J=8.4 Hz), 7.22-7.36 (5H, m), 7.48-7.49 (1H, d, J=2.1 Hz), 7.68 (1H, dd, J=8.4, 2.1 Hz), 8.10 (1H, br).

MS m/z: 268 (MH⁺).

### Preparation 14

### 6-[Bromo(phenyl)acetyl]-2H-1,4-benzoxazin-3(4H)-one

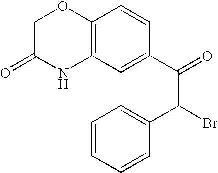

To a suspension of 6-(phenylacetyl)-2H-1,4-benzoxazin-3(4H)-one (25.00 g) in AcOH (280 mL) and 25% hydrogen bromide in acetic acid (70 mL) was added portionwise pyridinium tribromide (30.38 g). The mixture was stirred at room temperature for 0.5 hr and then cooled with an ice bath. Aqueous Na₂S₂O₃ was added dropwise to the mixture, and the whole mixture was diluted with water. The supernatant was decanted, and the residue was treated with ethyl acetate and 10% aqueous citric acid. The organic layer was separated, washed with saturated aqueous NaHCO₃, dried over MgSO₄, passed through silica gel plough and concentrated. The residue was suspended in ethyl acetate/diisopropyl ether and collected by filtration to give the title compound (28.26 g).

¹H-NMR (300 MHz, CDCl₃) δ: 4.70 (2H, s), 6.30 (1H, s), 6.98 (1H, d, J=8.7 Hz), 7.29-7.53 (6H, m), 7.62 (1H, dd, J=8.7, 2.1 Hz), 8.64 (1H, br).

### Example 18

### 2-Phenyl-2H,2′H-3,6′-bi-1,4-benzoxazin-3′(4′H)-one

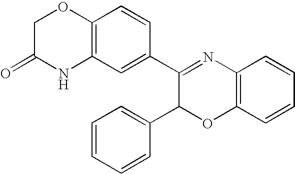

To a mixture of 6-[bromo(phenyl)acetyl]-2H-1,4-benzoxazin-3(4H)-one (0.50 g) and 2-aminophenol (0.16 g) in acetone (10 mL) and THF (2 mL) was added potassium carbonate (0.42 g). The mixture was refluxed for 2 hr and concentrated, and the residue was treated with ethyl acetate and water. The organic layer was separated, dried over MgSO₄ and concentrated. The residue was chromatographed on silica gel using ethyl acetate/n-hexane as an eluent and followed by recrystallization from ethanol to give the title compound as colorless crystals (0.01 g).

¹H-NMR (300 MHz, CDCl₃) δ: 4.66 (2H, s), 6.27 (1H, s), 6.81-7.11 (4H, m), 7.26-7.45 (7H, m), 7.62 (1H, d, J=2.4 Hz), 7.70 (1H, brs).

### Example 19

### 6-(2-Phenyl-2H-chromen-3-yl)-2H-1,4-benzoxazin-3(4H)-one

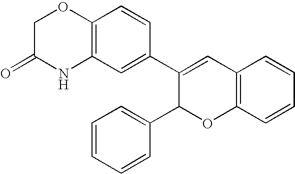

To a suspension of 2-hydroxybenzyltriphenylphosphonium bromide (0.65 g, Tetrahedron Lett., 1979, 23, 2145) in toluene (6 mL) was added 2.5 M sodium methoxide solution in methanol (0.58 mL) and the mixture was stirred at room temperature for 10 min. Then 6-[bromo(phenyl)acetyl]-2H-1,4-benzoxazin-3(4H)-one (0.50 g) was added and the mixture was refluxed for 0.5 hr. An 2.5 M sodium methoxide solution in methanol (0.58 mL) was added and the whole mixture was refluxed for an additional 6 hr, cooled and treated with ethyl acetate and water. The organic layer was separated, dried over MgSO₄ and concentrated. The residue was chromatographed on silica gel using ethyl acetate/n-hexane as an eluent and followed by recrystallization from ethyl acetate/n-hexane to give the title compound as colorless crystals (0.09 g).

mp. 246-249° C.

¹H-NMR (300 MHz, DMSO-d₆) δ: 4.60 (2H, s), 6.38 (1H, s), 6.71 (1H, d, J=8.4 Hz), 6.85-6.93 (2H, m), 7.04-7.38 (10H, m), 10.68 (1H, brs).

### Example 20

### 6-(2-Phenyl-2H-thiochromen-3-yl)-2H-1,4-benzoxazin-3(4H)-one

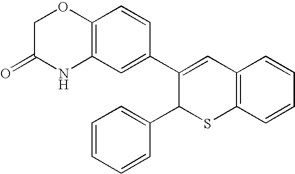

To a suspension of 2-mercaptobenzyltriphenylphosphonium bromide (0.67 g, Synthesis, 1988, 2, 155) in toluene (6 mL) was added 2.5 M sodium methoxide solution in methanol (0.58 mL) and the mixture was stirred at room temperature for 10 min. Then 6-[bromo(phenyl)acetyl]-2H-1,4-benzoxazin-3(4H)-one (0.50 g) was added and the mixture was refluxed for 0.5 hr. An 2.5 M sodium methoxide solution in methanol (0.58 mL) was added and the whole mixture was refluxed for an additional 6 hr, cooled and treated with ethyl acetate and water. The organic layer was separated, dried over MgSO₄ and concentrated. The residue was chromatographed on silica gel using ethyl acetate/n-hexane as an eluent and followed by recrystallization from ethyl acetate to give the title compound as colorless crystals (0.19 g).

mp. 226-227° C.

¹H-NMR (300 MHz, DMSO-d₆) δ: 4.56 (2H, s), 5.30 (1H, s), 6.92 (1H, d, J=8.7 Hz), 7.06-7.27 (11H, m), 7.43 (1H, d, J=6.6 Hz), 10.70 (1H, brs).

### Examples 21 and 22

### 6-(1,1-Dioxido-2-phenyl-2H-thiochromen-3-yl)-2H-1,4-benzoxazin-3(4H)-one (Example 21) and 6-(1-oxido-2-phenyl-2H-thiochromen-3-yl)-2H-1,4-benzoxazin-3(4H)-one (Example 22)

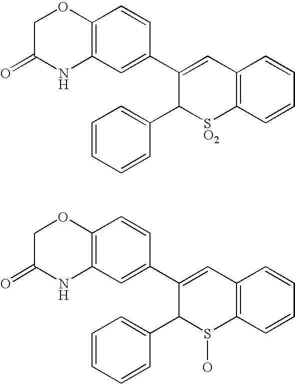

To a solution of 6-(2-phenyl-2H-thiochromen-3-yl)-2H-1,4-benzoxazin-3(4H)-one (86 mg) in acetonitrile/DMF (2/1, 3 mL) was added 65% 3-chloroperbenzoic acid (61 mg) with ice-cooling. The mixture was stirred at 0° C. for 3 hr and treated with ethyl acetate and 10% aqueous Na₂S₂O₃. The organic layer was separated, washed with 10% aqueous citric acid and saturated aqueous NaHCO₃, dried over MgSO₄ and concentrated. The residue was chromatographed on silica gel using hexane/ethyl acetate as an eluent to give 6-(1,1-dioxido-2-phenyl-2H-thiochromen-3-yl)-2H-1,4-benzoxazin-3(4H)-one as colorless crystals (9 mg) and 6-(1-oxido-2-phenyl-2H-thiochromen-3-yl)-2H-1,4-benzoxazin-3(4H)-one as colorless crystals (46 mg).

### 6-(1,1-Dioxido-2-phenyl-2H-thiochromen-3-yl)-2H-1,4-benzoxazin-3(4H)-one

¹H-NMR (300 MHz, CDCl₃) δ: 4.60 (2H, s), 5.16 (1H, s), 6.88-6.90 (2H, m), 7.00 (1H, dd, J=8.4, 2.1 Hz), 7.18 (1H, s), 7.25-7.30 (5H, m), 7.39-7.48 (2H, m), 7.62 (1H, dt, J=7.5, 1.2 Hz), 7.80 (1H, d, J=7.5 Hz), 8.67 (1H, brs).

### 6-(1-Oxido-2-phenyl-2H-thiochromen-3-yl)-2H-1,4-benzoxazin-3(4H)-one

mp. 228° C.

¹H-NMR (300 MHz, CDCl₃) δ: 4.51 (1H, d, J=15.3 Hz), 4.56 (1H, d, J=15.3 Hz), 5.52 (1H, s), 6.85 (1H, d, J=8.4 Hz), 6.93 (1H, d, J=2.1 Hz), 6.99 (1H, dd, J=8.4, 2.1 Hz), 7.01-7.35 (7H, m), 7.47-7.58 (3H, m), 8.32 (1H, brs).

### Preparation 15

### (2-hydroxy-4-methoxybenzyl)(triphenyl)phosphonium bromide

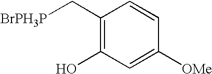

A mixture of 2-(hydroxymethyl)-5-methoxyphenol (1.00 g) and triphenylphosphine hydrobromide (2.23 g) in acetonitrile (25 mL) was refluxed for 14 hr and concentrated. The residue was crystallized from acetonitrile/ethyl acetate and the crystals were collected to give the title compound (1.95 g).

¹H-NMR (300 MHz, CDCl₃) δ: 3.64 (3H, s), 4.49 (2H, d, J=12.6 Hz), 6.19 (1H, dd, J=8.4, 2.7 Hz), 6.72 (1H, dd, J=8.4, 2.7 Hz), 6.97 (1H, d, J=2.7 Hz), 7.26-7.76 (15H, m), 9.16 (1H, s).

### Example 23

### 6-(7-Methoxy-2-phenyl-2H-chromen-3-yl)-2H-1,4-benzoxazin-3(4H)-one

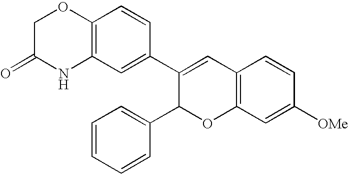

The title compound was obtained from 6-[bromo(phenyl)acetyl]-2H-1,4-benzoxazin-3(4H)-one (0.69 g) and (2-hydroxy-4-methoxybenzyl)(triphenyl)phosphonium bromide (1.15 g) according to a method similar to the procedure for 6-(2-phenyl-2H-chromen-3-yl)-2H-1,4-benzoxazin-3(4H)-one as colorless crystals (0.03 g).

mp. 229° C.

¹H-NMR (300 MHz, CDCl₃) δ: 3.73 (3H, s), 4.60 (2H, s), 6.16 (1H, s), 6.34 (1H, d, J=2.4 Hz), 6.45 (1H, dd, J=8.4, 2.4 Hz), 6.77 (1H, d, J=1.8 Hz), 6.90 (1H, d, J=8.4 Hz), 6.99-7.06 (3H, m), 7.26-7.31 (3H, m), 7.41-7.44 (2H, m), 7.75 (1H, brs).

### Preparation 16

### Ethyl (4-bromo-2-nitrophenoxy)acetate

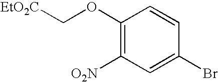

To a mixture of 4-bromo-2-nitrophenol (24.8 g) and potassium carbonate (31.5 g) in DMSO (200 mL) was added ethyl bromoacetate (12.8 mL) dropwise with ice-cooling. The mixture was stirred at room temperature for 16 hr and then treated with ethyl acetate and water. The organic layer was separated, washed with 5% aqueous Na₂S₂O₃, water and saturated aqueous NaHCO₃, dried over MgSO₄ and concentrated to give the title compound as an oil (28.0 g).

¹H-NMR (300 MHz, CDCl₃) δ: 1.29 (3H, t, J=7.2 Hz), 4.27 (2H, q, J=7.2 Hz), 4.76 (2H, s), 6.90 (1H, d, J=9.0 Hz), 7.62 (1H, dd, J=9.0, 2.4 Hz), 8.01 (1H, d, J=2.4 Hz).

### Preparation 17

### 6-Bromo-2H-1,4-benzoxazin-3(4H)-one

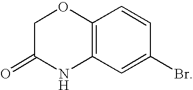

To a mixture of ethyl (4-bromo-2-nitrophenoxy)acetate (28 g), acetic acid (200 mL) and toluene (100 mL) was added portionwise zinc powder (100 g) in a water bath (exothermal reaction initiated). After all of zinc was added, the mixture was stirred for 5 min. Then the bath was removed, and the mixture was heated at 80° C. for 1 hr. The insoluble material was filtered off through celite and the filtered cake was washed with THF. The filtrate was concentrated to dryness, and the resulting crystals were suspended in ethyl acetate and collected by filtration and washed with diisopropyl ether to give the title compound (18.4 g).

¹H-NMR (300 MHz, CDCl₃) δ: 4.62 (2H, s), 6.86 (1H, d, J=8.4 Hz), 6.97 (1H, d, J=2.1 Hz), 7.09 (1H, dd, J=8.4, 2.1 Hz), 6.66 (1H, br).

### Preparation 18

### 6-(4,4,5,5-Tetramethyl-1,3,2-dioxaborolan-2-yl)-2H-1,4-benzoxazin-3(4H)-one

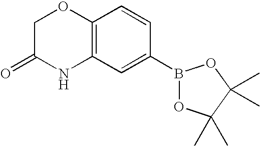

A mixture of 6-bromo-2H-1,4-benzoxazin-3(4H)-one (5.00 g), bis(pinacolato)diboron (5.84 g), [1,1-bis(diphenylphosphino)ferrocene]dichloropalladium(II) dichloromethane adduct (0.54 g) and potassium acetate (8.34 g) in DMF (100 mL) was heated at 60° C. for 16 hr under a nitrogen atmosphere. Then [1,1-bis(diphenylphosphino)ferrocene]dichloropalladium(II) dichloromethane adduct (1.08 g) was added, and the mixture was stirred at 60° C. for an additional 62 hr and treated with ethyl acetate and water. The insoluble material was filtered off, and the organic layer was separated, washed with water, dried over MgSO₄ and concentrated. The residue was chromatographed on silica gel using hexane/ethyl acetate as an eluent to give the title compound as colorless crystals (0.58 g).

¹H-NMR (300 MHz, CDCl₃) δ: 1.33 (12H, s), 4.64 (2H, s), 6.96 (1H, d, J=7.8 Hz), 7.21 (1H, d, J=1.2 Hz), 7.44 (1H, dd, J=7.8, 1.2 Hz), 7.90 (1H, br).

### Preparation 19

### (2E)-2-(3-Oxo-3,4-dihydro-2H-1,4-benzoxazin-6-yl)-3-phenylacrylaldehyde

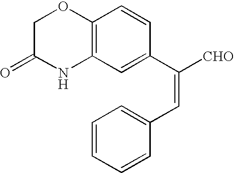

A mixture of α-bromocinnamaldehyde (0.53 g), 6-(4,4,5,5-tetramethyl-1,3,2-dioxaborolan-2-yl)-2H-1,4-benzoxazin-3(4H)-one (0.58 g), [1,1-bis(diphenylphosphino)ferrocene]dichloropalladium(II) dichloromethane adduct (0.33 g), 2M Cs₂CO₃ (4.0 mL) and THF (20 mL) was refluxed for 14 hr, and then treated with ethyl acetate and water. The organic layer was separated, dried over MgSO₄ and concentrated. The residue was chromatographed on silica gel using hexane/ethyl acetate as an eluent to give the title compound as colorless crystals (0.34 g).

mp. 200° C. (decomp.).

¹H-NMR (300 MHz, CDCl₃) δ: 4.65 (2H, s), 6.77 (1H, d, J=1.5 Hz), 6.80 (1H, dd, J=8.4, 1.5 Hz), 6.99 (1H, d, J=8.4 Hz), 7.23-7.33 (5H, m), 7.38 (1H, s), 8.00 (1H, br), 9.73 (1H, s).

### Example 24

### 6-(2-Amino-6-phenyl-6H-1,3-thiazin-5-yl)-2H-1,4-benzoxazin-3(4H)-one

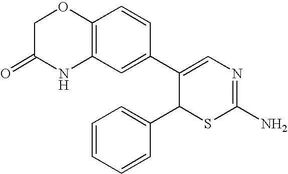

A mixture of (2E)-2-(3-oxo-3,4-dihydro-2H-1,4-benzoxazin-6-yl)-3-phenylacrylaldehyde (116 mg), thiourea (42 mg), 1,4-dioxane (6 mL), water (0.6 mL) and c-HCl (0.6 mL) was heated at 100° C. for 4 hr, and then treated with THF and saturated aqueous NaHCO₃. The organic layer was separated, dried over MgSO₄ and concentrated to give the title compound as colorless crystals (132 mg).

¹H-NMR (300 MHz, DMSO-d₆) δ: 4.50 (2H, s), 5.19 (1H, s), 6.81-6.89 (5H, m), 7.18-7.29 (6H, m), 10.62 (1H, s).

### Example 25

### 6-(2-Methyl-7-phenyl-7H-imidazo[2,1-b][1,3]thiazin-6-yl)-2H-1,4-benzoxazin-3(4H)-one

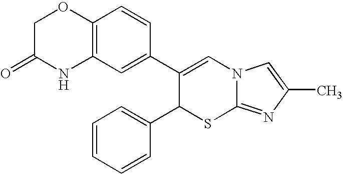

A mixture of 6-(2-amino-6-phenyl-6H-1,3-thiazin-5-yl)-2H-1,4-benzoxazin-3(4H)-one (33 mg) and bromoacetone (0.034 mL) in 1,4-dioxane/ethanol (3/1, 4 mL) was heated at 100° C. for 14 hr, and treated with ethyl acetate and saturated aqueous NaHCO₃. The organic layer was separated, dried over MgSO₄ and concentrated, and the residue was chromatographed on silica gel using hexane/ethyl acetate as an eluent to give the title compound as a foam (7 mg).

¹H-NMR (300 MHz, CDCl₃) δ: 2.18 (3H, s), 4.59 (2H, s), 4.92 (1H, s), 6.75 (1H, s), 6.87-6.90 (3H, m), 7.19-7.27 (6H, m), 8.91 (1H, br).

### Example 26

### 6-(7-Phenyl-7H-imidazo[2,1-b][1,3]thiazin-6-yl)-2H-1,4-benzoxazin-3(4H)-one

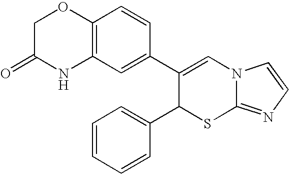

A mixture of 6-(2-amino-6-phenyl-6H-1,3-thiazin-5-yl)-2H-1,4-benzoxazin-3(4H)-one (107 mg) and 45% chloroacetaldehyde solution (0.42 g) in dimethoxyethane/ethanol (6/1, 7 mL) was heated at 100° C. for 12 hr, and treated with ethyl acetate and saturated aqueous NaHCO₃. The organic layer was separated, dried over MgSO₄ and concentrated, and the residue was chromatographed on silica gel using hexane/ethyl acetate as an eluent to give the title compound. Recrystallization from THF/ethyl acetate gave colorless crystals (32 mg).

¹H-NMR (300 MHz, DMSO-d₆) δ: 4.56 (2H, s), 5.53 (1H, s), 6.92-6.97 (3H, m), 7.05 (1H, dd, J=8.4, 2.1 Hz), 7.21-7.32 (5H, m), 7.58 (1H, s), 7.81 (1H, s), 10.77 (1H, s).

### Preparation 20

### 2-(Hydroxymethyl)-5-iodophenol

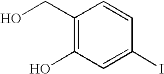

To a solution of 2-hydroxy-4-iodobenzoic acid (3.96 g) in THF (100 mL) was added 1M borane-THF complex in THF (56 mL). The mixture was stirred at room temperature for 2 hr and at 50° C. for 1 hr, cooled and then quenched by the addition of 1N HCl. The mixture was extracted with ethyl acetate, and the extract was dried over MgSO₄, passed through silica gel plough and concentrated. The residue was collected and washed with diisopropyl ether to give the title compound as colorless crystals (2.30 g).

¹H-NMR (300 MHz, CDCl₃) δ: 2.19 (1H, br), 4.84 (2H, d, J=3.0 Hz), 6.75 (1H, d, J=7.8 Hz), 7.19 (1H, dd, J=7.8, 1.8 Hz), 7.44 (1H, d, J=1.8 Hz).

### Preparation 21

### (2-hydroxy-4-iodobenzyl)(triphenyl)phosphonium bromide

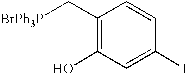

A mixture of 2-(hydroxymethyl)-5-iodophenol (2.28 g) and triphenylphosphine hydrobromide (3.11 g) in acetonitrile (35 mL) was refluxed for 2 hr and concentrated. The crystals were collected and washed with ethyl acetate to give the title compound (4.88 g).

¹H-NMR (300 MHz, DMSO-d₆) δ: 4.87 (2H, d, J=15.0 Hz), 6.62 (1H, dd, J=7.8, 2.7 Hz), 7.00 (1H, d, J=7.8 Hz), 7.05 (1H, d, J=2.7 Hz), 7.67-7.91 (15H, m), 10.15 (1H, s).

### Example 27

### 6-(7-Iodo-2-phenyl-2H-chromen-3-yl)-2H-1,4-benzoxazin-3(4H)-one

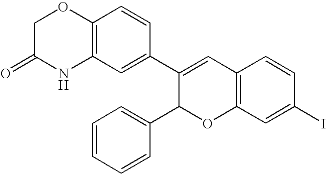

The title compound was obtained from 6-[bromo(phenyl)acetyl]-2H-1,4-benzoxazin-3(4H)-one (2.08 g) and (2-hydroxy-4-iodobenzyl)(triphenyl)phosphonium bromide (3.74 g) according to a method similar to the procedure for 6-(2-phenyl-2H-chromen-3-yl)-2H-1,4-benzoxazin-3(4H)-one as colorless crystals (1.02 g).

¹H-NMR (300 MHz, DMSO-d₆) δ: 4.56 (2H, s), 6.41 (1H, s), 6.92 (1H, d, J=4.2 Hz), 7.03-7.08 (11H, m), 10.71 (1H, s).

MS m/z: 482 (MH⁺).

### Example 28

### 3-(3-Oxo-3,4-dihydro-2H-1,4-benzoxazin-6-yl)-2-phenyl-2H-chromene-7-carbonitrile

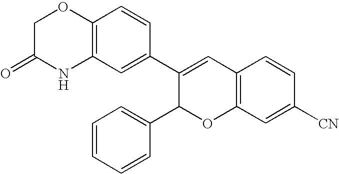

A mixture of 6-(7-iodo-2-phenyl-2H-chromen-3-yl)-2H-1,4-benzoxazin-3(4H)-one (0.34 g), Zn(CN)₂ (0.12 g) and Pd(PPh₃)₄ (0.08 g) in DMF (5 mL) was heated at 85° C. for 16 hr under a nitrogen atmosphere. The mixture was treated with water and ethyl acetate, and the organic layer was separated, washed with water, dried and concentrated. The residue was chromatographed on silica gel using hexane/ethyl acetate as an eluent to give the title compound. Crystallization from ethyl acetate gave colorless crystals (0.19 g).

¹H-NMR (300 MHz, CDCl₃) δ: 4.60 (2H, s), 6.26 (1H, s), 6.87-7.37 (12H, m), 9.60-9.80 (1H, br). MS m/z: 381 (MH⁺).

### Preparation 22

### 1-(4-Methoxy-3-nitrophenyl)-2-phenylethanone

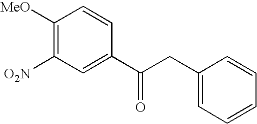

To a suspension of 4-methoxy-3-nitrobenzoic acid (9.00 g) in CH₂Cl₂/DMF (150 ml/4.0 ml) was added oxalyl chloride (4.2 ml) at room temperature. After stirring for 2 hr at room temperature, the reaction solvent was removed in vacuo. The residue was suspended in THF (150 ml). To this suspension were added tetrakis(triphenylphosphine)palladium (0.90 g) and benzylzinc bromide (100 ml, 0.5 M THF solution) under N₂ atmosphere. After stirring for 12 hr at room temperature, the reaction mixture was diluted with ethyl acetate and water. The resulting mixture was extracted with ethyl acetate. The organic extract was washed with 1N NaOH and brine, dried over Na₂SO₄ and concentrated in vacuo. The residue was chromatographed on silica gel using hexane/ethyl acetate as an eluent to give the title compound (3.70 g).

¹H-NMR (300 MHz, DMSO-d₆) δ: 4.02 (3H, s), 4.41 (2H, s), 7.20-7.36 (5H, m), 7.50 (1H, d, J=9.0 Hz), 8.32 (1H, dd, J=9.0, 2.5 Hz), 8.50 (1H, d, J=2.5 Hz).

### Preparation 23

### 1-(4-Hydroxy-3-nitrophenyl)-2-phenylethanone

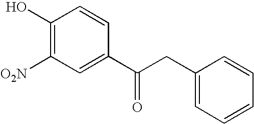

To a solution of 1-(4-methoxy-3-nitrophenyl)-2-phenylethanone (2.20 g) in CH₂Cl₂ (30 ml) was added BBr₃ (24.4 ml, 1.0 M CH₂Cl₂ solution) at −78° C. After stirring for 5 hr at −20 to −15° C., the reaction mixture was quenched with MeOH at −78° C. The mixture was diluted with ethyl acetate and water. The resulting mixture was extracted with ethyl acetate. The organic extract was washed with sat. NH₄Cl and brine, dried over Na₂SO₄ and concentrated in vacuo. The residue was chromatographed on silica gel using hexane/ethyl acetate as an eluent to give the title compound (1.77 g).

¹H-NMR (300 MHz, DMSO-d₆) δ: 4.36 (2H, s), 7.13-7.38 (6H, m), 8.16 (1H, dd, J=8.5, 2.0 Hz), 8.51 (1H, d, J=2.0 Hz), 12.04 (1H, s).

### Preparation 24

### 5-(Phenylacetyl)-1,3-benzoxazol-2(3H)-one

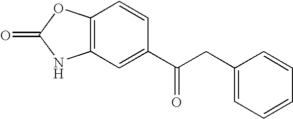

A suspension of 1-(4-hydroxy-3-nitrophenyl)-2-phenylethanone (1.77 g) and 10% Pd—C (100 mg) in MeOH (25 ml) was stirred for 2 hr at room temperature under H₂ (3 kgf/cm²). The reaction mixture was filtered through filter paper and the filtrate was concentrated in vacuo. The residue was dissolved in THF (100 ml). To this solution was added N,N′-carbonyldiimidazole (5.60 g) at room temperature. After stirring for 13 hr at room temperature, the reaction solvent was removed in vacuo. The residue was dissolved in a mixture of ethyl acetate and water. The resulting mixture was extracted with ethyl acetate. The organic extract was washed with sat. NH₄Cl and brine, dried over Na₂SO₄ and concentrated in vacuo. The residue was chromatographed on silica gel using hexane/ethyl acetate as an eluent to give the title compound (790 mg).

¹H-NMR (300 MHz, DMSO-d₆) δ: 4.39 (2H, s), 7.15-7.36 (5H, m), 7.41 (1H, d, J=8.5 Hz), 7.64 (1H, s), 7.88 (1H, d, J=8.5 Hz), 11.89 (1H, s).

### Example 29

### 5-(7-Phenyl-7H-[1,2,4]triazolo[3,4-b][1,3,4]thiadiazin-6-yl)-1,3-benzoxazol-2(3H)-one

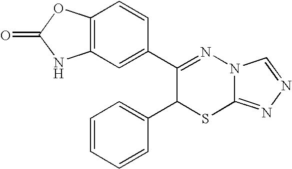

To a solution of 5-(phenylacetyl)-1,3-benzoxazol-2(3H)-one (790 mg) in AcOH (60 ml) were added 25% HBr—AcOH solution (15 ml) and pyridine hydrobromide perbromide (1.10 g) at room temperature. After stirring for 3.5 hr at room temperature, the reaction mixture was diluted with ethyl acetate and water. The resulting mixture was extracted with ethyl acetate. The organic extract was washed with brine, dried over Na₂SO₄ and concentrated in vacuo. The residue was suspended in EtOH/toluene (60 ml/30 ml). To this suspension was added 4-amino-4H-1,2,4-triazole-3-thiol (400 mg) at room temperature. After stirring for 12 hr under reflux, the reaction mixture was diluted with ethyl acetate, THF and sat. NaHCO₃. The resulting mixture was extracted with a mixture of ethyl acetate and THF. The organic extract was washed with brine, dried over Na₂SO₄ and concentrated in vacuo. The residue was purified by HPLC using water/acetonitrile as an eluent to give the title compound (267 mg).

¹H-NMR (300 MHz, DMSO-d₆) δ: 6.45 (1H, s), 7.13-7.20 (2H, m), 7.24-7.45 (4H, m), 7.55-7.71 (2H, m), 9.28 (1H, s), 11.87 (1H, s)

### Example 30

### 6-(7H-[1,2,4]Triazolo[3,4-b][1,3,4]thiadiazin-6-yl)-2H-1,4-benzoxazin-3(4H)-one

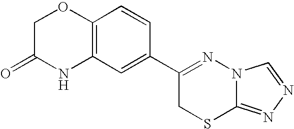

A mixture of 6-(chloroacetyl)-2H-1,4-benzoxazin-3(4H)-one (2.0 g) and 4-amino-3-mercapto-4H-1,2,4-triazole (1.1 g), ethanol (40 ml) and toluene (20 ml) was refluxed for 24 hr and then 4-amino-3-mercapto-4H-1,2,4-triazole (0.2 g) was added to the mixture. The mixture was refluxed for 12 hr. Methanol (300 ml) and 3% aqueous potassium carbonate (100 ml) were added to the mixture and then methanol was removed in vacuo. The resulting crystals were collected by filtration and suspended in ethanol and the mixture was refluxed for 6 hr. After cooling the mixture, the resulting crystals were collected by filtration. The crystals were suspended in methanol and the mixture was refluxed for 1 hr. After cooling the mixture, the resulting crystals were collected by filtration. The title compound was obtained as crystals (2.07 g).

mp. 273-274° C. (decomp.).

¹H-NMR (300 MHz, DMSO-d₆) δ: 4.38 (2H, s), 4.70 (2H, s), 7.12 (1H, d, J=9.0 Hz), 7.51-7.59 (2H, m), 9.14 (1H, s), 10.95 (1H, s).

### Example 31

### 6-(7-Propyl-7H-[1,2,4]triazolo[3,4-b][1,3,4]thiadiazin-6-yl)-2H-1,4-benzoxazin-3(4H)-one

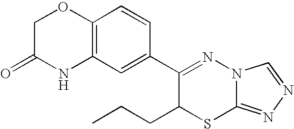

A mixture of 6-(2-bromopentanoyl)-2H-1,4-benzoxazin-3(4H)-one (0.5 g), 4-amino-4H-1,2,4-triazole-3-thiol (0.186 g), ethanol (10 ml) and toluene (5 ml) was refluxed for 12 hr and then concentrated in vacuo. Water and saturated NaHCO₃ aqueous solution were added and the mixture was extracted with ethyl acetate. The organic layer was washed with water and brine, dried over Na₂SO₄ and concentrated in vacuo. The residue was crystallized from methanol to give the title compound as crystals (0.34 g).

mp. 235-237° C.

¹H-NMR (300 MHz, DMSO-d₆) δ: 0.85 (3H, t, J=7.0 Hz), 1.20-1.66 (4H, m), 4.70 (2H, s), 4.87 (1H, dd, J=9.1, 5.0 Hz), 7.13 (1H, d, J=9.2 Hz), 7.54-7.62 (2H, m), 9.17 (1H, s), 10.96 (1H, s).

### Example 32

### 6-[7-(4-Chlorophenyl)-7H-[1,2,4]triazolo[3,4-b][1,3,4]thiadiazin-6-yl]-2H-1,4-benzoxazin-3(4H)-one

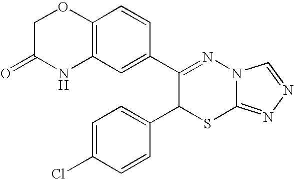

A mixture of 6-[bromo(4-chlorophenyl)acetyl]-2H-1,4-benzoxazin-3(4H)-one (0.5 g), 4-amino-4H-1,2,4-triazole-3-thiol (0.152 g), ethanol (10 ml) and toluene (5 ml) was refluxed for 12 hr and then concentrated in vacuo. Water and saturated NaHCO₃ aqueous solution were added and the mixture was extracted with a solution of THF and ethyl acetate. The organic layer was washed with water and brine, dried over Na₂SO₄ and concentrated in vacuo. The residue was purified by chromatography on basic silica gel (ethyl acetate) followed by crystallization from THF/ethyl acetate to give the title compound as crystals (380 mg).

mp. 174-176° C.

¹H-NMR (300 MHz, DMSO-d₆) δ: 4.68 (2H, s), 6.35 (1H, s), 7.09 (1H, d, J=8.48 Hz), 7.18 (2H, d, J=8.58 Hz), 7.41 (2H, d, J=8.58 Hz), 7.46 (1H, dd, J=8.48, 2.26 Hz), 7.57 (1H, d, J=2.26 Hz), 9.26 (1H, s), 10.95 (1H, s).

### Example 33

### 6-[7-(3-Chlorophenyl)-7H-[1,2,4]triazolo[3,4-b][1,3,4]thiadiazin-6-yl]-2H-1,4-benzoxazin-3(4H)-one

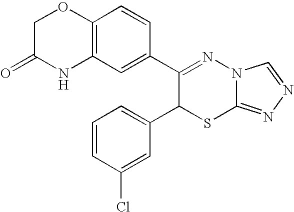

The title compound was obtained as crystals (0.75 g) from 6-[bromo(3-chlorophenyl)acetyl]-2H-1,4-benzoxazin-3(4H)-one (0.8 g) according to a method similar to the procedure for 6-[7-(4-chlorophenyl)-7H-[1,2,4]triazolo[3,4-b][1,3,4]thiadiazin-6-yl]-2H-1,4-benzoxazin-3(4H)-one.

mp. 144-146° C. (THF/ethyl acetate).

¹H-NMR (300 MHz, DMSO-d₆) δ: 4.68 (2H, s), 6.35 (1H, s), 7.00 (1H, d, J=7.5 Hz), 7.10 (1H, d, J=8.6 Hz), 7.30-7.43 (3H, m), 7.47 (1H, dd, J=8.6, 2.2 Hz), 7.57 (1H, d, J=2.2 Hz), 9.29 (1H, s), 10.95 (1H, s).

### Example 34

### 6-[7-(2-Chlorophenyl)-7H-[1,2,4]triazolo[3,4-b][1,3,4]thiadiazin-6-yl]-2H-1,4-benzoxazin-3(4H)-one

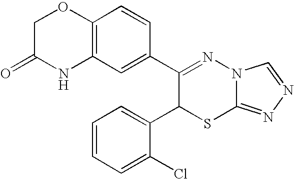

A mixture of 6-[bromo(2-chlorophenyl)acetyl]-2H-1,4-benzoxazin-3(4H)-one (0.8 g), 4-amino-4H-1,2,4-triazole-3-thiol (0.244 g), ethanol (16 ml) and toluene (8 ml) was refluxed for 12 hr and then concentrated in vacuo. Water and saturated aqueous sodium bicarbonate solution were added to the mixture, and the mixture was extracted with a solution of THF and ethyl acetate. The organic layer was washed with water and brine, dried over Na₂SO₄ and concentrated in vacuo. The residue was crystallized from THF/ethyl acetate to give the title compound as crystals (0.56 g).

mp. 234-235° C. (THF/ethyl acetate).

¹H-NMR (300 MHz, DMSO-d₆) δ: 4.66 (2H, s), 6.28 (1H, s), 6.73 (1H, dd, J=7.7, 1.5. Hz), 7.07 (1H, d, J=8.5 Hz), 7.16-7.25 (1H, m), 7.33-7.43 (2H, m), 7.50 (1H, d, J=2.1 Hz), 7.63-7.69 (1H, m), 9.30 (1H, s), 10.95 (1H, s).

### Example 35

### 6-[7-(4-Fluorophenyl)-7H-[1,2,4]triazolo[3,4-b][1,3,4]thiadiazin-6-yl]-2H-1,4-benzoxazin-3(4H)-one

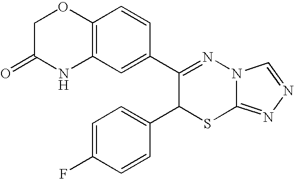

The title compound was obtained as crystals (0.36 g) from 6-[bromo(4-fluorophenyl)acetyl]-2H-1,4-benzoxazin-3(4H)-one (0.5 g) according to a method similar to the procedure for 6-[7-(4-chlorophenyl)-7H-[1,2,4]triazolo[3,4-b][1,3,4]thiadiazin-6-yl]-2H-1,4-benzoxazin-3(4H)-one.

mp. 153-155° C. (ethyl acetate).

¹H-NMR (300 MHz, DMSO-d₆) δ: 4.68 (2H s) 6.35 (1H s) 7.09 (1H d, J=8.5 Hz) 7.12-7.26 (4H m) 7.46 (1H dd, J=8.5, 2.3 Hz) 7.58 (1H d, J=2.3 Hz) 9.27 (1H s) 10.95 (1H s)

### Example 36

### 6-(7-Benzyl-7H-[1,2,4]triazolo[3,4-b][1,3,4]thiadiazin-6-yl)-2H-1,4-benzoxazin-3(4H)-one

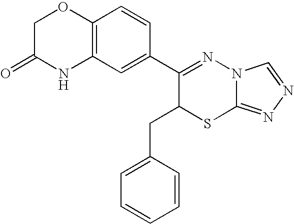

The title compound was obtained as crystals (0.41 g) from 6-(2-bromo-3-phenylpropanoyl)-2H-1,4-benzoxazin-3(4H)-one (0.40 g) according to a method similar to the procedure for 6-[7-(4-chlorophenyl)-7H-[1,2,4]triazolo[3,4-b][1,3,4]thiadiazin-6-yl]-2H-1,4-benzoxazin-3(4H)-one.

mp. 199-201° C. (ethyl acetate).

¹H-NMR (300 MHz, DMSO-d₆) δ: 2.77 (1H, dd, J=14.1, 9.2 Hz), 3.01 (1H, dd, J=14.1, 5.7 Hz), 4.69 (2H, s), 5.14 (1H, dd, J=9.2, 5.7 Hz), 7.06 (1H, d, J=8.6 Hz), 7.11-7.31 (5H, m), 7.51 (1H, dd, J=8.6, 2.2 Hz), 7.58 (1H, d, J=2.2 Hz), 9.12 (1H, s), 10.95 (1H, s).

### Example 37

### 6-(7-Methyl-7-phenyl-7H-[1,2,4]triazolo[3,4-b][1,3,4]thiadiazin-6-yl)-2H-1,4-benzoxazin-3(4H)-one

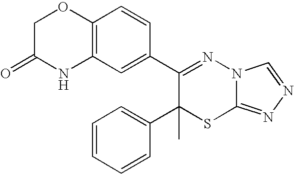

A mixture of 6-(2-bromo-2-phenylpropanoyl)-2H-1,4-benzoxazin-3(4H)-one (0.3 g), 4-amino-4H-1,2,4-triazole-3-thiol (0.29 g), triethylamine (3 ml) and ethanol (3 ml) was stirred at 80° C. for 6 hr and then concentrated in vacuo. Water and saturated NaHCO₃ aqueous solution were added and the mixture was extracted with ethyl acetate. The organic layer was washed with water and brine, dried over Na₂SO₄ and concentrated in vacuo. The residue was purified by chromatography on silica gel (ethyl acetate→ethyl acetate:methanol=20:1) to give the title compound as an amorphous solid (0.2 g).

¹H-NMR (300 MHz, DMSO-d₆) δ: 2.02 (3H, s), 4.62 (2H, s), 6.86-6.94 (2H, m), 7.05 (1H, s), 7.28-7.49 (5H, m), 9.26 (1H, s), 10.75 (1H, s).

### Example 38

### 6-(7-Pyridin-2-yl-7H-[1,2,4]triazolo[3,4-b][1,3,4]thiadiazin-6-yl)-2H-1,4-benzoxazin-3(4H)-one

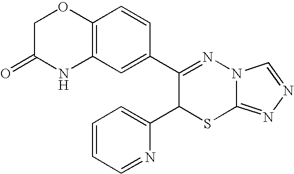

A mixture of 6-[bromo(pyridin-2-yl)acetyl]-2H-1,4-benzoxazin-3(4H)-one hydrobromide (0.3 g), 4-amino-4H-1,2,4-triazole-3-thiol (0.1 g), triethylamine (1 ml) and ethanol (6 ml) was stirred at 80° C. for 3 hr and then THF (6 ml) was added. The mixture was stirred at 80° C. for 4 hr and then concentrated in vacuo. Water and saturated NaHCO₃ aqueous solution were added and the mixture was extracted with ethyl acetate. The organic layer was washed with water and brine, dried over Na₂SO₄ and concentrated in vacuo. The residue was purified by chromatography on basic silica gel (ethyl acetate→THF) followed by crystallization from THF/ethyl acetate to give the title compound as crystals (28 mg).

mp. 216-218° C.

¹H-NMR (300 MHz, DMSO-d₆) δ: 4.66 (2H, s), 6.36 (1H, s), 7.06 (1H, d, J=8.6 Hz), 7.26-7.34 (1H, m), 7.48 (1H, dd, J=8.6, 2.0 Hz), 7.57 (1H, d, J=2.0 Hz), 7.63 (1H, d, J=7.6 Hz), 7.81-7.90 (1H, m), 8.25-8.31 (1H, m), 9.21 (1H, s), 10.92 (1H, s).

### Example 39

### 6-[2-(4-Chlorophenyl)-2H-1,4-benzothiazin-3-yl]-2H-1,4-benzoxazin-3(4H)-one

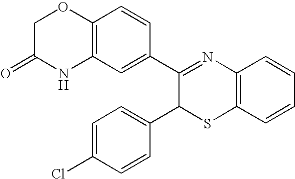

A mixture of 6-[bromo(4-chlorophenyl)acetyl]-2H-1,4-benzoxazin-3(4H)-one (0.5 g), 2-aminothiophenol (0.164 g), ethanol (10 ml) and toluene (5 ml) was stirred at 40° C. for 2 hr under a nitrogen atmosphere and then refluxed for 2 hr. The mixture was concentrated in vacuo. Water and saturated NaHCO₃ aqueous solution were added to the residue and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:1) and followed by recrystallization from ethyl acetate/hexane to give the title compound as crystals (0.2 g).

mp. 153-155° C.

¹H-NMR (300 MHz, DMSO-d₆) δ: 4.66 (2H, s), 5.86 (1H, s), 7.04 (1H, d, J=8.6 Hz), 7.10-7.19 (3H, m), 7.23-7.32 (4H, m), 7.46-7.52 (1H, m), 7.56 (1H, dd, J=8.6, 2.1 Hz), 7.79 (1H, d, J=2.1 Hz), 10.87 (1H, s).

### Example 40

### 6-[2-(3-Chlorophenyl)-2H-1,4-benzothiazin-3-yl]-2H-1,4-benzoxazin-3(4H)-one

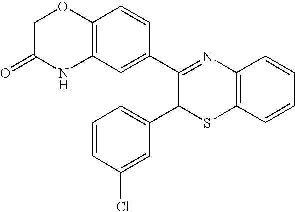

According to the similar procedure described for 6-[2-(4-chlorophenyl)-2H-1,4-benzothiazin-3-yl]-2H-1,4-benzoxazin-3(4H)-one, 6-[bromo(3-chlorophenyl)acetyl]-2H-1,4-benzoxazin-3(4H)-one (0.8 g) was reacted. The residue was crystallized from dichloromethane to give the title compound as crystals (0.4 g).

mp. 173-176° C.

¹H-NMR (300 MHz, DMSO-d₆) δ: 4.66 (2H, s), 5.89 (1H, s), 6.98-7.08 (2H, m), 7.11-7.34 (6H, m), 7.48-7.54 (1H, m), 7.57 (1H, dd, J=8.6, 2.1 Hz), 7.79 (1H, d, J=2.1 Hz), 10.87 (1H, s).

### Example 41

### 6-[2-(2-Chlorophenyl)-2H-1,4-benzothiazin-3-yl]-2H-1,4-benzoxazin-3(4H)-one

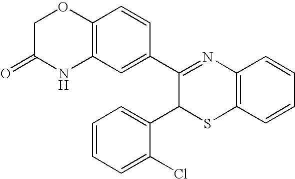

According to the similar procedure described for 6-[2-(4-chlorophenyl)-2H-1,4-benzothiazin-3-yl]-2H-1,4-benzoxazin-3(4H)-one, 6-[bromo(2-chlorophenyl)acetyl]-2H-1,4-benzoxazin-3(4H)-one (0.8 g) was reacted. The residue was crystallized from dichloromethane to give the title compound as crystals (0.35 g).

mp. 195-200° C.

¹H-NMR (300 MHz, DMSO-d₆) δ: 4.64 (2H, s), 5.73 (1H, s), 6.65 (1H, dd, J=7.7, 1.5 Hz), 6.99-7.36 (6H, m), 7.43 (1H, dd, J=8.6, 2.2 Hz), 7.53-7.62 (2H, m), 7.68 (1H, d, J=2.2 Hz), 10.88 (1H s).

### Example 42

### 6-(2-Benzyl-2H-1,4-benzothiazin-3-yl)-2H-1,4-benzoxazin-3(4H)-one

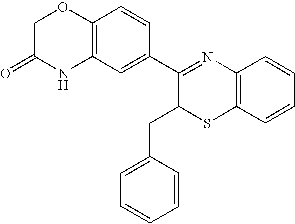

According to the similar procedure described for 6-[2-(4-chlorophenyl)-2H-1,4-benzothiazin-3-yl]-2H-1,4-benzoxazin-3(4H)-one, 6-(2-bromo-3-phenylpropanoyl)-2H-1,4-benzoxazin-3(4H)-one (0.4 g) was reacted. The residue was crystallized from dichloromethane to give the title compound as crystals (0.26 g).

mp. 193-194° C.

¹H-NMR (300 MHz, DMSO-d₆) δ: 2.44-2.56 (1H, m), 2.77 (1H, dd, J=13.7, 5.8 Hz), 4.58 (1H, dd, J=9.6, 5.8 Hz), 4.66 (2H, s), 6.99 (1H, d, J=8.6 Hz), 7.07-7.38 (7H, m), 7.43 (1H, dd, J=7.5, 1.3 Hz), 7.49 (1H, dd, J=7.8, 1.2 Hz), 7.56 (1H, dd, J=8.6, 2.1 Hz), 7.73 (1H, d, J=2.1 Hz), 10.86 (1H, s).

### Example 43

### 6-(2-Pyridin-2-yl-2H-1,4-benzothiazin-3-yl)-2H-1,4-benzoxazin-3(4H)-one

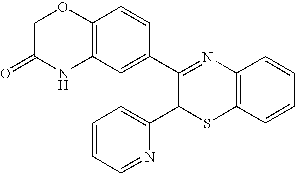

To a mixture of 6-[bromo(pyridin-2-yl)acetyl]-2H-1,4-benzoxazin-3(4H)-one hydrobromide (0.26 g), triethylamine (1 ml), ethanol (5 ml) and THF (10 ml) was added 2-aminothiophenol (0.12 g) at 80° C. and the mixture was stirred for 0.5 hr under a nitrogen atmosphere. 2-Aminothiophenol (0.12 g) was added to the mixture and the mixture was stirred for 4 hr at 80° C. The mixture was concentrated in vacuo. The organic layer was washed with water and brine, dried over Na₂SO₄ and concentrated in vacuo. The residue was purified by chromatography on silica gel (hexane→hexane:ethyl acetate=1:2) and followed by crystallization from methanol to give the title compound as crystals (42 mg).

mp. 168-170° C.

¹H-NMR (300 MHz, DMSO-d₆) δ: 4.65 (2H, s), 5.83 (1H, s), 7.02 (1H, d, J=8.7 Hz), 7.07-7.33 (5H, m), 7.42 (1H, dd, J=8.0, 1.1 Hz), 7.56-7.70 (2H, m), 7.79 (1H, d, J=2.3 Hz), 8.30-8.36 (1H, m), 10.84 (1H, s).

### Example 44

### 6-(2-Pyridin-2-yl-2H-thiochromen-3-yl)-2H-1,4-benzoxazin-3(4H)-one

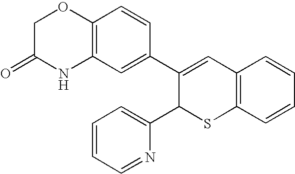

To a mixture of 2-mercaptobenzyltriphenylphosphonium bromide (0.2 g) in toluene (2 mL) was added 28% sodium methoxide in methanol (85 mg) at room temperature and the mixture was stirred at room temperature for 10 min. Then a mixture, which was prepared by addition of 28% sodium methoxide in methanol (85 mg) to a suspension of 6-[bromo(pyridin-2-yl)acetyl]-2H-1,4-benzoxazin-3(4H)-one hydrobromide (0.185 g) in a solution of THF (2 ml) and toluene (2 ml) at room temperature, was added and the mixture was stirred at 80° C. for 0.5 hr. 28% Sodium methoxide in methanol (170 mg) was added and the mixture was stirred at 80° C. for 4 hr. The mixture was concentrated in vacuo. Water and 10% hydrochloric acid were added to the residue and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:2) and followed by crystallization from methanol to give the title compound as crystals (26 mg).

mp. 220-213° C.

¹H-NMR (300 MHz, DMSO-d₆) δ: 4.56 (2H, s), 5.25 (1H, s), 6.93 (1H, d, J=8.33 Hz), 7.06 (1H, d, J=1.89 Hz), 7.09-7.27 (7H, m), 7.40-7.47 (1H, m), 7.58-7.67 (1H, m), 8.45-8.51 (1H, m), 10.71 (1H, s).

### Preparation 25

### 6-Pentanoyl-2H-1,4-benzoxazin-3(4H)-one

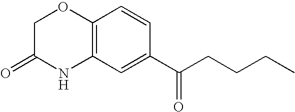

Aluminum chloride (20 g) was added to a suspension of 2H-1,4-benzoxazin-3(4H)-one (10 g) in 1,2-dichloroethane (120 ml) at room temperature and then valeryl chloride (9.6 ml) was added at room temperature. The reaction mixture was stirred at 80° C. for 3 hr, then poured into ice-cooled water. The mixture was extracted with dichloromethane. The organic layer was dried over Na₂SO₄ and concentrated in vacuo. The residue was crystallized from methanol to give the title compound as crystals (12.0 g).

¹H-NMR (300 MHz, DMSO-d₆) δ: 0.89 (3H, t, J=7.4 Hz), 1.24-1.40 (2H m), 1.50-1.64 (2H, m), 2.91 (2H, t, J=7.2 Hz), 4.68 (2H, s), 7.03 (1H, d, J=8.4 Hz), 7.48 (1H, d, J=2.0 Hz), 7.61 (1H, dd, J=8.4, 2.0 Hz), 10.85 (1H, s).

### Preparation 26

### 6-[(4-Chlorophenyl)acetyl]-2H-1,4-benzoxazin-3(4H)-one

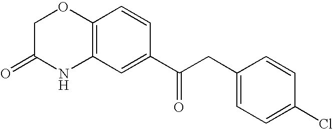

Aluminum chloride (15 g) was added to a suspension of 2H-1,4-benzoxazin-3(4H)-one (7.2 g) in 1,2-dichloroethane (90 ml) with ice-cooling and then 4-chlorophenylacetyl chloride (10.0 g) was added. The reaction mixture was allowed to warm to room temperature and stirred for 12 hr, then poured into ice-cooled water. 1,2-Dichloroethane layer was separated and the aqueous layer was extracted with ethyl acetate. 1,2-Dichloroethane layer was concentrated in vacuo and resulting residue was dissolved in ethyl acetate and combined with extracted ethyl acetate. Ethyl acetate layer was washed with water and brine, dried over Na₂SO₄ and concentrated in vacuo. The resulting crystals were washed with a solution of ethyl acetate and diisopropyl ether. The title compound was obtained as crystals (13.6 g).

¹H-NMR (300 MHz, DMSO-d₆) δ: 4.32 (2H, s), 4.69 (2H, s), 7.06 (1H, d, J=8.4 Hz), 7.23-7.31 (2H, m), 7.34-7.41 (2H, m), 7.51 (1H, d, J=2.1 Hz), 7.72 (1H, dd, J=8.4, 2.1 Hz), 10.88 (1H, s).

### Preparation 27

### 6-[(3-Chlorophenyl)acetyl]-2H-1,4-benzoxazin-3(4H)-one

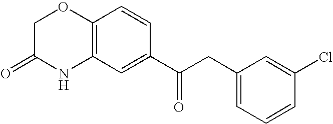

To a solution of 3-chlorophenylacetic acid (10.0 g) in THF (200 ml) was added DMF (5 drops) and then oxalyl chloride (8.0 ml) was added at room temperature, and the mixture was stirred for 1 hr. The mixture was concentrated in vacuo to give 3-chlorophenylacetyl chloride.

Aluminum chloride (16 g) was added to a suspension of 2H-1,4-benzoxazin-3(4H)-one (8.0 g) in 1,2-dichloroethane (100 ml) with ice-cooling and then 3-chlorophenylacetyl chloride obtained above was added. The reaction mixture was allowed to warm to room temperature and stirred for 12 hr, then poured into ice-cooled water (200 ml) and the resulting crystals were collected by filtration. The crystals were suspended in methanol (200 ml) and the mixture was refluxed for 2 hr. After cooling the mixture, the resulting crystals were collected by filtration. The title compound was obtained as crystals (14.9 g).

¹H-NMR (300 MHz, DMSO-d₆) δ: 4.35 (2H, s), 4.69 (2H, s), 7.07 (1H, d, J=8.3 Hz), 7.17-7.25 (1H, m), 7.26-7.40 (3H, m), 7.52 (1H, d, J=1.9 Hz), 7.73 (1H, dd, J=8.3, 1.9 Hz), 10.89 (1H, s).

### Preparation 28

### 6-[(2-Chlorophenyl)acetyl]-2H-1,4-benzoxazin-3(4H)-one

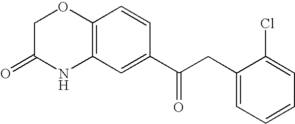

To a solution of 2-chlorophenylacetic acid (10.0 g) in THF (200 ml) was added DMF (5 drops) and then oxalyl chloride (8.0 ml) was added at room temperature, and the mixture was stirred for 1 hr. The mixture was concentrated in vacuo to give 2-chlorophenylacetyl chloride.

Aluminum chloride (16.0 g) was added to a suspension of 2H-1,4-benzoxazin-3(4H)-one (8.0 g) in 1,2-dichloroethane (100 ml) under ice-cooling and then 2-chlorophenylacetyl chloride obtained above was added. The reaction mixture was allowed to warm to room temperature and stirred for 12 hr, then poured into ice-cooled water (200 ml) and the resulting crystals were collected by filtration. The crystals were suspended in methanol (200 ml) and the mixture was refluxed for 2 hr. After cooling the mixture, the resulting crystals were collected by filtration. The title compound was obtained as crystals (13.3 g).

¹H-NMR (300 MHz, DMSO-d₆) δ: 4.47 (2H, s), 4.70 (2H, s), 7.09 (1H, d, J=8.3 Hz), 7.27-7.50 (4H, m), 7.54 (1H, d, J=2.1 Hz), 7.76 (1H, dd, J=8.3, 2.1 Hz), 10.89 (1H, s).

### Preparation 29

### 6-[(4-Fluorophenyl)acetyl]-2H-1,4-benzoxazin-3(4H)-one

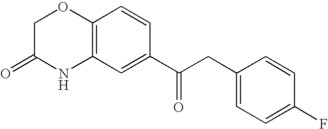

To a solution of 4-fluorophenylacetic acid (9.9 g) in THF (100 ml) was added DMF (5 drops) and then oxalyl chloride (9.0 ml) was added at room temperature, and the mixture was stirred for 1 hr. The mixture was concentrated in vacuo to give 4-fluorophenylacetyl chloride. Aluminum chloride (16.0 g) was added to a suspension of 2H-1,4-benzoxazin-3(4H)-one (8.0 g) in 1,2-dichloroethane (100 ml) under ice-cooling and then 4-fluorophenylacetyl chloride obtained above was added. The reaction mixture was allowed to warm to room temperature and stirred for 12 hr, then poured into ice-cooled water (200 ml) and the resulting crystals were collected by filtration. The crystals were suspended in methanol and the mixture was refluxed for 1 hr. After cooling the mixture, the resulting crystals were collected by filtration. The title compound was obtained as crystals (5.45 g).

### Preparation 30

### 6-(Pyridin-2-ylacetyl)-2H-1,4-benzoxazin-3(4H)-one

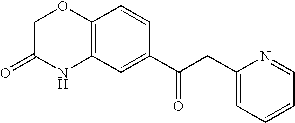

To a mixture of 2H-1,4-benzoxazin-3(4H)-one (5.0 g) and polyphosphoric acid (150 g) was added 2-pyridylacetic acid hydrochloride (8.7 g) at 80° C. and the mixture was stirred for 0.5 hr. The mixture was allowed to warm to 130° C. and stirred for 24 hr. The mixture was added to ice-cooled water (300 ml). The aqueous mixture was filtered and the filtrate was adjusted to pH 8 by the addition of 8N-NaOH. The mixture was stirred at 60° C. for 2 hr and then cooled to 50° C. The resulting crystals were collected by filtration and washed with water. The crystals were suspended in methanol and the mixture was refluxed for 1 hr. After cooling the mixture to room temperature, the resulting crystals were collected. The title compound was obtained as crystals (4.0 g).

Anal. Calcd for C₁₅H₁₂N₂O₃: C, 67.16; H, 4.51; N, 10.44.

Found: 67.87; H, 4.46; N, 10.39.

### Preparation 31

### 6-[3-Phenylprop-2-enoyl]-2H-1,4-benzoxazin-3(4H)-one

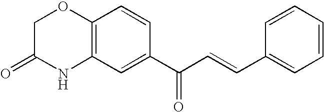

To a mixture of 6-acetyl-2H-1,4-benzoxazin-3(4H)-one (4 g) and benzaldehyde (2.7 g) in methanol (40 ml) was added 28% sodium methoxide in methanol (4.4 g) at room temperature and the mixture was stirred at 50° C. for 24 hr. The mixture was concentrated in vacuo and then water and 10% hydrochloric acid were added to the residue. The resulting crystals were collected by filtration and then suspended in methanol (40 ml). The mixture was refluxed for 0.5 hr and then cooled to room temperature. The resulting crystals were collected by filtration. The title compound was obtained as crystals (5.07 g).

¹H-NMR (300 MHz, DMSO-d₆) δ: 4.72 (2H, s), 7.10 (1H, d, J=8.7 Hz), 7.41-7.52 (3H, m), 7.62 (1H, d, J=1.9 Hz), 7.71 (1H, d, J=15.5 Hz), 7.81-7.96 (4H, m), 10.89 (1H, s).

### Preparation 32

### 6-(3-Phenylpropanoyl)-2H-1,4-benzoxazin-3(4H)-one

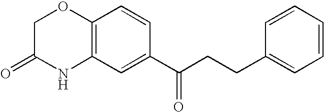

A mixture of 6-[3-phenylprop-2-enoyl]-2H-1,4-benzoxazin-3(4H)-one (4.0 g), 10% palladium-carbon (2.0 g), ethanol (80 ml) and THF (80 ml) was stirred under an hydrogen atmosphere (1 atm) at room temperature for 2 hr. The catalyst was filtered off and the filtrate was concentrated in vacuo. The residue was crystallized from methanol. The title compound was obtained as crystals (1.0 g).

¹H-NMR (300 MHz, DMSO-d₆) δ: 2.91 (2H, t, J=7.6 Hz), 3.27 (2H, t, J=7.6 Hz), 4.68 (2H, s), 7.03 (1H, d, J=8.3 Hz), 7.12-7.33 (5H, m), 7.49 (1H, d, J=2.1 Hz), 7.63 (1H, dd, J=8.3, 2.1 Hz), 10.84 (1H, s).

### Preparation 33

### 6-(2-Phenylacryloyl)-2H-1,4-benzoxazin-3(4H)-one

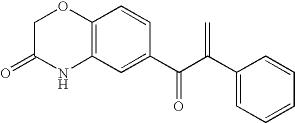

To a mixture of 6-(phenylacetyl)-2H-1,4-benzoxazin-3(4H)-one (7.0 g), N,N,N′,N′-tetramethyldiaminomethane (10.5 ml) and dichloromethane (14 ml) was added acetic anhydride (10.5 ml) with ice-cooling and the mixture was allowed to warm to room temperature. After stirring for 72 hr at room temperature, the mixture was concentrated in vacuo. Ethyl acetate and water was added to the residue and then the organic layer was separated. The organic layer was washed with brine, dried over Na₂SO₄ and concentrated in vacuo. The resulting crystals were suspended in methanol (70 ml) and the mixture was stirred at 45° C. for 1 hr. After cooling the mixture to room temperature, the resulting crystals were collected by filtration. The title compound was obtained as crystals (5.75 g).

¹H-NMR (300 MHz, DMSO-d₆) δ: 4.69 (2H, s), 5.52 (1H, s), 6.15 (1H, s), 7.04 (1H, d, J=8.3 Hz), 7.28-7.52 (7H, m), 10.89 (1H, s).

### Preparation 34

### 6-(2-Phenylpropanoyl)-2H-1,4-benzoxazin-3(4H)-one

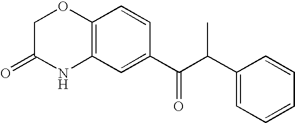

A mixture of 6-(2-phenylacryloyl)-2H-1,4-benzoxazin-3(4H)-one (3.0 g), 10% palladium-carbon (1.0 g) and THF (60 ml) was stirred under an hydrogen atmosphere (1 atm) at room temperature for 1 hr. The catalyst was filtered off and the filtrate was concentrated in vacuo. The residue was purified by chromatography on basic silica gel (hexane→hexane:ethyl acetate=1:1) to give the title compound as crystals (1.98 g).

¹H-NMR (300 MHz, DMSO-d₆) δ: 1.38 (3H, d, J=6.8 Hz), 4.63 (2H, s), 4.81 (1H, q, J=6.8 Hz), 6.96 (1H, d, J=8.6 Hz), 7.13-7.34 (5H, m), 7.50 (1H, d, J=2.2 Hz), 7.64 (1H, dd, J=8.6, 2.2 Hz), 10.84 (1H, s).

### Preparation 35

### 6-(2-Bromopentanoyl)-2H-1,4-benzoxazin-3(4H)-one

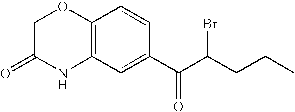

To a suspension of 6-pentanoyl-2H-1,4-benzoxazin-3(4H)-one (10 g) in acetic acid (80 ml) was added 25% hydrogen bromide in acetic acid (20 ml) at room temperature and then pyridinium hydrobromide perbromide (14.4 g) was added portionwise to the mixture at room temperature. After stirring the mixture for 2 hr, water (300 ml) was added dropwise to the mixture at room temperature. The resulting crystals were collected by filtration. The title compound was obtained as crystals (13.1 g).

¹H-NMR (300 MHz, CDCl₃) δ: 0.99 (3H, t, J=7.4 Hz), 1.32-1.70 (2H, m), 2.01-2.26 (2H, m), 4.73 (2H, s), 5.07 (1H, dd, J=7.6, 6.7 Hz), 7.04 (1H, d, J=8.5 Hz), 7.56 (1H, d, J=2.0 Hz), 7.67 (1H, dd, J=8.5, 2.0 Hz), 8.56 (1H, s).

### Preparation 36

### 6-[Bromo(4-chlorophenyl)acetyl]-2H-1,4-benzoxazin-3(4H)-one

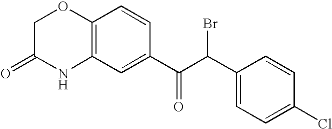

To a suspension of 6-[(4-chlorophenyl)acetyl]-2H-1,4-benzoxazin-3(4H)-one (13.0 g) in acetic acid (120 ml) was added 25% hydrogen bromide in acetic acid (30 ml) at room temperature and then pyridinium hydrobromide perbromide (14.5 g) was added in portionwise to the mixture at room temperature. After stirring the mixture for 15 min, aqueous sodium sulfite solution, which was prepared from sodium sulfite (1.1 g) and water (100 ml), was added dropwise to the mixture with ice-cooling and then water (200 ml) was added dropwise with ice-cooling. The resulting crystals were collected by filtration and washed with water. Then obtained crystals were suspended in methanol (60 ml) and the mixture was stirred for 1 hr at room temperature. The crystals were collected by filtration. The title compound was obtained as crystals (16.1 g).

¹H-NMR (300 MHz, DMSO-d₆) δ: 4.69 (2H, s), 7.06 (1H, d, J=8.6 Hz), 7.08 (1H, s), 7.46 (2H, d, J=8.7 Hz), 7.53 (1H, d, J=2.1 Hz), 7.57 (2H, d, J=8.7 Hz), 7.77 (1H, dd, J=8.6, 2.1 Hz), 10.93 (1H, s).

### Preparation 37

### 6-[Bromo(3-chlorophenyl)acetyl]-2H-1,4-benzoxazin-3(4H)-one

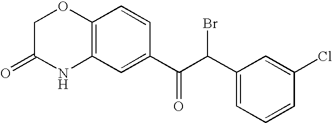

To a suspension of 6-[(3-chlorophenyl)acetyl]-2H-1,4-benzoxazin-3(4H)-one (10.0 g) in acetic acid (100 ml) was added 25% hydrogen bromide in acetic acid (25 ml) at room temperature and then pyridinium hydrobromide perbromide (11.1 g) was added portionwise to the mixture at room temperature. After stirring the mixture for 15 min, aqueous sodium sulfite solution, which was prepared from sodium sulfite (0.83 g) and water (50 ml), was added dropwise to the mixture with ice-cooling and then water (250 ml) was added dropwise with ice-cooling. The resulting crystals were collected by filtration and washed with water. Then obtained crystals were suspended in methanol (50 ml) and the mixture was stirred for 1 hr at room temperature. The crystals were collected by filtration. The title compound was obtained as crystals (11.6 g).

¹H-NMR (300 MHz, DMSO-d₆) δ: 4.70 (2H, s), 7.05 (1H, s), 7.08 (1H, d, J=8.6 Hz), 7.37-7.67 (5H, m), 7.79 (1H, dd, J=8.6, 2.1 Hz), 10.94 (1H, s).

### Preparation 38

### 6-[Bromo(2-chlorophenyl)acetyl]-2H-1,4-benzoxazin-3(4H)-one

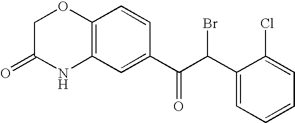

To a suspension of 6-[(2-chlorophenyl)acetyl]-2H-1,4-benzoxazin-3(4H)-one (8.0 g) in acetic acid (80 ml) was added 25% hydrogen bromide in acetic acid (20 ml) at room temperature and then pyridinium hydrobromide perbromide (8.9 g) was added portionwise to the mixture at room temperature. After stirring the mixture for 15 min, aqueous sodium sulfite solution, which was prepared from sodium sulfite (0.7 g) and water (40 ml), was added dropwise to the mixture under ice-cooling and then water (200 ml) was added dropwise under ice-cooling. The resulting crystals were collected by filtration and washed with water. Then obtained crystals were suspended in methanol (40 ml) and the mixture was stirred for 1 hr at room temperature. The crystals were collected by filtration. The title compound was obtained as crystals (9.16 g).

¹H-NMR (300 MHz, DMSO-d₆) δ: 4.68 (2H, s), 7.04 (1H, d, J=8.4 Hz), 7.15 (1H, s), 7.33-7.57 (5H, m), 7.60 (1H, dd, J=8.4, 2.1 Hz), 10.93 (1H, s).

### Preparation 39

### 6-[Bromo(4-fluorophenyl)acetyl]-2H-1,4-benzoxazin-3(4H)-one

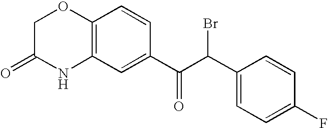

To a suspension of 6-[(4-fluorophenyl)acetyl]-2H-1,4-benzoxazin-3(4H)-one (2.4 g) in acetic acid (20 ml) was added 25% hydrogen bromide in acetic acid (5 ml) at room temperature and then pyridinium hydrobromide perbromide (2.8 g) was added portionwise to the mixture at room temperature. After stirring the mixture for 15 min, aqueous sodium sulfite solution, which was prepared from sodium sulfite (0.32 g) and water (10 ml), was added dropwise to the mixture under ice-cooling and then water (40 ml) was added dropwise under ice-cooling. The resulting crystals were collected by filtration and washed with water. The title compound was obtained as crystals (2.94 g).

¹H-NMR (300 MHz, DMSO-d₆) δ: 4.69 (2H, s), 7.01-7.12 (2H, m), 7.23 (2H, t, J=8.90 Hz), 7.54 (1H, d, J=2.0 Hz), 7.56-7.65 (2H, m), 7.78 (1H, dd, J=8.5, 2.0 Hz), 10.93 (1H, s).

### Preparation 40

### 6-(2-Bromo-3-phenylpropanoyl)-2H-1,4-benzoxazin-3(4H)-one

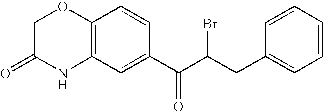

The title compound was obtained as crystals (1.1 g) from 6-(3-phenylpropanoyl)-2H-1,4-benzoxazin-3(4H)-one (0.9 g) according to a method similar to the procedure for 6-[bromo(3-chlorophenyl)acetyl]-2H-1,4-benzoxazin-3(4H)-one.

¹H-NMR (300 MHz, DMSO-d₆) δ: 3.23 (1H, dd, J=14.3, 7.3 Hz), 3.52 (1H, dd, J=14.3, 7.3 Hz), 4.70 (2H, s), 5.82 (1H, t, J=7.3 Hz), 7.05 (1H, d, J=8.4 Hz), 7.16-7.39 (5H, m), 7.52 (1H, d, J=2.0 Hz), 7.74 (1H, dd, J=8.4, 2.0 Hz), 10.87 (1H, s).

### Preparation 41

### 6-(2-Bromo-2-phenylpropanoyl)-2H-1,4-benzoxazin-3(4H)-one

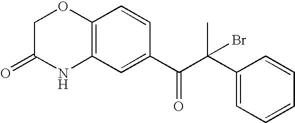

The title compound was obtained as crystals (2.24 g) from 6-(2-phenylpropanoyl)-2H-1,4-benzoxazin-3(4H)-one (1.8 g) according to a method similar to the procedure for 6-[bromo(3-chlorophenyl)acetyl]-2H-1,4-benzoxazin-3(4H)-one.

¹H-NMR (300 MHz, DMSO-d₆) δ: 2.14 (3H, s), 4.63 (2H, s), 6.82 (1H, d, J=8.4 Hz), 7.15 (1H, dd, J=8.4, 2.1 Hz), 7.29-7.50 (6H, m), 10.88 (1H, s).

### Preparation 42

### 6-[Bromo(pyridin-2-yl)acetyl]-2H-1,4-benzoxazin-3(4H)-one hydrobromide

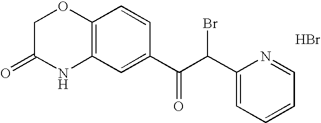

To a solution of 6-(pyridin-2-ylacetyl)-2H-1,4-benzoxazin-3(4H)-one (1.0 g) in acetic acid (8 ml) was added a solution of bromine (0.21 ml) in acetic acid (2 ml) dropwise at room temperature and the mixture was stirred for 1 hr at room temperature. Bromine (0.04 ml) was added to the mixture at room temperature and the mixture was stirred for 1 hr. The mixture was concentrated in vacuo and 25% hydrogen bromide in acetic acid was added to the residue. The mixture was concentrated in vacuo. The residue was crystallized from methanol to give the title compound as crystals (1.11 g).

¹H-NMR (300 MHz, DMSO-d₆) δ: 4.67 (2H, s), 6.99 (1H, d, J=8.2 Hz), 7.12 (1H, s), 7.34-7.42 (1H, m), 7.50 (1H, d, J=2.0 Hz), 7.62 (1H, dd, J=8.2, 2.0 Hz), 7.74 (1H, d, J=8.0 Hz), 7.88-7.99 (1H, m), 8.48-8.55 (1H, m), 10.90 (1H, s), 1H was unconfirmed.

### Example 45

### (S)-6-(2-Phenyl-2H-thiochromen-3-yl)-2H-1,4-benzoxazin-3(4H)-one

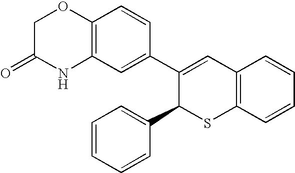

Separation of 6-(2-Phenyl-2H-thiochromen-3-yl)-2H-1,4-benzoxazin-3(4H)-one was carried out by HPLC using Kromasil 5CHI DMB (30 mm i.d.×250 mm) with detection at 254 nm. Elution with a mixture of n-hexane/ethyl acetate (50/50) at a flow rate of 20 mL/min at room temperature gave the title compound: retention time=32.8 min. Stereochemistry was assigned by single-crystal X-ray analysis.

¹H-NMR (DMSO-d₆) δ: 4.56 (2H, s), 5.30 (1H, s), 6.92 (1H, d, J=8.7 Hz), 7.06-7.27 (11H, m), 7.43 (1H, d, J=6.6 Hz), 10.70 (1H, brs).

### Example 46

### 6-(1,4-Diphenyl-4,5-dihydro-1H-pyrazol-3-yl)-2H-benzo[b][1,4]oxazin-3(4H)-one

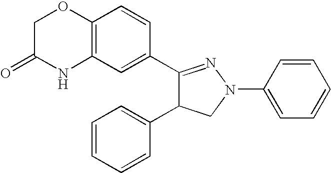

### 6-(2-Phenylacryloyl)-2H-benzo[b][1,4]oxazin-3(4H)-one

To a solution of 6-(2-phenylacetyl)-2H-benzo[b][1,4]oxazin-3(4H)-one (7.32 g, 27.4 mmol) and N,N,N′,N′-tetramethyldiaminomethane (7.30 mL, 54.8 mmol) in THF (100 mL) was added acetic anhydride (7.0 mL, 74.2 mmol) with stirring at 0° C. After stirring for 30 min at 0° C., the mixture was allowed to warm to room temperature for 3 hr, and then warmed to 50° C. for 1 hr. The reaction mixture was diluted with ice-water, half of the THF was removed in vacuo (without heating) and the mixture was filtered. The solid was washed with water and dried under vacuum to give the title compound as a cream colored solid (6.9 g, 90%).

¹H-NMR (400 MHz, CDCl₃) δ: 7.94 (s, 1H), 7.55 (dd, J=8.6, 2.0 Hz, 1H), 7.44 (d, J=2.0 Hz, 1H), 7.41-7.32 (m, 5H), 6.97 (d, J=8.6 Hz, 1H), 6.03 (s, 1H), 5.60 (s, 1H), 4.69 (s, 2H); LCMS (ESI⁺), M+H⁺: 280 (100%).

### 6-(1,4-Diphenyl-4,5-dihydro-1H-pyrazol-3-yl)-2H-benzo[b][1,4]oxazin-3(4H)-one

To a solution of 6-(2-phenylacryloyl)-2H-benzo[b][1,4]oxazin-3(4H)-one (500 mg, 1.79 mmol) in degassed MeOH (10 mL) was added 1-phenylhydrazine (352 μL, 3.60 mmol) at room temperature. The mixture was stirred for 1 hr at room temperature and then warmed to 40° C. for 1 hr, or until the starting material was consumed. The mixture was cooled, poured into ice-water and filtered. The solid was washed with water, dried, and further purified by flash chromatography on silica gel (0-12% EtOAc in DCM) to give the title compound as a pale yellow solid (260 mg, 39%).

¹H-NMR (400 MHz, CDCl₃) δ: 7.58 (bs, 1H), 7.31 (m, 5H), 7.25 (m, 1H), 7.23 (m, 1H), 7.21 (d, J=1.6 Hz, 1H), 7.14 (m, 3H), 6.88 (t, J=7.4 Hz, 1H), 6.83 (d, J=8.6 Hz, 1H), 4.66 (dd, J=11.3, 5.5 Hz, 1H), 4.58 (s, 2H), 4.23 (dd, J=11.3, 10.2 Hz, 1H) 3.91 (dd, J=10.2, 5.5 Hz, 1H); LCMS (APCI⁺), M+H⁺: 370.

### Example 47

### 6-(4-Phenyl-4,5-dihydro-1H-pyrazol-3-yl)-2H-benzo[b][1,4]oxazin-3(4H)-one

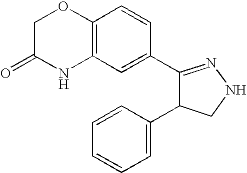

According to the method of Example 46, 6-(2-phenylacryloyl)-2H-benzo[b][1,4]oxazin-3(4H)-one (200 mg, 0.72 mmol) and hydrazine (130 μL, 1.43 mmol) were reacted to give the title compound as a white solid (180 mg, 85%).

¹H-NMR (400 MHz, CDCl₃) δ: 7.65 (bs, H), 7.23-7.32 (m, 4H), 7.16 (d, J=1.6 Hz, 1H), 7.06 (dd, J=8.6, 1.6 Hz, 1H), 6.82 (d, J=8.6 Hz, 1H), 5.80 (s, 1H), 4.58 (s, 2H), 4.47 (dd, J=10.5, 5.1 Hz, 1H), 3.97 (m, 1H), 3.54 (dd, J=9.4, 5.1 Hz, 1H); LCMS (ESI⁺), M+H⁺: 294.

### Example 48

### 6-(1-Methyl-4-phenyl-4,5-dihydro-1H-pyrazol-3-yl)-2H-benzo[b][1,4]oxazin-3(4H)-one

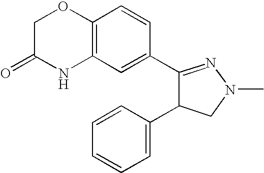

According to the method of Example 46, 6-(2-phenylacryloyl)-2H-benzo[b][1,4]oxazin-3(4H)-one (100 mg, 0.36 mmol) and 1-methylhydrazine (33 mg, 0.72 mmol) were reacted to give the title compound as an off-white solid (40 mg, 36%).

¹H-NMR (400 MHz, CDCl₃) δ: 7.29 (m, 2H), 7.23 (m, 3H), 7.13 (d, J=2.0 Hz, 1H), 7.01 (dd, J=8.2, 1.6 Hz, 1H), 6.80 (d, J=8.2 Hz, 1H), 4.56 (s, 2H), 4.47 (dd, J=10.2, 5.1 Hz, 1H), 3.43 (m, 1H), 3.34 (dd, J=9.4, 5.1 Hz, 1H), 2.95 (s, 3H); LCMS (ESI⁺), M+H⁺: 308.

### Example 49

### 6-(1-(4-Fluorophenyl)-4-phenyl-4,5-dihydro-1H-pyrazol-3-yl)-2H-benzo[b][1,4]oxazin-3(4H)-one

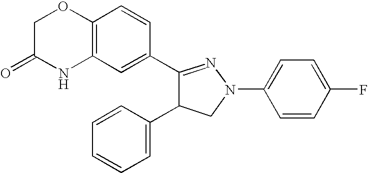

According to the method of Example 46, 6-(2-phenylacryloyl)-2H-benzo[b][1,4]oxazin-3(4H)-one (200 mg, mmol), 1-(4-fluorophenyl)hydrazine hydrochloride (175 mg, 1.07 mmol) and triethylamine (160 μL, 1.15 mmol) were reacted in ethanol at 60° C. to give the title compound as a pale yellow solid (60 mg, 21%).

¹H-NMR (400 MHz, CDCl₃) δ: 7.71 (bs, 1H), 7.31 (m, 2H), 7.23 (s, 1H), 7.20 (s, 1H), 7.07-7.14 (m, 4H), 7.01 (t, 3H), 6.84 (d, J=8.6 Hz, 1H), 4.66 (dd, J=11.7, 5.3 Hz, 1H), (s, 2H), 4.17 (m, 1H) 3.87 (dd, J=9.7, 5.3 Hz, 1H);

LCMS (APCI⁺), M+H⁺: 388.

### Example 50

### 6-(1-(3,4-Dichlorophenyl)-4-phenyl-4,5-dihydro-1H-pyrazol-3-yl)-2H-benzo[b][1,4]oxazin-3(4H)-one

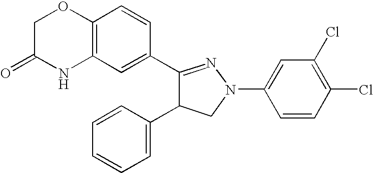

To a solution of 6-(2-phenylacryloyl)-2H-benzo[b][1,4]oxazin-3(4H)-one (200 mg, 0.72 mmol) in DMF mL) was added 1-(3,4-dichlorophenyl)hydrazine hydrochloride (229 mg, 1.07 mmol) followed by triethylamine (300 μL, 2.15 mmol), and the mixture was heated at 50° C. for 12 hr. The reaction mixture was diluted with EtOAc, washed with 1N HCl, brine, saturated aqueous NaHCO₃ and brine, dried (Na₂SO₄) and concentrated in vacuo. Flash chromatography on silica gel (0-20% EtOAc in DCM) followed by preparative TLC gave the title compound as a pale yellow powder (10 mg, 3%).

¹H-NMR (400 MHz, CDCl₃) δ: 7.76 (bs, 1H), 7.32 (m, 3H), 7.24-7.28 (m, 2H), 7.21 (m, 3H), 7.14 (dd, J=8.6, 2.3 Hz, 1H), 6.92 (dd, J=8.6, 2.3, 1H), 6.84 (d, J=8.6 Hz 1H), 4.70 (dd, J=11.4, 5.2 Hz, 1H), 4.60 (s, 2H), 4.19 (dd, J=11.4, 10.0 Hz, 1H) 3.86 (dd, J=10.0, 5.2 Hz, 1H); LCMS (ESI⁻), M−H⁻: 436.

### Example 51

### 6-(1-(4-Chlorophenyl)-4-phenyl-4,5-dihydro-1H-pyrazol-3-yl)-2H-benzo[b][1,4]oxazin-3(4H)-one

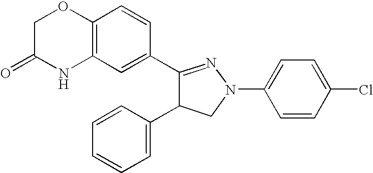

According to the method of Example 50, 6-(2-phenylacryloyl)-2H-benzo[b][1,4]oxazin-3(4H)-one (200 mg, 0.72 mmol), 1-(4-chlorophenyl)hydrazine hydrochloride (192 mg, 1.07 mmol) and triethylamine (160 μL, 1.15 mmol) were reacted in THF at 60° C. to give the title compound as a pale yellow solid (50 mg, 17%).

¹H-NMR (400 MHz, CDCl₃) δ: 7.83 (bs, 1H), 7.31 (m, 2H), 7.22-7.27 (m, 5H), 7.20 (d, J=2.0 Hz, 1H), 7.13 (dd, J=8.0, 2.0 Hz, 1H), 7.06 (d, J=9.0 Hz, 2H), 6.83 (d, J=8.0 Hz, 1H), 4.68 (dd, J=11.5, 5.3 Hz, 1H), 4.59 (s, 2H), 4.19 (dd, J=11.5, 10.0 Hz, 1H) 3.88 (dd, J=10.0, 5.2 Hz, 1H); LCMS (APCI⁺), M+H⁺: 404.

### Example 52

### 6-(1,4-Bis(4-fluorophenyl)-4,5-dihydro-1H-pyrazol-3-yl)-2H-benzo[b][1,4]oxazin-3(4H)-one

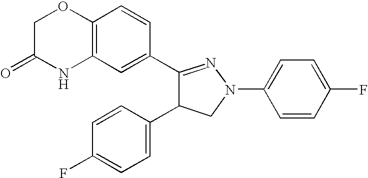

### 6-(2-(4-Fluorophenyl)acetyl)-2H-benzo[b][1,4]oxazin-3(4H)-one

According to method of Preparation 13, 2H-benzo[b][1,4]oxazin-3(4H)-one (10.0 g, 67.05 mmol) and 4-fluorophenylacetyl chloride (11.0 mL, 80.5 mmol) were reacted to give the title compound as an off-white solid (18.0 g, 94%).

¹H-NMR (400 MHz, CDCl₃) δ: 8.66 (bs, 1H), 7.66 (dd, J=8.6, 2.0 Hz, 1H), 7.52 (d, J=2.0 Hz, 1H), 7.21 (m, 2H), 7.01 (m, 3H), 4.70 (s, 2H), 4.20 (s, 2H); LCMS (ESI⁻), M−H⁻: 284.

### 6-(2-(4-Fluorophenyl)acryloyl)-2H-benzo[b][1,4]oxazin-3(4H)-one

According to the method of Example 46, 6-(2-(4-fluorophenyl)acetyl)-2H-benzo[b][1,4]oxazin-3(4H)-one (9.54 g, 33.4 mmol) was reacted to give the title compound as a white solid (9.50 g, 95%).

¹H-NMR (400 MHz, CDCl₃) δ: 8.34 (bs, 1H), 7.53 (dd, J=8.6, 2.0 Hz, 1H), 7.46 (d, J=2.0 Hz, 1H), 7.04 (m, 2H), 6.98 (d, J=8.6 Hz, 1H), 6.00 (s, 1H), 5.59 (s, 1H), 4.70 (s, 2H); LCMS (ESI⁻), M−H⁻: 296.

### 6-(1,4-Bis(4-fluorophenyl)-4,5-dihydro-1H-pyrazol-3-yl)-2H-benzo[b][1,4]oxazin-3(4H)-one

According to the method of Example 46, 6-(2-(4-fluorophenyl)acryloyl)-2H-benzo[b][1,4]oxazin-3(4H)-one (300 mg, 1.01 mmol), 1-(4-fluorophenyl)hydrazine hydrochloride (328 mg, 2.02 mmol) and triethylamine (280 μL, 2.02 mmol) were reacted to give the title compound as a pale yellow powder (120 mg, 29%).

¹H-NMR (400 MHz, CDCl₃) δ: 8.30 (bs, 1H), 7.21 (m, 3H), 7.09 (m, 3H), 7.00 (m, 4H), 6.85 (d, J=8.2 Hz, 1H), 4.65 (dd, J=11.5, 4.9 Hz, 1H), 4.61 (s, 2H), 4.13 (m, 1H) 3.85 (dd, J=9.6, 4.9 Hz, 1H); LCMS (APCI⁺), M+H⁺ 406.

### Example 53

### 6-(4-Phenyl-1-(2,2,2-trifluoroethyl)-4,5-dihydro-1H-pyrazol-3-yl)-2H-benzo[b][1,4]oxazin-3(4H)-one

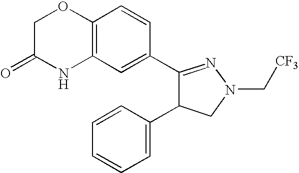

According to the method of Example 50, 6-(2-phenylacryloyl)-2H-benzo[b][1,4]oxazin-3(4H)-one (200 mg, 0.72 mmol) and 1-(2,2,2-trifluoroethyl)hydrazine (175 mg, 1.07 mmol) were reacted in methanol to give the title compound as a white solid (160 mg, 59%).

¹H-NMR (400 MHz, CDCl₃) δ: 7.84 (bs, 1H), 7.23-7.32 (m, 5H), 7.10 (d, J=2.0 Hz, 1H), 7.04 (dd, J=8.4, 2.0 Hz, 1H), 6.81 (d, J=8.4 Hz, 1H), 4.58 (s, 2H), 4.51 (dd, J=10.5, 5.5 Hz, 1H), 3.98 (m, 1H), 3.65 (m, 2H) 3.51 (dd, J=9.4, 5.5 Hz, 1H); LCMS (ESI⁻), M−H⁻: 374.

6-(4-Phenyl-1-(2,2,2-trifluoroethyl)-1H-pyrazol-3-yl)-2H-benzo[b][1,4]oxazin-3(4H)-one (30 mg, 11%) was also obtained as a white solid.

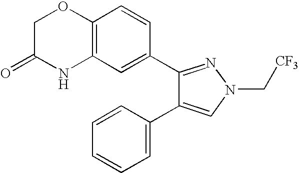

¹H-NMR (400 MHz, CDCl₃) δ: 8.89 (bs, 1H), 7.85 (s, 1H), 7.24 (m, 2H), 7.18 (m, 3H), 7.07 (d, J=8.2 Hz, 1H), 6.94 (dd, J=8.4, 2.0 Hz, 1H), 6.75 (d, J=2.0 Hz, 1H), 4.69 (s, 2H) 4.57 (q, J=8.2 Hz, 2H); LCMS (ESI⁺), M+H⁺: 374.

### Example 54

### 6-(1-Benzyl-4-phenyl-4,5-dihydro-1H-pyrazol-3-yl)-2H-benzo[b][1,4]oxazin-3(4H)-one

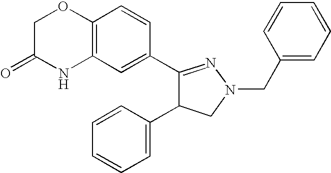

According to the method of Example 46, 6-(2-phenylacryloyl)-2H-benzo[b][1,4]oxazin-3(4H)-one (200 mg, 0.72 mmol), 1-benzylhydrazine dihydrochloride (140 mg, 0.72 mmol) and pyridine (120 μL, 1.43 mmol) were reacted to give the title compound as a white solid (80 mg, 29%).

¹H-NMR (400 MHz, DMSO-d₆) δ: 10.67 (bs, 1H), 7.32-7.40 (m, 5H), 7.21-7.29 (m, 5H), 7.17 (m, 1H), 6.97 (dd, J=8.6, 2.0 Hz, 1H), 6.82 (d, J=8.2 Hz, 1H), 4.57 (dd, J=10.5, 3.7 Hz, 1H), 4.53 (s, 2H), 4.43 (d, J=13.7 Hz, 1H), 4.17 (d, J=13.7 Hz, 1H) 3.36 (m, 1H), 3.17 (dd, J=9.8, 3.7 Hz, 1H); LCMS (ESI⁺), M+H⁺: 384.

### Example 55

### 6-(1-Butyl-4-phenyl-4,5-dihydro-1H-pyrazol-3-yl)-2H-benzo[b][1,4]oxazin-3(4H)-one

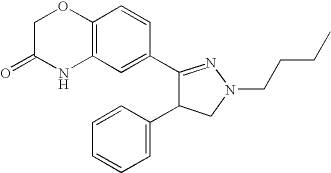

According to the method of Example 46, 6-(2-phenylacryloyl)-2H-benzo[b][1,4]oxazin-3(4H)-one (200 mg, 0.72 mmol), butylhydrazine dihydrochloride (140 mg, 0.72 mmol) and pyridine (120 μL, 1.43 mmol) were reacted in CHCl₃ to give the title compound as a white solid (40 mg, 16%).

¹H-NMR (400 MHz, CDCl₃) δ: 8.82 (bs, 1H), 7.19-7.27 (m, 6H), 7.01 (dd, J=8.6, 2.0 Hz, 1H), 6.77 (d, J=8.6 Hz, 1H), 4.54 (s, 2H), 4.43 (dd, J=10.2, 5.5 Hz, 1H), 3.48 (t, 1H), 3.34 (dd, J=9.8, 5.5 Hz, 1H) 3.24 (ddd, J=12.1, 8.4, 6.8 Hz, 1H), 3.01 (ddd, J=12.1, 8.2, 6.4 Hz, 1H), 1.66 (m, 2H), 1.44 (m, 2H), 0.95 (t, J=7.2 Hz, 3H); LCMS (APCI⁺), M+H⁺: 350.

### Example 56

### 6-(4-Phenyl-1H-pyrazol-3-yl)-2H-benzo[b][1,4]oxazin-3(4H)-one

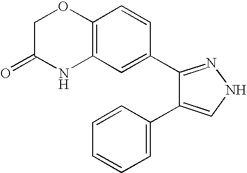

To a solution of 6-(4-phenyl-4,5-dihydro-1H-pyrazol-3-yl)-2H-benzo[b][1,4]oxazin-3(4H)-one (30 mg, 0.10 mmol) in THF (2.0 mL) were added trifluoromethanesulfonyl chloride (10.9 μL, 0.10 mmol), pyridine (8.27 μL, 0.10 mmol) and DMAP (1.25 mg, 0.01 mmol) at room temperature and the mixture was stirred overnight. After dilution with EtOAc, the mixture was washed with 1N HCl, brine, 0.5N NaOH and brine, dried (Na₂SO₄) and concentrated in vacuo. No pyrazoline sulfonamide was observed. After preparative TLC (5% MeOH in DCM), the title compound was obtained as an off-white powder (6 mg, 20%).

¹H-NMR (400 MHz, CDCl₃) δ: 10.46 (bs, 1H), 7.70 (s, 1H), 7.50 (s, 1H), 7.33 (m, 6H), 6.91 (dd, J=8.6, 2.0 Hz, 1H), 6.83 (d, J=8.6 Hz, 1H), 4.70 (s, 2H); LCMS (APCI⁺), M+H⁺: 292.

### Example 57

### 6-(4-Phenyl-1-(2,2,2-trifluoroacetyl)-4,5-dihydro-1H-pyrazol-3-yl)-2H-benzo[b][1,4]oxazin-3(4H)-one

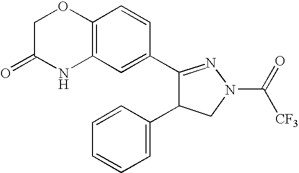

To a solution of 6-(4-phenyl-4,5-dihydro-1H-pyrazol-3-yl)-2H-benzo[b][1,4]oxazin-3(4H)-one (15 mg, 0.05 mmol) in THF (2.0 mL) were added trifluoroacetic anhydride (10.8 μL, 0.08 mmol) and pyridine (8.27 μL, 0.10 mmol) at room temperature and the mixture was stirred for 1 hr. The reaction mixture was diluted with EtOAc, washed with 1N HCl, brine, saturated aqueous NaHCO₃ and brine, dried (Na₂SO₄), concentrated and purified by preparative TLC (10% EtOAc in DCM) to give the title compound as a white solid (10 mg, 50%).

¹H-NMR (400 MHz, CDCl₃) δ: 8.06 (bs, 1H), 7.29-7.37 (m, 3H), 7.24 (d, J=2.0 Hz, 1H), 7.18 (dd, J=8.6, 2.0 Hz, 1H), 7.15 (m, 2H), 6.86 (d, J=8.6 Hz, 1H), 4.71 (dd, J=11.4, 5.1 Hz, 1H), 4.61 (s, 2H), 4.48 (t, 1H), 4.07 (dd, J=12.4, 5.1 Hz, 1H).

### Example 58

### 6-(1-Acetyl-4-phenyl-4,5-dihydro-1H-pyrazol-3-yl)-2H-benzo[b][1,4]oxazin-3(4H)-one

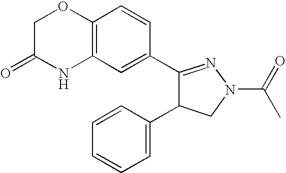

According to the method of Example 57, 6-(4-phenyl-4,5-dihydro-1H-pyrazol-3-yl)-2H-benzo[b][1,4]oxazin-3(4H)-one (50 mg, 0.17 mmol) in THF (2.0 mL) and acetic anhydride (24.2 μL, 0.26 mmol) were reacted to give the title compound as a white solid (30 mg, 52%).

¹H-NMR (400 MHz, CDCl₃) δ: 8.09 (bs, 1H), 7.25-7.33 (m, 3H), 7.14-7.19 (m, 4H), 6.85 (d, J=8.6 Hz, 1H), 4.64 (dd, J=11.7, 5.5 Hz, 1H), 4.60 (s, 2H), 4.37 (t, 1H), 4.01 (dd, J=12.3, 5.5 Hz, 1H), 2.46 (s, 3H); LCMS (ESI⁻), M−H⁻: 334.

### Example 59

### 6-(1-(Methylsulfonyl)-4-phenyl-4,5-dihydro-1H-pyrazol-3-yl)-2H-benzo[b][1,4]oxazin-3(4H)-one

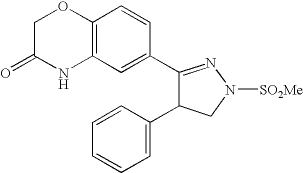

According to the method of Example 57, 6-(4-phenyl-4,5-dihydro-1H-pyrazol-3-yl)-2H-benzo[b][1,4]oxazin-3(4H)-one (50 mg, 0.17 mmol) and methanesulfonyl chloride (15.8 μL, 0.20 mmol) were reacted in DCM at 0° C. to give the title compound as a white solid (25 mg, 40%).

¹H-NMR (400 MHz, DMSO-d₆) δ: 10.74 (bs, 1H), 7.34 (m, 3H), 7.26 (m, 3H), 7.17 (dd, 1H), 6.91 (d, 1H), 4.95 (dd, 1H), 4.60 (s, 2H), 4.15 (t, 1H), 3.69 (dd, 1H), 3.11 (s, 3H);

LCMS (ESI⁻), M−H⁻: 370.

### Example 60

### 6-(1-Benzoyl-4-phenyl-4,5-dihydro-1H-pyrazol-3-yl)-2H-benzo[b][1,4]oxazin-3(4H)-one

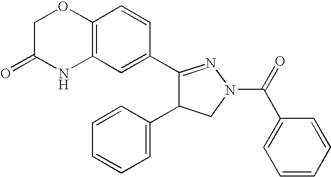

According to the method of Example 57, 6-(4-phenyl-4,5-dihydro-1H-pyrazol-3-yl)-2H-benzo[b][1,4]oxazin-3(4H)-one (35 mg, 0.12 mmol) and benzoyl chloride (20.8 μL, 0.18 mmol) were reacted to give the title compound as a white solid (15 mg, 31%).

¹H-NMR (400 MHz, CDCl₃) δ: 8.95 (bs, 1H), 8.04 (d, J=7.0 Hz, 2H), 7.45-7.51 (m, 3H), 7.31 (d, J=7.4 Hz, 2H), 7.25 (m, 1H), 7.19 (m, 3H), 7.15 (dd, J=8.6, 2.0 Hz, 1H), 6.82 (d, J=8.6 Hz, 1H), 4.64 (dd, J=11.5, 4.4 Hz, 1H), 4.56 (s, 2H), 4.56 (t, 1H), 4.20 (dd, J=11.7, 4.4 Hz, 1H); LCMS (APCI⁺), M+H⁺: 398.

### Example 61

### 6-(4-(4-Fluorophenyl)-5-oxo-2,5-dihydrofuran-3-yl)-2H-benzo[b][1,4]oxazin-3(4H)-one

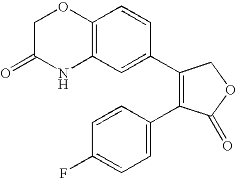

To a solution of 6-(2-chloroacetyl)-2H-benzo[b][1,4]oxazin-3(4H)-one (680 mg, 3.0 mmol) and 2-(4-fluorophenyl)acetic acid (422 mg, 2.74 mmol) in DMF (5.0 mL) was added dropwise triethylamine (0.420 mL, 3.01 mmol) at room temperature and the mixture was stirred overnight at room temperature. After cooling to 0° C., DBU (0.82 mL, 5.47 mmol) was added and the mixture was stirred for 1 hr at room temperature and heated to 40° C. for 1 hr. The dark reaction mixture was cooled, poured into ice-water, extracted twice with EtOAc, washed with 0.1N HCl solution, brine, saturated aqueous NaHCO₃ and brine, dried (MgSO₄) and concentrated in vacuo to give a yellow solid. The solid was slurried in DCM/EtOAc and sonicated, and ether was added. Vacuum filtration gave the title compound as a yellow solid (600 mg, 67%).

¹H-NMR (400 MHz, CDCl₃) δ: 8.80 (bs, 1H), 7.41 (dd, J=8.2, 5.4 Hz, 2H), 7.10 (t, J=8.6 Hz, 2H), 6.95 (s, 2H), 6.76 (s, 1H), 5.13 (s, 2H), 4.65 (s, 2H); LCMS (ESI⁻), M+H⁻: 324.

### Example 62

### 6-(5-Oxo-4-phenyl-2,5-dihydrofuran-3-yl)-2H-benzo[b][1,4]oxazin-3(4H)-one

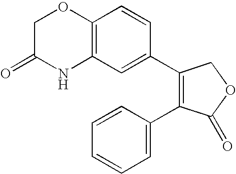

To a slurry of cesium carbonate (3.61 g, 11.1 mmol) in acetone was added 2-phenylacetic acid (905 mg, 6.64 mmol) and 6-(2-chloroacetyl)-2H-benzo[b][1,4]oxazin-3(4H)-one (500 mg, 2.21 mmol). The resulting mixture was heated at 65° C. for 12 hr. The mixture was cooled to room temperature and poured into water. The resulting solid was collected and purified by flash chromatography on silica gel (25%-50% EtOAc in hexane) to give the title compound as a green solid (191 mg, 28%).

¹H-NMR (400 MHz, DMSO-d₆) δ: 10.6 (s, 1H), 7.43 (d, 3H), 7.34 (d, J=7.8 Hz, 2H), 6.96 (s, 2H), 6.92 (s, 1H), 5.30 (s, 2H), 4.61 (s, 2H); LCMS (ESI⁺), M+H⁺: 308.

### Example 63

### 6-(2-Oxo-4-phenyl-2,5-dihydrofuran-3-yl)-2H-benzo[b][1,4]oxazin-3(4H)-one

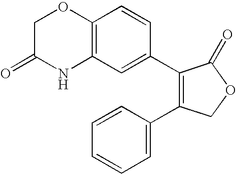

According to the method of Example 62, 2-(3-oxo-3,4-dihydro-2H-benzo[b][1,4]oxazin-6-yl)acetic acid (1.00 g, 4.83 mmol) and 2-chloro-1-phenylethanone (710 mg, 4.60 mmol) were reacted to give the title compound as an olive green solid (533 mg, 38%).

¹H-NMR (400 MHz, DMSO-d₆) δ: 10.8 (s, 1H), 7.44 (m, 5H), 6.98 (dd, J=5.4 Hz, 3.3 Hz, 2H), 6.85 (dd, J=8.3 Hz, 2.0 Hz, 1H), 5.36 (s, 2H), 4.62 (s, 2H); LCMS (ESI⁺), M+H⁺: 308.

### Example 64

### 6-(4-(4-Fluorophenyl)-2-oxo-2,5-dihydrofuran-3-yl)-2H-benzo[b][1,4]oxazin-3(4H)-one

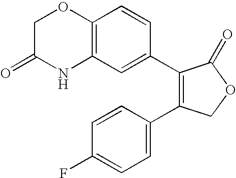

According to the method of Example 62, 2-(3-oxo-3,4-dihydro-2H-benzo[b][1,4]oxazin-6-yl)acetic acid (1.00 g, 4.83 mmol) and 2-chloro-1-(4-fluorophenyl)ethanone (793 mg, 4.60 mmol) were reacted to give the title compound as a green solid (469 mg, 31%).

¹H-NMR (400 MHz, DMSO-d₆) δ: 10.76 (s, 1H), 7.49 (dd, J=8.9 Hz, 5.5 Hz, 2H), 7.29 (t, J=8.9 Hz, 2H), 6.97 (d, J=8.3 Hz, 1H), 6.94 (d, J=2.0 Hz, 1H), 6.87 (dd, J=8.3 Hz, 2.0 Hz, 1H), 5.34 (s, 2H), 4.62 (s, 2H); LCMS (ESI⁺), M+H⁺: 326.

### Example 65

### 3-(4-Fluorophenyl)-4-(3-oxo-3,4-dihydro-2H-benzo[b][1,4]oxazin-6-yl)-1-phenyl-1H-pyrrole-2,5-dione

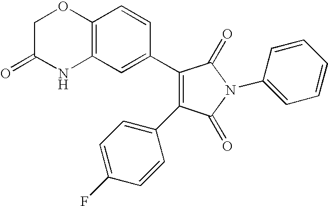

### 6-(4-(4-Fluorophenyl)-5-oxo-2,5-dihydrofuran-3-yl)-2H-benzo[b][1,4]oxazin-3(4H)-one

To a mixture of 2-(4-fluorophenyl)acetic acid (10.9 g, 70.9 mmol) and 6-(2-chloroacetyl)-2H-benzo[b][1,4]oxazin-3(4H)-one (16.0 g, 70.9 mmol) in acetone (750 mL) was added cesium carbonate (69.3 g, 213 mmol). The mixture was heated at 80° C. for 24 hr. After cooling to room temperature, water was added, and the mixture was extracted three times with EtOAc, dried (MgSO₄) and concentrated in vacuo to give the title compound as a yellow solid (21.1 g, 92%).

LCMS (ESI⁻), M−H⁻: 324.

### 3-(4-Fluorophenyl)-4-(3-oxo-3,4-dihydro-2H-benzo[b][1,4]oxazin-6-yl)-1-phenyl-1H-pyrrole-2,5-dione

To a solution of 6-(4-(4-fluorophenyl)-5-oxo-2,5-dihydrofuran-3-yl)-2H-benzo[b][1,4]oxazin-3(4H)-one (220 mg, 0.676 mmol) and aniline (63.0 mg, 0.676 mmol) in DMF (3.50 mL) was added p-toluenesulfonic acid monohydrate (6.4 mg, 0.034 mmol) and the resulting solution was heated at 150° C. for 12 hr. The reaction mixture was cooled and to the solution was added water (10 mL) to precipitate a brown solid. The filter cake was washed with ether to produce a yellow filtrate. The filtrate was evaporated to give the desired product as a yellow solid (95 mg, 34%).

¹H-NMR (400 MHz, DMSO-d₆) δ: 10.8 (s, 1H), 7.52 (m, 4H), 7.45 (m, 3H), 7.31 (t, J=8.7 Hz, 2H), 7.11 (s, 1H), 6.99 (s, 2H), 4.64 (s, 2H); LCMS (ESI⁻), M−H⁻: 413.

### Example 66

### 3-(4-Fluorophenyl)-4-(3-oxo-3,4-dihydro-2H-benzo[b][1,4]oxazin-6-yl)-1-(pyridin-3-yl)-1H-pyrrole-2,5-dione

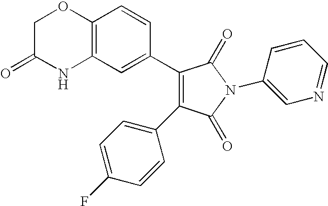

### 3-(4-Fluorophenyl)-4-(3-oxo-3,4-dihydro-2H-benzo[b][1,4]oxazin-6-yl)furan-2,5-dione

A solution of 6-(4-(4-fluorophenyl)-5-oxo-2,5-dihydrofuran-3-yl)-2H-benzo[b][1,4]oxazin-3(4H)-one (210 mg, 0.646 mmol) and DBU (295 mg, 1.94 mmol) was heated to 40° C. Through this solution was bubbled oxygen gas for 1 hr. The solution was heated for an additional 4 hr and then cooled to room temperature. The reaction mixture was then diluted with EtOAc and washed with 6N HCl and brine. The organic layer was dried (MgSO₄) and concentrated in vacuo to afford the title compound as a white solid (200 mg, 91%).

LCMS (ESI⁻), M−H⁻: 338.

### 3-(4-Fluorophenyl)-4-(3-oxo-3,4-dihydro-2H-benzo[b][1,4]oxazin-6-yl)-1-(pyridin-3-yl)-1H-pyrrole-2,5-dione

A solution of 3-(4-fluorophenyl)-4-(3-oxo-3,4-dihydro-2H-benzo[b][1,4]oxazin-6-yl)furan-2,5-dione (219 mg, 0.646 mmol) and pyridin-3-amine (122 mg, 1.29 mmol) in DMF was heated at 100° C. for 24 hr. Upon cooling, the solution was diluted with EtOAc and washed with water and brine. The organic layer was dried (MgSO₄) and concentrated in vacuo to afford the title compound as a yellow solid (199 mg, 74%).

¹H-NMR (400 MHz, DMSO-d₆) δ: 10.9 (s, 1H), 8.74 (d, J=2.3 Hz, 1H), 8.66 (dd, J=4.8, 1.6 Hz, 1H), 8.01 (m, 2H), 7.68 (m, 1H), 7.55 (dd, J=9.0, 5.6 Hz, 2H), 7.35 (t, J=9.0 Hz, 2H), 7.12 (s, 1H), 7.01 (s, 1H), 4.65 (s, 2H); LCMS (ESI⁺), M+H⁺: 416.

### Example 67

### 3-(4-Fluorophenyl)-4-(3-oxo-3,4-dihydro-2H-benzo[b][1,4]oxazin-6-yl)-1-(pyridin-2-yl)-1H-pyrrole-2,5-dione

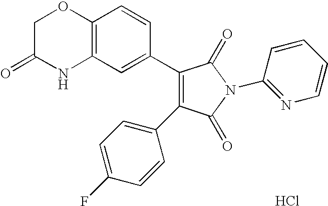

According to the method of Example 66, 3-(4-fluorophenyl)-4-(3-oxo-3,4-dihydro-2H-benzo[b][1,4]oxazin-6-yl)furan-2,5-dione (500 mg, 1.47 mmol) and pyridin-2-amine (277 mg, 2.94 mmol) gave the title compound as an orange solid (170 mg, 28%).

¹H-NMR (400 MHz, DMSO-d₆) δ: 10.90 (bs, 1H), 8.06 (m, 2H), 7.95 (m, 2H), 7.55 (m, 2H), 7.34 (t, J=8.8 Hz, 1H), 7.10 (s, 1H), 6.99 (m, 2H), 6.86 (t, J=6.4 Hz, 1H), 4.64 (s, 2H); LCMS (ESI⁺), M+H⁺: 416.

### Example 68

### 3-(4-Fluorophenyl)-4-(3-oxo-3,4-dihydro-2H-benzo[b][1,4]oxazin-6-yl)-1-(pyridin-4-yl)-1H-pyrrole-2,5-dione hydrochloride

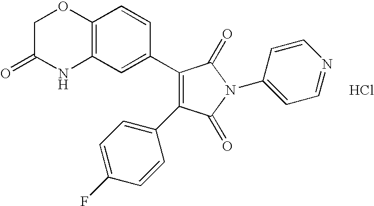

According to the method of Example 66, 3-(4-fluorophenyl)-4-(3-oxo-3,4-dihydro-2H-benzo[b][1,4]oxazin-6-yl)furan-2,5-dione (500 mg, 1.47 mmol) and pyridin-4-amine (277 mg, 2.94 mmol) gave the title compound as an orange solid (93 mg, 15%).

¹H-NMR (400 MHz, DMSO-d₆) δ: 10.90 (bs, 1H), 8.85 (d, J=6.5 Hz, 2H), 7.95 (d, J=6.0 Hz, 2H), 7.53 (dd, J=8.9 Hz, 5.6 Hz, 2H), 7.36 (t, J=8.8 Hz, 2H), 7.17 (m, 1H), 7.10 (s, 1H), 7.01 (s, 2H), 4.72 (s, 2H); LCMS (ESI⁺), M+H⁺: 416.

### Example 69

### 6-(4-(4-Fluorophenyl)-5-oxo-1-phenyl-2,5-dihydro-1H-pyrrol-3-yl)-2H-benzo[b][1,4]oxazin-3(4H)-one

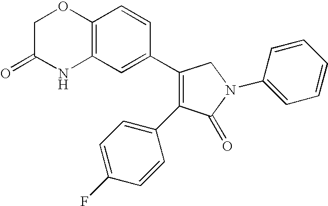

To a solution of 6-(4-(4-fluorophenyl)-5-oxo-2,5-dihydrofuran-3-yl)-2H-benzo[b][1,4]oxazin-3(4H)-one (250 mg, 0.769 mmol) in ethylene glycol (1.50 mL) in a microwave vessel were added aniline (215 mg, 2.31 mmol) and magnesium triflate (281 mg, 2.79 mmol) and the solution was degassed by bubbling N₂ through it for 5 min. The vessel was sealed and irradiated (250 W, 10 min, 140° C., 2 cycles). The crude reaction mixture was diluted with brine, extracted three times with EtOAc. The extract was dried (MgSO₄) and concentrated in vacuo. Purification of the residue by RP-HPLC (25%-100% acetonitrile/water, Biotage Horizon C18 column) gave the title compound as a yellow solid (21 mg, 7%).

¹H-NMR (400 MHz, DMSO-d₆) δ: 10.80 (s, 1H), 7.88 (s, 1H), 7.54 (m, 3H), 7.42 (m, 2H), 7.35 (m, 3H), 7.19 (m, 3H), 4.97 (s, 2H), 4.61 (s, 2H); LCMS (ESI⁻), M−H⁻: 399.

### Example 70

### 6-(1,4-Bis(4-fluorophenyl)-5-oxo-2,5-dihydro-1H-pyrrol-3-yl)-2H-benzo[b][1,4]oxazin-3(4H)-one

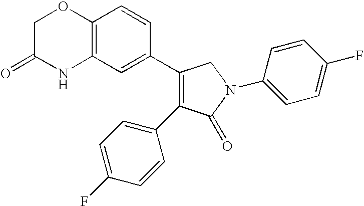

According to the method of Example 69, 6-(4-(4-fluorophenyl)-5-oxo-2,5-dihydrofuran-3-yl)-2H-benzo[b][1,4]oxazin-3(4H)-one (453 mg, 1.39 mmol) and 4-fluoroaniline (310 mg, 2.79 mmol) were reacted to give the title compound as a yellow solid (35 mg, 6%).

¹H-NMR (400 MHz, DMSO-d₆) δ: 10.8 (s, 1H), 7.91 (dd, J=9.1, 4.9 Hz, 2H), 7.38 (dd, J=8.9, 5.8 Hz, 2H), 7.29 (m, 4H), 7.07 (dd, J=8.4, 2.0 Hz, 1H), 6.99 (d, J=8.4 Hz, 1H), 6.88 (d, J=2.0 Hz, 1H), 4.97 (s, 2H), 4.60 (s, 2H); LCMS (ESI⁺), M+H⁺: 417.

### Example 71

### 6-(1-(4-Fluorophenyl)-3-(trifluoromethyl)-1H-pyrazol-5-yl)-2H-benzo[b][1,4]oxazin-3(4H)-one

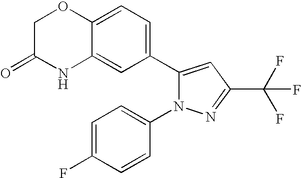

### 4,4,4-Trifluoro-1-(3-oxo-3,4-dihydro-2H-benzo[b][1,4]oxazin-6-yl)butane-1,3-dione

To a mixture of NaH (2.51 g, 105 mmol) in THF (100 mL) was carefully added ethyl 2,2,2-trifluoroacetate (12.5 mL, 105 mmol), observing both effervescence and a slight exotherm. To this resulting mixture were added sequentially 6-acetyl-2H-benzo[b][1,4]oxazin-3(4H)-one (5.00 g, 26.2 mmol), ethanol (2.50 mL) and a solution of [2,4]-dibenzo-18-crown-6 (150 mg, 0.418 mmol) in THF (50.0 mL). The mixture was refluxed for 16 hr, cooled, and partitioned between 10% H₂SO₄ (200 mL) and EtOAc (200 mL). The organic layer was separated and washed with water (200 mL), saturated aqueous NaHCO₃ (200 mL), water (200 mL) and brine (200 mL), dried (Na₂SO₄) and concentrated in vacuo. The residue was triturated with ether to give the title compound as a yellow solid (6.67 g, 80%).

¹H-NMR (400 MHz, DMSO-d₆) δ: 10.88 (s, 1H), 7.63 (dd, J=8.5, 2.1 Hz, 1H), 7.49 (d, J=2.1 Hz, 1H), 7.04 (d, J=8.4 Hz, 1H), 6.30 (s, 1H), 4.69 (s, 2H) and 10.81 (s, 1H), 7.58 (dd, J=8.4, 1.6 Hz, 1H), 7.48 (d, J=1.9 Hz, 1H), 7.11 (s, 1H), 7.00 (d, J=8.4 Hz, 1H), 4.67 (s, 2H), consistent with a mixture of enolic tautomers; LCMS (ESI⁻), M−H⁻: 286.

### 6-(1-(4-Fluorophenyl)-3-(trifluoromethyl)-1H-pyrazol-5-yl)-2H-benzo[b][1,4]oxazin-3(4H)-one

A solution of (4-fluorophenyl)hydrazine hydrochloride (133 mg, 0.823 mmol) and triethylamine (113 μL, 0.807 mmol) in isopropanol (4.60 mL) was stirred at room temperature for 15 min. To the reaction mixture was added 2,2,2-trifluoroacetic acid (129 μL, 1.68 mmol) and again stirred at room temperature for 15 min. To the resulting mixture was added 6-(4,4,4-trifluoro-3-hydroxybut-2-enoyl)-2H-benzo[b][1,4]oxazin-3(4H)-one (225 mg, 0.738 mmol) and the reaction mixture was heated at 60° C. overnight. The reaction mixture was concentrated in vacuo to remove most of the isopropanol, water (20.0 mL) was added, and the pH adjusted to 5-6 with 1M NaOH. The resulting solids were collected and washed with petroleum ether to give the title compound as a tan solid (198 mg, 67%).

¹H-NMR (400 MHz, CDCl₃) δ: 8.28 (s, 1H), 7.31 (dd, J=9.0, 4.7 Hz, 2H), 7.10 (dd, J=9.0, 8.2 Hz, 2H), 6.93 (d, J=8.4 Hz, 1H), 6.80 (dd, J=8.4, 2.0 Hz, 1H), 6.71 (s, 1H), 6.65 (d, J=2.0 Hz, 1H), 4.65 (s, 2H); LCMS (ESI⁺), M+H⁺: 378.

### Example 72

### 6-(1-(3-Fluorophenyl)-3-(trifluoromethyl)-1H-pyrazol-5-yl)-2H-benzo[b][1,4]oxazin-3(4H)-one

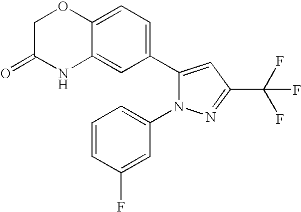

According to the method of Example 71, 6-(4,4,4-trifluoro-3-hydroxybut-2-enoyl)-2H-benzo[b][1,4]oxazin-3(4H)-one (225 mg, 0.738 mmol) and (3-fluorophenyl)hydrazine hydrochloride (134 mg, 0.823 mmol) were reacted to give the title compound as a tan solid (256 mg, 86%).

¹H-NMR (400 MHz, CDCl₃) δ: 7.81 (s, 1H), 7.35 (m, 1H), 7.10 (m, 3H), 6.96 (d, J=8.4 Hz, 1H), 6.84 (dd, J=8.4, 1.9 Hz, 1H), 6.71 (s, 1H), 6.66 (d, J=1.9 Hz, 1H), 4.67 (s, 2H); LCMS (ESI⁺), M+H⁺: 378.

### Example 73

### 6-(1-(2-Fluorophenyl)-3-(trifluoromethyl)-1H-pyrazol-5-yl)-2H-benzo[b][1,4]oxazin-3(4H)-one

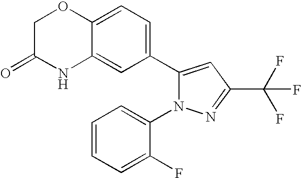

According to the method of Example 71, 6-(4,4,4-trifluoro-3-hydroxybut-2-enoyl)-2H-benzo[b][1,4]oxazin-3(4H)-one (225 mg, 0.738 mmol) and (2-fluorophenyl)hydrazine hydrochloride (134 mg, 0.823 mmol) were reacted to give the title compound as a tan solid (255 mg, 85%).

¹H-NMR (400 MHz, CDCl₃) δ: 7.62 (s, 1H), 7.53 (m, 1H), 7.45 (m, 1H), 7.28 (bt, J=7.6 Hz, 1H), 7.12 (m, 1H), 6.90 (d, J=8.4 Hz, 1H), 6.80 (dd, J=8.4, 2.0 Hz, 1H), 6.73 (s, 1H), 6.65 (d, J=2.0 Hz, 1H), 4.63 (s, 2H); LCMS (ESI⁺), M+H⁺: 378.

### Example 74

### 6-(1-(4-Chlorophenyl)-3-(trifluoromethyl)-1H-pyrazol-5-yl)-2H-benzo[b][1,4]oxazin-3(4H)-one

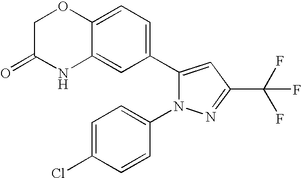

According to the method of Example 71, 6-(4,4,4-trifluoro-3-hydroxybut-2-enoyl)-2H-benzo[b][1,4]oxazin-3(4H)-one (225 mg, 0.738 mmol) and (4-chlorophenyl)hydrazine hydrochloride (147 mg, 0.823 mmol) were reacted to give the title compound as a tan solid (209 mg, 66%).

¹H-NMR (400 MHz, CDCl₃) δ: 7.86 (s, 1H), 7.37 (d, J=8.6 Hz, 2H), 7.27 (d, J=8.6 Hz, 2H), 6.95 (d, J=8.4 Hz, 1H), 6.82 (dd, J=8.4, 2.0 Hz, 1H), 6.70 (s, 1H), 6.65 (d, J=2.0 Hz, 1H), 4.66 (s, 2H); LCMS (ESI⁺), M+H⁺: 394.

### Example 75

### 6-(1-p-Tolyl-3-(trifluoromethyl)-1H-pyrazol-5-yl)-2H-benzo[b][1,4]oxazin-3(4H)-one

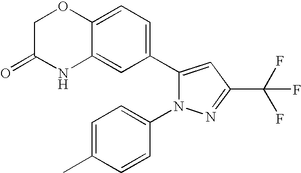

According to the method of Example 71, 6-(4,4,4-trifluoro-3-hydroxybut-2-enoyl)-2H-benzo[b][1,4]oxazin-3(4H)-one (225 mg, 0.738 mmol) and p-tolylhydrazine hydrochloride (131 mg, 0.823 mmol) were reacted to give the title compound as a light beige solid (246 mg, 83%).

¹H-NMR (400 MHz, CDCl₃) δ: 7.73 (s, 1H), 7.26 (s, 2H), 6.93 (d, J=8.4 Hz, 1H), 6.84 (dd, J=8.4, 2.0 Hz, 1H), 6.69 (s, 1H), 6.61 (d, J=2.0 Hz, 1H), 4.64 (s, 2H), 2.39 (s, 3H); LCMS (ESI⁺), M+H⁺: 374.

### Example 76

### 6-(1-(4-Methoxyphenyl)-3-(trifluoromethyl)-1H-pyrazol-5-yl)-2H-benzo[b][1,4]oxazin-3(4H)-one

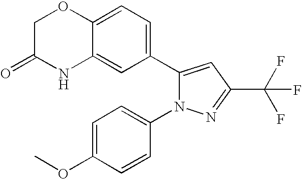

According to the method of Example 71, 6-(4,4,4-trifluoro-3-hydroxybut-2-enoyl)-2H-benzo[b][1,4]oxazin-3(4H)-one (225 mg, 0.738 mmol) and (4-methoxyphenyl)hydrazine hydrochloride (144 mg, 0.823 mmol) were reacted to give the title compound as a dark beige solid (225 mg, 74%).

¹H-NMR (400 MHz, CDCl₃) δ: 7.72 (s, 1H), 7.24 (d, J=9.0 Hz, 2H) 6.93 (d, J=8.4 Hz, 1H), 6.89 (d, J=9.3, 2.0 Hz, 2H), 6.84 (dd, J=8.4, 2.0 Hz, 1H), 6.69 (s, 1H), 6.61 (d, J=2.0 Hz, 1H), 4.64 (s, 2H), 3.84 (s, 3H); LCMS (ESI⁺), M+H⁺: 390.

### Example 77

### 6-(1-(3,4-Dichlorophenyl)-3-(trifluoromethyl)-1H-pyrazol-5-yl)-2H-benzo[b][1,4]oxazin-3(4H)-one

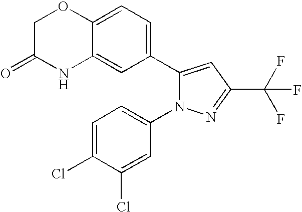

According to the method of Example 71, 6-(4,4,4-trifluoro-3-hydroxybut-2-enoyl)-2H-benzo[b][1,4]oxazin-3(4H)-one (200 mg, 0.696 mmol) and (3,4-dichlorophenyl)hydrazine hydrochloride (156 mg, 0.731 mmol) were reacted to give the title compound as a light beige solid (238 mg, 78%).

¹H-NMR (400 MHz, CDCl₃) δ: 7.92 (s, 1H), 7.57 (d, J=2.5 Hz, 1H), 7.43 (d, J=8.6 Hz, 1H), 7.08 (dd, J=8.6, 2.5 Hz, 1H), 6.98 (d, J=8.3 Hz, 1H), 6.83 (dd, J=8.3, 2.0 Hz, 1H), 6.71 (s, 1H), 6.67 (d, J=2.0 Hz, 1H), 4.68 (s, 2H);

LCMS (ESI⁺), M+H⁺: 429.

### Example 78

### 6-(1-(2,4-Difluorophenyl)-3-(trifluoromethyl)-1H-pyrazol-5-yl)-2H-benzo[b][1,4]oxazin-3(4H)-one

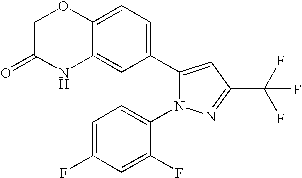

According to the method of Example 71, 6-(4,4,4-trifluoro-3-hydroxybut-2-enoyl)-2H-benzo[b][1,4]oxazin-3(4H)-one (225 mg, 0.738 mmol) and (2,4-difluorophenyl)hydrazine hydrochloride (149 mg, 0.823 mmol) were reacted to give the title compound as a light beige solid (267 mg, 84%).

¹H-NMR (400 MHz, CDCl₃) δ: 7.83 (s, 1H), 7.52 (m, 1H), 7.02 (m, 1H), 6.91 (d, J=8.3 Hz, 1H), 6.88 (m, 1H), 6.78 (dd, J=8.4, 2.0 Hz, 1H), 6.73 (s, 1H), 6.67 (d, J=2.0 Hz, 1H), 4.64 (s, 2H); LCMS (ESI⁺), M+H⁺: 396.

### Example 79

### 6-(3-(Trifluoromethyl)-1-(4-trifluoromethylphenyl)-1H-pyrazol-5-yl)-2H-benzo[b][1,4]oxazin-3(4H)-one

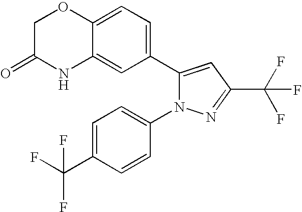

According to the method of Example 71 and in the absence of triethylamine, 6-(4,4,4-trifluoro-3-hydroxybut-2-enoyl)-2H-benzo[b][1,4]oxazin-3(4H)-one (200 mg, 0.696 mmol) and (4-trifluoromethylphenyl)hydrazine (129 mg, 0.731 mmol) were reacted to give the title compound as a light beige solid (252 mg, 84%).

¹H-NMR (400 MHz, CDCl₃) δ: 8.07 (s, 1H), 7.66 (d, J=8.5 Hz, 2H), 7.47 (d, J=8.5 Hz, 2H), 6.97 (d, J=8.4 Hz, 1H), 6.81 (dd, J=8.4, 2.0 Hz, 1H), 6.73 (s, 1H), 6.69 (d, J=2.0 Hz, 1H), 4.67 (s, 2H); LCMS (ESI⁺), M+H⁺: 428.

### Example 80

### 6-(1-(Pyridin-2-yl)-3-(trifluoromethyl)-1H-pyrazol-5-yl)-2H-benzo[b][1,4]oxazin-3(4H)-one

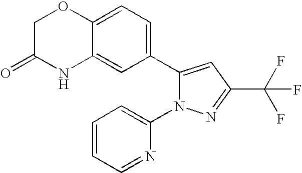

According to the method of Example 71 and in the absence of triethylamine, 6-(4,4,4-trifluoro-3-hydroxybut-2-enoyl)-2H-benzo[b][1,4]oxazin-3(4H)-one (225 mg, 0.738 mmol) and (pyridin-2-yl)hydrazine (90.0 mg, 0.823 mmol) were reacted to give, after flash chromatography on silica gel (10-30% EtOAc in petroleum ether), the title compound as a pale yellow solid (159 mg, 54%).

¹H-NMR (400 MHz, CDCl₃) δ: 8.39 (ddd, J=4.8, 2.0, 1.0 Hz, 1H), 7.82 (ddd, J=7.8, 7.8, 2.0 Hz, 1H), 7.75 (bs, 1H), 7.63 (ddd, J=7.8, 1.0, 1.0 Hz, 1H), 7.33 (ddd, J=7.4, 4.8, 1.0 Hz, 1H), 6.92 (d, J=8.4 Hz, 1H), 6.83 (dd, J=8.4, 2.0 Hz, 1H), 6.75 (d, J=2.0 Hz, 1H), 6.71 (s, 1H), 4.65 (s, 2H); LCMS (ESI⁺), M+H⁺: 361.

### Example 81

### 6-(1-(2-Chlorophenyl)-3-(trifluoromethyl)-1H-pyrazol-5-yl)-2H-benzo[b][1,4]oxazin-3(4H)-one

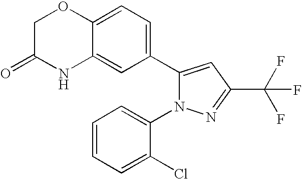

According to the method of Example 71, 6-(4,4,4-trifluoro-3-hydroxybut-2-enoyl)-2H-benzo[b][1,4]oxazin-3(4H)-one (225 mg, 0.738 mmol) and (2-chlorophenyl)hydrazine hydrochloride (147 mg, 0.823 mmol) were reacted to give the title compound as a light beige solid (241 mg, 76%).

¹H-NMR (400 MHz, CDCl₃) δ: 7.66 (s, 1H), 7.47 (dd, J=7.4, 2.0 Hz, 1H), 7.41-7.47 (m, 2H), 7.40 (dd, J=7.4, 2.0 Hz, 1H), 6.88 (d, J=8.4 Hz, 1H), 6.79 (dd, J=8.4, 2.0 Hz, 1H), 6.74 (s, 1H), 6.97 (d, J=2.0 Hz, 1H), 4.62 (s, 2H);

LCMS (ESI⁺), M+H⁺: 394.

### Example 82

### 6-(1-o-Tolyl-3-(trifluoromethyl)-1H-pyrazol-5-yl)-2H-benzo[b][1,4]oxazin-3(4H)-one

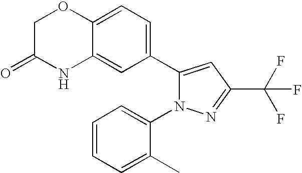

A mixture of 1-o-tolylhydrazine hydrochloride (131 mg, 0.82 mmol) and triethylamine (112 μL, 0.807 mmol) in IPA (4.6 mL), was stirred at room temperature for 15 min. To the mixture was added TFA (129 μL, 1.68 mmol) and stirring was continued for 15 minutes. 4,4,4-Trifluoro-1-(3-oxo-3,4-dihydro-2H-benzo[b][1,4]oxazin-6-yl)butane-1,3-dione (225 mg, 0.78 mmol) was added and the reaction mixture was heated to 60° C. overnight. Most of the IPA was removed in vacuo, water (20 mL) was added, and the pH adjusted to 5-6 with 1M NaOH. The resultant solids were collected by filtration, washed with petroleum ether and dried, giving the title compound as a beige solid (216 mg, 73%).

¹H-NMR (400 MHz, CDCl₃) δ: 7.69 (s, 1H), 7.37 (m, 1H), 7.27 (m, 3H), 6.87 (d, J=8.4 Hz, 1H), 6.79 (dd, J=8.4, 2.0 Hz, 1H), 6.75 (s, 1H), 6.53 (d, J=2.0 Hz, 1H), 4.61 (s, 2H), 1.97 (s, 3H); LCMS (ESI⁺), M+H⁺: 374.

### Example 83

### 6-(3-(Trifluoromethyl)-1-(2-trifluoromethylphenyl)-1H-pyrazol-5-yl)-2H-benzo[b][1,4]oxazin-3(4H)-one

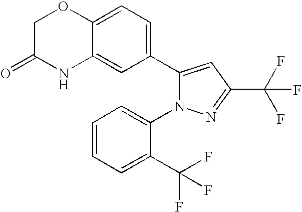

According to the method of Example 71 and in the absence of triethylamine, 6-(4,4,4-trifluoro-3-hydroxybut-2-enoyl)-2H-benzo[b][1,4]oxazin-3(4H)-one (200 mg, 0.696 mmol) and (2-(trifluoromethyl)phenyl)hydrazine (129 mg, 0.731 mmol) were reacted to give the title compound as orange crystals (155 mg, 49%) after recrystallization from isopropanol/water.

¹H-NMR (400 MHz, CDCl₃) δ: 8.07 (s, 1H), 7.81 (m, 1H), 7.63 (m, 2H), 7.38 (m, 1H), 6.86 (d, J=8.4 Hz, 1H), 6.75 (s, 1H), 6.74 (dd, J=8.4, 2.0 Hz, 1H), 6.61 (d, J=2.0 Hz, 1H), 4.61 (s, 2H); LCMS (ESI⁺), M+H⁺: 428.

### Example 84

### 6-(1-m-Tolyl-3-(trifluoromethyl)-1H-pyrazol-5-yl)-2H-benzo[b][1,4]oxazin-3(4H)-one

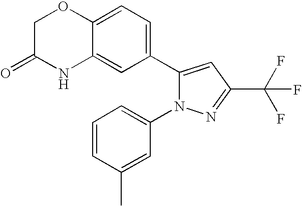

According to the method of Example 71 and in the absence of triethylamine, 6-(4,4,4-trifluoro-3-hydroxybut-2-enoyl)-2H-benzo[b][1,4]oxazin-3(4H)-one (225 mg, 0.738 mmol) and m-tolylhydrazine (101 mg, 0.823 mmol) were reacted to give the title compound as an orange/red solid (31 mg, 10%) after recrystallization from ethanol/water.

¹H-NMR (400 MHz, CDCl₃) δ: 7.93 (s, 1H), 7.18-7.26 (m, 3H), 6.98 (m, 1H), 6.92 (d, J=8.3 Hz, 1H), 6.83 (dd, J=8.3, 2.0 Hz, 1H), 6.70 (s, 1H), 6.64 (d, J=8.3 Hz, 1H), 4.64 (s, 2H), 2.37 (s, 3H); LCMS (ESI⁺), M+H⁺: 374.

### Example 85

### 6-(1-(2-Methoxyphenyl)-3-(trifluoromethyl)-1H-pyrazol-5-yl)-2H-benzo[b][1,4]oxazin-3(4H)-one

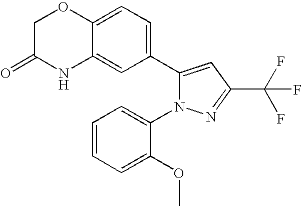

According to the method of Example 71, 6-(4,4,4-trifluoro-3-hydroxybut-2-enoyl)-2H-benzo[b][1,4]oxazin-3(4H)-one (225 mg, 0.738 mmol) and (2-methoxyphenyl)hydrazine hydrochloride (144 mg, 0.823 mmol) were reacted to give the title compound as a pinkish tan solid (250 mg, 77%).

¹H-NMR (400 MHz, CDCl₃) δ: 7.93 (s, 1H), 7.41 (m, 2H), 7.04 (ddd, J=7.8, 7.8, 1.2 Hz, 1H), 6.92 (d, J=8.2 Hz, 1H), 6.87 (d, J=8.4 Hz, 1H), 6.83 (dd, J=8.4, 1.8 Hz, 1H), 6.70 (s, 1H), 6.62 (d, J=1.8 Hz, 1H), 4.61 (s, 2H), 3.57 (s, 1H); LCMS (ESI⁺), M+H⁺: 390.

### Example 86

### 6-(1-(4-Chloro-2-methylphenyl)-3-(trifluoromethyl)-1H-pyrazol-5-yl)-2H-benzo[b][1,4]oxazin-3(4H)-one

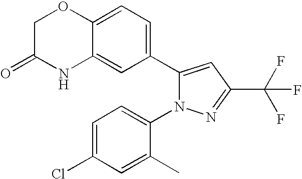

A mixture of 1-o-tolylhydrazine hydrochloride (131 mg, 0.82 mmol) and triethylamine (112 μL, 0.807 mmol) in IPA (4.6 mL), was stirred at room temperature for 15 min. To the mixture was added TFA (129 μL, 1.68 mmol) and stirring was continued for 15 minutes. 4,4,4-Trifluoro-1-(3-oxo-3,4-dihydro-2H-benzo[b][1,4]oxazin-6-yl)butane-1,3-dione (225 mg, 0.78 mmol) was added and the reaction mixture was heated to 60° C. overnight. Most of the IPA was removed in vacuo, water (20 mL) was added, and the pH adjusted to 5-6 with 1M NaOH. The resultant solids were collected by filtration, washed with petroleum ether and dried, giving the title compound as a beige solid (216 mg, 73%).

¹H-NMR (400 MHz, CDCl₃) δ: 8.40 (s, 1H), 7.27 (dd, J=8.2, 2.0 Hz, 1H), 7.25 (d, J=2.0 Hz, 1H), 7.20 (d, J=8.2 Hz, 1H), 6.89 (d, J=8.2 Hz, 1H), 6.76 (dd, J=8.2, 2.0 Hz, 1H), 6.75 (s, 1H), 6.60 (d, J=2.0 Hz, 1H), 4.64 (s, 2H);

LCMS (ESI⁺), M+H⁺: 408.

### Example 87

### 6-(1-(4-Ethylphenyl)-3-(trifluoromethyl)-1H-pyrazol-5-yl)-2H-benzo[b][1,4]oxazin-3(4H)-one

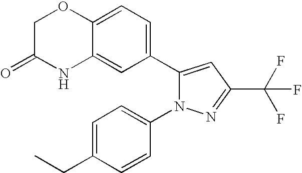

According to the method of Example 71, 6-(4,4,4-trifluoro-3-hydroxybut-2-enoyl)-2H-benzo[b][1,4]oxazin-3(4H)-one (225 mg, 0.738 mmol) and (4-ethylphenyl)hydrazine hydrochloride (142 mg, 0.823 mmol) were reacted to give the title compound as a beige solid (252 mg, 79%).

¹H-NMR (400 MHz, CDCl₃) δ: 7.83 (s, 1H), 7.23 (s, 4H), 6.92 (d, J=8.4 Hz, 1H), 6.84 (dd, J=8.4, 2.0 Hz, 1H), 6.69 (s, 1H), 6.66 (d, J=2.0 Hz, 1H), 4.64 (s, 2H), 2.68 (q, J=7.6 Hz, 2H), 1.24 (t, J=7.6 Hz, 3H); LCMS (ESI⁺), M+H⁺: 388.

### Example 88

### 6-(1-(4-Isopropylphenyl)-3-(trifluoromethyl)-1H-pyrazol-5-yl)-2H-benzo[b][1,4]oxazin-3(4H)-one

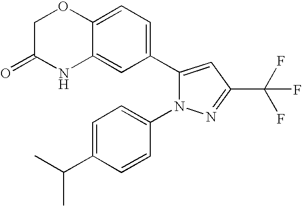

According to the method of Example 71, 6-(4,4,4-trifluoro-3-hydroxybut-2-enoyl)-2H-benzo[b][1,4]oxazin-3(4H)-one (225 mg, 0.738 mmol) and (4-isopropylphenyl)hydrazine hydrochloride (154 mg, 0.823 mmol) were reacted to give the title compound as a tan solid (270 mg, 80%).

¹H-NMR (400 MHz, CDCl₃) δ: 7.89 (s, 1H), 7.26 (s, 2H), 7.23 (s, 2H), 6.92 (d, J=8.4 Hz, 1H), 6.84 (dd, J=8.4, 2.0 Hz, 1H), 6.69 (s, 1H), 6.66 (d, J=2.0 Hz, 1H), 4.65 (s, 2H), 2.87 (sept, J=6.6 Hz, 1H), 1.25 (d, J=6.6 Hz, 6H);

LCMS (ESI⁺), M+H⁺: 402.

### Example 89

### 6-(1-(4-Trifluoromethoxyphenyl)-3-(trifluoromethyl)-1H-pyrazol-5-yl)-2H-benzo[b][1,4]oxazin-3(4H)-one

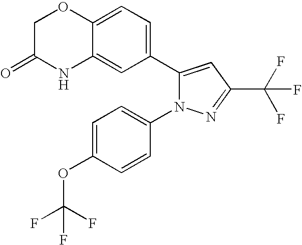

According to the method of Example 71, 6-(4,4,4-trifluoro-3-hydroxybut-2-enoyl)-2H-benzo[b][1,4]oxazin-3(4H)-one (200 mg; 0.696 mmol) and (4-(trifluoromethoxy)phenyl)hydrazine hydrochloride (167 mg, 0.731 mmol) were reacted to give the title compound as a beige solid (224 mg, 69%).

¹H-NMR (400 MHz, DMSO-d₆) δ: 8.29 (s, 1H), 7.37 (d, J=9.0 Hz, 2H), 7.24 (d, J=9.0 Hz, 2H), 6.95 (d, J=8.3 Hz, 1H), 6.80 (dd, J=8.3, 2.0 Hz, 1H), 6.72 (s, 1H), 6.70 (d, J=2.0 Hz, 1H), 4.66 (s, 2H); LCMS (ESI⁺), M+H⁺: 444.

### Example 90

### 6-(1-(4-Propylphenyl)-3-(trifluoromethyl)-1H-pyrazol-5-yl)-2H-benzo[b][1,4]oxazin-3(4H)-one

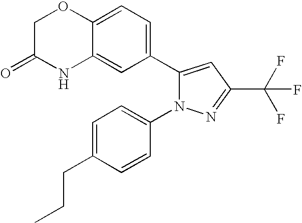

According to the method of Example 71, 6-(4,4,4-trifluoro-3-hydroxybut-2-enoyl)-2H-benzo[b][1,4]oxazin-3(4H)-one (225 mg, 0.738 mmol) and (4-propylphenyl)hydrazine hydrochloride (154 mg, 0.823 mmol) were reacted to give the title compound as a beige solid (257 mg, 78%).

¹H-NMR (400 MHz, CDCl₃) δ: 7.68 (s, 1H), 7.20 (m, 4H), 6.92 (d, J=8.4 Hz, 1H), 6.84 (dd, J=8.4, 2.0 Hz, 1H), 6.69 (s, 1H), 6.62 (d, J=2.0 Hz, 1H), 4.64 (s, 2H), 2.62 (t, J=7.6 Hz, 2H), 1.64 (m, 2H), 0.93 (t, J=7.2 Hz, 3H); LCMS (ESI⁺), M+H⁺: 402.

### Example 91

### 6-(1-Phenethyl-3-(trifluoromethyl)-1H-pyrazol-5-yl)-2H-benzo[b][1,4]oxazin-3(4H)-one

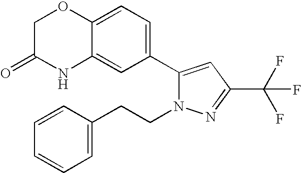

According to the method of Example 71, 6-(4,4,4-trifluoro-3-hydroxybut-2-enoyl)-2H-benzo[b][1,4]oxazin-3(4H)-one (225 mg, 0.738 mmol) and phenelzine sulfate salt (193 mg, 0.823 mmol) were reacted to give the title compound as an ivory solid (276 mg, 91%).

¹H-NMR (400 MHz, CD₂Cl₂) δ: 7.43 (s, 1H), 7.21 (m, 3H), 6.93 (d, J=8.3 Hz, 1H), 6.89 (m, 2H), 6.64 (dd, J=8.3, 2.0 Hz, 1H), 6.38 (s, 1H), 6.03 (d, J=2.0 Hz, 1H), 4.61 (s, 2H), 4.26 (t, J=6.8 Hz, 2H), 3.12 (t, J=6.8 Hz, 2H);

LCMS (ESI⁺), M+H⁺: 388.

### Example 92

### 6-(1-Benzyl-3-(trifluoromethyl)-1H-pyrazol-5-yl)-2H-benzo[b][1,4]oxazin-3(4H)-one

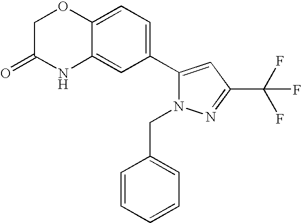

According to the method of Example 71, 6-(4,4,4-trifluoro-3-hydroxybut-2-enoyl)-2H-benzo[b][1,4]oxazin-3(4H)-one (225 mg, 0.738 mmol) and benzylhydrazine dihydrochloride (161 mg, 0.823 mmol) were reacted to give the title compound as a beige solid (234 mg, 76%).

¹H-NMR (400 MHz, CDCl₃) δ: 7.91 (s, 1H), 7.34-7.28 (m, 3H), 7.05 (m, 2H), 7.01 (d, J=8.3 Hz, 1H), 6.90 (dd, J=8.3, 2.0 Hz, 1H), 6.61 (d, J=2.0 Hz, 1H), 6.55 (s, 1H), 5.34 (s, 2H), 4.66 (s, 2H); LCMS (ESI⁺), M+H⁺: 374.

### Example 93

### 6-(1-(2,5-Dimethylphenyl)-3-(trifluoromethyl)-1H-pyrazol-5-yl)-2H-benzo[b][1,4]oxazin-3(4H)-one

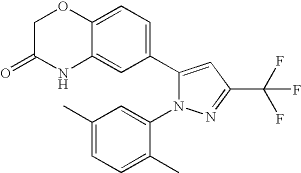

A mixture of 1-(2,5-dimethylphenyl)hydrazine hydrochloride (142 mg, 0.823 mmol) and triethylamine (113 μL, 0.807 mmol) in IPA (4.6 mL), was stirred at room temperature for 15 min. To the mixture was added TFA (129 μL, 1.68 mmol) and stirring was continued for 15 minutes. 4,4,4-Trifluoro-1-(3-oxo-3,4-dihydro-2H-benzo[b][1,4]oxazin-6-yl)butane-1,3-dione (225 mg, 0.78 mmol) was added and the reaction mixture was heated to 60° C. overnight. Most of the IPA was removed in vacuo, water (20 mL) was added, and the pH adjusted to 5-6 with 1M NaOH. The resultant solids were collected by filtration, washed with petroleum ether and dried, giving the title compound as a pale yellow solid (92 mg, 30%) after recrystallization from ethanol/water.

¹H-NMR (400 MHz, CDCl₃) δ: 10.79 (brs, 1H), 7.24 (m, 3H), 7.10 (s, 1H), 6.90 (d, J=9.0 Hz, 1H), 6.79 (m, 2H), 4.58 (s, 2H), 2.31 (s, 3H), 1.80 (s, 3H); LCMS (ESI⁺), M+H⁺: 388.

### Example 94

### 6-(1-(Naphthalen-1-yl)-3-(trifluoromethyl)-1H-pyrazol-5-yl)-2H-benzo[b][1,4]oxazin-3(4H)-one

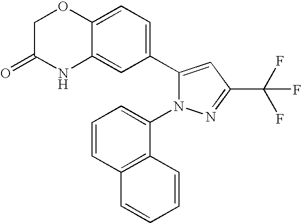

According to the method of Example 71, 6-(4,4,4-trifluoro-3-hydroxybut-2-enoyl)-2H-benzo[b][1,4]oxazin-3(4H)-one (225 mg, 0.738 mmol) and (naphthalen-1-yl)hydrazine hydrochloride (160 mg, 0.823 mmol) were reacted to give, after flash chromatography on silica gel (10-30% EtOAc in petroleum ether), the title compound as a reddish brown solid (55 mg, 17%).

¹H-NMR (400 MHz, CD₂Cl₂) δ: 7.99 (d, J=8.6 Hz, 1H), 7.94 (d, J=9.0 Hz, 1H), 7.56-7.46 (m, 4H), 7.40 (dd, J=7.3, 1.2 Hz, 1H), 7.36 (bd, J=8.2 Hz, 1H), 6.86 (s, 1H), 6.73 (m, 2H), 6.49 (d, J=1.2 Hz, 1H), 4.49 (s, 2H); LCMS (ESI⁺), M+H⁺: 410.

### Example 95

### 6-(1-(2-Ethylphenyl)-3-(trifluoromethyl)-1H-pyrazol-5-yl)-2H-benzo[b][1,4]oxazin-3(4H)-one

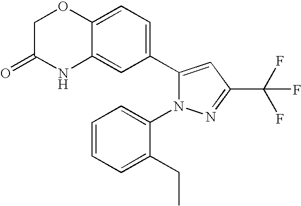

According to the method of Example 71, 6-(4,4,4-trifluoro-3-hydroxybut-2-enoyl)-2H-benzo[b][1,4]oxazin-3(4H)-one (225 mg, 0.738 mmol) and (2-ethylphenyl)hydrazine hydrochloride (142 mg, 0.823 mmol) were reacted to give, after flash chromatography on silica gel (10-30% EtOAc in petroleum ether), the title compound as a yellow solid (143 mg, 47%).

¹H-NMR (400 MHz, CD₂Cl₂) δ: 7.47 (bs, 1H), 7.42 (ddd, J=7.8, 7.0, 2.0 Hz, 1H), 7.34 (ddd, J=7.8, 2.0 Hz, 1H), 7.26 (ddd, J=7.8, 7.0, 1.6 Hz, 1H), 7.21 (dd, J=7.8, 1.6 Hz, 1H), 6.84 (d, J=8.4 Hz, 1H), 6.78 (dd, J=8.4, 2.1 Hz, 1H), 6.76 (s, 1H), 6.52 (d, J=2.1 Hz, 1H), 4.56 (s, 2H), 2.30 (q, J=7.6 Hz, 2H), 1.02 (t, J=7.6 Hz, 3H); LCMS (ESI⁺), M+H⁺: 388.

### Example 96

### 6-(1-Phenyl-3-(trifluoromethyl)-1H-pyrazol-5-yl)-2H-benzo[b][1,4]oxazin-3(4H)-one

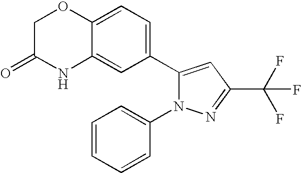

A stirred solution of 6-(4,4,4-trifluoro-3-hydroxybut-2-enoyl)-2H-benzo[b][1,4]oxazin-3(4H)-one (750 mg, 2.61 mmol) and phenylhydrazine (278 μL, 2.74 mmol) in ethanol was heated at 60° C. overnight and then concentrated in vacuo. To the residue was added ice-water and the resulting mixture was acidified with 6N HCl and extracted with EtOAc. The combined organic layer was washed with water and brine, dried (Na₂SO₄), and concentrated in vacuo. The residue was triturated with petroleum ether and purified by flash chromatography on silica gel (30% EtOAc in petroleum ether) to give the title compound as an off-white solid (171 mg, 18%).

¹H-NMR (400 MHz, CDCl₃) δ: 8.09 (s, 1H), 7.39 (m, 3H), 7.32 (m, 2H), 6.92 (d, J=8.2 Hz, 1H), 6.82 (dd, J=8.2, 2.0 Hz, 1H), 6.71 (s, 1H), 6.64 (d, J=2.0 1H), 4.64 (s, 2H);

LCMS (ESI⁺), M+H⁺: 360.

### Example 97

### 6-(3-Methyl-1-phenyl-1H-pyrazol-5-yl)-2H-benzo[b][1,4]oxazin-3(4H)-one

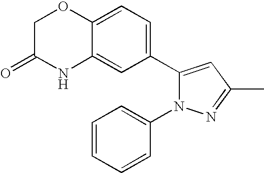

### 6-(3-Hydroxybut-2-enoyl)-2H-benzo[b][1,4]oxazin-3(4H)-one

According to the method of Example 71, 6-acetyl-2H-benzo[b][1,4]oxazin-3(4H)-one (1.0 g, 5.23 mmol) and EtOAc (2.04 mL, 20.9 mmol) were reacted to give the title compound as a tan solid (620 mg, 51%).

¹H-NMR (400 MHz, CDCl₃) δ: 8.26 (s, 1H), 7.51 (dd, J=8.2, 2.0 Hz, 1H), 7.40 (d, J=2.0 Hz, 1H), 7.01 (d, J=8.2 Hz, 1H), 6.10 (s, 1H), 4.70 (s, 2H), 2.19 (s, 3H).

### 6-(3-Methyl-1-phenyl-1H-pyrazol-5-yl)-2H-benzo[b][1,4]oxazin-3(4H)-one

According to the method of Example 96, 6-(3-hydroxybut-2-enoyl)-2H-benzo[b][1,4]oxazin-3(4H)-one (620 mg, 2.66 mmol) and phenylhydrazine (283 μL, 2.79 mmol) were reacted to give the title compound as a yellow solid (170 mg, 21%).

¹H-NMR (400 MHz, CDCl₃) δ: 7.84 (s, 1H), 7.31 (m, 5H), 6.89 (d, J=8.2 Hz, 1H), 6.82 (dd, J=8.2, 1.9 Hz, 1H), 6.62 (d, J=1.9 Hz, 1H), 6.26 (s, 1H), 4.62 (s, 2H), 2.37 (s, 3H); LCMS (ESI⁺), M+H⁺: 306.

### Example 98

### 6-(1-(4-Fluorophenyl)-3-methyl-1H-pyrazol-5-yl)-2H-benzo[b][1,4]oxazin-3(4H)-one

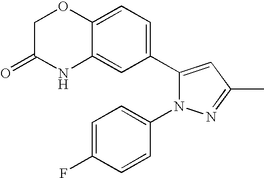

According to the method of Example 96, 6-(3-hydroxybut-2-enoyl)-2H-benzo[b][1,4]oxazin-3(4H)-one (150 mg, 0.643 mmol), (4-fluorophenyl)hydrazine hydrochloride (110 mg, 0.675 mmol) and triethylamine (179 μL, 1.29 mmol) were reacted to give, after preparative TLC on silica gel (50% EtOAc in petroleum ether), the title compound as an off-white solid (6.4 mg, 3%).

¹H-NMR (400 MHz, CDCl₃) δ: 8.35 (s, 1H), 7.24 (m, 2H), 7.03 (m, 2H), 6.89 (d, J=8.2 Hz, 1H), 6.78 (dd, J=8.2, 1.9 Hz, 1H), 6.64 (d, J=1.9 Hz, 2H), 6.25 (s, 2H), 4.63 (s, 3H); LCMS (ESI⁺), M+H⁺: 324.

### Example 99

### 6-(3-Methyl-1-(4-nitrophenyl)-1H-pyrazol-5-yl)-2H-benzo[b][1,4]oxazin-3(4H)-one

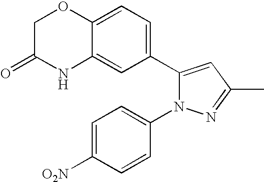

According to the method of Example 96, 6-(3-hydroxybut-2-enoyl)-2H-benzo[b][1,4]oxazin-3(4H)-one (150 mg, 0.643 mmol) and (4-nitrophenyl)hydrazine (103 mg, 0.675 mmol) were reacted to give the title compound as an orange solid (36 mg, 17%).

¹H-NMR (400 MHz, CDCl₃) δ: 8.20 (d, J=8.9 Hz, 2H), 7.45 (d, J=9.4 Hz, 2H), 6.95 (m, 3H), 6.30 (s, 1H), 4.67 (s, 2H), 2.38 (s, 3H); LCMS (ESI⁺), M+H⁺: 351.

### Example 100

### 6-(1-(4-Fluorophenyl)-3-(perfluoroethyl)-1H-pyrazol-5-yl)-2H-benzo[b][1,4]oxazin-3(4H)-one

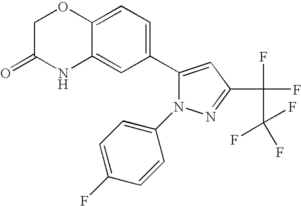

### 6-(4,4,5,5,5-Pentafluoro-3-hydroxypent-2-enoyl)-2H-benzo[b][1,4]oxazin-3(4H)-one

According to the method of Example 71, 6-acetyl-2H-benzo[b][1,4]oxazin-3(4H)-one (500 mg, 2.62 mmol) and ethyl 2,2,3,3,3-pentafluoropropanoate (1.55 mL, 10.5 mmol) were reacted to give the title compound as a solid (763 mg, 87%).

¹H-NMR (400 MHz, DMSO-d₆) δ: 10.71 (s, 1H), 7.38 (d, J=2.0 Hz, 1H), 7.34 (dd, J=8.2, 2.0 Hz, 1H), 6.91 (d, J=8.2 Hz, 1H), 5.80 (s, 1H), 4.59 (s, 2H).

### 6-(1-(4-Fluorophenyl)-3-(perfluoroethyl)-1H-pyrazol-5-yl)-2H-benzo[b][1,4]oxazin-3(4H)-one

According to the method of Example 71, 6-(4,4,5,5,5-pentafluoro-3-hydroxypent-2-enoyl)-2H-benzo[b][1,4]oxazin-3(4H)-one (0.763 g, 2.26 mmol) and (4-fluorophenyl)hydrazine hydrochloride (386 mg, 2.38 mmol) were reacted to give the title compound as a tan solid (627 mg, 65%).

¹H-NMR (400 MHz, CDCl₃) δ: 8.26 (s, 1H), 7.31 (m, 2H), 7.09 (m, 2H), 6.93 (d, J=8.2 Hz 1H), 6.81 (dd, J=8.2, 2.0 Hz 1H), 6.72 (s, 1H), 6.66 (d, J=2.0 Hz, 1H), 4.66 (s, 2H);

LCMS (ESI⁺), M+H⁺: 428.

### Example 101

### 6-(3-(Difluoromethyl)-1-phenyl-1H-pyrazol-5-yl)-2H-benzo[b][1,4]oxazin-3(4H)-one

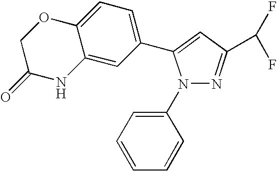

### 6-(4,4-Difluoro-3-hydroxybut-2-enoyl)-2H-benzo[b][1,4]oxazin-3(4H)-one

According to the method of Example 71, 6-acetyl-2H-benzo[b][1,4]oxazin-3(4H)-one (500 mg, 2.62 mmol) and ethyl 2,2-difluoroacetate (1.10 mL, 10.5 mmol) gave the title compound as a tan solid (580 mg, 82%).

¹H-NMR (400 MHz, CDCl₃) δ: 8.08 (s, 1H), 7.60 (dd, J=8.6, 2.3 Hz, 1H), 7.44 (d, J=2.3 1H), 7.06 (d, J=8.6 Hz, 1H), 6.49 (s, 1H), 6.15-5.89 (t, J=54.3 Hz, 1H), 4.73 (s, 2H);

LCMS (ESI⁻), M−H⁺: 268.

### 6-(3-(Difluoromethyl)-1-phenyl-1H-pyrazol-5-yl)-2H-benzo[b][1,4]oxazin-3(4H)-one

A stirred solution of 6-(4,4-difluoro-3-hydroxybut-2-enoyl)-2H-benzo[b][1,4]oxazin-3(4H)-one (580 mg, 2.16 mol) and phenylhydrazine (233 μL, 2.37 mmol) in isopropyl alcohol was refluxed overnight in the presence of acetic acid (49 μL, 0.862 mmol). The resulting solids were filtered to yield a mixture of regioisomers. Purification by preparative HPLC (YMC ODS-AQ 250×20 mm S-15 um S/N #208722; 68-95% acetonitrile with 0.05% TFA) gave the title compound as a tan solid (36 mg, 5%).

¹H-NMR (400 MHz, CD₃CN) δ: 8.53 (s, 1H), 7.42 (m, 3H), 7.29 (m, 2H), 6.88 (d, J=8.6 Hz, 1H), 6.85 (t, J=54.8 Hz, 1H), 6.8 (dd, J=8.6, 1.9 Hz, 1H), 6.75 (m, 1H), 6.74 (d, J=1.9 Hz H), 4.53 (s, 2H); LCMS (ESI⁺), M+H⁺: 342.

### Example 102

### Ethyl 1-(4-fluorophenyl)-5-(3-oxo-3,4-dihydro-2H-benzo[b][1,4]oxazin-6-yl)-1H-pyrazole-3-carboxylate

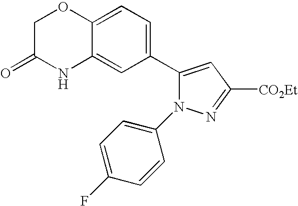

### Ethyl 2,4-dioxo-4-(3-oxo-3,4-dihydro-2H-benzo[b][1,4]oxazin-6-yl)butanoate

According to the method of Example 71, 6-acetyl-2H-benzo[b][1,4]oxazin-3(4H)-one (1.0 g, 5.23 mmol) and diethyl oxalate (1.43 mL, 10.5 mmol) were reacted to give the title compound as a yellow solid (1.45 g, 95%).

LCMS (ESI⁻), M−H⁻: 290.

### Ethyl 1-(4-fluorophenyl)-5-(3-oxo-3,4-dihydro-2H-benzo[b][1,4]oxazin-6-yl)-1H-pyrazole-3-carboxylate

According to the method of Example 71, ethyl 2,4-dioxo-4-(3-oxo-3,4-dihydro-2H-benzo[b][1,4]oxazin-6-yl)butanoate (7.78 g, 26.7 mmol) and (4-fluorophenyl)hydrazine hydrochloride (4.56 g, 28.0 mmol) were reacted to give the title compound as a tan solid (8.14 g, 80%).

¹H-NMR (400 MHz, DMSO-d₆) δ: 10.76 (s, 1H), 7.40 (m, 2H), 7.33 (m, 2H), 7.02 (s, 1H), 6.93 (d, J=8.2 Hz, 1H), 6.80 (dd, J=8.2, 2.0 Hz, 1H), 6.76 (d, J=2.0 Hz, 1H), 4.60 (s, 2H), 4.33 (q, J=7.2 Hz, 2H), 1.32 (t, J=7.2 Hz, 3H);

LCMS (ESI⁺), M+H⁺: 382.

### Example 103

### 1-(4-Fluorophenyl)-N,N-dimethyl-5-(3-oxo-3,4-dihydro-2H-benzo[b][1,4]oxazin-6-yl)-1H-pyrazole-3-carboxamide

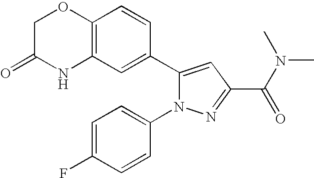

### 1-(4-Fluorophenyl)-5-(3-oxo-3,4-dihydro-2H-benzo[b][1,4]oxazin-6-yl)-1H-pyrazole-3-carboxylic acid

Ethyl 1-(4-fluorophenyl)-5-(3-oxo-3,4-dihydro-2H-benzo[b][1,4]oxazin-6-yl)-1H-pyrazole-3-carboxylate (1.26 g, 3.30 mmol) was dissolved in THF (25.0 mL), NaOH (8.26 mL, 8.26 mmol) was added, and the reaction mixture was heated under reflux overnight. Upon cooling, 1N HCl was added until the mixture was acidic, and the solids were filtered and recrystallized from ethanol to provide the title compound as a white solid (877 mg, 75%).

¹H-NMR (400 MHz, DMSO-d₆) δ: 12.98 (s, 1H), 10.76 (s, 1H), 7.38 (m, 2H), 7.32 (m, 2H), 6.96 (s, 1H), 6.93 (d, J=8.2 Hz, 1H), 6.79 (dd, J=8.2, 2.0 Hz, 1H), 6.76 (d, J=2.0 Hz, 1H), 4.60 (s, 2H); LCMS (ESI⁺), M+H⁺: 354.

### 1-(4-Fluorophenyl)-N,N-dimethyl-5-(3-oxo-3,4-dihydro-2H-benzo[b][1,4]oxazin-6-yl)-1H-pyrazole-3-carboxamide

1-(4-Fluorophenyl)-5-(3-oxo-3,4-dihydro-2H-benzo[b][1,4]oxazin-6-yl)-1H-pyrazole-3-carboxylic acid (52 mg, 0.147 mmol) was dissolved in DMF (2.0 mL), and HOBt monohydrate (24.8 mg, 0.162 mmol) and EDCI (33.9 mg, 0.177 mmol) were added. After stirring for 30 min, dimethylamine (77 μL, 0.155 mmol) was added and the reaction stirred over the weekend. The reaction mixture was diluted with water and extracted with EtOAc. The organic extract was washed with 10% LiCl solution and brine, dried (MgSO₄) and concentrated in vacuo. The residue was triturated with EtOAc and filtered to give the title compound as a white solid (24 mg, 44%).

¹H-NMR (400 MHz, CD₃CN) δ: 8.55 (s, 1H), 7.34 (m, 2H), 7.15 (m, 2H), 6.89 (d, J=8.2 Hz, 1H), 6.86 (dd, J=8.2, 2.0 Hz, 1H), 6.76 (s, 1H), 6.71 (d, J=2.0 Hz, 1H), 4.53 (s, 2H), 3.13 (s, 3H), 3.04 (s, 3H); LCMS (ESI⁺), M+H⁺: 381.

### Example 104

### 1-(4-Fluorophenyl)-5-(3-oxo-3,4-dihydro-2H-benzo[b][1,4]oxazin-6-yl)-1H-pyrazole-3-carbonitrile

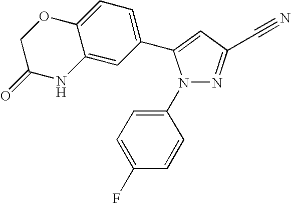

### 1-(4-Fluorophenyl)-5-(3-oxo-3,4-dihydro-2H-benzo[b][1,4]oxazin-6-yl)-1H-pyrazole-3-carboxamide

According to the method of Example 103, 1-(4-fluorophenyl)-5-(3-oxo-3,4-dihydro-2H-benzo[b][1,4]oxazin-6-yl)-1H-pyrazole-3-carboxylic acid (170 mg, 0.481 mmol) and ammonia (253 μL, 2.0 M in MeOH, 0.505 mmol) gave the title compound as a white solid (130 mg, 76%).

1H-NMR (400 MHz, DMSO-d₆) δ: 10.76 (s, 1H), 7.69 (s, 1H), 7.39 (m, 3H), 7.32 (t, J=8.9 Hz, 2H), 6.93 (d, J=8.9 Hz, 1H), 6.89 (s, 1H), 6.77 (m, 2H), 4.60 (s, 2H); LCMS (ESI⁻), M−H⁻: 351.

### 1-(4-Fluorophenyl)-5-(3-oxo-3,4-dihydro-2H-benzo[b][1,4]oxazin-6-yl)-1H-pyrazole-3-carbonitrile

To a stirred solution of oxalyl chloride (12 μL, 0.142 mmol) in DMF (1.0 mL) was added a solution of 1-(4-fluorophenyl)-5-(3-oxo-3,4-dihydro-2H-benzo[b][1,4]oxazin-6-yl)-1H-pyrazole-3-carboxamide (50 mg, 0.142 mmol) in DMF (1.0 mL) at 0° C. and the reaction mixture was stirred for 15 min at 0° C. The reaction mixture was quenched with pyridine and poured into 1N HCl and the mixture was extracted with EtOAc. The organic extract was washed with 1N HCl and brine, dried (MgSO₄) and concentrated in vacuo. Purification of the residue on silica gel (0-10% EtOAc in DCM) gave the title compound as a yellow solid (9.4 mg, 20%).

¹H-NMR (400 MHz, DMSO-d₆) δ: 10.80 (s, 1H), 7.44 (m, 2H), 7.34 (m, 3H), 6.95 (d, J=8.6 Hz, 1H), 6.80 (dd, J=8.6, 2.0 Hz, 1H), 6.75 (d, J=2.0 Hz, 1H), 4.60 (s, 2H); LCMS (ESI⁺), M+H⁺: 335.

### Example 105

### 6-(4-Bromo-1-phenyl-3-(trifluoromethyl)-1H-pyrazol-5-yl)-2H-benzo[b][1,4]oxazin-3(4H)-one

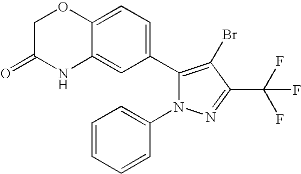

To a stirred solution of 6-(1-phenyl-3-(trifluoromethyl)-1H-pyrazol-5-yl)-2H-benzo[b][1,4]oxazin-3(4H)-one (50 mg, 0.14 mmol) in DMF (1.0 mL) was added NBS (25 mg, 0.14 mmol) at 0° C. The reaction mixture was allowed to stir overnight at room temperature. Additional NBS (25 mg, 0.14 mmol) was added and stirring was continued overnight. The reaction mixture was partitioned between water and EtOAc, and the organic layer was washed with brine, dried (MgSO₄) and concentrated in vacuo. Purification of the residue by flash chromatography on silica gel (10% EtOAc in DCM) followed by preparative TLC (50% EtOAc in petroleum ether) gave the title compound as a white solid (7.4 mg, 12%).

¹H-NMR (400 MHz, CDCl₃) δ: 7.58 (s, 1H), 7.36 (m, 3H), 7.24 (m, 2H), 6.98 (m, 1H), 6.86 (dd, J=8.6, 2.0 Hz, 1H), 6.70 (d, J=2.0 Hz, 1H), 4.67 (s, 2H); LCMS (ESI⁺), M+H⁺: 440.

### Example 106

### 6-(2-Phenylimidazo[1,2-a]pyridin-3-yl)-2H-benzo[b][1,4]oxazin-3(4H)-one

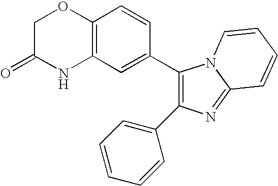

A stirred solution of 6-(2-bromo-2-phenylacetyl)-2H-benzo[b][1,4]oxazin-3(4H)-one (226 mg, 0.653 mmol), pyridine-2-amine (67.6 mg, 0.718 mmol) and p-toluenesulfonic acid hydrate (12.4 mg, 0.065 mmol) in CH₃CN (3.30 mL) was heated under reflux overnight (16 h) and then concentrated in vacuo. Trituration of the residue with DCM gave the title compound as a yellow powder (54 mg, 24%).

¹H-NMR (400 MHz, DMSO-d₆) δ: 10.89 (bs, 1H), 8.24 (d, J=5.8 Hz, 1H), 7.88 (d, J=8.2 Hz, 1H), 7.76 (bs, 1H), 7.63 (m, 3H), 7.56 (m, 2H), 7.25 (bs, 1H), 7.18 (s, 1H), 7.01 (dd, J=8.2, 2.0 Hz, 1H), 6.97 (d, J=8.2 Hz, 1H), 4.62 (s, 2H);

LCMS (ESI⁺), M+H⁺: 342.

### Example 107

### 6-(1-(4-Fluoro-2-methylphenyl)-3-(trifluoromethyl)-1H-pyrazol-5-yl)-2H-benzo[b][1,4]oxazin-3(4H)-one

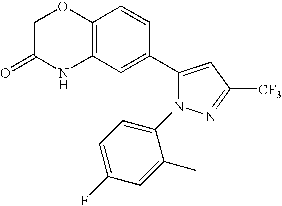

### 1-(4-Fluoro-2-methylphenyl)hydrazine hydrochloride

To a solution of 4-fluoro-2-methylaniline (125 g, 1.00 mol) in conc. HCl (1000 ml) was added NaNO₂ (137 g, 2.00 mol) as a solid with cooling and the mixture was stirred at 0° C. for 2 hr. To the mixture was added SnCl₂ (474 g, 2.50 mol) as a solid at 0° C. The reaction mixture was stirred at 0° C. for 3 hr and room temperature overnight, and poured into a separatory funnel and washed with ether (250 ml). The aqueous layer was slowly and carefully added to aqueous NaOH under ice cooling to basify the solution. The basic aqueous layer was extracted with ethyl acetate, and the organic layer was dried and concentrated to give 1-(4-fluoro-2-methylphenyl)hydrazine, that solidified upon standing. The residue was dissolved with a minimal amount of ether and precipitated with 4N HCl/dioxane to afford the title compound as a white solid (85.0 g, 48%). The compound was used in subsequent reactions without further purification.

LCMS (ESI⁺), M+H⁺: 141.

### 6-(1-(4-Fluoro-2-methylphenyl)-3-(trifluoromethyl)-1H-pyrazol-5-yl)-2H-benzo[b][1,4]oxazin-3(4H)-one

To a slurry of 1-(4-fluoro-2-methylphenyl)hydrazine hydrochloride (29.0 g, 164 mmol) in isopropanol (350 ml) were added triethylamine (16.6 g, 22.9 ml, 164 mmol) and then trifluoroacetic acid (12.64 ml, 164.1 mmol). To this solution was then added 4,4,4-trifluoro-1-(3-oxo-3,4-dihydro-2H-benzo[b][1,4]oxazin-6-yl)butane-1,3-dione (47.1 g, 164 mmol) and the resulting solution was heated at 80° C. for 3 hr, monitoring by LCMS. The reaction mixture was complete after 3 hr. The reaction mixture was poured into water (1.0 l) and the brown precipitate was collected by filtration. The precipitate was purified by chromatography, eluting with ethyl acetate/hexane. The product fractions were collected and concentrated to afford the title compound as a white solid (31.4 g, 54%).

¹H-NMR (400 MHz, DMSO-d₆) δ: 10.78 (s, 1H), 7.43 (dd, J=8.7, 5.4 Hz, 1H), 7.28 (dd, J=9.8, 3.0 Hz, 1H), 7.11-7.23 (m, 1H), 7.13 (s, 1H), 6.92 (d, J=8.1, 1H), 6.82 (dd, J=8.4, 2.1 Hz, 1H), 6.70 (d, 2.1 Hz, 1H), 4.58 (s, 2H), 1.90 (s, 3H); LCMS (ESI⁻), M−H⁺: 390.

### Example 108

### 8-Fluoro-6-(1-(4-fluoro-2-methylphenyl)-3-(trifluoromethyl)-1H-pyrazol-5-yl)-2H-benzo[b][1,4]oxazin-3(4H)-one

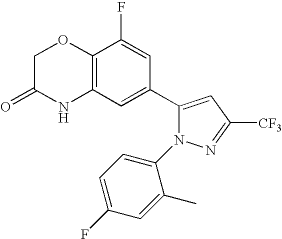

### Methyl 2-(4-bromo-2-fluoro-6-nitrophenoxy)acetate

A mixture of 4-bromo-2-fluoro-6-nitrophenol (216 g, 917 mmol), methyl 2-bromoacetate (104 ml, 1.10 mol) and K₂CO₃ (633 g, 4.58 mol) in DMF (500 ml) was heated at 65° C. overnight. The reaction mixture was poured into water and the off-white precipitate was collected by filtration and to give the title compound (282 g, 99%). This compound was taken onto the next step as is.

LCMS (ESI⁻), M−H⁺: 307.

### 6-Bromo-8-fluoro-2H-benzo[b][1,4]oxazin-3(4H)-one

To a solution of methyl 2-(4-bromo-2-fluoro-6-nitrophenoxy)acetate (282.0 g, 915.41 mmol) in acetic acid (1.5 L) was slowly added Zn dust (209.51 g, 3203.9 mmol) to avoid excessive exothermic reaction. The reaction mixture was heated at 100° C. overnight, following the reaction by LCMS. The reaction mixture was filtered through a paper filter. The solid filter cake was heated with DMF, and the mixture was filtered through a paper filter. The combined filtrates were poured into water. The precipitate was collected by filtration and collected to give the title compound as a white solid (130 g, 57%).

LCMS (ESI⁻), M−H⁺: 244.

### 6-Acetyl-8-fluoro-2H-benzo[b][1,4]oxazin-3(4H)-one

A solution of 6-bromo-8-fluoro-2H-benzo[b][1,4]oxazin-3(4H)-one (93.7 g, 381 mmol), 4-(vinyloxy)butan-1-ol (156 ml, 1.26 mol), trans-dichlorobis(tri-o-tolylphosphine) palladium II (8.98 g, 11.4 mmol) and K₂CO₃ (105 g, 762 mmol) in a mixed solvent of DMF (635 ml) and H₂O (38.1 ml) was degassed with nitrogen and heated at 80° C. overnight. The mixture was poured into 2N HCl and stirred for 1 hr, and then extracted with DCM. The organic layers were combined, dried and concentrated. Flash chromatography of the residue on silica gel eluting with ethyl acetate/hexane afforded the title compound as a tan solid (58.0 g, 72%).

LCMS (ESI⁻), M−H⁺: 209.

### 4,4,4-Trifluoro-1-(8-fluoro-3-oxo-3,4-dihydro-2H-benzo[b][1,4]oxazin-6-yl)butane-1,3-dione

To a slurry of 60% NaH (44.36 g, 1109 mmol) in THF (4.0 L) was slowly added ethyl 2,2,2-trifluoroacetate (145.9 ml, 1109 mmol). 6-Acetyl-8-fluoro-2H-benzo[b][1,4]oxazin-3(4H)-one (58 g, 277.3 mmol) was slowly added as a solid, and then 2,4-dibenzo-18-crown-6 (Aldrich, CAS 14262-61-4, 1.599 g, 4.437 mmol) and ethanol (1.5 ml, absolute) were added. The resulting mixture was heated at 65° C. for 2 hr, poured into 1N HCl and extracted with ethyl acetate. The organic layer was washed with water, dried and concentrated. The residue was triturated with ether to give the title compound as a tan solid (33.0 g, 39%).

LCMS (ESI⁻), M−H⁺: 304.

### 8-Fluoro-6-(1-(4-fluoro-2-methylphenyl)-3-(trifluoromethyl)-1H-pyrazol-5-yl)-2H-benzo[b][1,4]oxazin-3(4H)-one

To a slurry of 1-(4-fluoro-2-methylphenyl)hydrazine hydrochloride (19.1 g, 108 mmol) in i-PrOH (500 ml) was added triethylamine (15.1 ml, 108 mmol). To this solution were added trifluoroacetic acid (8.33 ml, 108 mmol) and 4,4,4-trifluoro-1-(8-fluoro-3-oxo-3,4-dihydro-2H-benzo[b][1,4]oxazin-6-yl)butane-1,3-dione (33.0 g, 108.1 mmol). The resulting mixture was heated at 80° C. for 3 hr and poured into water. The precipitate was collected by filtration and purified by chromatography using a Biotage Flash 75L, eluting with ethyl acetate/hexane to afford the title compound as a white solid (35.2 g, 79%).

¹H-NMR (400 MHz, DMSO-d₆) δ: 10.98 (s, 1H), 7.46 (dd, J=9.0, 5.5 Hz, 1H), 7.30 (dd, J=9.5, 2.9 Hz, 1H), 7.23 (s, 1H), 7.20 (td, J=8.4, 2.9 Hz, 1H), 6.91 (dd, J=11.3, 2.0 Hz, 1H), 6.47 (m, 1H), 4.67 (s, 2H), 1.91 (s, 3H); LCMS (ESI⁻), M−H⁺: 408.

### Example 109

### 8-Chloro-6-(1-(4-fluoro-2-methylphenyl)-3-(trifluoromethyl)-1H-pyrazol-5-yl)-2H-benzo[b][1,4]oxazin-3(4H)-one

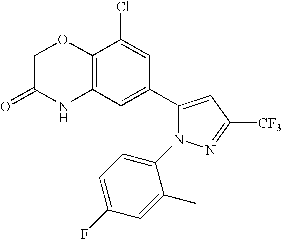

### 4-Bromo-2-chloro-6-nitrophenol

To a solution of 4-bromo-2-chlorophenol (400 g, 1.93 mol) in acetic acid (2.0 l) at room temperature was added nitric acid (70%, 231 ml, 3.86 mol) slowly and the resulting solution was stirred at room temperature overnight. The reaction mixture was poured into water and the yellow precipitate was collected by filtration to afford the title compound (412 g, 84%).

LCMS (ESI⁻), M−H⁺: 252.

### Methyl 2-(4-bromo-2-chloro-6-nitrophenoxy)acetate

A mixture of 4-bromo-2-chloro-6-nitrophenol (412 g, 1.63 mol), methyl 2-bromoacetate (185 ml, 1.96 mol) and K₂CO₃ (1.13 kg, 8.16 mol) in DMF (800 ml) was heated at 70° C. overnight. The reaction mixture was poured into water, and the precipitate was collected by filtration to give the title compound as yellow solid (230 g, 43%).

LCMS (ESI⁻), M−H⁺: 323.

### 6-Bromo-8-chloro-2H-benzo[b][1,4]oxazin-3(4H)-one

To a solution of methyl 2-(4-bromo-2-chloro-6-nitrophenoxy)acetate (230 g, 710 mmol) in acetic acid was slowly added Zn dust (163 g, 2.49 mol) to avoid an excessively exothermic reaction. Upon completion of the addition, the reaction mixture was heated at 100° C. for 45 min, at which point the reaction mixture was filtered through a Buchner funnel equipped with a paper filter. The filter cake was added to DMF and this mixture was heated to 80° C. and stirred at this temperature for 30 min. The hot mixture was filtered through a paper. The combined filtrates were poured into water and the white precipitate was collected by filtration to afford the title compound (181 g, 97%).

LCMS (ESI⁻), M−H⁺: 261.

### 6-Acetyl-8-chloro-2H-benzo[b][1,4]oxazin-3(4H)-one

A mixture of 6-bromo-8-chloro-2H-benzo[b][1,4]oxazin-3(4H)-one (131 g, 499 mmol), 4-(vinyloxy)butan-1-ol (204 ml, 1.65 mol), Cl₂Pd(P-(o-tol)₃)₂ (19.6 g, 25.0 mmol) and K₂CO₃ (207 g, 1.50 mol) in a mixed solvent of DMF (832 ml) and H₂O (50.0 ml) was degassed by bubbling with nitrogen and the resulting mixture was heated at 80° C. overnight. The mixture was poured into 2N-HCl and stirred for 1 hr. The mixture was extracted with ethyl acetate, and the organic layer was washed with water, dried and concentrated. The residue was purified by column chromatography, eluting with ethyl acetate/hexane to afford the title compound (38.0 g, 34%).

LCMS (ESI⁻), M−H⁺: 224.

### 1-(8-Chloro-3-oxo-3,4-dihydro-2H-benzo[b][1,4]oxazin-6-yl)-4,4,4-trifluorobutane-1,3-dione

To a slurry of 60% NaH (27.0 g, 6734 mmol) in THF was slowly added ethyl 2,2,2-trifluoroacetate (80.4 ml, 676 mmol). To this mixture was added 6-acetyl-8-chloro-2H-benzo[b][1,4]oxazin-3(4H)-one (38.0 g, 168 mmol) as a solid, and then dibenzo-18-crown-6 (0.97 g, 2.69 mmol) and ethanol (1.00 ml, absolute) were added. The reaction mixture was heated at 65° C. for 2 hr, poured into 1N-HCl and extracted with ethyl acetate. The organic layer was washed with water, dried and concentrated. The residue was triturated with ether/petroleum ether to give the title compound as a tan solid (35.0 g, 65%).

LCMS (ESI⁻), M−H⁺: 320.

### 8-Chloro-6-(1-(4-fluoro-2-methylphenyl)-3-(trifluoromethyl)-1H-pyrazol-5-yl)-2H-benzo[b][1,4]oxazin-3(4H)-one

To a slurry of 1-(4-fluoro-2-methylphenyl)hydrazine hydrochloride (19.2 g, 109 mmol) in i-PrOH (250 ml) was added triethylamine (15.2 ml, 109 mmol) followed by trifluoroacetic acid (8.4 ml, 113 mmol) and the resulting mixture was stirred for 5 min. To the mixture was added 1-(8-Chloro-3-oxo-3,4-dihydro-2H-benzo[b][1,4]oxazin-6-yl)-4,4,4-trifluorobutane-1,3-dione (35.0 g, 109 mmol). The mixture was heated at 80° C. for 3 hr, diluted with ethyl acetate and washed successively with water, 1N-HCl and brine, dried and concentrated to give crude material. The crude material was purified by column chromatography, eluting with ethyl acetate/hexane to afford the title compound (19.6 g, 43%).

¹H-NMR (400 MHz, DMSO-d₆) δ: 10.96 (s, 1H), 7.45 (dd, J=8.6, 5.5 Hz, 1H), 7.30 (dd, J=9.8, 3.0 Hz, 1H), 7.25 (s, 1H), 7.20 (td, J=8.5, 2.7 Hz, 1H), 7.07 (d, J=2.3 Hz, 1H), 6.59 (d, J=2.0 Hz, 1H), 4.71 (s, 2H), 1.91 (s, 3H);

LCMS (ESI⁻), M−H⁺: 424.

### Example 110

### 6-(1-(4-Fluoro-2-methylphenyl)-3-(trifluoromethyl)-1H-pyrazol-5-yl)-8-methyl-2H-benzo[b][1,4]oxazin-3(4H)-one

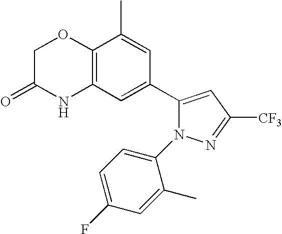

### 4-Hydroxy-3-methyl-5-nitroacetophenone

To a solution of 4-hydroxy-3-methylacetophenone (100 g, 666 mmol) in acetic acid (444 ml) was added nitric acid (70%, 31.0 ml, 732 mmol) at room temperature. The resulting solution was stirred at room temperature for 24 hr. The reaction mixture was poured into water and the white solid precipitate was collected by vacuum filtration to afford the title compound (77.0 g, 59%).

¹H-NMR (400 MHz, acetone-d₆) δ: 8.57 (d, J=2.3 Hz, 1H), 8.18 (m, 1H), 2.62 (s, 3H), 2.38 (s, 3H).

### Methyl 2-(4-acetyl-2-methyl-6-nitrophenoxy)acetate

A mixture of 4-hydroxy-3-methyl-5-nitroacetophenone (77.0 g, 395 mmol), methyl 2-bromoacetate (90.5 g, 592 mmol), K₂CO₃ (164 g, 1.18 mol) and DMF (800 ml) was stirred at room temperature overnight. The reaction mixture was poured into water, and the white precipitate was collected by vacuum filtration to afford the title compound (99.0 g, 94%).

¹H-NMR (400 MHz, CD₃OD) δ: 8.26 (d, J=2.3 Hz, 1H), 8.14 (m, 1H), 4.85 (s, 2H), 3.79 (s, 3H), 2.61 (s, 3H), 2.46 (s, 3H).

### 6-Acetyl-8-methyl-2H-benzo[b][1,4]oxazin-3(4H)-one

To a solution of methyl 2-(4-acetyl-2-methyl-6-nitrophenoxy)acetate (99.0 g, 370 mmol) in acetic acid (750 ml) was slowly added Zn dust (115.08 g, 1759.9 mmol) to avoid an excessively exothermic reaction. Upon completion of the addition, the reaction mixture was heated at 100° C. for 45 min, at which point the hot reaction mixture was filtered through a Buchner funnel equipped with a paper filter. The filter cake was added to DMF and this mixture was heated to 80° C. and stirred at this temperature for 30 min. The hot mixture was filtered through a paper filter. The filtrates were poured into water and the white precipitate was collected by filtration to afford the title compound (72.0 g, 95%).

LCMS (ESI⁻), M−H⁺: 204.

### 4,4,4-Trifluoro-1-(8-methyl-3-oxo-3,4-dihydro-2H-Benzo[b][1,4]oxazin-6-yl)butane-1,3-dione

To a slurry of 60% NaH (56.1 g, 1.40 mol) in THF (4.6 L) was slowly added ethyl 2,2,2-trifluoroacetate (167.4 ml, 1.41 mol). To this mixture was added 6-acetyl-8-methyl-2H-benzo[b][1,4]oxazin-3(4H)-one (72.0 g, 351 mmol) as a solid, and then dibenzo-18-crown-6 (0.97 g, 2.69 mmol) and ethanol (1.00 ml, absolute) were added. The resulting mixture was heated at 65° C. for 2 hr, poured into 1N-HCl and extracted with ethyl acetate. The organic layer was washed with water, dried and concentrated. The residue was triturated with ether/petroleum ether to give the title compound as an off-white solid (37.20 g, 35%).

LCMS (ESI⁻), M−H⁺: 300.

### 6-(1-(4-Fluoro-2-methylphenyl)-3-(trifluoromethyl)-1H-pyrazol-5-yl)-8-methyl-2H-benzo[b][1,4]oxazin-3(4H)-one

To a solution of 1-(4-fluoro-2-methylphenyl)hydrazine hydrochloride (23.99 g, 136 mmol) in i-PrOH (617.5 ml) were added triethylamine (19.0 ml, 136 mmol) then trifluoroacetic acid (19.0 ml, 256 mmol) and the resulting solution was stirred for 5 min. To this solution was then added 4,4,4-trifluoro-1-(8-methyl-3-oxo-3,4-dihydro-2H-benzo[b][1,4]oxazin-6-yl)butane-1,3-dione (37.20 g, 123.5 mmol) and the solution was heated at 80° C. for 3 hr. The reaction mixture was diluted with ethyl acetate, washed successively with water, 1N-HCl and brine, dried and concentrated to give crude material, which was purified by column chromatography, eluting with ethyl acetate/hexane to afford the title compound as a white solid (22.5 g, 45%).

¹H-NMR (400 MHz, CDCl₃) δ: 9.00 (s, 1H), 7.26 (m, 1H), 6.98 (m, 2H), 6.74 (s, 1H), 6.70 (m, 1H), 6.39 (d, J=2.0 Hz, 1H), 4.64 (s, 2H), 2.15 (s, 3H), 1.96 (s, 3H); LCMS (ESI⁻), M−H⁺: 404.

### Preparation 43

### 3-(4-Fluorophenyl)-2-iodoacrylaldehyde

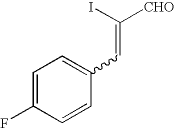

A suspension of 4-fluorobenzaldehyde (9.40 g, 75.8 mmol) and formylmethylene triphenylphosphorane (30.0 g, 98.6 mmol) in toluene (150 ml) was stirred at 70° C. for 12 hr. The reaction mixture was treated with EtOAc and H₂O. The organic layer was separated, washed with brine, dried over Na₂SO₄ and concentrated in vacuo. The residue was purified by column chromatography to give unsaturated aldehyde (8.04 g, 71%). To a solution of the resultant aldehyde (8.04 g, 53.5 mmol) in a mixed solvent of pyridine (100 ml) and dichloromethane (50 ml) was added iodine monochloride (17.4 g, 107 mmol) at 0° C. After stirring for 5 hr at 0° C., the reaction mixture was quenched with aqueous Na₂S₂O₃ solution and treated with EtOAc. The organic layer was separated, washed with brine, dried over Na₂SO₄ and concentrated in vacuo. The residue was purified by column chromatography to give the title compound (10.01 g, 68%).

¹H-NMR (300 MHz, DMSO-d₆) δ: 7.43 (2H, t, J=8.5 Hz), 8.13 (2H, dd, J=8.5, 5.5 Hz), 8.54 (1H, s), 8.84 (1H, s).

### Preparation 44

### 3-(4-Fluorophenyl)-2-(3-oxo-3,4-dihydro-2H-1,4-benzoxazin-6-yl)acrylaldehyde

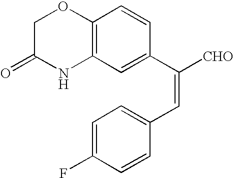

A mixture of 3-(4-fluorophenyl)-2-iodoacrylaldehyde (2.2 g), 6-(4,4,5,5-tetramethyl-1,3,2-dioxaborolan-2-yl)-2H-1,4-benzoxazin-3(4H)-one (2.19 g), [1,1-bis(diphenylphosphino)ferrocene]dichloropalladium(II) dichloromethane adduct (1.46 g), 2M Cs₂CO₃ (13 ml) and THF (80 ml) was stirred under reflux for 12 hr, and then treated with ethyl acetate and water. The organic layer was separated, dried over MgSO₄ and concentrated in vacuo. The residue was chromatographed on silica gel with hexane/ethyl acetate as an eluent to give the title compound (1.25 g).

¹H-NMR (300 MHz, DMSO-d₆) δ: 4.63 (2H, s), 6.65-6.69 (2H, m), 6.98-7.01 (1H, m), 7.16-7.22 (2H, m), 7.32-7.36 (2H, m), 7.66 (1H, s), 9.70 (1H, s), 10.71 (1H, s); MS (ESI) m/z: 268 (M+1).

### Example 111

### 6-[2-Amino-6-(4-fluorophenyl)-6H-1,3-thiazin-5-yl]-2H-1,4-benzoxazin-3(4H)-one

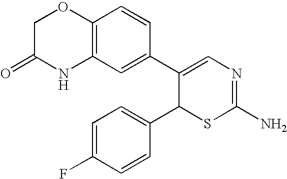

A mixture of 3-(4-fluorophenyl)-2-(3-oxo-3,4-dihydro-2H-1,4-benzoxazin-6-yl)-acrylaldehyde (1.20 g), thiourea (0.37 g), 1,4-dioxane (40 ml), water (8 ml) and conc. HCl (4 ml) was stirred at 100° C. for 12 hr, and then treated with THF and saturated aqueous NaHCO₃. The resultant precipitate in the organic layer was collected by filtration to give the title compound (1.2 g).

¹H-NMR (300 MHz, DMSO-d₆) δ: 4.50 (2H, s), 5.22 (1H, s), 6.81-6.96 (5H, m), 7.09-7.17 (3H, m), 7.26-7.30 (2H, m), 10.61 (1H, s); MS (ESI) m/z: 356 (M+1).

### Example 112

### 6-[7-(4-Fluorophenyl)-7H-imidazo[2,1-b][1,3]thiazin-6-yl]-2H-1,4-benzoxazin-3(4H)-one hydrochloride

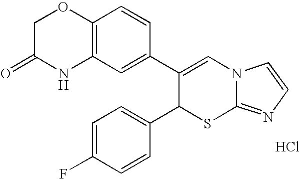

A mixture of 6-[2-amino-6-(4-fluorophenyl)-6H-1,3-thiazin-5-yl]-2H-1,4-benzoxazin-3(4H)-one (300 mg) and 45% chloroacetaldehyde solution (1.2 g) in dimethoxyethane/ethanol (1/1, 20 ml) was stirred at 100° C. for 12 hr. After cooling to room temperature, the precipitated crystals were collected by filtration to give the title compound (127 mg).

¹H-NMR (300 MHz, DMSO-d₆) δ: 4.58 (2H, s), 5.78 (1H, s), 6.91-6.92 (3H, m), 6.98-7.01 (1H, m), 7.06-7.10 (1H, m), 7.14-7.20 (2H, m), 7.29-7.37 (1H, m), 7.79-7.80 (1H, m), 7.93 (1H, s), 10.83 (1H, s); MS (ESI) m/z 380 (M+1).

### Preparation 45

### 6-Bromo-8-fluoro-2H-1,4-benzoxazin-3(4H)-one

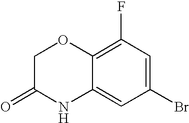

To a suspension of 4-bromo-2-fluoro-6-nitrophenol (20.0 g, 84.7 mmol) and K₂CO₃ (12.9 g, 93.2 mmol) in DMSO (150 ml) was added ethyl bromoacetate (10.4 ml, 93.2 mmol) at room temperature. After stirring 1 hr at 80° C., the reaction mixture was treated with EtOAc and H₂O. The organic layer was separated, washed successively with H₂O and brine, dried over Na₂SO₄ and concentrated in vacuo. The residue was dissolved in AcOH (150 ml). Fe (14.2 g, 254 mmol) was added to the resultant solution at room temperature. After stirring for 3 hr at 90° C., the reaction mixture was filtrated, and the filtrate was concentrated in vacuo. The residue was treated with THF, EtOAc and brine. The organic layer was separated, washed with brine and concentrated in vacuo. The residue was recrystallized from THF, EtOAc and hexane to give the title compound (13.24 g, 64%).

¹H-NMR (300 MHz, DMSO-d₆) δ: 4.68 (2H, s), 6.87 (1H, t, J=2.0 Hz), 7.21 (1H, dd, J=10.0, 2.0 Hz), 10.99 (1H, s).

### Preparation 46

### 8-Fluoro-6-(4,4,5,5-tetramethyl-1,3,2-dioxaborolan-2-yl)-2H-1,4-benzoxazin-3(4H)-one

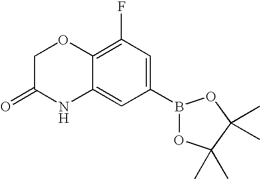

A mixture of 6-bromo-8-fluoro-2H-1,4-benzoxazin-3(4H)-one (8.00 g, 32.5 mmol), bis(pinacolato)diboron (9.08 g, 35.8 mmol), [1,1-bis(diphenylphosphino)ferrocene]dichloropalladium(II) dichloromethane adduct (1.33 g, 1.63 mmol) and potassium acetate (11.2 g, 114 mmol) in degassed 1,4-dioxane (320 ml) was stirred at 90° C. for 13 hr under an argon atmosphere. The reaction mixture was treated with EtOAc and H₂O. The organic layer was separated, washed with brine, dried over Na₂SO₄ and concentrated in vacuo. The residue was purified by column chromatography and recrystallized from EtOAc and hexane to give the title compound (9.48 g, 99%).

¹H-NMR (300 MHz, DMSO-d₆) δ: 1.28 (12H, s), 4.70 (2H, s), 6.99-7.08 (2H, m), 10.90 (1H, s).

### Preparation 47

### 2-(8-Fluoro-3-oxo-3,4-dihydro-2H-1,4-benzoxazin-6-yl)-3-phenylacrylaldehyde

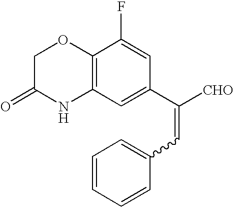

To a degassed mixture of THF (80 ml) and H₂O (16 ml) were added 8-fluoro-6-(4,4,5,5-tetramethyl-1,3,2-dioxaborolan-2-yl)-2H-1,4-benzoxazin-3(4H)-one (1.00 g, 3.41 mmol), α-bromocinnamaldehyde (865 mg, 4.09 mmol), [1,1-bis(diphenylphosphino)ferrocene]dichloropalladium(II) dichloromethane adduct (557 mg, 0.682 mmol) and Cs₂CO₃ (3.34 g, 10.2 mmol) at room temperature. After stirring under reflux for 13 hr under an argon atmosphere, the reaction mixture was treated with EtOAc and H₂O. The organic layer was separated, washed with brine, dried over Na₂SO₄ and concentrated in vacuo. The residue was purified by column chromatography to give the title compound (1.09 g, quant.).

¹H-NMR (300 MHz, DMSO-d₆) δ: 4.72 (2H, s), 6.49 (1H, t, J=1.5 Hz), 6.68 (1H, dd, J=11.0, 1.5 Hz), 7.26-7.40 (5H, m), 7.70 (1H, s), 9.70 (1H, s), 10.89 (1H, s).

### Example 113

### 6-(2-Amino-6-phenyl-6H-1,3-thiazin-5-yl)-8-fluoro-2H-1,4-benzoxazin-3(4H)-one

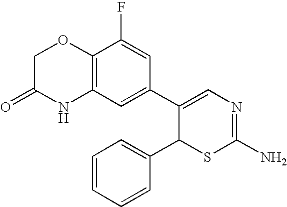

A solution of thiourea (311 mg, 4.09 mmol) and 2-(8-fluoro-3-oxo-3,4-dihydro-2H-1,4-benzoxazin-6-yl)-3-phenylacrylaldehyde (1.01 g, 3.41 mmol) in a mixed solvent of conc. HCl (4.0 ml), H₂O (8.0 ml) and 1,4-dioxane (40 ml) was stirred for 12 hr under reflux. The reaction mixture was treated with EtOAc and 1N NaOH. The organic layer was separated, washed with brine, dried over Na₂SO₄ and concentrated in vacuo. The residue was purified by column chromatography and recrystallized from EtOAc and hexane to give the title compound (1.02 g, 89%).

¹H-NMR (300 MHz, DMSO-d₆) δ: 4.59 (2H, s), 5.21 (1H, s), 6.68-6.72 (1H, m), 6.89 (1H, dd, J=12.5, 2.0 Hz), 6.96 (2H, s), 7.18-7.34 (6H, m), 10.80 (1H, s).

### Example 114

### 8-Fluoro-6-(7-phenyl-7H-imidazo[2,1-b][1,3]thiazin-6-yl)-2H-1,4-benzoxazin-3(4H)-one

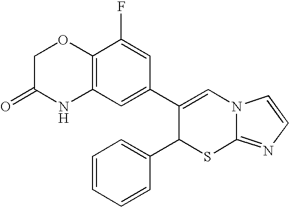

A solution of chloroacetaldehyde (45% aqueous solution, 4.22 g, 24.2 mmol) and 6-(2-amino-6-phenyl-6H-1,3-thiazin-5-yl)-8-fluoro-2H-1,4-benzoxazin-3(4H)-one (1.02 g, 3.03 mmol) in a mixed solvent of EtOH (15 ml) and 1,2-dimethoxyethane (15 ml) was stirred for 12 hr under reflux. The reaction mixture was treated with EtOAc and 1N NaOH. The organic layer was separated, washed with brine, dried over Na₂SO₄ and concentrated in vacuo. The residue was purified by column chromatography and recrystallized from THF and hexane to give the title compound (224 mg, 20%).

¹H-NMR (300 MHz, DMSO-d₆) δ: 4.65 (2H, s), 5.56 (1H, s), 6.69-6.78 (1H, m), 6.96 (1H, d, J=1.5 Hz), 7.10 (1H, dd, J=12.0, 2.0 Hz), 7.17-7.35 (5H, m), 7.57 (1H, d, J=1.5 Hz), 7.89 (1H, s), 10.96 (1H, s).

### Preparation 48

### 2-(8-Fluoro-3-oxo-3,4-dihydro-2H-1,4-benzoxazin-6-yl)-3-(4-fluorophenyl)acrylaldehyde

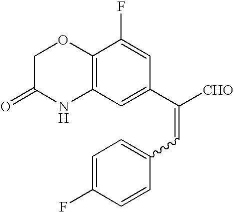

The title compound was obtained from 8-fluoro-6-(4,4,5,5-tetramethyl-1,3,2-dioxaborolan-2-yl)-2H-1,4-benzoxazin-3(4H)-one and 3-(4-fluorophenyl)-2-iodoacrylaldehyde according to a method similar to the procedure for 2-(8-fluoro-3-oxo-3,4-dihydro-2H-1,4-benzoxazin-6-yl)-3-phenylacrylaldehyde (Preparation 47).

¹H-NMR (300 MHz, DMSO-d₆) δ: 4.72 (2H, s), 6.37-6.54 (1H, m), 6.69 (1H, dd, J=11.0, 2.0 Hz), 7.16-7.28 (2H, m), 7.29-7.41 (2H, m), 7.70 (1H, s), 9.68 (1H, s), 10.90 (1H, s).

### Example 115

### 6-[2-Amino-6-(4-fluorophenyl)-6H-1,3-thiazin-5-yl]-8-fluoro-2H-1,4-benzoxazin-3(4H)-one

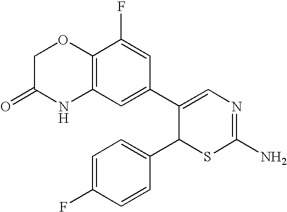

The title compound was obtained from 2-(8-fluoro-3-oxo-3,4-dihydro-2H-1,4-benzoxazin-6-yl)-3-(4-fluorophenyl)acrylaldehyde and thiourea according to a method similar to the procedure for 6-(2-amino-6-phenyl-6H-1,3-thiazin-5-yl)-8-fluoro-2H-1,4-benzoxazin-3(4H)-one (Example 113).

1H-NMR (300 MHz, DMSO-d₆) δ: 4.59 (2H, s), 5.24 (1H, s), 6.64-6.70 (1H, m), 6.89 (1H, dd, J=12.5, 2.0 Hz), 6.99 (2H, s), 7.08-7.17 (2H, m), 7.22-7.33 (3H, m), 10.80 (1H, s).

### Example 116

### 8-Fluoro-6-[7-(4-fluorophenyl)-7H-imidazo[2,1-b][1,3]thiazin-6-yl]-2H-1,4-benzoxazin-3(4H)-one

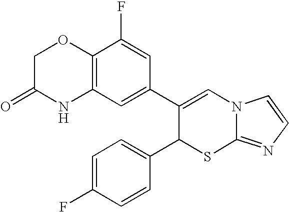

The title compound was obtained from 6-[2-amino-6-(4-fluorophenyl)-6H-1,3-thiazin-5-yl]-8-fluoro-2H-1,4-benzoxazin-3(4H)-one and chloroacetaldehyde according to a method similar to the procedure for 8-fluoro-6-(7-phenyl-7H-imidazo[2,1-b][1,3]thiazin-6-yl)-2H-1,4-benzoxazin-3(4H)-one (Example 114).

¹H-NMR (300 MHz, DMSO-d₆) δ: 4.66 (2H, s), 5.73 (1H, s), 6.67-6.77 (1H, m), 7.08-7.34 (6H, m), 7.71 (1H, d, J=1.5 Hz), 7.98 (1H, s), 11.00 (1H, s).

### Preparation 49

### 4-Bromo-2-chloro-6-nitrophenol

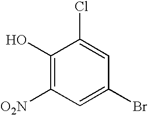

To a solution of 4-bromo-2-chlorophenol (25.0 g, 120 mmol) in propionic acid (160 ml) were added 70% nitric acid (0.8 ml, 12.0 mmol), sulfuric acid (1.6 ml, 30 mmol) and an aqueous sodium nitrite solution (3.3 mg, 0.048 mmol in a 5 drops of water) at 30° C. Additional 70% nitric acid (64 ml, 100 mmol) was added to the mixture over 10 min. After stirring for 3 hr at 30° C., the reaction mixture was diluted with H₂O. The orange precipitate was collected by filtration, washed with H₂O and dried in vacuo to give the title compound (26.7 g, 88%).

¹H-NMR (300 MHz, DMSO-d₆) δ: 8.07 (1H, d, J=2.5 Hz), 8.10 (1H, d, J=2.5 Hz).

### Preparation 50

### 2-Amino-4-bromo-6-chlorophenol

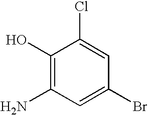

A suspension of 4-bromo-2-chloro-6-nitrophenol (2.15 g, 8.51 mmol), Fe (2.38 g, 42.6 mmol) and CaCl₂ (94 mg, 0.85 mmol) in 80% aqueous EtOH (100 ml) was stirred for 2 hr at 80° C. After filtration of the reaction mixture, the filtrate was concentrated in vacuo. The residue was treated with EtOAc and H₂O. The organic layer was separated, washed with H₂O and brine, dried over Na₂SO₄ and concentrated in vacuo. The residue was purified by column chromatography to give the title compound (730 mg, 39%).

¹H-NMR (300 MHz, DMSO-d₆) δ: 5.23 (2H, brs), 6.66 (1H, d, J=2.5 Hz), 6.71 (1H, d, J=2.5 Hz), 9.05 (1H, brs).

### Preparation 51

### 6-Bromo-8-chloro-2H-1,4-benzoxazin-3(4H)-one

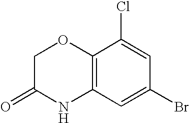

To a solution of 2-amino-4-bromo-6-chlorophenol (730 mg, 3.28 mmol) and Na₂CO₃ (470 mg, 4.43 mmol) in a mixed solvent of isobutyl methyl ketone (30 ml) and H₂O (30 ml) was added chloroacetyl chloride (500 mg, 4.43 mmol) at 0° C. After stirring vigorously for 5 hr under reflux, the reaction mixture was extracted with EtOAc-THF. The organic layer was washed successively with H₂O and brine, dried over Na₂SO₄ and concentrated in vacuo. The residue was recrystallized from EtOAc-THF-hexane to give the title compound (715 mg, 83%).

¹H NMR (300 MHz, DMSO-d₆) δ: 4.72 (2H, s), 6.99 (1H, d, j=2.5 Hz), 7.30 (1H, d, J=2.5 Hz), 10.98 (1H, brs).

### Preparation 52

### 8-Chloro-6-(4,4,5,5-tetramethyl-1,3,2-dioxaborolan-2-yl)-2H-1,4-benzoxazin-3(4H)-one

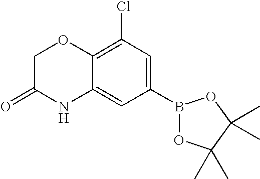

A mixture of 6-bromo-8-chloro-2H-1,4-benzoxazin-3(4H)-one (715 mg), bis(pinacolato)diboron (760 mg), [1,1-bis(diphenylphosphino)ferrocene]dichloropalladium(II) dichloromethane adduct (110 mg) and potassium acetate (934 mg) in degassed 1,4-dioxane (60 ml) was stirred at 90° C. for 12 hr under an argon atmosphere. The reaction mixture was treated with EtOAc and H₂O. The organic layer was separated, washed with brine, dried over Na₂SO₄ and concentrated in vacuo. The residue was purified by silica gel column chromatography using hexane/EtOAc as an eluent to give the title compound (841 mg).

¹H-NMR (300 MHz, DMSO-d₆) δ: 1.28 (12H, s), 4.74 (2H, s), 7.14 (1H, d, J=1.5 Hz), 7.23 (1H, d, J=1.5 Hz), 10.89 (1H, s).

### Preparation 53

### 2-(8-Chloro-3-oxo-3,4-dihydro-2H-1,4-benzoxazin-6-yl)-3-(4-fluorophenyl)acrylaldehyde

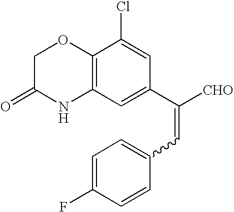

To a degassed mixture of THF (65 ml) and H₂O (13 ml) were added 8-chloro-6-(4,4,5,5-tetramethyl-1,3,2-dioxaborolan-2-yl)-2H-1,4-benzoxazin-3(4H)-one (841 mg), α-bromocinnamaldehyde (900 mg), [1,1-bis(diphenylphosphino)ferrocene]dichloropalladium(II) dichloromethane adduct (444 mg) and Cs2CO3 (2.65 g) at r.t. After stirring under reflux for 12 hr under an argon atmosphere, the reaction mixture was treated with EtOAc and H₂O. The organic layer was separated, washed with brine, dried over Na₂SO₄ and concentrated in vacuo. The residue was purified by silica gel column chromatography using hexane/EtOAc as an eluent to give the title compound (870 mg).

MS (ESI) 332 (M+H).

¹H-NMR (300 MHz, DMSO-d₆) δ: 4.76 (2H, s), 6.61 (1H, d, J=2.0 Hz), 6.84 (1H, d, J=2.0 Hz), 7.22 (2H, t, J=9.0 Hz), 7.36 (2H, dd, J=9.0, 6.0 Hz), 7.71 (1H, s), 9.68 (1H, s), 10.88 (1H, brs).

### Example 117

### 6-[2-Amino-6-(4-fluorophenyl)-6H-1,3-thiazin-5-yl]-8-chloro-2H-1,4-benzoxazin-3(4H)-one

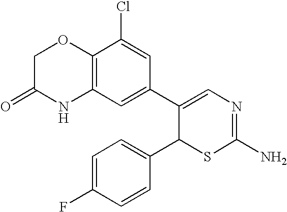

A solution of thiourea (240 mg) and 2-(8-chloro-3-oxo-3,4-dihydro-2H-1,4-benzoxazin-6-yl)-3-(4-fluorophenyl)acrylaldehyde (870 mg) in a mixture of conc. HCl (2 ml), H₂O (4 ml) and 1,4-dioxane (20 ml) was stirred for 12 hr under reflux. The reaction mixture was treated with EtOAc and 1N NaOH. The organic layer was separated, washed with brine, dried over Na₂SO₄ and concentrated in vacuo. The residue was purified by silica gel column chromatography using hexane/EtOAc as an eluent and recrystallized from EtOAc-THF-hexane to give the title compound (540 mg).

¹H-NMR (300 MHz, DMSO-d₆) δ: 4.63 (2H, s), 5.27 (1H, s), 6.82 (1H, d, J=2.0 Hz), 6.98 (2H, brs), 7.01 (1H, d, J=2.0 Hz), 7.13 (2H, t, J=9.0 Hz), 7.23 (1H, s), 7.28 (2H, dd, J=9.0, 5.5 Hz), 10.79 (1H, brs).

### Example 118

### 8-Chloro-6-[7-(4-fluorophenyl)-7H-imidazo[2,1-b][1,3]thiazin-6-yl]-2H-1,4-benzoxazin-3(4H)-one

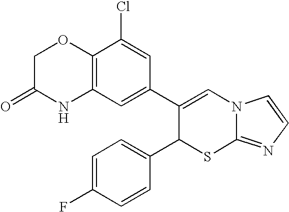

A solution of chloroacetaldehyde (45% aqueous solution, 1.92 g) and 6-[2-amino-6-(4-fluorophenyl)-6H-1,3-thiazin-5-yl]-8-chloro-2H-1,4-benzoxazin-3(4H)-one (540 mg) in a mixture of EtOH (10 ml) and 1,2-dimethoxyethane (10 ml) was stirred for 13 hr under reflux. The reaction mixture was treated with EtOAc and 1N NaOH. The organic layer was separated, washed with brine, dried over Na₂SO₄ and concentrated in vacuo. The residue was purified by silica gel column chromatography using hexane/EtOAc as an eluent and recrystallized from THF and hexane to give the title compound (104 mg).

¹H-NMR (300 MHz, DMSO-d₆) δ: 4.69 (2H, s), 5.60 (1H, s), 6.83 (1H, d, J=2.5 Hz), 6.97 (1H, d, J=1.5 Hz), 7.14 (2H, t, J=9.0 Hz), 7.20-7.29 (3H, m), 7.56 (1H, d, J=1.5 Hz), 7.90 (1H, s), 10.94 (1H, brs).

### Example 119

### 6-{7-[(2-Hydroxyethyl)amino]-2-phenyl-2H-chromen-3-yl}-2H-1,4-benzoxazin-3(4H)-one

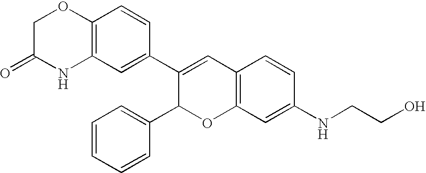

A mixture of 6-(7-iodo-2-phenyl-2H-chromen-3-yl)-2H-1,4-benzoxazin-3(4H)-one (96 mg), ethanolamine (37 mg), copper(I) iodide (19 mg), L-proline (12 mg) and potassium carbonate (111 mg) in DMSO (1.8 mL) was heated at 90° C. for 20 hr, cooled, and treated with ethyl acetate and saturated ammonium chloride solution. The organic layer was separated, washed with water, dried over MgSO₄ and concentrated. The residue was chromatographed on silica gel using n-hexane/ethyl acetate as an eluent to give the title compound as a foam (40 mg).

¹H-NMR (300 MHz, CDCl₃) δ: 2.30-2.50 (br, 1H), 3.22 (t, J=5.1 Hz, 2H), 3.77 (t, J=5.1 Hz, 2H), 4.10-4.30 (br, 1H), 4.54 (s, 2H), 6.07-6.21 (m, 3H), 6.66 (d, J=1.8 Hz, 1H), 6.80-6.97 (m, 4H), 7.20-7.25 (m, 3H), 7.36-7.40 (m, 2H), 8.72 (s, 1H).

### Example 120

### 6-[7-(2-Hydroxyethoxy)-2-phenyl-2H-chromen-3-yl]-2H-1,4-benzoxazin-3(4H)-one

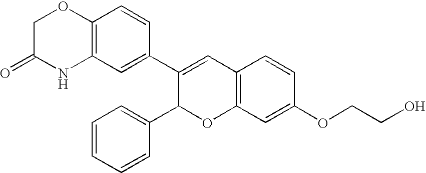

A mixture of 6-(7-iodo-2-phenyl-2H-chromen-3-yl)-2H-1,4-benzoxazin-3(4H)-one (75 mg), copper(I) iodide (9 mg), 1,10-phenanthroline (17 mg), cesium carbonate (104 mg) and ethylene glycol (0.9 mL) was heated at 110° C. for 40 hr, cooled, and treated with ethyl acetate and water. The organic layer was separated, washed with water, dried over MgSO₄ and concentrated. The residue was chromatographed on silica gel using n-hexane/ethyl acetate as an eluent to give the title compound as a foam (7 mg).

¹H-NMR (300 MHz, CDCl₃) δ: 2.00-2.20 (br, 1H), 3.83-4.01 (m, 4H), 4.59 (s, 2H), 6.15 (s, 1H), 6.35 (d, J=2.1 Hz, 1H), 6.45 (dd, J=8.1, 2.4 Hz, 1H), 6.78 (d, J=2.1 Hz, 1H), 6.88 (d, J=8.4 Hz, 1H), 6.96-7.05 (m, 3H), 7.22-7.30 (m, 3H), 7.38-7.42 (m, 2H), 8.33 (br, 1H).

### Example 121

### 6-[7-(Ethylamino)-2-phenyl-2H-chromen-3-yl]-2H-1,4-benzoxazin-3(4H)-one

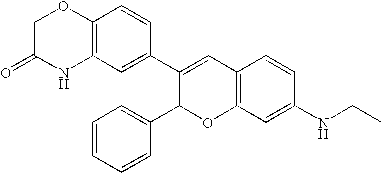

According to the method of Example 119, 6-(7-iodo-2-phenyl-2H-chromen-3-yl)-2H-1,4-benzoxazin-3(4H)-one (75 mg) and 70% ethylamine (0.5 mL) were reacted to give the title compound as a foam (20 mg).

¹H-NMR (300 MHz, CDCl₃) δ: 1.19 (t, J=7.2 Hz, 3H), 3.08 (q, J=7.2 Hz, 2H), 3.65-3.80 (br, 1H), 4.58 (s, 2H), 6.02 (d, J=2.1 Hz, 1H), 6.11-6.14 (m, 2H), 6.78 (d, J=2.1 Hz, 1H), 6.86 (d, J=8.4 Hz, 1H), 6.91-6.96 (m, 3H), 7.24-7.29 (m, 3H), 7.40-7.43 (m, 2H), 8.50 (s, 1H).

### Example 122

### 6-[7-(Methylsulfonyl)-2-phenyl-2H-chromen-3-yl]-2H-1,4-benzoxazin-3(4H)-one

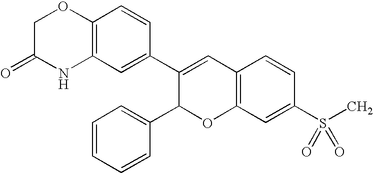

A mixture of 6-(7-iodo-2-phenyl-2H-chromen-3-yl)-2H-1,4-benzoxazin-3(4H)-one (76 mg), sodium methanesulfinate (40 mg), copper(I) iodide (15 mg), L-proline (18 mg) and powdered NaOH (6 mg) in DMSO (1 mL) was heated at 90° C. for 14 hr, cooled, and treated with ethyl acetate and water. The organic layer was separated, washed with water, dried over MgSO₄ and concentrated. The residue was chromatographed on silica gel using n-hexane/ethyl acetate as an eluent to give the title compound as colorless crystals (54 mg).

¹H-NMR (300 MHz, DMSO-d₆) δ: 3.18 (s, 3H), 4.58 (s, 2H), 6.57 (s, 1H), 6.95 (d, J=8.4 Hz, 1H), 7.08 (d, J=2.4 Hz, 1H), 7.14-7.45 (m, 9H), 7.53 (d, J=8.4 Hz, 1H), 10.74 (s, 1H).

### Example 123

### 6-{7-[(2-Methoxyethyl)amino]-2-phenyl-2H-chromen-3-yl}-2H-1,4-benzoxazin-3(4H)-one

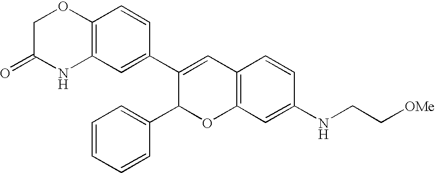

According to the method of Example 119, 6-(7-iodo-2-phenyl-2H-chromen-3-yl)-2H-1,4-benzoxazin-3(4H)-one (75 mg) and 2-methoxyethylamine (0.1 mL) were reacted to give the title compound as colorless crystals (44 mg).

mp. 185-192° C. (ethyl acetate).

¹H-NMR (300 MHz, CDCl₃) δ: 3.21 (t, J=5.1 Hz, 2H), 3.34 (s, 3H), 3.54 (t, J=5.1 Hz, 2H), 4.10-4.20 (br, 1H), 4.56 (s, 2H), 6.04 (d, J=2.4 Hz, 1H), 6.12 (s, 1H), 6.15 (dd, J=8.4, 2.4 Hz, 1H), 6.79 (d, J=1.8 Hz, 1H), 6.84 (d, J=8.4 Hz, 1H), 6.91-6.94 (m, 3H), 7.22-7.27 (m, 3H), 7.40-7.43 (m, 2H), 8.92 (s, 1H).

### Example 124

### 6-{7-[(3-Hydroxypropyl)amino]-2-phenyl-2H-chromen-3-yl}-2H-1,4-benzoxazin-3(4H)-one

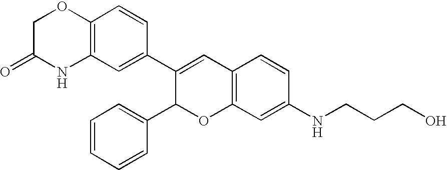

According to the method of Example 119, 6-(7-iodo-2-phenyl-2H-chromen-3-yl)-2H-1,4-benzoxazin-3(4H)-one (75 mg) and 3-amino-1-propanol (0.15 mL) were reacted to give the title compound as crystals (36 mg).

mp. 215-217° C. (ethyl acetate).

¹H-NMR (300 MHz, CDCl₃) δ: 1.60-1.70 (br, 1H), 1.85 (quintet, J=6.0 Hz, 2H), 3.21 (t, J=6.0 Hz, 2H), 3.76 (t, J=6.0 Hz, 2H), 4.57 (s, 2H), 6.06 (d, J=1.8 Hz, 1H), 6.12 (s, 1H), 6.16 (dd, J=8.4, 2.4 Hz, 1H), 6.73 (d, J=1.8 Hz, 1H), 6.83-6.95 (m, 4H), 7.23-7.27 (m, 3H), 7.39-7.42 (m, 2H), 8.35-8.50 (br, 1H).

### Example 125

### 6-(7-{[2-(Dimethylamino)ethyl]amino}-2-phenyl-2H-chromen-3-yl)-2H-1,4-benzoxazin-3(4H)-one

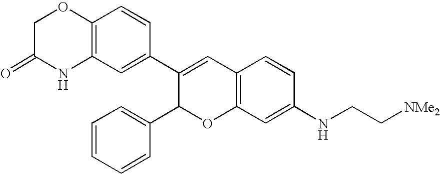

According to the method of Example 119, 6-(7-iodo-2-phenyl-2H-chromen-3-yl)-2H-1,4-benzoxazin-3(4H)-one (75 mg) and N,N-dimethylethane-1,2-diamine (0.15 mL) were reacted to give the title compound as crystals (22 mg).

¹H-NMR (300 MHz, CDCl₃) δ: 2.20 (s, 6H), 2.49 (t, J=6.0 Hz, 2H), 3.07 (t, J=6.0 Hz, 2H), 4.30-4.50 (br, 1H), 4.57 (s, 2H), 6.03 (d, J=1.8 Hz, 1H), 6.11 (s, 1H), 6.15 (dd, J=8.4, 2.4 Hz, 1H), 6.76 (d, J=2.4 Hz, 1H), 6.85 (d, J=8.4 Hz, 1H), 6.91-6.96 (m, 3H), 7.22-7.29 (m, 3H), 7.40-7.43 (m, 2H), 8.74 (brs, 1H).

### Preparation 54

### (2-Mercaptopyridin-3-yl)methanol

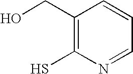

To a suspension of 2-mercaptonicotinic acid (2.00 g) in THF (80 mL) was added 1M borane tetrahydrofuran complex in THF (30 mL), and the mixture was stirred at room temperature for 3 hr and at 50° C. for 2 hr. The mixture was cooled with an ice-bath, quenched by the addition of 1N HCl (100 mL), neutralized with 8N NaOH, salted out by the addition of NaCl, and extracted with THF/ethyl acetate (1/1, 2×). The organic layers were combined, dried over MgSO₄ and concentrated. The residue was suspended in ethyl acetate/THF and collected by filtration to give the title compound as crystals (1.15 g).

¹H-NMR (300 MHz, DMSO-d₆) δ: 4.41 (s, 2H), 5.30 (br, 1H), 6.54 (br, 1H), 6.86 (t, J=6.6 Hz, 1H), 7.59-7.66 (m, 2H).

### Preparation 55

### 6-[{[3-(Hydroxymethyl)pyridin-2-yl]thio}(phenyl)acetyl]-2H-1,4-benzoxazin-3(4H)-one

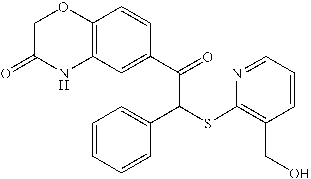

To a mixture of 6-[bromo(phenyl)acetyl]-2H-1,4-benzoxazin-3(4H)-one (0.52 g) and (2-mercaptopyridin-3-yl)methanol (0.42 g) in DMF (10 mL) was added triethylamine (0.84 mL). The mixture was stirred at room temperature for 16 hr, poured into water and extracted with ethyl acetate. The extract was washed with saturated aqueous NaHCO₃, dried over MgSO₄ and concentrated. The residue was chromatographed on silica gel using n-hexane/ethyl acetate as an eluent to give the title compound as a colorless foam (0.47 g).

¹H-NMR (300 MHz, CDCl₃) δ: 3.97 (br, 1H), 4.52 (d, J=13.8 Hz, 1H), 4.55 (s, 2H), 4.66 (d, J=13.8 Hz, 1H), 6.68 (s, 1H), 6.79 (dd, J=7.5, 4.8 Hz, 1H), 6.87 (d, J=9.0 Hz, 1H), 7.17-7.26 (m, 3H), 7.40-7.43 (m, 2H), 7.51 (d, J=7.5 Hz, 1H), 7.69-7.71 (m, 2H), 7.95 (dd, J=7.8, 4.8 Hz, 1H), 9.61 (s, 1H).

### Preparation 56

### 6-[{[3-(Bromomethyl)pyridin-2-yl]thio}(phenyl)acetyl]-2H-1,4-benzoxazin-3(4H)-one

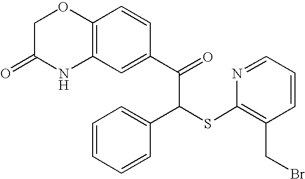

To a mixture of 6-[{[3-(hydroxymethyl)pyridin-2-yl]thio}(phenyl)acetyl]-2H-1,4-benzoxazin-3(4H)-one (0.38 g) and triphenylphosphine (0.38 g) in acetonitrile (6 mL) was added N-bromosuccinimide (0.25 g) under ice-cooling. The mixture was stirred at 0° C. for 1 hr and treated with ethyl acetate and water. The organic layer was separated, washed with 10% Na₂S₂O₃ solution and saturated aqueous NaHCO₃, dried over MgSO₄ and concentrated. The residue was chromatographed on silica gel using n-hexane/ethyl acetate as an eluent to give the title compound as a colorless foam (0.32 g).

¹H-NMR (300 MHz, CDCl₃) δ: 4.45 (d, J=11.1 Hz, 1H), 4.53 (d, J=11.1 Hz, 1H), 4.66 (s, 2H), 6.73 (s, 1H), 6.94-7.00 (m, 2H), 7.25-7.36 (m, 3H), 7.49-7.55 (m, 4H), 7.76 (dd, J=8.4, 2.4 Hz, 1H), 8.12-8.15 (m, 2H).

### Preparation 57

### [(2-{[2-oxo-2-(3-oxo-3,4-dihydro-2H-1,4-benzoxazin-6-yl)-1-phenylethyl]thio}pyridin-3-yl)methyl](triphenyl)phosphonium Bromide

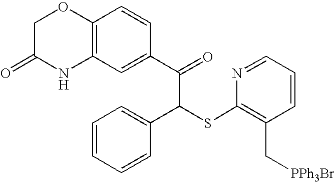

A mixture of 6-[{[3-(bromomethyl)pyridin-2-yl]thio}(phenyl)acetyl]-2H-1,4-benzoxazin-3(4H)-one (0.32 g) and triphenylphosphine (0.18 g) in toluene/acetonitrile (2/1, 6 mL) was refluxed for 2 hr and concentrated. The residual crystals were suspended in toluene and collected by filtration to give the title compound (0.40 g).

¹H-NMR (300 MHz, CDCl₃+DMSO-d₆) δ: 4.59 (s, 2H), 4.91-5.15 (m, 2H), 6.46 (s, 1H), 6.87-6.92 (m, 2H), 7.14-7.85 (m, 23H), 8.22-8.25 (m, 1H), 10.69 (s, 1H).

### Example 126

### 6-(2-Phenyl-2H-thiopyrano[2,3-b]pyridin-3-yl)-2H-1,4-benzoxazin-3(4H)-one

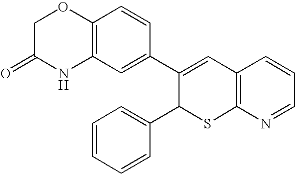

To a suspension of [(2-{[2-oxo-2-(3-oxo-3,4-dihydro-2H-1,4-benzoxazin-6-yl)-1-phenylethyl]thio}pyridin-3-yl)methyl](triphenyl)phosphonium bromide (0.40 g) in toluene (6 mL) was added 2.5 M sodium methoxide solution in methanol (0.4 mL). The mixture was heated at 90° C. for 0.5 hr, cooled, diluted with brine and extracted with THF/ethyl acetate (1/2). The extract was dried over MgSO₄ and concentrated, and the residue was chromatographed on silica gel using ethyl acetate/n-hexane as an eluent to give the title compound as colorless crystals (0.18 g).

mp. 242-244° C. (ethyl acetate).

¹H-NMR (300 MHz, CDCl₃) δ: 4.57 (s, 2H), 5.06 (s, 1H), 6.86 (d, J=8.4 Hz, 1H), 6.95-7.26 (m, 9H), 7.45 (dd, J=7.5, 1.5 Hz, 1H), 8.23 (dd, J=4.8, 1.5 Hz, 1H), 9.83 (s, 1H).

### Preparation 58

### 2-Hydroxy-4-iodobenzoic Acid

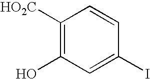

A suspension of 4-aminosalicylic acid (60.6 g), water (240 mL), c-H₂SO₄ (90 mL) and acetic acid (240 mL) was cooled with an ice-bath. A solution of sodium nitrite (30.0 g) in water (60 mL) was added dropwise to the suspension over 30 min and the mixture was stirred at 0° C. for 1 hr. Then a solution of potassium iodide (200 g) in water (160 mL) was added dropwise over 30 min and the cooling-bath was removed. The mixture was stirred at room temperature for 20 hr, diluted with water and extracted with ethyl acetate (three times). The extracts were combined, washed with 5% Na₂S₂O₃ solution and brine, dried over MgSO₄ and concentrated. The residue was suspended in acetonitrile and collected by filtration to give the title compound as a powder (35.0 g).

¹H-NMR (300 MHz, DMSO-d₆) δ: 7.30 (dd, J=8.1, 1.8 Hz, 1H), 7.38 (d, J=1.8 Hz, 1H), 7.51 (d, J=8.1 Hz, 1H).

### Preparation 59

### Methyl 2-Hydroxy-4-iodobenzoate

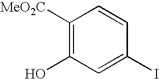

To a solution of 2-hydroxy-4-iodobenzoic acid (35.0 g) in methanol (700 mL) was added dropwise thionyl chloride (40 mL). The mixture was refluxed for 14 hr and concentrated. The residue was chromatographed on silica gel using n-hexane/ethyl acetate as an eluent to give the title compound as crystals (34.2 g).

mp. 69° C. (ethyl acetate/n-hexane).

¹H-NMR (300 MHz, CDCl₃) δ: 3.94 (s, 3H), 7.23 (dd, J=8.4, 1.8 Hz, 1H), 7.40 (d, J=1.8 Hz, 1H), 7.50 (d, J=8.4 Hz, 1H), 10.74 (s, 1H).

### Preparation 60

### Methyl 2-{[(Dimethylamino)carbonothioyl]oxy}-4-iodobenzoate

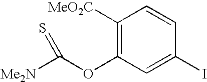

To a mixture of methyl 2-hydroxy-4-iodobenzoate (11.00 g) and 1,8-diazabicyclo[5,4,0]undec-7-ene (12.2 g) in DMF (50 mL) was added N,N-dimethylthiocarbamoyl chloride (7.42 g). The mixture was stirred at room temperature for 14 hr and at 60° C. for 3 hr, poured into water and extracted with ethyl acetate. The extract was washed with water, dried over MgSO₄ and concentrated. The residue was chromatographed on silica gel using ethyl acetate as an eluent, and the product was recrystallized from ethyl acetate/diisopropyl ether to give the title compound as crystals (8.80 g).

¹H-NMR (300 MHz, CDCl₃) δ: 3.38 (s, 3H), 3.46 (s, 3H), 3.83 (s, 3H), 7.50 (d, J=0.9 Hz, 1H), 7.64-7.71 (m, 2H).

### Preparation 61

### Methyl 2-{[(Dimethylamino)carbonyl]thio}-4-iodobenzoate

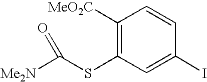

Methyl 2-{[(dimethylamino)carbonothioyl]oxy}-4-iodobenzoate (8.80 g) was heated at 190° C. for 3 hr. Column chromatography on silica gel using n-hexane/ethyl acetate as an eluent gave the title compound as an oil (4.37 g).

¹H-NMR (300 MHz, CDCl₃) δ: 3.00-3.20 (m, 6H), 3.87 (s, 3H), 7.60 (d, J=8.4 Hz, 1H), 7.76 (dd, J=8.4, 1.8 Hz, 1H), 7.97 (d, J=1.8 Hz, 1H).

### Preparation 62

### (4-Iodo-2-mercaptobenzyl)(triphenyl)phosphonium Bromide

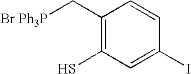

To a solution of methyl 2-{[(dimethylamino)carbonyl]thio}-4-iodobenzoate (5.20 g) in methanol (120 mL) was added sodium methoxide (2.32 g). The mixture was refluxed for 1 hr under a nitrogen atmosphere and concentrated. The residue was treated with 1N HCl and extracted with ethyl acetate. The extract was washed with brine, dried over MgSO₄ and concentrated to give crude methyl 4-iodo-2-mercaptobenzoate (4.44 g). To a cooled (0° C.) suspension of LiAlH₄ (0.67 g) in THF (150 mL) was added a solution of methyl 4-iodo-2-mercaptobenzoate (4.44 g) in THF (20 mL). The mixture was stirred at 0° C. for 1 hr, quenched by the addition of water, diluted with 1N HCl and extracted with ethyl acetate (3×). The extracts were combined, dried over MgSO₄ and concentrated to give crude (4-iodo-2-mercaptophenyl)methanol (3.70 g).

A mixture of (4-iodo-2-mercaptophenyl)methanol (3.70 g) and triphenylphosphine hydrobromide (4.74 g) in acetonitrile (50 mL) was refluxed for 3 hr and concentrated. The residue was suspended in ethyl acetate/acetonitrile and collected by filtration to give the title compound as colorless crystals (6.10 g).

¹H-NMR (300 MHz, DMSO-d₆) δ: 4.26 (s, 1H), 5.32 (d, J=14.1 Hz, 2H), 6.95 (dd, J=8.7, 2.4 Hz, 1H), 7.30-7.90 (m, 17H).

### Example 127

### 6-(7-Iodo-2-phenyl-2H-thiochromen-3-yl)-2H-1,4-benzoxazin-3(4H)-one

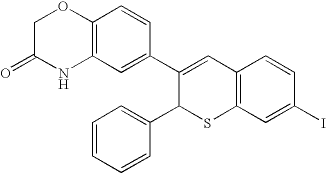

To a suspension of (4-iodo-2-mercaptobenzyl)(triphenyl)phosphonium bromide (4.28 g) in toluene (42 mL) was added 2.5 M sodium methoxide solution in methanol (2.9 mL) and the mixture was stirred at room temperature for 20 min. Then 6-[bromo(phenyl)acetyl]-2H-1,4-benzoxazin-3(4H)-one (2.51 g) was added and the mixture was refluxed for 0.5 hr. An 2.5 M sodium methoxide solution in methanol (2.9 mL) was added and the whole mixture was refluxed for an additional 4 hr, cooled and treated with ethyl acetate and water. The organic layer was separated, dried over MgSO₄ and concentrated. The residue was chromatographed on silica gel using ethyl acetate/n-hexane as an eluent and followed by recrystallization from ethyl acetate to give the title compound as colorless crystals (2.27 g).

mp. 208-212° C. (AcOEt).

¹H-NMR (300 MHz, DMSO-d₆) δ: 4.56 (s, 2H), 5.34 (s, 1H), 6.92 (d, J=8.1 Hz, 1H), 7.04 (s, 1H), 7.11 (d, J=8.1 Hz, 1H), 7.13-7.25 (m, 7H), 7.49-7.52 (m, 2H), 10.73 (s, 1H).

### Example 128

### 6-{7-[(2-Hydroxyethyl)amino]-2-phenyl-2H-thiochromen-3-yl}-2H-1,4-benzoxazin-3(4H)-one

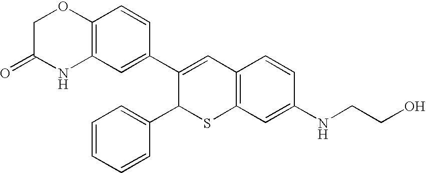

According to the method of Example 119, 6-(7-iodo-2-phenyl-2H-thiochromen-3-yl)-2H-1,4-benzoxazin-3(4H)-one (0.80 g) and 2-aminoethanol (0.85 g) were reacted to give the title compound as colorless crystals (0.62 g).

mp. 201-202° C. (ethyl acetate/diisopropyl ether).

¹H-NMR (300 MHz, DMSO-d₆) δ: 3.05 (q, J=6.0 Hz, 2H), 3.49 (q, J=6.0 Hz, 2H), 4.53 (s, 2H), 4.66 (t, J=6.0 Hz, 1H), 5.13 (s, 1H), 5.89 (t, J=6.0 Hz, 1H), 6.30 (d, J=1.8 Hz, 1H), 6.39 (dd, J=8.1, 2.1 Hz, 1H), 6.86 (d, J=9.3 Hz, 1H), 6.99-7.27 (m, 9H), 10.65 (s, 1H).

### Example 129

### 6-{(7-[(2-Methoxyethyl)amino]-2-phenyl-2H-thiochromen-3-yl}-2H-1,4-benzoxazin-3(4H)-one

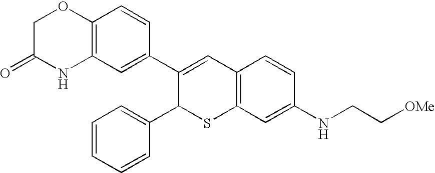

According to the method of Example 119, 6-(7-iodo-2-phenyl-2H-thiochromen-3-yl)-2H-1,4-benzoxazin-3(4H)-one (100 mg) and 2-methoxyethylamine (0.10 mL) were reacted to give the title compound as a foam (34 mg).

¹H-NMR (300 MHz, CDCl₃) δ: 3.22 (t, J=5.1 Hz, 2H), 3.34 (s, 3H), 3.54 (t, J=5.1 Hz, 2H), 4.10-4.25 (br, 1H), 4.56 (s, 2H), 4.82 (s, 1H), 6.37-6.40 (m, 2H), 6.81-7.30 (m, 10H), 8.79 (s, 1H).

### Example 130

### 3-(3-Oxo-3,4-dihydro-2H-1,4-benzoxazin-6-yl)-2-phenyl-2H-thiochromene-7-carbonitrile

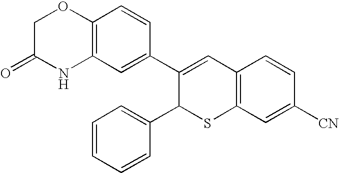

A mixture of 6-(7-iodo-2-phenyl-2H-thiochromen-3-yl)-2H-1,4-benzoxazin-3(4H)-one (100 mg), Zn(CN)₂ (35 mg) and Pd(PPh₃)₄ (23 mg) in DMF (1.8 mL) was heated at 85° C. for 14 hr under a nitrogen atmosphere. The mixture was treated with water and ethyl acetate, and the organic layer was separated, washed with water, dried and concentrated. The residue was chromatographed on silica gel using hexane/ethyl acetate as an eluent to give the title compound as a foam (13 mg).

¹H-NMR (300 MHz, CDCl₃) δ: 4.61 (s, 2H), 4.94 (s, 1H), 6.89-6.92 (m, 2H), 7.04 (dd, J=8.4, 2.1. Hz, 1H), 7.11 (s, 1H), 7.22-7.42 (m, 8H), 8.89 (brs, 1H).

### Preparation 63

### 2-(Hydroxymethyl)-4-methoxyphenol

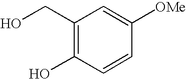

To a cooled (0° C.) solution of 5-methoxysalicylic acid (11.95 g) in THF (150 mL) was added 1M borane tetrahydrofuran complex in THF (200 mL), and the mixture was heated at 60° C. for 3 hr. The mixture was cooled with an ice-bath, quenched by the addition of 1N HCl (200 mL), stirred for 0.5 hr, salted out by the addition of NaCl, and extracted with ethyl acetate. The extract was dried over MgSO₄ and concentrated, and the residue was chromatographed on silica gel using n-hexane/ethyl acetate as an eluent to give the title compound as colorless crystals (3.00 g).

¹H-NMR (300 MHz, CDCl₃) δ: 2.10-2.30 (br, 1H), 3.75 (s, 3H), 4.83 (s, 2H), 6.62 (d, J=3.0 Hz, 1H), 6.77-6.81 (m, 3H).

### Preparation 64

### (2-Hydroxy-5-methoxybenzyl)(triphenyl)phosphonium Bromide

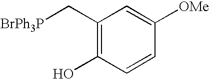

According to the method of Preparation 21, 2-(hydroxymethyl)-4-methoxyphenol (1.54 g) was reacted to give the title compound as colorless crystals (2.50 g).

¹H-NMR (300 MHz, CDCl₃+DMSO-d₆) δ: 3.48 (s, 3H), 4.67 (d, J=13.8 Hz, 2H), 6.32 (t, J=2.7 Hz, 1H), 6.69 (dt, J=9.0, 2.7 Hz, 1H), 6.77 (d, J=9.0 Hz, 1H), 7.52-7.88 (m, 15H), 8.94 (s, 1H).

### Example 131

### 6-(6-Methoxy-2-phenyl-2H-chromen-3-yl)-2H-1,4-benzoxazin-3(4H)-one

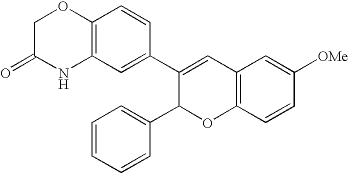

According to the method of Example 20, 6-[bromo(phenyl)acetyl]-2H-1,4-benzoxazin-3(4H)-one (0.69 g) and (2-hydroxy-5-methoxybenzyl)(triphenyl)phosphonium bromide (1.05 g) were reacted to give the title compound as colorless crystals (0.18 g).

mp. 200° C. (ethyl acetate).

¹H-NMR (300 MHz, DMSO-d₆) δ: 3.69 (s, 3H), 4.56 (s, 2H), 6.31 (s, 1H), 6.64 (s, 2H), 6.87 (s, 1H), 6.92 (d, J=8.4 Hz, 1H), 7.04 (d, J=2.1 Hz, 1H), 7.12-7.16 (m, 2H), 7.25-7.36 (m, 5H), 10.71 (s, 1H).

### Example 132

### Methyl 3-(3-Oxo-3,4-dihydro-2H-1,4-benzoxazin-6-yl)-2-phenyl-2H-thiochromene-7-carboxylate

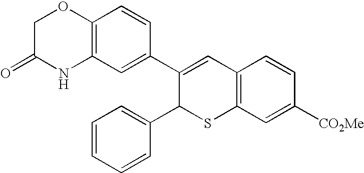

A mixture of 6-(7-iodo-2-phenyl-2H-thiochromen-3-yl)-2H-1,4-benzoxazin-3(4H)-one (700 mg), palladium(II) acetate (45 mg), 1,1′-bis(diphenylphosphino)ferrocene (110 mg), triethylamine (0.60 mL), methanol (1.6 mL) and DMF (5 mL) was heated at 75° C. for 20 hr under a carbon monoxide atmosphere. The mixture was treated with ethyl acetate and water. The organic layer was separated, washed with water, dried over MgSO₄ and concentrated. The residue was chromatographed on silica gel using n-hexane/ethyl acetate as an eluent to give the title compound as crystals (0.50 g).

mp. 217-219° C. (ethyl acetate).

¹H-NMR (300 MHz, DMSO-d₆) δ: 3.80 (s, 3H), 4.57 (s, 2H), 5.42 (s, 1H), 6.95 (d, J=8.4 Hz, 1H), 7.09 (d, J=1.8 Hz, 1H), 7.15-7.25 (m, 6H), 7.35 (s, 1H), 7.58 (d, J=7.8 Hz, 1H), 7.67 (d, J=1.8 Hz, 1H), 7.72 (dd, J=7.8, 1.8 Hz, 1H), 10.75 (s, 1H).

### Example 133

### 6-{7-[(2-Hydroxypropyl)amino]-2-phenyl-2H-thiochromen-3-yl}-2H-1,4-benzoxazin-3(4H)-one

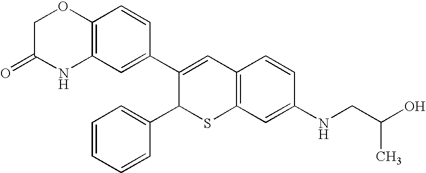

According to the method of Example 119, 6-(7-iodo-2-phenyl-2H-thiochromen-3-yl)-2H-1,4-benzoxazin-3(4H)-one (100 mg) and (±)-1-amino-2-propanol (0.10 mL) were reacted to give the title compound as a foam (22 mg).

¹H-NMR (300 MHz, CDCl₃) δ: 1.23 (d, J=8.7 Hz, 3H), 1.90-2.10 (br, 1H), 2.90-2.98 (m, 1H), 3.14-3.19 (m, 1H), 3.93-4.03 (m, 1H), 4.00-4.30 (br, 1H), 4.56 (s, 2H), 4.82 (s, 1H), 6.38-6.42 (m, 2H), 6.78-7.30 (m, 10H), 8.38 (s, 1H).

### Example 134

### 6-[7-(1-Hydroxy-1-methylethyl)-2-phenyl-2H-thiochromen-3-yl]-2H-1,4-benzoxazin-3(4H)-one

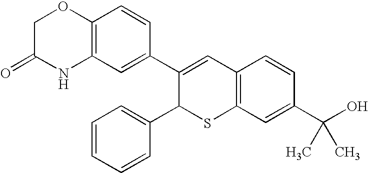

To a cooled (0° C.) solution of methyl 3-(3-oxo-3,4-dihydro-2H-1,4-benzoxazin-6-yl)-2-phenyl-2H-thiochromene-7-carboxylate (80 mg) in THF (2 mL) was added 3M methylmagnesium bromide solution in ether (0.7 mL). The mixture was stirred at 0° C. for 1 hr and at room temperature for 2 hr and quenched by the addition of 20% aqueous ammonium chloride. The organic layer was separated, and the aqueous layer was further extracted with ethyl acetate. The organic layers were combined, dried over MgSO₄ and concentrated. The residue was chromatographed on silica gel using n-hexane/ethyl acetate as an eluent to give the title compound as colorless crystals (40 mg).

mp. 241-245° C. (ethyl acetate/n-hexane).

¹H-NMR (300 MHz, CDCl₃) δ: 1.50 (s, 6H), 2.05 (s, 1H), 4.55 (s, 2H), 4.88 (s, 1H), 6.84-6.87 (m, 2H), 6.99 (dd, J=8.4, 2.1 Hz, 1H), 7.07 (s, 1H), 7.12-7.28 (m, 8H), 9.03 (s, 1H).

### Example 135

### 6-{7-[(2-Hydroxy-1,1-dimethylethyl)amino]-2-phenyl-2H-thiochromen-3-yl}-2H-1,4-benzoxazin-3(4H)-one

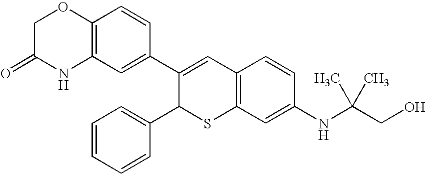

According to the method of Example 119, 6-(7-iodo-2-phenyl-2H-thiochromen-3-yl)-2H-1,4-benzoxazin-3(4H)-one (100 mg) and 2-amino-2-methyl-1-propanol (0.10 mL) were reacted to give the title compound as a foam (3 mg).

¹H-NMR (300 MHz, CD₃OD) δ: 1.29 (s, 6H), 3.36 (s, 2H), 4.57 (s, 2H), 5.21 (s, 1H), 6.92 (d, J=8.7 Hz, 1H), 7.04 (d, J=2.1 Hz, 1H), 7.16-7.30 (m, 9H), 7.54 (d, J=8.7 Hz, 1H).

### Example 136

### 3-(3-Oxo-3,4-dihydro-2H-1,4-benzoxazin-6-yl)-2-phenyl-2H-thiochromene-7-carboxylic acid

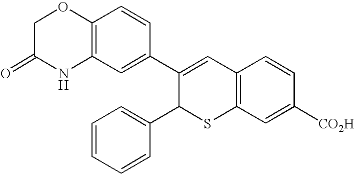

To a suspension of methyl 3-(3-oxo-3,4-dihydro-2H-1,4-benzoxazin-6-yl)-2-phenyl-2H-thiochromene-7-carboxylate (0.33 g) in ethanol (18 mL) and THF (2 mL) was added 4N NaOH (9 mL). The mixture was stirred at room temperature for 1.5 hr and concentrated. The residual mixture was adjusted to pH 1 with 2N HCl and extracted with ethyl acetate. The extract was dried over MgSO₄ and concentrated, and the residue was crystallized from THF/ethyl acetate to give the title compound as crystals (0.23 g).

¹H-NMR (300 MHz, DMSO-d₆) δ: 4.57 (s, 2H), 5.41 (s, 1H), 6.94 (d, J=8.4 Hz, 1H), 7.09 (d, J=2.1 Hz, 1H), 7.15-7.26 (m, 6H), 7.35 (s, 1H), 7.55 (d, J=8.4 Hz, 1H), 7.65 (s, 1H), 7.69 (dd, J=8.1, 1.8 Hz, 1H), 10.75 (s, 1H), 12.97 (br, 1H).

### Example 137

### 3-(3-Oxo-3,4-dihydro-2H-1,4-benzoxazin-6-yl)-2-phenyl-2H-thiochromene-7-carboxamide

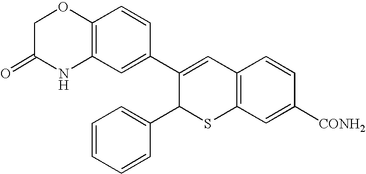

To a mixture of 3-(3-oxo-3,4-dihydro-2H-1,4-benzoxazin-6-yl)-2-phenyl-2H-thiochromene-7-carboxylic acid (80 mg) and DMF (1 drop) in THF (2 mL) was added oxalyl chloride (0.20 mL). The mixture was stirred at room temperature for 0.5 hr and concentrated. The residue was dissolved in THF and the solvent was evaporated. The residue was dissolved in THF (1 mL) and then the solution was added to a mixture of 28% ammonia solution (1 mL) and THF (1 mL). The mixture was stirred at room temperature for 64 hr and treated with ethyl acetate and brine. The organic layer was separated, dried over MgSO₄ and concentrated. The residue was chromatographed on silica gel using n-hexane/ethyl acetate as an eluent to give the title compound as a powder (13 mg).

¹H-NMR (300 MHz, DMSO-d₆) δ: 4.57 (s, 2H), 5.38 (s, 1H), 6.94 (d, J=8.7 Hz, 1H), 7.08 (d, J=2.1 Hz, 1H), 7.13-7.33 (m, 8H), 7.51 (d, J=8.4 Hz, 1H), 7.62-7.65 (m, 2H), 7.91 (brs, 1H), 10.74 (s, 1H).

### Example 138

### N-Methyl-3-(3-oxo-3,4-dihydro-2H-1,4-benzoxazin-6-yl)-2-phenyl-2H-thiochromene-7-carboxamide

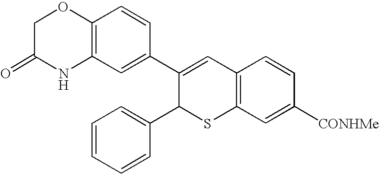

According to the method of Example 137, 3-(3-oxo-3,4-dihydro-2H-1,4-benzoxazin-6-yl)-2-phenyl-2H-thiochromene-7-carboxylic acid (80 mg) and 40% methylamine solution (1 mL) were reacted to give the title compound as a foam (15 mg).

¹H-NMR (300 MHz, CDCl₃) δ: 2.95 (d, J=4.5 Hz, 3H), 4.59 (s, 2H), 4.90 (s, 1H), 6.16 (q, J=4.5 Hz, 1H), 6.87 (d, J=8.4 Hz, 1H), 6.96 (dd, J=8.4, 2.4 Hz, 1H), 7.04-7.07 (m, 2H), 7.16-7.25 (m, 6H), 7.47 (d, J=1.8 Hz, 1H), 7.54 (dd, J=8.1, 1.8 Hz, 1H), 9.06 (s, 1H).

### Example 139

### N,N-Dimethyl-3-(3-oxo-3,4-dihydro-2H-1,4-benzoxazin-6-yl)-2-phenyl-2H-thiochromene-7-carboxamide

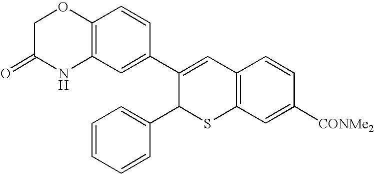

According to the method of Example 137, 3-(3-oxo-3,4-dihydro-2H-1,4-benzoxazin-6-yl)-2-phenyl-2H-thiochromene-7-carboxylic acid (80 mg) and 50% dimethylamine solution (1 mL) were reacted to give the title compound as a foam (12 mg).

¹H-NMR (300 MHz, CDCl₃) δ: 2.90-3.10 (m, 6H), 4.51 (s, 2H), 4.91 (s, 1H), 6.88 (d, J=8.4 Hz, 1H), 6.95-7.02 (m, 2H), 7.18 (s, 1H), 7.14-7.25 (m, 8H), 8.75 (s, 1H).

### Preparation 65

### 6-[(2-Fluorophenyl)acetyl]-2H-1,4-benzoxazin-3(4H)-one

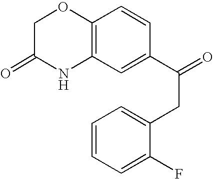

To a mixture of o-fluorophenylacetic acid (25.1 g) and DMF (1 mL) in THF (200 mL) was added dropwise oxalyl chloride (36.1 mL) under ice-cooling. The mixture was stirred at 0° C. for 1 hr and at room temperate for 0.5 hr. After concentration, the residue was dissolved in THF and the solvent was evaporated to give o-fluorophenylacetyl chloride. To a mixture of 2H-1,4-benzoxazin-3(4H)-one (18.64 g) and nitrobenzene (150 mL) was added powdered AlCl₃ (50 g) under ice-cooling, and the mixture was stirred for 10 min. To the mixture was added dropwise a solution of o-fluorophenylacetyl chloride in nitrobenzene (50 mL). After the addition was completed, the cooling-bath was removed. The mixture was stirred at room temperature for 4 hr and poured onto crashed ice. Diisopropyl ether (1 L) and 1N HCl (100 mL) were added, and the whole was stirred for 0.5 hr. Precipitate was collected by filtration, washed with water and then diisopropyl ether, and dried to give the title compound as colorless crystals (31.4 g).

¹H-NMR (300 MHz, CDCl₃) δ: 4.26 (s, 2H), 4.70 (s, 2H), 7.02-7.29 (m, 5H), 7.51 (d, J=1.8 Hz, 1H), 7.70 (dd, J=8.4, 1.8 Hz, 1H), 8.15-8.30 (br, 1H).

### Preparation 66

### 6-[Bromo(2-fluorophenyl)acetyl]-2H-1,4-benzoxazin-3(4H)-one

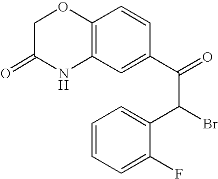

According to the method of Preparation 14, 6-[(2-fluorophenyl)acetyl]-2H-1,4-benzoxazin-3(4H)-one (20.0 g) was reacted to give the title compound as colorless crystals (21.6 g).

¹H-NMR (300 MHz, CDCl₃) δ: 4.71 (s, 2H), 6.66 (s, 1H), 6.99-7.36 (m, 4H), 7.55-7.65 (m, 3H), 8.79 (brs, 1H).

### Example 140

### 6-[2-(2-Fluorophenyl)-7-iodo-2H-thiochromen-3-yl]-2H-1,4-benzoxazin-3(4H)-one

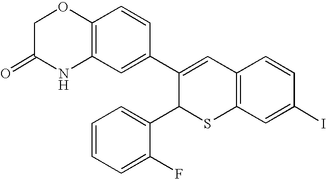

According to the method of Example 20, 6-[bromo(2-fluorophenyl)acetyl]-2H-1,4-benzoxazin-3(4H)-one (0.39 g) and (4-iodo-2-mercaptobenzyl)(triphenyl)phosphonium bromide (0.63 g) were reacted to give the title compound as colorless crystals (0.32 g).

¹H-NMR (300 MHz, DMSO-d₆) δ: 4.56 (s, 2H), 5.41 (s, 1H), 6.92-6.98 (m, 4H), 7.14 (dd, J=8.4, 2.1 Hz, 1H), 7.24-7.33 (m, 4H), 7.55-7.56 (m, 2H), 10.70 (s, 1H).

### Example 141

### 6-[2-(2-Chlorophenyl)-7-iodo-2H-thiochromen-3-yl]-2H-1,4-benzoxazin-3(4H)-one

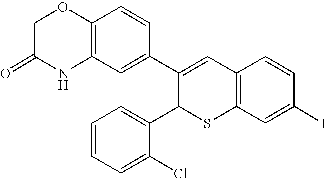

According to the method of Example 20, 6-[bromo(2-chlorophenyl)acetyl]-2H-1,4-benzoxazin-3(4H)-one (0.41 g) and (4-iodo-2-mercaptobenzyl)(triphenyl)phosphonium bromide (0.63 g) were reacted to give the title compound as colorless crystals (0.28 g).

¹H-NMR (300 MHz, DMSO-d₆) δ: 4.55 (s, 2H), 5.40 (s, 1H), 6.92-7.37 (m, 7H), 7.37 (s, 1H), 7.51-7.55 (m, 3H), 10.75 (s, 1H).

### Example 142

### 6-[2-(4-Fluorophenyl)-7-iodo-2H-thiochromen-3-yl]-2H-1,4-benzoxazin-3(4H)-one

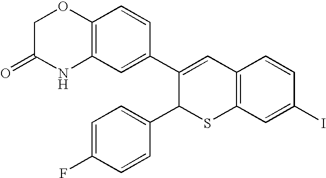

According to the method of Example 20, 6-[bromo(4-fluorophenyl)acetyl]-2H-1,4-benzoxazin-3(4H)-one (0.39 g) and (4-iodo-2-mercaptobenzyl)(triphenyl)phosphonium bromide (0.63 g) were reacted to give the title compound as colorless crystals (0.33 g).

¹H-NMR (300 MHz, DMSO-d₆) δ: 4.56 (s, 2H), 5.37 (s, 1H), 6.93 (d, J=8.4 Hz, 1H), 7.01-7.29 (m, 8H), 7.50-7.52 (m, 2H), 10.72 (s, 1H).

### Example 143

### 6-{2-(2-Fluorophenyl)-7-[(2-hydroxyethyl)amino]-2H-thiochromen-3-yl}-2H-1,4-benzoxazin-3(4H)-one

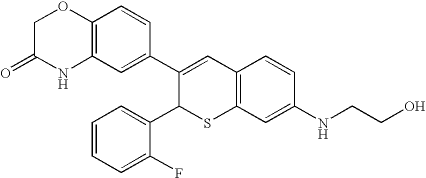

According to the method of Example 119, 6-[2-(2-fluorophenyl)-7-iodo-2H-thiochromen-3-yl]-2H-1,4-benzoxazin-3(4H)-one (93 mg) and ethanolamine (0.12 mL) were reacted to give the title compound as a foam (54 mg).

¹H-NMR (300 MHz, CDCl₃) δ: 2.30-2.70 (br, 1H), 3.18 (t, J=5.1 Hz, 2H), 3.73 (t, J=5.1 Hz, 2H), 4.00-4.40 (br, 1H), 4.52 (s, 2H), 5.22 (s, 1H), 6.33-6.39 (m, 2H), 6.78-7.13 (m, 9H), 9.09 (s, 1H).

### Example 144

### 6-{2-(2-Chlorophenyl)-7-[(2-hydroxyethyl)amino]-2H-thiochromen-3-yl}-2H-1,4-benzoxazin-3(4H)-one

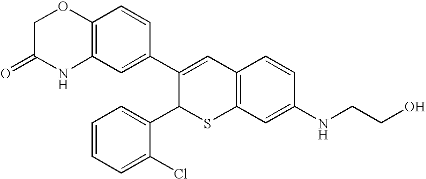

According to the method of Example 119, 6-[2-(2-chlorophenyl)-7-iodo-2H-thiochromen-3-yl]-2H-1,4-benzoxazin-3(4H)-one (90 mg) and ethanolamine (0.12 mL) were reacted to give the title compound as a foam (42 mg).

¹H-NMR (300 MHz, CDCl₃) δ: 2.00-2.30 (br, 1H), 3.23 (t, J=5.1 Hz, 2H), 3.78 (t, J=5.1 Hz, 2H), 4.00-4.40 (br, 1H), 4.56 (s, 2H), 5.35 (s, 1H), 6.37-6.43 (m, 2H), 6.73-7.42 (m, 9H), 8.46 (s, 1H).

### Example 145

### 6-{2-(4-Fluorophenyl)-7-[(2-hydroxyethyl)amino]-2H-thiochromen-3-yl}-2H-1,4-benzoxazin-3(4H)-one

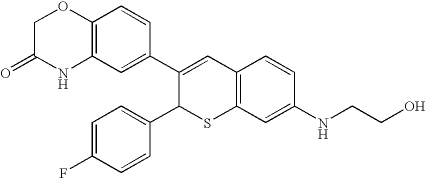

According to the method of Example 119, 6-[2-(4-fluorophenyl)-7-iodo-2H-thiochromen-3-yl]-2H-1,4-benzoxazin-3(4H)-one (93 mg) and ethanolamine (0.12 mL) were reacted to give the title compound as a foam (41 mg).

¹H-NMR (300 MHz, CDCl₃) δ: 2.20-2.50 (br, 1H), 3.20 (t, J=5.1 Hz, 2H), 3.75 (t, J=5.1 Hz, 2H), 4.54 (s, 2H), 4.79 (s, 1H), 4.00-4.40 (br, 1H), 6.37-6.40 (m, 2H), 6.78-6.95 (m, 6H), 7.06 (d, J=8.4 Hz, 1H), 7.20-7.25 (m, 2H), 9.08 (s, 1H).

### Example 146

### 6-{7-[(2-Phenoxyethyl)amino]-2-phenyl-2H-chromen-3-yl}-2H-1,4-benzoxazin-3(4H)-one

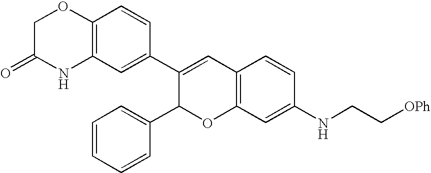

According to the method of Example 119, 6-(7-iodo-2-phenyl-2H-chromen-3-yl)-2H-1,4-benzoxazin-3(4H)-one (96 mg) and 2-phenoxyethylamine (137 mg) were reacted to give the title compound as colorless crystals (30 mg).

¹H-NMR (300 MHz, CDCl₃) δ: 3.42 (t, J=5.1 Hz, 2H), 4.07 (t, J=5.1 Hz, 2H), 4.10-4.30 (br, 1H), 4.54 (s, 2H), 6.08 (d, J=2.1 Hz, 1H), 6.13 (s, 1H), 6.17 (dd, J=8.1, 2.1 Hz, 1H), 6.79-6.96 (m, 8H), 7.20-7.28 (m, 5H), 7.39-7.42 (m, 2H), 9.10 (s, 1H).

### Example 147

### 6-(7-{[2-(2-Hydroxyethoxy)ethyl]amino}-2-phenyl-2H-chromen-3-yl)-2H-1,4-benzoxazin-3(4H)-one

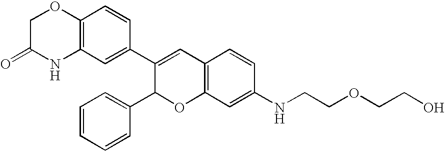

According to the method of Example 119, 6-(7-iodo-2-phenyl-2H-chromen-3-yl)-2H-1,4-benzoxazin-3(4H)-one (96 mg) and 2-(2-aminoethoxy)ethanol (105 mg) were reacted to give the title compound as a foam (37 mg).

¹H-NMR (300 MHz, CDCl₃) δ: 3.21 (t, J=5.1 Hz, 2H), 3.54 (t, J=4.8 Hz, 2H), 3.63 (t, J=5.1 Hz, 2H), 3.70 (t, J=4.8 Hz, 2H), 4.54 (s, 2H), 6.05 (d, J=2.1 Hz, 1H), 6.12-6.16 (m, 2H), 6.75 (d, J=2.1 Hz, 1H), 6.83 (d, J=8.4 Hz, 1H), 6.90-6.92 (m, 3H), 7.21-7.27 (m, 3H), 7.38-7.41 (m, 2H), 8.87 (s, 1H).

### Example 148

### 6-[7-(Octylamino)-2-phenyl-2H-chromen-3-yl]-2H-1,4-benzoxazin-3(4H)-one

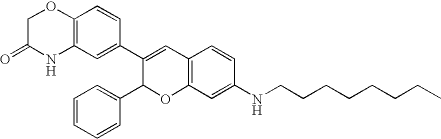

According to the method of Example 119, 6-(7-iodo-2-phenyl-2H-chromen-3-yl)-2H-1,4-benzoxazin-3(4H)-one (96 mg) and octylamine (115 mg) were reacted to give the title compound as a foam (4 mg).

¹H-NMR (300 MHz, CDCl₃) δ: 0.88 (t, J=7.2 Hz, 3H), 1.20-1.40 (m, 10H), 1.40-1.70 (m, 2H), 3.03 (t, J=7.2 Hz, 2H), 4.58 (s, 2H), 6.02 (d, J=2.1 Hz, 1H), 6.11-6.14 (m, 2H), 6.74 (d, J=2.1 Hz, 1H), 6.86-6.98 (m, 4H), 7.25-7.30 (m, 3H), 7.40-7.44 (m, 2H), 7.79 (brs, 1H).

### Preparation 67

### Ethyl 4-Hydroxy-2-methyl-1,3-thiazole-5-carboxylate

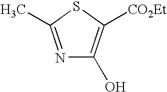

A mixture of ethyl bromomalonate (48.8 g) and thioacetamide (15.3 g) in toluene (200 mL) was refluxed for 4 hr and then cooled. The insoluble material was filtered off and the filtrate was concentrated. The residue was suspended in diisopropyl ether and collected by filtration to give the title compound (10.8 g).

mp. 104° C.

¹H-NMR (300 MHz, CDCl₃) δ: 1.37 (t, J=7.2 Hz, 3H), 2.67 (s, 3H), 4.35 (q, J=7.2 Hz, 2H).

### Preparation 68

### Ethyl 4-{[(Dimethylamino)carbonothioyl]oxy}-2-methyl-1,3-thiazole-5-carboxylate

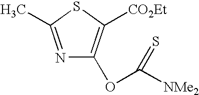

According to the method of Preparation 60, ethyl 4-hydroxy-2-methyl-1,3-thiazole-5-carboxylate (5.98 g) was reacted to give the title compound as a oil (8.60 g).

¹H-NMR (300 MHz, CDCl₃) δ: 1.32 (t, J=7.2 Hz, 3H), 2.71 (s, 3H), 3.38 (s, 3H), 3.45 (s, 3H), 4.28 (q, J=7.2 Hz, 2H).

### Preparation 69

### Ethyl 4-{[(Dimethylamino)carbonyl]thio}-2-methyl-1,3-thiazole-5-carboxylate

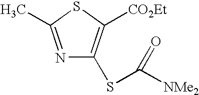

A mixture of ethyl 4-{[(dimethylamino)carbonothioyl]oxy}-2-methyl-1,3-thiazole-5-carboxylate (8.60 g) and diphenyl ether (50 mL) was heated at 190° C. for 6 hr, cooled, and chromatographed on silica gel using n-hexane/ethyl acetate as an eluent to give the title compound as crystals (6.18 g).

¹H-NMR (300 MHz, CDCl₃) δ: 1.34 (t, J=7.2 Hz, 3H), 2.73 (s, 3H), 3.00-3.20 (m, 6H), 4.32 (q, J=7.2 Hz, 2H).

### Preparation 70

### Ethyl 4-Mercapto-2-methyl-1,3-thiazole-5-carboxylate

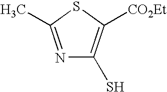

To a mixture of ethyl 4-{[(dimethylamino)carbonyl]thio}-2-methyl-1,3-thiazole-5-carboxylate (3.00 g) in methanol (100 mL) was added NaH (60% oil dispersion, 1.44 g). The mixture was refluxed for 1 hr under a nitrogen atmosphere and concentrated. The residue was treated with 1N HCl and extracted with ethyl acetate. The extract was washed with brine, dried over MgSO₄ and concentrated to give the title compound, which was used for the next step without further purification.

### Preparation 71

### (4-Mercapto-2-methyl-1,3-thiazol-5-yl)methanol

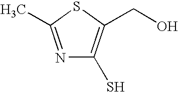

To a cooled (0° C.) suspension of LiAlH₄ (0.68 g) in THF (100 mL) was added a solution of ethyl 4-mercapto-2-methyl-1,3-thiazole-5-carboxylate in THF (50 mL). The mixture was stirred at room temperature for 2 hr and quenched by the addition of water. Saturated aqueous potassium sodium (+)-tartrate was added and the whole was stirred for an additional 3 hr. The mixture was adjusted to pH 4-5 by the addition of 1N HCl, salted out by the addition of NaCl and extracted with THF (2×). The extracts were combined, dried over MgSO₄ and concentrated to give the title compound, which was used for the next step without further purification.

### Preparation 72

### 6-[{[5-(Hydroxymethyl)-2-methyl-1,3-thiazol-4-yl]thio}(phenyl)acetyl]-2H-1,4-benzoxazin-3(4H)-one

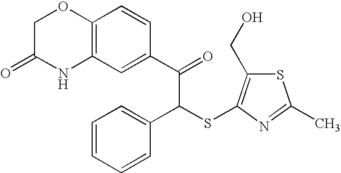

According to the method of Preparation 55, 6-[bromo(phenyl)acetyl]-2H-1,4-benzoxazin-3(4H)-one (1.80 g) and (4-mercapto-2-methyl-1,3-thiazol-5-yl)methanol were reacted to give the title compound as a foam (0.65 g).

¹H-NMR (300 MHz, CDCl₃) δ: 2.24 (t, J=6.6 Hz, 1H), 2.65 (s, 3H), 4.42 (dd, J=13.2, 6.9 Hz, 1H), 4.61-4.68 (m, 3H), 6.17 (s, 1H), 6.90 (d, J=8.7 Hz, 1H), 7.25-7.32 (m, 5H), 7.50 (d, J=2.1 Hz, 1H), 7.55 (dd, J=8.7, 2.1 Hz, 1H), 8.42 (brs, 1H).

### Preparation 73

### 6-[{[5-(Bromomethyl)-2-methyl-1,3-thiazol-4-yl]thio}(phenyl)acetyl]-2H-1,4-benzoxazin-3(4H)-one

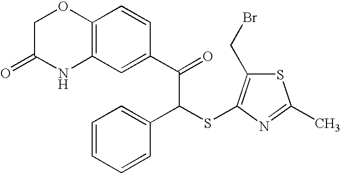

According to the method of Preparation 56, 6-[{[5-(hydroxymethyl)-2-methyl-1,3-thiazol-4-yl]thio}(phenyl)acetyl]-2H-1,4-benzoxazin-3(4H)-one (0.63 g) was reacted to give the title compound as a foam (0.34 g).

¹H-NMR (300 MHz, CDCl₃) δ: 2.59 (s, 3H), 4.34 (d, J=11.4 Hz, 1H), 4.41 (d, J=11.4 Hz, 1H), 4.60 (s, 2H), 6.23 (s, 1H), 6.85 (d, J=8.7 Hz, 1H), 7.16-7.73 (m, 7H), 10.01 (s, 1H).

### Preparation 74

### [(2-Methyl-4-{[2-oxo-2-(3-oxo-3,4-dihydro-2H-1,4-benzoxazin-6-yl)-1-phenylethyl]thio}-1,3-thiazol-5-yl)methyl](triphenyl)phosphonium Bromide

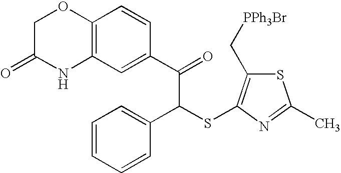

According to the method of Preparation 57, 6-[{[5-(bromomethyl)-2-methyl-1,3-thiazol-4-yl]thio}(phenyl)acetyl]-2H-1,4-benzoxazin-3(4H)-one (0.34 g) was reacted to give the title compound as colorless crystals (0.40 g).

¹H-NMR (300 MHz, CDCl₃) δ: 2.37 (s, 3H), 4.56 (d, J=15.6 Hz, 1H), 4.59 (d, J=15.6 Hz, 1H), 5.56 (dd, J=15.6, 13.8 Hz, 1H), 6.01 (s, 1H), 6.19 (dd, J=15.6, 13.8 Hz, 1H), 6.83 (d, J=8.7 Hz, 1H), 7.18-7.82 (m, 21H), 8.00 (d, J=2.1 Hz, 1H), 9.74 (s, 1H).

### Example 149

### 6-(2-Methyl-5-phenyl-5H-thiopyrano[2,3-d][1,3]thiazol-6-yl)-2H-1,4-benzoxazin-3(4H)-one

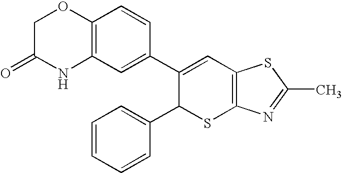

According to the method of Example 126, [(2-methyl-4-{[2-oxo-2-(3-oxo-3,4-dihydro-2H-1,4-benzoxazin-6-yl)-1-phenylethyl]thio}-1,3-thiazol-5-yl)methyl](triphenyl)phosphonium bromide (0.39 g) was reacted to give the title compound as a foam (0.02 g).

¹H-NMR (300 MHz, CDCl₃) δ: 2.63 (s, 3H), 4.60 (s, 2H), 5.03 (s, 1H), 6.86-6.89 (m, 2H), 6.98 (dd, J=8.7, 2.1 Hz, 1H), 7.04 (s, 1H), 7.17-7.37 (m, 5H), 9.15 (s, 1H).

### Example 150

### 6-(7-Iodo-2H-thiochromen-3-yl)-2H-1,4-benzoxazin-3(4H)-one

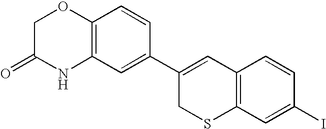

According to the method of Example 20, 6-(chloroacetyl)-2H-1,4-benzoxazin-3(4H)-one (1.58 g) and (4-iodo-2-mercaptobenzyl)(triphenyl)phosphonium bromide (4.14 g) were reacted to give the title compound as a powder (1.78 g).

¹H-NMR (300 MHz, DMSO-d₆) δ: 3.83 (s, 2H), 4.60 (s, 2H), 6.80 (s, 1H), 6.98 (d, J=8.4 Hz, 1H), 7.04-7.08 (m, 2H), 7.18 (dd, J=8.4, 2.1 Hz, 1H), 7.47 (dd, J=7.8, 1.8 Hz, 1H), 7.61 (d, J=1.8 Hz, 1H), 10.74 (s, 1H).

### Example 151

### 6-{7-[(2-Hydroxyethyl)amino]-2H-thiochromen-3-yl}-2H-1,4-benzoxazin-3(4H)-one

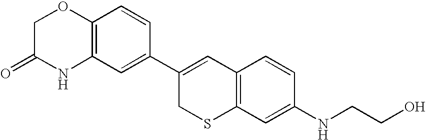

According to the method of Example 119, 6-(7-iodo-2H-thiochromen-3-yl)-2H-1,4-benzoxazin-3(4H)-one (168 mg) and ethanolamine (0.30 mL) were reacted to give the title compound as a powder (50 mg).

¹H-NMR (300 MHz, DMSO-d₆) δ: 3.09 (q, J=6.0 Hz, 2H), 3.53 (q, J=6.0 Hz, 2H), 3.73 (s, 2H), 4.57 (s, 2H), 4.69 (t, J=6.0 Hz, 1H), 5.89 (t, J=6.0 Hz, 1H), 6.37 (dd, J=8.4, 2.4 Hz, 1H), 6.44 (d, J=2.4 Hz, 1H), 6.65 (s, 1H), 6.92-7.10 (m, 4H), 10.67 (brs, 1H).

### Example 152

### 6-{7-[(2-Methoxyethyl)amino]-2H-thiochromen-3-yl}-2H-1,4-benzoxazin-3(4H)-one

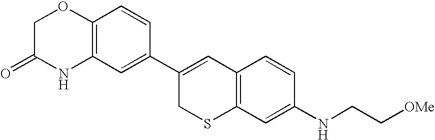

According to the method of Example 119, 6-(7-iodo-2H-thiochromen-3-yl)-2H-1,4-benzoxazin-3(4H)-one (168 mg) and 2-methoxyethylamine (0.30 mL) were reacted to give the title compound as a powder (50 mg).

¹H-NMR (300 MHz, DMSO-d₆) δ: 3.19 (q, J=5.7 Hz, 2H), 3.27 (s, 3H), 3.46 (t, J=5.7 Hz, 2H), 3.72 (s, 2H), 4.57 (s, 2H), 5.94 (t, J=5.7 Hz, 1H), 6.39 (dd, J=8.4, 2.4 Hz, 1H), 6.45 (d, J=2.4 Hz, 1H), 6.65 (s, 1H), 6.92-7.10 (m, 4H), 10.67 (s, 1H).

### Preparation 75

### 6-[2-Phenyl-7-(4,4,5,5-tetramethyl-1,3,2-dioxaborolan-2-yl)-2H-thiochromen-3-yl]-2H-1,4-benzoxazin-3(4H)-one

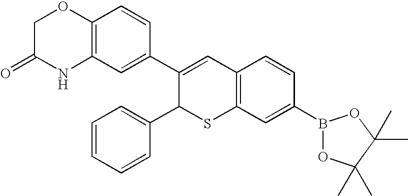

A mixture of 6-(7-iodo-2-phenyl-2H-thiochromen-3-yl)-2H-1,4-benzoxazin-3(4H)-one (1.80 g), bis(pinacolato)diboron (1.09 g), palladium(II) acetate (0.08 g), potassium acetate (1.08 g) and DMF (20 mL) was heated at 90° C. for 6 hr under an argon atmosphere. The mixture was diluted with water and extracted with ethyl acetate (2×). The extracts were combined, washed with water, dried over MgSO₄ and concentrated to give the title compound (crude, 2.00 g).

¹H-NMR (300 MHz, CDCl₃) δ: 1.30 (m, 12H), 4.59 (s, 2H), 4.85 (s, 1H), 6.83 (d, J=2.1 Hz, 1H), 6.89 (d, J=8.7 Hz, 1H), 7.05 (dd, J=8.7, 2.1 Hz, 1H), 7.10 (s, 1H), 7.17-7.28 (m, 6H), 7.54 (d, J=7.8 Hz, 1H), 7.59 (s, 1H), 7.98 (br, 1H).

### Example 153

### 6-(7-Hydroxy-2-phenyl-2H-thiochromen-3-yl)-2H-1,4-benzoxazin-3(4H)-one

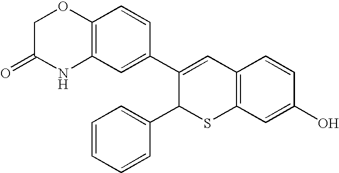

To a mixture of 6-[2-phenyl-7-(4,4,5,5-tetramethyl-1,3,2-dioxaborolan-2-yl)-2H-thiochromen-3-yl]-2H-1,4-benzoxazin-3(4H)-one (1.80 g), THF (10 mL) and acetone (10 mL) was added a solution of Oxone® (2.09 g) in water (10 mL). The mixture was stirred at room temperature for 2 hr, diluted with 10% aqueous Na₂SO₃, and extracted with THF/ethyl acetate (1/1). The extract was dried over MgSO₄ and concentrated, and the residue was chromatographed on silica gel using ethyl acetate/methanol as an eluent. The product, was suspended in diisopropyl ether and collected by filtration to give the title compound as a powder (0.80 g).

¹H-NMR (300 MHz, DMSO-d₆) δ: 4.56 (s, 2H), 5.65 (s, 1H), 6.92-7.27 (m, 10H), 7.41 (s, 1H), 7.54 (d, J=8.4 Hz, 1H), 10.18 (brs, 1H), 10.73 (s, 1H).

### Example 154

### 6-(2-Phenyl-7-pyridin-2-yl-2H-thiochromen-3-yl)-2H-1,4-benzoxazin-3(4H)-one

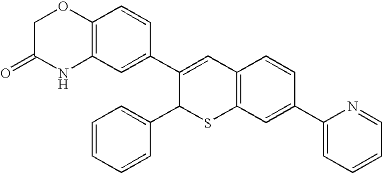

A mixture of 6-[2-phenyl-7-(4,4,5,5-tetramethyl-1,3,2-dioxaborolan-2-yl)-2H-thiochromen-3-yl]-2H-1,4-benzoxazin-3(4H)-one (100 mg), 2-bromopyridine (38 mg), [1,1′-bis(diphenylphosphino)ferrocene]dichloropalladium(II) dichloromethane adduct (33 mg), 2M cesium carbonate (0.5 mL) and THF (3 mL) was heated at 90° C. for 12 hr under a nitrogen atmosphere. The mixture was diluted with water and extracted with ethyl acetate. The extract was dried over MgSO₄ and concentrated, and the residue was chromatographed on silica gel using n-hexane/ethyl acetate as an eluent to give the title compound as crystals (42 mg).

¹H-NMR (300 MHz, CDCl₃) δ: 4.58 (s, 2H), 4.89 (s, 1H), 6.85-6.89 (m, 2H), 7.01 (dd, J=8.4, 2.8 Hz, 1H), 7.05 (s, 1H), 7.15-7.28 (m, 6H), 7.34 (d, J=8.4 Hz, 1H), 7.62-7.80 (m, 4H), 8.65-8.67 (m, 1H), 9.28 (brs, 1H).

### Preparation 76

### 4-Methyl-2-nitropyridin-3-ol

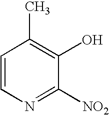

4-Methylpyridin-3-ol (5.00 g, J. Heterocyclic Chem., 1985, 22, 1419) was added to conc. H₂SO₄ (25 mL) under ice-cooling (below 30° C.). Nitric acid (fuming, 2.2 mL) was added dropwise below 10° C., and the mixture was stirred at 10-20° C. for 2 hr and then poured onto crashed ice. The mixture was adjusted to pH 2 by the addition of 8N NaOH and extracted with ethyl acetate (2×). The extracts were combined, dried over MgSO₄ and concentrated, and the residue was chromatographed on silica gel using n-hexane/ethyl acetate as an eluent to give the title compound as yellow crystals (4.89 g).

mp. 87-88° C.

¹H-NMR (300 MHz, DMSO-d₆) δ: 2.31 (s, 3H), 7.56 (d, J=4.2 Hz, 1H), 7.93 (d, J=4.2 Hz, 1H), 10.55 (br, 1H).

### Preparation 77

### Ethyl [(6-Bromo-4-methyl-2-nitropyridin-3-yl)oxy]acetate

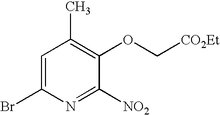

To a solution of 4-methyl-2-nitropyridin-3-ol (4.85 g) in methanol (90 mL) was added 28% sodium methoxide solution in methanol (6.3 mL). The solution was stirred at room temperature for 15 min and then cooled with an ice-bath. A solution of bromine (1.6 mL) in methanol (15 mL) was added dropwise, and the reaction mixture was stirred at 0° C. for 2 hr and concentrated to give crude 6-bromo-4-methyl-2-nitropyridin-3-ol, which was used for the next step without further purification. To a mixture of crude 6-bromo-4-methyl-2-nitropyridin-3-ol and potassium carbonate (8.70 g) in acetone (70 mL) was added ethyl bromoacetate (3.5 mL). The mixture was refluxed for 15 hr and the solvent was evaporated. DMSO (50 mL), potassium carbonate (5.00 g) and ethyl bromoacetate (1.5 mL) were additionally added, and the mixture was stirred at room temperature for 60 hr, poured into water and extracted with ethyl acetate. The extract was washed with 5% aqueous Na₂S₂O₃, water and saturated aqueous NaHCO₃, dried over MgSO₄ and concentrated. The residue was chromatographed on silica gel using n-hexane/ethyl acetate as an eluent to give the title compound as an oil (7.40 g).

¹H-NMR (300 MHz, CDCl₃) δ: 1.31 (t, J=6.6 Hz, 3H), 2.47 (s, 3H), 4.27 (q, J=6.6 Hz, 2H), 4.60 (s, 2H), 7.59 (s, 1H).

### Preparation 78

### 6-Bromo-8-methyl-2H-pyrido[3,2-b][1,4]oxazin-3(4H)-one

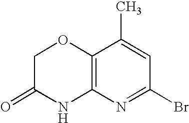

A mixture of ethyl [(6-bromo-4-methyl-2-nitropyridin-3-yl)oxy]acetate (7.40 g), iron (6.48 g), CaCl₂ (1.29 g), ethanol (150 mL) and water (35 mL) was heated at reflux for 8 hr. The insoluble material was filtered off and the filtered cake was washed with THF. The filtrate was concentrated, and the residue was treated with ethyl acetate and 1N HCl. The organic layer was separated, washed with saturated aqueous NaHCO₃, dried over MgSO₄, passed through silica gel pad and concentrated to give the title compound as colorless crystals (4.95 g).

mp. 174° C. (AcOEt/n-hexane).

¹H-NMR (300 MHz, CDCl₃) δ: 2.23 (s, 3H), 4.67 (s, 2H), 6.98 (s, 1H), 8.17 (br, 1H).

### Preparation 79

### Methyl 8-Methyl-3-oxo-3,4-dihydro-2H-pyrido[3,2-b][1,4]oxazine-6-carboxylate

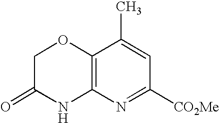

According to the method of Example 132, 6-bromo-8-methyl-2H-pyrido[3,2-b][1,4]oxazin-3(4H)-one (0.24 g) was reacted to give the title compound as a powder (0.22 g).

¹H-NMR (300 MHz, CDCl₃) δ: 2.23 (s, 3H), 3.82 (s, 3H), 4.75 (s, 2H), 7.63 (s, 1H), 11.49 (s, 1H).

### Preparation 80

### 8-Methyl-3-oxo-3,4-dihydro-2H-pyrido[3,2-b][1,4]oxazine-6-carboxylic Acid

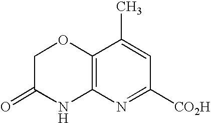

According to the method of Example 136, methyl 8-methyl-3-oxo-3,4-dihydro-2H-pyrido[3,2-b][1,4]oxazine-6-carboxylate (0.22 g) was reacted to give the title compound as a powder (0.15 g).

¹H-NMR (300 MHz, DMSO-d₆) δ: 2.22 (s, 3H), 4.73 (s, 2H), 7.60 (s, 1H), 11.42 (s, 1H), 12.88 (s, 1H).

### Preparation 81

### N-Methoxy-N,8-dimethyl-3-oxo-3,4-dihydro-2H-pyrido[3,2-b][1,4]oxazine-6-carboxamide

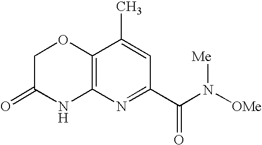

A mixture of 8-methyl-3-oxo-3,4-dihydro-2H-pyrido[3,2-b][1,4]oxazine-6-carboxylic acid (0.15 g), N,O-dimethylhydroxylamine hydrochloride (0.15 g), WSC (0.16 g), HOBt (0.13 g), triethylamine (0.35 mL) and DMF (4 mL) was stirred at room temperature for 12 hr. The mixture was treated with saturated aqueous NaHCO₃ and extracted with ethyl acetate (2×). The extracts were combined, washed with brine, dried over MgSO₄ and concentrated. The residue was suspended in ethyl acetate and collected by filtration to give the title compound as a powder (0.09 g).

¹H-NMR (300 MHz, DMSO-d₆) δ: 2.21 (s, 3H), 3.24 (s, 3H), 3.69 (s, 3H), 4.72 (s, 2H), 7.13 (s, 1H), 11.29 (s, 1H).

### Preparation 82

### 6-[(4-Fluorophenyl)acetyl]-8-methyl-2H-pyrido[3,2-b][1,4]oxazin-3(4H)-one

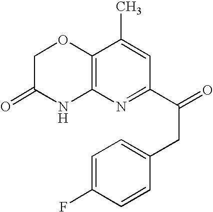

To a suspension of N-methoxy-N,8-dimethyl-3-oxo-3,4-dihydro-2H-pyrido[3,2-b][1,4]oxazine-6-carboxamide (0.09 g) in THF (9 mL) was added 0.25 M 4-fluorobenzylmagnesium chloride solution in THF (5 mL) under ice-cooling. The mixture was stirred at 0° C. for 1 hr and quenched with saturated aqueous ammonium chloride solution. The organic layer was separated, and the aqueous layer was further extracted with ethyl acetate. The organic layers were combined, dried over MgSO₄ and concentrated. The residue was suspended in diisopropyl ether and collected by filtration to give the title compound as colorless crystals (0.08 g).

¹H-NMR (300 MHz, CDCl₃) δ: 2.29 (s, 3H), 4.34 (s, 2H), 4.78 s, 2H), 6.99 (t, J=8.7 Hz, 2H), 7.22-7.26 (m, 2H), 7.64 (s, 1H), 8.03 (br, 1H).

### Preparation 83

### 6-[Bromo(4-fluorophenyl)acetyl]-8-methyl-2H-pyrido[3,2-b][1,4]oxazin-3(4H)-one

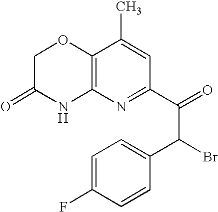

According to the method of Preparation 14, 6-[(4-fluorophenyl)acetyl]-8-methyl-2H-pyrido[3,2-b][1,4]oxazin-3(4H)-one (0.08 g) was reacted to give the title compound as a foam (0.10 g).

¹H-NMR (300 MHz, CDCl₃) δ: 2.27 (s, 3H), 4.78 (s, 2H), 6.94-7.03 (m, 3H), 7.55-7.62 (m, 2H), 7.70 (s, 1H), 8.75 (s, 1H).

### Example 155

### 6-[7-(4-Fluorophenyl)-7H-[1,2,4]triazolo[3,4-b][1,3,4]thiadiazin-6-yl]-8-methyl-2H-pyrido[3,2-b][1,4]oxazin-3(4H)-one

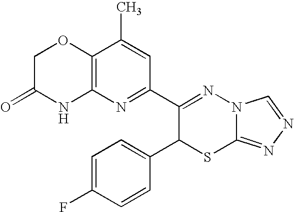

A mixture of 6-[bromo(4-fluorophenyl)acetyl]-8-methyl-2H-pyrido[3,2-b][1,4]oxazin-3(4H)-one (0.09 g) and 4-amino-3-mercapto-4H-1,2,4-triazole (0.03 g) in ethanol/toluene (1/1, 4 mL) was refluxed for 11 hr. After cooled, the precipitate was collected by filtration and washed with ethanol to give the title compound as colorless crystals (0.03 g).

mp. 256-257° C.

¹H-NMR (300 MHz, DMSO-d₆) δ: 2.26 (s, 3H), 4.75 (s, 2H), 6.42 (s, 1H), 7.09-7.25 (m, 4H), 7.85 (s, 1H), 9.24 (s, 1H), 11.41 (br, 1H).

### Preparation 84

### 6-[(4-Bromophenyl)acetyl]-2H-1,4-benzoxazin-3(4H)-one

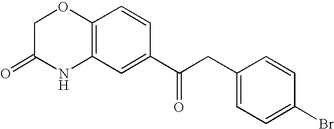

The title compound was obtained as crystals (14.7 g) from 2H-1,4-benzoxazin-3(4H)-one (8.0 g) and 4-bromophenylacetic acid according to a method similar to the procedure for 6-[(3-chlorophenyl)acetyl]-2H-1,4-benzoxazin-3(4H)-one.

¹H-NMR (300 MHz, DMSO-d₆) δ: 4.31 (s, 2H), 4.69 (s, 2H), 7.06 (d, J=8.3 Hz, 1H), 7.16-7.26 (m, 2H), 7.46-7.55 (m, 3H), 7.72 (dd, J=8.3, 1.9 Hz, 1H), 10.88 (s, 1H).

### Preparation 85

### 6-[(3-Bromophenyl)acetyl]-2H-1,4-benzoxazin-3(4H)-one

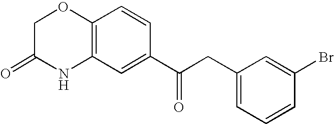

To a solution of 3-bromophenylacetic acid (12.7 g) and DMF (5 drops) in THF (200 mL) was added oxalyl chloride (8.0 mL) at room temperature, and the mixture was stirred for 1 hr at room temperature. The mixture was concentrated in vacuo to give 3-bromophenylacetyl chloride. Aluminum chloride (16.0 g) was added to a suspension of 2H-1,4-benzoxazin-3(4H)-one (8.0 g) in nitrobenzene (80 mL) under ice-cooling, and then 3-bromophenylacetyl chloride obtained above was added to the mixture under ice-cooling. The reaction mixture was allowed to warm to room temperature and stirred for 12 hr, and then poured into ice-cooled water (200 mL). Diisopropyl ether (240 mL) was added to the mixture, and the resulting crystals were collected by filtration. The crystals were suspended in methanol (100 mL) and the mixture was refluxed for 2 hr. After cooling the mixture, the resulting crystals were collected by filtration. The title compound was obtained as crystals (11.9 g).

¹H-NMR (300 MHz, DMSO-d₆) δ: 4.34 (s, 2H), 4.69 (s, 2H), 7.07 (d, J=8.6 Hz, 1H), 7.21-7.33 (m, 2H), 7.41-7.50 (m, 2H), 7.52 (d, J=2.0 Hz, 1H), 7.73 (dd, J=8.6, 2.0 Hz, 1H), 10.89 (s, 1H).

### Preparation 86

### 6-[(2-Bromophenyl)acetyl]-2H-1,4-benzoxazin-3(4H)-one

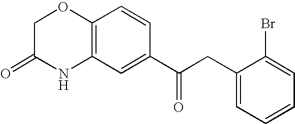

The title compound was obtained as crystals (36.9 g) from 2H-1,4-benzoxazin-3(4H)-one (16 g) and 2-bromophenylacetic acid according to a method similar to the procedure for 6-[(3-bromophenyl)acetyl]-2H-1,4-benzoxazin-3(4H)-one.

¹H-NMR (300 MHz, DMSO-d₆) δ: 4.48 (s, 2H), 4.70 (s, 2H), 7.10 (d, J=8.4 Hz, 1H), 7.18-7.28 (m, 1H), 7.33-7.40 (m, 2H), 7.55 (d, J=2.0 Hz, 1H), 7.62 (d, J=7.6 Hz, 1H), 7.77 (dd, J=8.4, 2.0 Hz, 1H), 10.89 (s, 1H).

### Preparation 87

### 6-[(4-Nitrophenyl)acetyl]-2H-1,4-benzoxazin-3(4H)-one

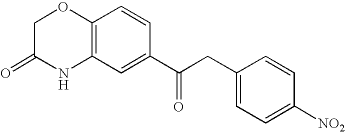

The title compound was obtained as crystals (13.7 g) from 2H-1,4-benzoxazin-3(4H)-one (8.0 g) and 4-nitrophenylacetic acid according to a method similar to the procedure for 6-[(3-chlorophenyl)acetyl]-2H-1,4-benzoxazin-3(4H)-one.

¹H-NMR (300 MHz, DMSO-d₆) δ: 4.54 (s, 2H), 4.70 (s, 2H), 7.09 (d, J=8.3 Hz, 1H), 7.46-7.60 (m, 3H), 7.75 (dd, J=8.3, 1.9 Hz, 1H), 8.19 (d, J=8.3 Hz, 2H), 10.90 (s, 1H).

### Preparation 88

### 6-[(3-Nitrophenyl)acetyl]-2H-1,4-benzoxazin-3(4H)-one

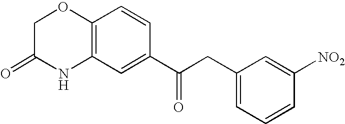

The title compound was obtained as crystals (15.4 g) from 2H-1,4-benzoxazin-3(4H)-one (7.5 g) and 3-nitrophenylacetic acid according to a method similar to the procedure for 6-[(3-bromophenyl)acetyl]-2H-1,4-benzoxazin-3(4H)-one.

¹H-NMR (300 MHz, DMSO-d₆) δ: 4.56 (s, 2H), 4.70 (s, 2H), 7.10 (d, J=8.7 Hz, 1H), 7.54 (d, J=1.9 Hz, 1H), 7.63 (t, J=8.0 Hz, 1H), 7.69-7.80 (m, 2H), 8.09-8.21 (m, 2H), 10.91 (s, 1H).

### Preparation 89

### 6-[(2-Methylphenyl)acetyl]-2H-1,4-benzoxazin-3(4H)-one

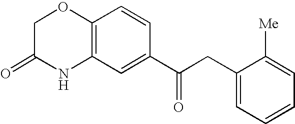

To a solution of 2-methylphenylacetic acid (8.9 g) in THF (200 mL) was added DMF (5 drops) and then oxalyl chloride (8.0 mL) was added at room temperature, and the mixture was stirred for 1 hr. The mixture was concentrated in vacuo to give 2-methylphenylacetyl chloride. Aluminum chloride (16.0 g) was added to a suspension of 2H-1,4-benzoxazin-3(4H)-one (8.0 g) in nitrobenzene (80 mL) under ice-cooling and then 2-methylphenylacetyl chloride obtained above was added. The reaction mixture was allowed to warm to room temperature and stirred for 12 hr. The mixture was poured into ice-cooled water (200 mL). The mixture was extracted with ethyl acetate. The organic layer was washed with water, saturated aqueous sodium bicarbonate solution and water, dried over Na₂SO₄ and concentrated in vacuo. The residue was crystallized from toluene to give the title compound as crystals (3.8 g).

¹H-NMR (300 MHz, DMSO-d₆) δ: 2.15 (s, 3H), 4.33 (s, 2H), 4.69 (s, 2H), 7.04-7.21 (m, 5H), 7.53 (d, J=2.2 Hz, 1H), 7.75 (dd, J=8.5, 2.2 Hz, 1H), 10.88 (s, 1H).

### Preparation 90

### Methyl 4-(2-methoxy-2-oxoethoxy)-3-nitrobenzoate

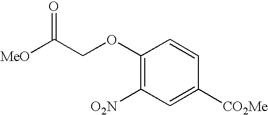

To a mixture of methyl 4-hydroxy-3-nitrobenzoate (52.2 g), potassium carbonate (60.0 g) and DMSO (250 mL) was added methyl bromoacetate (42.6 g) at room temperature, and the mixture was stirred for 12 hr. Water (750 mL) and diethyl ether (500 mL) were added to the mixture, and the aqueous layer was acidified with 10% hydrochloric acid. The resulting crystals were collected by filtration, and washed with water and diethyl ether. The title compound was obtained as crystals (61.2 g).

¹H-NMR (300 MHz, CDCl₃) δ: 3.82 (s, 3H), 3.94 (s, 3H), 4.87 (s, 2H), 7.00 (d, J=8.7 Hz, 1H), 8.20 (dd, j=8.7, 2.1 Hz, 1H), 8.54 (d, J=2.1 Hz, 1H).

### Preparation 91

### Methyl 3-oxo-3,4-dihydro-2H-1,4-benzoxazine-6-carboxylate

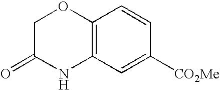

A mixture of methyl 4-(2-methoxy-2-oxoethoxy)-3-nitrobenzoate (30.0 g), iron (powder, 31.0 g), calcium chloride (6.2 g), water (75 mL) and methanol (300 mL) was refluxed for 12 hr. The mixture was passed through the Celite filter and filtrate was concentrated in vacuo. Water was added to the residue and the mixture was extracted with a mixture of ethyl acetate and THF. The organic layer was washed with water and brine, dried over Na₂SO₄ and concentrated in vacuo. The resulting crystals were washed with diisopropyl ether. The title compound was obtained as crystals (18.4 g).

¹H-NMR (300 MHz, DMSO-d₆) δ: 3.82 (s, 3H), 4.69 (s, 2H), 7.04 (d, J=8.3 Hz, 1H), 7.49-7.58 (m, 2H), 10.90 (s, 1H).

### Preparation 92

### 6-(1,3-Thiazol-2-ylacetyl)-2H-1,4-benzoxazin-3(4H)-one

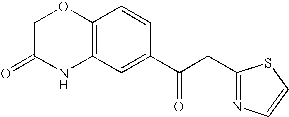

To a solution of 2-methyl-1,3-thiazole (4.8 g) in THF (50 mL) was added n-butyllithium in hexane (1.6 M, 29 mL) below −65° C. under argon atmosphere, and then the mixture was stirred for 1 hr under dry ice-acetone bath cooling. Methyl 3-oxo-3,4-dihydro-2H-1,4-benzoxazine-6-carboxylate (3.0 g) was added to the mixture, and then the mixture was allowed to warm to room temperature. The mixture was stirred for 2 hr. Water (50 mL) was added to the mixture and the aqueous layer was acidified by the addition of 10% hydrochloric acid. Organic solvents were evaporated from the mixture, and then ethyl acetate was added to the aqueous residue. Resulting crystals were collected by filtration, and washed with ethyl acetate. The crystals were suspended in methanol and the mixture was stirred for 1 hr at room temperature. The crystals were collected by filtration. The title compound was obtained as crystals (1.0 g).

¹H-NMR (300 MHz, DMSO-d₆) δ: 4.70 (s, 2H), 4.80 (s, 2H), 7.08 (d, J=8.3 Hz, 1H), 7.54 (d, J=2.3 Hz, 1H), 7.67 (d, J=3.4 Hz, 1H), 7.71-7.78 (m, 2H), 10.90 (s, 1H).

### Preparation 93

### 4-Bromo-2-methyl-6-nitrophenol

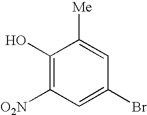

To a solution of 4-bromo-2-methylphenol (10.0 g) in acetic acid (90 mL) were added water (10 mL) and sulfuric acid (4 mL) under ice cooling. A solution of sodium nitrite (11.6 g) in water (23.2 mL) was added dropwise to the mixture below 10° C. over 1 hr, and the mixture was stirred for 1 hr. The mixture was allowed to warm to 15° C. over 1 hr, and then water (300 ml) was added to the mixture. Resulting crystals were collected by filtration, and washed with water. The title compound was obtained as crystals (9.5 g).

¹H-NMR (300 MHz, CDCl₃) δ: 2.33 (t, J=0.8 Hz, 3H), 7.53-7.58 (m, 1H), 8.10 (dd, J=2.5, 0.6 Hz, 1H), 10.82 (d, J=0.6 Hz, 1H).

### Preparation 94

### Methyl (4-bromo-2-methyl-6-nitrophenoxy)acetate

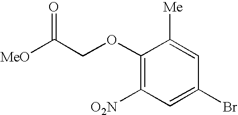

To a solution of 4-bromo-2-methyl-6-nitrophenol (9.4 g) in DMSO (50 mL) was added potassium carbonate (8.4 g) and methyl bromoacetate (6.5 g) was added dropwise to the mixture at room temperature. The mixture was stirred for 48 hr at room temperature, and then water (250 mL) was added to the mixture. The aqueous layer was acidified with 10% hydrochloric acid, and then the mixture was extracted with ethyl acetate. The organic layer was washed with water and brine, dried over Na₂SO₄ and concentrated in vacuo. Resulting crystals were collected by filtration, and washed with hexane. The title compound was obtained as crystals (9.86 g).

¹H-NMR (300 MHz, DMSO-d₆) δ: 2.34 (s, 3H), 3.70 (s, 3H), 4.67-4.73 (m, 2H), 7.85 (d, J=2.1 Hz, 1H), 8.00 (d, J=2.1 Hz, 1H).

### Preparation 95

### 6-Bromo-8-methyl-2H-1,4-benzoxazin-3(4H)-one

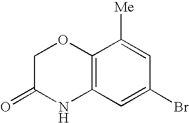

To a mixture of methyl (4-bromo-2-methyl-6-nitrophenoxy)acetate (10.9 g), acetic acid (100 mL) and THF (200 mL) was added zinc (powder, 35 g) at 45° C., and the mixture was stirred for 0.5 hr. The mixture was refluxed for 1 hr, and then filtered. The filtrate was concentrated in vacuo, and the residue was diluted with ethyl acetate. The organic layer was washed with aqueous sodium bicarbonate solution, water and brine, dried over Na₂SO₄ and concentrated in vacuo. Resulting crystals were collected by filtration, and washed with hexane. The crystals were suspended in methanol and then collected by filtration. The title compound was obtained as crystals (5.8 g).

¹H-NMR (300 MHz, DMSO-d₆) δ: 2.14 (s, 3H), 4.60 (s, 2H), 6.87 (d, J=2.3 Hz, 1H), 7.00 (d, J=2.3 Hz, 1H), 10.72 (s, 1H).

### Preparation 96

### 8-Methyl-2H-1,4-benzoxazin-3(4H)-one

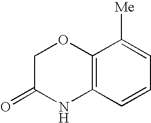

A mixture of 6-bromo-8-methyl-2H-1,4-benzoxazin-3(4H)-one (4.9 g), sodium acetate (3.3 g), 10% palladium-carbon (0.5 g), ethanol (50 mL) and THF (100 mL) was stirred under hydrogen atmosphere (3 atm) at room temperature for 6 hr. The catalyst was filtered off and the filtrate was concentrated in vacuo. Water and ethyl acetate were added to the residue and the organic layer was separated. The organic layer was washed with water and brine, dried over Na₂SO₄ and concentrated in vacuo. Resulting crystals were collected by filtration, and washed with diisopropyl ether. The title compound was obtained as crystals (3.11 g).

¹H-NMR (300 MHz, DMSO-d₆) δ: 2.15 (s, 3H), 4.56 (s, 2H), 6.69-6.88 (m, 3H), 10.61 (s, 1H).

### Preparation 97

### 6-[(4-Fluorophenyl)acetyl]-8-methyl-2H-1,4-benzoxazin-3(4H)-one

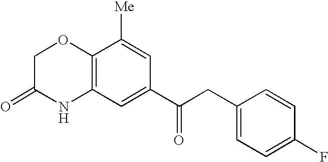

To a solution of 4-fluorophenylacetic acid (3.7 g) and DMF (3 drops) in THF (40 mL) was added oxalyl chloride (3.0 mL) under ice-cooling, and then the mixture was allowed to warm to room temperature and stirred for 1 hr. The mixture was concentrated in vacuo to give 4-fluorophenylacetyl chloride. Aluminum chloride (6.2 g) was added to a suspension of 8-methyl-2H-1,4-benzoxazin-3(4H)-one (3.0 g) in nitrobenzene (24 mL) under ice-cooling, and then 3-bromophenylacetyl chloride obtained above was added to the mixture under ice-cooling. The reaction mixture was allowed to warm to room temperature and stirred for 6 hr, and then poured into ice-cooled water (75 mL). Diisopropyl ether (75 mL) was added to the mixture, and the resulting crystals were collected by filtration, washed with diisopropyl ether. The crystals were suspended in methanol (30 mL) and the mixture was refluxed for 0.5 hr. After cooling the mixture, the resulting crystals were collected by filtration. The title compound was obtained as crystals (4.05 g).

¹H-NMR (300 MHz, DMSO-d₆) δ: 2.23 (s, 3H), 4.29 (s, 2H), 4.69 (s, 2H), 7.08-7.18 (m, 2H), 7.23-7.32 (m, 2H), 7.38 (d, J=1.7 Hz, 1H), 7.64 (d, J=1.7 Hz, 1H), 10.80 (s, 1H).

### Preparation 98

### 6-[Bromo(4-bromophenyl)acetyl]-2H-1,4-benzoxazin-3(4H)-one

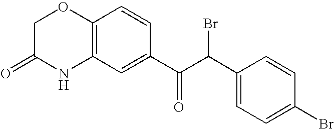

The title compound was obtained as crystals (15.1 g) from 6-[(4-bromophenyl)acetyl]-2H-1,4-benzoxazin-3(4H)-one (13 g) according to a method similar to the procedure for 6-[bromo(3-chlorophenyl)acetyl]-2H-1,4-benzoxazin-3(4H)-one.

¹H-NMR (300 MHz, DMSO-d₆) δ: 4.69 (s, 2H), 7.01-7.10 (m, 2H), 7.46-7.55 (m, 3H), 7.56-7.64 (m, 2H), 7.77 (dd, J=8.5, 2.1 Hz, 1H), 10.93 (s, 1H).

### Preparation 99

### 6-[Bromo(3-bromophenyl)acetyl]-2H-1,4-benzoxazin-3(4H)-one

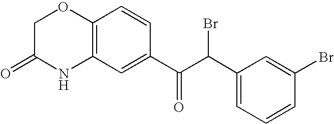

The title compound was obtained as crystals (11.6 g) from 6-[(3-bromophenyl)acetyl]-2H-1,4-benzoxazin-3(4H)-one (11.5 g) according to a method similar to the procedure for 6-[bromo(3-chlorophenyl)acetyl]-2H-1,4-benzoxazin-3(4H)-one.

¹H-NMR (300 MHz, DMSO-d₆) δ: 4.70 (s, 2H), 7.04 (s, 1H), 7.08 (d, J=8.3 Hz, 1H), 7.36 (t, J=8.0 Hz, 1H), 7.50-7.59 (m, 3H), 7.74-7.83 (m, 2H), 10.95 (s, 1H).

### Preparation 100

### 6-[Bromo(2-bromophenyl)acetyl]-2H-1,4-benzoxazin-3(4H)-one

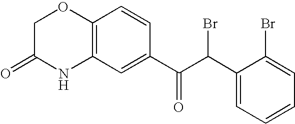

The title compound was obtained as crystals (27.9 g) from 6-[(2-bromophenyl)acetyl]-2H-1,4-benzoxazin-3(4H)-one (24.0 g) according to a method similar to the procedure for 6-[bromo(3-chlorophenyl)acetyl]-2H-1,4-benzoxazin-3(4H)-one.

¹H-NMR (300 MHz, DMSO-d₆) δ: 4.69 (s, 2H), 7.01-7.09 (m, 2H), 7.25-7.35 (m, 1H), 7.38-7.54 (m, 3H), 7.60 (dd, J=8.7, 2.3 Hz, 1H), 7.70 (dd, J=8.0, 1.1 Hz, 1H), 10.94 (s, 1H).

### Preparation 101

### 6-[Bromo(4-nitrophenyl)acetyl]-2H-1,4-benzoxazin-3(4H)-one

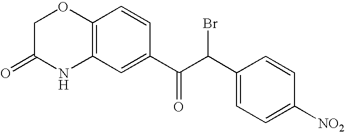

The title compound was obtained as crystals (3.58 g) from 6-[(4-nitrophenyl)acetyl]-2H-1,4-benzoxazin-3(4H)-one (3.0 g) according to a method similar to the procedure for 6-[bromo(3-chlorophenyl)acetyl]-2H-1,4-benzoxazin-3(4H)-one.

¹H-NMR (300 MHz, DMSO-d₆) δ: 4.70 (s, 2H), 7.08 (d, J=8.5 Hz, 1H), 7.20 (s, 1H), 7.54 (d, J=1.9 Hz, 1H), 7.76-7.88 (m, 3H), 8.26 (d, J=8.7 Hz, 2H), 10.95 (s, 1H).

### Preparation 102

### 6-[Bromo(3-nitrophenyl)acetyl]-2H-1,4-benzoxazin-3(4H)-one

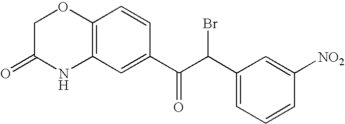

The title compound was obtained as crystals (6.1 g) from 6-[(3-nitrophenyl)acetyl]-2H-1,4-benzoxazin-3(4H)-one (5.4 g) according to a method similar to the procedure for 6-[bromo(3-chlorophenyl)acetyl]-2H-1,4-benzoxazin-3(4H)-one.

¹H-NMR (300 MHz, DMSO-d₆) δ: 4.71 (s, 2H), 7.11 (d, J=8.3 Hz, 1H), 7.21 (s, 1H), 7.56 (d, J=2.0 Hz, 1H), 7.72 (t, J=8.0 Hz, 1H), 7.85 (dd, J=8.5, 2.0 Hz, 1H), 7.98-8.06 (m, 1H), 8.18-8.27 (m, 1H), 8.48 (t, J=1.9 Hz, 1H), 10.96 (s, 1H).

### Preparation 103

### 6-[Bromo(2-methylphenyl)acetyl]-2H-1,4-benzoxazin-3(4H)-one

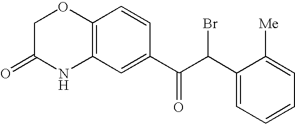

The title compound was obtained as crystals (1.74 g) from 6-[(2-methylphenyl)acetyl]-2H-1,4-benzoxazin-3(4H)-one (1.5 g) according to a method similar to the procedure for 6-[bromo(3-chlorophenyl)acetyl]-2H-1,4-benzoxazin-3(4H)-one.

¹H-NMR (300 MHz, DMSO-d₆) δ: 2.46 (s, 3H), 4.66 (s, 2H), 6.95-7.45 (m, 7H), 7.51 (dd, J=8.3, 2.3 Hz, 1H), 10.93 (s, 1H).

### Preparation 104

### 6-[Bromo(1,3-thiazol-2-yl)acetyl]-2H-1,4-benzoxazin-3(4H)-one

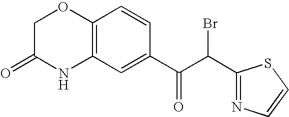

6-(1,3-Thiazol-2-ylacetyl)-2H-1,4-benzoxazin-3(4H)-one (0.9 g) was suspended in acetic acid (10 mL), and then 25% hydrogen bromide in acetic acid (2.5 mL) was added to a suspension. A solution of bromine (0.17 mL) in acetic acid (1 mL) was added to the mixture dropwise at room temperature and the mixture was stirred for 15 min. Dioxane (5 mL) and methanol (5 mL) were added to the mixture, and then bromine (0.04 mL) was added to the mixture at room temperature. After stirring, the mixture for 15 min at room temperature, the mixture was concentrated in vacuo. The residue was crystallized from the mixture of methanol and water to give the title compound as crystals (766 mg).

1H-NMR (300 MHz, DMSO-d₆) δ: 4.71 (s, 2H), 7.07 (d, J=8.5 Hz, 1H), 7.39 (s, 1H)—, 7.57 (d, J=2.3 Hz, 1H), 7.81 (dd, J=8.5, 2.3 Hz, 1H), 7.85 (d, J=3.4 Hz, 1H), 7.92 (d, J=3.4. Hz, 1H), 10.93 (s, 1H).

### Preparation 105

### 6-[Bromo(4-fluorophenyl)acetyl]-8-methyl-2H-1,4-benzoxazin-3(4H)-one

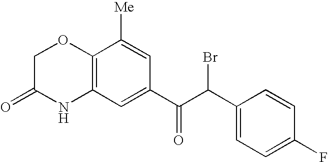

To a mixture of 6-[(4-fluorophenyl)acetyl]-8-methyl-2H-1,4-benzoxazin-3(4H)-one (2.0 g) 25% hydrogen bromide in acetic acid (7 mL) and acetic acid (21 mL) was added pyridinium hydrobromide perbromide (2.3 g) portionwise at room temperature, and the mixture was stirred for 0.5 hr. Then, aqueous sodium sulfite solution, which was prepared from sodium sulfite (0.3 g) and water (20 mL), was added dropwise to the mixture under ice-cooling, and then water (40 mL) was added dropwise to the mixture under ice-cooling. The resulting crystals were collected by filtration and washed with water. The title compound was obtained as crystals (2.48 g).

¹H-NMR (300 MHz, DMSO-d₆) δ: 2.21 (s, 3H), 4.70 (s, 2H), 7.04 (s, 1H), 7.17-7.28 (m, 2H), 7.39 (d, J=1.7 Hz, 1H), 7.57-7.66 (m, 2H), 7.72 (d, J=1.7 Hz, 1H), 10.85 (s, 1H).

### Preparation 106

### (5-Fluoro-2-mercaptobenzyl)(triphenyl)phosphonium bromide

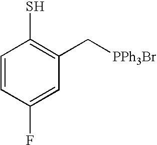

A mixture of (5-fluoro-2-mercaptophenyl)methanol (2.2 g), triphenylphosphine hydrobromide (5.0 g) and acetonitrile (30 mL) was refluxed for 4 hr under nitrogen atmosphere. After cooling the mixture to room temperature, the resulting crystals were collected by filtration (5.3 g).

¹H-NMR (300 MHz, DMSO-d₆) δ: 5.11 (d, J=15.2 Hz, 2H), 5.64 (s, 1H), 6.74-6.87 (m, 1H), 7.08-7.22 (m, 1H), 7.42 (dd, J=8.3, 6.1 Hz, 1H), 7.59-7.83 (m, 12H), 7.87-8.00 (m, 3H).

### Preparation 107

### Ethyl [(5-bromo-3-nitropyridin-2-yl)oxy]acetate

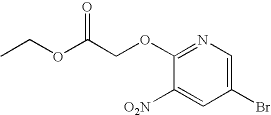

To a mixture of 5-bromo-2-chloro-3-nitropyridine (21.7 g), ethyl glycolate (11.4 g), DMF (7.5 mL) and THF (90 mL) was added sodium hydride (60% in mineral oil, 6.6 g) under ice-cooling. The mixture was allowed to warm to room temperature, and stirred for 0.5 hr. The mixture was poured into ice-cooled water and the mixture was extracted with ethyl acetate. The organic layer was washed with brine, dried over Na₂SO₄ and concentrated in vacuo. The residue was purified by chromatography on silica gel (hexane→hexane:ethyl acetate=4:1). Resulting crystals were collected by filtration, and washed with hexane. The title compound was obtained as crystals (15.8 g).

¹H-NMR (300 MHz, DMSO-d₆) δ: 1.18 (t, J=7.0 Hz, 3H), 4.14 (q, J=7.0 Hz, 2H), 5.11 (s, 2H), 8.65 (d, J=2.3 Hz, 1H), 8.77 (d, J=2.3 Hz, 1H).

### Preparation 108

### 7-Bromo-1H-pyrido[2,3-b][1,4]oxazin-2(3H)-one

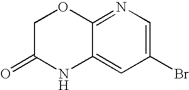

To a solution of ethyl [(5-bromo-3-nitropyridin-2-yl)oxy]acetate (15.7 g) in THF (300 mL) was added acetic acid (150 mL) at room temperature, and then the mixture was allowed to warm to 45° C. Zinc (powder, 51 g) was added to the mixture at 45° C., and the mixture was stirred at 45° C. for 0.5 hr. The mixture was filtered, and the filtrate was concentrated in vacuo. The residue was diluted with ethyl acetate, and ethyl acetate layer was washed with aqueous sodium bicarbonate solution and brine, dried over Na₂SO₄ and concentrated in vacuo. The residue was diluted with acetic acid (150 mL), and the mixture was refluxed for 1 hr. The mixture was concentrated in vacuo, and the resulting crystals were suspended in ethyl acetate. The mixture was refluxed for 2 hr, and then cooled to room temperature. The resulting crystals were collected by filtration to give the title compound (9.8 g).

¹H-NMR (300 MHz, DMSO-d₆) δ: 4.81 (s, 2H), 7.33 (d, J=2.3 Hz, 1H), 7.88 (d, J=2.3 Hz, 1H), 10.94 (s, 1H).

### Preparation 109

### (2-Oxo-2,3-dihydro-1H-pyrido[2,3-b][1,4]oxazin-7-yl)boronic acid

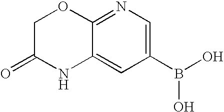

Sodium hydride (60% in mineral oil, 0.36 g) was washed with hexane and then suspended in THF (20 mL). To the suspension was added 7-bromo-1H-pyrido[2,3-b][1,4]oxazin-2(3H)-one (1.0 g) under ice cooling and the mixture was stirred under ice cooling for 0.5 hr at which time the bubbling had stopped. The mixture was cooled with dry ice-acetone bath and then n-butyllithium (1.6M in hexane, 5.5 mL) was added to the mixture below −65° C. The mixture was stirred for 0.5 hr under dry ice-acetone bath cooling and then triisopropyl borate (3.6 mL) was added below −60° C. The mixture was allowed to warm to room temperature and stirred for 0.5 hr. The mixture was poured into 2N-HCl (25 mL) under ice cooling. The mixture was allowed to warm to room temperature and stirred for 1 hr. Hexane (15 mL) was added to the mixture and the layers were separated. The aqueous layer was adjusted to pH 4 by the addition of 8N-NaOH and the resulting crystals were collected by filtration. Crystals were washed with water, hexane and dried in vacuo. The title compound was obtained as crystals (0.74 g).

¹H-NMR (300 MHz, DMSO-d₆) δ: 4.78 (s, 2H), 7.53 (d, J=1.9 Hz, 1H), 8.15 (d, J=1.9. Hz, 1H), 8.20 (s, 2H), 10.83 (s, 1H).

### Preparation 110

### 2-(2-Oxo-2,3-dihydro-1H-pyrido[2,3-b][1,4]oxazin-7-yl)-3-phenylacrylaldehyde

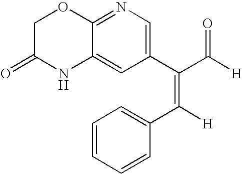

A mixture of (2-oxo-2,3-dihydro-1H-pyrido[2,3-b][1,4]oxazin-7-yl)boronic acid (0.72 g), α-bromocinnamaldehyde (1.2 g), [1,1-bis(diphenylphosphino)ferrocene]dichloropalladium(II) dichloromethane adduct (0.6 g), cesium carbonate (3.9 g), water (4 mL) and THF (20 mL) was refluxed for 12 hr under argon atmosphere. Water was added to the mixture, and the mixture was extracted with ethyl acetate. The organic layer was washed with brine, dried over Na₂SO₄ and concentrated in vacuo. The residue was purified by chromatography on silica gel (hexane→hexane:ethyl acetate=1:2) to give the crystals. The resulting crystals were washed with methanol. The title compound was obtained as crystals (0.23 g).

¹H-NMR (300 MHz, DMSO-d₆) δ: 4.84 (s, 2H), 7.03 (d, J=2.1 Hz, 1H), 7.26-7.44 (m, 5H), 7.51 (d, J=2.1 Hz, 1H), 7.79 (s, 1H), 9.74 (s, 1H), 10.86 (s, 1H).

### Preparation 111

### 8-Methyl-6-(4,4,5,5-tetramethyl-1,3,2-dioxaborolan-2-yl)-2H-1,4-benzoxazin-3(4H)-one

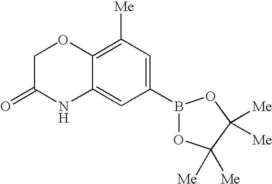

A mixture of 6-bromo-8-methyl-2H-1,4-benzoxazin-3(4H)-one (2.00 g), bis(pinacolato)diboron (2.3 g), potassium acetate (2.9 g) [1,1-bis(diphenylphosphino)ferrocene]dichloropalladium(II) dichloromethane adduct (0.34 g) and dioxane (50 mL) was stirred at 90° C. for 12 hr under argon atmosphere. Water and ethyl acetate were added to the mixture, and the organic layer was separated. The organic layer was washed with water, brine, dried over Na₂SO₄ and concentrated in vacuo. The resulting crystals were washed with diisopropyl ether. The title compound was obtained as crystals (2.36 g).

¹H-NMR (300 MHz, DMSO-d₆) δ: 1.27 (s, 12H), 2.15 (s, 3H), 4.61 (s, 2H), 7.06 (s, 1H), 7.13 ('s, 1H), 10.61 (s, 1H).

### Preparation 112

### 2-(8-Methyl-3-oxo-3,4-dihydro-2H-1,4-benzoxazin-6-yl)-3-phenylacrylaldehyde

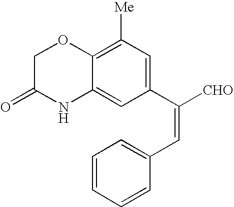

A mixture of 8-methyl-6-(4,4,5,5-tetramethyl-1,3,2-dioxaborolan-2-yl)-2H-1,4-benzoxazin-3(4H)-one (1.0 g), α-bromocinnamaldehyde (0.88 g), [1,1-bis(diphenylphosphino)ferrocene]dichloropalladium(II) dichloromethane adduct (0.56 g), cesium carbonate (3.6 g), water (4 mL) and THF (20 mL) was refluxed for 12 hr. Water and ethyl acetate were added to the mixture, and the mixture was passed through the Celite filter. The organic layer was separated, washed with water, brine, dried over Na₂SO₄ and concentrated in vacuo. The residue was purified by chromatography on silica gel (ethyl acetate). The resulting crystals were washed with diisopropyl ether. The title compound was obtained as crystals (0.71 g).

¹H-NMR (300 MHz, DMSO-d₆) δ: 2.14 (s, 3H) 4.64 (s, 2H) 6.50 (d, J=1.9 Hz, 1H) 6.56-6.60 (m, 1H) 7.25-7.42 (m, 5H) 7.63 (s, 1H) 9.71 (s, 1H) 10.63 (s, 1H)

### Preparation 113

### 3-(4-Fluorophenyl)-2-(8-methyl-3-oxo-3,4-dihydro-2H-1,4-benzoxazin-6-yl)acrylaldehyde

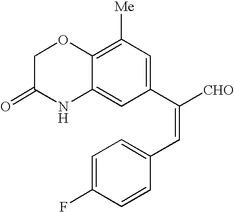

A mixture of 8-methyl-6-(4,4,5,5-tetramethyl-1,3,2-dioxaborolan-2-yl)-2H-1,4-benzoxazin-3(4H)-one (1.0 g), 3-(4-fluorophenyl)-2-iodoacrylaldehyde (1.2 g), [1,1-bis(diphenylphosphino)ferrocene]dichloropalladium(II) dichloromethane adduct (0.56 g), cesium carbonate (3.6 g), water (4 mL) and THF (20 mL) was refluxed for 12 hr. Water and ethyl acetate were added to the mixture, and the mixture was passed through the Celite filter. The organic layer was separated, washed with water, brine, dried over Na₂SO₄ and concentrated in vacuo. The residue was crystallized from ethyl acetate to give the title compound as crystals (0.46 g).

¹H-NMR (300 MHz, DMSO-d₆) δ: 2.15 (s, 3H) 4.64 (s, 2H) 6.47-6.52 (m, 1H) 6.55-6.60 (m, 1H) 7.14-7.25 (m, 2H) 7.31-7.40 (m, 2H) 7.63 (s, 1H) 9.69 (s, 1H) 10.62 (s, 1H).

### Preparation 114

### Methyl 6-amino-6-oxohexanoate

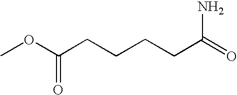

A solution of methyl 6-chloro-6-oxohexanoate (10.0 g) in THF (100 mL) was added dropwise to 28% aqueous ammonia solution (100 mL) under ice cooling, and then the mixture was allowed to warm to room temperature. After stirring, the mixture for 0.5 hr at room temperature, the mixture was concentrated in vacuo. The residue was diluted with THF and the mixture was filtered. The filtrate was dried over Na₂SO₄ and concentrated in vacuo to give the title compound as an oil (8.11 g).

¹H-NMR (300 MHz, DMSO-d₆) δ: 1.40-1.59 (m, 4H) 2.03 (t, J=6.6 Hz, 2H) 2.29 (t, J=6.6 Hz, 2H) 3.58 (s, 3H) 6.68 (s, 1H) 7.21 (s, 1H).

### Preparation 115

### Methyl 4-amino-4-oxobutanoate

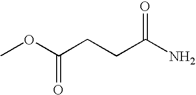

According to a method similar to the procedure for methyl 6-amino-6-oxohexanoate, the title compound was obtained as crystals (5.5 g) from methyl 4-chloro-4-oxobutanoate (11.8 g).

¹H-NMR (300 MHz, CDCl₃) δ: 2.53 (t, J=6.6 Hz, 2H), 2.68 (t, J=6.6 Hz, 2H), 3.70 (s, 3H), 5.59-6.16 (m, 2H).

### Preparation 116

### Methyl 6-amino-6-thioxohexanoate

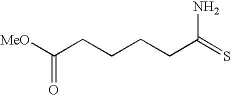

A mixture of methyl 6-amino-6-oxohexanoate (8.0 g), phosphorus pentasulfide (11.2 g) and THF (110 mL) was stirred at room temperature for 72 hr. The mixture was filtered, and the filtrate was concentrated in vacuo. The residue was purified by chromatography on silica gel (hexane→hexane:ethyl acetate=2:3) and followed by crystallization from hexane to give the title compound as crystals (4.88 g).

¹H-NMR (300 MHz, CDCl₃) δ: 1.62-1.90 (m, 4H), 2.37 (t, J=7.0 Hz, 2H), 2.69 (t, J=7.2 Hz, 2H), 3.68 (s, 3H), 7.20 (s, 1H), 7.62 (s, 1H).

### Preparation 117

### Methyl 4-amino-4-thioxobutanoate

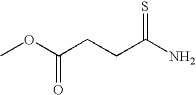

A mixture of methyl 4-amino-4-oxobutanoate (5.4 g), phosphorus pentasulfide (9.2 g) and THF (100 mL) was stirred at room temperature for 48 hr, and the mixture was concentrated in vacuo. The residue was diluted with ethyl acetate, and then water was added. The aqueous layer of the mixture was neutralized with aqueous sodium hydroxide, and then the organic layer was separated. The organic layer was washed with brine, dried over Na₂SO₄ and concentrated in vacuo. The residue was purified by chromatography on silica gel (hexane→hexane:ethyl acetate=1:1) to give the title compound as crystals (1.1 g).

¹H-NMR (300 MHz, CDCl₃) δ: 2.80-2.98 (m, 4H), 3.71 (s, 3H), 7.17-7.91 (m, 2H).

### Preparation 118

### 6-Hydroxyhexanethioamide

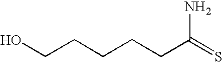

To a solution of methyl 6-amino-6-thioxohexanoate (1.0 g) in ethanol (20 mL) was added sodium tetrahydroborate (1.1 g) under ice cooling, and the mixture was allowed to warm to room temperature. The mixture was stirred for 12 hr, and calcium chloride (1.6 g) was added to the mixture at room temperature. The mixture was stirred for 0.5 hr at room temperature, and then sodium tetrahydroborate (1.1 g) was added to the mixture under ice cooling. Then, the mixture was allowed to warm to room temperature, and stirred for 12 hr. Water was added to the mixture, and the aqueous layer was acidified with 10% hydrochloric acid. The mixture was extracted with ethyl acetate, and the organic layer was dried over Na₂SO₄ and concentrated in vacuo to give the title compound as crystals (0.4 g).

¹H-NMR (300 MHz, DMSO-d₆) δ: 1.18-1.72 (m, 6H), 2.45 (t, J=7.4 Hz, 2H), 3.37 (t, J=6.3 Hz, 2H), 9.10 (s, 1H), 9.29 (s, 1H), 1H unconfirmed.

### Example 156

### 6-[2-(4-Bromophenyl)-2H-thiochromen-3-yl]-2H-1,4-benzoxazin-3(4H)-one

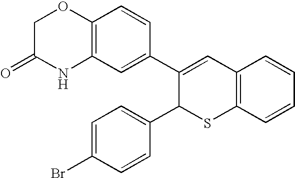

A mixture of 6-[bromo(4 bromophenyl)acetyl]-2H-1,4-benzoxazin-3(4H)-one (0.43 g), 2-mercaptobenzyltriphenylphosphonium bromide (0.51 g) and THF (4 mL) was stirred at 60° C. for 3 hr under nitrogen atmosphere, and then potassium tert-butoxide (0.25 g) was added to the mixture. The mixture was allowed to warm to 80° C., and stirred for 4 hr under nitrogen atmosphere. Water and 10% hydrochloric acid were added to the mixture, and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:1) and followed by crystallization from methanol to give the title compound as crystals (0.27 g).

mp. 184-187° C.

¹H-NMR (300 MHz, DMSO-d₆) δ: 4.57 (s, 2H), 5.33 (s, 1H), 6.94 (d, J=8.5 Hz, 1H), 7.03 (d, J=2.1 Hz, 1H), 7.07-7.29 (m, 7H), 7.39-7.48 (m, 3H), 10.72 (s, 1H).

### Example 157

### 6-[2-(3-Bromophenyl)-2H-thiochromen-3-yl]-2H-1,4-benzoxazin-3(4H)-one

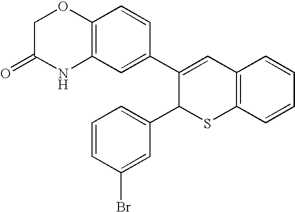

The title compound was obtained as crystals (2.04 g) from 6-[bromo(3-bromophenyl)acetyl]-2H-1,4-benzoxazin-3(4H)-one (3.00 g) according to a method similar to the procedure for 6-[2-(4-bromophenyl)-2H-thiochromen-3-yl]-2H-1,4-benzoxazin-3(4H)-one.

mp. 232-234° C. (methanol).

¹H-NMR (300 MHz, DMSO-d₆) δ: 4.57 (s, 2H), 5.37 (s, 1H), 6.95 (d, J=8.7 Hz, 1H), 7.05 (d, J=2.3 Hz, 1H), 7.08-7.27 (m, 6H), 7.29 (s, 1H), 7.34-7.41 (m, 1H), 7.43-7.52 (m, 2H), 10.73 (s, 1H).

### Example 158

### 6-[2-(4-Fluorophenyl)-2H-thiochromen-3-yl]-2H-1,4-benzoxazin-3(4H)-one

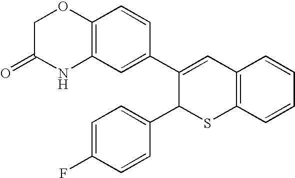

A mixture of 6-[bromo(4-fluorophenyl)acetyl]-2H-1,4-benzoxazin-3(4H)-one (0.37 g), 2-mercaptobenzyltriphenylphosphonium bromide (0.51 g) and THF (4 mL) was stirred at 60° C. for 3 hr under nitrogen atmosphere, and then potassium tert-butoxide (0.25 g) was added to the mixture. The mixture was allowed to warm to 80° C., and stirred for 4 hr under nitrogen atmosphere. Water and 10% hydrochloric acid were added to the mixture, and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:1) and followed by crystallization from methanol to give the title compound as crystals (0.23 g).

mp. 188-190° C.

¹H-NMR (300 MHz, DMSO-d₆) δ: 4.57 (s, 2H), 5.34 (s, 1H), 6.93 (d, J=8.3 Hz, 1H), 7.00-7.34 (m, 10H), 7.41-7.48 (m, 1H), 10.73 (s, 1H).

### Example 159

### 6-[2-(4-Nitrophenyl)-2H-thiochromen-3-yl]-2H-1,4-benzoxazin-3(4H)-one

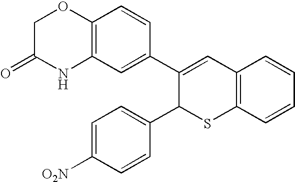

The title compound was obtained as crystals (0.13 g) from 6-[bromo(4-nitrophenyl)acetyl]-2H-1,4-benzoxazin-3(4H)-one (0.39 g) according to a method similar to the procedure for 6-[2-(4-bromophenyl)-2H-thiochromen-3-yl]-2H-1,4-benzoxazin-3(4H)-one.

mp. 139-144° C. (dichloromethane/diethyl ether).

¹H-NMR (300 MHz, DMSO-d₆) δ: 4.57 (s, 2H), 5.54 (s, 1H), 6.95 (d, J=8.3 Hz, 1H), 7.02 (d, J=1.9 Hz, 1H), 7.08-7.25 (m, 4H), 7.33 (s, 1H), 7.43-7.58 (m, 3H), 8.12 (d, J=8.7 Hz, 2H), 10.71 (s, 1H).

### Example 160

### 6-[2-(3-Nitrophenyl)-2H-thiochromen-3-yl]-2H-1,4-benzoxazin-3(4H)-one

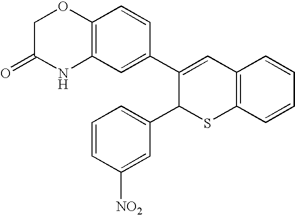

The title compound was obtained as crystals (1.24 g) from 6-[bromo(3-nitrophenyl)acetyl]-2H-1,4-benzoxazin-3(4H)-one (3.00 g) according to a method similar to the procedure for 6-[2-(4-bromophenyl)-2H-thiochromen-3-yl]-2H-1,4-benzoxazin-3(4H)-one.

mp. 214-216° C. (ethyl acetate).

¹H-NMR (300 MHz, DMSO-d₆) δ: 4.57 (s, 2H), 5.59 (s, 1H), 6.95 (d, J=8.7 Hz, 1H), 7.03 (d, J=1.9 Hz, 1H), 7.10-7.26 (m, 4H), 7.36 (s, 1H), 7.46-7.59 (m, 2H), 7.68 (d, J=8.0 Hz, 1H), 8.01-8.10 (m, 1H), 8.20 (t, J=1.7 Hz, 1H), 10.72 (s, 1H).

### Example 161

### 6-[6-Fluoro-2-(4-fluorophenyl)-2H-thiochromen-3-yl]-2H-1,4-benzoxazin-3(4H)-one

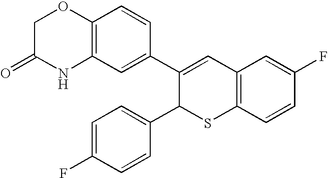

According to a method similar to the procedure for 6-[2-(4-bromophenyl)-2H-thiochromen-3-yl]-2H-1,4-benzoxazin-3(4H)-one, 6-[bromo(4-fluorophenyl)acetyl]-2H-1,4-benzoxazin-3(4H)-one (0.37 g) was coupled with (5-fluoro-2-mercaptobenzyl)(triphenyl)phosphonium bromide (0.53 g) to give the title compound as crystals (0.16 g).

mp. 191-192° C. (methanol).

¹H-NMR (300 MHz, DMSO-d₆) δ: 4.57 (s, 2H), 5.37 (s, 1H), 6.88-7.32 (m, 10H), 7.37 (dd, J=9.8, 2.7 Hz, 1H), 10.76 (s, 1H).

### Example 162

### 6-[2-(4-Aminophenyl)-2H-thiochromen-3-yl]-2H-1,4-benzoxazin-3(4H)-one

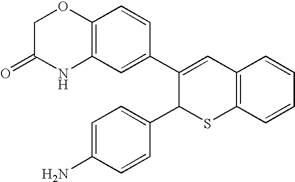

To a mixture of 6-[2-(4-nitrophenyl)-2H-thiochromen-3-yl]-2H-1,4-benzoxazin-3(4H)-one (1.15 g), acetic acid (12 mL) and THF (24 mL) was added zinc (powder, 2.7 g) at 45° C., and the mixture was stirred for 0.5 hr. The mixture was filtered, and then the filtrate was concentrated in vacuo. Saturated aqueous sodium bicarbonate solution was added to the residue, and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:2). The resulting crystals were washed with diisopropyl ether to give the title compound as crystals (0.76 g).

mp. 186-189° C.

¹H-NMR (300 MHz, DMSO-d₆) δ: 4.56 (s, 2H), 5.02 (s, 2H), 5.06 (s, 1H), 6.30-6.42 (m, 2H), 6.83-6.96 (m, 3H), 7.01-7.22 (m, 6H), 7.36-7.46 (m, 1H), 10.72 (s, 1H).

### Example 163

### 6-[2-(3-Aminophenyl)-2H-thiochromen-3-yl]-2H-1,4-benzoxazin-3(4H)-one

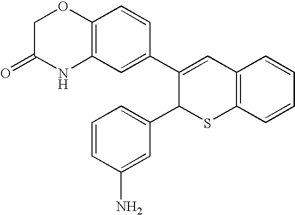

To a mixture of 6-[2-(3-nitrophenyl)-2H-thiochromen-3-yl]-2H-1,4-benzoxazin-3(4H)-one (1.0 g), acetic acid (10 mL) and THF (20 mL) was added zinc (powder, 2.7 g) at 45° C., and the mixture was stirred for 0.5 hr. The mixture was filtered, and then the filtrate was concentrated in vacuo. Saturated aqueous sodium bicarbonate solution was added to the residue, and the mixture was extracted with ethyl acetate. The organic layer was washed with water and brine, dried over Na₂SO₄ and concentrated in vacuo. The resulting crystals were washed with ethyl acetate to give the title compound as crystals (0.84 g).

mp. 238-243° C.

¹H-NMR (300 MHz, DMSO-d₆) δ: 4.56 (s, 2H), 5.00 (s, 2H), 5.09 (s, 1H), 6.29-6.49 (m, 3H), 6.83 (t, J=7.8 Hz, 1H), 6.92 (d, J=8.3 Hz, 1H), 7.03-7.22 (m, 6H), 7.36-7.45 (m, 1H), 10.74 (s, 1H).

### Example 164

### 6-(6-Fluoro-2-propyl-2H-thiochromen-3-yl)-2H-1,4-benzoxazin-3(4H)-one

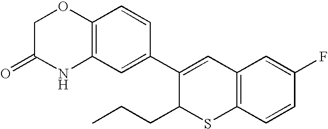

According to a method similar to the procedure for 6-[2-(4-bromophenyl)-2H-thiochromen-3-yl]-2H-1,4-benzoxazin-3(4H)-one, 6-(2-bromopentanoyl)-2H-1,4-benzoxazin-3(4H)-one (0.31 g) was coupled with (5-fluoro-2-mercaptobenzyl)(triphenyl)phosphonium bromide (0.53 g) to give the title compound as crystals (0.14 g).

mp. 161-162° C. (methanol).

¹H-NMR (300 MHz, DMSO-d₆) δ: 0.81 (t, J=6.4 Hz, 3H), 1.21-1.58 (m, 4H), 3.89 (t, J=6.6 Hz, 1H), 4.62 (s, 2H), 6.88 (s, 1H), 6.97-7.09 (m, 2H), 7.13 (d, J=1.9 Hz, 1H), 7.20-7.30 (m, 2H), 7.34 (dd, J=8.7, 5.7 Hz, 1H), 10.78 (s, 1H).

### Example 165

### 6-(6-Fluoro-2H-thiochromen-3-yl)-2H-1,4-benzoxazin-3(4H)-one

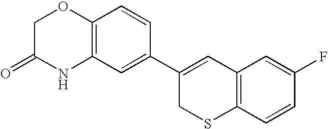

According to a method similar to the procedure for 6-[2-(4-bromophenyl)-2H-thiochromen-3-yl]-2H-1,4-benzoxazin-3(4H)-one, 6-(chloroacetyl)-2H-1,4-benzoxazin-3(4H)-one (0.3 g) was coupled with (5-fluoro-2-mercaptobenzyl)(triphenyl)phosphonium bromide (0.71 g) to give the title compound as crystals (0.16 g).

mp. 229-234° C. (methanol).

¹H-NMR (300 MHz, DMSO-d₆) δ: 3.84 (s, 2H), 4.62 (s, 2H), 6.85 (s, 1H), 6.96-7.06 (m, 2H), 7.09 (d, J=2.3 Hz, 1H), 7.16-7.24 (m, 2H), 7.30 (dd, J=8.7, 5.7 Hz, 1H), 10.78 (s, 1H).

### Example 166

### 6-[7-(3-Bromophenyl)-7H-[1,2,4]triazolo[3,4-b][1,3,4]thiadiazin-6-yl]-2H-1,4-benzoxazin-3(4H)-one

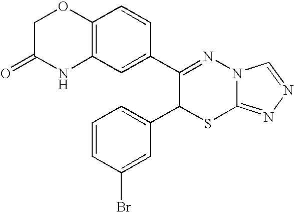

The title compound was obtained as crystals (3.0 g) from 6-[bromo(3-bromophenyl)acetyl]-2H-1,4-benzoxazin-3(4H)-one (3.0 g) according to a method similar to the procedure for 6-[7-(4-chlorophenyl)-7H-[1,2,4]triazolo[3,4-b][1,3,4]thiadiazin-6-yl]-2H-1,4-benzoxazin-3(4H)-one.

mp. 234-236° C. (ethyl acetate).

¹H-NMR (300 MHz, DMSO-d₆) δ: 4.68 (s, 2H), 6.35 (s, 1H), 7.03 (d, J=8.0 Hz, 1H), 7.10 (d, J=8.7 Hz, 1H), 7.27 (t, J=8.0 Hz, 1H), 7.42-7.56 (m, 3H), 7.57 (d, J=2.3 Hz, 1H), 9.30 (s, 1H), 10.96 (s, 1H).

### Example 167

### 6-[7-(2-Bromophenyl)-7H-[1,2,4]triazolo[3,4-b][1,3,4]thiadiazin-6-yl]-2H-1,4-benzoxazin-3(4H)-one

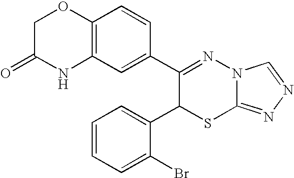

The title compound was obtained as crystals (4.37 g) from 6-[bromo(2-bromophenyl)acetyl]-2H-1,4-benzoxazin-3(4H)-one (5.0 g) according to a method similar to the procedure for 6-[7-(4-chlorophenyl)-7H-[1,2,4]triazolo[3,4-b][1,3,4]thiadiazin-6-yl]-2H-1,4-benzoxazin-3(4H)-one.

mp. 249-251° C. (methanol).

¹H-NMR (300 MHz, DMSO-d₆) δ: 4.66 (s, 2H), 6.15 (s, 1H), 6.74 (dd, J=7.2, 1.9 Hz, 1H), 7.08 (d, J=8.7 Hz, 1H), 7.20-7.39 (m, 3H), 7.49 (d, J=1.9 Hz, 1H), 7.83 (dd, J=7.6, 1.5 Hz, 1H), 9.31 (s, 1H), 10.97 (s, 1H).

### Example 168

### 6-[7-(2-Methylphenyl)-7H-[1,2,4]triazolo[3,4-b][1,3,4]thiadiazin-6-yl]-2H-1,4-benzoxazin-3(4H)-one

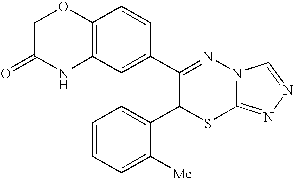

The title compound was obtained as crystals (0.33 g) from 6-[bromo(2-methylphenyl)acetyl]-2H-1,4-benzoxazin-3(4H)-one (0.5 g) according to a method similar to the procedure for 6-[7-(4-chlorophenyl)-7H-[1,2,4]triazolo[3,4-b][1,3,4]thiadiazin-6-yl]-2H-1,4-benzoxazin-3(4H)-one.

mp. 226-228° C. (AcOEt).

¹H-NMR (300 MHz, DMSO-d₆) δ: 2.58 (s, 3H), 4.66 (s, 2H), 6.21 (s, 1H), 6.52 (d, J=7.4 Hz, 1H), 6.96-7.10 (m, 2H), 7.17-7.26 (m, 1H), 7.35 (d, J=7.4 Hz, 1H), 7.40-7.50 (m, 2H), 9.30 (s, 1H), 10.95 (s, 1H).

### Example 169

### 3-[3-(3-Oxo-3,4-dihydro-2H-1,4-benzoxazin-6-yl)-2H-thiochromen-2-yl]benzonitrile

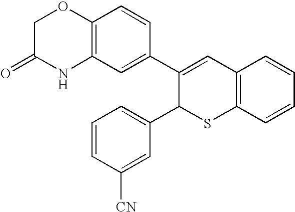

A mixture of 6-[2-(3-bromophenyl)-2H-thiochromen-3-yl]-2H-1,4-benzoxazin-3(4H)-one (0.4 g), zinc cyanide (58 mg), tetrakis(triphenylphosphine)palladium(0) (52 mg) and 1-methyl-2-pyrrolidone (4 mL) was stirred at 100° C. for 12 hr under argon atmosphere, and then concentrated in vacuo. The residue was diluted with ethyl acetate and the mixture was filtered. The filtrate was washed with water and brine, dried over Na₂SO₄ and concentrated in vacuo. The residue was purified by chromatography on silica gel (hexane→hexane:ethyl acetate=1:1) and followed by crystallization from methanol to give the title compound as crystals (210 mg).

mp. 213-215° C.

¹H-NMR (300 MHz, DMSO-d₆) δ: 4.57 (s, 2H), 5.44 (s, 1H), 6.95 (d, J=8.5 Hz, 1H), 7.02 (d, J=2.3 Hz, 1H), 7.10-7.24 (m, 4H), 7.33 (s, 1H), 7.41-7.51 (m, 2H), 7.52-7.58 (m, 1H), 7.64-7.69 (m, 1H), 7.70-7.73 (m, 1H), 10.72 (s, 1H).

### Example 170

### 6-[2-(2-Fluorophenyl)-2H-imidazo[2,1-b][1,3,4]thiadiazin-3-yl]-2H-1,4-benzoxazin-3(4H)-one

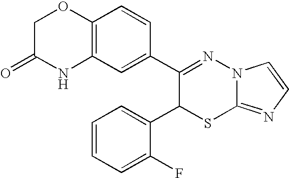

A mixture of 6-[bromo(2-fluorophenyl)acetyl]-2H-1,4-benzoxazin-3(4H)-one (130 mg), 1-amino-1H-imidazole-2-thiol (50 mg), ethanol (2 mL) and toluene (1 mL) was refluxed for 24 hr and then concentrated in vacuo. Water and saturated aqueous sodium bicarbonate solution were added to the residue, and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:2) and followed by crystallization from methanol to give the title compound as crystals (53 mg).

mp. 210-212° C.

¹H-NMR (300 MHz, DMSO-d₆) δ: 4.65 (s, 2H), 6.22 (s, 1H), 6.65-6.75 (m, 1H), 7.00-7.09 (m, 3H), 7.27-7.48 (m, 3H), 7.52 (d, J=2.1 Hz, 1H), 7.81 (d, J=1.5 Hz, 1H), 10.87 (s, 1H).

### Example 171

### 6-[2-(3-Fluorophenyl)-2H-imidazo[2,1-b][1,3,4]thiadiazin-3-yl]-2H-1,4-benzoxazin-3(4H)-one

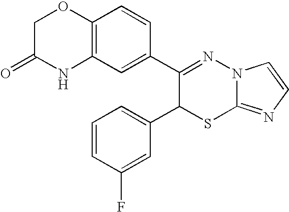

The title compound was obtained as crystals (74 mg) from 6-[bromo(3-fluorophenyl)acetyl]-2H-1,4-benzoxazin-3(4H)-one (130 mg) according to a method similar to the procedure for 6-[2-(2-Fluorophenyl)-2H-imidazo[2,1-b][1,3,4]thiadiazin-3-yl]-2H-1,4-benzoxazin-3(4H)-one

mp. 196-198° C. (methanol).

¹H-NMR (300 MHz, DMSO-d₆) δ: 4.66 (s, 2H), 6.17 (s, 1H), 6.91 (d, J=8.0 Hz, 1H), 6.96-7.20 (m, 4H), 7.30-7.48 (m, 2H), 7.56 (d, J=1.9 Hz, 1H), 7.80 (d, J=1.5 Hz, 1H), 10.90 (s, 1H).

### Example 172

### 7-(2-Amino-6-phenyl-6H-1,3-thiazin-5-yl)-1H-pyrido[2,3-b][1,4]oxazin-2(3H)-one

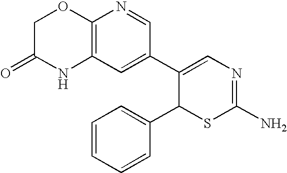

A mixture of 2-(2-oxo-2,3-dihydro-1H-pyrido[2,3-b][1,4]oxazin-7-yl)-3-phenylacrylaldehyde (0.2 g), thiourea (70 mg), 10% hydrochloric acid (0.4 mL) and dioxane (4 mL) was stirred at 80° C. for 12 hr, and then concentrated in vacuo. The residue was treated with aqueous sodium bicarbonate solution, and the resulting crystals were collected. The title compound was obtained as crystals (0.24 g).

¹H-NMR (300 MHz, DMSO-d₆) δ: 4.71 (s, 2H), 5.27 (s, 1H), 7.02 (s, 2H), 7.16 (d, J=2.3 Hz, 1H), 7.19-7.35 (m, 6H), 7.78 (d, J=2.3 Hz, 1H), 10.75 (s, 1H).

### Example 173

### 7-(7-Phenyl-7H-imidazo[2,1-b][1,3]thiazin-6-yl)-1H-pyrido[2,3-b][1,4]oxazin-2(3H)-one

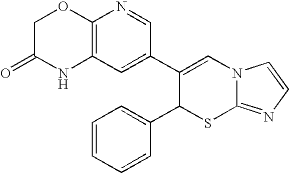

A mixture of 7-(2-amino-6-phenyl-6H-1,3-thiazin-5-yl)-1H-pyrido[2,3-b][1,4]oxazin-2(3H)-one (0.1 g), 45% chloroacetaldehyde in water (0.26 g), ethanol (1 mL) and 1,2-dimethoxyethane (5 mL) was refluxed for 12 hr, and then concentrated in vacuo. Aqueous sodium bicarbonate solution was added to the residue, and the mixture was extracted with ethyl acetate. The organic layer was washed brine, dried over Na₂SO₄ and concentrated in vacuo. The residue was purified by chromatography on silica gel (ethyl acetate→ethyl acetate:THF=3:1) and followed by crystallization from methanol/diisopropyl ether to give the title compound as crystals (29 mg).

mp. 246-249° C. (decomp.).

¹H-NMR (300 MHz, DMSO-d₆) δ: 4.77 (s, 2H), 5.60 (s, 1H), 6.97 (d, J=1.5 Hz, 1H), 7.15-7.35 (m, 6H), 7.58 (d, J=1.5 Hz, 1H), 7.91 (s, 1H), 7.96 (d, J=2.3 Hz, 1H), 10.91 (s, 1H).

### Example 174

### 6-[2-(2-Fluorophenyl)-7-methyl-2H-imidazo[2,1-b][1,3,4]thiadiazin-3-yl]-2H-1,4-benzoxazin-3(4H)-one

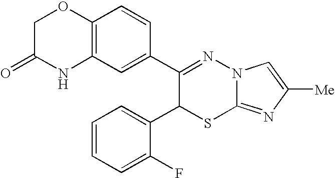

A mixture of 6-[bromo(2-fluorophenyl)acetyl]-2H-1,4-benzoxazin-3(4H)-one (0.47 g), 1-amino-4-methyl-1H-imidazole-2-thiol (0.2 g), ethanol (6 mL) and toluene (3 mL) was refluxed for 36 hr and then concentrated in vacuo. Water and saturated aqueous sodium bicarbonate solution were added to the residue, and the mixture was extracted with ethyl acetate. The organic layer was washed with water and brine, dried over Na₂SO₄ and concentrated in vacuo. The residue was purified by chromatography on silica gel (hexane→hexane:ethyl acetate=3:2) and followed by crystallization from methanol/diisopropyl ether to give the title compound as crystals (0.36 g).

mp. 148-152° C.

¹H-NMR (300 MHz, DMSO-d₆) δ: 2.08 (d, J=0.8 Hz, 3H), 4.64 (s, 2H), 6.16 (s, 1H), 6.66-6.76 (m, 1H), 6.99-7.11 (m, 2H), 7.27-7.44 (m, 3H), 7.47-7.54 (m, 2H), 10.85 (s, 1H).

### Example 175

### 6-[2-(3-Fluorophenyl)-7-methyl-2H-imidazo[2,1-b][1,3,4]thiadiazin-3-yl]-2H-1,4-benzoxazin-3(4H)-one

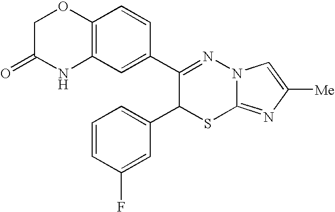

The title compound was obtained as crystals (157 mg) from 6-[bromo(3-fluorophenyl)acetyl]-2H-1,4-benzoxazin-3(4H)-one (240 mg) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 221-223° C. (decomp., ethyl acetate/hexane).

¹H-NMR (300 MHz, DMSO-d₆) δ: 2.08 (d, J=1.1 Hz, 3H), 4.65 (s, 2H), 6.12 (s, 1H), 6.88-6.95 (m, 1H), 6.98-7.09 (m, 2H), 7.10-7.19 (m, 1H), 7.31-7.44 (m, 2H), 7.50 (d, J=1.1 Hz, 1H), 7.54 (d, J=2.1 Hz, 1H), 10.88 (s, 1H).

### Example 176

### 6-[2-(4-Fluorophenyl)-7-methyl-2H-imidazo[2,1-b][1,3,4]thiadiazin-3-yl]-2H-1,4-benzoxazin-3(4H)-one

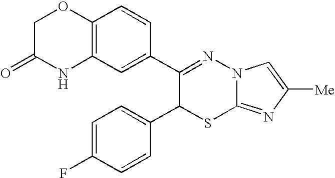

The title compound was obtained as crystals (215 mg) from 6-[bromo(4-fluorophenyl)acetyl]-2H-1,4-benzoxazin-3(4H)-one (240 mg) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 240° C. (decomp., ethyl acetate/hexane).

¹H-NMR (300 MHz, DMSO-d₆) δ: 2.08 (s, 3H), 4.65 (s, 2H), 6.10 (s, 1H), 7.05 (d, J=8.6 Hz, 1H), 7.11-7.25 (m, 4H), 7.39 (dd, J=8.6, 2.0 Hz, 1H), 7.47 (s, 1H), 7.54 (d, J=2.0 Hz, 1H), 10.87 (s, 1H).

### Example 177

### 6-[2-(2-Chlorophenyl)-7-methyl-2H-imidazo[2,1-b][1,3,4]thiadiazin-3-yl]-2H-1,4-benzoxazin-3(4H)-one

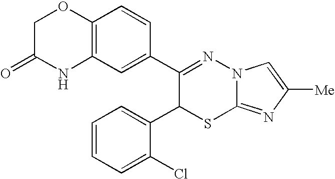

The title compound was obtained as crystals (280 mg) from 6-[bromo(2-chlorophenyl)acetyl]-2H-1,4-benzoxazin-3(4H)-one (490 mg) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 222-223° C. (decomp., methanol).

¹H-NMR (300 MHz, DMSO-d₆) δ: 2.08 (d, J=1.0 Hz, 3H), 4.64 (s, 2H), 5.99 (s, 1H), 6.70 (dd, J=8.0, 1.5 Hz, 1H), 7.03 (d, J=8.3 Hz, 1H), 7.16-7.26 (m, 1H), 7.27-7.40 (m, 2H), 7.46 (d, J=1.9 Hz, 1H), 7.53 (d, J=1.0 Hz, 1H), 7.64 (dd, J=8.0, 1.1 Hz, 1H), 10.88 (s, 1H).

### Example 178

### 6-[2-(3-Chlorophenyl)-7-methyl-2H-imidazo[2,1-b][1,3,4]thiadiazin-3-yl]-2H-1,4-benzoxazin-3(4H)-one

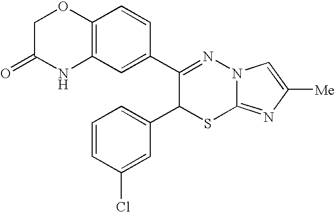

The title compound was obtained as crystals (236 mg) from 6-[bromo(3-chlorophenyl)acetyl]-2H-1,4-benzoxazin-3(4H)-one (250 mg) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 191-193° C. (ethyl acetate/hexane).

¹H-NMR (300 MHz, DMSO-d₆) δ: 2.08 (s, 3H), 4.65 (s, 2H), 6.12 (s, 1H), 6.98-7.10 (m, 2H), 7.25-7.44 (m, 4H), 7.48-7.57 (m, 2H), 10.87 (s, 1H).

### Example 179

### 6-[2-(4-Chlorophenyl)-7-methyl-2H-imidazo[2,1-b][1,3,4]thiadiazin-3-yl]-2H-1,4-benzoxazin-3(4H)-one

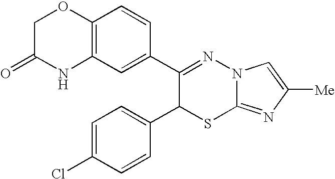

The title compound was obtained as crystals (168 mg) from 6-[bromo(4-chlorophenyl)acetyl]-2H-1,4-benzoxazin-3(4H)-one (250 mg) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 155-160° C. (ethyl acetate/hexane).

¹H-NMR (300 MHz, DMSO-d₆) δ: 2.07 (d, J=1.1 Hz, 3H), 4.65 (s, 2H), 6.11 (s, 1H), 7.05 (d, J=8.3 Hz, 1H), 7.13-7.21 (m, 2H), 7.35-7.44 (m, 3H), 7.47 (d, J=1.1 Hz, 1H), 7.53 (d, J=2.3 Hz, 1H), 10.87 (s, 1H).

### Example 180

### 6-[7-Methyl-2-(1,3-thiazol-2-yl)-2H-imidazo[2,1-b][1,3,4]thiadiazin-3-yl]-2H-1,4-benzoxazin-3(4H)-one

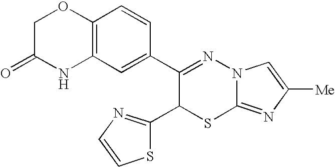

The title compound was obtained as crystals (20.8 mg) from 6-[bromo(1,3-thiazol-2-yl)acetyl]-2H-1,4-benzoxazin-3(4H)-one (150 mg) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 188-190° C. (decomp., ethyl acetate/hexane).

¹H-NMR (300 MHz, DMSO-d₆) δ: 2.09 (s, 3H) 4.66 (s, 2H) 6.51 (s, 1H) 7.08 (d, J=8.3 Hz, 1H) 7.42 (d, J=0.8 Hz, 1H) 7.50 (dd, J=8.3, 2.3 Hz, 1H) 7.57 (d, J=2.3 Hz, 1H) 7.64 (d, J=3.0 Hz, 1H) 7.71 (d, J=3.0 Hz, 1H) 10.88 (s, 1H)

### Example 181

### 6-(2-Oxo-6-phenyl-3,6-dihydro-2H-1,3-thiazin-5-yl)-2H-1,4-benzoxazin-3(4H)-one

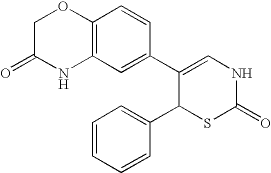

To a solution of 6-(2-amino-6-phenyl-6H-1,3-thiazin-5-yl)-2H-1,4-benzoxazin-3(4H)-one (100 mg) in DMF (1 mL) was added 3-methylbutyl nitrite (70 mg) dropwise at 65° C., and the mixture was stirred for 6 hr. Water was added to the mixture and the mixture was extracted with ethyl acetate. The organic layer was washed with water and brine, dried over Na₂SO₄ and concentrated in vacuo. The residue was purified by chromatography on silica gel (hexane→hexane:ethyl acetate=1:2) and followed by crystallization from ethyl acetate/diisopropyl ether to give the title compound as crystals (36.8 mg).

mp. 174-176° C.

¹H-NMR (300 MHz, DMSO-d₆) δ: 4.51 (s, 2H), 5.42 (s, 1H), 6.70 (d, J=5.9 Hz, 1H), 6.79-6.94 (m, 3H), 7.20-7.42 (m, 5H), 10.26 (d, J=5.9 Hz, 1H), 10.63 (s, 1H).

### Example 182

### 6-(2-Amino-6-phenyl-6H-1,3-thiazin-5-yl)-8-methyl-2H-1,4-benzoxazin-3(4H)-one

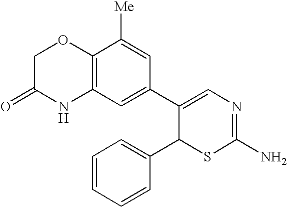

A mixture of 2-(8-methyl-3-oxo-3,4-dihydro-2H-1,4-benzoxazin-6-yl)-3-phenylacrylaldehyde (0.3 g), thiourea (82 mg), 10% hydrochloric acid (0.6 mL) and THF (6 mL) was refluxed for 6 hr, and then concentrated in vacuo. The residue was diluted with water, and then saturated aqueous sodium bicarbonate solution was added to the mixture. The resulting crystals were collected by filtration, and suspended in ethyl acetate. The mixture was refluxed for 10 min., and then cooled to room temperature. The resulting crystals were collected by filtration. The title compound was obtained as crystals (0.31 g).

mp. 206-208° C.

¹H-NMR (300 MHz, DMSO-d₆) δ: 2.09 (s, 3H), 4.51 (s, 2H), 5.17 (s, 1H), 6.72 (s, 1H), 6.76-6.90 (m, 3H), 7.13-7.34 (m, 6H), 10.53 (s, 1H).

### Example 183

### 6-[2-Amino-6-(4-fluorophenyl)-6H-1,3-thiazin-5-yl]-8-methyl-2H-1,4-benzoxazin-3(4H)-one

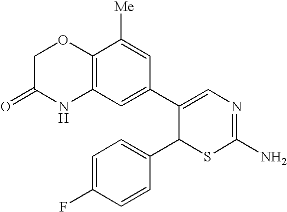

The title compound was obtained as crystals (0.28 g) from 3-(4-fluorophenyl)-2-(8-methyl-3-oxo-3,4-dihydro-2H-1,4-benzoxazin-6-yl)acrylaldehyde (0.3 g) according to a method similar to the procedure for 6-(2-Amino-6-phenyl-6H-1,3-thiazin-5-yl)-8-methyl-2H-1,4-benzoxazin-3(4H)-one.

mp. 213-214° C. (ethyl acetate).

¹H-NMR (300 MHz, DMSO-d₆) δ: 2.10 (s, 3H), 4.51 (s, 2H), 5.21 (s, 1H), 6.70 (d, J=1.9 Hz, 1H), 6.78-6.92 (m, 3H), 7.05-7.19 (m, 3H), 7.23-7.34 (m, 2H), 10.53 (s, 1H).

### Example 184

### 6-[7-(4-Fluorophenyl)-7H-imidazo[2,1-b][1,3]thiazin-6-yl]-8-methyl-2H-1,4-benzoxazin-3(4H)-one

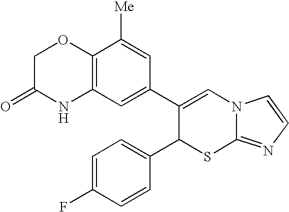

A mixture of 6-[2-amino-6-(4-fluorophenyl)-6H-1,3-thiazin-5-yl]-8-methyl-2H-1,4-benzoxazin-3(4H)-one (0.2 g), 45% chloroacetaldehyde in water (0.47 g), ethanol (1 mL) and 1,2-dimethoxyethane (5 mL) was refluxed for 24 hr, and then concentrated in vacuo. Saturated aqueous sodium bicarbonate solution was added to the residue, and the mixture was extracted with a mixture of THF and ethyl acetate. The organic layer was washed with water and brine, dried over Na₂SO₄ and concentrated in vacuo. The resulting crystals were washed with ethyl acetate and methanol, and then suspended in THF. The mixture was refluxed for 10 min., and then cooled to room temperature. The resulting crystals were collected by filtration. The title compound was obtained as crystals (70 mg).

mp. 285-286° C.

¹H-NMR (300 MHz, DMSO-d₆) δ: 2.15 (s, 3H), 4.57 (s, 2H), 5.54 (s, 1H), 6.73 (d, J=1.9 Hz, 1H), 6.95 (d, J=0.8 Hz, 1H), 7.00 (d, J=1.9 Hz, 1H), 7.07-7.18 (m, 2H), 7.20-7.30 (m, 2H), 7.57 (s, 1H), 7.79 (s, 1H), 10.67 (s, 1H).

### Example 185

### 6-[7-(4-Fluorophenyl)-7H-[1,2,4]triazolo[3,4-b][1,3,4]thiadiazin-6-yl]-8-methyl-2H-1,4-benzoxazin-3(4H)-one

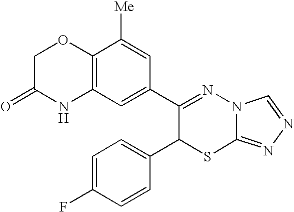

A mixture of 6-[bromo(4-fluorophenyl)acetyl]-8-methyl-2H-1,4-benzoxazin-3(4H)-one (0.5 g), 4-amino-4H-1,2,4-triazole-3-thiol (0.16 g), ethanol (10 mL) and toluene (5 mL) was refluxed for 6 hr and then concentrated in vacuo. Water and saturated aqueous sodium bicarbonate solution were added to the mixture, and the mixture was extracted with a solution of THF and ethyl acetate. The organic layer was washed with water and brine, dried over Na₂SO₄ and concentrated in vacuo. The residue was crystallized from THF/ethyl acetate to give the title compound as crystals (0.46 g).

mp. 170-173° C.

¹H-NMR (300 MHz, DMSO-d₆) δ: 2.19 (s, 3H), 4.69 (s, 2H), 6.32 (s, 1H), 7.12-7.25 (m, 4H), 7.40 (d, J=2.1 Hz, 1H), 7.44 (d, J=2.1 Hz, 1H), 9.24 (s, 1H), 10.87 (s, 1H).

### Example 186

### 6-[2-(4-Fluorophenyl)-7-methyl-2H-imidazo[2,1-b][1,3,4]thiadiazin-3-yl]-8-methyl-2H-1,4-benzoxazin-3(4H)-one

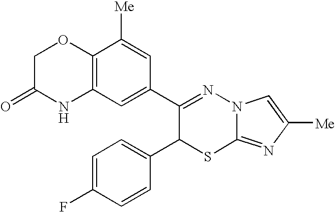

The title compound was obtained as crystals (0.26 g) from 6-[bromo(4-fluorophenyl)acetyl]-8-methyl-2H-1,4-benzoxazin-3(4H)-one (0.26 g) according to a method similar to the procedure for 6-[2-(2-fluorophenyl)-7-methyl-2H-imidazo[2,1-b][1,3,4]thiadiazin-3-yl]-2H-1,4-benzoxazin-3(4H)-one

mp. 144-146° C. (ethyl acetate/hexane).

¹H-NMR (300 MHz, DMSO-d₆) δ: 2.08 (d, J=0.8 Hz, 3H), 2.18 (s, 3H), 4.66 (s, 2H), 6.09 (s, 1H), 7.11-7.24 (m, 4H), 7.37 (s, 2H), 7.47 (d, J=1.1 Hz, 1H), 10.81 (s, 1H).

### Example 187

### 6-[2-(4-Fluorophenyl)-7-(trifluoromethyl)-2H-imidazo[2,1-b][1,3,4]thiadiazin-3-yl]-8-methyl-2H-1,4-benzoxazin-3(4H)-one

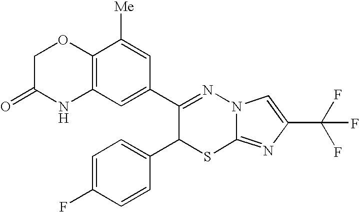

According to a method similar to the procedure for 6-[2-(2-fluorophenyl)-7-methyl-2H-imidazo[2,1-b][1,3,4]thiadiazin-3-yl]-2H-1,4-benzoxazin-3(4H)-one, 6-[bromo(4-fluorophenyl)acetyl]-8-methyl-2H-1,4-benzoxazin-3(4H)-one (0.28 g) was reacted with 1-amino-4-(trifluoromethyl)-1H-imidazole-2-thiol (0.15 g) to give the title compound as crystals (0.31 g).

mp. 136-138° C. (ethyl acetate/diisopropyl ether).

¹H-NMR (300 MHz, DMSO-d₆) δ: 2.19 (s, 3H), 4.69 (s, 2H), 6.28 (s, 1H), 7.15-7.27 (m, 4H), 7.38 (d, J=2.1 Hz, 1H), 7.40-7.44 (m, J=1.7 Hz, 1H), 8.52-8.56 (m, 1H), 10.89 (s, 1H).

### Example 188

### 6-(2-Methyl-6-phenyl-6H-1,3-thiazin-5-yl)-2H-1,4-benzoxazin-3(4H)-one

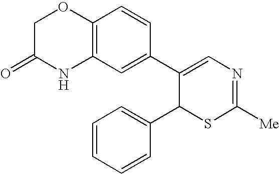

A mixture of 2-(3-oxo-3,4-dihydro-2H-1,4-benzoxazin-6-yl)-3-phenylacrylaldehyde (100 mg), ethanethioamide (30 mg) and 4N-hydrochloric acid in ethyl acetate (2 mL) was stirred at room temperature for 24 hr. Ethanol (2 mL) was added to the mixture, and the mixture was refluxed for 4 hr. The mixture was concentrated in vacuo, and then saturated aqueous sodium bicarbonate solution and water were added to the residue. The mixture was extracted with ethyl acetate, and the organic layer was washed with water and brine, dried over Na₂SO₄ and concentrated in vacuo. The residue was purified by chromatography on silica gel (hexane→hexane:ethyl acetate=1:1) and followed by crystallization from ethyl acetate/hexane to give the title compound as crystals (47 mg).

mp. 187-189° C.

¹H-NMR (300 MHz, DMSO-d₆) δ: 2.16 (s, 3H), 4.54 (s, 2H), 5.36 (s, 1H), 6.89 (d, J=8.7 Hz, 1H), 6.99 (d, J=2.3 Hz, 1H), 7.05 (dd, J=8.7, 2.3 Hz, 1H), 7.17-7.35 (m, 5H), 7.46 (s, 1H), 10.67 (s, 1H).

### Example 189

### 6-(2,6-Diphenyl-6H-1,3-thiazin-5-yl)-2H-1,4-benzoxazin-3(4H)-one

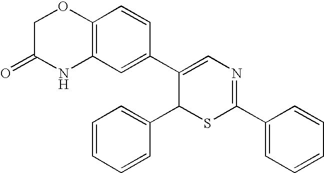

According to a method similar to the procedure for 6-(2-methyl-6-phenyl-6H-1,3-thiazin-5-yl)-2H-1,4-benzoxazin-3(4H)-one, 2-(3-oxo-3,4-dihydro-2H-1,4-benzoxazin-6-yl)-3-phenylacrylaldehyde (0.2 g) was reacted with benzenecarbothioamide (0.11 g) to give the title compound as crystals (0.175 g).

mp. 231-233° C. (ethyl acetate).

¹H-NMR (300 MHz, DMSO-d₆) δ: 4.57 (s, 2H), 5.61 (s, 1H), 6.95 (d, J=8.5 Hz, 1H), 7.09 (d, J=2.3 Hz, 1H), 7.13-7.56 (m, 9H), 7.79-7.91 (m, 3H), 10.76 (s, 1H).

### Example 190

### 6-(2-Ethyl-6-phenyl-6H-1,3-thiazin-5-yl)-2H-1,4-benzoxazin-3(4H)-one

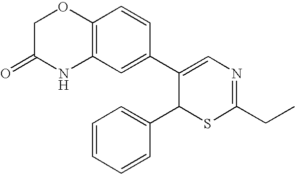

According to a method similar to the procedure for 6-(2-methyl-6-phenyl-6H-1,3-thiazin-5-yl)-2H-1,4-benzoxazin-3(4H)-one, 2-(3-oxo-3,4-dihydro-2H-1,4-benzoxazin-6-yl)-3-phenylacrylaldehyde (0.2 g) was reacted with propanethioamide (70 mg) to give the title compound as crystals (0.11 g).

mp. 130-134° C. (ethyl acetate/hexane).

¹H-NMR (300 MHz, DMSO-d₆) δ: 0.98 (t, J=7.6 Hz, 3H), 2.30-2.49 (m, 2H), 4.54 (s, 2H), 5.35 (s, 1H), 6.90 (d, J=8.3 Hz, 1H), 6.99 (d, J=2.3 Hz, 1H), 7.07 (dd, J=8.3, 2.3 Hz, 1H), 7.17-7.34 (m, 5H), 7.48 (s, 1H), 10.69 (s, 1H).

### Example 191

### 6-[2-(5-Hydroxypentyl)-6-phenyl-6H-1,3-thiazin-5-yl]-2H-1,4-benzoxazin-3(4H)-one

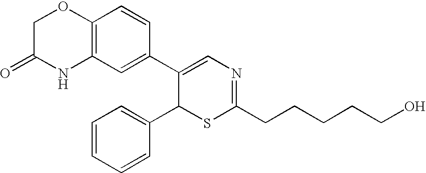

A mixture of 2-(3-oxo-3,4-dihydro-2H-1,4-benzoxazin-6-yl)-3-phenylacrylaldehyde (0.3 g), 6-hydroxyhexanethioamide (0.19 g) and 4N-hydrochloric acid in dioxane (3 mL) was stirred at room temperature for 12 hr. Methanol (3 mL) was added to the mixture, and the mixture was refluxed for 4 hr. The mixture was concentrated in vacuo, and then saturated aqueous sodium bicarbonate solution and water were added to the residue. The mixture was extracted with ethyl acetate, and the organic layer was washed with brine, dried over Na₂SO₄ and concentrated in vacuo. The residue was purified by chromatography on silica gel (hexane→ethyl acetate) and basic silica gel (hexane→ethyl acetate) to give the title compound as an amorphous powder (0.13 g).

¹H-NMR (300 MHz, DMSO-d₆) δ: 1.01-1.51 (m, 6H) 2.37 (t, J=7.25 Hz, 2H) 3.21-3.31 (m, 2H) 4.28 (t, J=5.2 Hz, 1H) 4.54 (s, 2H) 5.35 (s, 1H) 6.90 (d, J=8.5 Hz, 1H) 6.99 (d, J=2.1 Hz, 1H) 7.06 (dd, J=8.5, 2.1 Hz, 1H) 7.15-7.35 (m, 5H) 7.49 (s, 1H) 10.69 (s, 1H).

### Preparation 119

### 6-Bromo-2H-pyrido[3,2-b][1,4]oxazin-3(4H)-one

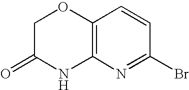

To a solution of 2-nitropyridin-3-ol (30.0 g) in MeOH (500 mL) was added NaOMe (28% MeOH solution, 37.2 g) at r.t. After stirring 30 min at r.t., the mixture was cooled to 0° C. Br₂ (30.8 g) was added to the mixture slowly. After stirring 30 min at 0° C., the reaction mixture was quenched with AcOH. The solvent was removed in vacuo. The residue was dissolved in EtOAc, washed with brine, dried over Na₂SO₄ and concentrated in vacuo. The residue was dissolved in acetone (500 mL). Ethyl bromoacetate (42.9 g) and K₂CO₃ (44.4 g) were added to the acetone solution. After stirring 12 hr under reflux, the reaction mixture was concentrated in vacuo. The residue was treated with EtOAc and H₂O. The organic layer was separated, washed with brine, dried over Na₂SO₄ and concentrated in vacuo. The residue was dissolved with 80% aqueous EtOH (500 mL). Fe (59.8 g) and CaCl₂ (2.38 g) were added to the EtOH solution. After stirring for 3 hr at 80° C., the reaction mixture was filtered through filter paper. The filtrate was concentrated in vacuo. The residue was treated with EtOAc and H₂O. The organic layer was separated, washed with brine, dried over Na₂SO₄ and concentrated in vacuo. The residue was recrystallized from EtOAc and hexane to give the title compound (27.0 g).

¹H-NMR (300 MHz, DMSO-d₆) δ: 4.66 (s, 2H), 7.16 (d, J=8.0 Hz, 1H), 7.31 (d, J=8.0 Hz, 1H), 11.50 (s, 1H).

### Preparation 120

### 2-(3-Oxo-3,4-dihydro-2H-pyrido[3,2-b][1,4]oxazin-6-yl)-3-phenylacrylaldehyde

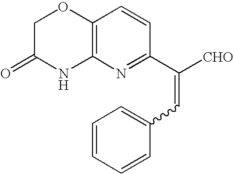

A mixture of 6-bromo-2H-pyrido[3,2-b][1,4]oxazin-3(4H)-one (4.00 g), bis(pinacolato)diboron (4.89 g), [1,1-bis(diphenylphosphino)ferrocene]dichloropalladium(II) dichloromethane adduct (2.15 g) and potassium acetate (6.01 g) in degassed 1,4-dioxane (160 mL) was stirred at 90° C. for 13 hr under an argon atmosphere. The reaction mixture was treated with EtOAc and H₂O. The organic layer was separated, washed with brine, dried over Na₂SO₄ and concentrated in vacuo. The residue was dissolved with degassed solvent of THF (150 mL) and H₂O (30 mL). To the solution were added c-bromocinnamaldehyde (3.69 g), [1,1-bis(diphenylphosphino)ferrocene]dichloropalladium(II) dichloromethane adduct (3.57 g) and Cs₂CO₃ (17.1 g) at r.t. After stirring under reflux for 13 hr under an argon atmosphere, the reaction mixture was treated with EtOAc and H₂O. The organic layer was separated, washed with brine, dried over Na₂SO₄ and concentrated in vacuo. The residue was purified by silica gel column chromatography using hexane/EtOAc as an eluent to give the title compound (765 mg).

¹H-NMR (300 MHz, DMSO-d₆) δ: 4.71 (s, 2H), 6.82 (d, J=8.0 Hz, 1H), 7.17-7.25 (m, 2H), 7.30-7.44 (m, 4H), 7.74 (s, 1H), 9.75 (s, 1H), 11.32 (s, 1H).

### Preparation 121

### 6-(2-Amino-6-phenyl-6H-1,3-thiazin-5-yl)-2H-pyrido[3,2-b][1,4]oxazin-3(4H)-one

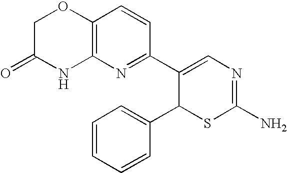

A solution of thiourea (250 mg) and 2-(3-oxo-3,4-dihydro-2H-pyrido[3,2-b][1,4]oxazin-6-yl)-3-phenylacrylaldehyde (765 mg) in a mixture of conc. HCl (3.0 mL), H₂O (6.0 mL) and 1,4-dioxane (30 mL) was stirred for 12 hr under reflux. The reaction mixture was treated with EtOAc and 1N NaOH. The organic layer was separated, washed with brine, dried over Na₂SO₄ and concentrated in vacuo. The residue was purified by silica gel column chromatography using hexane/EtOAc as an eluent to give the title compound (44.7 mg).

¹H-NMR (300 MHz, DMSO-d₆) δ: 4.57 (s, 2H), 5.58 (s, 1H), 7.03-7.28 (m, 9H), 7.74 (s, 1H), 11.08 (s, 1H).

### Example 192

### 6-(7-Phenyl-7H-imidazo[2,1-b][1,3]thiazin-6-yl)-2H-pyrido[3,2-b][1,4]oxazin-3(4H)-one

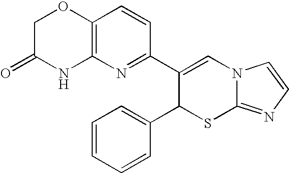

A solution of chloroacetaldehyde (45% aqueous solution, 180 mg) and 6-(2-amino-6-phenyl-6H-1,3-thiazin-5-yl)-2H-pyrido[3,2-b][1,4]oxazin-3(4H)-one (44.0 mg) in a mixture of EtOH (15 mL) and 1,2-dimethoxyethane (15 mL) was stirred for 12 hr under reflux. The reaction mixture was treated with EtOAc and 1N NaOH. The organic layer was separated, washed with brine, dried over Na₂SO₄ and concentrated in vacuo. The residue was purified by reversed phase high-performance liquid chromatography using H₂O/acetonitrile as an eluent and recrystallized from EtOAc and hexane to give the title compound (23 mg).

¹H-NMR (300 MHz, DMSO-d₆) δ: 4.65 (s, 2H), 5.89 (s, 1H), 7.00 (d, J=1.5 Hz, 1H), 7.16-7.30 (m, 6H), 7.37 (d, J=8.5 Hz, 1H), 7.62 (d, J=1.5 Hz, 1H), 8.22 (s, 1H), 11.29 (s, 1H).

### Example 193

### 8-Fluoro-6-[7-(4-fluorophenyl)-2,3-dihydro-7H-imidazo[2,1-b][1,3]thiazin-6-yl]-2H-1,4-benzoxazin-3(4H)-one

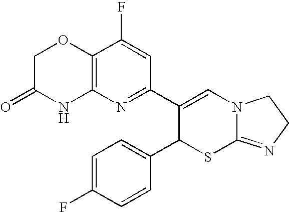

A solution of 2-(8-fluoro-3-oxo-3,4-dihydro-2H-1,4-benzoxazin-6-yl)-3-(4-fluorophenyl)acrylaldehyde (66.0 mg) and imidazolidine-2-thione (26.0 mg) in conc. HCl (1.5 mL), H₂O (3.0 mL) and 1,4-dioxane (15 mL) was stirred for 12 hr under reflux. The reaction mixture was treated with THF, EtOAc and H₂O. The organic layer was separated, washed with brine, dried over Na₂SO₄ and concentrated in vacuo. The residue was purified by silica gel column chromatography using hexane/EtOAc as an eluent and recrystallized from THF, EtOAc and hexane to give the title compound (47.5 mg).

¹H-NMR (300 MHz, DMSO-d₆) δ: 3.65-3.95 (m, 4H), 4.59 (s, 2H), 5.41 (s, 1H), 6.48-6.58 (m, 1H), 6.89 (dd, J=12.5, 2.0 Hz, 1H), 7.09-7.24 (m, 3H), 7.29-7.42 (m, 2H), 10.81 (brs, 1H).

### Example 194

### 8-Fluoro-6-[2-(4-fluorophenyl)-7,8-dihydro-2H,6H-pyrimido[2,1-b][1,3]thiazin-3-yl]-2H-1,4-benzoxazin-3(4H)-one

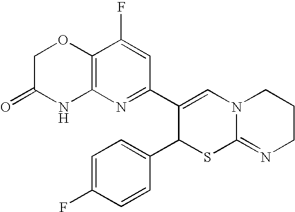

The title compound (10.0 mg) was obtained from 2-(8-fluoro-3-oxo-3,4-dihydro-2H-1,4-benzoxazin-6-yl)-3-(4-fluorophenyl)acrylaldehyde (37.0 mg) according to a method similar to the procedure for Example 193.

¹H-NMR (300 MHz, DMSO-d₆) δ: 1.73-1.96 (m, 2H), 3.25-3.41 (m, 2H), 3.62-3.80 (m, 2H), 4.59 (s, 2H), 5.13 (s, 1H), 6.51-6.55 (m, 1H), 6.83 (s, 1H), 6.91 (dd, J=12.5, 2.0 Hz, 1H), 7.16 (t, J=9.0 Hz, 2H), 7.33 (dd, J=9.0, 5.5 Hz, 2H), 10.81 (brs, 1H).

### Preparation 122

### 1-(3-Fluoro-4-hydroxyphenyl)-2-(4-fluorophenyl)ethanone

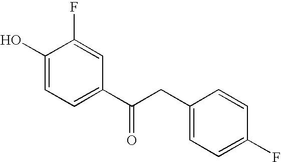

To a solution of (4-fluorophenyl)acetic acid (13.0 g) in THF (100 mL) and DMF (870 μL) was added oxalyl chloride (8.73 mL) at 0° C. After stirring for 2 hr at r.t., the reaction solvent was removed in vacuo. The residue was dissolved in CH₂Cl₂ (20 mL) and added to a suspension of 2-fluoroanisole (10.6 g) and AlCl₃ (33.5 g) in CH₂Cl₂ (100 mL) at 0° C. After stirring for 12 hr at r.t., the reaction mixture was poured into ice-water. The mixture was treated with Et₂O, EtOAc and H₂O. The organic layer was separated, washed with brine, dried over Na₂SO₄ and concentrated in vacuo. The residue was purified by silica gel column chromatography using hexane/EtOAc as an eluent to give the title compound (12.0 g).

¹H-NMR (300 MHz, DMSO-d₆) δ: 4.30 (s, 2H), 6.98-7.18 (m, 3H), 7.20-7.34 (m, 2H), 7.71-7.85 (m, 2H), 10.94 (brs, 1H).

### Preparation 123

### 8-Fluoro-6-[(4-fluorophenyl)acetyl]-2H-1,4-benzoxazin-3(4H)-one

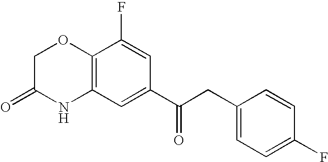

To a suspension of 1-(3-fluoro-4-hydroxyphenyl)-2-(4-fluorophenyl)ethanone (11.9 g) in propionic acid (240 mL) and conc. H₂SO₄ (640 μL) were added NaNO₂ (1.3 mg) and HNO₃ (70%, 2.76 mL) at r.t. After stirring for 2.5 hr at r.t., the reaction mixture was diluted with H₂O. The precipitate was collected by filtration, washed with H₂O and dried in vacuo. The precipitate (8.36 g) was dissolved in a mixture of AcOH (50 mL) and THF (50 mL). Zu dust (20.7 g) was added to the mixture at 50° C. After stirring for 1 hr at 60° C., the reaction mixture was filtered through filter paper. The filtrate was concentrated in vacuo. The residue was dissolved with EtOAc, washed with H₂O, brine, dried over Na₂SO₄ and concentrated in vacuo. The residue was dissolved with a biphasic mixture of 4-methyl-2-pentanone (250 mL) and H₂O (250 mL). To the mixture were added Na₂CO₃ (4.71 g) and chloroacetyl chloride (5.02 g) at r.t. After stirring for 1.5 hr under reflux, the mixture was extracted with EtOAc. The organic extract was washed with brine, dried over Na₂SO₄ and concentrated in vacuo. The residue was purified by silica gel column chromatography using hexane/EtOAc as an eluent to give the title compound (6.17 g).

¹H-NMR (300 MHz, DMSO-d₆) δ: 4.31 (s, 2H), 4.77 (s, 2H), 7.09-7.19 (m, 2H), 7.23-7.32 (m, 2H), 7.34-7.38 (m, 1H), 7.68 (dd, J=11.0, 2.0 Hz, 1H), 11.05 (brs, 1H).

### Preparation 124

### 8-Chloro-6-[(4-fluorophenyl)acetyl]-2H-1,4-benzoxazin-3(4H)-one

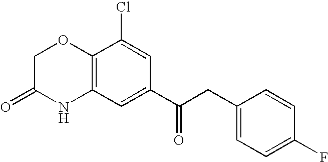

The title compound (5.83 g) was obtained from (4-chlorophenyl)acetic acid (17.6 g) according to a method similar to the procedure for Preparation 122 and 123.

¹H-NMR (300 MHz, DMSO-d₆) δ: 4.34 (s, 2H), 4.82 (s, 2H), 7.10-7.19 (m, 2H), 7.23-7.32 (m, 2H), 7.45 (d, J=2.0 Hz, 1H), 7.84 (d, J=2.0 Hz, 1H), 11.05 (brs, 1H).

### Example 195

### 8-Fluoro-6-[7-(4-fluorophenyl)-7H-[1,2,4]triazolo[3,4-b][1,3,4]thiadiazin-6-yl]-2H-1,4-benzoxazin-3(4H)-one

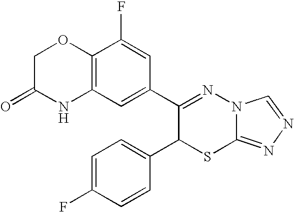

To a suspension of 8-fluoro-6-[(4-fluorophenyl)acetyl]-2H-1,4-benzoxazin-3(4H)-one (776 mg) and pyridinium tribromide (900 mg) in AcOH (16 mL) was added 25% HBr in AcOH (4 mL) at r.t. After stirring for 2.5 hr at r.t., the reaction mixture was treated with EtOAc and H₂O. The organic layer was separated, washed with aq. Na₂S₂O₃ solution, aq. NaHCO₃ solution and brine, dried over Na₂SO₄ and concentrated in vacuo. The residue and 4-amino-4H-1,2,4-triazole-3-thiol (327 mg) were dissolved in a mixture of toluene (30 mL) and EtOH (30 mL). The mixture was stirred for 12 hr under reflux and treated with EtOAc, THF and 1N NaOH at r.t. The organic layer was separated, washed with brine, dried over Na₂SO₄ and concentrated in vacuo. The residue was recrystallized from THF and hexane to give the title compound (600 mg).

¹H-NMR (300 MHz, DMSO-d₆) δ: 4.76 (s, 2H), 6.32 (s, 1H), 7.15-7.23 (m, 4H), 7.33-7.40 (m, 1H), 7.49 (dd, J=11.5, 2.0 Hz, 1H), 9.26 (s, 1H), 11.11 (s, 1H).

### Example 196

### 8-Fluoro-6-[2-(4-fluorophenyl)-7-(trifluoromethyl)-2H-imidazo[2,1-b][1,3,4]thiadiazin-3-yl]-2H-1,4-benzoxazin-3(4H)-one

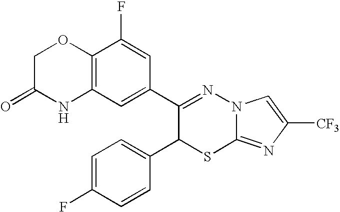

The title compound (397 mg) was obtained from 8-fluoro-6-[(4-fluorophenyl)acetyl]-2H-1,4-benzoxazin-3(4H)-one (320 mg) according to a method similar to the procedure for Preparation 14 and Example 7.

¹H-NMR (300 MHz, DMSO-d₆) δ: 4.76 (s, 2H), 6.28 (s, 1H), 7.14-7.27 (m, 4H), 7.33-7.37 (m, 1H), 7.47 (dd, J=11.5, 2.0 Hz, 1H), 8.53-8.57 (m, 1H), 11.13 (s, 1H).

### Example 197

### 8-Fluoro-6-[2-(4-fluorophenyl)-7-methyl-2H-imidazo[2,1-b][1,3,4]thiadiazin-3-yl]-2H-1,4-benzoxazin-3(4H)-one

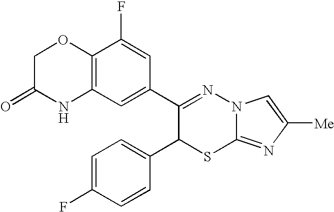

The title compound (347 mg) was obtained from 8-fluoro-6-[(4-fluorophenyl)acetyl]-2H-1,4-benzoxazin-3(4H)-one (345 mg) according to a method similar to the procedure for Preparation 14 and Example 6.

mp. 170.1-172.2° C.

¹H-NMR (300 MHz, DMSO-d₆) δ: 2.08 (d, J=1.0 Hz, 3H), 4.74 (s, 2H), 6.10 (s, 1H), 7.14-7.21 (m, 4H), 7.32-7.36 (m, 1H), 7.42 (dd, J=11.5, 2.0 Hz, 1H), 7.49 (d, J=1.0 Hz, 1H), 11.06 (brs, 1H).

### Preparation 125

### 6-[Bromo(4-fluorophenyl)acetyl]-8-chloro-2H-1,4-benzoxazin-3(4H)-one

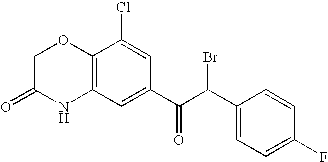

The title compound (2.70 g) was obtained from 8-chloro-6-[(4-fluorophenyl)acetyl]-2H-1,4-benzoxazin-3(4H)-one (4.66 g) according to a method similar to the procedure for Preparation 14.

¹H-NMR (300 MHz, DMSO-d₆) δ: 4.82 (s, 2H), 7.11 (s, 1H), 7.19-7.29 (m, 2H), 7.46 (d, J=2.0 Hz, 1H), 7.55-7.65 (m, 2H), 7.95 (d, J=2.0 Hz, 1H), 11.09 (s, 1H).

### Example 198

### 8-Chloro-6-[7-(4-fluorophenyl)-7H-[1,2,4]triazolo[3,4-b][1,3,4]thiadiazin-6-yl]-2H-1,4-benzoxazin-3(4H)-one

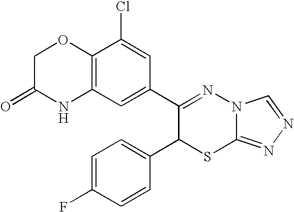

The title compound (797 mg) was obtained from 6-[bromo(4-fluorophenyl)acetyl]-8-chloro-2H-1,4-benzoxazin-3(4H)-one (1.82 g) according to a method similar to the procedure for Example 3.

¹H-NMR (300 MHz, DMSO-d₆) δ: 4.80 (s, 2H), 6.37 (s, 1H), 7.16-7.23 (m, 4H), 7.49 (d, J=2.0 Hz, 1H), 7.64 (d, J=2.0 Hz, 1H), 9.27 (s, 1H), 11.12 (brs, 1H).

### Example 199

### 8-Chloro-6-[2-(4-fluorophenyl)-7-(trifluoromethyl)-2H-imidazo[2,1-b][1,3,4]thiadiazin-3-yl]-2H-1,4-benzoxazin-3(4H)-one

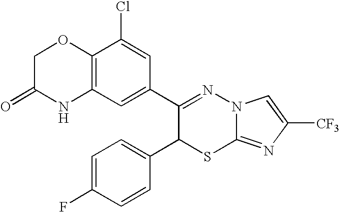

The title compound (399 mg) was obtained from 6-[bromo(4-fluorophenyl)acetyl]-8-chloro-2H-1,4-benzoxazin-3(4H)-one (183 mg) according to a method similar to the procedure for Example 7.

¹H-NMR (300 MHz, DMSO-d₆) δ: 4.80 (s, 2H), 6.33 (s, 1H), 7.17-7.24 (m, 4H), 7.47 (d, J=2.0 Hz, 1H), 7.62 (d, J=2.0 Hz, 1H), 8.55-8.58 (m, 1H), 11.14 (brs, 1H).

### Example 200

### 6-(2-Methyl-5-phenylpyrimidin-4-yl)-2H-1,4-benzoxazin-3(4H)-one

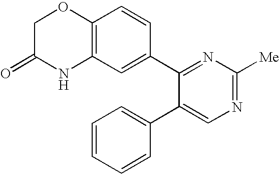

To a suspension of 6-(phenylacetyl)-2H-1,4-benzoxazin-3(4H)-one (550 mg) in dry THF (10 mL) was added N,N-dimethylformamide dimethyl acetal (733 mg) at r.t. After stirring for 3 hr at 60° C., the reaction mixture was diluted with hexane. The precipitate was collected by filtration, washed with hexane and dried in vacuo. The precipitate was dissolved in a mixture of EtOH (20 mL) and THF (20 mL). Acetamidine hydrochloride (580 mg) and potassium tert-butoxide (1.15 g) were added to the mixture. After stirring for 12 hr under reflux, the reaction solvent was removed in vacuo. The residue was dissolved with EtOAc, washed with brine, dried over Na₂SO₄ and concentrated in vacuo. The residue was purified by silica gel column chromatography using hexane/EtOAc as an eluent and recrystallized from EtOAc and hexane to give the title compound (281 mg).

mp. 229.0-229.1° C.

¹H-NMR (300 MHz, DMSO-d₆) δ: 2.70 (s, 3H), 4.60 (s, 2H), 6.71 (dd, J=8.5, 2.0 Hz, 1H), 6.78 (d, J=8.5 Hz, 1H), 7.22 (d, J=8.5 Hz, 1H), 7.23-7.30 (m, 2H), 7.34-7.45 (m, 3H), 8.64 (s, 1H), 10.76 (s, 1H).

### Example 201

### 6-(2-Methoxy-2-phenyl-2H-imidazo[2,1-b][1,3,4]thiadiazin-3-yl)-2H-1,4-benzoxazin-3(4H)-one

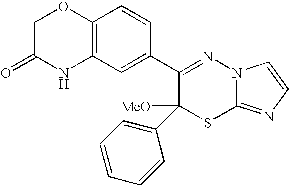

A mixture of 6-(2-phenyl-2H-imidazo[2,1-b][1,3,4]thiadiazin-3-yl)-2H-1,4-benzoxazin-3(4H)-one (100 mg) and 65% m-chloroperbenzoic acid (68 mg) in methanol (6 mL) was, stirred for 3 days. The solvent was removed and the residue was treated with THF, saturated aqueous NaHCO₃ and saturated aqueous Na₂SO₃. The organic layer was separated and the aqueous layer was extracted with ethyl acetate. The organic layers were combined, dried over MgSO₄ and concentrated in vacuo. The residue was chromatographed on basic silica gel with hexane/ethyl acetate as an eluent to give the title compound. Recrystallization from ethyl acetate/hexane gave colorless crystals (46 mg).

¹H-NMR (300 MHz, DMSO-d₆) δ: 3.30 (s, 3H), 4.57 (s, 2H), 6.79-6.82 (m, 1H), 6.99-7.03 (m, 1H), 7.10-7.11 (m, 1H), 7.17-7.18 (m, 1H), 7.26-7.36 (m, 3H), 7.45-7.49 (m, 2H), 7.84-7.85 (m, 1H), 10.77 (brs, 1H). MS (ESI) m/z 393 (M+1)

### Preparation 126

### Ethyl 6-(3-oxo-3,4-dihydro-2H-1,4-benzoxazin-6-yl)-7-phenyl-7H-imidazo[2,1-b][1,3]thiazine-2-carboxylate

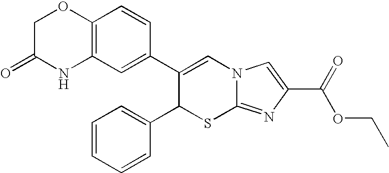

A suspension of 6-(2-amino-6-phenyl-6H-1,3-thiazin-5-yl)-2H-1,4-benzoxazin-3(4H)-one (1.0 g) and ethyl bromopyruvate (1.16 g) in ethanol (10 ml) was stirred at reflux for 27 hr. Then, ethyl bromopyruvate (0.58 g) was added and the mixture was stirred for additional 3 hr. The mixture was treated with water and ethyl acetate. The organic layer was separated and the aqueous layer was extracted with ethyl acetate. The organic layers were combined, dried over MgSO₄ and concentrated in vacuo. The residue was chromatographed on silica gel with hexane/ethyl acetate as an eluent to give the title compound. Recrystallization from THF/ethanol gave colorless crystals (360 mg).

¹H-NMR (300 MHz, DMSO-d₆) δ: 1.25 (t, J=7.0 Hz, 3H), 4.21 (q, J=7.0 Hz, 2H), 4.57 (s, 2H), 5.64 (s, 1H), 6.83-7.14 (m, 3H), 7.16-7.45 (m, 5H), 7.84 (s, 1H), 8.27 (s, 1H), 10.82 (brs, 1H). MS (ESI) m/z 434 (M+1)

### Preparation 127

### 6-(3-Oxo-3,4-dihydro-2H-1,4-benzoxazin-6-yl)-7-phenyl-7H-imidazo[2,1-b][1,3]thiazine-2-carboxamide

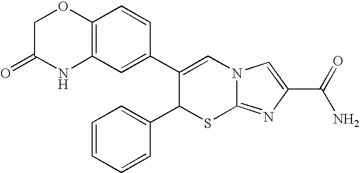

To a suspension of ethyl 6-(3-oxo-3,4-dihydro-2H-1,4-benzoxazin-6-yl)-7-phenyl-7H-imidazo[2,1-b][1,3]thiazine-2-carboxylate (1.66 g) in ethanol (40 mL) was added 3N aqueous sodium hydroxide solution (26 mL) at r.t. The mixture was stirred for 3 hr, adjusted to pH 7 with conc. HCl and extracted with ethyl acetate. The organic layer was dried over MgSO₄ and the solvent was removed in vacuo to give a 6-(3-oxo-3,4-dihydro-2H-1,4-benzoxazin-6-yl)-7-phenyl-7H-imidazo[2,1-b][1,3]thiazine-2-carboxylic acid. The carboxylic acid was dissolved in DMF (200 mL). WSC (1.1 g) and 1H-1,2,3-benzotriazol-1-ol ammoniate (0.699 g) were added. Then, the mixture was stirred at r.t. for 12 hr. The solvent was removed in vacuo. The residue was treated with ethyl acetate and saturated aqueous NaHCO₃ The organic layer was dried over MgSO₄ and concentrated in vacuo. The residual solid was suspended in ethyl acetate/diisopropyl ether and then collected by filtration to give the title compound as amorphous solid (1.32 g).

¹H-NMR (300 MHz, DMSO-d₆) δ: 4.57 (s, 2H), 5.62 (s, 1H), 6.87-7.07 (m, 3H), 7.19-7.46 (m, 7H), 7.86 (s, 1H), 8.02 (s, 1H), 10.80 (brs, 1H). MS (ESI) m/z 405 (M+1)

### Example 202

### 6-(3-Oxo-3,4-dihydro-2H-1,4-benzoxazin-6-yl)-7-phenyl-7H-imidazo[2,1-b][1,3]thiazine-2-carbonitrile

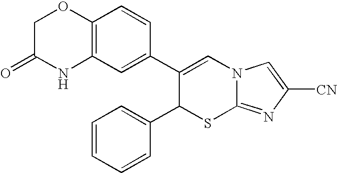

To a stirred suspension of 6-(3-oxo-3,4-dihydro-2H-1,4-benzoxazin-6-yl)-7-phenyl-7H-imidazo[2,1-b][1,3]thiazine-2-carboxamide (500 mg) in pyridine/dioxane (0.3 ml/5 mL) was added trifluoroacetic anhydride (519 mg) at 0° C. The mixture was stirred for 20 min and treated with water and ethyl acetate. The organic layer was separated and the aqueous layer was extracted with ethyl acetate. The organic layers were combined, dried over MgSO₄ and concentrated in vacuo. The residue was chromatographed on basic silica gel with hexane/ethyl acetate as an eluent to give the title compound. Recrystallization from ethanol gave colorless crystals (258 mg).

¹H-NMR (300 MHz, DMSO-d₆) δ: 4.57 (s, 2H), 5.73 (s, 1H), 6.94-7.00 (m, 2H), 7.06-7.07 (m, 1H), 7.23-7.34 (m, 5H), 7.87 (s, 1H), 8.48 (s, 1H), 10.82 (brs, 1H). MS (ESI) m/z 387 (M+1)

### Example 203

### 6-(2-Ethyl-7-phenyl-7H-imidazo[2,1-b][1,3]thiazin-6-yl)-2H-1,4-benzoxazin-3(4H)-one

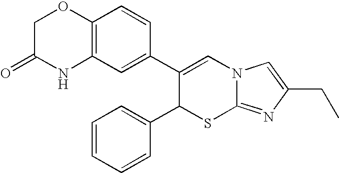

A mixture of 6-(2-amino-6-phenyl-6H-1,3-thiazin-5-yl)-2H-1,4-benzoxazin-3(4H)-one (150 mg) and 1-bromo-2-butanone (45.4 μL) in 1,2-dimethoxyethane/ethanol (5 ml/1 mL) was stirred at reflux for 12 hr and treated with ethyl acetate and saturated aqueous NaHCO₃. The organic layer was separated and the aqueous layer was extracted with ethyl acetate. The organic layers were combined, dried over MgSO₄ and concentrated in vacuo. The residue was purified roughly by preparative HPLC and then chromatographed on silica gel with ethyl acetate/hexane as an eluent to give the title compound. Recrystallization from ethyl acetate/hexane gave colorless crystals (3 mg).

¹H-NMR (300 MHz, DMSO-d₆) δ: 1.11 (t, J=7.6 Hz, 3H), 2.42 (q, J=7.6 Hz, 2H), 4.55 (s, 2H), 5.49 (s, 1H), 6.90-7.03 (m, 3H), 7.21-7.33 (m, 6H), 7.72 (s, 1H), 10.78 (brs, 1H). MS (ESI) m/z 390 (M+1)

### Example 204

### [6-(3-Oxo-3,4-dihydro-2H-1,4-benzoxazin-6-yl)-7-phenyl-7H-imidazo[2,1-b][1,3]thiazin-2-yl]methyl acetate

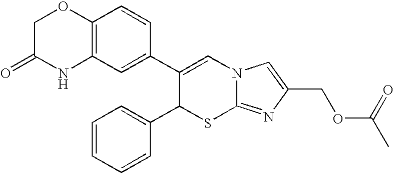

A mixture of 6-(2-amino-6-phenyl-6H-1,3-thiazin-5-yl)-2H-1,4-benzoxazin-3(4H)-one (200 mg) and 1-acetoxy-3-chloroacetone (134 mg) in 1,2-dimethoxyethane (10 mL) was stirred at 100° C. for 12 hr and treated with ethyl acetate and saturated aqueous NaHCO₃. The organic layer was separated and the aqueous layer was extracted with ethyl acetate. The organic layers were combined, dried over MgSO₄ and concentrated in vacuo. The residue was chromatographed on silica gel with ethyl acetate/hexane as an eluent to give the title compound (100 mg) as a foamy solid.

¹H-NMR (300 MHz, DMSO-d₆) δ: 2.04 (s, 3H), 4.57 (s, 2H), 4.92-5.05 (m, 3H), 6.84-6.89 (m, 3H), 7.18-7.28 (m, 7H), 9.76 (brs, 1H).

### Example 205

### 6-[2-(Hydroxymethyl)-7-phenyl-7H-imidazo[2,1-b][1,3]thiazin-6-yl]-2H-1,4-benzoxazin-3(4H)-one

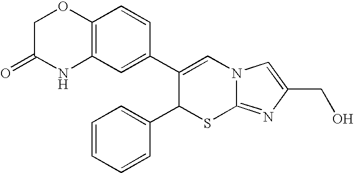

A mixture of [6-(3-oxo-3,4-dihydro-2H-1,4-benzoxazin-6-yl)-7-phenyl-7H-imidazo[2,1-b][1,3]thiazin-2-yl]methyl acetate (90 mg) and K₂CO₃ (57.4 mg) in methanol (2 mL) was stirred at r.t. for 12 hr and treated with ethyl acetate and water. The organic layer was separated and the aqueous layer was extracted with ethyl acetate. The organic layers were combined, dried over MgSO₄ and concentrated in vacuo. The residue was purified by preparative HPLC to give the title compound (40 mg) as foamy solid.

¹H-NMR (300 MHz, DMSO-d₆) δ: 4.29 (d, J=5.3 Hz, 2H), 4.56 (s, 2H), 4.99 (t, J=5.3 Hz, 1H), 5.51 (s, 1H), 6.91-7.04 (m, 3H), 7.19-7.34 (m, 5H), 7.41 (s, 1H), 7.85 (s, 1H), 10.77 (brs, 1H).

### Example 206

### 6-(2-Amino-6-phenyl-6H-1,3,4-thiadiazin-5-yl)-2H-1,4-benzoxazin-3(4H)-one

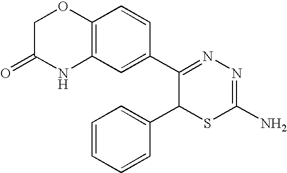

A mixture of 6-[bromo(phenyl)acetyl]-2H-1,4-benzoxazin-3(4H)-one (2.0 g) and thiosemicarbazide (0.48 g) was stirred at reflux for 3 hr, treated with THF and saturated aqueous NaHCO₃. The organic layer was separated and the aqueous layer was extracted with THF. The organic layers were combined, dried over MgSO₄ and concentrated in vacuo. The residue was chromatographed on silica gel with ethyl acetate/hexane as an eluent to give the title compound as a white solid (5 mg).

¹H-NMR (300 MHz, DMSO-d₆) δ: 4.58 (s, 2H), 5.62 (s, 1H), 6.66 (brs, 2H), 6.92-6.95 (m, 1H), 7.12-7.29 (m, 6H), 7.56-7.57 (m, 1H), 10.79 (brs, 1H).

### Example 207

### 6-[2-(Ethylamino)-6-phenyl-6H-1,3-thiazin-5-yl]-2H-1,4-benzoxazin-3(4H)-one

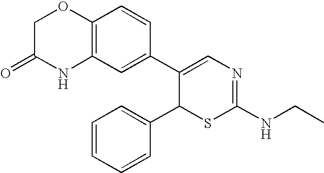

A mixture of (2E)-2-(3-oxo-3,4-dihydro-2H-1,4-benzoxazin-6-yl)-3-phenylacrylaldehyde (150 mg), 1-ethyl-2-thiourea (48.4 mg), 1,4-dioxane (10 mL), water (2 mL) and conc. HCl (1 mL) was stirred at reflux for 3 hr, and then treated with ethyl acetate and saturated aqueous NaHCO₃. The organic layer was separated and the aqueous layer was extracted with ethyl acetate. The organic layers were combined, dried over MgSO₄ and concentrated in vacuo. The residue was crystallized from ethanol to give the title compound (27 mg).

¹H-NMR (300 MHz, DMSO-d₆) δ: 1.03 (t, J=7.2 Hz, 3H), 3.22-3.31 (m, 2H), 4.50 (s, 2H), 5.17 (s, 1H), 6.81-6.90 (m, 3H), 7.16-7.31 (m, 7H), 10.64 (brs, 1H). MS (ESI) m/z: 366 (M+1).

### Example 208

### 6-[2-(Methylamino)-6-phenyl-6H-1,3-thiazin-5-yl]-2H-1,4-benzoxazin-3(4H)-one

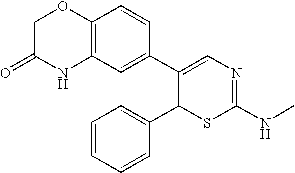

A mixture of (2E)-2-(3-oxo-3,4-dihydro-2H-1,4-benzoxazin-6-yl)-3-phenylacrylaldehyde (200 mg), 1-methyl-2-thiourea (79.2 mg), 1,4-dioxane (10 mL), water (2 mL) and conc. HCl (1 mL) was stirred at reflux for 3 hr, and then treated with ethyl acetate and saturated aqueous NaHCO₃. The organic layer was separated and the aqueous layer was extracted with ethyl acetate. The organic layers were combined, dried over MgSO₄ and concentrated in vacuo. The residue was crystallized from THF/methanol to give the title compound (107 mg).

¹H-NMR (300 MHz, DMSO-d₆) δ: 2.76 (s, 3H), 4.50 (s, 2H), 5.18 (s, 1H), 6.81-6.90 (m, 3H), 7.12-7.31 (m, 7H), 10.65 (brs, 1H). MS (ESI) m/z: 352 (M+1).

### Example 209

### 6-(3-Acetyl-2-imino-6-phenyl-3,6-dihydro-2H-1,3-thiazin-5-yl)-2H-1,4-benzoxazin-3(4H)-one

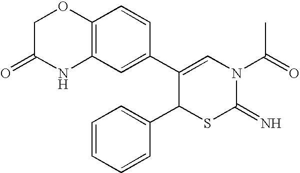

To a stirred mixture of 6-(2-amino-6-phenyl-6H-1,3-thiazin-5-yl)-2H-1,4-benzoxazin-3(4H)-one (100 mg) and triethylamine (42.3 μL) in THF (5 mL) was added acetyl chloride (21.2 μL) at 0° C. The mixture was stirred for 14 hr, and then treated with THF and saturated aqueous NaHCO₃. The organic layer was separated and the aqueous layer was extracted with ethyl acetate. The organic layers were combined, dried over MgSO₄ and filtered. The precipitated crystals were collected to give the title compound (57 mg).

¹H-NMR (300 MHz, DMSO-d₆) δ: 1.95 (s, 3H), 4.53 (s, 2H), 5.19 (s, 1H), 6.86-7.02 (m, 3H), 7.21-7.34 (m, 6H), 10.66 (brs, 1H), 11.04 (brs, 1H). MS (ESI) m/z: 380 (M+1).

### Example 210

### 6-[2-Imino-3-(methylsulfonyl)-6-phenyl-3,6-dihydro-2H-1,3-thiazin-5-yl]-2H-1,4-benzoxazin-3(4H)-one

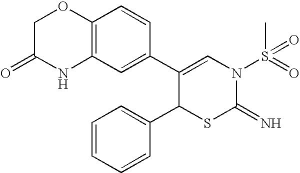

To a stirred mixture of 6-(2-amino-6-phenyl-6H-1,3-thiazin-5-yl)-2H-1,4-benzoxazin-3(4H)-one (50 mg) and triethylamine (41.3 μL) in THF (3 mL) was added a solution of methanesulfonyl chloride (9.6 μL) in THF (1 mL) at 0° C. The mixture was stirred for 14 hr, and then treated with THF and saturated aqueous NaHCO₃. The organic layer was separated and the aqueous layer was extracted with ethyl acetate. The organic layers were combined, dried over MgSO₄ and concentrated. The residue was purified by preparative HPLC to give the title compound. Recrystallization from ethyl acetate gave colorless crystals (4 mg).

¹H-NMR (300 MHz, DMSO-d₆) δ: 2.78 (s, 3H), 4.53 (s, 2H), 5.43 (brs, 1H), 6.86-6.91 (m, 4H), 7.28-7.36 (m, 5H), 10.68 (brs, 1H), 11.08 (brs, 1H). MS (ESI) m/z: 416 (M+1).

### Example 211

### 6-[7-(2,4-Difluorophenyl)-7H-imidazo[2,1-b][1,3]thiazin-6-yl]-2H-1,4-benzoxazin-3(4H)-one

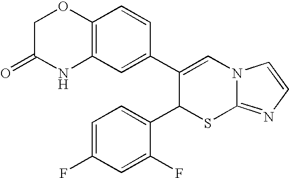

A mixture of 6-[2-amino-6-(2,4-difluorophenyl)-6H-1,3-thiazin-5-yl]-2H-1,4-benzoxazin-3(4H)-one (200 mg) and 45% chloroacetaldehyde (0.748 g) in ethanol/1,2-dimethoxyethane (7 ml/7 mL) was stirred at reflux for 12 hr. The precipitated crystals were collected by filtration, and then were treated with ethyl acetate and saturated aqueous NaHCO₃. The organic layer was separated and the aqueous layer was extracted with THF. The organic layers were combined, dried over MgSO₄ and concentrated in vacuo. The residue was chromatographed on silica gel with ethyl acetate/hexane as an eluent to give the title compound. Recrystallization from methanol gave colorless crystals (30 mg).

¹H-NMR (300 MHz, DMSO-d₆) δ: 4.56 (s, 2H), 5.60 (s, 1H), 6.84-6.85 (m, 1H), 6.93-7.03 (m, 4H), 7.05-7.12 (m, 1H), 7.32-7.44 (m, 1H), 7.59-7.60 (m, 1H), 7.89 (s, 1H), 10.73 (brs, 1H). MS (ESI) m/z: 398 (M+1).

### Example 212

### 6-[2-Amino-6-(2,4-difluorophenyl)-6H-1,3-thiazin-5-yl]-2H-1,4-benzoxazin-3(4H)-one

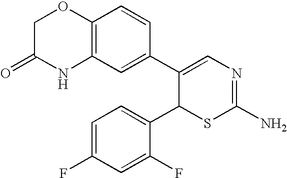

A mixture of (2E)-3-(2,4-difluorophenyl)-2-(3-oxo-3,4-dihydro-2H-1,4-benzoxazin-6-yl)acrylaldehyde (0.80 g), thiourea (0.23 g), 1,4-dioxane (30 mL), water (6 mL) and conc. HCl (3 mL) was stirred at 100° C. for 3 hr, and then treated with THF and saturated aqueous NaHCO₃. The precipitates were collected by filtration and washed with water to give the title compound (647 mg).

¹H-NMR (300 MHz, DMSO-d₆) δ: 4.51 (s, 2H), 5.26 (s, 1H), 6.80-7.10 (m, 7H), 7.26-7.34 (m, 2H), 10.59 (brs, 1H).

MS (ESI) m/z: 374 (M+1).

### Preparation 128

### (2E)-3-(2,4-Difluorophenyl)-2-(3-oxo-3,4-dihydro-2H-1,4-benzoxazin-6-yl)acrylaldehyde

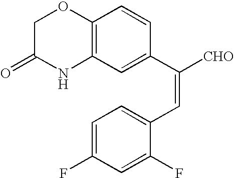

A mixture of (2Z)-3-(2,4-difluorophenyl)-2-iodoacrylaldehyde (2.5 g), 6-(4,4,5,5-tetramethyl-1,3,2-dioxaborolan-2-yl)-2H-1,4-benzoxazin-3(4H)-one (2.34 g), [1,1-bis(diphenylphosphino)ferrocene]dichloropalladium(II) dichloromethane adduct (1.39 g), 2M Cs₂CO₃ (15 mL) and THF (80 mL) was stirred at reflux for 12 hr, and then treated with ethyl acetate and water. The organic layer was separated, dried over MgSO₄ and concentrated in vacuo. The residue was chromatographed on silica gel with hexane/ethyl acetate as an eluent to give the title compound (1.6 g).

¹H-NMR (300 MHz, DMSO-d₆) δ: 4.61 (s, 2H), 6.85-6.69 (m, 2H), 6.94-7.10 (m, 3H), 7.34-7.41 (m, 1H), 7.67 (s, 1H), 9.78 (s, 1H), 10.70 (brs, 1H). MS (ESI) m/z: 315 (M+1).

### Preparation 129

### (2Z)-3-(2,4-Difluorophenyl)-2-iodoacrylaldehyde

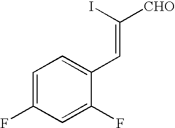

Under nitrogen atmosphere, to a solution of (2Z)-3-(2,4-difluorophenyl)acrylaldehyde (3.6 g) in pyridine/dichloromethane (15 ml/30 mL) was added iodine monochloride (7.0 g) at 0° C. After stirring for 48 hr at r.t, the reaction mixture was quenched with aqueous Na₂S₂O₃ solution and treated with ethyl acetate. The organic layer was separated, washed with 1N HCl solution and brine, dried over MgSO₄ and concentrated in vacuo. The residue was chromatographed on silica gel with hexane/ethyl acetate as an eluent to give the title compound (4.81 g).

¹H-NMR (300 MHz, CDCl₃) δ: 6.89-6.97 (m, 1H), 7.02-7.08 (m, 1H), 8.22 (s, 1H), 8.43-8.51 (m, 1H), 8.80 (s, 1H).

### Preparation 130

### (2Z)-3-(2,4-Difluorophenyl)acrylaldehyde

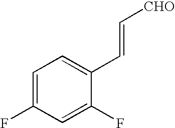

Under nitrogen atmosphere, a mixture of 2,4-difluorobenzaldehyde (500 mg), formylmethylenetriphenylphosphorane (1.39 g) in toluene (20 mL) was stirred at 70° C. for 20 hr. The solvent was removed in vacuo. The residue was chromatographed on silica gel with hexane/ethyl acetate as an eluent to give the title compound (320 mg).

¹H-NMR (300 MHz, DMSO-d₆) δ: 6.74 (dd, J=16.3, 7.57 Hz, 1H), 6.87-7.00 (m, 2H), 7.55-7.63 (m, 2H), 9.71 (d, J=7.57 Hz, 1H).

### Example 213

### 6-(8-Oxido-7-phenyl-7H-imidazo[2,1-b][1,3]thiazin-6-yl)-2H-1,4-benzoxazin-3(4H)-one

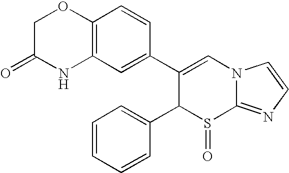

To a solution of 6-(7-phenyl-7H-imidazo[2,1-b][1,3]thiazin-6-yl)-2H-1,4-benzoxazin-3(4H)-one (60 mg) in DMF (5 mL) was added dropwise a solution of 65% m-chloroperbenzoic acid (31.5 mg) in DMF (1 ml) at 0° C. The mixture was stirred for 3 hr, and then treated with ethyl acetate and saturated aqueous NaHCO₃. The organic layer was separated, dried over MgSO₄ and concentrated in vacuo. The residue was crystallized from CH₃CN to give the title compound (26 mg).

¹H-NMR (300 MHz, DMSO-d₆) δ: 4.56 (s, 2H), 5.96 (s, 1H), 6.96-7.06 (m, 3H), 7.22-7.23 (m, 1H), 7.28-7.33 (m, 5H), 7.82 (s, 1H), 8.15 (s, 1H), 10.82 (brs, 1H). MS (ESI) m/z: 378 (M+1).

### Example 214

### 6-(8,8-Dioxido-7-phenyl-7H-imidazo[2,1-b][1,3]thiazin-6-yl)-2H-1,4-benzoxazin-3(4H)-one

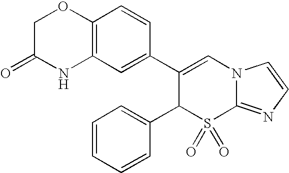

A mixture of 6-(7-phenyl-7H-imidazo[2,1-b][1,3]thiazin-6-yl)-2H-1,4-benzoxazin-3(4H)-one (59.3 mg), 30% hydrogen peroxide (0.15 mL), sulfuric acid (0.1 mL) and acetic acid (1 mL) was stirred for 72 hr, and then treated with ethyl acetate and saturated aqueous NaHCO₃. The organic layer was separated, dried over MgSO₄ and concentrated in vacuo. The residue was purified by preparative HPLC to give the title compound. Recrystallization from ethyl acetate gave colorless crystals (10 mg).

¹H-NMR (300 MHz, DMSO-d₆) δ: 4.57 (s, 2H), 6.12 (s, 1H), 6.94-6.98 (m, 2H), 7.04-7.07 (m, 1H), 7.22-7.26 (m, 2H), 7.32-7.37 (m, 4H), 7.84-7.85 (m, 1H), 8.02 (s, 1H), 10.83 (brs, 1H). MS (ESI) m/z: 394 (M+1).

### Example 215

### 6-[7-(2-Fluorophenyl)-7H-[1,2,4]triazolo[3,4-b][1,3,4]thiadiazin-6-yl]-2H-1,4-benzoxazin-3(4H)-one

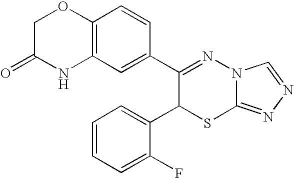

A mixture of 6-[bromo(2-fluorophenyl)acetyl]-2H-1,4-benzoxazin-3(4H)-one (1.0 g) and 4-amino-3-mercapto-4H-1,2,4-triazole (0.34 g) in ethanol/1,2-dimethoxyethane (20 ml/20 mL) was stirred at reflux for 12 hr. The solvent was removed in vacuo and the residue was treated with ethyl acetate and saturated aqueous NaHCO₃. The organic layer was separated and the aqueous layer was extracted with ethyl acetate. The organic layers were combined, dried over MgSO₄ and concentrated in vacuo. The residue was chromatographed on silica gel with ethyl acetate as an eluent to give the title compound. Recrystallization from methanol gave colorless crystals (387 mg).

¹H-NMR (300 MHz, DMSO-d₆) δ: 4.66 (s, 2H), 6.44 (s, 1H), 6.76-6.81 (m, 1H), 7.04-7.10 (m, 2H), 7.30-7.43 (m, 2H), 7.47-7.50 (m, 1H), 7.53-7.54 (m, 1H), 9.28 (s, 1H), 10.92 (brs, 1H). MS (ESI) m/z: 382 (M+1).

### Example 216

### 6-[7-(2,4-Difluorophenyl)-7H-[1,2,4]triazolo[3,4-b][1,3,4]thiadiazin-6-yl]-2H-1,4-benzoxazin-3(4H)-one hydrobromide

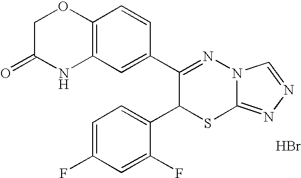

A mixture of 6-[bromo(2,4-difluorophenyl)acetyl]-2H-1,4-benzoxazin-3(4H)-one (1.0 g) and 4-amino-3-mercapto-4H-1,2,4-triazole (0.32 g) in ethanol/dimethoxyethane (20 ml/20 mL) was stirred at reflux for 12 hr. The precipitated crystals were collected by filtration. The crystals were suspended in ethyl acetate and collected by filtration to give the title compound (352 mg).

¹H-NMR (300 MHz, DMSO-d₆) δ: 4.67 (s, 2H), 6.43 (s, 1H), 6.81-6.89 (m, 1H), 6.94-7.00 (m, 1H), 7.06-7.09 (m, 1H), 7.40-7.53 (m, 3H), 9.28 (s, 1H), 10.92 (brs, 1H), 1H unconfirmed.

### Preparation 131

### 6-[Bromo(2,4-difluorophenyl)acetyl]-2H-1,4-benzoxazin-3(4H)-one

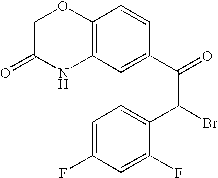

To a mixture of 6-[(2,4-difluorophenyl)acetyl]-2H-1,4-benzoxazin-3(4H)-one (13.5 g), 25% hydrogen bromide in acetic acid (30 mL) and acetic acid (100 mL) was added pyridinium hydrobromide perbromide (14.9 g) at r.t. The mixture was stirred for 2 hr, and then treated with saturated aqueous Na₂S₂O₃ solution (20 mL). Water (200 mL) was added dropwise with stirring to generate white precipitates. The precipitates were collected by filtration, washed with water, suspended in methanol, and then collected by filtration to give the title compound (12.8 g).

¹H-NMR (300 MHz, DMSO-d₆) δ: 4.69 (s, 2H), 7.03-7.06 (m, 1H), 7.12-7.18 (m, 2H), 7.28-7.38 (m, 1H), 7.48-7.49 (m, 1H), 7.57-7.67 (m, 2H), 10.92 (brs, 1H).

### Preparation 132

### 6-[(2,4-Difluorophenyl)acetyl]-2H-1,4-benzoxazin-3(4H)-one

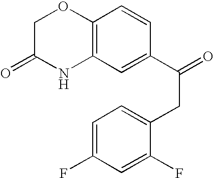

To a mixture of (2,4-difluorophenyl)acetic acid (12 g), DMF (0.5 mL) and THF (100 mL) was added oxalyl chloride (26.5 g) at 0° C. dropwise. The mixture was stirred at r.t. for 1 hr, and then the solvent was evaporated to give (2,4-difluorophenyl)acetyl chloride. To a suspension of 2H-1,4-benzoxazin-3(4H)-one (10.2 g) in nitrobenzene (75 mL) was added aluminum trichloride (21.5 g) at 0° C. To the reaction mixture was added a solution of (2,4-difluorophenyl)acetyl chloride prepared above in nitrobenzene (25 mL) at 0° C. The mixture was stirred for 72 hr at r.t., and then poured into crashed ice. Diisopropyl ether (500 mL) and 1N HCl (50 mL) were added, and then the mixture was stirred for 1 hr. The precipitates were collected by filtration and washed with water to give the title compound (14.1 g).

¹H-NMR (300 MHz, DMSO-d₆) δ: 4.38 (s, 2H), 4.70 (s, 2H), 7.03-7.10 (m, 2H), 7.18-7.26 (m, 1H), 7.33-7.41 (m, 1H), 7.52-7.53 (m, 1H), 7.72-7.76 (m, 1H), 10.90 (brs, 1H).

### Example 217

### 6-(1-(2-Hydroxyethyl)-4-phenyl-4,5-dihydro-1H-pyrazol-3-yl)-2H-benzo[b][1,4]oxazin-3(4H)-one

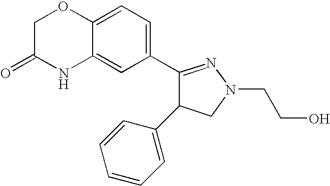

According to the method of Example 46, 6-(2-phenylacryloyl)-2H-benzo[b][1,4]oxazin-3(4H)-one (200 mg, 0.72 mmol) and 2-hydrazinylethanol (81 μL, 1.07 mmol) were reacted to give the title compound, after flash chromatography on silica gel (0-10% MeOH in DCM), as a pale yellow powder (15 mg, 6%).

¹H-NMR (400 MHz, CDCl₃) δ: 8.31 (brs, 1H), 7.27 (m, 2H), 7.23 (m, 3H), 7.18 (d, J=1.6 Hz, 1H), 7.00 (dd, J=8.4, 1.6 Hz, 1H), 6.80 (d, J=8.4 Hz, 1H), 4.56 (s, 2H), 4.45 (dd, J=10.1, 5.1 Hz, 1H), 4.01 (m, 2H), 3.64 (brs, 1H), 3.55 (dd, J=10.1, 9.4 Hz, 1H), 3.42 (dd, J=9.4, 5.1 Hz, 1H); 3.23 (ddd, J=12.6, 7.2, 3.1 Hz, 1H), 3.14 (ddd, J=12.6, 5.6, 3.1 Hz, 1H); LCMS (ESI⁺) M+H⁺: 338.

### 6-(1-(2-Hydroxyethyl)-4-phenyl-1H-pyrazol-3-yl)-2H-benzo[b][1,4]oxazin-3(4H)-one

6-(1-(2-Hydroxyethyl)-4-phenyl-1H-pyrazol-3-yl)-2H-benzo[b][1,4]oxazin-3(4H)-one was also isolated as a white solid (10 mg, 4%).

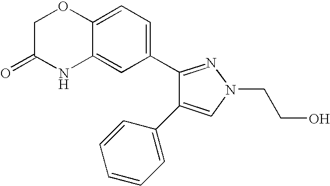

¹H-NMR (400 MHz, CDCl₃) 6: (brs, 1H), 7.72 (s, 1H), 7.22 (m, 2H), 7.16 (m, 3H), 7.03 (d, J=8.4 Hz, 1H), 6.93 (dd, J=8.4, 2.0 Hz, 1H), 6.81 (d, J=2.0 Hz, 1H), 4.66 (s, 2H), 4.11 (m, 2H), 4.00 (m, 2H), 3.64 (brs, 1H); LCMS (ESI⁺) M+H⁺: 336.

### Example 218

### 6-(1-(2,4,6-Trichlorophenyl)-3-(trifluoromethyl)-1H-pyrazol-5-yl)-2H-benzo[b][1,4]oxazin-3(4H)-one

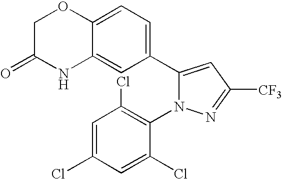

According to the method of Example 71 but in the absence of triethylamine, 4,4,4-trifluoro-1-(3-oxo-3,4-dihydro-2H-benzo[b][1,4]oxazin-6-yl)butane-1,3-dione (100 mg, 0.348 mmol) and 1-(2,4,6-trichlorophenyl)hydrazine (77.3 mg, 0.366 mmol) gave the title compound as an ivory powder (117 mg, 69%).

¹H-NMR (400 MHz, CDCl₃) δ: 8.36 (brs, 1H), 7.44 (s, 2H), 6.92 (d, J=8.4 Hz, 1H), 6.84 (dd, J=8.4, 2.0 Hz, 1H), 6.75 (s, 1H), 6.71 (d, J=2.0 Hz, 1H), 4.65 (s, 2H); LCMS (ESI⁺) M+H⁺: 464.

### Example 219

### 6-(1-(2,3-Dimethylphenyl)-3-(trifluoromethyl)-1H-pyrazol-5-yl)-2H-benzo[b][1,4]oxazin-3(4H)-one

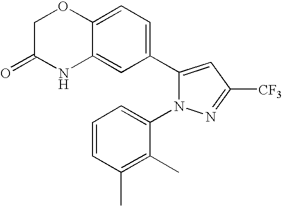

According to the method of Example 71, 4,4,4-trifluoro-1-(3-oxo-3,4-dihydro-2H-benzo[b][1,4]oxazin-6-yl)butane-1,3-dione (100 mg, 0.348 mmol) and 1-(2,3-dimethylphenyl)hydrazine hydrochloride (63.1 mg, 0.366 mmol) gave the title compound as pale orange crystals (48.0 mg, 36%) after recrystallization from ethanol/water.

¹H-NMR (400 MHz, CDCl₃) δ: 7.73 (brs, 1H), 7.26 (d, J=7.4 Hz, 1H), 7.16 (dd, J=7.8, 7.4 Hz, 1H), 7.11 (d, J=7.8 Hz, 1H), 6.87 (d, J=8.6 Hz, 1H), 6.79 (dd, J=8.6, 2.0 Hz, 1H), 6.75 (s, 1H), 6.54 (d, J=2.0 Hz, 1H), 4.61 (s, 2H), 2.30 (s, 3H), 1.83 (s, 3H); LCMS (ESI⁺) M+H⁺: 388.

### Example 220

### 6-(1-(3-Chlorophenyl)-3-(trifluoromethyl)-1H-pyrazol-5-yl)-2H-benzo[b][1,4]oxazin-3(4H)-one

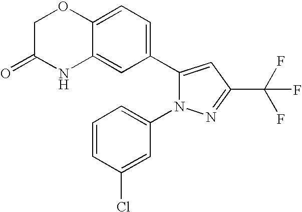

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(3-chlorophenyl)hydrazine hydrochloride (147 mg, 0.823 mmol) gave the title compound as an ivory solid (286 mg, 90%).

¹H-NMR (400 MHz, CDCl₃) δ: 8.07 (brs, 1H), 7.45 (t, J=2.0 Hz, 1H), 7.37 (ddd, J=8.2, 2.0, 1.2 Hz, 1H), 7.29 (dd, J=8.2, 7.8 Hz, 1H), 7.12 (ddd, J=7.8, 2.0, 1.2 Hz, 1H), 6.95 (d, J=8.4 Hz, 1H), 6.82 (dd, J=8.4, 2.0 Hz, 1H), 6.71 (s, 1H), 6.67 (d, J=2.0 Hz, 1H), 4.67 (s, 2H); LCMS (ESI⁺) M+H⁺: 394.

### Example 221

### 6-(1-(2,4-Dimethylphenyl)-3-(trifluoromethyl)-1H-pyrazol-5-yl)-2H-benzo[b][1,4]oxazin-3(4H)-one

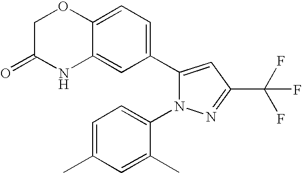

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(2,4-dimethylphenyl)hydrazine hydrochloride (142 mg, 0.823 mmol) gave, after flash chromatography on silica gel (10-30% EtOAc in petroleum ether), the title compound as a yellow solid (109 mg, 36%).

¹H-NMR (400 MHz, CDCl₃) δ: 7.57 (brs, 1H), 7.14 (d, J=8.6 Hz, 1H), 7.06 (m, 2H), 6.88 (d, J=8.4 Hz, 1H), 6.81 (dd, J=8.4, 1.8 Hz, 1H), 6.74 (s, 1H), 6.54 (d, J=1.8 Hz, 1H), 4.61 (s, 2H), 2.37 (s, 3H), 1.92 (s, 3H); LCMS (ESI⁺) M+H⁺: 388.

### Example 222

### 6-(3-(Trifluoromethyl)-1-(3-(trifluoromethyl)phenyl)-1H-pyrazol-5-yl)-2H-benzo[b][1,4]oxazin-3(4H)-one

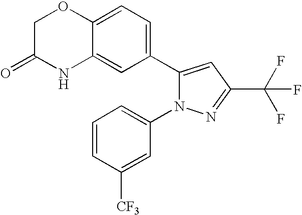

According to the method of Example 71 but in the absence of triethylamine, 4,4,4-trifluoro-1-(3-oxo-3,4-dihydro-2H-benzo[b][1,4]oxazin-6-yl)butane-1,3-dione (200 mg, 0.696 mmol) and 1-(3-(trifluoromethyl)phenyl)hydrazine (129 mg, 0.731 mmol) gave the title compound as pale tan crystals (186 mg, 60%) after recrystallization from ethanol/water.

¹H-NMR (400 MHz, CDCl₃) δ: 8.36 (brs, 1H), 7.67 (s, 1H), 7.64 (d, J=7.4 Hz, 1H), 7.49 (m, 2H), 6.95 (d, J=8.4 Hz, 1H), 6.80 (dd, J=8.4, 2.0 Hz, 1H), 6.74 (s, 1H), 6.68 (d, J=2.0 Hz, 1H), 4.65 (s, 2H); LCMS (ESI⁺) M+H⁺: 428.

### Example 223

### 6-(1-(3-Bromophenyl)-3-(trifluoromethyl)-1H-pyrazol-5-yl)-2H-benzo[b][1,4]oxazin-3(4H)-one

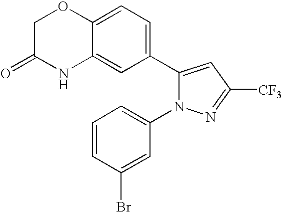

According to the method of Example 71, 4,4,4-trifluoro-1-(3-oxo-3,4-dihydro-2H-benzo[b][1,4]oxazin-6-yl)butane-1,3-dione (200 mg, 0.696 mmol) and 1-(3-bromophenyl)hydrazine hydrochloride (163 mg, 0.731 mmol) gave the title compound as an ivory solid (272 mg, 86%).

¹H-NMR (400 MHz, CDCl₃) δ: 7.84 (brs, 1H), 7.61 (t, J=2.0 Hz, 1H), 7.52 (dd, J=8.0, 1.5 Hz, 1H), 7.23 (t, J=8.0 Hz, 1H), 7.16 (d, J=8.0 Hz, 1H), 6.96 (d, J=8.4 Hz, 1H), 6.83 (dd, J=8.4, 2.0 Hz, 1H), 6.71 (s, 1H), 6.66 (d, J=2.0 Hz, 1H), 4.67 (s, 2H); LCMS (ESI⁺) M+H⁺: 440.

### Example 224

### 6-(1-(4-Fluoro-2,6-dimethylphenyl)-3-(trifluoromethyl)-1H-pyrazol-5-yl)-2H-benzo[b][1,4]oxazin-3(4H)-one

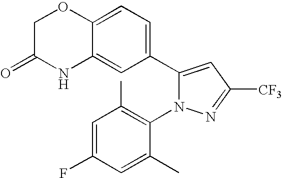

### 1-(4-Fluoro-2,6-dimethylphenyl)hydrazine

According to the method of Example 107, 4-fluoro-2,6-dimethylbenzenamine (1.6 g, 11.5 mmol) gave the title compound (420 mg, 24%).

### 6-(1-(4-Fluoro-2,6-dimethylphenyl)-3-(trifluoromethyl)-1H-pyrazol-5-yl)-2H-benzo[b][1,4]oxazin-3(4H)-one

According to the method of Example 71, 4,4,4-trifluoro-1-(3-oxo-3,4-dihydro-2H-benzo[b][1,4]oxazin-6-yl)butane-1,3-dione (197 mg, 0.688 mmol) and 1-(4-fluoro-2,6-dimethylphenyl)hydrazine (106 mg, 0.688 mmol) were reacted to give the title compound as a yellow solid (9.9 mg, 3%).

¹H-NMR (400 MHz, CDCl₃) δ: 9.14 (brs, 1H), 6.75-6.89 (m, 5H), 6.59 (s, 1H), 4.63 (s, 2H), 1.94 (s, 6H); LCMS (ESI⁻) M−H⁻: 404.

### Example 225

### 6-(1,3-Dimethyl-1H-pyrazol-5-yl)-2H-benzo[b][1,4]oxazin-3(4H)-one

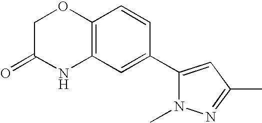

According to the method of Example 71, 1-(3-oxo-3,4-dihydro-2H-benzo[b][1,4]oxazin-6-yl)butane-1,3-dione (0.500 g, 2.144 mmol) and 1-methylhydrazine (0.1185 mL, 2.251 mmol) gave the title compound as a white solid (218 mg, 42%).

¹H-NMR (400 MHz, CDCl₃) δ: 8.14 (brs, 1H), 7.05 (d, J=8.2 Hz, 1H), 7.00 (dt, J=8.2 Hz, 1H), 6.82 (m, 1H), 6.04 (s, 1H), 4.67 (s, 2H), 3.79 (s, 3H), 2.29 (s, 3H); LCMS (ESI⁺): 244 M+H⁺

### Example 226

### 6-(1-(2,6-Dimethylphenyl)-3-(trifluoromethyl)-1H-pyrazol-5-yl)-2H-benzo[b][1,4]oxazin-3(4H)-one

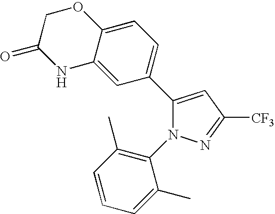

### 1-(2,6-Dimethylphenyl)hydrazine

According to the method of Example 107, 2,6-dimethylaniline (2.03 mL, 16.5 mmol) gave the title compound as a red-orange oil (1.54 g, 69%).

LCMS (ESI⁺) M+H⁺: 137.

### 6-(1-(2,6-Dimethylphenyl)-3-(trifluoromethyl)-1H-pyrazol-5-yl)-2H-benzo[b][1,4]oxazin-3(4H)-one

According to the method of Example 71 but in the absence of triethylamine, 4,4,4-trifluoro-1-(3-oxo-3,4-dihydro-2H-benzo[b][1,4]oxazin-6-yl)butane-1,3-dione (300 mg, 1.04 mmol) and 1-(2,6-dimethylphenyl)hydrazine (149 mg, 1.10 mmol) gave the title compound as a brown solid (191 mg, 44%).

¹H-NMR (400 MHz, CDCl₃) δ: 7.77 (brs, 1H), 7.28 (t, 1H), 7.13 (d, J=7.6 Hz, 2H), 6.87 (d, J=8.3 Hz, 1H), 6.80 (s, 1H), 6.79 (dd, J=13.2, 8.2 Hz, 1H), 6.50 (d, J=2.0 Hz, 1H), 4.61 (s, 2H), 1.95 (s, 6H); LCMS (APCI⁻) M−H⁻: 386.

### Example 227

### 6-(1-(2-Chloro-6-methylphenyl)-3-(trifluoromethyl)-1H-pyrazol-5-yl)-2H-benzo[b][1,4]oxazin-3(4H)-one

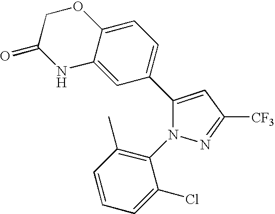

### 1-(2-Chloro-6-methylphenyl)hydrazine

According to the method of Example 107, 2-chloro-6-methylbenzenamine (2.00 g, 14.1 mmol) gave the title compound as a yellow-orange solid (842 mg, 30%).

LCMS (ESI⁺) M+H⁺: 157.

### 6-(1-(2-Chloro-6-methylphenyl)-3-(trifluoromethyl)-1H-pyrazol-5-yl)-2H-benzo[b][1,4]oxazin-3(4H)-one

According to the method of Example 71 but in the absence of triethylamine, 4,4,4-trifluoro-1-(3-oxo-3,4-dihydro-2H-benzo[b][1,4]oxazin-6-yl)butane-1,3-dione (300 mg, 1.04 mmol) and 1-(2-chloro-6-methylphenyl)hydrazine (172 mg, 1.10 mmol) gave the title compound as a tan solid (263 mg, 59%).

¹H-NMR (400 MHz, CDCl₃) δ: 7.78 (brs, 1H), 7.34 (s, 1H), 7.33 (q, J=7.2 Hz, 1H), 7.20 (m, 1H), 6.88 (d, J=8.4 Hz, 1H), 6.82 (dd, J=8.4, 2.0 Hz, 1H), 6.78 (s, 1H), 6.62 (d, J=2.0 Hz, 1H), 4.62 (s, 2H), 2.01 (s, 3H); LCMS (APCI⁻), M−H⁻: 406.

### Example 228

### 6-(1-(5-Chloro-2-fluorophenyl)-3-(trifluoromethyl)-1H-pyrazol-5-yl)-2H-benzo[b][1,4]oxazin-3(4H)-one

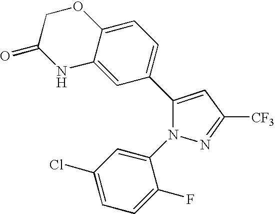

### 1-(5-Chloro-2-fluorophenyl)hydrazine

According to the method of Example 107, 5-chloro-2-fluorobenzenamine (2.00 g, 13.7 mmol) gave the title compound as a red-orange solid (857 mg, 31%. LCMS (ESI⁺) M+H⁺: 161.

### 6-(1-(5-Chloro-2-fluorophenyl)-3-(trifluoromethyl)-1H-pyrazol-5-yl)-2H-benzo[b][1,4]oxazin-3(4H)-one

According to the method of Example 71 but in the absence of triethylamine, 4,4,4-trifluoro-1-(3-oxo-3,4-dihydro-2H-benzo[b][1,4]oxazin-6-yl)butane-1,3-dione (300 mg, 1.04 mmol) and 1-(5-chloro-2-fluorophenyl)hydrazine (176 mg, 1.10 mmol) gave the title compound as a tan solid (245 mg, 52%).

¹H-NMR (400 MHz, CDCl₃) δ: 7.76 (brs, 1H), 7.60 (dd, J=6.2, 2.7 Hz, 1H), 7.41 (m, 1H), 7.06 (t, J=9.0 Hz, 1H), 6.93 (d, J=8.4 Hz, 1H), 6.80 (dd, J=8.4, 2.0 Hz, 1H), 6.73 (s, 1H), 6.68 (d, J=2.0 Hz, 1H), 4.66 (s, 2H); LCMS (APCI⁻) M−H⁻: 410.

### Example 229

### 6-(1-(4-Chloro-2-fluorophenyl)-3-(trifluoromethyl)-1H-pyrazol-5-yl)-2H-benzo[b][1,4]oxazin-3(4H)-one

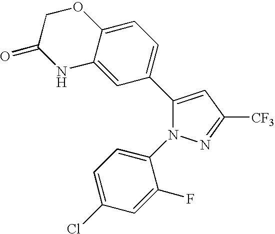

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(4-chloro-2-fluorophenyl)hydrazine hydrochloride (154 mg, 0.783 mmol) gave the title compound as a light beige solid (247 mg, 72%).

¹H-NMR (400 MHz, CDCl₃) δ: 7.74 (brs, 1H), 7.48 (t, J=8.2 Hz, 1H), 7.28 (m, 1H), 7.16 (dd, J=9.5, 2.2 Hz, 1H), 6.92 (d, J=8.4 Hz, 1H), 6.78 (dd, J=8.4, 2.0 Hz, 1H), 6.72 (s, 1H), 6.67 (d, J=2.0 Hz, 1H), 4.65 (s, 2H); LCMS (APCI⁻), M−H⁻: 410.

### Example 230

### 6-(1-(2,6-Difluorophenyl)-3-(trifluoromethyl)-1H-pyrazol-5-yl)-2H-benzo[b][1,4]oxazin-3(4H)-one

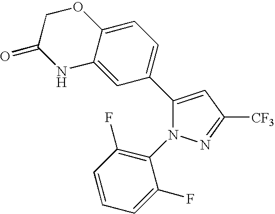

According to the method of Example 71 but in the absence of triethylamine, 4,4,4-trifluoro-1-(3-oxo-3,4-dihydro-2H-benzo[b][1,4]oxazin-6-yl)butane-1,3-dione (225 mg, 0.783 mmol) and 1-(2,6-difluorophenyl)hydrazine (119 mg, 0.823 mmol) gave the title compound as a tan solid (145 mg, 46%).

¹H-NMR (400 MHz, CDCl₃) δ: 8.03 (brs, 1H), 7.44 (m, 1H), 7.02 (dd, J=8.7, 1.6 Hz, 2H), 6.90 (d, J=8.4 Hz, 1H), 6.84 (dd, J=8.4, 1.9 Hz, 1H), 6.75 (s, 1H), 6.72 (d, J=1.9 Hz, 1H), 4.64 (s, 2H); LCMS (APCI⁻) M−H⁻: 394.

### Example 231

### 6-(1-(2,6-Dichlorophenyl)-3-(trifluoromethyl)-1H-pyrazol-5-yl)-2H-benzo[b][1,4]oxazin-3(4H)-one

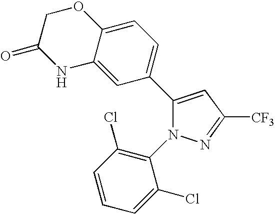

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(2,6-dichlorophenyl)hydrazine hydrochloride (176 mg, 0.823 mmol) gave the title compound as a yellow solid (241 mg, 68%).

¹H-NMR (400 MHz, CDCl₃) δ: 7.70 (brs, 1H), 7.41 (m, 3H), 6.90 (d, J=8.4 Hz, 1H), 6.87 (dd, J=8.4, 1.7 Hz, 1H), 6.75 (s, 1H), 6.69 (d, J=1.7 Hz, 1H), 4.63 (s, 2H); LCMS (APCI⁻) M−H⁻: 426.

### Example 232

### 6-(1-(3-Chloro-4-fluorophenyl)-3-(trifluoromethyl)-1H-pyrazol-5-yl)-2H-benzo[b][1,4]oxazin-3(4H)-one

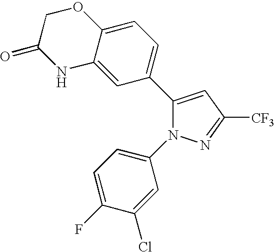

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(3-chloro-4-fluorophenyl)hydrazine hydrochloride (162 mg, 0.823 mmol) gave the title compound as a pale orange solid (262 mg, 77%).

¹H-NMR (400 MHz, CDCl₃) δ: 7.18 (brs, 1H), 7.52 (m, 1H), 7.13 (m, 2H), 6.97 (d, J=8.4 Hz, 1H), 6.81 (dd, J=8.4, 2.0 Hz, 1H), 6.71 (s, 1H), 6.67 (d, J=2.0 Hz, 1H), 4.67 (s, 2H); LCMS (APCI⁻) M−H⁻: 410.

### Example 233

### 6-(1-(3,5-Difluorophenyl)-3-(trifluoromethyl)-1H-pyrazol-5-yl)-2H-benzo[b][1,4]oxazin-3(4H)-one

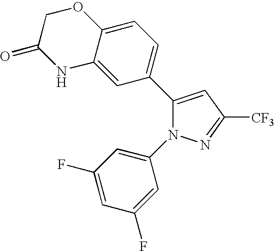

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(3,5-difluorophenyl)hydrazine hydrochloride (149 mg, 0.823 mmol) gave the title compound as an ivory solid (96.0 mg, 29%) after recrystallization from ethanol/water.

¹H-NMR (400 MHz, CDCl₃) δ: 7.89 (brs, 1H), 6.99 (d, J=7.9 Hz, 1H), 6.90 (dd, J=7.9, 2.1 Hz, 2H), 6.89-6.81 (m, 2H), 6.70 (s, 1H), 6.69 (d, J=2.1 Hz, 1H), 4.69 (s, 2H); LCMS (APCI⁻) M−H⁻: 394.

### Example 234

### 6-(1-(3-Methoxyphenyl)-3-(trifluoromethyl)-1H-pyrazol-5-yl)-2H-benzo[b][1,4]oxazin-3(4H)-one

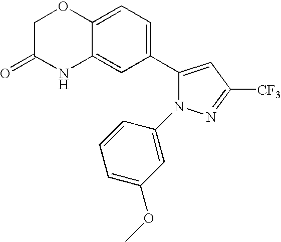

### tert-Butyl 1-(3-methoxyphenyl)hydrazinecarboxylate

A mixture of 1-iodo-3-methoxybenzene (1.02 mL, 8.55 mmol), t-butylcarbazate (1.36 g, 10.3 mmol), Cs₂CO₃ (3.90 g, 12.0 mmol), 1,10-phenanthroline (308 mg, 1.71 mmol), and Cu(I)I (81 mg, 0.43 mmol) in dry DMF (8.6 mL) under nitrogen was heated at 80° C. for 20 hr. The cooled reaction mixture was passed through silica gel (EtOAc) and purified by flash chromatography on silica gel (10-25% EtOAc in hexanes) to give the title compound as a yellow oil (1.64 g, 80%).

¹H-NMR (400 MHz, CDCl₃) δ: 7.20 (m, 1H), 7.08 (m, 2H), 6.67 (m, 1H), 4.41 (brs, 2H), 3.80 (s, 3H), 1.51 (s, 9H).

### (3-Methoxyphenyl)hydrazine

To a stirred solution of tert-butyl 1-(3-methoxyphenyl)hydrazinecarboxylate (1.0 g, 4.2 mmol) in DCM (10 mL) at room temperature was added TFA (4 mL) and stirring was continued for 3 hr. The residue was dissolved in water and extracted with ether. The aqueous layer basified with aqueous NaOH solution, extracted twice with ether, and the organic layer was washed with water and brine, dried (MgSO₄) and concentrated in vacuo to give the title compound as a thick yellow liquid (540 mg, 93%).

LCMS (APCI⁺) M+H⁺: 139.

### 6-(1-(3-Methoxyphenyl)-3-(trifluoromethyl)-1H-pyrazol-5-yl)-2H-benzo[b][1,4]oxazin-3(4H)-one

According to the method of Example 71 but in the absence of triethylamine, 6-(4,4,4-trifluoro-3-oxobutanoyl)-2H-benzo[b][1,4]oxazin-3(4H)-one (1122 mg, 3.91 mmol) and 1-(3-methoxyphenyl)hydrazine (540 mg, 3.91 mmol) were reacted to give the title compound as a tan solid (1.2 g, 79%).

¹H-NMR (400 MHz, CDCl₃) δ: 9.48 (brs, 1H), 7.24 (m, 1H), 6.92 (m, 3H), 6.81 (m, 2H), 6.75 (d, J=1.8 Hz, 1H), 6.69 (s, 1H), 4.63 (s, 2H), 3.77 (s, 3H); LCMS (ESI⁻) M−H⁻: 388.

### Example 235

### 6-(1-(5-Fluoro-2-methylphenyl)-3-(trifluoromethyl)-1H-pyrazol-5-yl)-2H-benzo[b][1,4]oxazin-3(4H)-one

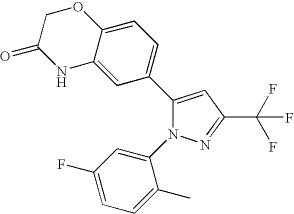

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(5-fluoro-2-methylphenyl)hydrazine hydrochloride (145 mg, 0.823 mmol) gave the title compound as a pale yellow-orange solid (37.0 mg, 11%).

¹H-NMR (400 MHz, CDCl₃) δ: 8.40 (brs, 1H), 7.24 (m, 1H), 7.10 (td, J=8.2, 2.5 Hz, 1H), 7.04 (dd, J=8.2, 2.5 Hz, 1H), 6.89 (d, J=8.3 Hz, 1H), 6.77 (dd, J=8.3, 2.0 Hz, 1H), 6.75 (s, 1H), 6.62 (d, J=2.0 Hz, 1H), 4.64 (s, 2H), 1.93 (s, 3H); LCMS (APCI⁻) M−H⁻: 390.

### Example 236

### 6-(1-(Pyridin-3-yl)-3-(trifluoromethyl)-1H-pyrazol-5-yl)-2H-benzo[b][1,4]oxazin-3(4H)-one

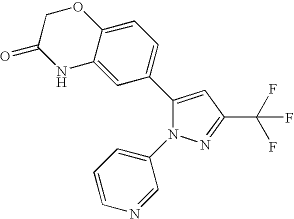

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(pyridin-3-yl)hydrazine hydrochloride (266 mg, 0.823 mmol) gave the title compound as an ivory solid (184 mg, 29%).

¹H-NMR (400 MHz, CDCl₃) δ: 7 8.62 (brs, 1H), 8.55 (brs, 1H), 8.35 (brs, 1H), 7.78 (d, J=8.0 Hz, 1H), 7.40 (dd, J=8.0, 3.5 Hz, 1H), 6.96 (d, J=8.4 Hz, 1H), 6.82 (dd, J=8.4, 2.0 Hz, 1H), 6.75 (s, 1H), 6.69 (d, J=2.0 Hz, 1H), 4.66 (s, 2H); LCMS (ESI⁻) M−H⁻: 359.

### Example 237

### 6-(1-(2,5-Difluorophenyl)-3-(trifluoromethyl)-1H-pyrazol-5-yl)-2H-benzo[b][1,4]oxazin-3(4H)-one

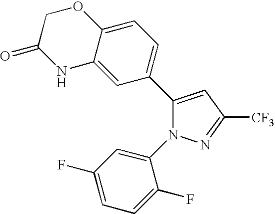

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(2,5-difluorophenyl)hydrazine hydrochloride (149 mg, 0.823 mmol) gave, after flash chromatography on silica gel (10-30% EtOAc in petroleum ether), the title compound as a yellow solid (37.0 mg, 11%).

¹H-NMR (400 MHz, CDCl₃) δ: 7.83 (brs, 1H), 7.30 (m, 1H), 7.16 (m, 1H), 7.09 (m, 1H), 6.92 (d, J=8.4 Hz, 1H), 6.80 (dd, J=8.4, 2.0 Hz, 1H), 6.73 (s, 1H), 6.68 (d, J=2.0 Hz, 1H), 4.65 (s, 2H); LCMS (ESI⁻) M−H⁻: 394.

### Example 238

### 6-(1-(4-Methylphenethyl)-3-(trifluoromethyl)-1H-pyrazol-5-yl)-2H-benzo[b][1,4]oxazin-3(4H)-one

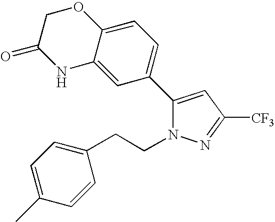

### (4-Methylphenethyl)hydrazine

According to the method of Fishwick, C. W. G., et al. (Tetrahedron, 2003, 59, 4451-4468), 2-p-tolylacetaldehyde (2.00 g, 7.45 mmol) and t-butylcarbazate (985 mg, 7.45 mmol) gave the title compound as a red-orange solid (408 mg, 31%).

### 6-(1-(4-Methylphenethyl)-3-(trifluoromethyl)-1H-pyrazol-5-yl)-2H-benzo[b][1,4]oxazin-3(4H)-one

According to the method of Example 71 but in the absence of triethylamine, 4,4,4-trifluoro-1-(3-oxo-3,4-dihydro-2H-benzo[b][1,4]oxazin-6-yl)butane-1,3-dione (225 mg, 0.783 mmol) and (4-methylphenethyl)hydrazine (124 mg, 0.823 mmol) gave, after flash chromatography on silica gel (10-30% EtOAc in petroleum ether), the title compound as an ivory solid (79.0 mg, 24%).

¹H-NMR (400 MHz, CDCl₃) δ: 7.70 (brs, 1H), 7.07 (d, J=7.8 Hz, 2H), 6.95 (d, J=8.3 Hz, 1H), 6.82 (d, J=7.8 Hz, 2H), 6.67 (dd, J=8.3, 2.0 Hz, 1H), 6.39 (s, 1H), 5.96 (d, J=2.0 Hz, 1H), 4.65 (s, 2H), 4.24 (t, J=6.8 Hz, 2H), 3.14 (t, J=6.8 Hz, 2H), 2.34 (s, 3H); LCMS (ESI⁻) M−H⁻: 400.

### Example 239

### 6-(1-(4-Fluoro-2-methylphenyl)-3-(trifluoromethyl)-1H-pyrazol-5-yl)-5-methyl-2H-benzo[b][1,4]oxazin-3(4H)-one

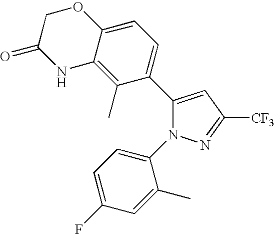

### 6-Acetyl-5-methyl-2H-benzo[b][1,4]oxazin-3(4H)-one

To a solution of 5-methyl-2H-benzo[b][1,4]oxazin-3(4H)-one (2.00 g, 12.26 mmol) and acetyl chloride (1.74 mL, 24.51 mmol) in CS₂ was added AlCl₃ (4.09 g, 30.64 mmol) slowly under gas evolution. The reaction mixture was stirred for 2 days at room temperature with a reflux condenser attached. After reaction completion was observed by LCMS, the mixture was poured onto ice and the whole mixture was stirred to quench excess AlCl₃. The slurry was diluted with EtOAc and the organic layer was separated. The aqueous layer was extracted twice with EtOAc, and the organic layer was washed with brine, dried (Na₂SO₄) and concentrated in vacuo. To the residue was added a minimal amount of DCM, and the mixture was sonicated and filtered. This treatment was repeated a second time, and the two crops were combined to give the title compound (2.00 g, 80%).

LCMS (ESI⁻) M−H⁻: 204.

### 4,4,4-Trifluoro-1-(5-methyl-3-oxo-3,4-dihydro-2H-benzo[b][1,4]oxazin-6-yl)butane-1,3-dione

To a 100 mL flask were added NaH (780 mg, 19.49 mmol) and THF (25 mL). To the stirring suspension were added ethyl 2,2,2-trifluoroacetate (2.33 mL, 19.49 mmol) and then 6-acetyl-5-methyl-2H-benzo[b][1,4]oxazin-3(4H)-one (1.00 g, 4.87 mmol). After gas evolution minimized, EtOH (0.5 mL) was added followed by dibenzo-18-crown-6 (28 mg, 0.08 mmol). The resulting light-brown solution was stirred at 65° C. overnight. The reaction mixture was cooled to room temperature and partitioned between 10% H₂SO₄ (200 mL) and EtOAc (200 mL). The organic layer was washed with water, saturated NaHCO₃, water and brine, dried (Na₂SO₄), and concentrated in vacuo. To the residue was added ether and the suspension was sonicated and filtered to give the title compound as an off-white solid (1.10 g, 75%).

LCMS (ESI⁻) M−H⁻: 300.

### 6-(1-(4-Fluoro-2-methylphenyl)-3-(trifluoromethyl)-1H-pyrazol-5-yl)-5-methyl-2H-benzo[b][1,4]oxazin-3(4H)-one

According to the method of Example 71, 4,4,4-trifluoro-1-(5-methyl-3-oxo-3,4-dihydro-2H-benzo[b][1,4]oxazin-6-yl)butane-1,3-dione (250 mg, 0.83 mmol) and 1-(4-fluoro-2-methylphenyl)hydrazine (116 mg, 0.83 mmol), in the absence of triethylamine, gave the title compound as an off-white solid (53.0 mg, 16%).

¹H-NMR (400 MHz, CDCl₃) δ: 10.27 (s, 1H), 7.34 (dd, J=9.0, 5.5 Hz, 1H), 7.22 (dd, J=9.8, 2.7 Hz, 1H), 7.08 (td, J=8.2, 2.7 Hz, 1H), 7.04 (s, 1H), 6.77 (d, J=8.6 Hz, 1H), 6.74 (d, J=8.2 Hz, 1H), 4.53 (s, 2H), 2.07 (s, 3H), 1.98 (s, 3H); LCMS (ESI⁻) M−H⁻: 404.

### Example 240

### 8-Chloro-6-(1-(4-fluorophenyl)-3-(trifluoromethyl)-1H-pyrazol-5-yl)-2H-benzo[b][1,4]oxazin-3(4H)-one

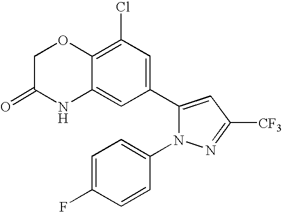

According to the method of Example 71, 1-(4-fluorophenyl)hydrazine hydrochloride (66 mg, 0.41 mmol), and 8-chloro-4,4,4-trifluoro-1-(3-oxo-3,4-dihydro-2H-benzo[b][1,4]oxazin-6-yl)butane-1,3-dione (131 mg, 0.41 mmol) gave the title compound after trituration with ether (34.0 mg, 19%).

¹H-NMR (400 MHz, DMSO-d₆) δ: 10.95 (s, 1H), 7.46 (m, 2H), 7.35 (m, 2H), 7.20 (s, 1H), 7.09 (s, 1H), 6.65 (s, 1H), 4.73 (s, 2H); LCMS (ESI⁻) M−H⁻: 410.

### Example 241

### 5-(1-(4-Fluorophenyl)-3-(trifluoromethyl)-1H-pyrazol-5-yl)benzo[d]oxazol-2(3H)-one

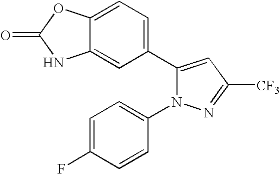

### 4,4,4-Trifluoro-1-(4-hydroxy-3-nitrophenyl)butane-1,3-dione

According to the method of Example 71, ethyl 2,2,2-trifluoroacetate (13.17 mL, 110.4 mmol) and 1-(4-hydroxy-3-nitrophenyl)ethanone (5.000 g, 27.60 mmol) were reacted to give the title compound as a brown oil (5.90 g, 77%).

LCMS (ESI⁺) M+H⁺: 278.

### 4-(1-(4-Fluorophenyl)-3-(trifluoromethyl)-1H-pyrazol-5-yl)-2-nitrophenol

According to the method of Example 71, 1-(4-fluorophenyl)hydrazine hydrochloride (1.163 g, 7.151 mmol), and 4,4,4-trifluoro-3-hydroxy-1-(4-hydroxy-3-nitrophenyl)but-2-en-1-one (1.982 g, 7.151 mmol) were reacted to give the title compound (2.13 g, 81%).

LCMS (ESI⁻) M−H⁻: 366.

### 2-Amino-4-(1-(4-fluorophenyl)-3-(trifluoromethyl)-1H-pyrazol-5-yl)phenol

To a stirred solution of 4-(1-(4-fluorophenyl)-3-(trifluoromethyl)-1H-pyrazol-5-yl)-2-nitrophenol (2.75 g, 7.49 mmol) in acetic acid (100 mL) was slowly added zinc dust (2.45 g, 37.44 mmol) and the reaction mixture was heated at 80° C. overnight. The reaction mixture was filtered and the filtrate concentrated was in vacuo to afford the title compound as a brown oil (2.10 g, 83%).

LCMS (ESI⁺) M+H⁺: 338.

### 5-(1-(4-Fluorophenyl)-3-(trifluoromethyl)-1H-pyrazol-5-yl)benzo[d]oxazol-2(3H)-one

A solution of 2-amino-4-(1-(4-fluorophenyl)-3-(trifluoromethyl)-1H-pyrazol-5-yl)phenol (0.159 g, 0.4714 mmol), CDI (0.1529 g, 0.9429 mmol), and TEA (0.1971 mL, 1.414 mmol) in DCE was heated at 80° C. for 1 hr. The reaction mixture was poured into water, extracted with DCM and the organic layer was washed with brine, dried (Na₂SO₄) and concentrated in vacuo. Flash chromatography of the residue on silica gel gave the title compound as a white solid (29.2 mg, 17%).

¹H-NMR (400 MHz, DMSO-d₆) δ: 11.77 (s, 1H), 7.45 (m, 2H), 7.32 (t, J=7.2 Hz, 3H), 7.20 (s, 1H), 6.99 (m, 2H); (ESI⁻) M−H⁻: 362.

### Example 242

### 6-(1-(4-Fluoro-2-methylphenyl)-3-methyl-1H-pyrazol-5-yl)-2H-benzo[b][1,4]oxazin-3(4H)-one

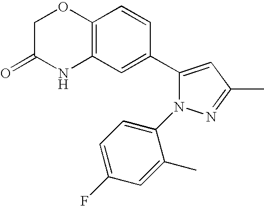

According to the method of Example 71, 1-(3-oxo-3,4-dihydro-2H-benzo[b][1,4]oxazin-6-yl)butane-1,3-dione (100 mg, 0.4288 mmol, Example 97) and 1-(4-fluoro-2-methylphenyl)hydrazine hydrochloride (79.52 mg, 0.4502 mmol) were reacted to give the title compound as a tan solid (84 mg, 58%).

¹H-NMR (400 MHz, CDCl₃) δ: 8.33 (brs, 1H), 7.23 (m, 1H), 6.84 (d, J=8.2 Hz, 2H), 6.84 (d, J=8.2 Hz, 1H), 6.74 (dd, J=8.2, 2.0 Hz, 1H), 6.57 (d, J=2.0 Hz, 1H), 6.29 (s, 1H), 4.62 (s, 2H), 2.36 (s, 3H), 1.95 (s, 3H): LCMS (ESI⁺) (M+H⁺): 338

### Example 243

### Methyl 3-(5-(3-oxo-3,4-dihydro-2H-benzo[b][1,4]oxazin-6-yl)-3-(trifluoromethyl)-1H-pyrazol-1-yl)thiophene-2-carboxylate

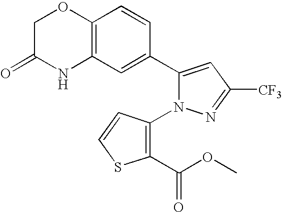

According to the method of Example 71, methyl 3-hydrazinylthiophene-2-carboxylate (0.180 g, 1.04 mmol) and 6-(4,4,4-trifluoro-3-oxobutanoyl)-2H-benzo[b][1,4]oxazin-3(4H)-one (0.300 g, 1.04 mmol) were reacted in the absence of triethylamine to give the title compound (15.8 mg, 4%).

¹H-NMR (400 MHz, DMSO-d₆) δ: 10.8 (s, 1H), 8.07 (d, J=5.5 Hz, 1H), 7.38 (d, J=5.5 Hz, 1H), 7.09 (s, 1H), 6.92 (d, J=8.2 Hz, 1H), 6.91 (s, 1H), 6.80 (dd, J=8.2, 2.0 Hz, 1H), 6.76 (d, J=2.0 Hz, 1H), 4.59 (s, 2H), 3.61 (s, 3H); (ESI⁻) M−H⁻: 422.

### Example 244

### 6-(1-(4-Fluorophenyl)-3-(trifluoromethyl)-1H-pyrazol-5-yl)-2H-benzo[b][1,4]thiazin-3(4H)-one

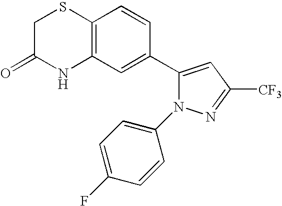

### 6-Acetyl-2H-benzo[b][1,4]thiazin-3(4H)-one

According to the method of Example 239, 2H-1,4-benzothiazin-3(4H)-one (5.00 g, 30.26 mmol) and acetyl chloride (3.23 mL, 45.40 mmol) were reacted to give the title compound (4.35 g, 69%).

LCMS (ESI⁻) M−H⁻: 206.

### 6-(4,4,4-Trifluoro-3-oxobutanoyl)-2H-benzo[b][1,4]thiazin-3(4H)-one

According to the method of Example 71, ethyl 2,2,2-trifluoroacetate (2.74 g, 19.30 mmol), and 6-acetyl-2H-benzo[b][1,4]thiazin-3(4H)-one (1.00 g, 4.83 mmol), were reacted to give the title compound as a yellow solid (840 mg, 57%).

LCMS (ESI⁻) M−H⁻: 302.

### 6-(1-(4-Fluorophenyl)-3-(trifluoromethyl)-1H-pyrazol-5-yl)-2H-benzo[b][1,4]thiazin-3(4H)-one

According to the method of Example 71, 1-(4-fluorophenyl)hydrazine hydrochloride (107 mg, 0.66 mmol) and 6-(4,4,4-trifluoro-3-oxobutanoyl)-2H-benzo[b][1,4]thiazin-3(4H)-one (200 mg, 0.66 mmol) were reacted in the absence of triethylamine to give the title compound (209 mg, 81%).

¹H-NMR (400 MHz, DMSO-d₆) δ: 10.66 (s, 1H), 7.46-7.42 (m, 2H), 7.35-7.31 (m, 3H), 6.89 (d, J=2.0 Hz, 1H), 6.85 (dd, J=8.2, 2.0 Hz, 1H), 3.50 (s, 3H); LCMS (ESI⁻) M−H⁻: 392.

### Example 245

### 6-(1-(4-Fluoro-2-methylphenyl)-3-(trifluoromethyl)-1H-pyrazol-5-yl)-2H-benzo[b][1,4]thiazin-3(4H)-one

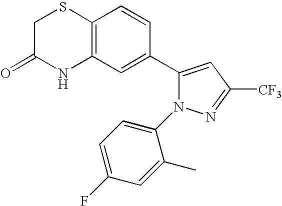

According to the method of Example 71, 1-(4-fluoro-2-methylphenyl)hydrazine hydrochloride (0.116 g, 0.660 mmol) and 6-(4,4,4-trifluoro-3-oxobutanoyl)-2H-benzo[b][1,4]thiazin-3(4H)-one (200 mg, 0.660 mmol) were reacted in the absence of triethylamine to give the title compound (65 mg, 24%).

¹H-NMR (400 MHz, DMSO-d₆) δ: 10.65 (s, 1H), 7.43 (s, J=8.6, 5.5 Hz, 1H), 7.31-7.27 (m, 2H), 7.20-7.17 (m, 2H), 6.85-6.82 (m, 2H), 3.48 (s, 2H), 1.92 (s, 3H); LCMS (ESI⁻) M−H⁻: 406.

### Example 246

### 3-(5-(3-Oxo-3,4-dihydro-2H-benzo[b][1,4]oxazin-6-yl)-3-(trifluoromethyl)-1H-pyrazol-1-yl)benzonitrile

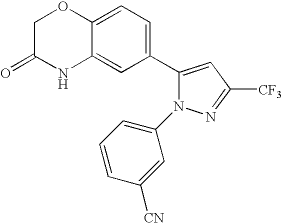

A mixture of 6-(1-(3-bromophenyl)-3-(trifluoromethyl)-1H-pyrazol-5-yl)-2H-benzo[b][1,4]oxazin-3(4H)-one (200 mg, 0.456 mmol), zinc cyanide (33.5 mg, 0.285 mmol) and tetrakis(triphenylphosphine)palladium(0) (33.0 mg, 0.0285 mmol) in degassed DMF (0.6 mL) was heated to 80° C. for 12 hr. The reaction mixture was diluted with toluene (5 mL), washed with NH₄OH (2N, 2×5 mL) and brine (5 mL). The organic layer was dried (Na₂SO₄), filtered and concentrated to give the title compound as an ivory solid (95.0 mg, 86%) after purification by flash chromatography on silica gel (10-30% EtOAc in petroleum ether).

¹H-NMR (400 MHz, CDCl₃) δ: 7.79 (brs, 1H), 7.68 (m, 2H), 7.58 (ddd, J=8.2, 1.6, 1.2 Hz, 1H), 7.53 (t, J=8.0 Hz, 1H), 6.97 (d, J=8.2 Hz, 1H), 6.78 (dd, J=8.2, 2.0 Hz, 1H), 6.73 (s, 1H), 6.67 (d, J=2.0 Hz, 1H), 4.69 (s, 2H);

LCMS (ESI⁺) M+H⁺: 385.

### Example 247

### 2-(5-(3-Oxo-3,4-dihydro-2H-benzo[b][1,4]oxazin-6-yl)-3-(trifluoromethyl)-1H-pyrazol-1-yl)benzonitrile

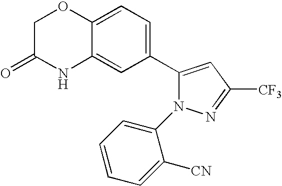

### 6-(1-(2-Bromophenyl)-3-(trifluoromethyl)-1H-pyrazol-5-yl)-2H-benzo[b][1,4]oxazin-3(4H)-one

According to the method of Example 71, 4,4,4-trifluoro-1-(3-oxo-3,4-dihydro-2H-benzo[b][1,4]oxazin-6-yl)butane-1,3-dione (225 mg, 0.783 mmol) and 1-(2-bromophenyl)hydrazine hydrochloride (184 mg, 0.823 mmol) gave the title compound as a pale yellow solid (285 mg, 83%).

LCMS (ESI⁺) M+H⁺: 440.

### 2-(5-(3-Oxo-3,4-dihydro-2H-benzo[b][1,4]oxazin-6-yl)-3-(trifluoromethyl)-1H-pyrazol-1-yl)benzonitrile

According to the method of Example 246, 6-(1-(2-bromophenyl)-3-(trifluoromethyl)-1H-pyrazol-5-yl)-2H-benzo[b][1,4]oxazin-3(4H)-one (205 mg, 0.468 mmol) and zinc cyanide (82.4 mg, 0.702 mmol) gave the title compound as a foamy yellow solid (28 mg, 16%) after purification by flash chromatography on silica gel (10-30% EtOAc in petroleum ether).

¹H-NMR (400 MHz, CDCl₃) δ: 7.73 (m, 2H), 7.57 (m, 2H), 7.53 (brs, 1H), 6.89 (d, J=8.5 Hz, 1H), 6.78 (s, 1H), 6.71 (dd, J=8.5, 2.0 Hz, 1H), 6.68 (d, J=2.0 Hz, 1H), 4.64 (s, 2H); (ESI⁻) M−H⁻: 383.

### Example 248

### 6-(1-(4-Fluoro-2-methylphenyl)-3-(trifluoromethyl)-1H-pyrazol-5-yl)-8-methoxy-2H-benzo[b][1,4]oxazin-3(4H)-one

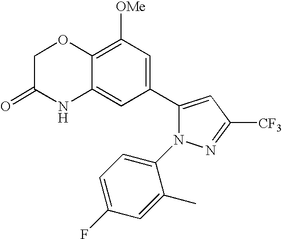

### 4-Bromo-2-methoxy-6-nitrophenol

According to the method described by Learmonth, D. A., et al. (JMC, 2002, 45, 685-695), 4-bromo-2-methoxyphenol (5.00 g, 24.6 mmol) and 70% nitric acid (1.71 g, 27.1 mmol) in acetic acid (62 mL) gave the title compound as an orange solid (3.87 g, 56%).

¹H-NMR (400 MHz, CDCl₃) δ: 10.7 (s, 1H), 7.86 (d, J=2.0 Hz, 1H), 7.21 (d, J=2.0 Hz, 1H), 3.95 (s, 3H); LCMS (ESI⁻) M−H⁻: 246, 248.

### Methyl 2-(4-bromo-2-methoxy-6-nitrophenoxy)acetate

According to the method of Example 108, 4-bromo-2-methoxy-6-nitrophenol (3.00 g, 12.1 mmol) and methyl 2-bromoacetate (1.17 mL, 12.3 mmol) were reacted at 50° C. to give the title compound as a light brown solid (3.29 g, 81%).

¹H-NMR (400 MHz, CDCl₃) δ: 7.51 (d, J=2.0 Hz, 1H), 7.22 (d, J=2.0 Hz, 1H), 4.75 (s, 2H), 3.91 (s, 3H), 3.79 (s, 3H).

### 6-Bromo-8-methoxy-2H-benzo[b][1,4]oxazin-3(4H)-one

According to the method of Example 108, methyl 2-(4-bromo-2-methoxy-6-nitrophenoxy)acetate (3.00 g, 9.37 mmol) gave the title compound as a brown solid (2.23 g, 83%).

¹H-NMR (400 MHz, CDCl₃) δ: 8.44 (brs, 1H), 6.76 (d, J=2.0 Hz, 1H), 6.63 (d, J=2.0 Hz, 1H), 4.66 (s, 2H), 3.88 (s, 3H); LCMS (ESI⁻), M−H⁻: 256, 258.

### 6-Acetyl-8-methoxy-2H-benzo[b][1,4]oxazin-3(4H)-one

According to the method of Example 108, 6-bromo-8-methoxy-2H-benzo[b][1,4]oxazin-3(4H)-one (2.00 g, 7.75 mmol) and 1-vinyloxy)butane (3.31 mL, 25.6 mmol) were reacted to give the title compound as a yellow solid (258 mg, 15%).

¹H-NMR (400 MHz, CDCl₃) δ: 7.83 (brs, 1H), 7.29 (d, J=2.0 Hz, 1H), 7.07 (d, J=2.0 Hz, 1H), 4.75 (s, 2H), 3.96 (s, 3H), 2.57 (s, 3H); LCMS (ESI⁻) M−H⁻: 220.

### 4,4,4-Trifluoro-1-(8-methoxy-3-oxo-3,4-dihydro-2H-benzo[b][1,4]oxazin-6-yl)butane-1,3-dione

According to the method in Example 71, ethyl 2,2,2-trifluoroacetate (0.442 mL, 3.71 mmol) and 6-acetyl-8-methoxy-2H-benzo[b][1,4]oxazin-3(4H)-one (205 mg, 0.927 mmol) gave the title compound as an orange solid (47.0 mg, 16%) after trituration from ether.

¹H-NMR (400 MHz, CDCl₃) δ: 7.83 (brs, 1H), 7.23 (d, J=2.0 Hz, 1H), 7.07 (d, J=2.0 Hz, 1H), 6.47 (s, 2H), 4.78 (s, 2H), 3.99 (s, 3H); LCMS (ESI⁻) M−H⁻: 316.

### 6-(1-(4-Fluoro-2-methylphenyl)-3-(trifluoromethyl)-1H-pyrazol-5-yl)-8-methoxy-2H-benzo[b][1,4]oxazin-3(4H)-one

According to the method in Example 71, 4,4,4-trifluoro-1-(8-methoxy-3-oxo-3,4-dihydro-2H-benzo[b][1,4]oxazin-6-yl)butane-1,3-dione (40.0 mg, 0.126 mmol) and 1-(4-fluoro-2-methylphenyl)hydrazine hydrochloride (23.4 mg, 0.132 mmol) gave the title compound as a golden yellow solid (24.0 mg, 44%).

¹H-NMR (400 MHz, CDCl₃) δ: 8.65 (brs, 1H), 7.30 (dd, J=9.4, 5.1 Hz, 1H), 7.00 (m, 2H), 6.78 (s, 1H), 6.34 (d, J=2.0 Hz, 1H), 6.32 (d, J=2.0 Hz, 1H), 4.67 (s, 2H), 3.66 (s, 3H), 1.97 (s, 3H); LCMS (ESI⁻) M−H⁻: 420.

### Example 249

### 8-Bromo-6-(1-(4-fluoro-2-methylphenyl)-3-(trifluoromethyl)-1H-pyrazol-5-yl)-2H-benzo[b][1,4]oxazin-3(4H)-one

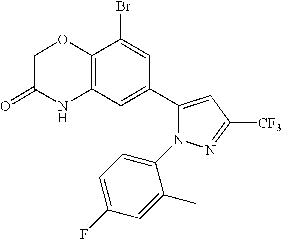

### Ethyl 3-bromo-4-hydroxy-5-nitrobenzoate

To a stirred Solution of ethyl 4-hydroxy-3-nitrobenzoate (20.00 g, 94.71 mmol) in acetic acid (189.4 mL, 94.71 mmol) was added bromine (9.70 mL, 189.4 mmol) and the resulting solution was stirred overnight at room temperature. The reaction mixture was poured into water and the precipitated yellow solid was collected by vacuum filtration and dried to give the title compound (26.0 g, 95%).

LCMS (ESI⁻) M−H⁻: 288, 290.

### Ethyl 3-bromo-4-(2-methoxy-2-oxoethoxy)-5-nitrobenzoate

According to the method of Example 108, ethyl 3-bromo-4-hydroxy-5-nitrobenzoate (40.00 g, 137.9 mmol) and methyl 2-bromoacetate (23.35 mL, 275.8 mmol) were reacted to give the title compound as a red oil (49.0 g, 98%).

### Ethyl 8-bromo-3-oxo-3,4-dihydro-2H-benzo[b][1,4]oxazine-6-carboxylate

According to the method of Example 108, ethyl 3-bromo-4-(2-methoxy-2-oxoethoxy)-5-nitrobenzoate (50.0 g, 138.1 mmol) and zinc dust (22.57 g, 345.2 mmol) were reacted to give the title compound (15.0 g, 36%).

¹H-NMR (400 MHz, DMSO-d₆) δ: 11.02 (brs, 1H), 7.70 (d, J=2.0 Hz, 1H), 7.49 (d, J=2.0 Hz, 1H), 4.81 (s, 2H), 4.28 (q, J=7.4 Hz, 2H), 1.31 (t, J=7.4 Hz, 3H).

### 8-Bromo-3-oxo-3,4-dihydro-2H-benzo[b][1,4]oxazine-6-carboxylic acid

A stirred solution of ethyl 8-bromo-3-oxo-3,4-dihydro-2H-benzo[b][1,4]oxazine-6-carboxylate (10.0 g, 33.32 mmol) and NaOH (4.00 g, 99.96 mmol) in MeOH (167 mL) and water (50 mL) was heated at 60° C. for 48 hr. The reaction mixture was cooled below room temperature, acidified with conc. HCl, and the precipitate was filtered and dried to give the title compound (8.60 g, 95%).

### 8-Bromo-3-oxo-3,4-dihydro-2H-benzo[b][1,4]oxazine-6-carbonyl chloride

To a stirred solution of 8-bromo-3-oxo-3,4-dihydro-2H-benzo[b][1,4]oxazine-6-carboxylic acid (8.60 g, 31.61 mmol) in THF (158 mL) at room temperature was added oxalyl chloride (4.14 mL, 47.42 mmol), followed by several drops of DMF and stirring was continued for 6 hr. The reaction mixture was concentrated in vacuo and dried under high vacuum to give the title compound (9.00 g, 98%).

LCMS (ESI⁻) M−H⁻: 288, 290.

### 8-Bromo-N-methoxy-N-methyl-3-oxo-3,4-dihydro-2H-benzo[b][1,4]oxazine-6-carboxamide

To a stirred solution of 8-bromo-3-oxo-3,4-dihydro-2H-benzo[b][1,4]oxazine-6-carbonyl chloride (9.00 g, 30.98 mmol) and N-methoxymethanamine hydrochloride (6.04 g, 61.96 mmol) in DCM was added triethylamine (12.95 mL, 92.94 mmol) and the resulting mixture was stirred overnight. The reaction mixture was diluted with water, extracted three times with EtOAc, and the organic layer was dried (MgSO₄) and concentrated in vacuo to give the title compound as a yellow solid (4.20 g, 43%).

LCMS (ESI⁻) M−H⁻: 313, 315.

### 6-Acetyl-8-bromo-2H-benzo[b][1,4]oxazin-3(4H)-one

To a stirred solution of 8-bromo-N-methoxy-N-methyl-3-oxo-3,4-dihydro-2H-benzo[b][1,4]oxazine-6-carboxamide (2.50 g, 7.93 mmol) in THF at −78° C. was added dropwise MeMgCl (2.91 mL, 3.0 M in THF, 8.73 mmol), and the reaction mixture gradually warmed to room temperature. Water was added, the mixture was extracted three times with EtOAc, and the organic layer was dried (MgSO₄) and concentrated in vacuo. The residue was triturated with ether/petroleum ether to give the title compound was to give as a white solid (2.10 g, 98%).

LCMS (ESI⁻) M−H⁻: 268, 270.

### 8-Bromo-6-(4,4,4-trifluoro-3-oxobutanoyl)-2H-benzo[b][1,4]oxazin-3(4H)-one

According to the method in Example 71, ethyl 2,2,2-trifluoroacetate (4.42 mL, 37.03 mmol) and 6-acetyl-8-bromo-2H-benzo[b][1,4]oxazin-3(4H)-one (2.50 g, 9.26 mmol) were reacted to give the title compound as a yellow solid (660 mg, 19%).

LCMS (ESI⁻) M−H⁻: 364, 366.

### 8-Bromo-6-(1-(4-fluoro-2-methylphenyl)-3-(trifluoromethyl)-1H-pyrazol-5-yl)-2H-benzo[b][1,4]oxazin-3(4H)-one

According to the method in Example 71, 1-(4-fluoro-2-methylphenyl)hydrazine hydrochloride (318 mg, 1.80 mmol) and 8-bromo-6-(4,4,4-trifluoro-3-oxobutanoyl)-2H-benzo[b][1,4]oxazin-3(4H)-one (660 mg, 1.80 mmol) were reacted to give the title compound (275 mg, 32%).

¹H-NMR (400 MHz, DMSO-d₆) δ: 10.94 (s, 1H), 7.47 (dd, J=8.6, 5.5 Hz, 1H), 7.31 (dd, J=9.8, 3.1 Hz, 1H), 7.25 (s, 1H), 7.21 (td, J=8.6, 3.1 Hz, 1H), 7.19 (d, J=2.0 Hz, 1H), 6.63 (d, J=2.3 Hz, 1H), 4.71 (s, 2H), 1.91 (s, 3H); LCMS (ESI⁻) M−H⁻: 468, 470.

### Example 250

### 6-(1-(4-Fluoro-2-methylphenyl)-3-(trifluoromethyl)-1H-pyrazol-5-yl)indolin-2-one

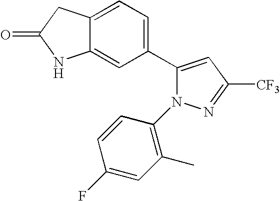

### 1-(4-Fluoro-2-methylphenyl)-3-(trifluoromethyl)-1H-pyrazol-5-ol

A stirred mixture of 1-(4-fluoro-2-methylphenyl)hydrazine hydrochloride (10.0 g, 56.62 mmol) and ethyl 4,4,4-trifluoro-3-oxobutanoate (10.0 g, 54.32 mmol) in IPA (20 mL) was reacted at 100° C. overnight. After cooling, petroleum ether was added with stirring, and the mixture was filtered to give the title compound as a white solid (3.0 g). Additional material was recovered by aqueous workup of the filtrate and trituration of the residue with petroleum ether (10.0 g).

LCMS (APCI⁻) M−H⁻: 259.

### 5-Bromo-1-(4-fluoro-2-methylphenyl)-3-(trifluoromethyl)-1H-pyrazole

A stirred mixture of 1-(4-fluoro-2-methylphenyl)-3-(trifluoromethyl)-1H-pyrazol-5-ol (6.8 g, 26 mmol) and phosphorous oxybromide (45.0 g, 157 mmol) was heated in a sealed tube at 155° C. for 30 hr. The mixture was poured cautiously into saturated NaHCO₃ and stirred until gas evolution stopped. The mixture was extracted with twice DCM, and the organic layer was washed with saturated NaHCO₃ and brine, dried (Mg₂SO₄), and concentrated in vacuo. The residue was passed through a plug of silica gel (DCM) to give the title compound as a yellow oil (7.0 g, 83%).

LCMS (ESI⁺): M+H⁺: 322, 324.

### 1-(4-Fluoro-2-methylphenyl)-3-(trifluoromethyl)-1H-pyrazol-5-ylboronic acid

To a stirred solution of 5-bromo-1-(4-fluoro-2-methylphenyl)-3-(trifluoromethyl)-1H-pyrazole (7.0 g, 21.7 mmol) in THF at −78° C. was slowly added n-butyllithium (13.5 mL, 1.6 M in hexanes, 21.7 mmol) and stirring was continued for 10 min before triisopropyl borate (6.48 mL, 28.2 mmol) was added. After stirring 30 min at −78° C., the reaction mixture warmed to −15° C., quenched with saturated NH₄Cl, and stirred at room temperature for 1 hr. The mixture was extracted with EtOAc, and the organic layer was washed with brine, dried (Na₂SO₄) and concentrated in vacuo to give the title compound as a cream solid (6.3 g, 100%).

LCMS (ESI⁻) M+H⁺: 289.

### 6-(1-(4-Fluoro-2-methylphenyl)-3-(trifluoromethyl)-1H-pyrazol-5-yl)indolin-2-one

A mixture of 1-(4-fluoro-2-methylphenyl)-3-(trifluoromethyl)-1H-pyrazol-5-ylboronic acid (109 mg, 0.34 mmol), 6-bromoindolin-2-one (24 mg, 0.11 mmol), potassium acetate (33 mg, 0.34 mmol) and dppf (3.8 mg, 0.0068 mmol) in degassed dioxane (3 mL) was evacuated and purged three times with N₂ on a vacuum manifold. PdCl₂(dppf)-DCM (11 mg, 0.014 mmol) was added and the mixture was again evacuated and purged. The reaction mixture was heated to 90° C. for 12 hr, poured into water, and extracted twice with EtOAc. The organic layer was washed with brine, dried (Na₂SO₄) and concentrated in vacuo. Flash chromatography of the residue on silica gel (5/1 hexane:EtOAc) gave the title compound as an off-white solid (25 mg, 59%).

¹H-NMR (400 MHz, CDCl₃) δ: 8.18 (brs, 1H), 7.26 (m, 1H), 7.13 (d, J=8.1 Hz, 1H), 6.97 (m, 2H), 6.78 (dd, J=7.6, 1.5 Hz, 1H), 6.78 (s, 1H), 6.69 (d, J=1.5 Hz, 1H), 3.51 (s, 2H), 1.98 (s, 3H); LCMS (APCI⁻) M−H⁻: 374.

### Example 251

### 6-(1-(4-Fluoro-2-methylphenyl)-3-(trifluoromethyl)-1H-pyrazol-5-yl)-2H-pyrido[3,2-b][1,4]oxazin-3(4H)-one

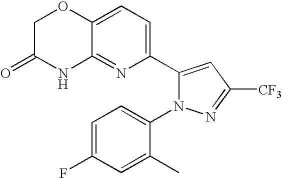

According to the method of Example 250, 1-(4-fluoro-2-methylphenyl)-3-(trifluoromethyl)-1H-pyrazol-5-ylboronic acid (330 mg, 1.15 mmol) and 6-bromo-2H-pyrido[3,2-b][1,4]oxazin-3(4H)-one (105 mg, 0.46 mmol) were reacted to give, after RP-HPLC purification, the title compound as a white solid (64 mg, 35%).

¹H-NMR (400 MHz, CDCl₃) δ: 7.69 (brs, 1H), 7.24 (m, 1H), 7.16 (d, J=8.2 Hz, 1H), 6.98 (m, 3H), 6.84 (d, J=8.2 Hz, 1H), 4.67 (s, 2H), 1.99 (s, 3H); LCMS (ESI⁻) M+H⁺: 393.

### Example 252

### 7-(1-(4-Fluoro-2-methylphenyl)-3-(trifluoromethyl)-1H-pyrazol-5-yl)-1H-pyrido[2,3-b][1,4]oxazin-2(3H)-one

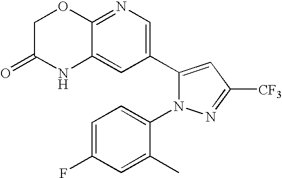

According to the method of Example 250, 1-(4-fluoro-2-methylphenyl)-3-(trifluoromethyl)-1H-pyrazol-5-ylboronic acid (189 mg, 0.65 mmol) and 7-bromo-1H-pyrido[2,3-b][1,4]oxazin-2(3H)-one (60 mg, 0.26 mmol) were reacted to give, after RP-HPLC purification, the title compound as a dark yellow solid (6.0 mg, 6%).

¹H-NMR (400 MHz, CDCl₃) δ: 8.17 (brs, 1H), 7.78 (d, J=2.0 Hz, 1H), 7.25 (m, 1H), 7.00 (m, 2H), 6.82 (s, 1H), 6.81 (d, J=2.0 Hz, 1H), 4.85 (s, 2H), 1.99 (s, 3H); LCMS (APCI⁺) M+H⁺: 393.

### Example 253

### 7-(1-(4-Fluoro-2-methylphenyl)-3-(trifluoromethyl)-1H-pyrazol-5-yl)-1H-pyrido[3,4-b][1,4]oxazin-2(3H)-one

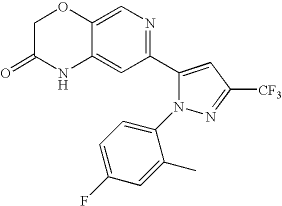

According to the method of Example 250, 7-bromo-1H-pyrido[3,4-b][1,4]oxazin-2(3H)-one (55.0 mg, 0.240 mmol, WO2006/010040) and 1-(4-fluoro-2-methylphenyl)-3-(trifluoromethyl)-1H-pyrazol-5-ylboronic acid (69.2 mg, 0.240 mmol) were reacted to give the title compound as a white solid (24.0 mg, 25%).

¹H-NMR (400 MHz, CDCl₃) δ: 8.16 (s, 1H), 7.81 (brs, 1H), 7.26 (s, 1H), 7.05 (s, 1H), 6.99 (m, 2H), 6.51 (s, 1H), 4.70 (s, 2H), 2.00 (s, 3H); LCMS (ESI⁺) M+H⁺: 393.

### Example 254

### 2-(1-(4-Fluoro-2-methylphenyl)-3-(trifluoromethyl)-1H-pyrazol-5-yl)-6H-pyrimido[5,4-b][1,4]oxazin-7(8H)-one

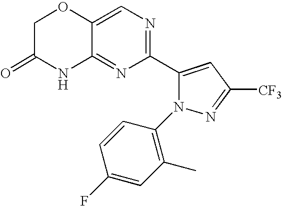

### 2-Chloro-5-methoxypyrimidin-4-amine

2,4-Dichloro-5-methoxypyrimidine (9.8 g, 55 mmol) in dioxane (20 mL) and ammonia (20 mL, 55 mmol) were stirred at 100° C. in a sealed tube overnight and then cooled to room temperature. The solids were filtered, washed with water and dried in vacuo to give the title compound as white crystals. The filtrate was partitioned between water and EtOAc, and the EtOAc layer was dried (Na₂SO₄) and concentrated in vacuo. Trituration of the residue (ether/petroleum ether), gave a second batch. Total yield of white solid was 8.6 g (98%).

¹H-NMR (400 MHz, DMSO-d₆) δ: 7.63 (s, 1H), 7.30 (brs, 2H), 3.76 (s, 3H).

### 4-Amino-2-chloropyrimidin-5-ol

To a stirred solution of 2-chloro-5-methoxypyrimidin-4-amine (1.0 g, 6.27 mmol) in DCM (200 mL) was added boron tribromide (8.9 mL, 94.0 mmol) with rapid stirring, and stirring was continued overnight. MeOH was added cautiously until the solution was homogenous, and the mixture was concentrated in vacuo. Water was added to the residue, the mixture was extracted with EtOAc, and the organic layer was dried (Na₂SO₄), and concentrated in vacuo. Trituration of the residue gave the title compound as a white solid (150 mg).

To the aqueous layer was added NaCl, the mixture was extracted three times with EtOAc containing 5% THF, and the organic layer was dried (Na₂SO₄), and concentrated to give additional material (300 mg). The aqueous layer was then again extracted with 3/1 DCM:IPA, and the organic layer was dried (Na₂SO₄), and concentrated in vacuo to provide further material (100 mg). Total yield of the title compound was 550 mg (60%) as white solid.

LCMS (APCI⁺) M+H⁺: 146.

### 2-Chloro-6H-pyrimido[5,4-b][1,4]oxazin-7(8H)-one

To a stirred solution of 4-amino-2-chloropyrimidin-5-ol (73 mg, 0.50 mmol) in THF (5 mL) and 2N Na₂CO₃ (5 mL) at room temperature was added chloroacetyl chloride (40 μL, 0.50 mmol) and stirring was continued overnight. The mixture was brought to reflux for 1 hr and then cooled, extracted with EtOAc, and the organic layer was dried (MgSO₄), and concentrated in vacuo. Flash chromatography of the residue on silica gel (4/1 hexane:EtOAc) gave the title compound as white solid (14 mg, 15%).

LCMS (APCI⁻) M−H⁻: 184.

### 2-(1-(4-Fluoro-2-methylphenyl)-3-(trifluoromethyl)-1H-pyrazol-5-yl)-6H-pyrimido[5,4-b][1,4]oxazin-7(8H)-one

According to the method of Example 250, 1-(4-fluoro-2-methylphenyl)-3-(trifluoromethyl)-1H-pyrazol-5-ylboronic acid (30 mg, 0.10 mmol) and 2-chloro-6H-pyrimido[5,4-b][1,4]oxazin-7(8H)-one (10 mg, 0.054 mmol) were reacted to give, after preparative TLC on silica gel (3:2 hexanes/EtOAc), the title compound as a white solid (5 mg, 24%).

¹H-NMR (400 MHz, CDCl₃) δ: 8.16 (s, 1H), 7.78 (brs, 1H), 7.32 (s, 1H), 7.20 (dd, J=8.6, 5.1 Hz, 1H), 7.00 (dd, J=9.0, 2.7 Hz, 1H), 6.95 (m, 1H), 4.74 (s, 2H), 1.99 (s, 3H);

LCMS (APCI⁻) M−H⁻: 392.

### Example 255

### 6-(1-(4-Fluoro-2-methylphenyl)-3-(trifluoromethyl)-1H-pyrazol-5-yl)-8-methyl-2H-pyrido[3,2-b][1,4]oxazin-3(4H)-one

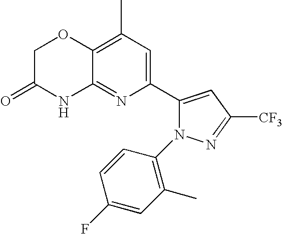

### 1-(4-Fluoro-2-methylphenyl)-3-(trifluoromethyl)-1H-pyrazol-5-ol

A stirred mixture of 1-(4-fluoro-2-methylphenyl)hydrazine hydrochloride (10.0 g, 56.62 mmol) and ethyl 4,4,4-trifluoro-3-oxobutanoate (10.0 g, 54.32 mmol) in IPA (20 mL) was reacted at 100° C. overnight. After cooling, petroleum ether was added with stirring, and the mixture was filtered to give the title compound as a white solid (3.0 g). Additional material was recovered by aqueous workup of the filtrate and trituration of the residue with petroleum ether (10.0 g).

¹H-NMR (400 MHz, DMSO-d₆) δ: 12.02 (brs, 1H), 7.33 (m, 1H), 7.24 (m, 1H), 7.12 (m, 1H), 5.85 (s, 1H), 2.01 (s, 3H), LCMS (APCI⁻) M−H⁻: 259.

### 5-Bromo-1-(4-fluoro-2-methylphenyl)-3-(trifluoromethyl)-1H-pyrazole

A stirred mixture of 1-(4-fluoro-2-methylphenyl)-3-(trifluoromethyl)-1H-pyrazol-5-ol (6.8 g, 26 mmol) and phosphorous oxybromide (45.0 g, 157 mmol) was heated in a sealed tube at 155° C. for 30 hr. The mixture was poured cautiously into saturated NaHCO₃ and stirred until gas evolution stopped. The mixture was extracted twice with DCM, and the organic layer was washed with saturated NaHCO₃ and brine, dried (Mg₂SO₄), and concentrated in vacuo. The residue was passed through a plug of silica gel (DCM) to give the title compound as a yellow oil (7.0 g, 83%).

¹H-NMR (400 MHz, CDCl₃) δ: 7.27 (dd, J=8.6, 5.1 Hz, 1H), 7.00-7.08 (m, 2H), 6.75 (s, 1H), 2.07 (s, 3H).

### 1-(4-Fluoro-2-methylphenyl)-3-(trifluoromethyl)-1H-pyrazol-5-ylboronic acid

To a stirred solution of 5-bromo-1-(4-fluoro-2-methylphenyl)-3-(trifluoromethyl)-1H-pyrazole (7.0 g, 21.7 mmol) in THF at −78° C. was slowly added n-butyllithium (13.5 mL, 1.6 M in hexanes, 21.7 mmol) and stirring was continued for 10 min before triisopropyl borate (6.48 mL, 28.2 mmol) was added. After stirring 30 min at −78° C., the reaction mixture was warmed to −15° C., quenched with saturated NH₄Cl, and stirred at room temperature for 1 hr. The mixture was extracted with EtOAc, and the organic layer was washed with brine, dried (Na₂SO₄) and concentrated in vacuo to give the title compound as a cream solid (6.3 g, 100%).

LCMS (ESI⁻) M+H⁺: 289.

### 6-(1-(4-Fluoro-2-methylphenyl)-3-(trifluoromethyl)-1H-pyrazol-5-yl)-8-methyl-2H-pyrido[3,2-b][1,4]oxazin-3(4H)-one

A mixture of 1-(4-fluoro-2-methylphenyl)-3-(trifluoromethyl)-1H-pyrazol-5-ylboronic acid (65 mg, 0.23 mmol), 6-bromo-8-methyl-2H-pyrido[3,2-b][1,4]oxazin-3(4H)-one (50 mg, 0.21 mmol), KOAc (61 mg, 0.62 mmol) and dppf (6.92 mg, 0.0123 mmol) in degassed dioxane (2 mL) was evacuated and purged (N₂) 3× on vacuum manifold. PdCl₂(dppf)-DCM (20.3 mg, 0.025 mmol) was added, and the mixture was again evacuated and purged, before heating at 90° C. overnight. The reaction mixture was poured into water, extracted twice with EtOAc, and the organic layer was washed with brine, dried (Na₂SO₄) and concentrated in vacuo. Flash chromatography of the residue on silica gel (10/1-7/1 hexane:EtOAc) gave the title compound as white solid (70 mg, 84%).

¹H-NMR (400 MHz, CDCl₃) δ: 7.78 (brs, 1H), 7.23 (dd, J=8.8, 5.2 Hz, 1H), 6.97 (m, 2H), 6.96 (s, 1H), 6.78 (s, 1H), 4.67 (s, 2H), 2.18 (s, 3H), 1.99 (s, 3H); LCMS (APCI⁻) M−H⁻: 405.

### Example 256

### 6-(3-Amino-1-(4-fluorophenyl)-1H-pyrazol-5-yl)-2H-benzo[b][1,4]oxazin-3(4H)-one

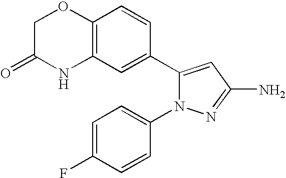

### 1-(4-Fluorophenyl)-5-(3-oxo-3,4-dihydro-2H-benzo[b][1,4]oxazin-6-yl)-1H-pyrazole-3-carboxylic acid

A mixture of ethyl 1-(4-fluorophenyl)-5-(3-oxo-3,4-dihydro-2H-benzo[b][1,4]oxazin-6-yl)-1H-pyrazole-3-carboxylate (1.00 g, 2.62 mmol, Example 102) in THF and 1N NaOH (6.56 mL, 6.56 mmol) was refluxed overnight. The mixture was cooled in an ice bath, 1N HCl was added to adjust to pH˜4, and the precipitated white solid was filtered, washed with water, and dried to give the title compound (0.78 g, 85%).

¹H-NMR (400 MHz, DMSO-d₆) δ: 12.99 (brs, 1H), 10.75 (brs, 1H), 7.39 (m, 2H), 7.32 (m, 2H), 6.96 (s, 1H), 6.93 (d, J=8.2 Hz, 1H), 6.79 (dd, J=8.2, 2.1 Hz, 1H), 6.77 (d, J=2.1 Hz, 1H), 4.60 (s, 2H); LCMS (ESI⁺) M+H⁺: 354.

### Benzyl 1-(4-fluorophenyl)-5-(3-oxo-3,4-dihydro-2H-benzo[b][1,4]oxazin-6-yl)-1H-pyrazol-3-yl carbamate

To a stirred solution of 1-(4-fluorophenyl)-5-(3-oxo-3,4-dihydro-2H-benzo[b][1,4]oxazin-6-yl)-1H-pyrazole-3-carboxylic acid (100 mg, 0.28 mmol) and diphenyl phosphoryl azide (134 μL, 0.62 mmol) in THF (5 mL) and DMF (2 mL) at 0° C. was added triethylamine (91 μL, 0.65 mmol) dropwise. The bath was removed and stirring was continued for 24 hr at room temperature. Benzyl alcohol (0.29 mL, 2.83 mmol) and toluene (10 mL) were added, the reaction temperature was increased to 100° C. with removal of THF, and heating was continued overnight. The reaction mixture was diluted with EtOAc, washed with water, 1N NaOH and brine, dried over Na₂SO₄ and concentrated in vacuo. Most of the material was taken forward as crude; however a small portion was purified by preparative TLC (5% MeOH in DCM) to give the title compound as a white film.

¹H-NMR (400 MHz, CDCl₃) δ: 9.30 (brs, 1H), 9.01 (brs, 1H), 7.30-7.40 (m, 5H), 7.17 (m, 2H), 6.96 (m, 2H), 6.84 (d, J=8.6 Hz, 1H), 6.70 (dd, J=8.6, 1.6 Hz, 1H), 6.53 (d, J=1.6 Hz, 1H), 5.22 (s, 2H), 4.62 (s, 2H); LCMS (ESI⁺) M+H⁺: 459.

### 6-(3-Amino-1-(4-fluorophenyl)-1H-pyrazol-5-yl)-2H-benzo[b][1,4]oxazin-3(4H)-one

Benzyl 1-(4-fluorophenyl)-5-(3-oxo-3,4-dihydro-2H-benzo[b][1,4]oxazin-6-yl)-1H-pyrazol-3-ylcarbamate in 1:1 MeOH/EtOAc was stirred overnight under 1 atm. of H₂ in the presence of 10% Pd/C. The mixture was filtered through Celite and the filtrate was concentrated in vacuo. Most of the material was taken forward as crude; however a small portion was purified by preparative TLC (10% MeOH in DCM) to give the title compound as an off-white powder.

¹H-NMR (400 MHz, CDCl₃+MeOD) δ: 7.20 (m, 2H), 7.01 (t, J=8.6 Hz, 2H), 6.88 (d, J=8.6 Hz, 1H), 6.78 (dd, J=8.6, 2.0 Hz, 1H), 6.65 (d, j=2.0 Hz, 1H), 5.86 (s, 1H), 4.60 (s, 2H); LCMS (ESI⁺) M+H⁺: 325.

### Example 257

### 6-(1-(4-Fluorophenyl)-3-(hydroxymethyl)-1H-pyrazol-5-yl)-2H-benzo[b][1,4]oxazin-3(4H)-one

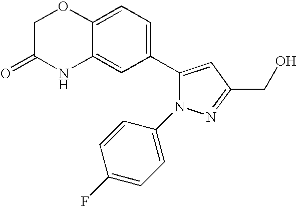

To a stirred solution of ethyl 1-(4-fluorophenyl)-5-(3-oxo-3,4-dihydro-2H-benzo[b][1,4]oxazin-6-yl)-1H-pyrazole-3-carboxylate (2 g, 5.2 mmol, Example 102) in THF at 0° C. was added lithium aluminum hydride (10.0 mL, 1.0 M in THF, 10.0 mmol) and the mixture stirred 30 min at 0° C., and then 4 hr at room temperature. The mixture was diluted with THF and quenched with sodium sulfate decahydrate. Water was added followed by 1N NaOH, and the mixture stirred at room temperature for 30 min. The mixture was poured into water, extracted with EtOAc, and the organic layer was washed with brine, dried (MgSO₄) and concentrated in vacuo to give the title compound as a tan solid (1.7 g, 96%).

¹H-NMR (400 MHz, MeOD) δ: 7.29 (m, 2H), 7.14 (m, 2H), 6.89 (d, J=8.4 Hz, 1H), 6.82 (dd, J=8.4, 2.0 Hz, 1H), 6.76 (d, J=2.0 Hz, 1H), 6.55 (s, 1H), 4.65 (s, 2H), 4.57 (s, 2H); LCMS (ESI⁺) M+H⁺: 340.

### Example 258

### 6-(3-(Fluoromethyl)-1-(4-fluorophenyl)-1H-pyrazol-5-yl)-2H-benzo[b][1,4]oxazin-3(4H)-one

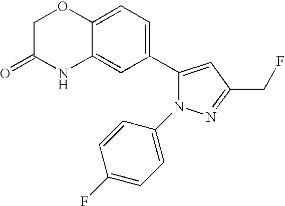

To a stirred solution of 6-(1-(4-fluorophenyl)-3-(hydroxymethyl)-1H-pyrazol-5-yl)-2H-benzo[b][1,4]oxazin-3(4H)-one (14 mg, 0.04 mmol) in DCM (1 mL) was added (diethylamino)sulfur trifluoride (15 μL, 0.11 mmol) at 0° C. The bath was removed and the mixture was stirred for 20 min at room temperature before pouring cautiously into saturated NaHCO₃. The mixture was extracted with EtOAc, and the organic layer was washed with brine, dried (Na₂SO₄) and concentrated in vacuo. Purification by preparative TLC (4% MeOH in DCM) gave the title compound as a white solid, (8 mg, 57%).

¹H-NMR (400 MHz, CDCl₃) δ: 8.41 (brs, 1H), 7.28 (m, 2H), 7.06 (m, 2H), 6.91 (d, J=8.4 Hz, 1H), 6.80 (dd, J=8.4, 2.0 Hz, 1H), 6.67 (d, J=2.0 Hz, 1H), 6.58 (d, J=1.5 Hz, 1H), 5.46 (d, J=48.4 Hz, 2H), 4.64 (s, 2H); LCMS (ESI⁺) M+H⁺: 342.

### Example 259

### (E)-Methyl 3-(1-(4-fluorophenyl)-5-(3-oxo-3,4-dihydro-2H-benzo[b][1,4]oxazin-6-yl)-1H-pyrazol-3-yl)acrylate

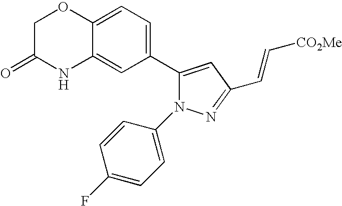

### 1-(4-Fluorophenyl)-5-(3-oxo-3,4-dihydro-2H-benzo[b][1,4]oxazin-6-yl)-1H-pyrazole-3-carbaldehyde

To a stirred suspension of ethyl 1-(4-fluorophenyl)-5-(3-oxo-3,4-dihydro-2H-benzo[b][1,4]oxazin-6-yl)-1H-pyrazole-3-carboxylate (2 g, 5.2 mmol, Example 102) in dry DCM (120 mL) at −78° C. was added diisobutylaluminum hydride (7.0 mL, 1.5 M in THF, 10 mmol) and stirring was continued for 2 hr. The mixture was quenched by the slow addition of MeOH and allowed to come to room temperature. The mixture was diluted with EtOAc, washed with saturated NH₄Cl solution, brine, saturated NaHCO₃, brine, dried (MgSO₄), and concentrated in vacuo. Flash chromatography of the residue on silica gel (0-20% EtOAc in DCM) gave the title compound as a yellow foam (1.2 g, 68%) containing the starting ester as impurity.

¹H-NMR (400 MHz, CDCl₃) δ: 10.05 (s, 1H), 8.73 (brs, 1H), 7.34 (m, 2H), 7.11 (m, 2H), 6.97 (s, 1H), 6.93 (d, J=8.2 Hz, 1H), 6.78 (dd, J=8.2, 2.0 Hz, 1H), 6.70 (d, J=2.0 Hz, 1H), 4.65 (s, 2H); LCMS (ESI⁻), M−H⁻: 336.

### (E)-Methyl 3-(1-(4-fluorophenyl)-5-(3-oxo-3,4-dihydro-2H-benzo[b][1,4]oxazin-6-yl)-1H-pyrazol-3-yl)acrylate

To a stirred solution of trimethyl phosphonoacetate (0.29 mL, 1.78 mmol) in THF at −78° C. was added n-butyllithium (0.71 mL, 2.5 M in hexanes, 1.78 mmol) dropwise and the mixture was stirred 40 min at this temperature. A solution of 1-(4-fluorophenyl)-5-(3-oxo-3,4-dihydro-2H-benzo[b][1,4]oxazin-6-yl)-1H-pyrazole-3-carbaldehyde (240 mg, 0.72 mmol) in THF was added dropwise. After 20 min, the bath was removed and the mixture was warmed to room temperature for 5 hr. The reaction mixture was quenched with saturated NH₄Cl solution, extracted with EtOAc, and the organic layer was washed with brine, dried (MgSO₄) and concentrated in vacuo. Flash chromatography of the residue on silica gel (0-20% EtOAc in DCM) gave the title compound as a clear film (210 mg, 75%).

¹H-NMR (400 MHz, CDCl₃) δ: 8.95 (brs, 1H), 7.72 (d, J=15.8 Hz, 1H), 7.29 (m, 2H), 7.06 (m, 2H), 6.91 (d, J=8.2 Hz, 1H), 6.79 (d, J=8.4 Hz, 1H), 6.70 (s, 1H), 6.67 (s, 1H), 6.51 (d, J=15.8 Hz, 1H), 4.64 (s, 2H), 3.82 (s, 3H); LCMS (ESI⁺) M+H⁺: 394.

### Example 260

### Methyl 3-(1-(4-fluorophenyl)-5-(3-oxo-3,4-dihydro-2H-benzo[b][1,4]oxazin-6-yl)-1H-pyrazol-3-yl)propanoate

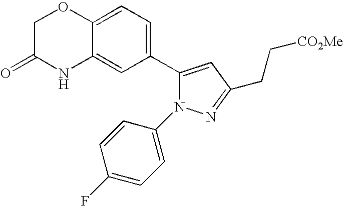

(E)-Methyl 3-(1-(4-fluorophenyl)-5-(3-oxo-3,4-dihydro-2H-benzo[b][1,4]oxazin-6-yl)-1H-pyrazol-3-yl)acrylate (190 mg, 0.48 mmol) in MeOH was treated overnight under 1 atm. of H₂ in the presence of 10% Pd/C. The mixture was filtered through Celite and concentrated in vacuo. Most of the material was taken forward as crude; however a small portion was purified by preparative TLC (30% EtOAc in DCM) to give the title compound as white powder.

¹H-NMR (400 MHz, CDCl₃) δ: 9.39 (brs, 1H), 7.23 (m, 2H), 7.02 (m, 2H), 6.87 (d, J=8.4 Hz, 1H), 6.75 (dd, J=8.4, 2.0 Hz, 1H), 6.69 (d, J=2.0 Hz, 1H), 6.28 (s, 1H), 4.62 (s, 2H), 3.71 (s, 3H), 3.04 (t, J=7.6 Hz, 2H), 2.77 (t, J=7.6 Hz, 2H); LCMS (ESI⁺) M+H⁺: 396.

### Example 261

### 6-(1-(4-Fluorophenyl)-3-(3-hydroxypropyl)-1H-pyrazol-5-yl)-2H-benzo[b][1,4]oxazin-3(4H)-one

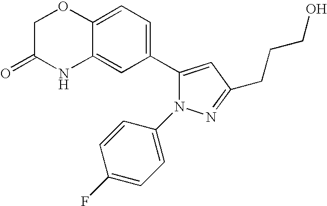

To a stirred solution of methyl 3-(1-(4-fluorophenyl)-5-(3-oxo-3,4-dihydro-2H-benzo[b][1,4]oxazin-6-yl)-1H-pyrazol-3-yl)propanoate (20 mg, 0.05 mmol) in THF (3 mL) at 0° C. was added dropwise lithium aluminum hydride (0.10 mL, 1.0 M in THF, 0.10 mmol), and the mixture was warmed to room temperature overnight. The mixture was quenched by dropwise addition of water, 2N NaOH, and water, and extracted with EtOAc, and the organic layer was washed with brine, dried (Na₂SO₄) and concentrated in vacuo. Purification of the residue by preparative TLC gave the title compound as a white foam (12 mg, 65%).

¹H-NMR (400 MHz, CDCl₃+acetone-d₆) δ: 9.28 (brs, 1H), 7.22 (m, 2H), 7.00 (m, 2H), 6.87 (d, J=8.4 Hz, 1H), 6.75 (dd, J=8.4, 2.0 Hz, 1H), 6.69 (d, J=2.0 Hz, 1H), 6.28 (s, 1H), 4.62 (s, 2H), 3.75 (t, J=6.2 Hz, 2H), 2.82 (t, J=7.2 Hz, 2H), 1.98 (m, 2H); LCMS (ESI⁺) M+H⁺: 368.

### Example 262

### 6-(1-(4-Fluorophenyl)-3-(1-hydroxyethyl)-1H-pyrazol-5-yl)-2H-benzo[b][1,4]oxazin-3(4H)-one

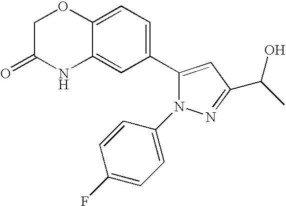

To a stirred solution of 1-(4-fluorophenyl)-5-(3-oxo-3,4-dihydro-2H-benzo[b][1,4]oxazin-6-yl)-1H-pyrazole-3-carbaldehyde (500 mg, 1.48 mmol) in THF (20 mL) at 0° C. was added dropwise methylmagnesium bromide (1.98 mL, 1.5 M in ether, 2.96 mmol). The reaction mixture was stirred 1 hr, and then warmed to room temperature for 4 hr. The mixture was quenched with saturated NH₄Cl solution, extracted with EtOAc, and the organic layer was washed with brine, dried (Na₂SO₄) and concentrated in vacuo. Flash chromatography of the residue on silica gel (0-10% MeOH in DCM) gave the title compound (410 mg, 78%) as a white solid.

¹H-NMR (400 MHz, CDCl₃+MeOD) δ: 7.26 (m, 2H), 7.06 (m, 2H), 6.86 (dd, J=7.8, 1.0 Hz, 1H), 6.74 (m, 2H), 6.47 (s, 1H), 4.98 (q, J=6.6 Hz, 1H), 4.60 (s, 2H), 1.59 (d, J=6.6 Hz, 3H); LCMS (ESI⁺) (M+H—H₂O)⁺: 336.

### Example 263

### 6-(1-(4-Fluorophenyl)-3-(2,2,2-trifluoro-1-hydroxyethyl)-1H-pyrazol-5-yl)-2H-benzo[b][1,4]oxazin-3(4H)-one

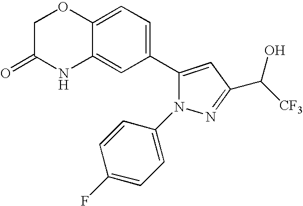

To a stirred solution of 1-(4-fluorophenyl)-5-(3-oxo-3,4-dihydro-2H-benzo[b][1,4]oxazin-6-yl)-1H-pyrazole-3-carbaldehyde (120 mg, 0.36 mmol) in THF at 0° C. was added trimethyl(trifluoromethyl)silane (1.42 mL, 0.5 M in THF, 0.71 mmol) followed by tetrabutylammonium fluoride (0.71 mL, 1.0 M in THF, 0.71 mmol) and the mixture was stirred 2 days at room temperature. After this time, additional trimethyl(trifluoromethyl)silane (1.42 mL) and tetrabutylammonium fluoride (0.71 mL) were added and the mixture was brought to reflux for 6 hr. The mixture was cooled, quenched with saturated NH₄Cl solution, extracted three times with EtOAc, and the organic layer was washed with brine, dried (MgSO₄) and concentrated in vacuo. Flash chromatography of the residue on silica gel (0-5% MeOH in DCM) gave the title compound as a pale yellow solid (71 mg, 49%).

¹H-NMR (400 MHz, CDCl₃) δ: 9.64 (brs, 1H), 7.94 (brs, 1H), 7.34 (s, 1H), 7.29 (m, 2H), 7.10 (m, 2H), 6.94 (d, J=8.4 Hz, 1H), 6.81 (dd, J=8.4, 2.0 Hz, 1H), 6.73 (s, 1H), 6.67 (d, J=2.0 Hz, 1H), 4.65 (s, 2H); LCMS (ESI⁻), M−H⁻: 406.

### Example 264

### 6-(3-Ethyl-1-(4-fluorophenyl)-1H-pyrazol-5-yl)-2H-benzo[b][1,4]oxazin-3(4H)-one

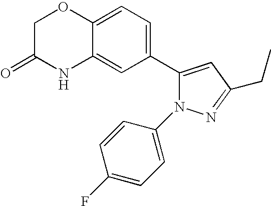

To a stirred solution of 6-(1-(4-fluorophenyl)-3-(1-hydroxyethyl)-1H-pyrazol-5-yl)-2H-benzo[b][1,4]oxazin-3(4H)-one (10 mg, 0.028 mmol) in DCM (2 mL) and triethylsilane (1 mL, 6.26 mmol) was added dropwise TFA (0.5 mL, 6.49 mmol) at room temperature. The mixture was stirred 3 days at room temperature and then at reflux overnight. Concentration in vacuo and preparative TLC of the residue gave the title compound as a white solid (8 mg, 84%).

¹H-NMR (400 MHz, CDCl₃) δ: 8.72 (brs, 1H), 7.26 (m, 2H), 7.03 (m, 2H), 6.98 (d, J=8.4 Hz, 1H), 6.79 (dd, J=8.4, 2.0 Hz, 1H), 6.67 (d, J=2.0 Hz, 1H), 6.29 (s, 1H), 4.63 (s, 2H), 2.74 (q, J=7.6 Hz, 2H), 1.32 (t, J=7.6 Hz, 3H); LCMS (ESI⁺) M+H⁺: 338.

### Example 265

### 6-(3-Acetyl-1-(4-fluorophenyl)-1H-pyrazol-5-yl)-2H-benzo[b][1,4]oxazin-3(4H)-one

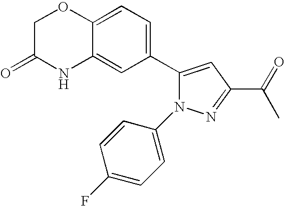

To a stirred suspension of 6-(1-(4-fluorophenyl)-3-(1-hydroxyethyl)-1H-pyrazol-5-yl)-2H-benzo[b][1,4]oxazin-3(4H)-one (50 mg, 0.14 mmol) in DCM (5 mL) was added manganese(IV) oxide (62 mg, 0.71 mmol) and the mixture refluxed overnight. After this time, additional manganese(IV) oxide (350 mg) and DCM (6 mL) were added and reflux was continued for 16 hr. The mixture was filtered through silica gel (EtOAc), and the residue purified by flash chromatography on silica gel (3/1 hexane:EtOAc) to give the title compound as a white solid (11 mg, 22%).

¹H-NMR (400 MHz, CDCl₃) δ: 8.76 (brs, 1H), 7.33 (m, 2H), 7.10 (m, 2H), 6.96 (s, 1H), 6.91 (d, J=8.4 Hz, 1H), 6.76 (dd, J=8.4, 1.6 Hz, 1H), 6.71 (d, J=1.6 Hz, 1H), 4.65 (s, 2H), 2.65 (s, 3H); LCMS (ESI⁺) M+H⁺: 352.

### Example 266

### 6-(3-(1-Fluoroethyl)-1-(4-fluorophenyl)-1H-pyrazol-5-yl)-2H-benzo[b][1,4]oxazin-3(4H)-one

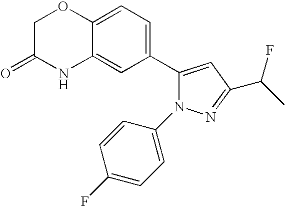

To a stirred solution of 6-(1-(4-fluorophenyl)-3-(1-hydroxyethyl)-1H-pyrazol-5-yl)-2H-benzo[b][1,4]oxazin-3(4H)-one (15 mg, 0.043 mmol) in DCM (5 mL) was added (diethylamino)sulfur trifluoride (56 μL, 0.43 mmol) at room temperature and stirring was continued for 5 min. Saturated NaHCO₃ solution was added cautiously, the mixture extracted with EtOAc, and the organic layer was washed with brine, dried (MgSO₄) and concentrated in vacuo. Purification of the residue by preparative TLC (4% MeOH in DCM) gave the title compound as a white solid (13 mg, 86%).

¹H-NMR (400 MHz, CDCl₃) δ: 9.12 (brs, 1H), 7.27 (m, 2H), 7.05 (m, 2H), 6.90 (d, J=8.2 Hz, 1H), 6.78 (dd, J=8.2, 2.0 Hz, 1H), 6.70 (d, J=2.0 Hz, 1H), 6.54 (s, 1H), 5.76 (dq, J=48.0, 6.4 Hz, 1H), 4.64 (s, 2H), 1.77 (dd, J=23.8, 6.4 Hz, 3H); LCMS (ESI⁺) (M+H)⁺: 356.

### Example 267

### 6-(3-(1,1-Difluoroethyl)-1-(4-fluorophenyl)-1H-pyrazol-5-yl)-2H-benzo[b][1,4]oxazin-3(4H)-one

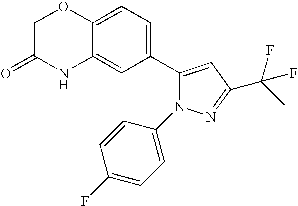

To Deoxo-Fluor(R) (1.0 mL, 5.46 mmol) at room temperature was added dropwise with stirring a solution of 6-(3-acetyl-1-(4-fluorophenyl)-1H-pyrazol-5-yl)-2H-benzo[b][1,4]oxazin-3(4H)-one (50 mg, 0.14 mmol) in DCM (1 mL). EtOH (1 drop) was added and stirring was continued for 30 hr. Purification of the residue by preparative TLC (2/1 hexane:EtOAc) gave the title compound as a tan solid (8 mg, 15%).

¹H-NMR (400 MHz, CDCl₃) δ: 8.11 (brs, 1H), 7.29 (m, 2H), 7.07 (m, 2H), 6.92 (d, J=8.4 Hz, 1H), 6.79 (dd, J=8.4, 1.6 Hz, 1H), 6.65 (d, J=1.6 Hz, 1H), 6.64 (s, 1H), 4.64 (s, 2H), 2.08 (t, J=18.5 Hz, 3H); LCMS (APCI⁻) (M−H)⁻: 372.

### Example 268

### 6-(1-(3-Hydroxyphenyl)-3-(trifluoromethyl)-1H-pyrazol-5-yl)-2H-benzo[b][1,4]oxazin-3(4H)-one

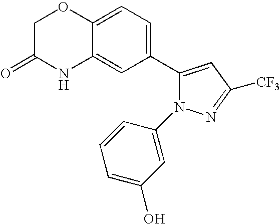

To a stirred solution of 6-(1-(3-methoxyphenyl)-3-(trifluoromethyl)-1H-pyrazol-5-yl)-2H-benzo[b][1,4]oxazin-3(4H)-one (50 mg, 0.13 mmol) in DCM (5 mL) was added boron tribromide (0.39 mL, 0.39 mmol) at 0° C. and the mixture was stirred overnight at room temperature. MeOH was added slowly until homogenous, the mixture was stirred 20 min, and then concentrated in vacuo. Flash chromatography of the residue on silica gel (2% MeOH in DCM) gave the title compound as a white solid (42 mg, 87%).

¹H-NMR (400 MHz, CDCl₃) δ: 8.68 (brs, 1H), 7.16 (m, 1H), 6.93 (d, J=8.2 Hz, 1H), 6.88 (dd, J=8.2, 1.6 Hz, 1H), 6.83 (m, 1H), 6.77 (m, 2H), 6.69 (s, 1H), 6.65 (d, J=1.6 Hz, 1H), 6.43 (brs, 1H), 4.63 (s, 2H); LCMS (ESI⁻) M−H⁻: 374.

### Example 269

### 6-(1-(4-Fluorophenyl)-1H-pyrazol-5-yl)-2H-benzo[b][1,4]oxazin-3(4H)-one

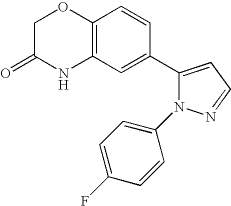

### 6-(3-(Dimethylamino)acryloyl)-2H-benzo[b][1,4]oxazin-3(4H)-one

A stirred suspension of 6-acetyl-2H-benzo[b][1,4]oxazin-3(4H)-one (4.0 g, 20.9 mmol) and N,N-dimethylformamide dimethyl acetal (4.46 mL, 33.5 mmol) in EtOH (50 mL) was heated to 80° C. 16 hr. The mixture was cooled, and the precipitated solid was filtered and washed with cold EtOH to give the title compound as a yellow solid (3.8 g, 73%).

LCMS (ESI⁺) M+H⁺: 247.

### 6-(1-(4-Fluorophenyl)-1H-pyrazol-5-yl)-2H-benzo[b][1,4]oxazin-3(4H)-one

To a stirred mixture of 1-(4-fluorophenyl)hydrazine hydrochloride (0.58 g, 3.54 mmol) in MeOH (14 mL) at 0° C. was added 6N HCl (3.16 mL, 19.0 mmol) and the mixture was stirred 5 min. 6-(3-(Dimethylamino)acryloyl)-2H-benzo[b][1,4]oxazin-3(4H)-one (0.78 g, 3.16 mmol) was added and the reaction mixture was brought to 40° C. for 16 hr. The reaction volume was reduced while cooling, and the precipitated solid was filtered, washed with petroleum ether, and recrystallized from EtOH to give the title compound as a tan solid (0.43 g, 44%).

¹H-NMR (400 MHz, CDCl₃) δ: 8.54 (brs, 1H), 7.69 (d, J=2.0 Hz, 1H), 7.28 (m, 2H), 7.05 (m, 2H), 6.91 (d, J=8.6 Hz, 1H) 6.80 (dd, 8.6. 2.0 Hz, 1H), 6.67 (d, J=2.0 Hz, 1H), 6.46 (d, J=2.0 Hz, 1H), 4.64 (s, 2H); LCMS (ESI⁺) M+H⁺: 310.

### Example 270

### 6-(4-Bromo-1-(4-fluorophenyl)-1H-pyrazol-5-yl)-2H-benzo[b][1,4]oxazin-3(4H)-one

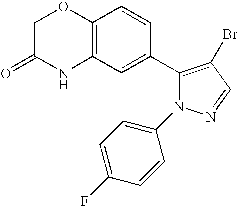

To a stirred solution of 6-(1-(4-fluorophenyl)-1H-pyrazol-5-yl)-2H-benzo[b][1,4]oxazin-3(4H)-one (90 mg, 0.29 mmol) in DMF (2 mL) at room temperature was added NBS (52 mg, 0.29 mmol) and stirring was continued for 2 hr. The mixture was diluted with water and cooled to 0° C., and filtered to give the title compound as a white solid (83 mg, 73%).

¹H-NMR (400 MHz, CDCl₃) δ: 8.24 (brs, 1H), 7.73 (s, 1H), 7.21 (m, 2H), 7.03 (m, 1H), 6.96 (d, J=8.6 Hz, 1H), 6.82 (dd, J=8.6, 2.0 Hz, 1H), 6.73 (d, J=2.0 Hz, 1H), 4.66 (s, 2H);

LCMS (ESI⁺) M+H⁺: 388, 390.

### Example 271

### 6-(4-Chloro-1-(4-fluorophenyl)-1H-pyrazol-5-yl)-2H-benzo[b][1,4]oxazin-3(4H)-one

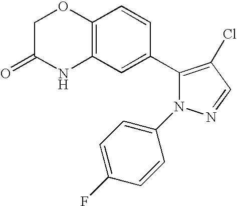

To a stirred solution of 6-(1-(4-fluorophenyl)-1H-pyrazol-5-yl)-2H-benzo[b][1,4]oxazin-3(4H)-one (100 mg, 0.32 mmol) in DMF (2 mL) at 53° C. was added a solution of NCS (43.2 mg, 0.32 mmol) in DMF (1 mL) and stirring was continued for 2 hr. The reaction mixture was cooled to room temperature, diluted with water, cooled again to 0° C. and filtered. Flash chromatography of the obtained solid on silica gel (10% EtOAc in DCM) gave the title compound as a white solid (87 mg, 78%).

¹H-NMR (400 MHz, CDCl₃) δ: 8.23 (brs, 1H), 7.70 (s, 1H), 7.22 (m, 2H), 7.04 (m, 2H), 6.96 (d, J=8.2 Hz, 1H), 6.82 (dd, J=8.2, 2.0 Hz, 1H), 6.74 (d, J=2.0 Hz, 1H), 4.66 (s, 2H); LCMS (ESI⁺) M+H⁺: 344.

### Example 272

### Ethyl 1-(4-fluorophenyl)-5-(3-oxo-3,4-dihydro-2H-benzo[b][1,4]oxazin-6-yl)-1H-pyrazole-4-carboxylate

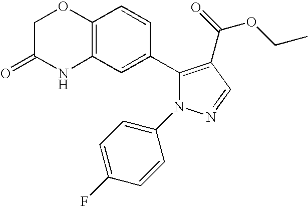

### Ethyl 3-oxo-3-(3-oxo-3,4-dihydro-2H-benzo[b][1,4]oxazin-6-yl)propanoate

According to the method of Example 71, diethyl carbonate (12.7 mL, 105 mmol) and 6-acetyl-2H-benzo[b][1,4]oxazin-3(4H)-one (10 g, 52.3 mmol) were reacted to give the title compound as a white solid (8.69 g, 63%).

LCMS (ESI⁻) M−H⁻: 262.

### Ethyl 3-(dimethylamino)-2-(3-oxo-3,4-dihydro-2H-benzo[b][1,4]oxazine-6-carbonyl)acrylate

According to the method of Example 269, ethyl 3-oxo-3-(3-oxo-3,4-dihydro-2H-benzo[b][1,4]oxazin-6-yl)propanoate (200 mg, 0.760 mmol) and N,N-dimethylformamide dimethyl acetal (162 μL, 1.22 mmol) were reacted in EtOH to give the title compound as an off white solid (230 mg, 95%).

LCMS (ESI⁻) M−H⁻: 317.

### Ethyl 1-(4-fluorophenyl)-5-(3-oxo-3,4-dihydro-2H-benzo[b][1,4]oxazin-6-yl)-1H-pyrazole-4-carboxylate

According to the method of Example 269, 1-(4-fluorophenyl)hydrazine hydrochloride (132 mg, 0.81 mmol) and ethyl 3-(dimethylamino)-2-(3-oxo-3,4-dihydro-2H-benzo[b][1,4]oxazine-6-carbonyl)acrylate (230 mg, 0.72 mmol) were reacted to give, after flash chromatography on silica (0-10% EtOAc in hexanes) the title compound as an off-white solid (37 mg, 13%).

¹H-NMR (400 MHz, CDCl₃) δ: 8.15 (brs, 1H), 7.95 (s, 1H), 7.20 (m, 2H), 7.02 (m, 2H), 6.92 (d, J=7.8 Hz, 1H), 6.81 (m, 2H), 4.65 (s, 2H), 4.24 (q, J=7.0 Hz, 2H), 1.29 (t, J=7.0 Hz, 3H); LCMS (ESI⁺) M+H⁺: 382.

### Example 273

### 6-(1-(4-Fluorophenyl)-4-iodo-1H-pyrazol-5-yl)-2H-benzo[b][1,4]oxazin-3(4H)-one

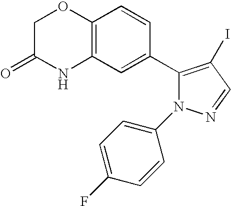

To a stirred solution of 6-(1-(4-fluorophenyl)-1H-pyrazol-5-yl)-2H-benzo[b][1,4]oxazin-3(4H)-one (1.0 g, 3.2 mmol) in DMF at 0° C. was added NIS (0.73 g, 3.2 mmol) and the reaction mixture was brought to 55° C. for 60 hr. The mixture was diluted with water, cooled to 0° C., and filtered. Trituration of the residue with DCM gave the title compound as a white solid (0.70 g, 50%).

¹H-NMR (400 MHz, CDCl₃) δ: 7.80 (brs, 1H), 7.76 (s, 1H), 7.19 (m, 2H), 7.02 (m, 2H), 6.97 (d, J=8.2 Hz, 1H), 6.83 (dd, J=8.2, 2.0 Hz, 1H), 6.69 (d, J=2.0 Hz, 1H), 4.67 (s, 2H);

LCMS (ESI⁺) M+H⁺: 436.

### Example 274

### 1-(4-Fluorophenyl)-5-(3-oxo-3,4-dihydro-2H-benzo[b][1,4]oxazin-6-yl)-1H-pyrazole-4-carbonitrile

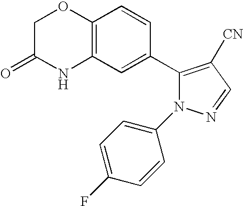

A stirred solution of 6-(4-bromo-1-(4-fluorophenyl)-1H-pyrazol-5-yl)-2H-benzo[b][1,4]oxazin-3(4H)-one (100 mg, 0.26 mmol) in DMA containing Zn(CN)₂ (30.2 mg, 0.26 mmol), zinc dust (4.0 mg, 0.06 mmol), dppf (11.4 mg, 0.021 mmol) and Pd₂ dba₃ (9.44 mg, 0.01 mmol) was degassed (N₂) and heated to 120° C. for 16 hr. The mixture was cooled, poured into water, extracted with EtOAc, and the organic layer was washed with brine, dried (MgSO₄), and concentrated in vacuo. Flash chromatography of the residue on silica (30% EtOAc in DCM) followed by preparative TLC (50% EtOAc in petroleum ether) gave the title compound as a white solid (26 mg, 30%).

¹H-NMR (400 MHz, CDCl₃) δ: 8.00 (brs, 1H), 7.87 (s, 1H), 7.28 (m, 2H), 7.10 (m, 2H), 6.98 (d, J=8.6 Hz, 1H), 6.85 (dd, J=8.6, 2.3 Hz, 1H), 6.80 (d, J=2.3 Hz, 1H), 4.68 (s, 2H),

LCMS (ESI⁺) M+H⁺: 335.

### Example 275

### 6-(1-(4-Fluorophenyl)-4-(trifluoromethyl)-1H-pyrazol-5-yl)-2H-benzo[b][1,4]oxazin-3(4H)-one

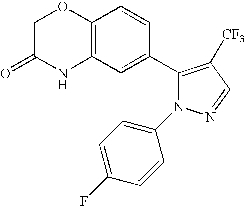

A mixture of 6-(1-(4-fluorophenyl)-4-iodo-1H-pyrazol-5-yl)-2H-benzo[b][1,4]oxazin-3(4H)-one (200 mg, 0.46 mmol), methyl 2,2-difluoro-2-(fluorosulfonyl)acetate (0.39 mL, 3.03 mmol) and cupper(I) iodide (96.3 mg, 0.51 mmol) in DMF (3 mL) was degassed (N₂) and heated at 100° C. for 16 hr. The reaction mixture was poured into water, extracted with EtOAc, and the organic layer was washed with water and brine, dried (MgSO₄), and concentrated in vacuo. Flash chromatography of the residue on silica (10% EtOAc in DCM) followed by preparative TLC (30% EtOAc in DCM) gave the title compound as a white solid (8.2 mg, 4.7%).

¹H-NMR (400 MHz, CDCl₃) δ: 7.93 (brs, 1H), 7.65 (s, 1H), 7.21 (m, 2H), 7.04 (m, 2H), 6.96 (d, J=8.2 Hz, 1H), 6.83 (dd, J=8.2, 2.0 Hz, 1H), 6.66 (d, J=2.0 Hz, 1H), 4.67 (s, 2H);

LCMS (ESI⁺) M+H⁺: 378.

### Example 276

### 6-(1-(4-Fluorophenyl)-4-methyl-1H-pyrazol-5-yl)-2H-benzo[b][1,4]oxazin-3(4H)-one

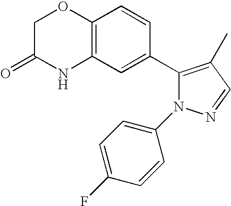

To a stirred solution of 6-(4-bromo-1-(4-fluorophenyl)-1H-pyrazol-5-yl)-2H-benzo[b][1,4]oxazin-3(4H)-one (100 mg, 0.26 mmol) in THF (2.5 mL) at −78° C. was added dropwise n-butyllithium (206 mL, 2.5 M in hexanes, 0.52 mmol) and stirring was continued 5 min at −78° C. Iodomethane (17 μL, 0.26 mmol) was added, and the mixture stirred an additional 10 min at −78° C. The reaction mixture was poured into water, extracted with EtOAc, and the organic layer was washed with brine, dried (MgSO₄), and concentrated in vacuo. Flash chromatography of the residue on silica gel (5% EtOAc in DCM) followed by RP-HPLC gave the title compound as a white solid (14 mg, 17%).

¹H-NMR (400 MHz, CDCl₃) δ: 7.73 (brs, 1H), 7.57 (s, 1H), 7.20 (m, 2H), 6.99 (m, 3H), 6.78 (dd, J=8.6, 2.0 Hz, 1H), 6.55 (d, J=2.0 Hz, 1H), 4.65 (s, 2H), 2.09 (s, 3H); LCMS (ESI⁺) M+H⁺: 324.

### Example 277

### 6-(4-Fluoro-1-(4-fluorophenyl)-1H-pyrazol-5-yl)-2H-benzo[b][1,4]oxazin-3(4H)-one

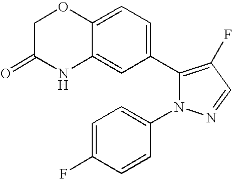

To a stirred solution of 6-(4-bromo-1-(4-fluorophenyl)-1H-pyrazol-5-yl)-2H-benzo[b][1,4]oxazin-3(4H)-one (100 mg, 0.26 mmol) in THF (2 mL) at −78° C. was added dropwise n-butyllithium (206 μL, 2.5 M in hexanes, 0.52 mmol) and stirring was continued for 15 min. at −78° C. The reaction mixture was transferred dropwise to a mixture of N-fluoro-N-(phenylsulfonyl)benzenesulfonamide (81.2 mg, 0.26 mmol) in THF (1 mL) at −78° C., the mixture was warmed slowly to room temperature, stirring was continued for 16 hr. The reaction mixture was poured into water, extracted with EtOAc, and the organic layer was washed with brine, dried (MgSO₄), and concentrated in vacuo. Flash chromatography of the residue on silica gel (10% EtOAc in DCM) gave the title compound as an off-white solid (10 mg, 11%).

¹H-NMR (400 MHz, CDCl₃) δ: 8.31 (brs, 1H), 7.62 (d, 1H), 7.06 (m, 2H), 6.94 (m, 2H), 6.73 (m, 3H), 4.65 (s, 2H); LCMS (ESI⁺) M+H⁺: 328.

### Example 278

### 6-(4-Chloro-1-(4-fluorophenyl)-3-(trifluoromethyl)-1H-pyrazol-5-yl)-2H-benzo[b][1,4]oxazin-3(4H)-on

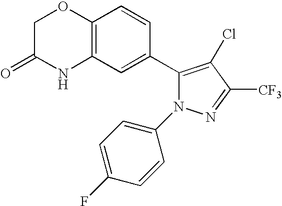

According to the method of Example 271, 6-(1-(4-fluorophenyl)-3-(trifluoromethyl)-1H-pyrazol-5-yl)-2H-benzo[b][1,4]oxazin-3(4H)-one (105 mg, 0.28 mmol) and NCS (74.3 mg, 0.557 mmol) were reacted to give the title compound as a white solid (30 mg, 26%).

¹H-NMR (400 MHz, CDCl₃) δ: 8.25 (brs, 1H), 7.25 (m, 2H), 7.07 (m, 2H), 6.98 (d, J=8.2 Hz, 1H), 6.82 (dd, J=8.2, 2.0 Hz, 1H), 6.74 (d, J=2.0 Hz, 1H), 4.68 (s, 2H); LCMS (ESI⁺) M+H⁺: 412.

### 7-Chloro-6-(1-(4-fluorophenyl)-3-(trifluoromethyl)-1H-pyrazol-5-yl)-2H-benzo[b][1,4]oxazin-3(4H)-one

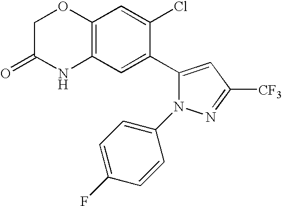

7-Chloro-6-(1-(4-fluorophenyl)-3-(trifluoromethyl)-1H-pyrazol-5-yl)-2H-benzo[b][1,4]oxazin-3(4H)-one was also isolated as a white solid (12 mg, 10%).

¹H-NMR (400 MHz, CDCl₃) δ: 7.74 (s, 1H), 7.28 (m, 2H), 7.05 (s, 1H), 7.04 (m, 2H), 6.72 (s, 1H) 6.66 (s, 1H), 4.67 (s, 2H);

LCMS (ESI⁺) M+H⁺: 412.

### Example 279

### 6-(1-(4-Fluoro-2-methylphenyl)-3-(trifluoromethyl)-1H-pyrazol-5-yl)-8-(hydroxymethyl)-2H-benzo[b][1,4]oxazin-3(4H)-one

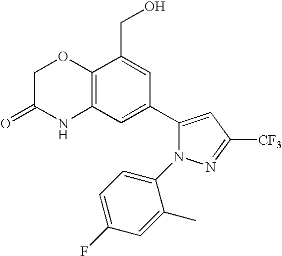

### Methyl 2-hydroxy-3-nitrobenzoate

To a stirred solution of 2-hydroxy-3-nitrobenzoic acid (25.0 g, 136.5 mmol) in MeOH (273.0 mL, 136.5 mmol) was slowly added SOCl₂ (12.45 mL, 170.7 mmol) and the mixture was refluxed for 24 hr and concentrated in vacuo to give the title compound as a white solid (25.0 g, 93% yield).

LCMS (ESI⁻) M−H⁻: 196.

### Methyl 5-bromo-2-hydroxy-3-nitrobenzoate

To a stirred solution of methyl 2-hydroxy-3-nitrobenzoate (9.25 g, 46.92 mmol) in acetic acid (156.4 mL, 46.92 mmol) at room temperature was added bromine (3.61 mL, 70.38 mmol) and stirring was continued overnight. The reaction mixture was poured into water and the precipitated solid was collected by vacuum filtration, washed with ether/petroleum ether and dried in a vacuum oven to give the title compound (13.0 g, 99%).

LCMS (ESI⁻) M−H⁻: 274, 276.

### Methyl 5-bromo-2-(2-methoxy-2-oxoethoxy)-3-nitrobenzoate

According to the method of Example 108, methyl 5-bromo-2-hydroxy-3-nitrobenzoate (13.0 g, 47.09 mmol) and methyl 2-bromoacetate (14.41 g, 94.19 mmol) were reacted at room temperature to give the title compound as a brown oil (12.0 g, 73%).

LCMS (ESI⁻) (M-CH₂CO₂Me)⁻: 274, 276.

### Methyl 6-bromo-3-oxo-3,4-dihydro-2H-benzo[b][1,4]oxazine-8-carboxylate

According to the method of Example 108, methyl 5-bromo-2-(2-methoxy-2-oxoethoxy)-3-nitrobenzoate (16.50 g, 47.40 mmol) gave the title compound as a white solid (7.22 g, 53%).

LCMS (ESI⁻) M−H⁻: 284, 286.

### Methyl 6-(1-(4-fluoro-2-methylphenyl)-3-(trifluoromethyl)-1H-pyrazol-5-yl)-3-oxo-3,4-dihydro-2H-benzo[b][1,4]oxazine-8-carboxylate

According to the method of Example 250, methyl 6-bromo-3-oxo-3,4-dihydro-2H-benzo[b][1,4]oxazine-8-carboxylate (1.00 g, 3.50 mmol) and 1-(4-fluoro-2-methylphenyl)-3-(trifluoromethyl)-1H-pyrazol-5-ylboronic acid (1.01 g, 3.50 mmol) were reacted to give the title compound as a pale yellow solid (1.10 g, 70%).

¹H-NMR (400 MHz, DMSO-d₆) δ: 10.95 (s, 1H), 7.46 (dd, J=8.6, 5.5 Hz, 1H), 7.31 (dd, J=9.8, 3.2 Hz, 1H), 7.27 (d, J=2.3 Hz, 1H), 7.24 (s, 1H), 7.20 (td, J=8.2, 2.7 Hz, 1H), 6.84 (d, J=2.3 Hz, 1H), 4.67 (s, 2H), 3.76 (s, 3H), 1.91 (s, 3H)

### 6-(1-(4-Fluoro-2-methylphenyl)-3-(trifluoromethyl)-1H-pyrazol-5-yl)-8-(hydroxymethyl)-2H-benzo[b][1,4]oxazin-3(4H)-one

To a stirred solution of methyl 6-(1-(4-fluoro-2-methylphenyl)-3-(trifluoromethyl)-1H-pyrazol-5-yl)-3-oxo-3,4-dihydro-2H-benzo[b][1,4]oxazine-8-carboxylate (112 mg, 0.25 mmol) in THF (25 mL) was added dropwise lithium aluminum hydride (0.25 mL, 1.0 M in THF, 0.25 mmol) in THF and stirring was continued overnight. The reaction mixture was poured into water, extracted three times with EtOAc, and the organic layer was washed with brine, dried (MgSO₄) and concentrated in vacuo. The residue was triturated with ether/petroleum ether and filtered to give the title compound as a white solid (7 mg, 7%).

¹H-NMR (400 MHz, DMSO-d₆) δ: 7.41 (dd, J=8.6, 5.5 Hz, 1H), 7.28 (dd, J=9.4, 2.9 Hz, 1H), 7.18 (ddd, J=9.4, 8.6, 2.9 Hz, 1H), 7.09 (s, 1H), 7.01 (d, J=2.0 Hz, 1H), 6.56 (d, J=2.0 Hz, 1H), 4.56 (s, 2H), 4.41 (s, 2H), 1.91 (s, 3H); LCMS (ESI⁻) M−H⁻: 420.

### Example 280

### 7-Bromo-6-(1-(4-fluorophenyl)-3-(trifluoromethyl)-1H-pyrazol-5-yl)-2H-benzo[b][1,4]oxazin-3(4H)-one

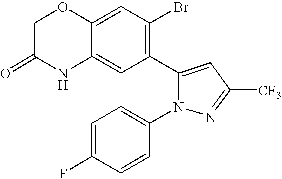

According to the method of Example 105, 6-(1-(4-fluorophenyl)-3-(trifluoromethyl)-1H-pyrazol-5-yl)-2H-benzo[b][1,4]oxazin-3(4H)-one (100 mg, 0.27 mmol), NBS (59 mg, 0.33 mmol) gave the title compound, as a white solid (29 mg, 24%) after flash chromatography on silica gel (10-30% EtOAc in petroleum ether).

¹H-NMR (400 MHz, CD₂Cl₂) δ: 7.70 (brs, 1H), 7.21 (m, 2H), 7.15 (s, 1H), 6.98 (m, 2H), 6.66 (s, 1H), 6.63 (s, 1H), 4.57 (s, 2H); LCMS (ESI⁻) M−H: 454, 456.

### Example 281

### 6-(1-(4-Fluorophenyl)-3-(trifluoromethyl)-1H-pyrazol-5-yl)-7-nitro-2H-benzo[b][1,4]oxazin-3(4H)-one

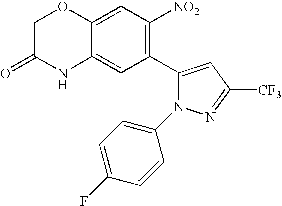

To a stirred solution of 6-(1-(4-fluorophenyl)-3-(trifluoromethyl)-1H-pyrazol-5-yl)-2H-benzo[b][1,4]oxazin-3(4H)-one (1.00 g, 2.65 mmol) in acetic acid (20 mL) at room temperature was added nitric acid (0.84 g, 13.25 mmol) and stirring was continued for 24 hr. The reaction mixture was poured into water and the solid precipitate was collected by vacuum filtration and dried to give the title compound as a tan solid (0.72 g, 64%).

¹H-NMR (400 MHz, DMSO-d₆) δ: 11.39 (s, 1H), 7.70 (s, 1H), 7.34 (m, 2H), 7.28 (m, 2H), 7.16 (s, 1H), 7.02 (s, 1H), 4.79 (s, 2H); LCMS (ESI⁻) M−H⁻: 421.

### Example 282

### 7-Amino-6-(1-(4-fluorophenyl)-3-(trifluoromethyl)-1H-pyrazol-5-yl)-2H-benzo[b][1,4]oxazin-3(4H)-one

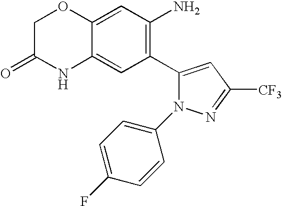

To a stirred solution of 6-(1-(4-fluorophenyl)-3-(trifluoromethyl)-1H-pyrazol-5-yl)-7-nitro-2H-benzo[b][1,4]oxazin-3(4H)-one (500 mg, 1.18 mmol) in acetic acid (10 mL) was added portion-wise zinc dust (387 mg, 5.90 mmol) and stirring was continued for 24 hr. The reaction mixture was filtered to remove zinc. Addition of cold water to the filtrate precipitated material which was filtered and dried to give the title compound as a gray solid (300 mg, 65%).

¹H-NMR (400 MHz, DMSO-d₆) δ: 10.32 (s, 1H), 7.43 (m, 2H), 7.30 (m, 2H), 6.94 (s, 1H), 6.38 (s, 1H), 6.31 (s, 1H), 4.47 (s, 2H); LCMS (ESI⁻) M−H⁻: 391.

### Example 283

### 6-(4-(4-Fluorophenyl)-1,3-dimethyl-1H-pyrazol-5-yl)-2H-benzo[b][1,4]oxazin-3(4H)-one

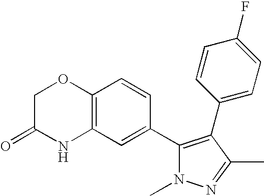

### 6-(4-Bromo-1,3-dimethyl-1H-pyrazol-5-yl)-2H-benzo[b][1,4]oxazin-3(4H)-one

According to the method of Example 270, 6-(1,3-dimethyl-1H-pyrazol-5-yl)-2H-benzo[b][1,4]oxazin-3(4H)-one (1.0 g, 4.1 mmol) was reacted with NBS (0.73 g, 4.1 mmol) to give the title compound as a white solid (1.3 g, 98%).

LCMS (ESI⁺) M+H⁺: 324, 326.

### 6-(4-(4-Fluorophenyl)-1,3-dimethyl-1H-pyrazol-5-yl)-2H-benzo[b][1,4]oxazin-3(4H)-one

A mixture of 6-(4-bromo-1,3-dimethyl-1H-pyrazol-5-yl)-2H-benzo[b][1,4]oxazin-3(4H)-one (100 mg, 0.31 mmol, 4-fluorophenylboronic acid (86.9 mg, 0.62 mmol) and 2M K₃PO₄ (1.47 mL, 2.95 mmol) in dioxane (3 mL) was degassed with N₂ for 15 min. Pd(PPh₃)₄ (17.9 mg, 0.016 mmol) was added, and the mixture was heated to 90° C. for 16 hr. The mixture was poured into saturated NaHCO₃, extracted with DCM, and the organic layer was washed with brine, dried (MgSO₄), and concentrated in vacuo. Preparative RP-HPLC of the residue gave the title compound as a white solid (17.8 mg, 17%).

¹H-NMR (400 MHz, CDCl₃) δ: 7.84 (brs, 1H), 7.00 (m, 5H), 6.85 (dd, J=8.2, 2.0 Hz, 1H), 6.57 (d, J=2.0 Hz, 1H), 4.65 (s, 2H), 3.75 (s, 3H), 2.29 (s, 3H); LCMS (ESI⁺) M+H⁺: 338.

### Example 284

### 6-(4-(4-Fluoro-2-methylphenyl)-1,3-dimethyl-1H-pyrazol-5-yl)-2H-benzo[b][1,4]oxazin-3(4H)-one

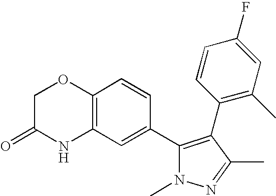

According to the method of Example 283, 6-(4-bromo-1,3-dimethyl-1H-pyrazol-5-yl)-2H-benzo[b][1,4]oxazin-3(4H)-one (100 mg, 0.31 mmol) and 4-fluoro-2-methylphenylboronic acid (95.6 mg, 0.62 mmol) were reacted to give the title compound as a white solid (5.6 mg, 5.1%).

¹H-NMR (400 MHz, CDCl₃) δ: 7.52 (brs, 1H), 7.01 (m, 1H), 6.95 (d, J=8.2 Hz, 1H), 6.84 (m, 2H), 6.78 (m, 1H), 6.47 (m, 1H), 4.62 (s, 2H), 3.81 (s, 3H), 2.10 (s, 3H), 1.96 (s, 3H); LCMS (ESI⁺) M+H⁺: 352.

### Example 285

### 6-(1-Methyl-4-phenyl-3-(trifluoromethyl)-1H-pyrazol-5-yl)-2H-benzo[b][1,4]oxazin-3(4H)-one

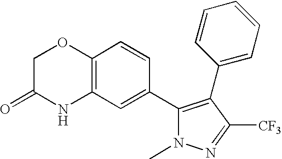

### 6-(1-Methyl-3-(trifluoromethyl)-1H-pyrazol-5-yl)-2H-benzo[b][1,4]oxazin-3(4H)-one

According to the method of Example 71, 4,4,4-trifluoro-1-(3-oxo-3,4-dihydro-2H-benzo[b][1,4]oxazin-6-yl)butane-1,3-dione (500 mg, 1.74 mmol) and 1-methylhydrazine (96 μL, 1.83 mmol) were reacted to give the title compound as a white solid (460 mg, 88%).

LCMS (ESI⁺) M+H⁺: 298.

### 6-(4-Bromo-1-methyl-3-(trifluoromethyl)-1H-pyrazol-5-yl)-2H-benzo[b][1,4]oxazin-3(4H)-one

According to the method of Example 270, 6-(1-methyl-3-(trifluoromethyl)-1H-pyrazol-5-yl)-2H-benzo[b][1,4]oxazin-3(4H)-one (460 mg, 1.55 mmol) and NBS (275 mg, 1.55 mmol) were reacted to give the title compound as a white solid (500 mg, 86%).

LCMS (ESI⁺): 376, 378 (M+H⁺).

### 6-(1-Methyl-4-phenyl-3-(trifluoromethyl)-1H-pyrazol-5-yl)-2H-benzo[b][1,4]oxazin-3(4H)-one

According to the method of Example 283, 6-(4-bromo-1-methyl-3-(trifluoromethyl)-1H-pyrazol-5-yl)-2H-benzo[b][1,4]oxazin-3(4H)-one (100 mg, 0.27 mmol) and phenylboronic acid (64.8 mg, 0.53 mmol) were reacted to give the title compound as a white solid (21 mg, 21%).

¹H-NMR (400 MHz, CDCl₃) δ: 7.91 (brs, 1H), 7.27 (m, 3H), 7.15 (m, 2H), 6.99 (d, J=8.2 Hz, 1H), 6.86 (dd, J=8.2, 2.0 Hz, 1H), 6.56 (d, J=2.0 Hz, 1H), 4.64 (s, 2H), 3.86 (s, 3H); LCMS (ESI⁺) M+H⁺: 374.

### Example 286

### 6-(4-(4-Fluorophenyl)-1-methyl-3-(trifluoromethyl)-1H-pyrazol-5-yl)-2H-benzo[b][1,4]oxazin-3(4H)-one

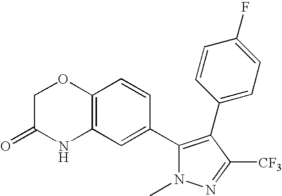

According to the method of Example 283, 6-(4-bromo-1-methyl-3-(trifluoromethyl)-1H-pyrazol-5-yl)-2H-benzo[b][1,4]oxazin-3(4H)-one (100 mg, 0.27 mmol) and 4-fluorophenylboronic acid (74.4 mg, 0.53 mmol) were reacted to give the title compound as a white solid (32 mg, 31%).

¹H-NMR (400 MHz, CDCl₃) δ: 8.23 (brs, 1H), 7.11 (m, 2H), 6.97 (m, 3H), 6.84 (dd, J=8.6, 2.0 Hz, 1H), 6.57 (d, J=2.0 Hz, 1H), 4.66 (s, 2H) 3.86 (s, 3H); LCMS (ESI⁺) M+H⁺: 392.

### Example 287

### 6-(1,3-Dimethyl-4-p-tolyl-1H-pyrazol-5-yl)-2H-benzo[b][1,4]oxazin-3(4H)-one

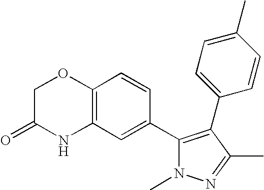

To a stirred mixture of 6-(4-bromo-1,3-dimethyl-1H-pyrazol-5-yl)-2H-benzo[b][1,4]oxazin-3(4H)-one (100 mg, 0.31 mmol), p-tolylboronic acid (127 mg, 0.93 mmol), and 2M K₃PO₄ (1.47 mL, 2.95 mmol) in degassed dioxane (4 mL) was added bis(tri-t-butylphosphine)palladium (0) (7.93 mg, 0.016 mmol) and the mixture was heated at 90° C. for 16 hr. The reaction mixture was cooled, filtered through magnesium sulfate and concentrated in vacuo. Flash chromatography of the residue on silica gel (0-2% MeOH in DCM) followed by trituration with hexanes gave the title compound as a white solid (57.7 mg, 56%).

¹H-NMR (400 MHz, CDCl₃) δ: 7.79 (brs, 1H), 7.07 (d, J=8.2 Hz, 2H), 6.98 (m, 3H), 6.87 (dd, J=8.2, 2.0 Hz, 1H), 6.58 (d, J=2.0 Hz, 1H), 4.64 (s, 2H), 3.75 (s, 3H), 2.32 (s, 3H), 2.31 (s, 3H); LCMS (ESI⁺) M+H⁺: 334.

### Example 288

### 6-(1,3-Dimethyl-4-phenyl-1H-pyrazol-5-yl)-2H-benzo[b][1,4]oxazin-3(4H)-one

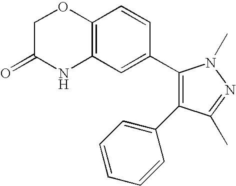

### 1-(3-Oxo-3,4-dihydro-2H-benzo[b][1,4]oxazin-6-yl)butane-1,3-dione

To a stirred mixture of 60% NaH (4.18 g, 105 mmol) in THF (50 mL) was carefully added EtOAc (10.2 mL, 105 mmol). To this resulting mixture were added sequentially 6-acetyl-2H-benzo[b][1,4]oxazin-3(4H)-one (5.0 g, 26.2 mmol) ethanol (a few drops) and a solution of [2,4]-dibenzo-18-crown-6 (111 mg, 0.418 mmol) in THF (50 mL). The mixture was refluxed for 16 hr, cooled, and partitioned between 10% H₂SO₄ and EtOAc. The organic layer was separated and washed with water, 5% aqueous Na₂CO₃, water and brine, dried (Mg₂SO₄), and concentrated in vacuo. The residue was triturated with ether and filtered to give the title compound as an off-white solid (4.36 g, 72%).

¹H-NMR (400 MHz, CDCl₃) δ: 8.82 (brs, 1H), 7.52 (dd, J=8.4, 2.0 Hz, 1H), 7.42 (d, J=2.0 Hz, 1H), 7.12 (d, J=8.4 Hz, 1H), 6.10 (s, 1H), 4.71 (s, 2H), 2.19 (s, 3H); LCMS (ESI⁻): M−H⁻: 232.

### 6-(1,3-Dimethyl-1H-pyrazol-5-yl)-2H-benzo[b][1,4]oxazin-3(4H)-one

To a stirred mixture of 1-methylhydrazine (0.88 mL, 16.66 mmol) in anhydrous IPA (80 mL) was added TFA (2.61 mL, 34.11 mmol) and stirring was continued for 15 min. 1-(3-Oxo-3,4-dihydro-2H-benzo[b][1,4]oxazin-6-yl)butane-1,3-dione (3.7 g, 15.86 mmol) was added and the mixture was heated at 60° C. for 16 hr. Most of the IPA was removed in vacuo, water was added and the mixture was basified to pH 5-6 with 1M NaOH. The resulting solid was collected by filtration, washed with petroleum ether and purified by flash chromatography on silica gel (0-3% MeOH in DCM) to give the title compound as a white solid (1.0 g, 26%).

¹H-NMR (400 MHz, CDCl₃) δ: 7.95 (brs, 1H), 7.05 (d, J=8.2 Hz, 1H), 7.01 (dd, J=8.2, 2.0 Hz, 1H), 6.81 (d, J=2.0 Hz, 1H), 6.04 (s, 1H), 4.67 (s, 2H), 3.79 (s, 3H), 2.29 (s, 3H); LCMS (ESI⁺), M+H⁺: 244.

### 6-(4-Bromo-1,3-dimethyl-1H-pyrazol-5-yl)-2H-benzo[b][1,4]oxazin-3(4H)-one

To a stirred solution of 6-(1,3-dimethyl-1H-pyrazol-5-yl)-2H-benzo[b][1,4]oxazin-3(4H)-one (1.0 g, 4.1 mmol) in DMF (4 mL) was added NBS (0.73 g, 4.1 mmol) and stirring was continued for 2 hr. The mixture was diluted with water and cooled to 0° C. for 10 min. The solid was filtered, washed with water and petroleum ether, and dried to give the title compound as a white solid (1.3 g, 98%).

¹H-NMR (400 MHz, CDCl₃) δ: 8.23 (brs, 1H), 7.10 (d, J=8.2 Hz, 1H), 7.00 (dd, J=8.2, 2.0 Hz, 1H), 6.84 (d, J=2.0 Hz, 1H), 4.70 (s, 2H), 3.75 (s, 3H), 2.28 (s, 3H); LCMS (ESI⁺), M+H⁺: 322, 324.

### 6-(1,3-Dimethyl-4-phenyl-1H-pyrazol-5-yl)-2H-benzo[b][1,4]oxazin-3(4H)-one

A mixture of 6-(4-bromo-1,3-dimethyl-1H-pyrazol-5-yl)-2H-benzo[b][1,4]oxazin-3(4H)-one (100 mg, 0.31 mmol), phenylboronic acid (114 mg, 0.93 mmol) and aqueous 2.0 M K₃PO₄ (1.47 mL, 2.95 mmol) in dioxane (4 mL) was degassed (N₂), and bis(tri-t-butylphosphine)palladium (0) (7.93 mg, 0.016 mmol) was added. The mixture was heated at 90° C. for 16 hr, dried (Na₂SO₄), and concentrated in vacuo. Flash chromatography of the residue on silica gel (0-2% MeOH in DCM) and trituration with hexanes gave the title compound as a light yellow solid (67.7 mg, 68%).

¹H-NMR (400 MHz, CDCl₃) δ: 8.28 (brs, 1H), 7.26 (m, 2H), 7.19 (m, 1H), 7.08 (m, 2H), 6.97 (d, J=8.2 Hz, 1H), 6.86 (dd, J=8.2, 2.0 Hz, 1H), 6.61 (d, J=2.0 Hz, 1H), 4.64 (s, 2H), 3.75 (s, 3H), 2.31 (s, 3H), LCMS (ESI⁺), M+H⁺: 320.

### Example 289

### 6-(1,3-Dimethyl-4-(4-(trifluoromethyl)phenyl)-1H-pyrazol-5-yl)-2H-benzo[b][1,4]oxazin-3(4H)-one

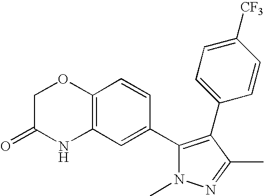

According to the method of Example 287, 6-(4-bromo-1,3-dimethyl-1H-pyrazol-5-yl)-2H-benzo[b][1,4]oxazin-3(4H)-one (100 mg, 0.31 mmol) and 4-(trifluoromethyl)phenylboronic acid (177 mg, 0.93 mmol) were reacted to give the title compound as a white solid (54 mg, 45%).

¹H-NMR (400 MHz, CDCl₃) δ: 8.01 (brs, 1H), 7.51 (d, J=8.2 Hz, 2H), 7.18 (d, J=8.2 Hz, 2H), 7.01 (d, J=8.2 Hz 1H), 6.85 (dd, J=8.2, 2.0 Hz, 1H), 6.59 (d, J=2.0 Hz, 1H), 4.66 (s, 2H), 3.75 (s, 3H), 2.33 (s, 3H); LCMS (ESI⁺) M+H⁺: 388.

### Example 290

### 6-(4-(4-Methoxyphenyl)-1,3-dimethyl-1H-pyrazol-5-yl)-2H-benzo[b][1,4]oxazin-3(4H)-one

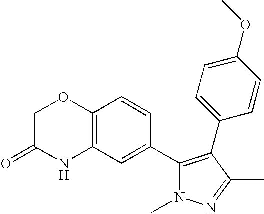

According to the method of Example 287, 6-(4-bromo-1,3-dimethyl-1H-pyrazol-5-yl)-2H-benzo[b][1,4]oxazin-3(4H)-one (100 mg, 0.31 mmol) and 4-methoxyphenylboronic acid (142 mg, 0.93 mmol) were reacted to give the title compound as a white solid (77 mg, 71%).

¹H-NMR (400 MHz, CDCl₃) δ: 7.58 (brs, 1H), 6.99 (m, 3H), 6.87 (dd, J=8.6, 2.0 Hz, 1H), 6.81 (m, 2H), 6.56 (d, J=2.0 Hz, 1H), 4.64 (s, 2H), 3.79 (s, 3H), 3.75 (s, 3H), 2.29 (s, 3H);

LCMS (ESI⁺) M+H⁺: 350.

### Example 291

### 6-(4-(4-Hydroxyphenyl)-1,3-dimethyl-1H-pyrazol-5-yl)-2H-benzo[b][1,4]oxazin-3(4H)-one

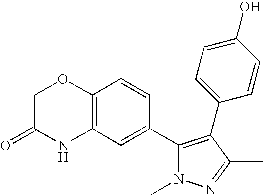

To a stirred solution of 6-(4-(4-methoxyphenyl)-1,3-dimethyl-1H-pyrazol-5-yl)-2H-benzo[b][1,4]oxazin-3(4H)-one (22 mg, 0.06 mmol) in DCM at 0° C. was added drop-wise BBr₃ (63 μL, 0.06 mmol) and the mixture was allowed to warm to room temperature over night. The mixture was quenched with saturated NH₄Cl, extracted with EtOAc, and the organic layer was washed with brine, dried (MgSO₄), and concentrated in vacuo. Flash chromatography of the residue on silica gel (0-5% MeOH in DCM) gave the title compound as a white solid (4.2 mg, 20%).

¹H-NMR (400 MHz, CDCl₃) δ: 7.48 (brs, 1H), 7.00 (d, J=8.2 Hz, 1H), 6.96 (d, J=8.2 Hz, 2H), 6.87 (dd, J=8.2, 2.0 Hz, 1H), 6.74 (d, J=8.2 Hz, 2H), 6.55 (d, J=2.0 Hz, 1H), 4.78 (brs, 1H), 4.65 (s, 2H), 3.75 (s, 3H), 2.29 (s, 3H); LCMS (ESI⁺) (M+H⁺): 336.

### Example 292

### 6-(4-(4-Fluoro-3-methylphenyl)-1,3-dimethyl-1H-pyrazol-5-yl)-2H-benzo[b][1,4]oxazin-3(4H)-one

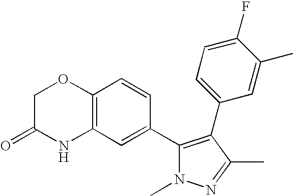

According to the method of Example 287, 6-(4-bromo-1,3-dimethyl-1H-pyrazol-5-yl)-2H-benzo[b][1,4]oxazin-3(4H)-one (100 mg, 0.31 mmol) and 4-fluoro-3-methylphenylboronic acid (143 mg, 0.93 mmol) were reacted to give the title compound as a white solid (70 mg, 64%).

¹H-NMR (400 MHz, CDCl₃) δ: 7.61 (brs, 1H), 6.99 (d, J=8.2 Hz, 1H), 6.91 (dd, J=8.2, 2.0 Hz, 1H), 6.86 (d, J=9.8 Hz, 1H), 6.86 (dd, J=8.2, 2.0 Hz, 1H), 6.81 (m, 1H), 6.55 (d, J=2.0 Hz, 1H), 4.65 (s, 2H), 3.75 (s, 3H), 2.28 (s, 3H), 2.21 (d, J=2.0 Hz, 3H); LCMS (ESI⁺) M+H⁺: 352.

### Example 293

### 6-(1,3-Dimethyl-4-(3-(trifluoromethyl)phenyl)-1H-pyrazol-5-yl)-2H-benzo[b][1,4]oxazin-3(4H)-one

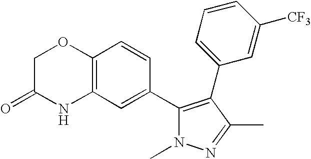

According to the method of Example 287, 6-(4-bromo-1,3-dimethyl-1H-pyrazol-5-yl)-2H-benzo[b][1,4]oxazin-3(4H)-one (79 mg, 0.25 mmol) and 3-(trifluoromethyl)phenylboronic acid (140 mg, 0.74 mmol) were reacted to give the title compound as a white solid (12 mg, 12%).

¹H-NMR (400 MHz, CDCl₃) δ: 7.75 (s, 1H), 7.44 (d, 1H), 7.36 (m, 2H), 7.23 (m, 1H), 7.01 (d, J=8.2 Hz, 1H), 7.86 (dd, J=8.2, 2.0 Hz, 1H), 6.57 (d, J=2.0 Hz, 1H), 4.64 (s, 2H), 3.76 (s, 3H), 2.33 (s, 3H); LCMS (ESI⁺) M+H⁺: 388.

### Example 294

### 6-(4-(3,4-Dichlorophenyl)-1,3-dimethyl-1H-pyrazol-5-yl)-2H-benzo[b][1,4]oxazin-3(4H)-one

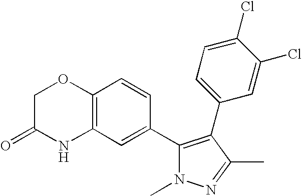

According to the method of Example 287, 6-(4-bromo-1,3-dimethyl-1H-pyrazol-5-yl)-2H-benzo[b][1,4]oxazin-3(4H)-one (90 mg, 0.28 mmol) and 3,4-dichlorophenylboronic acid (160 mg, 0.84 mmol) were reacted to give the title compound as a white solid (7 mg, 7%).

¹H-NMR (400 MHz, CDCl₃) δ: 7.70 (brs, 1H), 7.31 (dd, J=8.6, 2.0 Hz, 1H), 7.20 (t, J=2.0 Hz, 1H), 7.02 (dd, J=8.6, 2.0 Hz, 1H) 6.86 (m, 2H), 6.57 (t, J=2.0 Hz, 1H), 4.67 (s, 2H), 3.74 (s, 3H), 2.31 (s, 3H); LCMS (ESI⁺) M+H⁺: 388.

### Example 295

### 6-(4-(4-Chlorophenyl)-1,3-dimethyl-1H-pyrazol-5-yl)-2H-benzo[b][1,4]oxazin-3(4H)-one

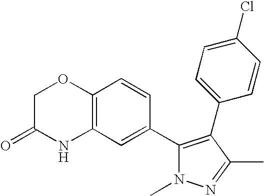

According to the method of Example 287, 6-(4-bromo-1,3-dimethyl-1H-pyrazol-5-yl)-2H-benzo[b][1,4]oxazin-3(4H)-one (88 mg, 0.27 mmol) and 4-chlorophenylboronic acid (128 mg, 0.82 mmol) were reacted to give the title compound as a white solid (10 mg, 10%).

¹H-NMR (400 MHz, CDCl₃) δ: 7.74 (brs, 1H), 7.23 (m, 2H), 7.00 (m, 3H), 6.85 (dd, J=8.2, 2.0 Hz, 1H), 6.56 (d, J=2.0 Hz, 1H), 4.66 (s, 2H), 3.75 (s, 3H), 2.30 (s, 3H); LCMS (ESI⁺) M+H⁺: 354.

### Example 296

### 6-(1-Ethyl-4-(4-fluorophenyl)-3-methyl-1H-pyrazol-5-yl)-2H-benzo[b][1,4]oxazin-3(4H)-one

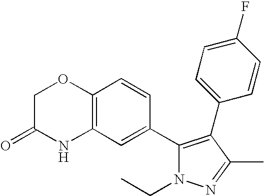

### 6-(1-Ethyl-3-methyl-1H-pyrazol-5-yl)-2H-benzo[b][1,4]oxazin-3(4H)-one

According to the method of Example 71, 1-(3-oxo-3,4-dihydro-2H-benzo[b][1,4]oxazin-6-yl)butane-1,3-dione (1.0 g, 4.29 mmol) and ethylhydrazine oxalate (676 mg, 4.50 mmol) were reacted to give the title compound as a white solid (1.1 g, 76%).

LCMS (ESI⁺) M+H⁺: 258.

### 6-(1-Ethyl-4-iodo-3-methyl-1H-pyrazol-5-yl)-2H-benzo[b][1,4]oxazin-3(4H)-one

According to the method of Example 71, 6-(1-ethyl-3-methyl-1H-pyrazol-5-yl)-2H-benzo[b][1,4]oxazin-3(4H)-one (838 mg, 3.26 mmol) and NIS (733 mg, 3.26 mmol) were reacted to give the title compound as a white solid (1.1 g, 91%).

LCMS (ESI⁺) M+H⁺: 384.

### 6-(1-Ethyl-4-(4-fluorophenyl)-3-methyl-1H-pyrazol-5-yl)-2H-benzo[b][1,4]oxazin-3(4H)-one

According to the method of Example 287, 6-(1-ethyl-4-iodo-3-methyl-1H-pyrazol-5-yl)-2H-benzo[b][1,4]oxazin-3(4H)-one (100 mg, 0.26=mol) and 4-fluorophenylboronic acid (110 mg, 0.78 mmol) were reacted to give the title compound as a light yellow solid (23 mg, 25%).

¹H-NMR (400 MHz, CDCl₃) δ: 8.04 (brs, 1H), 7.03 (m, 2H), 6.99 (d, J=8.2 Hz, 1H), 6.94 (m, 2H), 6.85 (dd, J=2.0, 8.2 Hz, 1H), 6.58 (d, J=2.0 Hz, 1H), 4.65 (s, 2H), 4.02 (q, J=7.2 Hz, 2H), 2.31 (s, 3H), 1.37 (t, J=7.2 Hz 3H); (ESI⁺) M+H⁺: 352.

### Example 297

### 6-(4-(4-Fluorophenyl)-1-isopropyl-3-methyl-1H-pyrazol-5-yl)-2H-benzo[b][1,4]oxazin-3(4H)-one

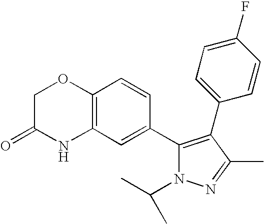

### 6-(1-Isopropyl-3-methyl-1H-pyrazol-5-yl)-2H-benzo[b][1,4]oxazin-3(4H)-one

According to the method of Example 71, 1-(3-oxo-3,4-dihydro-2H-benzo[b][1,4]oxazin-6-yl)butane-1,3-dione (1.0 g, 4.29 mmol) and 1-isopropylhydrazine hydrochloride (498 mg, 4.50 mmol) were reacted to give the title compound as a white solid (1.1 g, 93%).

LCMS (ESI⁺) M+H⁺: 272.

### 6-(4-Iodo-1-isopropyl-3-methyl-1H-pyrazol-5-yl)-2H-benzo[b][1,4]oxazin-3(4H)-one

According to the method of Example 59, 6-(1-isopropyl-3-methyl-1H-pyrazol-5-yl)-2H-benzo[b][1,4]oxazin-3(4H)-one (1.08 g, 3.98 mmol) and NIS (896 mg, 3.98 mmol) were reacted to give the title compound as a white solid (1.5 g, 94%).

LCMS (ESI⁺) M+H⁺: 398.

### 6-(4-(4-Fluorophenyl)-1-isopropyl-3-methyl-1H-pyrazol-5-yl)-2H-benzo[b][1,4]oxazin-3(4H)-one

According to the method of Example 73, 6-(4-iodo-1-isopropyl-3-methyl-1H-pyrazol-5-yl)-2H-benzo[b][1,4]oxazin-3(4H)-one (100 mg, 0.25 mmol) and 4-fluorophenylboronic acid (106 mg, 0.65 mmol) were reacted to give the title compound as a white solid (22 mg, 24%).

¹H-NMR (400 MHz, CDCl₃) δ: 7.76 (brs, 1H), 7.03 (m, 3H), 6.94 (m, 2H), 6.83 (dd, J=8.2, 2.0 Hz, 1H), 6.55 (d, J=2.0 Hz 1H), 4.65 (s, 2H), 4.34 (m, 1H), 2.32 (s, 3H), 1.46 (d, J=6.6 Hz, 6H); LCMS (ESI⁺) M+H⁺: 366.

### Example 298

### 6-(4-(4-Fluorophenyl)-3-methyl-1-(2,2,2-trifluoroethyl)-1H-pyrazol-5-yl)-2H-benzo[b][1,4]oxazin-3(4H)-one

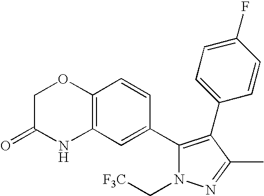

### 6-(3-Methyl-1-(2,2,2-trifluoroethyl)-1H-pyrazol-5-yl)-2H-benzo[b][1,4]oxazin-3(4H)-one

According to the method of Example 71, 1-(3-oxo-3,4-dihydro-2H-benzo[b][1,4]oxazin-6-yl)butane-1,3-dione (1.0 g, 4.29 mmol) and 1-(2,2,2-trifluoroethyl)hydrazine (734 mg, 4.50 mmol) were reacted to give the title compound as a white solid (1.0 g, 78%).

LCMS (ESI⁺) M+H⁺: 312.

### 6-(4-Iodo-3-methyl-1-(2,2,2-trifluoroethyl)-1H-pyrazol-5-yl)-2H-benzo[b][1,4]oxazin-3(4H)-one

According to the method of Example 59, 6-(3-methyl-1-(2,2,2-trifluoroethyl)-1H-pyrazol-5-yl)-2H-benzo[b][1,4]oxazin-3(4H)-one (1.04 g, 3.34 mmol) and N-iodosuccinimide (752 mg, 3.34 mmol) were reacted to give the title compound as a white solid (1.4 g, 95%).

LCMS (ESI⁺) M+H⁺: 438.

### 6-(4-(4-Fluorophenyl)-3-methyl-1-(2,2,2-trifluoroethyl)-1H-pyrazol-5-yl)-2H-benzo[b][1,4]oxazin-3(4H)-one

According to the method of Example 73, 6-(4-iodo-3-methyl-1-(2,2,2-trifluoroethyl)-1H-pyrazol-5-yl)-2H-benzo[b][1,4]oxazin-3(4H)-one (100 mg, 0.23 mmol) and 4-fluorophenylboronic acid (96.0 mg, 0.69 mmol) were reacted to give the title compound as a white solid (23 mg, 25%).

¹H-NMR (400 MHz, CDCl₃) δ: 7.76 (brs, 1H), 7.05 (m, 2H), 7.02 (d, J=8.2 Hz, 2H), 6.96 (m, 2H), 6.85 (dd, J=8.2, 2.0 Hz, 1H), 6.57 (d, J=2.0 Hz, 1H), 4.66 (s, 2H), 4.54 (q, J=8.1 Hz, 2H), 2.31 (s, 3H); LCMS (ESI⁺) M+H⁺: 406.

### Example 299

### 6-(4-(4-Fluorophenyl)-3-methyl-1H-pyrazol-5-yl)-2H-benzo[b][1,4]oxazin-3(4H)-one

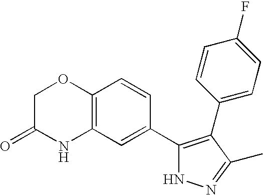

### 6-(3-Methyl-1H-pyrazol-5-yl)-2H-benzo[b][1,4]oxazin-3(4H)-one

According to the method of Example 71, 1-(3-oxo-3,4-dihydro-2H-benzo[b][1,4]oxazin-6-yl)butane-1,3-dione (500 mg, 2.14 mmol) and hydrazine (71 mL, 2.25 mmol) were reacted to give the title compound as a white solid 380 mg, 77%).

LCMS (ESI⁺) M+H⁺: 230.

### 6-(4-Bromo-3-methyl-1H-pyrazol-5-yl)-2H-benzo[b][1,4]oxazin-3(4H)-one

According to the method of Example 56, 6-(3-methyl-1H-pyrazol-5-yl)-2H-benzo[b][1,4]oxazin-3(4H)-one (380 mg, 1.66 mmol) and NBS (295 mg, 1.66 mmol) were reacted to give the title compound as a white solid (0.480 g, 94%).

LCMS (ESI⁺) M+H⁺: 308, 310.

### 6-(4-(4-Fluorophenyl)-3-methyl-1H-pyrazol-5-yl)-2H-benzo[b][1,4]oxazin-3(4H)-one

According to the method of Example 73, 6-(4-bromo-3-methyl-1H-pyrazol-5-yl)-2H-benzo[b][1,4]oxazin-3(4H)-one (158 mg, 0.51 mmol) and 4-fluorophenylboronic acid (215 mg, 1.54 mmol) were reacted to give the title compound as an off-white solid (88 mg, 54%).

¹H-NMR (400 MHz, CDCl₃) δ: 10.56 (brs, 1H), 7.52 (s, 1H), 7.26 (s, 1H), 7.19 (m, 2H), 7.08 (m, 2H), 6.78 (d, J=2.0 Hz, 1H), 6.72 (dd, J=8.6, 2.0 Hz, 1H), 4.69 (s, 2H), 2.30 (s, 3H);

LCMS (ESI⁺) M+H⁺: 324.

### Example 300

### 6-(4-(4-Fluorophenyl)-1-methyl-1H-pyrazol-5-yl)-2H-benzo[b][1,4]oxazin-3(4H)-one

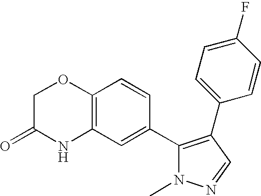

### 6-(4-Bromo-1-methyl-1H-pyrazol-5-yl)-2H-benzo[b][1,4]oxazin-3(4H)-one

According to the method of Example 56, 6-(1-methyl-1H-pyrazol-5-yl)-2H-benzo[b][1,4]oxazin-3(4H)-one (1.26 g, 5.50 mmol) and NBS (0.978 g, 5.50 mmol) were reacted to give the title compound as a white solid (1.1 g, 67%).

LCMS (ESI⁺) M+H⁺: 308, 310.

### 6-(4-(4-Fluorophenyl)-1-methyl-1H-pyrazol-5-yl)-2H-benzo[b][1,4]oxazin-3(4H)-one

According to the method of Example 73, 6-(4-bromo-1-methyl-1H-pyrazol-5-yl)-2H-benzo[b][1,4]oxazin-3(4H)-one (200 mg, 0.65 mmol) and 4-fluorophenylboronic acid (272 mg, 1.95 mmol) were reacted to give the title compound as a light gray solid (22 mg, 11%).

¹H-NMR (400 MHz, CDCl₃) δ: 8.21 (brs, 1H), 7.67 (s, 1H), 7.34 (m, 1H), 7.12 (m, 2H), 7.07 (d, J=8.2 Hz, 1H), 6.94 (m, 2H), 6.70 (d, J=2.0 Hz 1H), 4.69 (s, 2H), 3.78 (s, 3H); LCMS (ESI⁺) M+H⁺: 324.

### Example 301

### 6-(3-Ethyl-4-(4-fluorophenyl)-1-methyl-1H-pyrazol-5-yl)-2H-benzo[b][1,4]oxazin-3(4H)-one

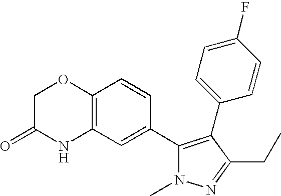

### 1-(3-Oxo-3,4-dihydro-2H-benzo[b][1,4]oxazin-6-yl)pentane-1,3-dione

According to the method of Example 71, 6-acetyl-2H-benzo[b][1,4]oxazin-3(4H)-one (5.0 g, 26.2 mmol) and ethyl propionate (10.7 g, 105 mmol) were reacted to give the title compound as an off-white solid (5.2 g, 79%).

LCMS (ESI⁻) M−H⁻: 246.

### 6-(3-Ethyl-1-methyl-1H-pyrazol-5-yl)-2H-benzo[b][1,4]oxazin-3(4H)-one

According to the method of Example 71, 1-(3-oxo-3,4-dihydro-2H-benzo[b][1,4]oxazin-6-yl)pentane-1,3-dione (1.0 g, 4.05 mmol) and 1-methylhydrazine (224 mL, 4.25 mmol) were reacted to give the title compound as a white solid 527 mg, 51%).

LCMS (ESI⁺) M+H⁺: 258.

### 6-(4-Bromo-3-ethyl-1-methyl-1H-pyrazol-5-yl)-2H-benzo[b][1,4]oxazin-3(4H)-one

According to the method of Example 56, 6-(3-ethyl-1-methyl-1H-pyrazol-5-yl)-2H-benzo[b][1,4]oxazin-3(4H)-one (527 mg, 2.05 mmol) and NBS (365 mg, 2.05 mmol) were reacted to give the title compound as a white solid 650 mg, 94%).

LCMS (ESI⁺) M+H⁺: 336, 338.

### 6-(3-Ethyl-4-(4-fluorophenyl)-1-methyl-1H-pyrazol-5-yl)-2H-benzo[b][1,4]oxazin-3(4H)-one

According to the method of Example 73, 6-(4-bromo-3-ethyl-1-methyl-1H-pyrazol-5-yl)-2H-benzo[b][1,4]oxazin-3(4H)-one (100 mg, 0.30 mmol) and 4-fluorophenylboronic acid (125 mg, 0.89 mmol) were reacted to give the title compound as a white solid (66 mg, 63%).

¹H-NMR (400 MHz, CDCl₃) δ: 7.92 (brs, 1H), 7.04 (m, 2H), 6.96 (m, 3H), 6.84 (dd, J=8.2, 2.0 Hz, 1H), 6.57 (d, J=2.0 Hz, 1H), 4.64 (s, 2H), 3.77 (s, 3H), 2.66 (q, J=7.6 Hz, 2H), 1.20 (t, J=7.6 Hz, 3H); LCMS (ESI⁺) M+H⁺: 352.

### Example 302

### 6-(4-(4-Fluoro-2-methylphenyl)-1-methyl-1H-pyrazol-5-yl)-2H-benzo[b][1,4]oxazin-3(4H)-one

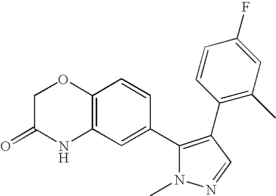

According to the method of Example 73, 6-(4-bromo-1-methyl-1H-pyrazol-5-yl)-2H-benzo[b][1,4]oxazin-3(4H)-one (227 mg, 0.74 mmol) and 4-fluoro-2-methylphenylboronic acid (340 mg, 2.21 mmol) were reacted to give the title compound as a white solid (87 mg, 35%).

¹H-NMR (400 MHz, CDCl₃) δ: 8.42 (brs, 1H), 7.48 (s, 1H), 6.98 (m, 2H), 6.81 (m, 3H), 6.56 (d, J=2.0 Hz, 1H), 4.63 (s, 2H), 3.87 (s, 3H), 2.05 (s, 3H); LCMS (ESI⁺) M+H⁺: 338.

### Example 303

### 6-(4-(4-Fluoro-2-methoxyphenyl)-1-methyl-1H-pyrazol-5-yl)-2H-benzo[b][1,4]oxazin-3(4H)-one

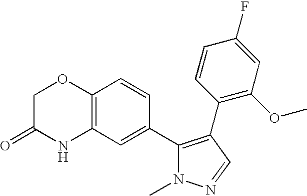

According to the method of Example 73, 6-(4-bromo-1-methyl-1H-pyrazol-5-yl)-2H-benzo[b][1,4]oxazin-3(4H)-one (150 mg, 0.49 mmol) and 4-fluoro-2-methoxyphenylboronic acid (248.2 mg, 1.46 mmol) were reacted to give the title compound as an off-white solid (108 mg, 63%).

¹H-NMR (400 MHz, CDCl₃) δ: 8.19 (brs, 1H), 7.67 (s, 1H), 7.00 (d, J=8.2 Hz, 1H), 6.89 (m, 2H), 6.64 (d, J=2.0 Hz, 1H), 6.58 (m, 1H), 6.52 (m, 1H), 4.65 (s, 2H), 3.81 (s, 3H), 3.65 (s, 3H); LCMS (ESI⁺) M+H⁺: 354.

### Example 304

### 8-Fluoro-6-(4-(4-fluorophenyl)-1,3-dimethyl-1H-pyrazol-5-yl)-2H-benzo[b][1,4]oxazin-3(4H)-one

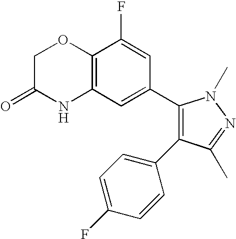

### 4-(Dimethylamino)-3-(4-fluorophenyl)but-3-en-2-one

According to the method of Example 55, 1-(4-fluorophenyl)propan-2-one (8.78 mL, 65.72 mmol) and N,N-dimethylformamide dimethyl acetal (14.02 mL, 105.1 mmol) were reacted to give the title compound as an off-white solid (4.4 g, 64%).

LCMS (ESI⁺) M+H⁺: 208.

### 4-(4-Fluorophenyl)-1,3-dimethyl-1H-pyrazole

To a stirred solution of 4-(dimethylamino)-3-(4-fluorophenyl)but-3-en-2-one (4.4 g, 21.2 mmol) in CH₃CN (100 mL) was added 1-methylhydrazine (1.34 mL, 25.5 mmol) and the mixture was heated at 40° C. for 20 hr. The reaction mixture was concentrated in vacuo. Flash chromatography of the residue on silica gel (50% EtOAc in petroleum ether) gave the title compound as a solid (3.3 g, 83%).

LCMS (ESI⁺) M+H⁺: 191.

### 5-Bromo-4-(4-fluorophenyl)-1,3-dimethyl-1H-pyrazole

To a stirred mixture of 4-(4-fluorophenyl)-1,3-dimethyl-1H-pyrazole (3.33 g, 17.5 mmol) in CH₃CN (300 mL) at room temperature was added NBS (6.23 g, 35.0 mmol) and stirring was continued for 16 hr. The reaction mixture was concentrated in vacuo and the residue was dissolved with water and DMF, extracted with EtOAc, and the organic layer was washed with water, 10% aqueous LiCl solution and brine, dried (MgSO₄) and concentrated in vacuo to give the title compound as a solid (4.4 g, 94%).

LCMS (ESI⁺) M+H⁺: 269, 271.

### 8-Fluoro-6-(4,4,5,5-tetramethyl-1,3,2-dioxaborolan-2-yl)-2H-benzo[b][1,4]oxazin-3(4H)-one

A mixture of 6-bromo-8-fluoro-2H-benzo[b][1,4]oxazin-3(4H)-one (200 mg, 0.81 mmol, Example 107), bis(pinacolato)diboron (413 mg, 1.63 mmol) and KOAc (239 mg, 2.44 mmol) in dioxane (12 mL) was degassed with N₂. PdCl₂(dppf)-DCM (80.3 mg, 0.098 mmol) was added and the mixture was heated at 90° C. for 16 hr. The mixture was cooled, partitioned between water and EtOAc, and filtered through Celite. The layers were separated and the EtOAc layer was washed with brine, dried (MgSO₄), and concentrated in vacuo. Flash chromatography of the residue on silica gel (EtOAc) gave the title compound as a white solid (238 mg, 99%).

LCMS (ESI⁻) M−H: 292.

### 8-Fluoro-6-(4-(4-fluorophenyl)-1,3-dimethyl-1H-pyrazol-5-yl)-2H-benzo[b][1,4]oxazin-3(4H)-one

A mixture of 8-fluoro-6-(4,4,5,5-tetramethyl-1,3,2-dioxaborolan-2-yl)-2H-benzo[b][1,4]oxazin-3(4H)-one (238 mg, 0.81 mmol), 5-bromo-4-(4-fluorophenyl)-1,3-dimethyl-1H-pyrazole (171 mg, 0.64 mmol) and KOAc (239 mg, 2.44 mmol) in dioxane (10 mL) was degassed with N₂. PdCl₂(dppf)-DCM (80.2 mg, 0.097 mmol) was added and the mixture was heated at 90° C. for 16 hr. The mixture was filtered through Celite (EtOAc), and the filtrate was washed with water and brine, dried (MgSO₄), and concentrated in vacuo. RP-HPLC of the residue gave the title compound as a white solid (2.2 mg, 0.76%).

¹H-NMR (400 MHz, CDCl₃) δ: 7.41 (brs, 1H), 7.00 (m, 4H), 6.71 (dd, J=10.2, 1.6 Hz, 1H), 6.34 (m, 1H), 4.72 (s, 2H), 3.77 (s, 3H), 2.29 (s, 3H); LCMS (ESI⁺) M+H⁺: 356.

### Example 305

### 6-(4-(4-Fluorophenyl)-1,3-dimethyl-1H-pyrazol-5-yl)-2H-pyrido[3,2-b][1,4]oxazin-3(4H)-one

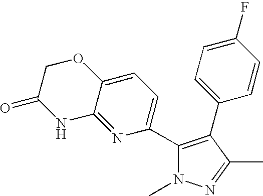

### 4-(4-Fluorophenyl)-1,3-dimethyl-1H-pyrazol-5-ylboronic acid

To a stirred Solution of 5-bromo-4-(4-fluorophenyl)-1,3-dimethyl-1H-pyrazole (100 mg, 0.37 mmol) in THF (2 mL) was added triisopropyl borate (0.51 mL, 2.23 mmol) and the mixture was cooled to −78° C. N-butyllithium (0.35 mL, 1.6 M in hexanes, 0.56 mmol) was added dropwise, and stirring was continued for 30 min at −78° C. The reaction mixture was brought to 0° C. for 1 hr, and then quenched with saturated aqueous NH₄Cl solution and stirred at room temperature for 1 hr. The mixture was extracted with EtOAc, and the organic layer was washed with brine, dried (MgSO₄) and concentrated in vacuo to give the title compound as an amber oil (80 mg, 92%, 1:1 mixture with 4-(4-fluorophenyl)-1,3-dimethyl-1H-pyrazole).

LCMS (ESI⁺) M+H⁺: 235.

### 6-(4-(4-Fluorophenyl)-1,3-dimethyl-1H-pyrazol-5-yl)-2H-pyrido[3,2-b][1,4]oxazin-3(4H)-one

According to the method of Example 34, 4-(4-fluorophenyl)-1,3-dimethyl-1H-pyrazol-5-ylboronic acid (80 mg, 0.34 mmol) and 6-bromo-2H-pyrido[3,2-b][1,4]oxazin-3(4H)-one (78.3 mg, 0.34 mmol) were reacted to give the title compound as a white solid (21 mg, 18%).

¹H-NMR (400 MHz, CDCl₃) δ: 8.06 (brs, 1H), 7.10 (m, 3H), 7.00 (m, 2H), 6.64 (d, J=8.2 Hz, 1H), 4.72 (s, 2H), 3.93 (s, 3H), 2.26 (s, 3H); LCMS (ESI⁺) M+H⁺: 339.

### Example 306

### 8-Chloro-6-(4-(4-fluorophenyl)-1,3-dimethyl-1H-pyrazol-5-yl)-2H-benzo[b][1,4]oxazin-3(4H)-one

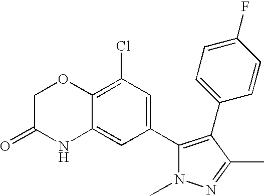

According to the method of Example 34, 4-(4-fluorophenyl)-1,3-dimethyl-1H-pyrazol-5-ylboronic acid (245 mg, 1.05 mmol) and 6-bromo-8-chloro-2H-benzo[b][1,4]oxazin-3(4H)-one (102 mg, 0.39 mmol, Example 109) were reacted to give the title compound as an off white solid (38 mg, 10%).

¹H-NMR (400 MHz, CDCl₃) δ: 8.27 (brs, 1H), 7.04 (m, 2H), 6.98 (m, 3H), 6.48 (d, J=2.0 Hz, 1H), 4.74 (s, 2H), 3.75 (s, 3H), 2.27 (s, 3H); LCMS (ESI⁺) M+H⁺: 372.

### Example 307

### 3-(4-Fluorophenyl)-4-(3-oxo-3,4-dihydro-2H-benzo[b][1,4]oxazin-6-yl)-1-(2,2,2-trifluoroethyl)-1H-pyrrole-2,5-dione

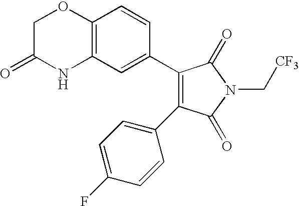

### 3-(4-Fluorophenyl)-4-(3-oxo-3,4-dihydro-2H-benzo[b][1,4]oxazin-6-yl)furan-2,5-dione

To a solution of 6-(4-(4-fluorophenyl)-5-oxo-2,5-dihydrofuran-3-yl)-2H-benzo[b][1,4]oxazin-3(4H)-one (6.40 g, 19.7 mmol, Example 61) in acetonitrile (100 mL) was added DBU (8.99 g, 59.0 mmol) and O₂ (gas) was bubbled through the solution for 5 hr at 40° C. The reaction mixture was diluted with EtOAc and washed with 6N HCl and brine, dried (MgSO₄) and concentrated in vacuo to give the title compound (5.99 g, 90%).

¹H-NMR (400 MHz, CDCl₃) δ: 9.06 (s, 1H), 7.60 (dd, J=8.6, 5.5 Hz, 2H), 7.28 (m, 3H), 7.12 (dd, J=8.4, 2.0 Hz, 1H), 6.97 (d, J=8.4 Hz, 1H), 4.65 (s, 2H), 4.38 (q, J=9.2 Hz, 2H).

### 3-(4-Fluorophenyl)-4-(3-oxo-3,4-dihydro-2H-benzo[b][1,4]oxazin-6-yl)-1-(2,2,2-trifluoroethyl)-1H-pyrrole-2,5-dione

A stirred solution of 3-(4-fluorophenyl)-4-(3-oxo-3,4-dihydro-2H-benzo[b][1,4]oxazin-6-yl)furan-2,5-dione (237 mg, 0.70 mmol) and 2,2,2-trifluoroethanamine (415 mg, 4.19 mmol) in DMF (2.5 mL) was heated at 90° C. overnight. The reaction mixture was diluted with EtOAc, washed with water and brine, dried (MgSO₄) and concentrated in vacuo to give the title compound as a dark red solid (40.0 mg, 14%).

¹H-NMR (400 MHz, acetone-d₆) δ: 9.72 (s, 1H), 7.58 (dd, J=8.6, 5.5 Hz, 1H), 7.22 (d, J=10.1 Hz, 2H), 7.12 (dd, J=8.2, 2.0 Hz, 1H), 6.98 (d, J=8.2 Hz, 1H), 4.65 (s, 2H), 4.41 (q, J=9.3 Hz, 2H); LCMS (ESI⁻) M−H⁻: 419.

### Example 308

### 6-(3-(4-Fluoro-2-methylphenyl)-1-methyl-1H-pyrazol-4-yl)-2H-benzo[b][1,4]oxazin-3(4H)-one

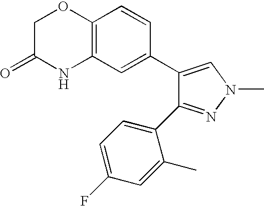

### 2-(3-Oxo-3,4-dihydro-2H-benzo[b][1,4]oxazin-6-yl)acetyl chloride

To a stirred solution of 2-(3-oxo-3,4-dihydro-2H-benzo[b][1,4]oxazin-6-yl)acetic acid (5.05 g, 24.37 mmol) in THF (122 mL) at room temperature was slowly added oxalyl chloride (2.339 mL, 26.81 mmol) and then DMF (3 drops) was added, and stirring was continued for 6 hr. The reaction mixture was concentrated in vacuo and azeotroped with toluene to give the title compound as a brown oil (5.50 g, 99%).

LCMS (ESI⁻) M−H⁻: 224.

### N-Methoxy-N-methyl-2-(3-oxo-3,4-dihydro-2H-benzo[b][1,4]oxazin-6-yl)acetamide

To a stirred solution of 2-(3-oxo-3,4-dihydro-2H-benzo[b][1,4]oxazin-6-yl)acetyl chloride (5.50 g, 24.38 mmol) in DCM (122 mL) was added N,O-dimethylhydroxylamine HCl (3.57 g, 36.56 mmol). Triethylamine (17.0 mL, 121.9 mmol) was added slowly and the resulting solution was stirred overnight. The reaction mixture was poured into water, extracted three times with EtOAc, washed with brine, dried (MgSO₄), and concentrated in vacuo to give the title compound (2.10 g, 34%).

LCMS (ESI⁻) M−H⁻: 249.

### 6-(2-(4-Fluoro-2-methylphenyl)-2-oxoethyl)-2H-benzo[b][1,4]oxazin-3(4H)-one

To a solution of (4-fluoro-2-methylphenyl)magnesium bromide, (4.39 g, 20.58 mmol) in THF (100 mL) at −78° C. was added portion-wise N-methoxy-N-methyl-2-(3-oxo-3,4-dihydro-2H-benzo[b][1,4]oxazin-6-yl)acetamide (2.06 g, 8.23 mmol). The reaction mixture was warmed to room temperature and stirred overnight. The mixture was diluted with water, extracted three times with EtOAc, and the organic layer was dried (MgSO₄) and concentrated in vacuo to give the title compound as a white solid (559 mg, 23%).

LCMS (ESI⁻) M−H⁻: 298.

### 6-(3-(Dimethylamino)-1-(4-fluoro-2-methylphenyl)-1-oxoprop-2-en-2-yl)-2H-benzo[b][1,4]oxazin-3(4H)-one

A mixture of N,N-dimethylformamide dimethyl acetal (8.67 mL, 0.65 mmol) and 6-(2-(4-fluoro-2-methylphenyl)-2-oxoethyl)-2H-benzo[b][1,4]oxazin-3(4H)-one (195 mg, 0.65 mmol) was heated to 80° C. and stirred overnight. Concentration in vacuo gave the title compound (230 mg, 99%).

LCMS (ESI⁻) M−H⁻: 353.

### 6-(3-(4-Fluoro-2-methylphenyl)-1-methyl-1H-pyrazol-4-yl)-2H-benzo[b][1,4]oxazin-3(4H)-one

To a stirred solution of methylhydrazine (0.38 mL, 7.15 mmol) in IPA (35 mL) was added TFA (55 μL, 0.72 mmol) and then 6-(3-(dimethylamino)-1-(4-fluoro-2-methylphenyl)-1-oxoprop-2-en-2-yl)-2H-benzo[b][1,4]oxazin-3(4H)-one (230 mg, 0.65 mmol) was added, and the solution was heated at 65° C. for 3 days. Water was added and the mixture was extracted three times with EtOAc, and the organic layer was dried (MgSO₄) and concentrated in vacuo. Flash chromatography of the residue on silica gel (EtOAc/hexanes) gave the title compound as a white solid (20 mg, 9%), as an 85:15 mixture of regioisomers.

¹H-NMR (400 MHz, DMSO-d₆) δ: 10.66 (brs, 1H), 7.95 (s, 1H), 7.18 (dd, J=8.6, 6.2 Hz, 1H), 7.10 (dd, J=10.2, 2.3 Hz, 1H), 7.02 (td, J=8.6, 2.7 Hz, 1H), 6.80 (d, J=8.6 Hz, 1H), 6.64 (m, 1H), 6.60 (dd, J=8.2, 2.0 Hz, 1H), 4.51 (s, 2H), 3.89 (s, 3H), 2.00 (s, 3H); LCMS (ESI⁻) M−H⁻: 336.

### Example 309

### Cis-6-(5-oxo-4-phenyltetrahydrofuran-3-yl)-2H-benzo[b][1,4]oxazin-3(4H)-one

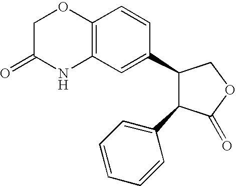

A mixture of 6-(5-oxo-4-phenyl-2,5-dihydrofuran-3-yl)-2H-benzo[b][1,4]oxazin-3(4H)-one (500 mg, 1.63 mmol) and 10% Pd/C (866 mg) in ethyl acetate (5 mL) and ethanol (20 mL) was agitated in a Parr hydrogenator under H₂ (60 psi). The reaction mixture was filtered through glass fiber filter and the filtrate was dried (Na₂SO₄) and concentrated in vacuo. Flash chromatography of the residue on silica gel chromatography (20%-50% EtOAc in hexanes) gave the title compound as a white solid: (50.0 mg, 10%).

¹H-NMR (400 MHz, DMSO-d₆) δ: 9.59 (brs, 1H), 7.12 (m, 2H), 6.96 (m, 3H), 6.68 (d, J=8.2 Hz, 1H), 6.59 (d, J=2.0 Hz, 1H), 6.48 (dd, J=8.2, 2.0 Hz, 1H), 4.78 (dd, J=9.3, 6.2 Hz, 1H), 4.58 (dd, J=9.3, 3.1 Hz, 1H), 4.47 (d, J=8.6 Hz, 1H), 4.46 (s, 2H), 4.11 (ddd, J=9.3, 6.2, 3.1 Hz, 1H); LCMS (ESI⁻) M−H⁻: 308.

### Example 310

### 6-(3-(1,1-Difluoroethyl)-1-(4-fluoro-2-methylphenyl)-1H-pyrazol-5-yl)-8-methyl-2H-benzo[b][1,4]oxazin-3(4H)-one

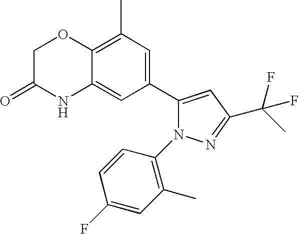

### 4,4-Difluoro-1-(8-methyl-3-oxo-3,4-dihydro-2H-benzo[b][1,4]oxazin-6-yl)pentane-1,3-dione

To a stirred mixture of NaH (2.96 mg, 7.41 mmol) in THF (3 mL) was carefully added ethyl 2,2-difluoropropanoate (1.02 g, 7.41 mmol). To this resulting mixture were added sequentially 6-acetyl-8-methyl-2H-benzo[b][1,4]oxazin-3(4H)-one (380 mg, 1.85 mmol), ethanol (2 drops) and a solution of 2,3,11,12-dibenzo-1,4,7,10,13,16-hexaoxacyclooctadeca-2,11-diene (11 mg, 0.03 mmol) in THF (2 mL). The mixture was refluxed for overnight, cooled, and partitioned between 10% H₂SO₄ and EtOAc. The organic layer was separated and washed with water, 5% aqueous Na₂CO₃, water and brine, dried (Mg₂SO₄), and concentrated in vacuo. The residue was triturated with ether and filtered to give the title compound (172 mg, 31%).

¹H-NMR (400 MHz, CDCl₃) δ: 8.82 (brs, 1H), 7.52 (dd, J=8.4, 2.0 Hz, 1H), 7.42 (d, J=2.0 Hz, 1H), 7.12 (d, J=8.4 Hz, 1H), 6.10 (s, 1H), 4.71 (s, 2H), 2.19 (s, 3H); LCMS (APCI⁻): M−H⁻: 296.

### 6-(3-(1,1-Difluoroethyl)-1-(4-fluoro-2-methylphenyl)-1H-pyrazol-5-yl)-8-methyl-2H-benzo[b][1,4]oxazin-3(4H)-one

A mixture of 1-(4-fluoro-2-methylphenyl)hydrazine hydrochloride (107 mg, 0.61 mmol) and triethylamine (112 μL, 0.81 mmol) in IPA (3.0 mL), was stirred at room temperature for 15 min. To the mixture was added TFA (95 μL, 1.24 mmol) and stirring was continued for 15 min. 4,4-Difluoro-1-(8-methyl-3-oxo-3,4-dihydro-2H-benzo[b][1,4]oxazin-6-yl)pentane-1,3-dione (172 mg, 0.58 mmol) was added and the reaction mixture was heated to 60° C. for 5 hr. Most of the IPA was removed in vacuo, water was added, and the pH adjusted to 5-6 with 1M NaOH. The resultant solids were collected by filtration and washed with petroleum ether. Flash chromatography on silica gel (2% MeOH in DCM) gave the title compound as a beige solid (216 mg, 73%).

¹H-NMR (400 MHz, CDCl₃) δ: 8.07 (s, 1H), 7.24 (m, 1H), 6.96 (m, 2H), 6.69 (m, 1H), 6.67 (s, 1H), 6.34 (d, J=1.6, 1H), 4.62 (s, 2H), 2.14 (s, 3H), 2.07 (t, J=18.4 Hz, 3H), 1.96 (s, 3H); LCMS (ESI⁻), M−H⁻: 400.

### Preparation 133

### Preparation of 4,4,4-trifluoro-1-(8-methyl-3-oxo-3,4-dihydro-2H-benzo[b][1,4]oxazin-6-yl)butane-1,3-dione

### 4-Hydroxy-3-methyl-5-nitroacetophenone

To a solution of 4-hydroxy-3-methylacetophenone (100 g, 666 mmol) in acetic acid (444 mL) was added nitric acid (70%, 31.0 ml, 732 mmol) at room temperature. The resulting solution was stirred at room temperature for 24 hr. The reaction mixture was poured into water and the white solid precipitate was collected by vacuum filtration to afford the title compound (77.0 g, 59%).

¹H-NMR (400 MHz, acetone-d₆) δ: 8.57 (d, J=2.3 Hz, 1H), 8.18 (m, 1H), 2.62 (s, 3H), 2.38 (s, 3H).

### Methyl 2-(4-acetyl-2-methyl-6-nitrophenoxy)acetate

A mixture of 4-hydroxy-3-methyl-5-nitroacetophenone (77.0 g, 395 mmol), methyl 2-bromoacetate (90.5 g, 592 mmol), K₂CO₃ (164 g, 1.18 mol) and DMF (800 mL) was stirred at room temperature overnight. The reaction mixture was poured into water to precipitate a white solid that was collected by vacuum filtration to afford the title compound (99.0 g, 94%).

¹H-NMR (400 MHz, CD₃OD) δ: 8.26 (d, J=2.3 Hz, 1H), 8.14 (m, 1H), 4.85 (s, 2H), 3.79 (s, 3H), 2.61 (s, 3H), 2.46 (s, 3H).

### 6-Acetyl-8-methyl-2H-benzo[b][1,4]oxazin-3(4H)-one

To a solution of methyl 2-(4-acetyl-2-methyl-6-nitrophenoxy)acetate (99.0 g, 370 mmol) in acetic acid (750 mL) was slowly added Zn dust (115.08 g, 1759.9 mmol) to avoid an excessively exothermic reaction. Upon completion of the addition, the reaction mixture was heated at 100° C. for 45 min, at which point the hot reaction mixture was filtered hot through a Buchner funnel equipped with a paper filter. The filter cake was added to DMF and this mixture was heated to 80° C. and stirred at this temperature for 30 min. The hot mixture was filtered through a paper filter. The filtrates were poured into water and the white precipitate was collected to afford the title compound (72.0 g, 95%).

LCMS (ESI⁻), M−H⁺: 204.

### 4,4,4-Trifluoro-1-(8-methyl-3-oxo-3,4-dihydro-2H-benzo[b][1,4]oxazin-6-yl)butane-1,3-dione

To a slurry of 60% NaH (56.1 g, 1.40 mol) in THF (4.6 L) was slowly added ethyl 2,2,2-trifluoroacetate (167.4 ml, 1.41 mol). To this mixture was added 6-acetyl-8-methyl-2H-benzo[b][1,4]oxazin-3(4H)-one (72.0 g, 351 mmol) as a solid, and then dibenzo-18-crown-6 (0.97 g, 2.69 mmol) and 1.00 ml of ethanol (absolute) were added. The resulting mixture was heated at 65° C. for 2 hr, poured into 1N-HCl and extracted with ethyl acetate. The organic layer was washed with water, dried and concentrated. The residue was triturated with ether/petroleum ether to give the title compound as an off-white solid (37.20 g, 35%).

LCMS (ESI⁻), M−H⁺: 300.

### Preparation 134

### 4,4,4-Trifluoro-1-(3-oxo-3,4-dihydro-2H-benzo[b][1,4]oxazin-6-yl)butane-1,3-dione

To a mixture of NaH (2.51 g, 105 mmol) in THF (100 mL) was carefully added ethyl 2,2,2-trifluoroacetate (12.5 mL, 105 mmol), observing both effervescence and a slight exotherm. To this resulting mixture were added sequentially 6-acetyl-2H-benzo[b][1,4]oxazin-3(4H)-one (5.00 g, 26.2 mmol), ethanol (2.50 mL) and a solution of [2,4]-dibenzo-18-crown-6 (150 mg, 0.418 mmol) in THF (50.0 mL). The mixture was refluxed for 16 hr, cooled, and partitioned between 10% H₂SO₄ (200 mL) and EtOAc (200 mL). The organic layer was separated and washed with water (200 mL), saturated aqueous NaHCO₃ (200 mL), water (200 mL) and brine (200 mL), dried (Na₂SO₄) and concentrated in vacuo. The residue was triturated with ether to give the title compound as a yellow solid (6.67 g, 80%).

¹H-NMR (400 MHz, DMSO-d₆) δ: 10.88 (s, 1H), 7.63 (dd, J=8.5, 2.1 Hz, 1H), 7.49 (d, J=2.1 Hz, 1H), 7.04 (d, J=8.4 Hz, 1H), 6.30 (s, 1H), 4.69 (s, 2H) and 10.81 (s, 1H), 7.58 (dd, J=8.4, 1.6 Hz, 1H), 7.48 (d, J=1.9 Hz, 1H), 7.11 (s, 1H), 7.00 (d, J=8.4 Hz, 1H), 4.67 (s, 2H), consistent with a mixture of enolic tautomers; LCMS (ESI⁻), M−H⁻: 286.

### Preparation 135

### 1-(4-Fluoro-2-methylphenyl)hydrazine hydrochloride

To a solution of 4-fluoro-2-methylaniline (125 g, 1.00 mol) in c-HCl (1000 mL) was added NaNO₂ (137 g, 2.00 mol) as a solid with cooling and the mixture was stirred at 0° C. for 2 hr. While at 0° C., to the mixture was added SnCl₂ (474 g, 2.50 mol) as a solid. The reaction mixture was stirred at 0° C. for 3 hr and room temperature overnight, and poured into a separatory funnel and washed with ether (250 mL). The aqueous layer was slowly and carefully added to aqueous NaOH under ice cooling to basify the solution. The basic aqueous layer was extracted with ethyl acetate, and the organic layer was dried and concentrated to give 1-(4-fluoro-2-methylphenyl)hydrazine, that solidified upon standing. The residue was dissolved with a minimal amount of ether and precipitated with 4N HCl/dioxane to afford the title compound as a white solid (85.0 g, 48%). The material was used in subsequent reactions without further purification.

LCMS (ESI⁺), M+H⁺: 141.

### Example 311

### Methyl 3-[5-(3-oxo-3,4-dihydro-2H-1,4-benzoxazin-6-yl)-6-phenyl-6H-1,3-thiazin-2-yl]propanoate

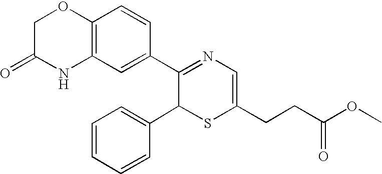

A mixture of 2-(3-oxo-3,4-dihydro-2H-1,4-benzoxazin-6-yl)-3-phenylacrylaldehyde (0.3 g), methyl 4-amino-4-thioxobutanoate (0.18 g), 4N-hydrochloric acid in dioxane (3 mL) was stirred at room temperature for 12 hr. Methanol (3 mL) was added to the mixture, and the mixture was refluxed for 4 hr. The mixture was concentrated in vacuo, and then saturated aqueous sodium bicarbonate solution and water were added to the residue. The mixture was extracted with ethyl acetate, and the organic layer was washed with water and brine, dried over Na₂SO₄ and concentrated in vacuo. The residue was purified by chromatography on silica gel (hexane→hexane:ethyl acetate=2:3) and followed by crystallization from ethyl acetate/hexane to give the title compound as crystals (0.17 g).

mp. 119-121° C.

¹H-NMR (300 MHz, DMSO-d₆) δ: 2.41-2.80 (m, 4H), 3.54 (s, 3H), 4.54 (s, 2H), 5.37 (s, 1H), 6.90 (d, J=8.4 Hz, 1H), 6.98 (d, J=2.1 Hz, 1H), 7.07 (dd, J=8.4, 2.1 Hz, 1H), 7.15-7.35 (m, 5H), 7.47 (s, 1H), 10.70 (s, 1H).

### Experimental Example 1

The following procedures described in this Example were carried out according to the methods described in Molecular Cloning—Cold Spring Harbor Laboratory (1989) or protocols specified by manufacturers.

(1) Cloning of Human Mineralocorticoid Receptor (hMR) cDNA

hMR cDNA was amplified by polymerase chain reaction (PCR) from human kidney cDNA library. Full-length cDNA was constructed from two fragments of hMR cDNA amplified separately. The primers were designed referring to the nucleotide sequence of hMR cDNA reported by Arriza et. al (Science 1987; 237: 268-275).

(SEQ ID No. 1)

hMR-U: 5′-GGGGCTCGAGGCAGGGATGGAGACCAAAGGCTAC-3′

(SEQ ID No. 2)

hMR-1911L: 5′-GGATACCCATCACTTCTTCTAGACGACAGG-3′

(SEQ ID No. 3)

hMR-1686U: 5′-AGTGGGTATTAAACAAGAACCAGATGACGG-3′

(SEQ ID No. 4)

hMR-L: 5′-GGGAGGTACCTTCTGGGCAGCGGGCAGTCACTTC-3′

The PCR reactions were carried out using Pyrobest® DNA polymerase (Takara). The PCR products were electrophoresed in agarose gel, and 1.7 kb (region (i)) and 1.5 kb (region (ii)) DNA fragments were recovered. Each DNA fragment was inserted into pCR®4Blunt-TOPO® vector (Invitrogen). The resulting plasmids thus obtained were designated as pB-hMR (i) and pB-hMR (ii). To obtain the full-length hMR cDNA, pB-hMR (i) was digested with XhoI and PvuI, and pB-hMR (ii) was digested with PvuI and KpnI, respectively, and the two cDNA fragments were ligated into pBlueScript®IISK+ vector (Stratagene). The resulting plasmid thus obtained was designated as pB-hMR.

### (2) Construction of hMR Expression Plasmid

pMCMVneo (described in WO03/099793) was digested with XhoI and KpnI, and 5.6 kb fragment was ligated with 2.9 kb hMR cDNA fragment obtained by digestion of pB-hMR (described in above (1)) with XhoI and KpnI. The plasmid thus obtained was designated as pMCMVneo-hMR.

### (3) Expression of hMR in FreeStyle 293 Cells and Preparation of Cell Lysate

FreeStyle 293 cells were inoculated at 1×10⁸ cells in 93 ml FreeStyle™ 293 Expression Medium (Invitrogen) in a 500 ml Erlenmeyer flask and cultured at 37° C. under 8% CO₂ for 1 hr. The cells were treated with 6.7 ml of the transfection mixture containing 100 μg of pMCMVneo-hMR obtained in above (2) and 133 μl of FreeStyle™ 293 Transfection Reagent (Invitrogen). The transfected cells were cultivated for 48 hr at 37° C. in 8% CO₂ atmosphere. The cultivated cells were centrifuged and washed with TEG buffer (10 mM Tris-HCl (pH 7.2), 50 mM EDTA, 10% glycerol), and resuspended in 10 ml TEGM buffer (10 mM Tris-HCl (pH 7.2), 1 mM EDTA, 10% glycerol, 1 mM β-mercaptoethanol, 10 mM sodium molybdate, 1 mM dithiothreitol, 2 tablets/100 ml of protease inhibitor cocktail tablets (Roche)). The cell suspension was frozen with liquid nitrogen and thawed on ice, and ultra-centrifuged at 225,000×g for 20 min at 4° C. The supernatant fraction including hMR (hMR lysate) was collected and stored at −80° C.

### (4) Measurement of Inhibition Activity Against Binding of hMR and Aldosterone

[³H]-Aldosterone (Amersham Biosciences) as ligand was added at 10 nM to the reaction mixture including test compound at various concentration and hMR lysate (1.0 mg/ml) obtained in above (3) and mixture was filled up to 50.5 μl with TEGM buffer. The reaction mixture was incubated for 16 hr at 4° C. and 35 μl of dextran/gelatin coated charcoal suspension (5% charcoal, 0.5% dextran T-70 (Amersham Biosciences), 0.1% gelatin (SIGMA), 10 mM Tris HCl (pH 7.2), 1 mM EDTA) was added thereto to separate bound and free radioactive aldosterone. The mixture containing charcoal was incubated for 10 min at 4° C. and centrifuged at 910×g for 10 min at 4° C. Radioactivity in 30 μl of the supernatant was measured by TopCount™ (Packard).

For the determination of nonspecific binding, cold Aldosterone instead of drug was added to reaction mixture at 100 μM. Specific binding was determined by subtracting nonspecific binding from total binding.

### (5) Experimental Results

Table 1 shows inhibition rate of compounds at 10⁻⁵ M. From the results of Table 1, it is clear that compound (I) and a salt thereof of the present invention have superior MR antagonistic activity.

TABLE 1

| Example Compound | Inhibition rate (at 10⁻⁵ M) |
|---|---|
| Example 3 | +++ |
| Example 5 | +++ |
| Example 7 | +++ |
| Example 14 | +++ |
| Example 18 | +++ |
| Example 19 | +++ |
| Example 20 | +++ |
| Example 24 | +++ |
| Example 26 | +++ |
| Example 31 | +++ |
| Example 34 | +++ |
| Example 35 | +++ |
| Example 43 | +++ |
| Example 44 | +++ |
| Example 49 | +++ |
| Example 53 | +++ |
| Example 57 | +++ |
| Example 59 | +++ |
| Example 65 | +++ |
| Example 70 | +++ |
| Example 71 | +++ |
| Example 74 | +++ |
| Example 82 | +++ |
| Example 86 | +++ |
| Example 93 | +++ |
| Example 96 | +++ |
| Example 100 | +++ |
| Example 107 | +++ |
| Example 108 | +++ |
| Example 110 | +++ |
| Example 118 | +++ |
| Example 130 | +++ |
| Example 158 | +++ |
| Example 185 | +++ |
| Example 195 | +++ |
| Example 255 | +++ |
| Example 288 | +++ |
| Example 310 | +++ |

+++ ≧ 70%,
70% ≧ ++ > 50%,
50% ≧ + > 30%,

The mineralocorticoid receptor antagonist of the present invention (e.g., hypertension therapeutic agent etc.) can be produced, for example, according to the following formulations.

In the following formulations, as the components (additive) other than the active ingredient, those recited in the Japan Pharmacopoeia, the Japan Pharmacopoeia Japanese Pharmaceutical Codex or Japanese Pharmaceutical Excipients and the like can be used.

### 1. Capsule

| | |
|---|---|
| (1) compound obtained in Example 1 | 40 mg |
| (2) lactose | 70 mg |
| (3) microcrystalline cellulose | 9 mg |
| (4) magnesium stearate | 1 mg |
| 1 capsule | 120 mg |

(1), (2), (3) and ½ of (4) are admixed and granulated. The remaining (4) is added and the whole is sealed in a gelatin capsule.

### 2. Tablet

| | |
|---|---|
| (1) compound obtained in Example 1 | 40 mg |
| (2) lactose | 58 mg |
| (3) cornstarch | 18 mg |
| (4) microcrystalline cellulose | 3.5 mg |
| (5) magnesium stearate | 0.5 mg |
| 1 tablet | 120 mg |

(1), (2), (3), ⅔ of (4) and ½ of (5) are admixed and granulated. The remaining (4) and (5) are added to the granules and the mixture is compression-molded into a tablet.

### 3. Capsule

| | |
|---|---|
| (1) compound obtained in Example 55 | 40 mg |
| (2) lactose | 70 mg |
| (3) microcrystalline cellulose | 9 mg |
| (4) magnesium stearate | 1 mg |
| 1 capsule | 120 mg |

(1), (2), (3) and ½ of (4) are admixed and granulated. The rest of (4) is added and the whole is sealed in a gelatin capsule.

### 4. Tablet

| | |
|---|---|
| (1) compound obtained in Example 55 | 40 mg |
| (2) lactose | 58 mg |
| (3) cornstarch | 18 mg |
| (4) microcrystalline cellulose | 3.5 mg |
| (5) magnesium stearate | 0.5 mg |
| 1 tablet | 120 mg |

(1), (2), (3), ⅔ of (4) and ½ of (5) are admixed and granulated. The remaining (4) and (5) are added to the granules and the mixture is compression-molded into a tablet.

### INDUSTRIAL APPLICABILITY

The compound of the present invention has a superior mineral corticoidreceptorantagonistic action and is useful as an agent for the prophylaxis or treatment of hypertension, cardiac failure and the like, a compound having a fused heterocycle, or a prodrug thereof, or a salt thereof; and an agent for the prophylaxis or treatment of hypertension, cardiac failure and the like.

1. A compound of the formula (Ia): 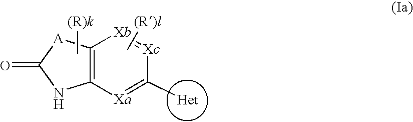
wherein
A is a group represented by the formula:
—X₁ [symbol] X₂ [symbol] X₃—
wherein
X₁ and X₂ are the same or different and each is a chemical bond, CH₂, CH, O, NH, N, S, SO or SO₂;
X₃ is CH₂, CH, O, NH, N, S, SO or SO₂; and [symbol]
is a single bond or a double bond;
provided that
when
—X₁ [symbol] X₂— is —X₁═X₂—,
then
—X₂ [symbol] X₃— should be —X₂—X₃—;
R and R′ are the same or different and each is an optionally substituted aliphatic hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group or an acyl group, or two R optionally form a spiro ring together with a carbon atom they are bonded to;
k is an integer of 0 to 4;
l is an integer of 0 to 3;
Xₐ is CH or N;
X_{b} is CH or N;
X_{c} is CH or N; and
a group represented by the formula: 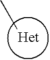
is a heterocyclic group represented by the formula: 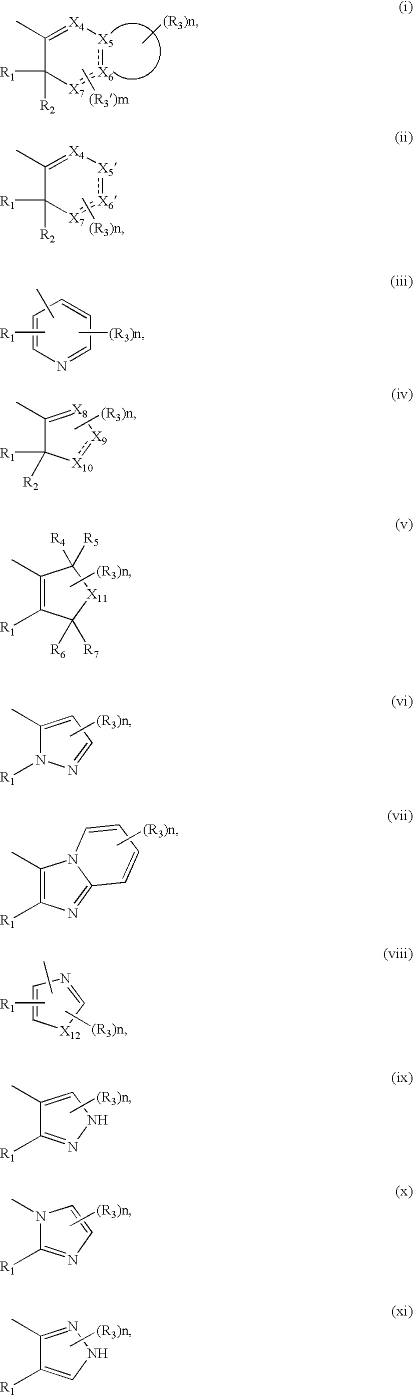 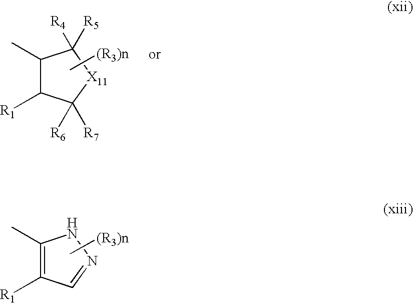
wherein
the formula: 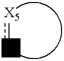
which partially constitutes the fused ring in the heterocyclic group represented by the formula (i), is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group, or
two R₃ optionally form, together with two adjacent atoms they are bonded to, a 3- to 7-membered ring which optionally contains, as a ring-constituting member, one or more members selected from O, N, S, SO and SO₂;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₆ and R₇ in combination optionally form an oxo group;
provided that at least one of a pair of R₄ and R₅ and a pair of R₆ and R₇ should form an oxo group;
m and n are the same or different and each is an integer of 0 to 4;
X₄ is CH or N;
X₅ and X₆ are the same or different and each is CH, C or N;
X₅′ and X₆′ are the same or different and each is CH₂, CH, NH, N, O, S, SO or SO₂;
X₇ is CH₂, CH, NH, N, O, S, SO or SO₂;
X₈ is CH or N;
X₉ is CH₂, CH, NH, N, O, S, SO or SO₂;
X₁₀ is CH₂, CH, NH, N, O, S, SO or SO₂;
X₁₁ is NH, O, S, SO or SO₂;
X₁₂ is O or S; and [symbol]
is a single bond or a double bond;
provided that
when
X₅ [symbol] X₆ is X₅═X₆,
then
X₆ [symbol] X₇ should be X₆—X₇, and
when
X₅′ [symbol] X₆′ is X₅′═X₆′,
then
X₆′ [symbol] X₇ should be X₆′—X₇;
with the proviso that
1) when the group represented by the formula: 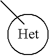
is a heterocyclic group represented by the formula: 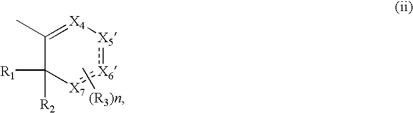
then at least one of R₁ and R₂ should be an optionally substituted aryl group or an optionally substituted heteroaryl group,
2) when the group represented by the formula: 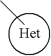
is a heterocyclic group represented by the formula: 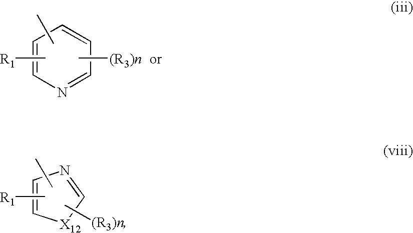
then the carbon atom to which the group represented by the formula: 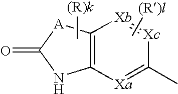
is bonded and the carbon atom to which R₁ is bonded should be adjacent to each other, and R₁ should be an optionally substituted aryl group or an optionally substituted heteroaryl group,
3) when the group represented by the formula:
—X₁ [symbol] X₂ [symbol] X₃— is —CH₂—O—, and the group represented by the formula: 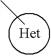
is a heterocyclic group represented by the formula: 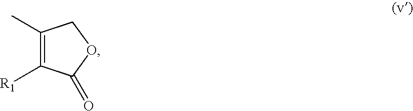
then R₁ should not be phenyl, 4-methoxyphenyl, 3,4-dimethoxyphenyl and 4-chlorophenyl,
4) when the group represented by the formula:
—X₁ [symbol] X₂ [symbol] X₃— is —CH₂—O—, and the group represented by the formula: 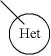
is a heterocyclic group represented by the formula: 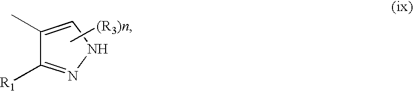
then R₁ should not be an optionally substituted 2-pyridyl,
5) when the group represented by the formula:
—X₁ [symbol] X₂ [symbol] X₃— is —CH₂—O—, and the group represented by the formula: 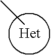
is a heterocyclic group represented by the formula: 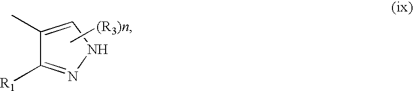
wherein R₁ is an optionally substituted phenyl, then —NH— group in the pyrazole ring as illustrated above should be substituted by R₃,
6) when the group represented by the formula:
—X₁ [symbol] X₂ [symbol] X₃— is —O—, —CH₂—O—, —CH₂—S— or —CH═CH—, and the group represented by the formula: 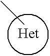
is a heterocyclic group represented by the formula: 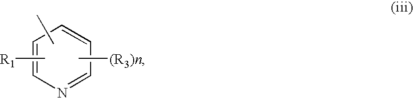
then R₁ should not be a halogen atom and trifluoromethyl,
7) when the group represented by the formula:
—X₁ [symbol] X₂ [symbol] X₃— is —NH— or —CH₂—NH—, and the group represented by the formula: 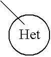
is a heterocyclic group represented by the formula: 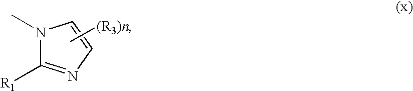
then R₁ should not be an alkyl group,
8) when the group represented by the formula:
—X₁ [symbol] X₂ [symbol] X₃— is —CH₂—O—, and the group represented by the formula: 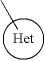
is a heterocyclic group represented by the formula: 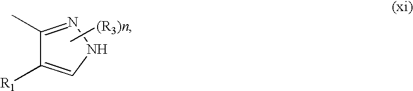
then R₁ should be an optionally substituted aryl group or an optionally substituted heteroaryl group,
9) when the group represented by the formula:
—X₁ [symbol] X₂ [symbol] X₃— is —S— or —CH₂—O—, and the group represented by the formula: 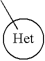
is a heterocyclic group represented by the formula: 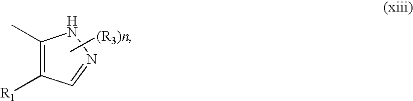
then R₁ should not be a halogen atom, and
10) when the group represented by the formula: 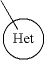
is a heterocyclic group represented by the formula: 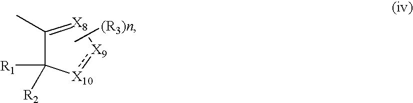
then at least one of R₁ and R₂ should be an optionally substituted aryl group or an optionally substituted heteroaryl group,
or a salt thereof.

2. A compound of the formula (i): 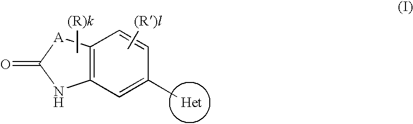
wherein
A is a group represented by the formula:
—X₁ [symbol] X₂ [symbol] X₃—
wherein
X₁ and X₂ are the same or different and each is a chemical bond, CH₂, CH, O, NH, N, S, SO or SO₂;
X₃ is CH₂, CH, O, NH, N, S, SO or SO₂; and [symbol]
is a single bond or a double bond;
provided that
when
—X₁ [symbol] X₂— is —X₁═X₂—,
then
—X₂ [symbol] X₃— should be —X₂—X₃—;
R and R′ are the same or different and each is an optionally substituted aliphatic hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group or an acyl group, or two R optionally form a spiro ring together with a carbon atom they are bonded to;
k is an integer of 0 to 4;
l is an integer of 0 to 3; and
a group represented by the formula: 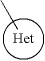
is a heterocyclic group represented by the formula: 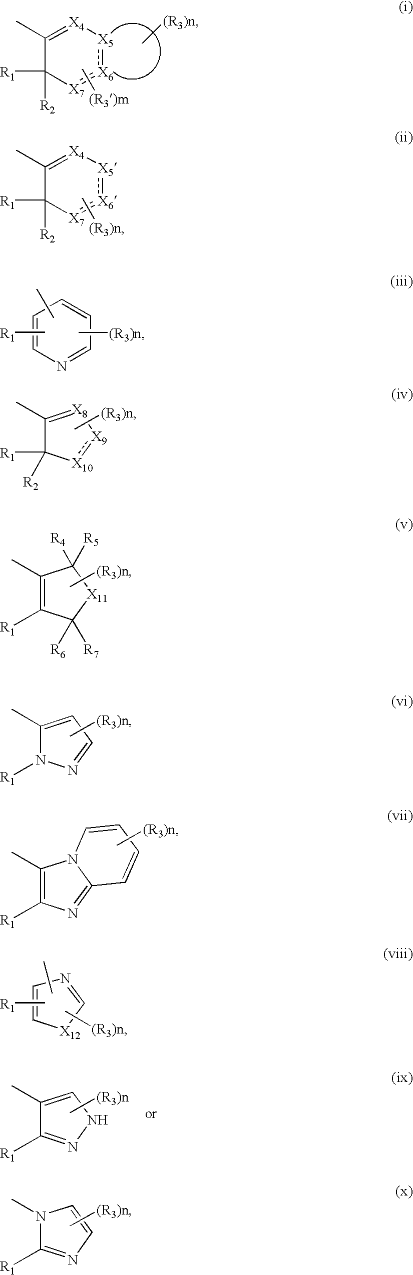
wherein
the formula: 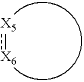
which partially constitutes the fused ring in the heterocyclic group represented by the formula (i), is a 5- to 7-membered ring which optionally contains, as a ring-constituting member, one or more members selected from O, N, S, SO and SO₂;
R₁ and R₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group, or
two R₃ optionally form, together with two adjacent atoms they are bonded to, a 3- to 7-membered ring which optionally contains, as a ring-constituting member, one or more members selected from O, N, S, SO and SO₂;
R₄ and R₅ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₄ and R₅ in combination optionally form an oxo group;
R₆ and R₇ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group, or
R₆ and R₇ in combination optionally form an oxo group;
provided that at least one of a pair of R₄ and R₅ and a pair of R₆ and R₇ should form an oxo group;
m and n are the same or different and each is an integer of 0 to 4;
X₄ is CH or N;
X₅ and X₆ are the same or different and each is CH, C or N;
X₅′ and X₆′ are the same or different and each is CH₂, CH, NH, N, O, S, SO or SO₂;
X₇ is CH₂, CH, NH, N, O, S, SO or SO₂;
Xₑ is CH or N;
X₉ is CH₂, CH, NH, N, O, S, SO or SO₂;
X₁₀ is CH₂, CH, NH, N, O, S, SO or SO₂;
X₁₁ is NH, O, S, SO or SO₂;
X₁₂ is O or S; and [symbol]
is a single bond or a double bond;
provided that
when
X₅ [symbol] X₆ is X₅═X₆;
then
X₆ [symbol] X₇ should be X₆—X₇, and
when
X₅′ [symbol] X₆′ is X₅′═X₆′,
then
X₆′ [symbol] X₇ should be X₆′—X₇;
with the proviso that
1) when the group represented by the formula: 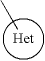
is a heterocyclic group represented by the formula: 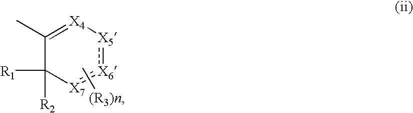
then at least one of R₁ and R₂ should be an optionally substituted aryl group or an optionally substituted heteroaryl group,
2) when the group represented by the formula: 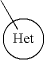
is a heterocyclic group represented by the formula: 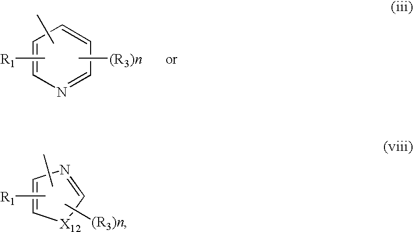
then the carbon atom to which the group represented by the formula: 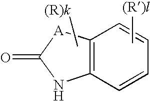
is bonded and the carbon atom to which R₁ is bonded should be adjacent to each other, and R₁ should be an optionally substituted aryl group or an optionally substituted heteroaryl group,
3) when the group represented by the formula:
—X₁ [symbol] X₂ [symbol] X₃— is —CH₂—O—, and the group represented by
the formula: 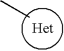
is a heterocyclic group represented by the formula: 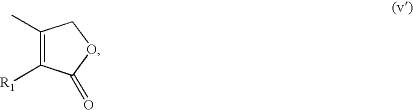
then R₁ should not be phenyl, 4-methoxyphenyl, 3,4-dimethoxyphenyl and 4-chlorophenyl,
4) when the group represented by the formula:
—X₁ [symbol] X₂ [symbol] X₃— is —CH₂—O—, and the group represented by the formula: 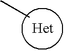
is a heterocyclic group represented by the formula: 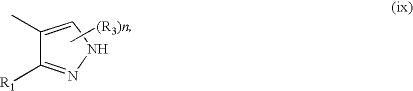
then R₁ should not be an optionally substituted 2-pyridyl,
5) when the group represented by the formula:
—X₁ [symbol] X₂ [symbol] X₃— is —CH₂—O—, and the group represented by the formula: 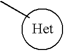
is a heterocyclic group represented by the formula: 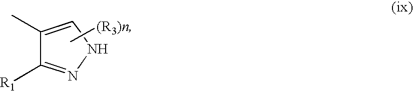
wherein R₁ is an optionally substituted phenyl, then —NH— group in the pyrazole ring as illustrated above should be substituted by R₃,
6) when the group represented by the formula:
—X₁ [symbol] X₂ [symbol] X₃— is —O—, —CH₂—O—, —CH₂—S— or —CH═CH—, and the group represented by the formula: 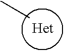
is a heterocyclic group represented by the formula: 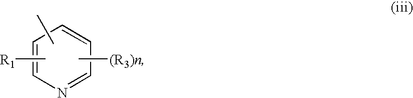
then R₁ should not be a halogen atom and trifluoromethyl,
7) when the group represented by the formula:
—X₁ [symbol] X₂ [symbol] X₃— is —NH— or —CH₂—NH—, and the group represented by the formula: 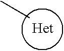
is a heterocyclic group represented by the formula: 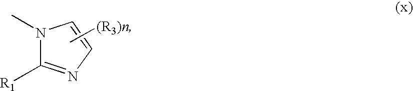
then R₁ should not be an alkyl group, and
8) when the group represented by the formula: 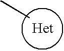
is a heterocyclic group represented by the formula: 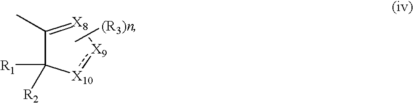
then at least one of R₁ and R₂ should be an optionally substituted aryl group or an optionally substituted heteroaryl group,
or a salt thereof.

3. The compound of claim 1, wherein none or one of X₁, X₂ and X₃ is hetero atom, or a salt thereof.

4. The compound of claim 1, wherein
A is a group represented by the formula:
—X₁ [symbol] X₂ [symbol] X₃—
wherein
X₁ is a chemical bond or CH₂;
X₂ is a chemical bond, CH₂, CH, O, NH, N, S, SO or SO₂; and
X₃ is CH₂, CH, O, NH, N, S, SO or SO₂;
or a salt thereof.

5. The compound of claim 1, excluding a compound wherein consecutive three or more of X₄, X₅, X₆ and X₇ or consecutive three or more of X₄, X₅′, X₆′ and X₇ are hetero atoms, or a salt thereof.

6. The compound of claim 1, wherein
the group represented by the formula: 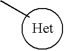
is a heterocyclic group represented by the formula: 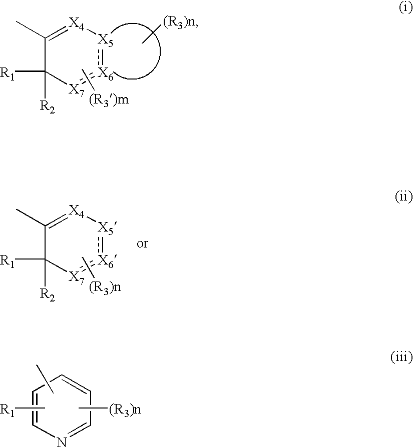
wherein R₁, R₂, R₃, R₃′, m, n, X₄, X₅, X₅′, X₆, X₆′ and
X₇ are each as defined in claim 1.
or a salt thereof.

7. The compound of claim 1, wherein
the group represented by the formula: 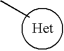
is a heterocyclic group represented by the formula: 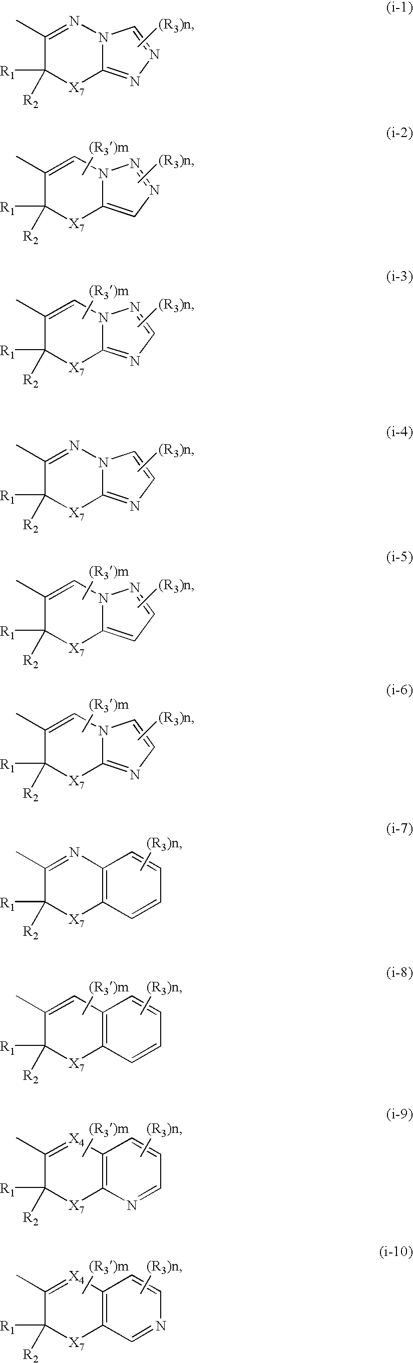 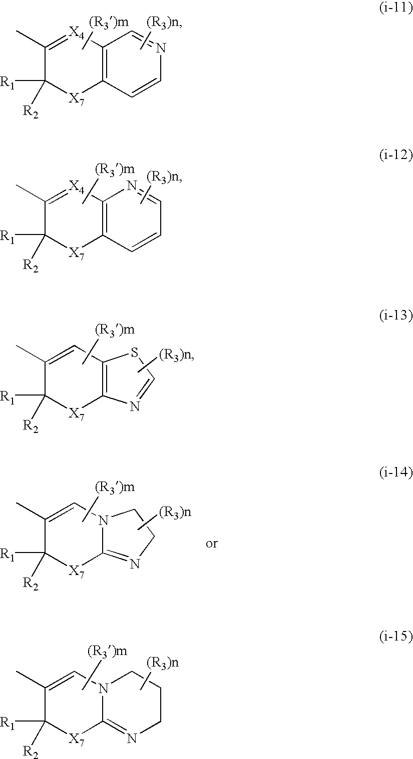
wherein
R₁, R₂, n and X₄ are each as defined in claim 1;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;
X₇ is O, S, SO or SO₂; and
m is an integer of 0 to 1,
or a salt thereof.

8. The compound of claim 1, wherein
the group represented by the formula: 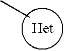
is a heterocyclic group represented by the formula: 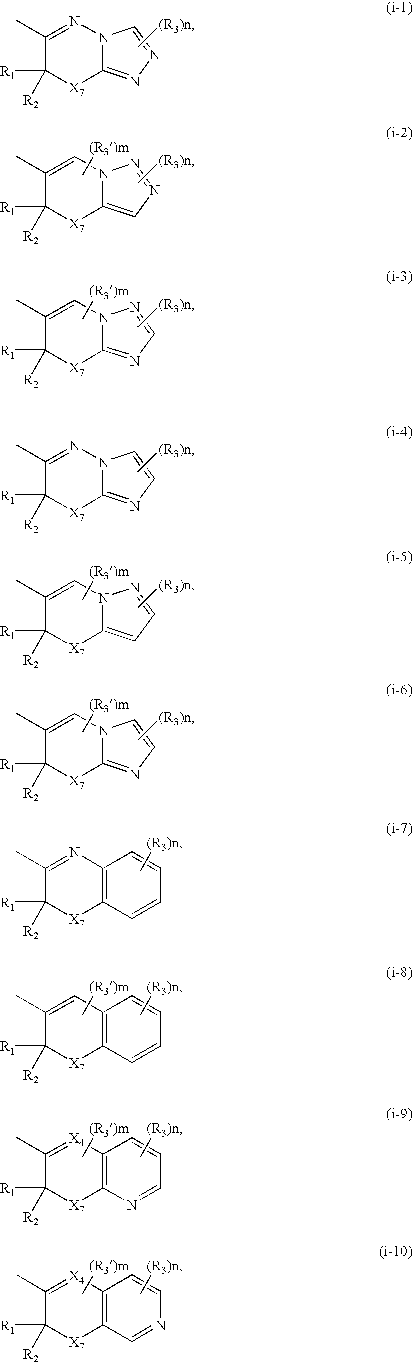 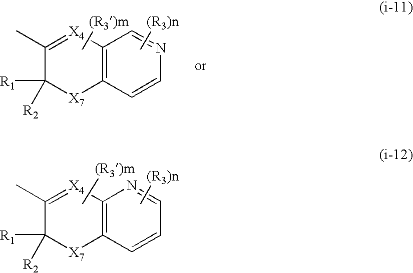
wherein
R₁, R₂, n and X₄ are each as defined in claim 1;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group;
X₇ is O, S, SO or SO₂; and
m is an integer of 0 to 1,
or a salt thereof.

9. The compound of claim 1, wherein
the group represented by the formula: 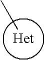
is a heterocyclic group represented by the formula: 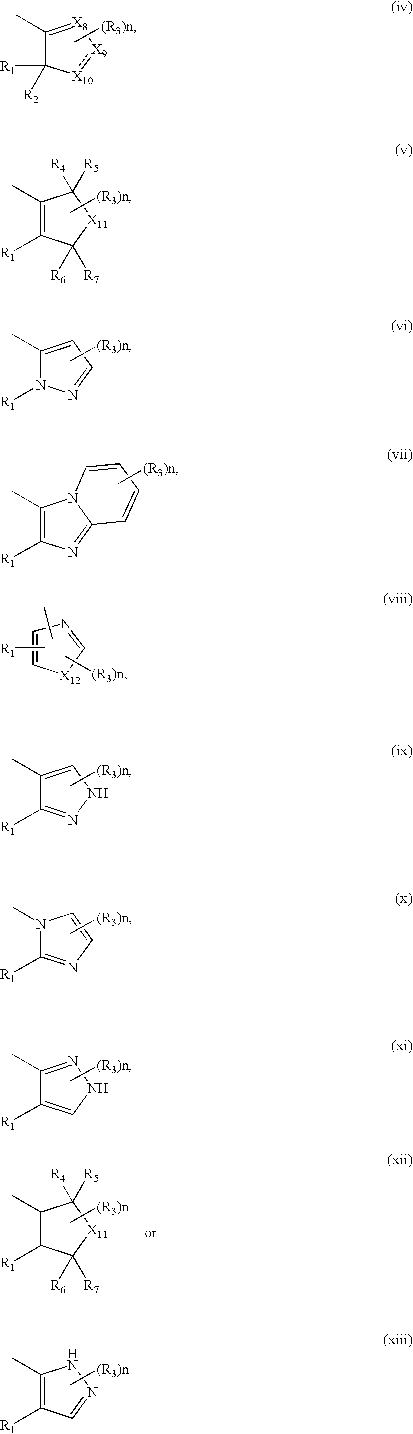
wherein R₁, R₂, R₃, R₄, R₅, R₆, R₇, n, X₈, X₉, X₁₀, X₁₁ and X₁₂ are each as defined in claim 1.
or a salt thereof.

10. The compound of claim 1, wherein
the group represented by the formula: 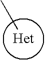
is a heterocyclic group represented by the formula: 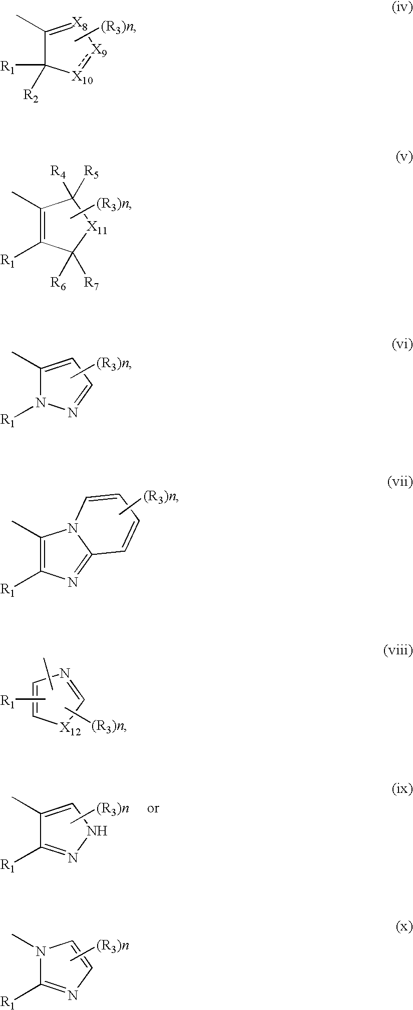
wherein R₁, R₂, R₃, R₄, R₅, R₆, R₇, n, X₈, X₉, X₁₀, X₁₁ and X₁₂ are each as defined in claim 1.
or a salt thereof.

11. The compound of claim 1, wherein
the group represented by the formula: 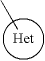
is a heterocyclic group represented by the formula: 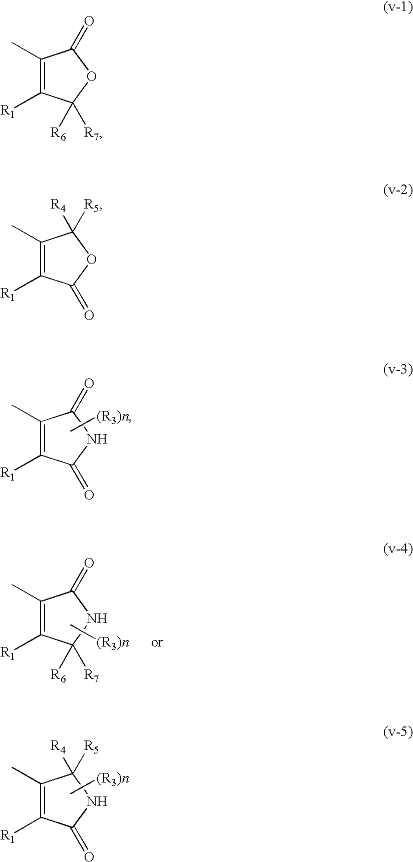
wherein
R₁, R₄, R₅, R₆ and R₇ are as defined in claim 1;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted mercapto group, an acyl group or an optionally substituted cyclic group; and
n is an integer of 0 to 1,
or a salt thereof.

12. The compound of claim 1, wherein
the group represented by the formula: 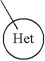
is a heterocyclic group represented by the formula: 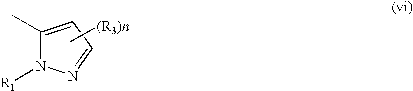
wherein
R₁ is as defined in claim 1;
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group; and
n is an integer of 0 to 2,
or a salt thereof.

13. The compound of claim 1, wherein
the group represented by the formula: 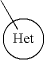
is a heterocyclic group represented by the formula: 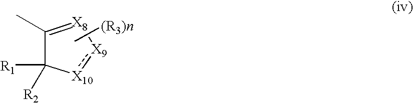
wherein
R₁, R₂, n, X₈, X₉ and X₁₀ are each as defined in claim 1; and
R₃ is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group,
or a salt thereof.

14. The compound of claim 1, wherein
the group represented by the formula: 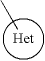
is a heterocyclic group represented by the formula: 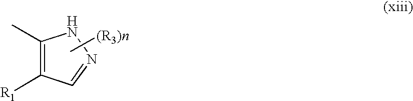
wherein
R₁ is as defined in claim 1;
R₃ is an Optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group; and
n is an integer of 0 to 2,
or a salt thereof.

15. The compound of claim 1, wherein
the group represented by the formula: 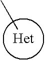
is a heterocyclic group represented by the formula: 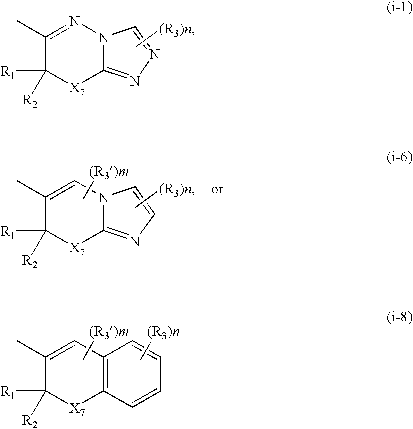
wherein
R₁, R₂ and n are each as defined in claim 1;
R₃ and R₃′ are the same or different and each is an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an oxo group, an optionally substituted imino group, an optionally substituted mercapto group, an acyl group or an optionally substituted cyclic group;
X₇ is O, S, SO or SO₂; and
m is an integer of 0 to 1,
or a salt thereof.

16. The compound of claim 1, wherein
when one of R₁ and R₂ is a hydrogen atom, then the other should not be a hydrogen atom,
or a salt thereof.

17. 6-(7-phenyl-7H-[1,2,4]triazolo[3,4-b][1,3,4]thiadiazin-6-yl)-2H-1,4-benzoxazin-3(4H)-one,
6-[2-(4-fluorophenyl)-2H-thiochromen-3-yl]-2H-1,4-benzoxazin-3(4H)-one,
3-(3-oxo-3,4-dihydro-2H-1,4-benzoxazin-6-yl)-2-phenyl-2H-thiochromene-7-carbonitrile,
6-(2-amino-6-phenyl-6H-1,3-thiazin-5-yl)-2H-1,4-benzoxazin-3(4H)-one,
6-[7-(2-chlorophenyl)-7H-[1,2,4]triazolo[3,4-b][1,3,4]thiadiazin-6-yl]-2H-1,4-benzoxazin-3(4H)-one,
8-fluoro-6-[7-(4-fluorophenyl)-7H-[1,2,4]triazolo[3,4-b][1,3,4]thiadiazin-6-yl]-2H-1,4-benzoxazin-3(4H)-one,
6-[7-(4-fluorophenyl)-7H-[1,2,4]triazolo[3,4-b][1,3,4]thiadiazin-6-yl]-8-methyl-2H-1,4-benzoxazin-3(4H)-one,
8-chloro-6-[7-(4-fluorophenyl)-7H-imidazo[2,1-b][1,3]thiazin-6-yl]-2H-1,4-benzoxazin-3(4H)-one,
3-(4-fluorophenyl)-4-(3-oxo-3,4-dihydro-2H-benzo[b][1,4]oxazin-6-yl)-1-phenyl-1H-pyrrole-2,5-dione,
6-(1-o-tolyl-3-(trifluoromethyl)-1H-pyrazol-5-yl)-2H-benzo[b][1,4]oxazin-3(4H)-one,
6-(1-(4-fluoro-2-methylphenyl)-3-(trifluoromethyl)-1H-pyrazol-5-yl)-2H-benzo[b][1,4]oxazin-3(4H)-one,
8-fluoro-6-(1-(4-fluoro-2-methylphenyl)-3-(trifluoromethyl)-1H-pyrazol-5-yl)-2H-benzo[b][1,4]oxazin-3(4H)-one,
6-(1-(4-fluoro-2-methylphenyl)-3-(trifluoromethyl)-1H-pyrazol-5-yl)-8-methyl-2H-benzo[b][1,4]oxazin-3(4H)-one,
6-(1,3-dimethyl-4-phenyl-1H-pyrazol-5-yl)-2H-benzo[b][1,4]oxazin-3(4H)-one,
6-(1-(4-chloro-2-methylphenyl)-3-(trifluoromethyl)-1H-pyrazol-5-yl)-2H-benzo[b][1,4]oxazin-3(4H)-one,
6-(1-(2,5-dimethylphenyl)-3-(trifluoromethyl)-1H-pyrazol-5-yl)-2H-benzo[b][1,4]oxazin-3(4H)-one,
6-[1-(4-fluoro-2-methylphenyl)-3-(trifluoromethyl)-1H-pyrazol-5-yl]-8-methyl-2H-pyrido[3,2-b][1,4]oxazin-3(4H)-one, or
6-(3-(1,1-difluoroethyl)-1-(4-fluoro-2-methylphenyl)-1H-pyrazol-5-yl)-8-methyl-2H-benzo[b][1,4]oxazin-3(4H)-one,
or a salt thereof.

18. A prodrug of a compound of claim 1 or a salt thereof.

19. A pharmaceutical composition comprising a compound of claim 1 or a pharmaceutically acceptable salt thereof or a prodrug thereof, in admixture with a pharmaceutically acceptable carrier.

20. A method for inhibiting the mineralocorticoid receptor activity in a mammal, comprising administering an effective amount of a compound of claim 1 or a pharmaceutically acceptable salt thereof or a prodrug thereof to said mammal.

21. A method for preventing or treating a disease or condition mediated by the mineralocorticoid receptor activation in a mammal, comprising administering an effective amount of a compound of claim 1 or a pharmaceutically acceptable salt thereof or a prodrug thereof to said mammal.

22. A method for inhibiting the mineralocorticoid receptor activity in a mammal, comprising administering an effective amount of a compound of formula (Ia′): 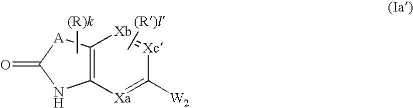
wherein
X_{c}′ is C—W₁ or N;
W₁ and W₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
l′ is an integer of 0 to 2; and
A, R, R′ Xₐ, X_{b} and k are each as defined in claim 1;
with the proviso that
1) at least one of W₁ and W₂ should be an optionally substituted cyclic group,
2) when W₂ is a hydrogen atom, then W₁ should not be an optionally substituted phenyl, and
3) at least one of Xₐ, X_{b} and X_{c}′ should be N,
a pharmaceutically acceptable salt thereof or a prodrug thereof to said mammal.

23. A method for inhibiting the mineralocorticoid receptor activity in a mammal, comprising administering an effective amount of a compound of formula (I′): 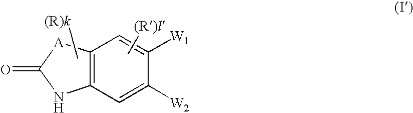
wherein
W₁ and W₂ are the same or different and each is a hydrogen atom, an optionally substituted aliphatic chain hydrocarbon group, an optionally substituted hydroxy group, an optionally substituted amino group, an optionally esterified carboxyl group, an optionally substituted carbamoyl group, a halogen atom, a nitro group, a cyano group, an optionally substituted mercapto group, an acyl group or an optionally substituted cyclic group;
l′ is an integer of 0 to 2; and
A, R, R′ and k are each as defined in claim 2;
with the proviso that
1) at least one of W₁ and W₂ should be an optionally substituted cyclic group, and
2) when W₂ is a hydrogen atom, then W₁ should not be an optionally substituted phenyl,
a pharmaceutically acceptable salt thereof or a prodrug thereof to said mammal.
